(19) United States
(12) Patent Application Publication
(10) Pub. No.: US 2007/0077641 A1
(43) Pub. Date: Apr. 5, 2007

(54) Methionine aminopeptidase and methods of use

(76) Inventor: Leslie Palmer, (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/555,830

(22) PCT Filed: May 7, 2004
(86) PCT No.: PCT/US04/14258

§ 371 Date: Nov. 7, 2005

Related U.S. Application Data
(60) Provisional application No. 60/468,643, filed on May 7, 2003.

(51) Int. Cl. C12N 9/22; G06F 19/00

(52) U.S. Cl. 435/199; 702/19

(57) ABSTRACT

Invented are non-peptide TPO mimetics. Also invented is a method of treating thrombocytopenia, in a mammal, including a human, in need thereof which comprises administering to such mammal an effective amount of a selected hydroxy-1-azobenzene derivative.

### FIELD OF THE INVENTION

The present invention relates to the identification of novel bacterial methionine aminopeptidase (herein “MetAP”) crystalline structures. In particular, it provides novel methionine aminopeptidase active sites of crystalline structures and active sites of crystalline structures in complex with inhibitors and methods to use these crystalline forms and their active sites to identify and improve methionine aminopeptidase inhibitor compounds, among other uses. These compounds are characterized by the ability to competitively inhibit binding of substrates or other like-molecules to the active site of MetAP, a member of the aminopeptidase family.

### BACKGROUND OF THE INVENTION

Methionine aminopeptidases are ubiquitously distributed in all living organisms. They catalyze the removal of the initiator methionine from newly translated polypeptides using divalent metal ions as cofactors. Two distantly related MetAP enzymes, type 1 and type 2, are found in eukaryotes, which at least in yeast, are both required for normal growth; whereas one MetAP is currently known in eubacteria (type 1) and archaebacteria (type 2). The N-terminal extension region distinguishes the MetAPs in eukaryotes from those in procaryotes. A 64-amino acid sequence insertion (from residues 381 to 444 in hMetAP2) in a catalytic C-terminal domain distinguishes the MetAP-2 family from the MetAP-1 family. Despite the difference in the gene structure, MetAP enzymes appear to share a highly conserved catalytic scaffold termed “pita-bread” fold (Bazan, et al. (1994) Proc. Natl. Acad. Sci. U.S.A. 91, 2473) containing six strictly conserved residues implicated in the coordination of the metal cofactors.

N-terminal methionine removal in bacteria is a two-step process requiring first the removal on the N-formyl group by polypeptide deformylase followed by cleavage of the N-terminal methionine when the adjacent amino acid is small (e.g., Ala, Pro, Ser, Thr, Gly, Cys, and Val). Both of these steps appear to be essential for cell viability. Failure to remove the N-terminal methionine can lead to inactive enzymes (e.g., glutamine phosphoribosylpyrophosphate amidotransferase and N-terminal nucleophile hydrolases). Therefore, inhibition or other modulation of MetAP may have a wide-ranging effect inhibiting or otherwise modulating the action of essential enzymes involved in varied cellular processes.

MetAP is an attractive antibacterial target as this enzyme has been demonstrated to be essential for bacterial growth in vitro (Chang, et al. (1989) J. Bacteriol. 171, 4071, and Miller et al. (1989) J. Bacteriol. 171, 5215.); and appears to be universally conserved in prokaryotes. This indicates that inhibitors or other modulators directed against this target will be broad-spectrum agents and will kill bacteria. Further, this inventions provides that this gene may be transcribed in thigh lesion and pyelonephritis models of infection with S. aureus as well as both early and late in murine respiratory tract infection with S. pneumoniae. These models indicate the importance of this process in infection.

### SUMMARY OF THE INVENTION

In one aspect, the present invention relates to methionine aminopeptidase (herein “MetAP”) crystalline structures, for example, a MetAP in crystalline form derived from S. aureus or S. pneumoniae.

In another aspect, the present invention provides a crystalline form of a S. aureus methionine aminopeptidase in complex with a MetAP inhibitor or other modulator, for example a triazole, for example, a 1,2,3 triazole.

In another aspect, the present invention provides a crystalline form of a S. aureus methionine aminopeptidase in complex with an inhibitor or other modulator, 5-benzofuran-2-yl-1-H-[1,2,3]triazole or 5-(3-Iodo-phenyl)-1-H-[1,2,3]triazole.

In yet another aspect, the invention provides a role for residues in an active site responsible for binding of aminopeptidase inhibitors or other modulators by MetAP.

In yet another aspect, the invention provides a method of modulating an activity of a bacterial MetAP comprising administering to a mammal in need thereof a compound that spatially fits into an active site of MetAP.

In yet another aspect, the invention provides a structural basis to identify positions of amino acid residues and metals bound to those residues with respect to an inhibitor or other modulator and a method for identifying inhibitors or other modulators of MetAP.

Another aspect of this invention comprises machine-readable media encoded with data representing coordinates of a three-dimensional structure of a MetAP crystal structure alone or in complex with an inhibitor or other modulator.

A further aspect of the invention provides for a Staphylococcus aureus MetAP defined by three dimensional protein coordinates of Table I in an essentially pure form or a homolog thereof.

Another aspect of this invention includes a Staphylococcus aureus MetAP wherein the MetAP crystal form comprises cubic crystals with a space group 123.

In yet another aspect, the invention provides a Staphylococcus aureus MetAP wherein cubic crystals comprise a lattice constant of about a=121.36 Ångstroms (Å).

Another aspect of this invention includes a Staphylococcus aureus MetAP wherein a MetAP crystal form comprises monoclinic crystals with a space group P2₁ and lattice constants of about a=41.19 Å, about b=76.78 Å, and about c=41.71 Å, β=104.165°.

Another aspect of this invention includes a Staphylococcus aureus MetAP wherein a MetAP crystal form is in complex with a MetAP inhibitor or other modulator, for example, 5-(3-Iodo-phenyl)-1-H-[1,2,3]triazole and 5-benzofuran-2-yl-1-H-[1,2,3]triazole.

A further aspect of the invention provides a Streptococcus pneumoniae MetAP defined by three dimensional protein coordinates of Table VIII in an essentially pure form, partially pure form, pure form, or a homolog of any thereof.

Another aspect of this invention includes a Streptococcus pneumoniae MetAP wherein a crystal form comprises orthorhombic crystals with a space group P2₁2₁2₁, wherein said orthorhombic crystals comprise lattice constants of about a=56.77 Å, about b=69.16 Å, and about c=80.51 Å.

In yet another aspect, the invention provides a Streptococcus pneumoniae MetAP in complex with a MetAP inhibitor or other modulator, such as a triazole, for example a 1,2,3 triazole.

Another aspect of this invention includes a process for determining a bacterial MetAP crystalline form of other bacteria or species by using structural coordinates of a Staphylococcus aureus MetAP crystal or portions thereof, to determine a crystal structure of a mutant, homologue, or co-complex of a binding pocket or active site by molecular replacement.

Another aspect of this invention includes a process for determining a bacterial MetAP crystalline form of other bacteria or species by using structural coordinates of a Streptococcus pneumoniae MetAP crystal or portions thereof, to determine a crystal structure of a mutant, homologue, or co-complex of a binding pocket or active site by molecular replacement.

A further aspect of the invention provides a process of identifying a bacterial MetAP inhibitor or other modulator capable of binding to and inhibiting or otherwise modulating an enzymatic activity of a bacterial MetAP said process comprising:
introducing into a suitable computer program information defining an active site conformation of a MetAP molecule comprising a conformation defined by crystal coordinates comprising those listed in Tables I to X wherein said program displays a three-dimensional structure;
creating a three dimensional structure of a test compound in said computer program;
displaying and superimposing a model of said test compound on a model of said active site;
incorporating said test compound in a biological methionine aminopeptidase activity assay for a methionine aminopeptidase characterized by said active site; and
determining whether said test compound inhibits or otherwise modulates an enzymatic activity in said assay.

Another aspect of this invention includes a process for designing inhibitors or other modulators of MetAP activity using atomic coordinates of a bacterial MetAP in crystalline form to computationally evaluate a chemical entity for associating with an active site of a MetAP enzyme.

In yet another aspect, the invention provides a method of modifying a test bacterial MetAP polypeptide comprising:
providing a test bacterial MetAP polypeptide sequence having a characteristic that is targeted for modification;
   aligning the test bacterial MetAP polypeptide sequence with a reference bacterial MetAP polypeptide sequence for which an X-ray structure is available, wherein the reference bacterial MetAP polypeptide sequence has a characteristic that is desired for the test bacterial MetAP polypeptide;
   building a three-dimensional model for the test bacterial MetAP polypeptide using the three-dimensional coordinates of the X-ray structure(s) of a reference bacterial MetAP polypeptide and its sequence alignment with the test bacterial MetAP polypeptide sequence;
   examining the three-dimensional model of the test bacterial MetAP polypeptide for a difference in an amino acid residue as compared to a reference bacterial MetAP polypeptide, wherein the residues are associated with the desired characteristic; and
   mutating an amino acid residue in the test bacterial MetAP polypeptide sequence located at a difference identified in step (d) to a residue associated with the desired characteristic, whereby the test bacterial MetAP polypeptide is modified.

Another aspect of the invention provides a method for the treatment of an individual having need to inhibit or other wise modulate a bacterial methionine aminopeptidase comprising: administering to the individual an amount, for example, a therapeutically effective amount, of a compound that binds to, alters the structure of, or interacts with, an active site of a bacterial MetAP enzyme. In a further embodiment, a compound is selected from the group consisting of 5-(3-Iodo-phenyl)-1-H-[1,2,3]triazole and 5-benzofuran-2-yl-1-H-[1,2,3]triazole, or a pharmaceutically active salt or solvate thereof.

In yet another aspect, the invention provides a method of drug design comprising using the structural coordinates of a MetAP crystal to computationally evaluate a chemical entity for associating with the inhibitor or modulator binding site of MetAP.

A further aspect of the invention provides a method for modulating an activity of a bacterial methionine aminopeptidase comprising: contacting a methionine aminopeptidase with a compound that binds to, alters a structure of, or interacts with, an active site of a bacterial MetAP enzyme and modulates said activity of said methionine aminopeptidase. In a further embodiment, a compound is selected from the group consisting of 5-(3-Iodo-phenyl)-1-H-[1,2,3]triazole and 5-benzofuran-2-yl-1-H-[1,2,3]triazole, or a pharmaceutically active salt or solvate thereof. Another aspect of this invention provides a method for modulating an activity of a bacterial methionine aminopeptidase comprising: contacting a methionine aminopeptidase with a compound that comprises an activity selected from the group consisting of: bonding with, complexing with, coordinating with and/or cocrystalizing with an amino acid residue or residues defined by a protein coordinate or coordinates set forth in Table I; bonding with, complexing with, coordinating with and/or cocrystalizing with a Staphylococcus aureus MetAP crystal form comprising cubic crystals comprising a space group I23; bonding with, complexing with, coordinating with and/or cocrystalizing with a Staphylococcus aureus MetAP cubic crystal comprising a lattice constant of about a=121.36 Ångstroms (Å); bonding with, complexing with, coordinating with and/or cocrystalizing with a Staphylococcus aureus MetAP crystal form comprising a monoclinic crystal with a space group P2₁ and lattice constants of about a=41.19 Å, about b=76.78 Å, and about c=41.71 Å, β=104.165°; bonding with, complexing with, coordinating with and/or cocrystalizing with an amino acid residue or residues defined by a protein coordinate or coordinates set forth in Table VII; bonding with, complexing with, coordinating with and/or cocrystalizing with a Streptococcus pneumoniae MetAP crystal form comprising orthorhombic crystals with a space group P2₁2₁2₁, wherein said orthorhombic crystals comprise lattice constants of about a=56.77 Å, about b=69.16 Å, and about c=80.51 Å; bonding with, coordinating with and/or cocrystalizing with a Streptococcus pneumoniae MetAP in complex with a MetAP inhibitor or other modulator; and bonding with, complexing with, coordinating with and/or cocrystalizing with a bacterial MetAP enzyme.

A further embodiment of the inventions provides a compound or composition comprising a compound that modulates an activity of a bacterial methionine aminopeptidase, wherein said activity comprises binding to, altering a structure of, or interacting with, an active site of a bacterial MetAP enzyme, for example, a compound selected from the group consisting of 5-(3-Iodo-phenyl)-1-H-[1,2,3]triazole and 5-benzofuran-2-yl-1-H-[1,2,3]triazole, or a pharmaceutically active salt or solvate thereof.

Another aspect of this invention provides a compound or composition comprising a compound that modulates an activity of a bacterial methionine aminopeptidase, wherein said activity is selected from the group consisting of: bonding with, complexing with, coordinating with and/or cocrystalizing with an amino acid residue or residues defined by a protein coordinate or coordinates set forth in Table I; bonding with, complexing with, coordinating with and/or cocrystalizing with a Staphylococcus aureus MetAP crystal form comprising cubic crystals comprising a space group I23; bonding with, complexing with, coordinating with and/or cocrystalizing with a Staphylococcus aureus MetAP cubic crystal comprising a lattice constant of about a=121.36 Ångstroms (Å); bonding with, complexing with, coordinating with and/or cocrystalizing with a Staphylococcus aureus MetAP crystal form comprising a monoclinic crystal with a space group P2₁ and lattice constants of about a=41.19 Å, about b=76.78 Å, and about c=41.71 Å, β=104.165°; bonding with, complexing with, coordinating with and/or cocrystalizing with an amino acid residue or residues defined by a protein coordinate or coordinates set forth in Table VIII; bonding with, complexing with, coordinating with and/or cocrystalizing with a Streptococcus pneumoniae MetAP crystal form comprising orthorhombic crystals with a space group P2₁2₁2₁, wherein said orthorhombic crystals comprise lattice constants of about a=56.77 Å, about b=69.16 Å, and about c=80.51 Å; bonding with, coordinating with and/or cocrystalizing with a Streptococcus pneumoniae MetAP in complex with a MetAP inhibitor or other modulator; and bonding with, complexing with, coordinating with and/or cocrystalizing with a bacterial MetAP enzyme.

### DESCRIPTION OF THE FIGURES

FIG. 1 is a ribbon diagram of S. aureus methionine aminopeptidase. Amino and carboxyl-termini are indicated by N and C. The drawing was produced using the program MOLSCRIPT [Kraulis, P., J. Appl. Crystallogr., 24, 946-950 (1991)].

FIG. 2 is an illustration of an active site of S. aureus methionine aminopeptidase.

FIG. 3 is a stereoview of an active site of S. aureus methionine aminopeptidase. For clarity, no hydrogen atoms or water molecules are shown.

FIG. 4 is an illustration of an active site of S. aureus methionine aminopeptidase in complex with an inhibitor, 5-(3-Iodo-phenyl)-1-H-[1,2,3]triazole, of S. aureus methionine aminopeptidase. The exemplary view depicts an interaction of this inhibitor with atoms of residues of an active site of S. aureus methionine aminopeptidase within 5 Å of this inhibitor. For clarity, no hydrogen atoms or water molecules are shown. 2 active site metal atoms are represented as spheres.

FIG. 5 is a stereoview of an active site of S. aureus methionine aminopeptidase in complex with an inhibitor, 5-(3-Iodo-phenyl)-1-H-[1,2,3]triazole, of S. aureus methionine aminopeptidase. This figure is a stereo drawing of an interaction of this inhibitor with atoms of residues of an active site of S. aureus methionine aminopeptidase within 5 Å of this inhibitor. Water molecules and 2 active site metal atoms are shown as crosses in the figure.

FIG. 6 is an illustration of an active site of S. aureus methionine aminopeptidase in complex with an inhibitor, 5-benzofuran-2-yl-1-H-[1,2,3]triazole, of S. aureus methionine aminopeptidase. This view depicts an interaction of an inhibitor with atoms of residues of an active site of S. aureus methionine aminopeptidase within 5 Å of this inhibitor. For clarity, no hydrogen atoms or water molecules are shown. 2 active site metal atoms are represented as spheres.

FIG. 7 is a stereoview of an active site of S. aureus methionine aminopeptidase in complex with an inhibitor, 5-benzofuran-2-yl-1-H-[1,2,3]triazole, of S. aureus methionine aminopeptidase. This figure is a stereo drawing of an interaction of this inhibitor with atoms of residues of an active site of S. aureus methionine aminopeptidase within 5 Å of this inhibitor. Water molecules and 2 active site metal atoms are shown as crosses in the figure.

FIG. 8 is a ribbon diagram of S. pneumoniae methionine aminopeptidase. Amino and carboxyl-termini are indicated by N and C respectively.

FIG. 9 is an illustration of an active site of S. pneumoniae methionine aminopeptidase.

FIG. 10 is a stereoview of an active site of S. pneumoniae methionine aminopeptidase. For clarity, no hydrogen atoms or water molecules other than a water molecule bridging metal atoms are shown.

### DETAILED DESCRIPTION OF THE INVENTION

The present invention provides a method for inhibiting or otherwise modulating bacterial methionine aminopeptidase (MetAP) by administering compounds with certain structural, physical and/or spatial characteristics that allow for an interaction of said compounds with specific residues of an inhibitor or modulator binding site of a bacterial methionine aminopeptidase. This interaction inhibits or otherwise modulates an activity of bacterial MetAP and, thus, treats diseases where bacterial replication is a factor.

The present invention provides for bacterial MetAP crystalline structures, and methods for identifying inhibitors or modulators of MetAP that bind to or interacts with an active site of a bacterial MetAP enzyme. In addition, the invention provides for active sites of a crystalline structure of MetAP, in complex with inhibitor or other modulator compounds and methods to use these crystalline forms and their active sites to identify and improve MEtAP inhibitor or other modulator compounds, such as peptide, peptidomimetic or synthetic compositions. These compounds may be characterized by an ability to competitively inhibit binding of substrates or other like-molecules to an active site of MetAP.

Crystallization and Structure Solution of S. pneumoniae Methionine Aminopeptidase

Examplary crystals of S. pneumoniae methionine aminopeptidase grew to a size of approximately 0.2 mm³ overnight. In this example, the concentration of S. pneumoniae methionine aminopeptidase used in crystallization was approximately 15 mg/ml. A method of vapor diffusion in sitting drops was used to grow crystals from ta solution of S. pneumoniae methionine aminopeptidase. Crystals grew at root temperature from drops containing protein in a solution of 10% glycerol in 20 mM Hepes buffer at pH 7.4 containing 0.10M NaCl, 0.5 mM CoCl₂. This solution was mixed in equal volumes with a reservoir solution of 10% isopropanol, 20% PEG 4000 and 100 mM Hepes at pH 7.5. Crystals are orthorhombic, space group P2₁2₁2₁, with cell constants of a=56.77, b=69.16, c=40.51 Ångstroms. These exemplary crystals contained one molecule in the asymmetric unit and approximately 57% solvent with a Vₘ value of 2.93 A³/Dalton. X-ray diffraction data were measured from a single crystal using synchrotron radiation provided by beamline 17-ID at the Advanced Photon Source, Argonne National Laboratory. A structure was determined by molecular replacement using CNX (Molecular Simulations Inc). A starting model consisted of all protein atoms of the structure of S. aureus methionine aminopeptidase determined as described below. This exemplary model was refined by rigid-body refinement, and resulting phases were used to calculate Fourier maps with coefficients IFₒ-F_{c}I and I2Fₒ-F_{c}I, into which an atomic model of S. pneumoniae methionine aminopeptidase was built using a molecular graphics system XtalView (Molecular Simulations Inc). Conventional positional refinement was carried out during protein model building using CNX to a final R_{c}-value of 0.22 at 1.0 Ångstroms resolution.

Crystallization and Structure Solution of S. aureus Methionine Aminopeptidase

Exemplary crystals of S. aureus methionine aminopeptidase grew under two different conditions. For example, Form I crystals grew to a size of approximately 0.2 mm³ in about two days. The concentration of S. aureus methionine aminopeptidase used in this exemplary crystallization was approximately 12 mg./ml. A method of vapor diffusion in sitting drops was used to grow crystals from a solution of S. aureus methionine aminopeptidase. Crystals grew at room temperature from drops containing protein in a solution of 10% glycerol in 10 mM Hepes buffer at pH 7.4 containing 0.20M NaCl, 1 mM CoCl₂. This solution was mixed in equal volumes with a reservoir solution of 30% peg 400, 0.2M NaCl and 0.1 M Hepes at pH 7.5. Exemplary crystals of Form I comprise a cubic, space group 123, with cell constants of a=121.36 Ångstroms. Exemplary crystals contain one molecule in the asymmetric unit and approximately 53% solvent with a Vₘ value of 2.71 A³/Dalton. X-ray diffraction data were measured from a single crystal using synchrotron radiation provided by beamline 17-ID at the Advance Photon Source, Argonne National Laboratory. A structure was determined by molecular replacement using CNX (Molecular Simulations Inc). A starting model consisted of all protein atoms of the published structure of E. coli methionine aminopeptidase published by Roderick and Matthews [S. L. Roderick, B. W. Matthews. Structure of the Cobalt-dependent Methionine Aminopeptidase from Escherichia coli: a New Type of Proteolytic Enzyme, Biochemistry 32, 3907 (1993)]. This model was refined by rigid-body refinement, and resulting phases were used to calculate Fourier maps with coefficients IFₒ-F_{c}I and I2Fₒ-F_{c}I, into which an atomic model of S. aureus methionine aminopeptidase was built using a molecular graphics system XtalView (Molecular Simulations Inc). Conventional positional refinement was carried out during protein model building using CNX to a final R_{c}-value of 0.25 at 2.5 Ångstroms resolution.

Exemplary Form II crystals grew to a size of approximately 0.2 mm³ in about two days at room temperature. The concentration of S. aureus methionine aminopeptidase used in crystallization was approximately 12 mg/ml. A method of vapor diffusion in sitting drops was used to grow crystals from the solution of S. aureus methionine aminopeptidase. Exemplary crystals grew from drops containing protein in a solution of 10% glycerol in 10 mM Hepes buffer at pH 7.4 containing 0.20M NaCl, 1 mM CoCl₂. This solution was mixed in equal volumes with absolution of 18% PEG 8000, 0.12M Na-acetate, 0.06M Na-cacodylate-acetate, pH 6.5 and equilibrate with a reservoir containing 15% PEG 8000, 0.10M Na-acetate, 0.05M Na-cacodylate-acetate, pH 6.5. These crystals of Form II comprise a monoclinic space group P2₁, with cell constants of a=41.19, b=76.78, and c=41.71 Ångstroms β=104.165°. Crystals contain a molecule in an asymmetric unit and approximately 46% solvent with a Vₘ value of 2.33 A³/Dalton. X-ray diffraction data were measured from a single crystal using synchrotron radiation provided by beamline 17-ID at the Advance Photon Source, Argonne National Laboratory. A structure was determined by molecular replacement as described above. Conventional positional refinement was carried out using CNX to a final R_{c}-value of 0.22 at 1.8 Ångstroms resolution.

Structure Solution of S. aureus Methionine Aminopeptidase Inhibitor Complexes

Exemplary complexes were prepared by introducing solid inhibitor into a crystal mother liquor after crystal formation and allowed to incubate for 24 to 48 hours. Form II crystal were used to determine the structure of inhibitor complexes of S. aureus methionine aminopeptidase. Structures were refined as described above at 1.8 Ångstroms resolution.

Abbreviations

mM, milliMolar
R_{c}=Σ|(Fₒ−F_{c})|/Fₒ
Fₒ=observed structure amplitude
F_{c}=calculated structure amplitude

The present invention also provides bacterial MetAP crystalline structures in complex with inhibitors and provides methods to use these crystalline forms to identify and improve bacterial MetAP inhibitor compounds. Such compounds are characterized by their ability to inhibit MetAP activity.

It has now been discovered that substituted triazoles, for example, substituted 1,2,3-triazoles of formula (I) and formula (IA) are inhibitors of bacterial MetAP. It has also now been discovered that selective inhibition of MetAP enzyme mechanisms by treatment with inhibitors of formula (I) and formula (IA), or a pharmaceutically acceptable salt thereof, represents a novel therapeutic and preventative approach to the treatment of a variety of disease states, including, but not limited to, diseases in which bacterial replication is a factor.

The term “Ph” represents a phenyl ring. The terms “Het” or “heterocyclic” as used herein interchangeably, mean a stable heterocyclic ring, that are either saturated or unsaturated, and consist of carbon atoms and from one to three heteroatoms selected from a group consisting of N, O and S, and wherein nitrogen may optionally be oxidized or quaternized, and including any bicyclic group in which any of the above-defined heterocyclic rings is fused to a benzene ring. Ph and Het may be substituted with up to five of C₂₋₆alkyl-, C₁₋₆alkoxy-, R⁴R⁵N(CH₂)₁₋₆—, R⁴R⁵N(CH₂)₂₋₆O—, —CO₂R⁶, —CF₃ or, halogen.

The term “C₁₋₆alkyl” as used herein means a substituted and unsubstituted, straight or branched chain radical of 1 to 6 carbon atoms, unless the chain length is limited thereto, including, but not limited to methyl, ethyl, n-propyl, isopropyl, n-butyl, isobutyl and t-butyl, pentyl, n-pentyl, isopentyl, neopentyl and hexyl and the simple aliphatic isomers thereof. Any C₁₋₆alkyl group may be optionally substituted independently by one or more of OR⁴, R⁴, NR⁴R⁵.

The term “C₃₋₇cycloalkyl” as used herein means substituted or unsubstituted cyclic radicals having 3 to 7 carbons, including but not limited to cyclopropyl, cyclopentyl, cyclohexyl and cycloheptyl radicals.

The term “C₂₋₆alkenyl” as used herein means an alkyl group of 2 to 6 carbons wherein a carbon-carbon single bond is replaced by a carbon-carbon double bond. C₂₋₆alkenyl includes ethylene, 1-propene, 2-propene, 1-butene, 2-butene, isobutene and isomeric pentenes and hexenes. Both cis and trans isomers are included within the scope of this invention. Any C₂₋₆alkenyl group may be optionally substituted independently by one or more of Ph-C₀₋₆alkyl-, Het-C₀₋₆ alkyl-, C₁₋₆alkyl-, C₁₋₆alkoxy-, C₁₋₆mercaptyl-, Ph-C₀₋₆alkoxy-, Het-C₀₋₆alkoxy-, HO—, R⁴R⁵N—, Het-S-C₀₋₆alkyl-, Ph-S—C₀₋₆alkyl-, HO(CH₂)₁₋₆—, R⁴R⁵N(CH₂)₂₋₆—, R⁴R⁵N(CH₂)₂₋₆O—, R⁶CO₂(CH₂)₀₋₆—, R⁶CO₂(CH₂)₁₋₆O—, R⁶SO₂(CH₂)₁₋₆—, —CF₃, —OCF₃, or halogen.

The term “C₂₋₆alkynyl” as used herein means an alkyl group of 2 to 6 carbons wherein one carbon-carbon single bond is replaced by a carbon-carbon triple bond. C₂₋₆alkynyl includes acetylene, 1-propyne, 2-propyne, 1-butyne, 2-butyne, 3-butyne and the simple isomers of pentyne and hexyne.

The term “alkoxy” as used herein means a straight or branched chain radical of 1 to 6 carbon atoms, unless the chain length is limited thereto, bonded to an oxygen atom, including, but not limited to, methoxy, ethoxy, n-propoxy, isopropoxy, and the like.

The term “mercaptyl” as used herein means a straight or branched chain radical of 1 to 6 carbon atoms, unless the chain length is limited thereto, bonded to a sulfur atom, including, but not limited to, methylthio, ethylthio, n-propylthio, isopropylthio, and the like.

The terms “hetero” or “heteroatom” as used herein each mean oxygen, nitrogen and sulfur.

The terms “halo” or “halogen” as used herein each mean F, Cl, Br, and I.

Herein the term C₀ means the absence of the substituent group immediately following; for instance, in the moiety PhC₀₋₆alkyl, when C is 0, the substituent is phenyl.

It will be understood that for compounds of formula (I) and formula (IA), the triazole ring can exist in either of two tautomeric forms as shown in Structure 1. Hydrogen on the triazole ring can exist on either N1 or N3, thus the name for a compound of Structure 1 can be any of the following: 4-(Q)-1H-1,2,3-triazole, 5-(Q)-1H-1,2,3-triazole, 4-(Q)-3H-1,2,3-triazole, 5-(Q)-3H-1,2,3-triazole. These compounds are equivalent and represented herein as one structure and one name (4-(Q)-1H-1,2,3-triazole).

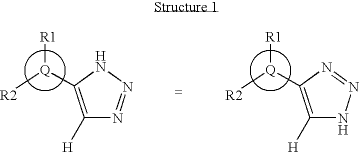

The term “Q” is used herein to represent a 5- or 6-membered monocyclic ring optionally containing up to two heteroatoms selected from N, O, or S, or an 8- to 11-membered fused bicyclic ring optionally containing up to four heteroatoms selected from N, O, or S. A bicyclic ring is defined as two rings that are fused together by two adjacent atoms. Suitably, the ring may be saturated or unsaturated, wherein the nitrogen may optionally be oxidized or quaternized. It will be understood that if Q is a heterocyclic ring, it may be attached to the triazole ring through any heteroatom or carbon atom of Q which results in the creation of a stable structure.

Examples of Q include, but are not limited to phenyl, napthyl, piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, pyridinyl, pyrazinyl, oxazolidinyl, oxazolinyl, oxazolyl, isoxazolyl, morpholinyl, thiazolidinyl, thiazolinyl, thiazolyl, quinuclidinyl, indolyl, quinolinyl, isoquinolinyl, benzimidazolyl, benzopyranyl, benzoxazolyl, furyl, pyranyl, tetrahydrofuryl, tetrahydropyranyl, thienyl, benzoxazolyl, benzofuranyl, benzothiophenyl, thiamorpholinyl sulfoxide, thiamorpholinyl sulfone, oxadiazolyl, triazolyl, thiadiazolyl, oxadiazolyl, isoxazolyl, isothiazolyl, imidazolyl, pyridazinyl, pyrimidinyl and triazinyl which moieties are available commercially or can be made by routine chemical synthesis and are stable.

Suitably, Q is a 5- or 6-membered unsaturated ring or a 9-membered bicyclic ring. For example, Q is thiophene, phenyl, pyridine, benzofuran, or benzo[1,3]dioxole.

It will be understood that for compounds of formula (I), Q is substituted by up to eight of R¹ and if Q is Ph, Q is additionally substituted by one or more R².

It will be understood that for compounds of this invention, Q is substituted by up to eight substituents, selected independently from R¹ and R².

Suitably, R¹ is H—, Ph-C₀₋₆alkyl-, Het-C₀₋₆ alkyl-, C₁₋₆alkyl-, C₁₋₆alkoxy-, C₁₋₆mercaptyl-, Ph-C₀₋₆alkoxy-, Het-C₀₋₆alkoxy-, HO—, R⁴R⁵N—, Het-S—C₀₋₆alkyl-, Ph-S—C₀₋₆alkyl-, HO(CH₂)₁₋₆—, R⁴R⁵N(CH₂)₂₋₆—, R⁴R⁵N(CH₂)₂₋₆O—, R⁶CO₂(CH₂)₀₋₆—, R⁶CO₂(CH₂)₁₋₆O—, R⁶SO₂(CH₂)₁₋₆—, —CF₃, —OCF₃, or halogen, and Ph or Het are substituted with up to five of C₂₋₆alkyl-, C₁₋₆alkoxy-, R⁴R⁵N(CH₂)₁₋₆—, R⁴R⁵N(CH₂)₂₋₆O—, —CO₂R⁶, —CF₃ or, halogen. For example, R¹ is halogen, C₁₋₆alkyl-, C₁₋₆alkoxy-, or —OH. For example, R¹ is bromine, chlorine, methyl, ethyl, methoxyl, or hydroxyl.

Suitably, R² is Ph-C₀₋₆alkyl-, Het-C₀₋₆ alkyl-, C₅₋₆alkyl-, C₂₋₆alkoxy-, C₁₋₆-mercaptyl-, Ph-C₀₋₆alkoxy-, Het-C₀₋₆alkoxy-, HO—, R⁴R⁵N—, Het-S—C₀₋₆alkyl-, Ph-S—C₀₋₆alkyl-, HO(CH₂)₁₋₆—, R⁴R⁵N(CH₂)₂₋₆—, R⁴R⁵N(CH₂)₂₋₆O—, R⁶CO₂(CH₂)₀₋₆—, R⁶CO₂(CH₂)₁₋₆O—, R⁶SO₂(CH₂)₁₋₆—, —CF₃ or —OCF₃, and Ph or Het are substituted with up to five of C₂₋₆alkyl-, C₁₋₆alkoxy-, R⁴R⁵N(CH₂)₁₋₆—, R⁴R⁵N(CH₂)₂₋₆O—, —CO₂R⁶, —CF₃ or, halogen; wherein R⁴, R⁵, and R⁶ are independently selected from H, C₂₋₆alkyl, C₃₋₆alkenyl, C₃₋₆alkynyl, Ph-C₀₋₆alkyl, Het-C₀₋₆alkyl, or C₃₋₇cycloalkyl-C₀₋₆alkyl. For example, R² is —NR⁴R⁵, —CF₃, Ph-S—C₀₋₆alkyl-, Ph-C₀₋₆alkoxy-. For example, R² is benzylamine, propylamine, furan-3-ylmethylamine, furan-2-ylmethylamine, —CF₃, Ph-CH₂—O—, (4-Cl)Ph-S—.

For compounds of formula IA, R³ is suitably H—, halogen, or R³ and Q together form a fused bicyclic or tricyclic saturated or unsaturated ring system wherein R³ is —C—, or —C═C—. For example, R³ is hydrogen, bromine, or is fused to Q by —C— to form a dihydro-indenotriazole or by —C═C— to form a napthotriazole or an acetonapthotriazole.

Suitably, R⁴, R⁵, and R⁶ are independently selected from H—, C₂₋₆alkyl-, C₃₋₆alkenyl-, C₃₋₆alkynyl-, Ph-C₀₋₆alkyl-, Het-C₀₋₆alkyl-, or C₃₋₇cycloalkyl-C₀₋₆alkyl-. For example R⁴, R⁵, and R⁶ are independently selected hydrogen, benzyl, furanyl, and propyl.

Further, it will be understood that when a moiety is “optionally substituted” the moiety may have one or more optional substituents, each optional substituent being independently selected.

Suitably, pharmaceutically acceptable salts of formula (I) include, but are not limited to, salts with inorganic acids such as hydrochloride, sulfate, phosphate, diphosphate, hydrobromide, and nitrate, or salts with an organic acid such as malate, maleate, fumarate, tartrate, succinate, citrate, acetate, lactate, methanesulfonate, p-toluenesulfonate, palmitate, salicylate, and stearate.

The compounds of the present invention may contain one or more asymmetric carbon atoms and may exist in racemic and optically active forms. The stereocenters may be (R), (S) or any combination of R and S configuration, for example, (R,R), (R,S), (S,S) or (S,R). All of these compounds are within the scope of the present invention.

Novel intermediates useful in making compounds of this invention are as follows:
4-ethynyl-benzo[1,3]dioxole;
1-(4-chloro-phenylsulfanyl)-2-ethynylbenzene;
(3-phenyl-propyl)-(3-ethynylphenyl)amine;
phenethyl-(3-ethynylphenyl)-amine;
furan-2-ylmethyl-(3-ethynylphenyl)-amine;
furan-3-ylmethyl-(3-ethynylphenyl)-amine;
napthalene-1-ylmethyl-(3-ethynylphenyl)-amine; and
napthalene-2-ylmethyl-(3-ethynylphenyl)-amine.

The intermediates useful for this invention were made according to the Schemes herein.

Among the compounds of the formula (IA) are the following compounds:
3-(1H-1,2,3-triazol-4-yl)-phenol;
4-(3-iodophenyl)-1H-1,2,3-triazole;
4-(2-fluorophenyl)-1H-1,2,3-triazole;
4-(4-n-butylphenyl)-1H-1,2,3-triazole;
4-(2-chlorophenyl)-1H-1,2,3-triazole;
N-(3-[1H-1,2,3-triazol-4-yl]phenyl)benzamide;
3-(1H-1,2,3-triazol-4-yl)-phenylamine;
N-(3-[1H-1,2,3-triazol-4-yl]phenyl)acetamide;
4-(4-trifluoromethylphenyl)-1H-1,2,3-triazole;
4-(3-trifluoromethylphenyl)-1H-1,2,3-triazole;
4-(4-n-propylphenyl)-1H-1,2,3-triazole;
4-(4-methoxyphenyl)-1H-1,2,3-triazole;
4-(3-methylphenyl)-1H-1,2,3-triazole;
2-(1H-1,2,3-triazol-4-yl)-pyridine;
4-(4-chlorophenyl)-1H-1,2,3-triazole;
4-(4-ethylphenyl)-1H-1,2,3-triazole;
4-(1H-1,2,3-triazolyl)-phenylamine;
4-(4-methylphenyl)-1H-1,2,3-triazole;
2-(1H-1,2,3-triazolyl)-5-methylpyridine;
2-(1H-1,2,3-triazol-4-yl)-4-methyl-pyridine;
1-(1H-1,2,3-triazolyl)cyclohexanol;
4-(thiophen-2-yl)-1H-1,2,3-triazole;
4-(thiophen-3-yl)-1H-1,2,3-triazole;
4-(2-methylphenyl)-1H-1,2,3-triazole;
4-(1,3-dimethylphenyl)-1H-1,2,3-triazole;
4-(4-bromophenyl)-1H-1,2,3-triazole;
4-(1,3-dichlorophenyl)-1H-1,2,3-triazole;
4-(1-biphenyl-2-yl)-1H-1,2,3-triazole;
4-(2-benzyloxy-phenyl)-1H-1,2,3-triazole;
2-(1H-1,2,3-triazol-4-yl)-6-methylpyridine;
3-(1H-1,2,3-triazol-4-yl)-pyridine;
4-(1H-1,2,3-triazol-4-yl)-pyridine;
4-(2-methoxyphenyl)-1H-1,2,3-triazole;
4-(2-bromophenyl)-1H-1,2,3-triazole;
4-benzo[1,3]dioxol-5-yl-1H-1,2,3-triazole;
2-(1H-1,2,3-triazol-4-yl)-benzofuran;
4-benzo[1,3]dioxol-4-yl-1H-1,2,3-triazole;
4-(2-[4-chloro-phenylsulfanyl]-phenyl)-1H-1,2,3-triazole;
(3-phenyl-propyl)-(3-[1H-1,2,3-triazol-4-yl]phenyl)amine;
phenethyl-(3-[1H-1,2,3-triazol-4-yl]phenyl)amine;
furan-2-ylmethyl-(3-[1H-1,2,3-triazol-4-yl]phenyl)amine;
furan-3-ylmethyl-(3-[1H-1,2,3-triazol-4-yl]phenyl)amine;
napthalene-1-ylmethyl-(3-[1H-1,2,3-triazol-4-yl]phenyl)amine;
napthalene-2-ylmethyl-(3-[1H-1,2,3-triazol-4-yl]phenyl)amine;
4-(1H-1,2,3-triazol-4-yl)-phenol;
benzyl-(3-[1H-1,2,3-triazol-4-yl]phenyl)amine;
4-(4-fluorophenyl)-1H-1,2,3-triazole;
2-bromo-(1H-1,2,3-triazol-4-yl)-phenol;
2,6-dibromo-5-(1H-1,2,3-triazol-4-yl)-phenol;
2,4-dibromo-5-(1H-1,2,3-triazolyl)-phenol;
2-(5-bromo-1H-1,2,3-triazol-4-yl)-4-methyl-pyridine;
1H-naptho[1,2-d]-1,2,3-triazole;
2,8-dihydro-indeno[1,2-d]-1,2,3-triazole;
4-phenyl-1H-1,2,3-triazole; and
5,5a,6,8-tetrahydro-4H-acenaphtho[4,5-d]-1,2,3-triazole.

Among the most compounds of the formula (IA) are the following compounds:
4-(3-iodophenyl)-1H-1,2,3-triazole;
4-(2-fluorophenyl)-1H-1,2,3-triazole;
4-(2-chlorophenyl)-1H-1,2,3-triazole;
4-(3-methylphenyl)-1H-1,2,3-triazole;
4-(4-chlorophenyl)-1H-1,2,3-triazole;
4-(4-ethylphenyl)-1H-1,2,3-triazole;
4-(4-methylphenyl)-1H-1,2,3-triazole;
2-(1H-1,2,3-triazol-4-yl)-5-methylpyridine;
2-(1H-1,2,3-triazol-4-yl)-4-methyl-pyridine;
4-(thiophen-3-yl)-1H-1,2,3-triazole;
4-(4-bromophenyl)-1H-1,2,3-triazole;
4-(1,3-dichlorophenyl)-1H-1,2,3-triazole;
2-(1H-1,2,3-triazol-4-yl)-benzofuran;
furan-2-ylmethyl-(3-[1H-1,2,3-triazol-4-yl]phenyl)amine;
furan-3-ylmethyl-(3-[1H-1,2,3-triazol-4-yl]phenyl)amine;
benzyl-(3-[1H-1,2,3-triazol-4-yl]phenyl)amine;
4-(4-fluorophenyl)-1H-1,2,3-triazole;
2-bromo-5-(1H-1,2,3-triazol-4-yl)-phenol;
2,4-dibromo-5-(1H-1,2,3-triazol-4-yl)-phenol; and
2-(5-bromo-1H-1,2,3-triazol-4-yl)-4-methyl-pyridine.
   Methods of Preparation

Compounds of the formulae (I) or (IA), were prepared by methods analogous to those described in Scheme 1.

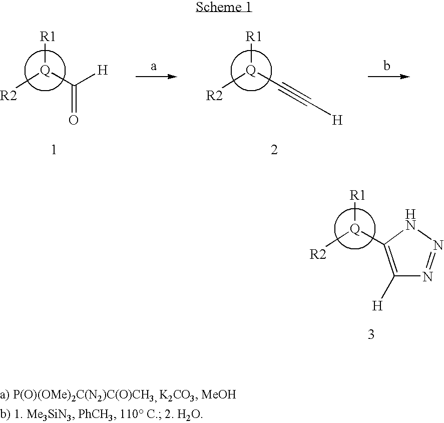

An aldehyde (such as 2-thiophenecarboxaldehyde) (1-Scheme1) was treated with 1-diazo-2-oxopropylphosphonate and potassium carbonate in dry methanol to provide 2-Scheme1. Treatment of the acetylene (such as 2-ethynylthiophene) (2-Scheme1) with azidotrimethylsilane in refluxing toluene, followed by addition of water afforded 3-Scheme1.

Compounds of the formulae (I) or (IA), were R² is NHR⁴ were prepared by methods analogous to those described in Scheme 2.

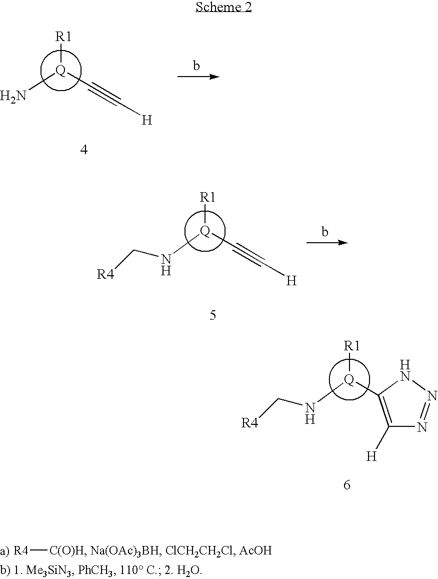

An alkynyl aniline (such as 3-ethynylphenylamine) was substituted by a reductive amination reaction with an aldehyde to provide 5-Scheme2. Treatment of the acetylene (5-Scheme2) with azidotrimethylsilane in refluxing toluene, followed by addition of water afforded 6-Scheme2.

Formulation of Pharmaceutical Compositions

Pharmaceutically effective compounds of this invention (and pharmaceutically acceptable salts thereof) may be administered in conventional dosage forms prepared by, for example, combining a compound of this invention of formula (I) or (IA) (“active ingredient”) in an amount sufficient to treat diseases in which bacterial replication is a factor (“MetAP-mediated disease states”) with standard pharmaceutical carriers or diluents according to conventional procedures well known in the art. These procedures may involve mixing, granulating and compressing or dissolving the ingredients as appropriate to the desired preparation.

The pharmaceutical carrier employed may be, for example, either a solid or liquid. Exemplary of solid carriers are lactose, terra alba, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time delay material well known to the art, such as glyceryl monostearate or glyceryl distearate alone or with a wax.

A wide variety of pharmaceutical forms can be employed. Thus, if a solid carrier is used, a preparation may be tableted, placed in a hard gelatin capsule in powder or pellet form or in the form of a troche or lozenge. An amount of solid carrier may vary widely but may be from about 25 mg to about 1000 mg. When a liquid carrier is used, a preparation may be in the form of a syrup, emulsion, soft gelatin capsule, sterile injectable liquid such as an ampule or nonaqueous liquid suspension.

An active ingredient may also be administered topically to a mammal in need of treatment or prophylaxis of MetAP-mediated disease states. An amount of active ingredient required for therapeutic effect on topical administration may vary with a compound chosen, nature and severity of a disease state being treated and the mammal undergoing treatment, and may ultimately be at the discretion of a physician. A suitable dose of an active ingredient may be 1.5 mg to 500 mg for topical administration, an exemplary dosage being 1 mg to 100 mg, for example 5 to 25 mg administered two or three times daily.

By topical administration is meant non-systemic administration and may include an application of an active ingredient externally to epidermis, to the buccal cavity, instillation of such a compound into the ear, eye or nose, or where a compound does not significantly enter the blood stream. By systemic administration is meant oral, intravenous, intraperitoneal and intramuscular administration, among others.

While it is possible for an active ingredient to be administered alone as a raw chemical, it may also be present as a pharmaceutical formulation. An active ingredient may comprise, for topical administration, from 0.001% to 10% w/w, e.g. from 1% to 2% by weight of the formulation although it may comprise as much as 10% w/w but in certain embodiments will not be excess of 5% w/w and in other embodiments will range from 0.1% to 1% w/w of the formulation.

Topical formulations of the present invention, both for veterinary and for human medical use, may comprise an active ingredient together with one or more acceptable carrier(s) therefor and optionally any other therapeutic ingredient(s). Exemplary carrier(s) are ‘acceptable’ in the sense of being compatible with the other ingredients of the formulation and not deleterious to the recipient thereof.

Formulations suitable for topical administration include, but are not limited to, liquid or semi-liquid preparations suitable for penetration through the skin to a site of inflammation, such as liniments, lotions, creams, ointments or pastes, and drops suitable for administration to the eye, ear or nose, among others.

Drops according to the present invention may comprise sterile aqueous or oily solutions or suspensions and may be prepared by dissolving the active ingredient in a suitable aqueous or alcoholic solution of a bactericidal and/or fungicidal agent and/or any other suitable preservative, and may, for example, include a surface active agent. A resulting solution may then be clarified by filtration, transferred to a suitable container which is then sealed and sterilized by autoclaving or maintaining at 98-100° C. for half an hour, among other ways. Alternatively, a solution may be sterilized by filtration and transferred to a container by an aseptic technique. Examples of bactericidal and fungicidal agents suitable for inclusion in drops may comprise phenylmercuric nitrate or acetate (0.002%), benzalkonium chloride (0.01%) and chlorhexidine acetate (0.01%). Suitable solvents for preparation of an oily solution may include glycerol, diluted alcohol and propylene glycol.

Lotions according to the present invention include, but are not limited to, those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil, among others.

Creams, ointments or pastes according to the present invention may be semi-solid formulations of an active ingredient for external application. They may be made by mixing an active ingredient in finely divided or powdered form, alone or in solution or suspension in an aqueous or non-aqueous fluid, with the aid of suitable machinery, with a greasy or non-greasy basis. A basis may comprise hydrocarbons, such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol. A formulation may incorporate any suitable surface-active agent such as an anionic, cationic or non-ionic surfactant such as esters or polyoxyethylene derivatives thereof. Suspending agents such as natural gums, cellulose derivatives or inorganic materials such as silicaceous silicas, and other ingredients such as lanolin, may also be included.

An active ingredient may also be administered by inhalation. By “inhalation” is meant intranasal or oral inhalation administration. Appropriate dosage forms for such administration, such as an aerosol formulation or a metered dose inhaler, may be prepared by conventional techniques. A daily dosage amount of an active ingredient administered by inhalation is from about 0.1 mg to about 100 mg per day, for example about 1 mg to about 10 mg per day.

By the term “treating” is meant either prophylactic or therapeutic therapy. Such compound may be administered to such mammal in a conventional dosage form prepared by combining the compound of this invention with a conventional pharmaceutically acceptable carrier or diluent according to known techniques. It will be recognized by one of skill in the art that the form and character of the pharmaceutically acceptable carrier or diluent may be dictated by the amount of active ingredient with which it is to be combined, the route of administration and other well-known variables. The compound is administered to a mammal in need of treatment for diseases in which bacterial replication is a factor, in an amount sufficient to decrease or eliminate symptoms associated with these disease states. The route of administration may be oral or parenteral, among others.

The term parenteral as used herein includes, but is not limited to, intravenous, intramuscular, subcutaneous, intra-rectal, intravaginal or intraperitoneal administration. A daily parenteral dosage regimen may for example be from about 30 mg to about 300 mg per day of active ingredient. The daily oral dosage regimen may, for example, be from about 100 mg to about 2000 mg per day of active ingredient.

It will be recognized by one of skill in the art that a quantity and spacing of individual dosages of a compound of this invention may be determined by the nature and extent of a condition being treated, a form, route and site of administration, and mammal being treated, and that such quantity and spacing may be determined by conventional techniques. It will also be appreciated by one of skill in the art that an exemplary course of treatment, i.e., the number of doses of a compound given per day for a defined number of days, may be ascertained by those skilled in the art using conventional course of treatment determination tests.

Definitions

Herein, the terms “a” and “an” mean “one or more” when used in this application, including the claims.

The invention further provides for homologues, co-complexes, mutants and derivatives of a MetAP crystal structure of the invention.

The term “co-complex” and “cocrystal” each mean a MetAP or a mutant or homologue of a MetAP in covalent or non-covalent association with a chemical entity or compound.

As used herein, the terms “antagonist” and “inhibitor” as herein mean an agent that (i) decreases or inhibits an activity of a MetAP gene or protein or (ii) decreases or inhibits an activity of a gene or polypeptide encoded by a gene that is up- or down-regulated by a MetAP polypeptide.

As used herein, the term “active site” refers to a region of a MetAP binding pocket where a molecule binds and catalysis takes place. It may be comprised and bound by amino acid residues that are in contact with a substrate or that interact with a substrate(s) through water molecules or amino acids that, although not being in contact with a substrate(s), nonetheless allow certain positioning of amino acids that are in contact and that without certain positioning they would not be able to interact in a way conducent to catalysis with a substrate(s). These interactions between amino acids and substrate(s) may be responsible for binding of a substrate to MetAP, for certain positioning of a substrate for catalysis, and for stabilization of any reaction intermediates and for binding or release of a product from an active site. An active site may also be comprised of amino acids that are responsible for catalysis. These amino acids interact with a substrate(s) through hydrogen bonds or by close proximity to electron-donor or electron-acceptor centers in a substrate. These amino acids may act themselves as electron-donor or electron-acceptor centers for catalysis to take place.

As used herein, the term “biological activity” or “activity” means (i) any observable effect flowing from an interaction between an enzyme or polypeptide and a modulator, (ii) transcription regulation, modulator binding, and polypeptide binding, (iii) an interaction or association between (1) a compound and an enzyme, for example, a bacterial methionine aminopeptidase, or (2) a component of a complex comprising a compound and an enzyme, for example, a bacterial methionine aminopeptidase, or (3) a compound and a subunit(s) or a cofactor(s) of an enzyme, for example, a bacterial methionine aminopeptidase, or (iv) active site catalysis of an enzyme, for example, a bacterial methionine aminopeptidase, or (vi) a chemical reaction carried out by or correlated with an enzyme, for example, a bacterial methionine aminopeptidase.

As used herein, the terms “candidate substance” and “candidate compound” are used interchangeably and refer to a substance that is believed to interact with another moiety, for example an modulator that is believed to interact with a complete, or a fragment of, an enzyme, such as a MetAP polypeptide, and which can be evaluated for such an interaction. Representative candidate substances or compounds include, but are not limited to, xenobiotics such as drugs and other therapeutic agents, carcinogens and environmental pollutants, natural products and extracts, as well as endobiotics such as glucocorticosteroids, steroids, fatty acids and prostaglandins. Other examples of candidate compounds that can be investigated using methods of the present invention include, but are not restricted to, agonists and antagonists of a MetAP polypeptide, toxins and venoms, viral epitopes, hormones (e.g., glucocorticosteroids, opioid peptides, steroids, etc.), hormone receptors, peptides, enzymes, enzyme substrates, co-factors, lectins, sugars, oligonucleotides or nucleic acids, oligosaccharides, proteins, small molecules and monoclonal antibodies.

As used herein, the terms “cells” or “host organism” are used interchangeably and mean not only to a particular subject cell, but also to any progeny or potential progeny of such a cell. Because certain modifications can occur in succeeding generations due to either mutation or environmental influences, such progeny might not, in fact, be identical to the parent cell, but are still included within the scope of the term as used herein.

“Bacteria(al)” means a (i) prokaryote, including but not limited to, a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma, and further including, but not limited to, a member of the species or group, Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrheae, Neisseria meningitidis, Staphylococcus aureus, Staphylococcus epidermidis, Corynebacterium diptheriae, Gardnerella vaginalis, Mycobacterium tuberculosis, Mycobacterium bovis, Mycobacterium ulcerans, Mycobacterium leprae, Actinomyctes israelii, Listeria monocytogenes, Bordetella pertusis, Bordatella parapertusis, Bordetella bronchiseptica, Escherichia coli, Shigella dysenteriae, Haemophilus influenzae, Haemophilus aegyptius, Haemophilus parainfluenzae, Haemophilus ducreyi, Bordetella, Salmonella typhi, Citrobacter freundii, Proteus mirabilis, Proteus vulgaris, Yersinia pestis, Kleibsiella pneumoniae, Serratia marcessens, Serratia liquefaciens, Vibrio cholera, Shigella dysenteii, Shigella flexneri, Pseudomonas aeruginosa, Franscisella tularensis, Brucella abortis, Bacillus anthracis, Bacillus cereus, Clostridium perfringens, Clostridium tetani, Clostridium botulinum, Treponema pallidum, Rickettsia rickettsi and Chlamydia trachomitis, and (ii) an archaeon, including but not limited to Archaebacter.

As used herein, the term “detecting” means confirming presence of a target entity by observing an occurrence of a detectable signal, such as a radiologic or spectroscopic signal that will appear exclusively in the presence of the target entity.

As used herein, the term “expression” generally refers to the cellular processes by which a biologically active polypeptide is produced.

As used herein, the term “gene” is used for simplicity to refer to a functional protein, polypeptide or peptide encoding unit. As will be understood by those in the art, this functional term includes both genomic sequences and cDNA sequences.

As used herein, the term “crystal lattice” means an array of points defined by vertices of packed unit cells.

As used herein, “hexagonal unit cell” means a unit cell wherein a=b≠c; and α=β=90, γ=120°. The vectors a, b, and c describe unit cell edges and angles α,β, and γ describe unit cell angles. In an embodiment of the present invention, for example S. pneumoniae, a unit cell has lattice constants of, a=56.77, b=69.16, c=80.51 Ångstroms. While certain lattice constants are provided, a crystalline polypeptide of the present invention also comprises variations from certain lattice constants, wherein variations range from about one to about two percent.

As used herein, the term “hybridization” means binding of a probe molecule, a molecule to which a detectable moiety has been bound, to a target sample.

As used herein, the term “interact” means detectable interactions between molecules, such as can be detected using, for example, a yeast two hybrid assay. The term “interact” is also meant to include “binding” interactions between molecules. Interactions can, for example, be protein-protein or protein-nucleic acid in nature.

As used herein, the term “isolated” means oligonucleotides substantially free of other nucleic acids, proteins, lipids, carbohydrates or other materials with which they may be associated, such association being either in cellular material or in a synthesis medium. The term can also be applied to polypeptides, in which case the polypeptide will be substantially free of nucleic acids, carbohydrates, lipids and other undesired polypeptides.

As used herein, the term “labeled” means attachment of a moiety, capable of detection by spectroscopic, radiologic or other methods, to a probe molecule.

As used herein, the term “modified” means an alteration from an entity's normally occurring state. An entity can be modified by removing discrete chemical units or by adding discrete chemical units. The term “modified” encompasses detectable labels as well as those entities added as aids in purification.

“Modulation,” “modulating,” to “modulate” means with reference to a mechanism of action, activity, enzyme activity, enzyme, polynucleotide, crystal or coordinate herein (i) altering, modulating, raising, enhancing, increasing, lowering, diminishing, preventing or stopping an activity or activities of an enzyme, for example, a bacterial methionine aminopeptidase, or (ii) enhancing, improving, or stabilizing an interaction or association between (1) a compound and an enzyme, for example, a bacterial methionine aminopeptidase, or (2) a component of a complex comprising a compound and an enzyme, for example, a bacterial methionine aminopeptidase, or (3) a compound and subunit(s) or cofactor(s) of an enzyme, for example, a bacterial methionine aminopeptidase, or (iii) altering, modulating, lowering, diminishing, preventing or stopping an active site activity of an enzyme, for example, a bacterial methionine aminopeptidase, or (iv) up-regulation (i.e., activation or stimulation) and down-regulation (i.e. inhibition or suppression) of an activity of an enzyme, for example, a bacterial methionine aminopeptidase.

“Modulator(s)” means a compound or composition that causes, affects, or correlates with, modulation, modulating, or may modulate through cause, affect or correlation.

As used herein, the term “molecular replacement” means a method that involves generating a preliminary model of a wild-type MetAP ligand binding domain, or a MetAP mutant crystal whose structure coordinates are unknown, by orienting and positioning a molecule or model whose structure coordinates are known within a unit cell of the unknown crystal so as best to account for an observed diffraction pattern of the unknown crystal. Phases can then be calculated from this model and combined with observed amplitudes to give an approximate Fourier synthesis of a structure whose coordinates are unknown. This, in turn, may be subject to any of the several forms of refinement to provide a final, accurate structure of an unknown crystal. See, e.g., Lattman, (1985) Method Enzymol., 115: 55-77; Rossmann, ed, (1972) The Molecular Replacement Method, Gordon & Breach, New York. Using the structure coordinates of an active site of MetAP provided by this invention, molecular replacement may be used to determine the structure coordinates of a crystalline mutant or homologue of an MetAP active site, or of a different crystal form of an MetAP active site.

As used herein, the term “polypeptide” means any polymer comprising any of the 20 protein amino acids, regardless of its size. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted. As used herein, the terms “protein”, “polypeptide” and “peptide” are used interchangeably herein when referring to a gene product.

As used herein, the terms “structure coordinates” and “structural coordinates” mean mathematical or spatial coordinates derived from mathematical equations related to patterns obtained on diffraction of a monochromatic beam of X-rays by atoms (scattering centers) of a molecule in crystal form. The diffraction data may be used to calculate an electron density map of a repeating unit of a crystal. Electron density maps may be used to establish positions of individual atoms within a unit cell of a crystal.

Those of skill in the art understand that a set of coordinates determined by X-ray crystallography is not without standard error. An error in assigned coordinates may become reduced as resolution is increased, since more experimental diffraction data may available for model fitting and refinement. Thus, for example, more diffraction data may be collected from a crystal that diffracts to a resolution of 2.8 angstroms than from a crystal that diffracts to a lower resolution, such as 3.5 angstroms. Consequently, refined structural coordinates may be more accurate when fitted and refined using data from a crystal that diffracts to higher resolution. The design of agonists, antagonists, and modulators for MetAP depends on the accuracy of the structural coordinates. If the coordinates are not sufficiently accurate, then a design process may be ineffective. In certain cases, it may be difficult or impossible to collect sufficient diffraction data to define atomic coordinates precisely when a crystal diffracts to a resolution of 3.5 angstroms or poorer. Thus, in certain cases, it may be difficult to use X-ray structures in structure-based agonist and antagonist design when X-ray structures are based on crystals that diffract to a resolution of 3.5 angstroms or poorer. However, crystals diffracting to 2.8 angstroms or better may yield X-ray structures with an accuracy enabling structure-based drug design. Further improvement in resolution may further facilitate structure-based design, but the coordinates obtained at 2.8 angstroms resolution may be adequate for certain purposes.

Also, those of skill in the art will understand that MetAP proteins may adopt different conformations when different agonists, antagonists, and modulators are bound. Subtle variations in a conformation may also occur when different agonists are bound, and when different antagonists are bound. Structure-based design of MetAP modulators may depend to some degree on a knowledge of differences in conformation that occur when agonists and antagonists are bound. Thus, structure-based modulator design may be facilitated by an availability of X-ray structures of complexes with agonists as well as antagonists.

As used herein, the term “substantially pure” means that a polynucleotide or polypeptide is substantially free of the sequences and molecules with which it is or may be associated in its natural state or synthetic state, and those molecules used in an isolation procedure. The term “substantially free” means that a sample is at least 50%, or may be at least 70%, or may also be at least 80% or at least 90% free of materials and compounds with which it is or may be associated in nature.

As used herein, the term “transcription” means a process involving an interaction of an RNA polymerase with a gene that directs expression of RNA. The process includes, but is not limited to the following steps: (a) transcription initiation, (b) transcript elongation, (c) transcript splicing, (d) transcript capping, (e) transcript termination, (f) transcript polyadenylation, (g) nuclear export of a transcript, (h) transcript editing, and (i) stabilizing a transcript.

As used herein, the term “unit cell” means a basic parallelipiped shaped block. A volume of a crystal may be constructed by regular assembly of such blocks. Each unit cell may comprise a complete representation of a unit of pattern, any repetition of which builds up a crystal. Thus, the term “unit cell” means a fundamental portion of a crystal structure that may be repeated infinitely by translation in three dimensions. A unit cell may be characterized by three vectors a, b, and c, not colocated in a plane, which form the edges of a parallelepiped. Angles α, β, and γ define angles between the vectors: angle α is an angle between vectors b and c; angle β is an angle between vectors a and c; and angle γ is an angle between vectors a and b. The volume of a crystal may be constructed by regular assembly of unit cells; each unit cell comprises a complete representation of a unit of pattern, any repetition of which builds up a crystal.

As used herein, the term “mutant” or “mutation” carries its traditional connotation and means a change, inherited, naturally occurring or introduced, in a nucleic acid or polypeptide sequence, and is used in its sense as generally known to those of skill in the art. For example, a MetAP polypeptide, i.e., a polypeptide displaying a biological activity of wild-type MetAP activity, characterized by a replacement of an active-site amino acid from a wild-type prenyltransferase sequence.

MetAP mutants may also be generated by site-specific incorporation of unnatural amino acids into a MetAP protein using biosynthetic methods of C. J. Noren et al, Science, 244:182-188 (1989), among other methods. In this method, a codon encoding an amino acid of interest in wild-type MetAP is replaced by a “blank” nonsense codon, TAG, using oligonucleotide-directed mutagenesis. A suppressor directed against this codon is then chemically aminoacylated in vitro with a desired unnatural amino acid. The aminoacylated residue is then added to an in vitro translation system to yield a mutant MetAP with a site-specific Incorporated unnatural amino acid.

Selenocysteine or selenomethionine may be incorporated into wild-type or mutant metallo MetAP by expression of MetAP-encoding cDNAs in auxotrophic E. coli strains (W. A. Hendrickson et al, EMBO J., 9(5):1665-1672 (1990)) or a normal strain grown in a medium supplemented with appropriate nutrients that will prevent endogenous synthesis of methionine. In either of these methods, the wild-type or mutated MetAP cDNA may be expressed in a host organism on a growth medium depleted of either natural cysteine or methionine (or both) but enriched in selenocysteine or selenomethionine (or both).

The term “heavy atom derivative” refers to derivatives of MetAP produced by chemically modifying a crystal of MetAP. A native crystal may be treated by immersing it in a solution containing a desired metal salt, or organometallic compound, e.g., lead chloride, gold thiomalate, thimerosal or uranyl acetate, which upon diffusion into a protein crystal may bind to the protein. The location of the bound heavy metal atom site(s) may be determined by X-ray diffraction analysis of the treated crystal. This information may be used to generate phase angle information needed to construct a three-dimensional electron density map from which a model of an atomic structure of an enzyme may be derived (T. L Blundel and N. L. Johnson, Protein Crystallography, Academic Press (1976)).

The term “space group” refers to an arrangement of symmetry elements (i.e. molecules) throughout a crystal. There are 132 possible arrangements, each one identified by a symbol. The space group symbol is denoted by a letter (P, F, I, C) and numbers with or without subscripts, for example: P2₁, I222, C2₁2₁2₁, etc.

Methods of Identifying Inhibitors of MetAP from Streptococcus pneumoniae Crystalline Structure

An aspect of this invention involves a method for identifying inhibitors of a MetAP characterized by a crystal structure and an active site described herein, and crystal structures of complexes with its substrates. An exemplary crystalline structure of the invention permits identification of inhibitors of methionine aminopeptidase activity. Such inhibitors may be competitive, binding to all or a portion of an active site of MetAP; or non-competitive and bind to and inhibit methionine aminopeptidase whether or not it is bound to another chemical entity.

One design approach is to probe a MetAP crystal of the invention with molecules composed of a variety of different chemical entities to determine sites for interaction between candidate MetAP inhibitors and an enzyme. For example, high resolution X-ray diffraction data collected from crystals saturated with solvent allows the determination of where each type of solvent molecule binds. Small molecules that bind tightly to those sites may then be designed and synthesized and tested for a MetAP inhibitor activity (J. Travis, Science, 262:1374 (1993)).

This invention also enables the development of compounds that may isomerize to short-lived reaction intermediates in a chemical reaction of a substrate or other compound that binds to or with an exemplary MetAP. Thus, time-dependent analysis of structural changes in a MetAP during its interaction with other molecules may be permitted. Reaction intermediates of a MetAP can also be deduced from a reaction product in co-complex with a MetAP. Such information may be useful to design improved analogues of known MetAP inhibitors or to design novel classes of inhibitors based on reaction intermediates of a MetAP enzyme and MetAP inhibitor co-complex. This provides a route for designing MetAP inhibitors with both specificity and stability.

Another approach made possible by this invention, is to screen computationally small molecule data bases for elements or compounds that may bind in whole, or in part, to a MetAP enzyme. In this screening, the quality of fit of such entities or compounds to the binding site may be judged either by shape complementarity or by estimated interaction energy (E. C. Meng et al, J. Comp. Chem., 13:505-524 (1992)).

Because MetAP may crystallize in more than one crystal form, the structure coordinates of MetAP, or portions thereof, as provided by this invention are particularly useful to solve a structure of those other crystal forms of MetAP. They may also be used to solve a structure of MetAP mutant co-complexes, or of a crystalline form of any other protein with significant amino acid sequence homology to any functional domain of MetAP.

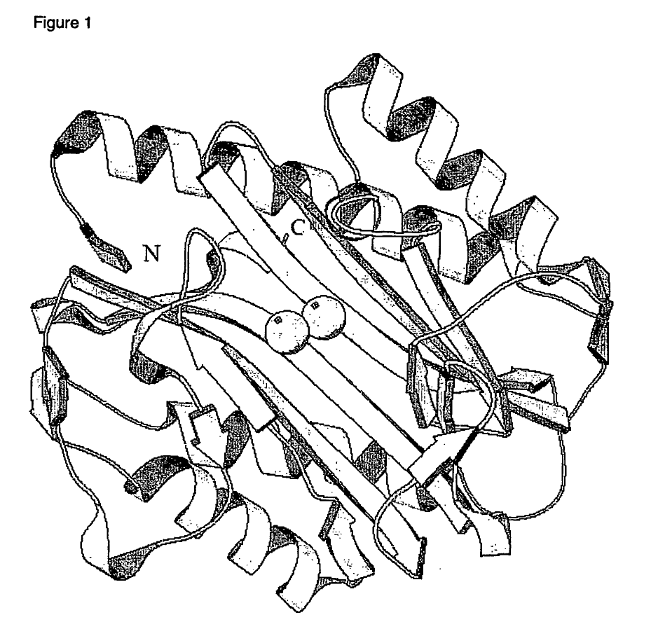

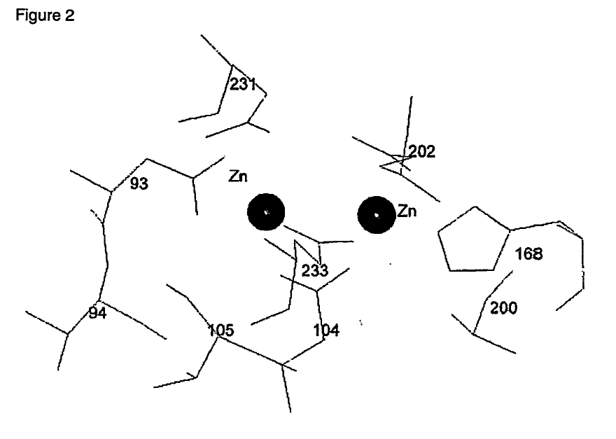

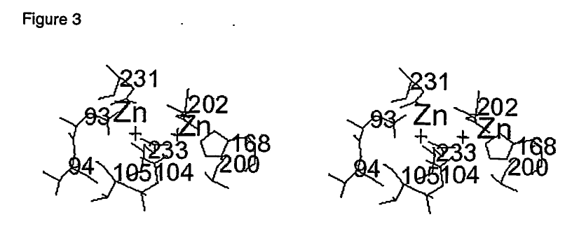

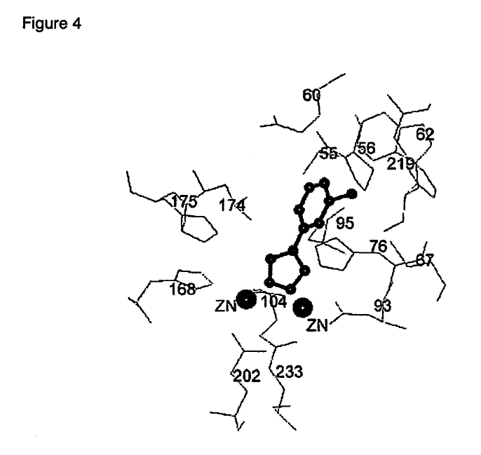

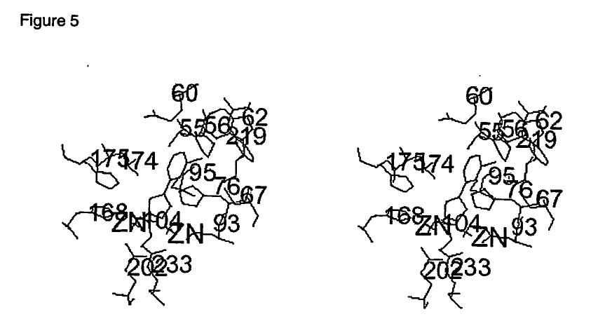

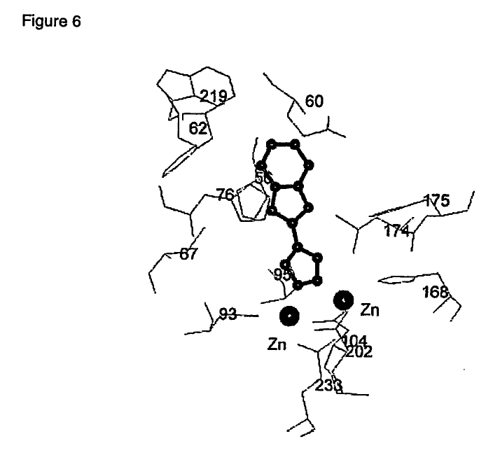

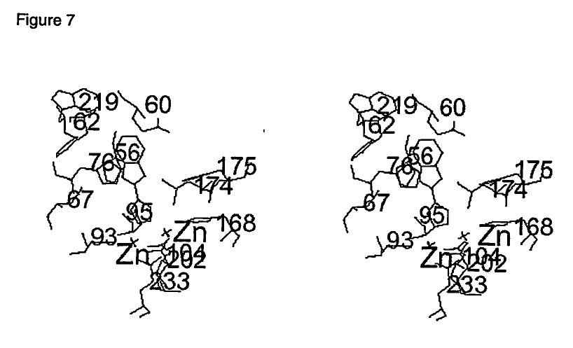

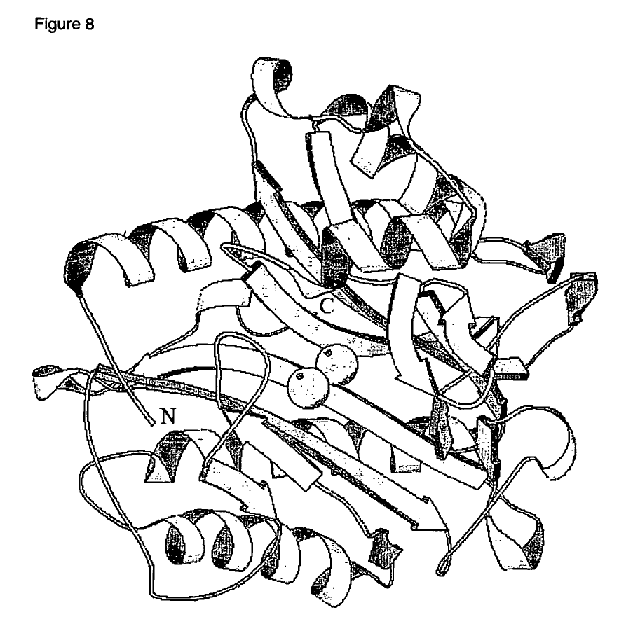

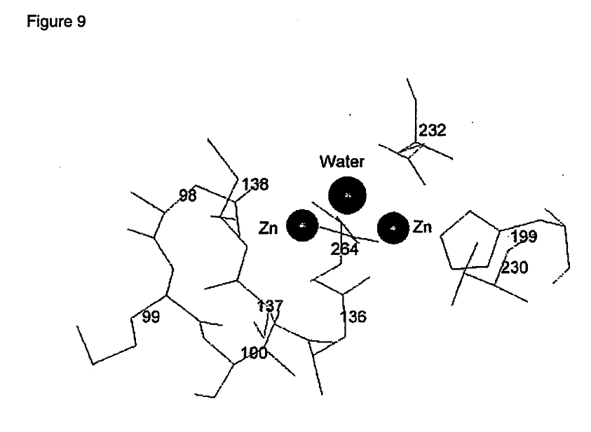

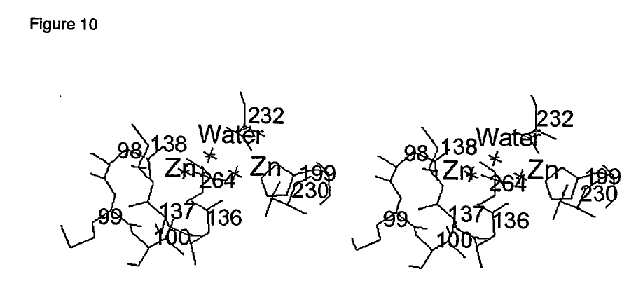

One method that may be employed for this purpose is molecular replacement. In this method, an unknown crystal structure, whether it is another crystal form of MetAP, a MetAP mutant, a MetAP co-complex, a MetAP from a different bacterial species, or a crystal of some other protein with significant amino acid sequence homology to any domain of MetAP, may be determined using MetAP structure coordinates of this invention, such as those provided in FIGS. 1-10 and Tables I-X. This method may provide an accurate structural form for an unknown crystal.

Thus, MetAP structures provided herein permits screening of known molecules and/or the designing of new molecules that bind to a structure, particularly at a binding pocket or active site, via use of computerized evaluation systems. For example, computer modeling systems are available in which a sequence of a MetAP, and a MetAP structure (i.e., atomic coordinates, bond distances between atoms in the active site region, etc. as provided, for example, by Tables I-X herein) may be input. Thus, a machine readable medium may be encoded with data representing coordinates of Tables I-X. The computer may then generate structural details of a site into which a test compound may bind, thereby enabling determination of a complementary structural details of this test compound.

More particularly, design of compounds that bind to or inhibit MetAP according to this invention generally involves consideration of two factors. First, a compound must be capable of physically and structurally associating with MetAP. Non-covalent molecular interactions important in an association of MetAP with its substrate include hydrogen bonding, van der Waals, and hydrophobic interactions.

Second, a compound must be able to assume a conformation that allows it to associate with MetAP. Although certain portions of a compound may not directly participate in this association with MetAP, those portions may still influence an overall conformation of a molecule. This, in turn, may have a significant impact on potency. Such conformational requirements include an overall three-dimensional structure and orientation of the chemical entity or compound in relation to all or a portion of a binding site, e.g., binding pocket, active site, or substrate binding sites of MetAP, or a spacing between functional groups of a compound comprising several chemical entities that directly interact with MetAP.

Another approach made possible by this invention is to screen computationally small molecule databases for chemical entities or compounds that can bind in whole, or in part, to a MetAP enzyme. Details on this process and the results it can provide are now documented in the art. For a description of this type of technology please refer to PCT application WO 97/16177 published 9 May 1997; the techniques described there for computer modeling are incorporated herein by reference.

Once identified by modeling techniques, a MetAP inhibitor may be tested for bio-activity using standard techniques. For example, a structure of the invention may be used in enzymatic activity assays to determine an inhibitory activity of compounds or binding assays using conventional formats to screen inhibitors. One particularly suitable assay format includes a enzyme-linked immunosorbent assay (herein “ELISA”). Other assay formats may be used; these assay formats are not a limitation on the present invention.

A potential inhibitory or binding effect of a chemical compound on MetAP may be analyzed prior to its actual synthesis and testing by the use of computer modelling techniques. If a theoretical structure of a given compound suggests insufficient interaction and association between it and MetAP, synthesis and testing of the compound is obviated. However, if computer modelling indicates a strong interaction, a molecule may then be synthesized and tested for its ability to bind to MetAP and inhibit using a suitable assay. In this manner, synthesis of inoperative compounds may be avoided.

An inhibitory or other binding compound of MetAP may be computationally evaluated and designed by means of a series of steps in that chemical entities or fragments are screened and selected for their ability to associate with an individual binding pockets or other areas of MetAP.

One skilled in the art may use one of several methods to screen chemical entities or fragments for their ability to associate with MetAP and more particularly with individual binding pockets of a MetAP active site or accessory binding site. This process may begin by visual inspection of, for example, an active site on a computer screen based on MetAP coordinates in Tables I-X. Selected fragments or chemical entities may then be positioned in a variety of orientations, or docked, within an binding pocket or active site of MetAP. Docking may be accomplished using software such as Quanta and Sybyl, followed by energy minimization and molecular dynamics with standard molecular mechanics forcefields, such as CHARMM and AMBER.

Specialized computer programs may also assist in the process of selecting fragments or chemical entities. These include, but are not limited to:

1. GRID (P. J. Goodford, “A Computational Procedure for Determining Energetically Favorable Binding Sites on Biologically Important Macromolecules”, J. Med. Chem., 28:849-857 (1985)). GRID is available from Oxford University, Oxford, UK.

2. MCSS (A. Miranker and M. Karplus, “Functionality Maps of Binding Sites: A Multiple Copy Simultaneous Search Method”, Proteins: Structure, Function and Genetics, 11:29-34 (1991)). MCSS is available from Molecular Simulations, Burlington, Mass.

3. AUTODOCK (D. S. Goodsell and A. J. Olsen, “Automated Docking of Substrates to Proteins by Simulated Annealing”, Proteins: Structure, Function, and Genetics, 8:195-202 (1990)). AUTODOCK is available from Scripps Research Institute, La Jolla, Calif.

4. DOCK (I. D. Kuntz et al., “A Geometric Approach to Macromolecule-Ligand Interactions”, J. Mol. Biol., 161:269-288 (1982)). DOCK is available from University of California, San Francisco, Calif.

In addition, other commercially available computer databases for small molecular compounds includes Cambridge Structural Database, Fine Chemical Database, and CONCORD, for a review see Rusinko, A., Chem. Des. Auto. News 8, 44-47 (1993).

Once suitable chemical entities or fragments have been selected, they may be assembled into a single compound or inhibitor. Assembly may be proceed by visual inspection of a relationship of the fragments to each other on a three-dimensional image displayed on a computer screen in relation to structural coordinates of MetAP. This may be followed by manual model building using software such as Quanta or Sybyl.

Useful programs to aid one of skill in the art in connecting the individual chemical entities or fragments include, but are not limited to:

1. CAVEAT (P. A. Bartlett et al, “CAVEAT: A Program to Facilitate the Structure-Derived Design of Biologically Active Molecules”, in Molecular Recognition in Chemical and Biological Problems”, Special Pub., Royal Chem. Soc. 78, pp. 182-196 (1989)]. CAVEAT is available from the University of California, Berkeley, Calif.

2. 3D Database systems such as MACCS-3D (MDL Information Systems, San Leandro, Calif.). This area is reviewed in Y. C. Martin, “3D Database Searching in Drug Design,” J. Med. Chem., 35:2145-2154 (1992).

3. HOOK (available from Molecular Simulations, Burlington, Mass.).

Instead of proceeding to build a MetAP modulator in a step-wise fashion a fragment or chemical entity at a time as described above, inhibitory, modulatory or other MetAP binding compounds may be designed as a whole or “de novo” using an empty active site or optionally including some portion(s) of a known ligand(s). These methods include, but are not limited to:

1. LUDI (H. J. Bohm, “The Computer Program LUDI: A New Method for the De Novo Design of Enzyme Inhibitors”, J. Comp. Aid. Molec. Design, 6:61-78 (1992)). LUDI is available from Biosym Technologies, San Diego, Calif.

2. LEGEND (Y. Nishibata and A. Itai, Tetrahedron, 47:8985 (1991)). LEGEND is available from Molecular Simulations, Burlington, Mass.

3. LeapFrog (available from Tripos Associates, St. Louis, Mo.).

Other molecular modelling techniques may also be employed in accordance with this invention. See, e.g., N. C. Cohen et al, “Molecular Modeling Software and Methods for Medicinal Chemistry”, J. Med. Chem., 33:883-894 (1990). See also, M. A. Navia and M. A. Murcko, “The Use of Structural Information in Drug Design”, Current Opinions in Structural Biology, 2:202-210 (1992). For example, where the structures of test compounds are known, a model of a test compound may be superimposed over a model of a structure of the invention. Numerous methods and techniques are known in the art for performing this step, any of which may be used. See, e.g., P. S. Farmer, Drug Design, Ariens, E. J., ed., Vol. 10, pp 119-143 (Academic Press, New York, 1980); U.S. Pat. No. 5,331,573; U.S. Pat. No. 5,500,807; C. Verlinde, Structure, 2:577-587 (1994); and 1. D. Kuntz, Science, 257:1078-1082 (1992). Model building techniques and computer evaluation systems described herein are not a limitation on the present invention.

Thus, using these computer evaluation systems, a large number of compounds may be examined. Moreover, the need for synthesis of many compounds may be eliminated.

In another aspect, a bacterial methionine aminopeptidase structure of the invention may permit design and identification of synthetic compounds and/or other molecules that may be characterized by a conformation of a bacterial methionine aminopeptidase of the invention. Using known computer systems, coordinates of the bacterial methionine aminopeptidase structures of the invention may be provided in machine readable form, test compounds designed and/or screened and their conformations superimposed on a structure of the methionine aminopeptidases of the invention. Subsequently, suitable candidates identified as above may be screened for a desired methionine aminopeptidase inhibitory activity, stability, and the like.

Once identified and screened for activity, these inhibitors may be used therapeutically or prophylactically to block methionine aminopeptidase activity, and thus, overcome bacterial resistance to antibiotics, for example, of the beta-lactam class, e.g. imipenem, penicillins, cephalosporins, etc. by using a different mechanism of attacking bacteria in diseases produced by bacterial infection.

All publications, including, but not limited to, patents and patent applications cited in this specification, are herein incorporated by reference as if each individual publication were specifically and individually indicated to be incorporated by reference herein as though fully set forth.

Tables

Table I provides three dimensional protein coordinates of the S. aureus methionine aminopeptidase crystalline structure of the invention.

Table II provides three dimensional coordinates for a S. aureus methionine aminopeptidase complex with a specific inhibitor of the present invention, 5-(3-Iodo-phenyl)-1-H-[1,2,3]triazole.

Table III provides three dimensional coordinates for a S. aureus methionine aminopeptidase complex with a specific inhibitor of the present invention, 5-benzofuran-2-yl-1-H-[1,2,3]triazole.

Table IV provides distances between interresidue atoms that are within 5 Ångstroms apart in an active site of S. aureus methionine aminopeptidase for inhibitor complexes of a specific inhibitor of the present invention, 5-(3-Iodo-phenyl)-1-H-[1,2,3]triazole.

Table V provides distances between interresidue atoms that are within 5 Ångstroms apart in an active site of S. aureus methionine aminopeptidase for inhibitor complexes of a specific inhibitor of the present invention, 5-benzofuran-2-yl-1-H-[1,2,3]triazole.

Table VI provides angles between interresidue atoms that are within 5 ÅAngstroms apart in an active site of S. aureus methionine aminopeptidase for inhibitor complexes of a specific inhibitor of the present invention, 5-(3-Iodo-phenyl)-1-H-[1,2,3]triazole.

Table VII provides angles between interresidue atoms that are within 5 Ångstroms apart in an active site of S. aureus methionine aminopeptidase for inhibitor complexes of a specific inhibitor of the present invention, 5-benzofuran-2-yl-1-H-[1,2,3]triazole.

Table VIII provides three dimensional protein coordinates of an S. pneumoniae methionine aminopeptidase crystalline structure of the invention.

Table IX provides distances between interresidue atoms that are within 5 Ångstroms apart in an active site of S. pneumoniae methionine aminopeptidase.

Table X provides angles between interresidue atoms that are within 5 Ångstroms apart in an active site of S. pneumoniae.

Table Legend:

1. Under the heading ATOM appears a “atom number” (e.g. 1, 2, 3, 4 . . . etc) and the “atom name” (e.g. CA, CB, N, . . . etc) such that to each “atom name” in a coordinate list corresponds an “atom number”.
2. Under the heading RESIDUE appears a three-letter “residue name” (e.g. THR, ASP, etc), a “chain identifier” represented by a capital letter (e.g. A, B, C D, etc) and a “residue number”, such that to each residue (or amino acid) in an amino acid sequence of a particular protein in a structure corresponds a name that identifies it, a number according to its position along an amino acid sequence, and a chain name. A chain name identifies a particular molecule in a crystal structure. For instance, if there is more than one molecule that forms a unit that is repeated throughout a crystal lattice, then each unit is identified as molecule A, or molecule B, or molecule C, etc.
3. Under the headings X, Y, or Z appear Cartesian coordinates of the atoms in a structure.
4. Under the heading B appears the “B-factor” or “temperature factor” that may adopt, in principle, any value. It is meant to represent an atomic displacement around that position.

### EXAMPLES

The invention will now be described by reference to the following examples which are merely illustrative and are not to be construed as a limitation of the scope of the present invention. In the Examples, proton NMR spectra were performed upon a Bruker 400 MHz NMR spectrometer, unless otherwise indicated.

### Example 1

### Preparation of 3-(1H-1,2,3-triazol-4-yl)-phenol

To a stirring solution of 3-ethynylphenol (0.55 g, 4.0 mmol) in 4 ml of toluene under an inert atmosphere was added trimethylsilylazide (1 ml, 8 mmol). The resulting solution was heated to reflux for 3 days. To this mixture was added water (1 ml) and after evaporation, the resulting residue was purified by preparative HPLC to afford the title compound as a white solid (0.12 g, 18%). ¹H-NMR (400 MHz, CD₃OD): δ 8.09 (s, 1H), 7.27 (m, 3H), 6.81 (m, 1H). MS (ESI) 162.2 (M+H)⁺. (This procedure was adapted from Tanaka, Y.; Velen, S. R.; Miller, S. I. Tetrahedron, 1973, 29, 3271.)

### Example 2

### Preparation of 4-(3-iodophenyl)-1H-1,2,3-triazole

Following the procedure of Example 1, except substituting 1-ethynyl-3-iodobenzene for 3-ethynylphenol, the title compound was prepared as a white solid (20%). ¹H-NMR (400 MHz, CDCl₃): δ 8.21 (s, 1H), 7.98 (s, 1H), 7.81 (d, J=7.8 Hz, 1H), 7.73 (d, J=8.1 Hz, 1H), 7.21 (t, J=7.8 Hz, 1H). MS (ESI) 272.0 (M+H)⁺.

### Example 3

### Preparation of 4-(2-fluorophenyl)-1H-1,2,3-triazole

Following the procedure of Example 1, except substituting 1-ethynyl-2-fluorobenzene for 3-ethynylphenol, the title compound was prepared as a white solid (21%). ¹H-NMR (400 MHz, CDCl₃): δ 11.54 (brs, 1H), 8.19 (s, 1H), 8.11 (t, J=7.5 Hz, 1H), 7.18-7.40 (m, 3H). MS (ESI) 164.2 (M+H)⁺.

### Example 4

### Preparation of 4-(4-n-butylphenyl)-1H-1,2,3-triazole

Following the procedure of Example 1, except substituting 1-ethynyl-4-n-butylbenzene for 3-ethynylphenol, the title compound was prepared as a white solid (16%). ¹H-NMR (400 MHz, CD₃OD): δ 8.11 (s, 1H), 7.74 (d, J=7.8 Hz, 2H), 7.28 (d, J=8.0 Hz, 2H), 2.67 (t, J=7.6 Hz, 2H), 1.61-1.69 (m, 2H), 1.37-1.43 (m, 2H), 0.97 (t, J=7.3 Hz, 3H). MS (ESI) 202.2 (M+H)⁺.

### Example 5

### Preparation of 4-(2-chlorophenyl)-1H-1,2,3-triazole

Following the procedure of Example 1, except substituting 1-chloro-2-ethynylbenzene for 3-ethynylphenol, the title compound was prepared as a white solid (35%). ¹H-NMR (400 MHz, CD₃OD): δ 8.29 (s, 1H), 7.90 (d, J=7.0 Hz, 1H), 7.53-7.56 (m, 1H), 737-7.44 (m, 2H). MS (ESI) 180.0 (M+H)⁺.

### Example 6

### Preparation of N-(3-[1H-1,2,3-triazol-4-yl]phenyl)benzamide

Following the procedure of Example 1, except substituting N-(3-ethynylphenyl)benzamide for 3-ethynylphenol, the title compound was prepared as a white solid (12%). ¹H-NMR (400 MHz, CD₃OD): δ 8.18-8.20 (m, 2H), 7.93-8.00 (m, 2H), 7.45-7.76 (m, 6H). MS (ESI) 265.2 (M+H)⁺.

### Example 7

### Preparation of 3-(1H-1,2,3-triazol-4-yl)-phenylamine

Following the procedure of Example 1, except substituting 3-ethynyl-phenylamine for 3-ethynylphenol, the title compound was prepared as a tan solid (19%). ¹H-NMR (400 MHz, CD₃OD): δ 8.05 (s, 1H), 7.12-7.20 (m, 3H), 6.73-6.75 (m, 1H). MS (ESI) 161.2 (M+H)⁺.

### Example 8

### Preparation of N-(3-[1H-1,2,3-triazol-4-yl]phenyl)acetamide

Following the procedure of Example 1, except substituting N-(3-ethynylphenyl)acetamide for 3-ethynylphenol, the title compound was prepared as a tan solid (49%). ¹H-NMR (400 MHz, DMSO-d₆): δ 10.04 (s, 1H), 8.11-8.50 (m, 2H), 7.35-7.58 (m, 3H), 2.06 (s, 3H). MS (ESI) 203.2 (M+H)⁺.

### Example 9

### Preparation of 4-(4-trifluoromethylphenyl)-1H-1,2,3-triazole

Following the procedure of Example 1, except substituting 1-ethynyl-4-trifluoromethylphenyl for 3-ethynylphenol, the title compound was prepared as a white solid (50%). ¹H-NMR (400 MHz, CD₃OD): δ 8.30 (s, 1H), 8.06 (d, J=8.2 Hz, 2H), 7.76 (d, J=8.2 Hz, 2H). MS (ESI) 214.2 (M+H)⁺.

### Example 10

### Preparation of 4-(3-trifluoromethylphenyl)-1H-1,2,3-triazole

Following the procedure of Example 1, except substituting 1-ethynyl-3-trifluoromethylphenyl for 3-ethynylphenol, the title compound was prepared as a white solid (16%). ¹H-NMR (400 MHz, CD₃OD): δ8.32, (s, 1H), 8.10-8.18 (m, 2H), 7.64-7.68 (m, 1H). MS (ESI) 214.2 (M+H)⁺.

### Example 11

### Preparation of 4-(4-n-propylphenyl)-1H-1,2,3-triazole

Following the procedure of Example 1, except substituting 1-ethynyl-4-n-propylbenzene for 3-ethynylphenol, the title compound was prepared as a white solid (26%). ¹H-NMR (400 MHz, CD₃OD): δ 8.11 (s, 1H), 7.74 (d, J=7.5 Hz, 2H), 7.28 (d, J=8.0 Hz, 2H), 2.64 (t, J=7.6 Hz, 2H), 1.64-1.73 (m, 2H), 0.97 (t, J=7.3 Hz, 3H). MS (ESI) 188.2 (M+H)⁺.

### Example 12

### Preparation of 4-(4-methoxyphenyl)-1H-1,2,3-triazole

Following the procedure of Example 1, except substituting 1-ethynyl-4-methoxybenzene for 3-ethynylphenol, the title compound was prepared as a white solid (34%). ¹H-NMR (400 MHz, CDCl₃): δ 7.92 (s, 1H), 7.76 (d, J=8.8 Hz, 2H), 7.01 (d, J=8.8 Hz, 2H), 3.88 (s, 3H). MS (ESI) 176.2 (M+H)⁺.

### Example 13

### Preparation of 4-(3-methylphenyl)-1H-1,2,3-triazole

Following the procedure of Example 1, except substituting 3-ethynyltoluene for 3-ethynylphenol, the title compound was prepared as a white solid (23%). ¹H-NMR (400 MHz, CD₃OD): δ 8.14 (s, 1H), 7.67 (s, 1H), 7.62 (d, J=7.7 Hz, 1H), 7.34 (t, J=7.6 Hz, 1H), 7.20 (d, J=7.6 Hz, 1H), 2.41 (s, 3H). MS (ESI) 160.2 (M+H)⁺.

### Example 14

### Preparation of 2-(1H-1,2,3-triazolyl)-pyridine

Following the procedure of Example 1, except substituting 2-ethynylpyridine for 3-ethynylphenol, the title compound was prepared as a white solid (16%). ¹H-NMR (400 MHz, CD₃OD): δ 8.60-8.61 (m, 1H), 8.32 (s, 1H), 8.06 (d, J=8.0 Hz, 1H), 7.90-7.95 (m, 1H), 7.38-7.41 (m, 1H). MS (ESI) 147.2 (M+H)⁺.

### Example 15

### Preparation of 4-(4-chlorophenyl)-1H-1,2,3-triazole

Following the procedure of Example 1, except substituting 1-chloro-4-ethynylbenzene for 3-ethynylphenol, the title compound was prepared as a white solid (35%). ¹H-NMR (400 MHz, CD₃OD): δ 8.18 (s, 1H), 7.85 (d, J=8.6 Hz, 2H), 7.47 (d, J=8.7 Hz, 2H). MS (ESI) 180.0 (M+H)⁺.

### Example 16

### Preparation of 4-(4-ethylphenyl)-1H-1,2,3-triazole

Following the procedure of Example 1, except substituting 1-ethyl-4-ethynylbenzene for 3-ethynylphenol, the title compound was prepared as a white solid (11%). ¹H-NMR (400 MHz, CD₃OD): δ 8.11 (s, 1H), 7.74 (d, J=8.2 Hz, 2H), 7.30 (d, J=8.2 Hz, 2H), 2.69 (q, J=7.6, 2H), 1.27 (t, J=7.6 Hz, 3H). MS (ESI) 174.2 (M+H)⁺.

### Example 17

### Preparation of 4-(1H-1,2,3-triazol-4-yl)-phenylamine

Following the procedure of Example 1, except substituting 4-ethynylphenylamine for 3-ethynylphenol, the title compound was prepared as an orange solid (9%). ¹H-NMR (400 MHz, CD₃OD): δ 7.94 (s, 1H), 7.54 (d, J=8.6 Hz, 2H), 6.78 (d, J=8.6 Hz, 2H). MS (ESI) 161.2 (M+H)⁺.

### Example 18

### Preparation of 4-(4-methylphenyl)-1H-1,2,3-triazole

Following the procedure of Example 1, except substituting 4-ethynyltoluene for 3-ethynylphenol, the title compound was prepared as a white solid (14%). ¹H-NMR (400 MHz, CDCl₃): δ 7.96 (s, 1H), 7.73 (d, J=8.0 Hz, 2H), 7.28-7.30 (m, 2H), 1.57 (s, 3H). MS (ESI) 160.2 (M+H)⁺.

### Example 19

### Preparation of 2-(1H-1,2,3-triazol-4-yl)-5-methylpyridine

Following the procedure of Example 1, except substituting 2-ethynyl-5-methylpyridine (Sakamoto, T.; Nagata, H.; Kondo, Y.; Sato, K.; Yamanaka, H. Chem. Pharm. Bull. 1984, 32, 4866) for 3-ethynylphenol, the title compound was prepared as a white solid (28%). ¹H-NMR (400 MHz, CD₃OD): δ 8.45 (s, 1H), 8.27 (s, 1H), 7.95 (d, J=8.1 Hz, 1H), 7.76 (d, J=8.1 Hz, 1H), 2.41 (s, 3H). MS (ESI) 161.2 (M+H)⁺.

### Example 20

### Preparation of 2-(1H-1,2,3-triazol-4-yl)-4-methyl-pyridine

Following the procedure of Example 1, except substituting 2-ethynyl-4-methylpyridine (Sakamoto, T.; Nagata, H.; Kondo, Y.; Sato, K.; Yamanaka, H. Chem. Pharm. Bull. 1984, 32, 4866) for 3-ethynylphenol, the title compound was prepared as a white solid (54%). ¹H-NMR (400 MHz, CD₃OD): δ 8.45 (d, J=5.1 Hz, 1H), 8.29 (s, 1H), 7.91 (s, 1H), 7.23 (d, J=5.1 Hz, 1H), 2.46 (s, 3H). MS (ESI) 161.2 (M+H)⁺.

### Example 21

### Preparation of 1-(1H-1,2,3-triazol-4-yl)cyclohexanol

Following the procedure of Example 1, except substituting 1-ethynylcyclohexanol for 3-ethynylphenol, the title compound was prepared as a white solid (10%). ¹H-NMR (400 MHz, CD₃OD): δ7.70 (s, 1H), 1.39-1.99 (m, 10H). MS (ESI) 168.2 (M+H)⁺.

### Example 22

### Preparation of 4-(thiophen-2-yl)-1H-1,2,3-triazole

### a) 2-ethynylthiophene

To a stirring solution of 2-thiophenecarboxaldehyde (0.33 g, 3.0 mmol) in dry methanol (30 ml) was added potassium carbonate (0.87 g, 6.3 mmol) and 1-diazo-2-oxopropylphosphonate (0.78 g, 4.1 mmol, Calant, P.; D'Haenens, L.; Vandewalle, M. Synth. Commun. 1984, 14, 155). After 4 h of stirring at room temperature, aqueous sodium bicarbonate (5%, 50 ml) and hexanes (50 ml) were added. The organic layer was collected, dried (MgSO₄) and filtered through a short silica plug. Evaporation yielded the title compound as a clear oil. (This procedure was adapted from Muller, S.; Liepold, B.; Roth, G. J.; Bestmann, H. J. Synlett 1996, 521.)

### b) 4-(thiophen-2-yl)-1H-1,2,3-triazole

Following the procedure of Example 1, except substituting 2-ethynylthiophene for 3-ethynylphenol, the title compound was prepared as a white solid (2 steps, 7%). ¹H-NMR (400 MHz, CD₃OD): δ 8.05 (s, 1H), 7.43-7.47 (m, 2H), 7.10-7.13 (m, 1H). MS (ESI) 152.2 (M+H)⁺.

### Example 23

### Preparation of 4-(thiophenyl)-1H-1,2,3-triazole

Following the procedure of Example 22, except substituting 3-thiophenecarboxaldehyde for 2-thiophenecarboxaldehyde in step a, the title compound was prepared as a white solid (2 steps, 8%). ¹H-NMR (400 MHz, CD₃OD): δ 8.07 (s, 1H), 7.79 (s, 1H), 7.53 (s, 2H). MS (ESI) 152.2 (M+H)⁺.

### Example 24

### Preparation of 4-(2-methylphenyl)-1H-1,2,3-triazole

Following the procedure of Example 22, except substituting o-tolualdehyde for 2-thiophenecarboxaldehyde in step a, the title compound was prepared as a white solid (2 steps, 3%). ¹H-NMR (400 MHz, CD₃OD): δ 7.97 (s, 1H), 7.55-7.58 (m, 1H), 7.26-7.33 (m, 3H), 2.44 (s, 3H). MS (ESI) 160.2 (M+H)⁺.

### Example 25

### Preparation of 4-(1,3-dimethylphenyl)-1H-1,2,3-triazole

Following the procedure of Example 22, except substituting 2,4-dimethylbenzaldehyde for 2-thiophenecarboxaldehyde in step a, the title compound was prepared as a white solid (2 steps, 3%). ¹H-NMR (400 MHz, CD₃OD): δ 7.92 (s, 1H), 7.44 (d, J=7.8 Hz, 1H), 7.14 (s, 1H), 7.10 (d, J=7.3 Hz, 1H), 2.40 (s, 3H), 2.36 (s, 3H). MS (ESI) 174.2 (M+H)⁺.

### Example 26

### Preparation of 4-(4-bromophenyl)-1H-1,2,3-triazole

Following the procedure of Example 22, except substituting 4-bromobenzaldehyde for 2-thiophenecarboxaldehyde in step a, the title compound was prepared as a white solid (2 steps, 7%). ¹H-NMR (400 MHz, CD₃OD): δ 8.19 (s, 1H), 7.77 (d, J=8.6 Hz, 2H), 7.61 (d, J=8.6, 2H). MS (ESI) 224.0 (M+H)⁺.

### Example 27

### Preparation of 4-(1,3-dichlorophenyl)-1H-1,2,3-triazole

Following the procedure of Example 22, except substituting 2,4-dichlorobenzaldehyde for 2-thiophenecarboxaldehyde in step a, the title compound was prepared as a white solid (2 steps, 6%). ¹H-NMR (400 MHz, CD₃OD): δ (s, 1H), 7.91-7.94 (m, 1H), 7.61-7.62 (m, 1H) 7.44-7.48 (m, 1H). MS (ESI) 214.0 (M+H)⁺.

### Example 28

### Preparation of 4-(1-biphenyl-2-yl)-1H-1,2,3-triazole

Following the procedure of Example 22, except substituting 2-biphenylcarboxaldehyde for 2-thiophenecarboxaldehyde in step a, the title compound was prepared as a clear oil (2 steps, 27%). ¹H-NMR (400 MHz, CD₃OD): δ 7.80 (s, 1H), 7.47-7.49 (m, 2H), 7.36-7.40 (m, 4H), 7.20-7.22 (m, 2H), 6.88 (s, 1H). MS (ESI) 222.2 (M+H)⁺.

### Example 29

### Preparation of 4-(2-benzyloxy-phenyl)-1H-1,2,3-triazole

Following the procedure of Example 22, except substituting 2-benzyloxybenzaldehyde for 2-thiophenecarboxaldehyde in step a, the title compound was prepared as a white solid (2 steps, 25%). ¹H-NMR (400 MHz, CD₃OD): δ 8.09 (s, 1H), 8.00 (d, J=7.7 Hz, 1H), 7.33-7.43 (m, 6H), 7.20 (d, J=8.3 Hz, 1H), 7.07 (t, J=7.5, 1H), 5.25 (s, 2H). MS (ESI) 252.2 (M+H)⁺.

### Example 30

### Preparation of 2-(1H-1,2,3-triazol-4-yl)-6-methylpyridine

Following the procedure of Example 22, except substituting 6-methyl-2-pyridine carboxaldehyde for 2-thiophenecarboxaldehyde in step a, the title compound was prepared as a clear oil (2 steps, 39%). ¹H-NMR (400 MHz, CD₃OD): δ 8.33 (s, 1H), 7.77-7.85 (m, 2H), 7.26 (d, J=7.4 Hz, 1H), 2.60 (s, 3H). MS (ESI) 161.2 (M+H)⁺.

### Example 31

### Preparation of 3-(1H-1,2,3-triazol-4-yl)-pyridine

Following the procedure of Example 22, except substituting 3-pyridine carboxaldehyde for 2-thiophenecarboxaldehyde in step a, the title compound was prepared as a white solid (2 steps, 25%). %). ¹H-NMR (400 MHz, CD₃OD): δ 9.06 (s, 1H), 8.54 (d, J=3.4 Hz, 1H), 8.31-8.33 (m, 2H), 7.54 (m, 1H). MS (ESI) 147.2 (M+H)⁺.

### Example 32

### Preparation of 4-(1H-1,2,3-triazol-4-yl)-pyridine

Following the procedure of Example 22, except substituting 4-pyridine carboxaldehyde for 2-thiophenecarboxaldehyde in step a, the title compound was prepared as a white solid (2 steps, 15%). ¹H-NMR (400 MHz, CD₃OD): δ 8.61-8.62 (m, 2H), 8.42 (s, 1H), 7.91-7.93 (m, 2H). MS (ESI) 147.2 (M+H)⁺.

### Example 33

### Preparation of 4-(2-methoxyphenyl)-1H-1,2,3-triazole

Following the procedure of Example 22, except substituting o-anisaldehyde for 2-thiophenecarboxaldehyde in step a, the title compound was prepared as a white solid (2 steps, 6%). ¹H-NMR (400 MHz, CD₃OD): δ 8.19 (s, 1H), 7.95 (d, J=6.8 Hz, 1H), 7.35-7.40 (m, 1H), 7.14 (d, J=8.3 z, 1H), 7.04-7.08 (m, 1H), 4.90 (s, 3H). MS (ESI) 176.2 (M+H)⁺.

### Example 34

### Preparation of 4-(2-bromophenyl)-1H-1,2,3-triazole

Following the procedure of Example 22, except substituting 2-bromobenzaldehyde for 2-thiophenecarboxaldehyde in step a, the title compound was prepared as a white solid (2 steps, 15%). ¹H-NMR (400 MHz, CD₃OD): δ 8.27 (s, 1H), 7.73-7.79 (m, 2H), 7.45-7.79 (m, 1H), 7.30-7.34 (m, 1H). MS (ESI) 224.0 (M+H)⁺.

### Example 35

### Preparation of 4-benzo[1,3]dioxol-5-yl-1H-1,2,3-triazole

Following the procedure of Example 22, except substituting piperonal for 2-thiophenecarboxaldehyde in step a, the title compound was prepared as a white solid (2 steps, 10%). ¹H-NMR (400 MHz, CD₃OD): δ 8.05 (s, 1H), 7.32-7.34 (m, 2H), 6.89-6.91 (m, 1H), 6.00 (s, 2H). MS (ESI) 190.2 (M+H)⁺.

### Example 36

### Preparation of 2-(1H-1,2,3-triazol-4-yl)-benzofuran

Following the procedure of Example 22, except substituting benzofuran-2-carboxaldehyde for 2-thiophenecarboxaldehyde in step a, the title compound was prepared as a white solid (2 steps, 25%). ¹H-NMR (400 MHz, CD₃OD): δ 8.25 (s, 1H), 7.65 (d, J=7.6 Hz, 1H), 7.56 (d, J=8.0 Hz, 1H), 7.23-7.36 (m, 3H). MS (ESI) 186.0 (M+H)⁺.

### Example 37

### Preparation of 4-benzo[1,3]dioxol-4-yl-1H-1,2,3-triazole

### a) 4-ethynyl-benzo[1,3]dioxole

Following the procedure of Example 22, except substituting benzo[1,3]dioxole-4-carbaldehyde for 2-thiophenecarboxaldehyde in step a, the title compound was obtained as an oil (98%). ¹H-NMR (400 MHz, CDCl₃): δ 6.94-6.96 (m, 1H), 6.80-6.85 (m, 2H), 6.05 (s, 2H), 3.30 (s, 1H).

### b) 4-benzo[1,3]dioxol-4-yl-1H-1,2,3-triazole

Following the procedure of Example 1, except substituting 4-ethynyl-benzo[1,3]dioxole for 3-ethynylphenol, the title compound was prepared as a white solid (24%). ¹H-NMR (400 MHz, CD₃OD): δ 8.13 (s, 1H), 7.45 (d, J=8.0 Hz, 1H), 6.94-6.97 (m, 1H), 6.81-6.83 (m, 1H), 6.08 (s, 2H). MS (ESI) 190.2 (M+H)⁺.

### Example 38

### Preparation of 4-(2-[4-chloro-phenylsulfanyl]-phenyl)-1H-1,2,3-triazole

### a) 1-(4-chloro-phenylsulfanyl)-2-ethynylbenzene

Following the procedure of Example 22, except substituting 2-(4-chlorophenylthio)benzaldehyde for 2-thiophenecarboxaldehyde in step a, the title compound was obtained as an oil (91%). ¹H-NMR (400 MHz, CDCl₃): δ 7.53-7.55 (m, 1H), 7.17-7.40 (m, 6H), 7.03-7.05 (m, 1H), 3.43 (s, 1H).

### b) 4-(2-[4-chloro-phenylsulfanyl]-phenyl)-1H-1,2,3-triazole

Following the procedure of Example 1, except substituting 1-(4-chloro-phenylsulfanyl)-2-ethynylbenzene for 3-ethynylphenol, the title compound was prepared as a white solid (21%). ¹H-NMR (400 MHz, CD₃OD): δ 8.10 (s, 1H), 7.77-7.79 (m, 1H), 7.39-7.46 (m, 3H), 7.28-7.30 (m, 2H), 7.15-7.17 (m, 2H). MS (ESI) 288.2 (M+H)⁺.

### Example 39

### Preparation of (3-phenyl-propyl)-(3-[1H-1,2,3-triazol-4-yl]phenyl)amine

### a) (3-phenyl-propyl)-(3-ethynylphenyl)amine

To a stirring solution of 3-ethynylphenylamine (0.59 g, 5.0 mmol) and 3-phenylpropionaldehyde (0.66 g, 5.0 mmol) in 1,2-dichloroethane (15 ml) was added acetic acid (0.29 ml, 5.0 mmol) and sodium triacetoxyborohydride (1.6 g, 7.5 mmol). After stirring at room temperature for 72 h, aqueous sodium bicarbonate (saturated) and diethyl ether were added. The organic layer was washed with additional sodium bicarbonate, dried (MgSO₄) and evaporated. Purification via silica gel chromatography gave the title compound as a clear oil (42%). MS (ESI) 236.2 (M+H)⁺.

### b) (3-phenyl-propyl)-(3-[1H-1,2,3-triazol-4-yl]phenyl)amine

Following the procedure of Example 1, except substituting (3-phenyl-propyl)-(3-ethynylphenyl)amine for 3-ethynylphenol, the title compound was prepared as a clear oil (16%). ¹H-NMR (400 MHz, CD₃OD): δ 8.03 (s, 1H), 7.6.62-7.30 (m, 9H), 3.17 (t, J=7.0 Hz, 2H), 2.77 (t, J=7.4 Hz, 2H), 1.97 (t, J=7.7 Hz, 2H). MS (ESI) 279.4 (M+H)⁺.

### Example 40

### Preparation of phenethyl-(3-[1H-1,2,3-triazol-4-yl]phenyl)amine

### a) phenethyl-(3-ethynylphenyl)-amine

Following the procedure of Example 39, except substituting phenylacetaldehyde for 3-phenylpropionaldehyde in step a, the title compound was prepared as a clear oil (47%). MS (ESI) 222.2 (M+H)⁺.

### b) phenethyl-(3-[1H-1,2,3-triazol-4-yl]phenyl)amine

Following the procedure of Example 1, except substituting phenethyl-(3-ethynylphenyl)-amine for 3-ethynylphenol, the title compound was prepared as a clear oil (19%). ¹H-NMR (400 MHz, CD₃OD): δ 8.07 (s, 1H), 7.06-7.31 (m, 8H), 6.67 (d, J=8.1 Hz, 1H), 3.41 (t, J=7.2 Hz, 2H), 2.94 (t, J=7.1 Hz, 2H). MS (ESI) 265.2 (M+H)⁺.

### Example 41

### Preparation of furan-2-ylmethyl-(3-[1H-1,2,3-triazol-4-yl]phenyl)amine

### a) furan-2-ylmethyl-(3-ethynylphenyl)-amine

Following the procedure of Example 39, except substituting furfural for 3-phenylpropionaldehyde in step a, the title compound was prepared as a clear oil (75%). MS (ESI) 198.2 (M+H)⁺.

### b) furan-2-ylmethyl-(3-[1H-1,2,3-triazol-4-yl]phenyl)amine

Following the procedure of Example 1, except substituting furan-2-ylmethyl-(3-ethynylphenyl)-amine for 3-ethynylphenol, the title compound was prepared as a white solid (18%). ¹H-NMR (400 MHz, CD₃OD): δ 8.06 (s, 1H), 7.43 (d, J=1.0 Hz, 1H), 7.09-7.22 (m, 3H), 6.72 (d, J=8.1 Hz, 1H), 6.34-6.35 (m, 1H), 6.28 (d, J=3.2 Hz, 1H), 4.36 (s, 2H). MS (ESI) 241.2 (M+H)⁺.

### Example 42

### Preparation of furan-3-ylmethyl-(3-[1H-1,2,3-triazol-4-yl]phenyl)amine

### a) furan-3-ylmethyl-(3-ethynylphenyl)-amine

Following the procedure of Example 39, except substituting 3-furaldehyde for 3-phenylpropionaldehyde in step a, the title compound was prepared as a clear oil (70%). MS (ESI) 198.2 (M+H)⁺.

### b) furan-3-ylmethyl-(3-[1H-1,2,3-triazol-4-yl]phenyl)amine

Following the procedure of Example 1, except substituting furan-3-ylmethyl-(3-ethynylphenyl)-amine for 3-ethynylphenol, the title compound was prepared as a white solid (20%). ¹H-NMR (400 MHz, CD₃OD): δ 8.06 (s, 1H), 7.49 (s, 1H), 7.45-7.46 (m, 1H), 7.08-7.22 (m, 3H), 6.71 (dd, J=6.5, 1.5 Hz, 1H), 6.47 (s, 1H) 4.22 (s, 2H). MS (ESI) 241.2 (M+H)⁺.

### Example 43

### Preparation of napthalene-1-ylmethyl-(3-[1H-1,2,3-triazol-4-yl]phenyl)amine

### a) napthalene-1-ylmethyl-(3-ethynylphenyl)-amine

Following the procedure of Example 39, except substituting 1-napthaldehyde for 3-phenylpropionaldehyde in step a, the title compound was prepared as a clear oil (80%). MS (ESI) 258.2 (M+H)⁺.

### b) napthalene-1-ylmethyl-(3-[1H-1,2,3-triazol-4-yl]phenyl)amine

Following the procedure of Example 1, except substituting napthalene-1-ylmethyl-(3-ethynylphenyl)-amine for 3-ethynylphenol, the title compound was prepared as a white solid (18%). ¹H-NMR (400 MHz, CD₃OD): δ 8.16 (d, J=8.2 Hz, 1H), 7.99 (br s, 1H), 7.91 (d, J=8.1 Hz, 1H), 7.81 (d, J=8.4 Hz, 1H), 7.42-7.60 (m, 4H), 7.09-7.21 (m, 3H) 6.71 (d, J=8.0 Hz, 1H), 4.83 (s, 2H). MS (ESI) 301.2 (M+H)⁺.

### Example 44

### Preparation of napthalene-2-ylmethyl-(3-[1H-1,2,3-triazol-4-yl]phenyl)amine

### a) napthalene-2-ylmethyl-(3-ethynylphenyl)-amine

Following the procedure of Example 39, except substituting 2-napthaldehyde for 3-phenylpropionaldehyde in step a, the title compound was prepared as a clear oil (90%). MS (ESI) 258.2 (M+H)⁺.

### b) napthalene-2-ylmethyl-(3-[1H-1,2,3-triazol-4-yl]phenyl)amine

Following the procedure of Example 1, except substituting napthalene-2-ylmethyl-(3-ethynylphenyl)-amine for 3-ethynylphenol, the title compound was prepared as a white solid (15%). ¹H-NMR (400 MHz, CD₃OD): δ 8.01 (s, 1H), 7.80-7.87 (m, 4H), 7.41-7.56 (m, 3H), 7.05-7.19 (m, 3H), 6.70 (d, J=1.6 Hz, 1H), 4.56 (s, 2H). MS (ESI) 301.2 (M+H)⁺.

### Example 45

### Preparation of 4-(1H-1,2,3-triazol-4-yl)-phenol

To 4-(4-methoxyphenyl)-1H-1,2,3-triazole (83 mg, 0.5 mmol, from Example 12) was added hydrobromic acid (48% in water, 2 ml) and the solution was heated to 100° C. After three hours, water (10 ml) and ethyl acetate (10 ml) were added. The water layer was washed with ethyl acetate three times and the collected organic layers were dried, filtered, and evaporated. The resulting residue was purified by preparative HPLC to afford the title compound as a white solid (40%). ¹H-NMR (400 MHz, CD₃OD): δ 8.01 (s, 1H), 7.65 (d, J=8.7 Hz, 2H), 6.87 (d, J=8.7 Hz, 2H). MS (ESI) 162.2 (M+H)⁺.

### Example 46

### Preparation of benzyl-(3-[1H-1,2,3-triazol-4-yl]phenyl)amine

To a cooled (0° C.) solution of N-(3-[1H-1,2,3-triazol-4-yl]phenyl)benzamide (50 mg, 0.19 mmol, from Example 6) in THF (0.5 ml) and dioxane (0.5 ml) was added lithium aluminum hydride (1.0 M in THF, 0.2 ml) and the reaction was allowed to warm to room temperature overnight. Additional dioxane (1 ml) and lithium aluminum hydride (0.2 ml) were added with heating to 50° C. to force the reaction to completion. Water and Na₂SO₄ were added and the residue was filtered. The filtrate was evaporated and purified by preparative HPLC to afford the title compound as a tan oil (60%). ¹H-NMR (400 MHz, CD₃OD): δ 7.96 (s, 1H), 7.40-7.43 (m, 2H) 7.30-7.35 (m, 2H), 7.04-7.25 (m, 4H), 6.63 (d, J=8.0 Hz, 1H), 4.38 (s, 2H). MS (ESI) 251.2 (M+H)⁺.

### Example 47

### Preparation of 4-(4-fluorophenyl)-1H-1,2,3-triazole

### a) 1-chloroethynyl-4-fluorobenzene

To a stirring solution of 1-ethynyl-4-fluorobenzene (1.30 g, 10 mmol) in carbon tetrachloride (5 ml) was added potassium carbonate (1.56 g, 11 mmol) and TBAF (0.23 g, 1.0 mmol). After stirring the reaction at RT for 1 h, water (20 ml) was added and the organic material was collected by extraction into chloroform. The combined chloroform extracts were dried (MgSO₄) and evaporated. Purification by silica gel chromatography (100% hexanes) gave the title compound as a clear oil (60%). (This procedure was adapted from Sasson, Y.; Webster, O. W. J. Chem. Soc., Chem. Commun. 1992, 1200.)

### b) 4-fluorophenylethynyltriphenylphosphonium chloride

To triphenylphosphine (1.7 g, 6.3 mmol) in ether (50 ml) was added 1-chloroethynyl-4-fluorobenzene (1.0 g, 6.3 mmol). After sitting for 10 days at RT, the white phosphonium salt was collected by filtration (18%). (This procedure was adapted from Tanaka, Y.; Miller, S. I. J. Org. Chem. 1973, 38, 2708.)

### c) 4-(4-fluorophenyl)-1H-1,2,3-triazole

To a warm (60° C.) solution of sodium azide (74 mg, 1.1 mmol) in DMF (4 ml) was added 4-fluorophenylethynyltriphenylphosphonium chloride (476 mg, 1.1 mmol) in DMF (4 ml) dropwise. After the mixture was stirred for 3 h at 60° C., the DMF was removed by evaporation. The residue was dissolved in chloroform, filtered, and the filtrate was evaporated to give a yellow solid. This solid was dissolved in ethanol (5.5 ml) and a sodium hydroxide solution (0.25 M, 11 ml) was added. After stirring and heating to 90° C. for 2 h, water (20 ml) was added and the aqueous layer was extracted with chloroform (10 ml×2). (The organic layers were discarded.) The aqueous layer was neutralized with HCl (6 N) and again extracted with chloroform (10 ml×3). The organic layers were combined, dried (MgSO₄) and evaporated. Purification by preparative HPLC to afforded the title compound as a yellow solid (20%). ¹H-NMR (400 MHz, CD₃OD): δ 8.13 (s, 1H), 7.84-7.88 (m, 2H), 7.16-7.21 (m, 2H). MS (ESI) 164.2 (M+H)⁺. (This procedure was adapted from Tanaka, Y.; Miller, S. I. J. Org. Chem. 1973, 38, 2708.)

### Example 48

### Preparation of 2-bromo-5-(1-1,2,3-triazolyl)-phenol, 2,6-dibromo-5-(1H-1,2,3-triazol-4-yl)-phenol, and 2,4-dibromo-5-(1H-1,2,3-triazolyl)-phenol

To 3-(1H-1,2,3-triazol-4-yl)-phenol (54 mg, 0.33 mmol, from Example 1) in acetic acid (1 ml) was added bromine (18 uL, 0.33 mmol). After 1 h of stirring at RT, water (10 ml) and ethyl acetate (10 ml) were added. The aqueous layer was neutralized with saturated NaHCO₃. The water layer was washed with ethyl acetate three times and the collected organic layers were dried, filtered, and evaporated. The resulting residue was purified by preparative HPLC to afford the three compounds, each as a white solid. 2-bromo-5-(1H-1,2,3-triazol-4-yl)-phenol (14%): ¹H-NMR (400 MHz, CD₃OD): δ 8.12 (s, 1H), 7.53 (d, J=8.2 Hz, 1H), 7.40 (d, J=2.0 Hz, 1H), 7.22 (dd, J=8.2, 2.0 Hz, 1H). MS (ESI) 240.0 (M+H)⁺. 2,6-dibromo-5-(1H-1,2,3-triazol-4-yl)-phenol (7%): ¹H-NMR (400 MHz, CD₃OD): δ 8.24 (s, 1H), 7.56 (d, J=8.3 Hz, 1H), 7.16 (d, J=8.3 Hz, 1H). MS (ESI) 319.9 (M+H)⁺. 2,4-dibromo-5-(1H-1,2,3-triazol-4-yl)-phenol (8%): ¹H-NMR (400 MHz, CD₃OD): δ 8.29 (s, 1H), 7.80 (s, 1H), 7.36 (s, 1H). MS (ESI) 319.9 (M+H)⁺.

### Example 49

### Preparation of 2-(5-bromo-1H-1,2,3-triazol-4-yl)-4-methyl-pyridine

Following the procedure of Example 48, except substituting 2-(1H-1,2,3-triazol-4-yl)-4-methyl-pyridine (Example 21) for 3-(1H-1,2,3-triazol-4-yl)-phenol, the title compound was prepared as an orange solid (16%). ¹H-NMR (400 MHz, CD₃OD): δ 8.53 (d, J=5.0 Hz, 1H), 7.92 (s, 1H), 7.32 (d, J=5.0 Hz, 1H), 2.48 (s, 3H). MS (ESI) 239.0 (M+H)⁺.

### Example 50

### Preparation of 1H-naptho[1,2-d]-1,2,3-triazole

Morgan, G.; J. Chem. Soc. 1910, 97, 1719. MS (ESI) 170.0 (M+H)⁺.

### Example 51

### Preparation of 2,8-dihydro-indeno[1,2-d]-1,2,3-triazole

Rapoport, H.; Chen, H. H. J. Org. Chem. 1960, 25; 313. MS (ESI) 158.0 (M+H)⁺.

### Example 52

### Preparation of 4-phenyl-1H-1,2,3-triazole

Tanaka, Y.; Velen, S. R.; Miller, S. I. Tetrahedron, 1973, 29, 3271. MS (ESI) 146.0 (M+H)⁺.

### Example 53

### Preparation of 5,5a,6,8-tetrahydro-4H-acenaphtho[4,5-d]-1,2,3-triazole

Rapoport, H.; Nilsson, W. J. Am. Chem. Soc. 1961; 83, 4262. MS (ESI) 198.0 (M+H)⁺.

TABLE I
Provides a three dimensional protein coordinate set of a S. aureus
methionine aminopeptidase crystalline structure.

| Residue Atom | | | X | Y | Z | B |
|---|---|---|---|---|---|---|
| 1 | MET | CB | 6.43 | 53.27 | 9.67 | 15.13 |
| 1 | MET | CG | 6.59 | 52.03 | 10.54 | 15.35 |
| 1 | MET | SD | 5.17 | 51.70 | 11.63 | 15.46 |
| 1 | MET | CE | 5.42 | 52.97 | 12.89 | 15.71 |
| 1 | MET | C | 8.88 | 53.65 | 9.80 | 15.04 |
| 1 | MET | O | 9.76 | 52.80 | 9.72 | 14.99 |
| 1 | MET | N | 7.90 | 52.64 | 7.77 | 14.99 |
| 1 | MET | CA | 7.68 | 53.63 | 8.86 | 15.06 |
| 2 | ILE | N | 8.91 | 54.64 | 10.69 | 15.08 |
| 2 | ILE | CA | 9.98 | 54.75 | 11.68 | 15.12 |
| 2 | ILE | CB | 10.58 | 56.18 | 11.74 | 15.11 |
| 2 | ILE | CG2 | 11.69 | 56.23 | 12.77 | 15.13 |
| 2 | ILE | CG1 | 11.12 | 56.60 | 10.37 | 15.10 |
| 2 | ILE | CD1 | 12.16 | 55.66 | 9.81 | 15.02 |
| 2 | ILE | C | 9.34 | 54.43 | 13.02 | 15.15 |
| 2 | ILE | O | 8.43 | 55.14 | 13.47 | 15.15 |
| 3 | VAL | N | 9.79 | 53.36 | 13.66 | 15.21 |
| 3 | VAL | CA | 9.26 | 52.96 | 14.96 | 15.31 |
| 3 | VAL | CB | 9.75 | 51.54 | 15.37 | 15.33 |
| 3 | VAL | CG1 | 9.22 | 51.17 | 16.74 | 15.35 |
| 3 | VAL | CG2 | 9.31 | 50.51 | 14.33 | 15.31 |
| 3 | VAL | C | 9.71 | 53.94 | 16.03 | 15.41 |
| 3 | VAL | O | 10.90 | 54.19 | 16.20 | 15.43 |
| 4 | LYS | N | 8.74 | 54.51 | 16.75 | 15.50 |
| 4 | LYS | CA | 9.06 | 55.47 | 17.81 | 15.61 |
| 4 | LYS | CB | 8.48 | 56.85 | 17.47 | 15.77 |
| 4 | LYS | CG | 9.22 | 57.62 | 16.36 | 16.09 |
| 4 | LYS | CD | 10.62 | 58.06 | 16.79 | 16.35 |
| 4 | LYS | CE | 11.71 | 57.16 | 16.20 | 16.55 |
| 4 | LYS | NZ | 13.09 | 57.58 | 16.59 | 16.80 |
| 4 | LYS | C | 8.55 | 55.04 | 19.18 | 15.58 |
| 4 | LYS | O | 9.00 | 55.57 | 20.20 | 15.62 |
| 5 | THR | N | 7.61 | 54.11 | 19.21 | 15.49 |
| 5 | THR | CA | 7.06 | 53.66 | 20.49 | 15.51 |
| 5 | THR | CB | 5.63 | 54.18 | 20.71 | 15.51 |
| 5 | THR | OG1 | 4.74 | 53.52 | 19.80 | 15.56 |
| 5 | THR | CG2 | 5.56 | 55.68 | 20.48 | 15.54 |
| 5 | THR | C | 7.02 | 52.14 | 20.63 | 15.55 |
| 5 | THR | O | 7.05 | 51.40 | 19.64 | 15.50 |
| 6 | GLU | N | 6.92 | 51.69 | 21.87 | 15.55 |
| 6 | GLU | CA | 6.86 | 50.27 | 22.20 | 15.56 |
| 6 | GLU | CB | 6.88 | 50.10 | 23.72 | 15.81 |
| 6 | GLU | CG | 6.98 | 48.66 | 24.23 | 16.26 |
| 6 | GLU | CD | 8.35 | 48.06 | 24.03 | 16.49 |
| 6 | GLU | OE1 | 9.36 | 48.76 | 24.27 | 16.76 |
| 6 | GLU | OE2 | 8.43 | 46.87 | 23.65 | 16.71 |
| 6 | GLU | C | 5.58 | 49.68 | 21.61 | 15.27 |
| 6 | GLU | O | 5.54 | 48.53 | 21.17 | 15.37 |
| 7 | GLU | N | 4.51 | 50.48 | 21.62 | 15.00 |
| 7 | GLU | CA | 3.22 | 50.03 | 21.09 | 14.67 |
| 7 | GLU | CB | 2.14 | 51.09 | 21.34 | 14.95 |
| 7 | GLU | CG | 1.69 | 51.24 | 22.80 | 15.30 |
| 7 | GLU | CD | 2.77 | 51.79 | 23.74 | 15.51 |
| 7 | GLU | OE1 | 3.57 | 52.66 | 23.31 | 15.59 |
| 7 | GLU | OE2 | 2.81 | 51.36 | 24.91 | 15.76 |
| 7 | GLU | C | 3.30 | 49.72 | 19.59 | 14.29 |
| 7 | GLU | O | 2.76 | 48.72 | 19.12 | 14.21 |
| 8 | GLU | N | 3.99 | 50.58 | 18.84 | 13.86 |
| 8 | GLU | CA | 4.12 | 50.37 | 17.40 | 13.29 |
| 8 | GLU | CB | 4.80 | 51.58 | 16.75 | 13.39 |
| 8 | GLU | CG | 4.06 | 52.88 | 16.97 | 13.55 |
| 8 | GLU | CD | 4.73 | 54.08 | 16.32 | 13.59 |
| 8 | GLU | OE1 | 5.97 | 54.11 | 16.27 | 13.66 |
| 8 | GLU | OE2 | 4.01 | 54.99 | 15.86 | 13.76 |
| 8 | GLU | C | 4.96 | 49.12 | 17.16 | 12.88 |
| 8 | GLU | O | 4.65 | 48.31 | 16.29 | 12.87 |
| 9 | LEU | N | 6.02 | 48.96 | 17.95 | 12.40 |
| 9 | LEU | CA | 6.88 | 47.80 | 17.82 | 12.00 |
| 9 | LEU | CB | 8.04 | 47.86 | 18.81 | 12.11 |
| 9 | LEU | CG | 8.95 | 46.63 | 18.86 | 12.20 |
| 9 | LEU | CD1 | 9.63 | 46.43 | 17.52 | 12.27 |
| 9 | LEU | CD2 | 9.99 | 46.81 | 19.96 | 12.19 |
| 9 | LEU | C | 6.06 | 46.52 | 18.04 | 11.68 |
| 9 | LEU | O | 6.15 | 45.57 | 17.27 | 11.59 |
| 10 | GLN | N | 5.27 | 46.51 | 19.10 | 11.43 |
| 10 | GLN | CA | 4.45 | 45.33 | 19.40 | 11.04 |
| 10 | GLN | CB | 3.73 | 45.52 | 20.74 | 11.15 |
| 10 | GLN | CG | 4.68 | 45.90 | 21.87 | 11.40 |
| 10 | GLN | CD | 3.96 | 46.15 | 23.18 | 11.56 |
| 10 | GLN | OE1 | 2.77 | 46.45 | 23.19 | 11.74 |
| 10 | GLN | NE2 | 4.70 | 46.06 | 24.28 | 11.68 |
| 10 | GLN | C | 3.44 | 45.08 | 18.29 | 10.68 |
| 10 | GLN | O | 3.20 | 43.93 | 17.92 | 10.61 |
| 11 | ALA | N | 2.86 | 46.14 | 17.75 | 10.23 |
| 11 | ALA | CA | 1.89 | 45.99 | 16.67 | 9.81 |
| 11 | ALA | CB | 1.28 | 47.34 | 16.32 | 9.92 |
| 11 | ALA | C | 2.54 | 45.38 | 15.44 | 9.56 |
| 11 | ALA | O | 1.95 | 44.54 | 14.76 | 9.43 |
| 12 | LEU | N | 3.78 | 45.78 | 15.16 | 9.18 |
| 12 | LEU | CA | 4.50 | 45.26 | 14.01 | 8.86 |
| 12 | LEU | CB | 5.76 | 46.10 | 13.75 | 8.81 |
| 12 | LEU | CG | 5.45 | 47.50 | 13.19 | 8.76 |
| 12 | LEU | CD1 | 6.69 | 48.40 | 13.25 | 8.74 |
| 12 | LEU | CD2 | 4.98 | 47.35 | 11.75 | 8.67 |
| 12 | LEU | C | 4.88 | 43.80 | 14.22 | 8.73 |
| 12 | LEU | O | 4.74 | 42.97 | 13.32 | 8.65 |
| 13 | LYS | N | 5.35 | 43.47 | 15.42 | 8.53 |
| 13 | LYS | CA | 5.71 | 42.10 | 15.72 | 8.41 |
| 13 | LYS | CB | 6.34 | 42.02 | 17.11 | 8.63 |
| 13 | LYS | CG | 7.72 | 42.62 | 17.18 | 8.98 |
| 13 | LYS | CD | 8.24 | 42.59 | 18.61 | 9.38 |
| 13 | LYS | CE | 9.70 | 42.96 | 18.66 | 9.70 |
| 13 | LYS | NZ | 10.17 | 42.99 | 20.08 | 10.07 |
| 13 | LYS | C | 4.48 | 41.21 | 15.66 | 8.24 |
| 13 | LYS | O | 4.56 | 40.05 | 15.23 | 8.19 |
| 14 | GLU | N | 3.34 | 41.74 | 16.07 | 8.06 |
| 14 | GLU | CA | 2.10 | 40.96 | 16.04 | 7.92 |
| 14 | GLU | CB | 0.94 | 41.75 | 16.67 | 8.16 |
| 14 | GLU | CG | −0.45 | 41.11 | 16.50 | 8.49 |
| 14 | GLU | CD | −0.64 | 39.84 | 17.32 | 8.63 |
| 14 | GLU | OE1 | 0.26 | 39.45 | 18.09 | 8.80 |
| 14 | GLU | OE2 | −1.72 | 39.23 | 17.21 | 8.81 |
| 14 | GLU | C | 1.74 | 40.54 | 14.62 | 7.74 |
| 14 | GLU | O | 1.61 | 39.35 | 14.33 | 7.61 |
| 15 | ILE | N | 1.60 | 41.51 | 13.72 | 7.57 |
| 15 | ILE | CA | 1.23 | 41.20 | 12.34 | 7.36 |
| 15 | ILE | CB | 0.85 | 42.48 | 11.54 | 7.27 |
| 15 | ILE | CG2 | 2.04 | 43.45 | 11.48 | 7.26 |
| 15 | ILE | CG1 | 0.35 | 42.10 | 10.15 | 7.18 |
| 15 | ILE | CD1 | −0.96 | 41.35 | 10.15 | 7.09 |
| 15 | ILE | C | 2.34 | 40.42 | 11.62 | 7.32 |
| 15 | ILE | O | 2.07 | 39.62 | 10.73 | 7.18 |
| 16 | GLY | N | 3.58 | 40.65 | 12.04 | 7.28 |
| 16 | GLY | CA | 4.70 | 39.94 | 11.45 | 7.07 |
| 16 | GLY | C | 4.58 | 38.46 | 11.77 | 6.91 |
| 16 | GLY | O | 4.76 | 37.61 | 10.89 | 7.16 |
| 17 | TYR | N | 4.25 | 38.13 | 13.02 | 6.79 |
| 17 | TYR | CA | 4.10 | 36.74 | 13.43 | 6.80 |
| 17 | TYR | CB | 3.75 | 36.62 | 14.91 | 7.14 |
| 17 | TYR | CG | 3.40 | 35.19 | 15.31 | 7.09 |
| 17 | TYR | CD1 | 4.39 | 34.22 | 15.47 | 7.20 |
| 17 | TYR | CE1 | 4.05 | 32.87 | 15.71 | 7.37 |
| 17 | TYR | CD2 | 2.06 | 34.78 | 15.41 | 7.25 |
| 17 | TYR | CE2 | 1.72 | 33.45 | 15.64 | 7.33 |
| 17 | TYR | CZ | 2.71 | 32.51 | 15.79 | 7.40 |
| 17 | TYR | OH | 2.38 | 31.19 | 16.01 | 7.56 |
| 17 | TYR | C | 2.98 | 36.08 | 12.63 | 6.75 |
| 17 | TYR | O | 3.12 | 34.95 | 12.16 | 6.70 |
| 18 | ILE | N | 1.86 | 36.77 | 12.52 | 6.69 |
| 18 | ILE | CA | 0.71 | 36.24 | 11.81 | 6.68 |
| 18 | ILE | CB | −0.45 | 37.25 | 11.81 | 6.72 |
| 18 | ILE | CG2 | −1.61 | 36.74 | 10.94 | 6.69 |
| 18 | ILE | CG1 | −0.95 | 37.44 | 13.25 | 6.80 |
| 18 | ILE | CD1 | −2.00 | 38.55 | 13.40 | 6.98 |
| 18 | ILE | C | 1.05 | 35.88 | 10.37 | 6.67 |
| 18 | ILE | O | 0.75 | 34.76 | 9.92 | 6.57 |
| 19 | CYS | N | 1.70 | 36.79 | 9.66 | 6.72 |
| 19 | CYS | CA | 2.06 | 36.50 | 8.27 | 6.84 |
| 19 | CYS | CB | 2.62 | 37.75 | 7.58 | 7.20 |
| 19 | CYS | SG | 1.39 | 39.09 | 7.40 | 7.57 |
| 19 | CYS | C | 3.06 | 35.34 | 8.16 | 6.71 |
| 19 | CYS | O | 2.97 | 34.52 | 7.25 | 6.49 |
| 20 | ALA | N | 4.00 | 35.28 | 9.10 | 6.63 |
| 20 | ALA | CA | 4.98 | 34.21 | 9.10 | 6.57 |
| 20 | ALA | CB | 6.04 | 34.45 | 10.19 | 6.67 |
| 20 | ALA | C | 4.28 | 32.87 | 9.35 | 6.54 |
| 20 | ALA | O | 4.58 | 31.86 | 8.70 | 6.52 |
| 21 | LYS | N | 3.34 | 32.88 | 10.28 | 6.45 |
| 21 | LYS | CA | 2.58 | 31.67 | 10.61 | 6.56 |
| 21 | LYS | CB | 1.61 | 31.94 | 11.76 | 6.69 |
| 21 | LYS | CG | 0.93 | 30.69 | 12.33 | 7.10 |
| 21 | LYS | CD | 1.97 | 29.70 | 12.86 | 7.42 |
| 21 | LYS | CE | 1.34 | 28.55 | 13.62 | 7.70 |
| 21 | LYS | NZ | 2.39 | 27.66 | 14.18 | 7.91 |
| 21 | LYS | C | 1.81 | 31.19 | 9.39 | 6.45 |
| 21 | LYS | O | 1.78 | 30.00 | 9.08 | 6.36 |
| 22 | VAL | N | 1.17 | 32.13 | 8.71 | 6.45 |
| 22 | VAL | CA | 0.39 | 31.80 | 7.52 | 6.41 |
| 22 | VAL | CB | −0.35 | 33.04 | 7.00 | 6.34 |
| 22 | VAL | CG1 | −0.99 | 32.75 | 5.64 | 6.41 |
| 22 | VAL | CG2 | −1.41 | 33.44 | 8.01 | 6.53 |
| 22 | VAL | C | 1.31 | 31.23 | 6.44 | 6.40 |
| 22 | VAL | O | 1.03 | 30.19 | 5.83 | 6.33 |
| 23 | ARG | N | 2.41 | 31.94 | 6.20 | 6.40 |
| 23 | ARG | CA | 3.40 | 31.53 | 5.21 | 6.30 |
| 23 | ARG | CB | 4.58 | 32.51 | 5.24 | 6.19 |
| 23 | ARG | CG | 5.63 | 32.31 | 4.16 | 6.30 |
| 23 | ARG | CD | 6.93 | 32.94 | 4.62 | 6.30 |
| 23 | ARG | NE | 7.45 | 32.23 | 5.79 | 6.30 |
| 23 | ARG | CZ | 8.05 | 32.81 | 6.83 | 6.36 |
| 23 | ARG | NH1 | 8.21 | 34.12 | 6.86 | 6.29 |
| 23 | ARG | NH2 | 8.46 | 32.07 | 7.85 | 6.22 |
| 23 | ARG | C | 3.89 | 30.10 | 5.49 | 6.27 |
| 23 | ARG | O | 3.95 | 29.26 | 4.58 | 6.26 |
| 24 | ASN | N | 4.24 | 29.81 | 6.75 | 6.41 |
| 24 | ASN | CA | 4.70 | 28.46 | 7.09 | 6.60 |
| 24 | ASN | CB | 5.18 | 28.39 | 8.54 | 6.95 |
| 24 | ASN | CG | 6.48 | 29.13 | 8.77 | 7.09 |
| 24 | ASN | OD1 | 7.25 | 29.36 | 7.84 | 7.26 |
| 24 | ASN | ND2 | 6.74 | 29.50 | 10.02 | 7.37 |
| 24 | ASN | C | 3.62 | 27.40 | 6.89 | 6.59 |
| 24 | ASN | O | 3.90 | 26.32 | 6.36 | 6.64 |
| 25 | THR | N | 2.39 | 27.71 | 7.31 | 6.71 |
| 25 | THR | CA | 1.30 | 26.76 | 7.18 | 6.79 |
| 25 | THR | CB | 0.03 | 27.28 | 7.91 | 6.77 |
| 25 | THR | OG1 | 0.36 | 27.63 | 9.26 | 6.87 |
| 25 | THR | CG2 | −1.05 | 26.20 | 7.95 | 6.91 |
| 25 | THR | C | 1.00 | 26.46 | 5.70 | 6.87 |
| 25 | THR | O | 0.82 | 25.30 | 5.32 | 6.68 |
| 26 | MET | N | 0.97 | 27.50 | 4.87 | 7.05 |
| 26 | MET | CA | 0.73 | 27.32 | 3.44 | 7.14 |
| 26 | MET | CB | 0.60 | 28.67 | 2.73 | 7.12 |
| 26 | MET | CG | −0.64 | 29.44 | 3.15 | 7.34 |
| 26 | MET | SD | −0.79 | 31.09 | 2.43 | 7.05 |
| 26 | MET | CE | −1.04 | 30.70 | 0.73 | 7.35 |
| 26 | MET | C | 1.84 | 26.50 | 2.78 | 7.12 |
| 26 | MET | O | 1.57 | 25.60 | 1.98 | 7.04 |
| 27 | GLN | N | 3.08 | 26.81 | 3.12 | 7.19 |
| 27 | GLN | CA | 4.20 | 26.09 | 2.54 | 7.31 |
| 27 | GLN | CB | 5.54 | 26.65 | 3.04 | 7.31 |
| 27 | GLN | CG | 6.75 | 25.87 | 2.52 | 7.37 |
| 27 | GLN | CD | 8.06 | 26.55 | 2.83 | 7.41 |
| 27 | GLN | OE1 | 8.10 | 27.51 | 3.58 | 7.44 |
| 27 | GLN | NE2 | 9.15 | 26.04 | 2.26 | 7.41 |
| 27 | GLN | C | 4.14 | 24.59 | 2.85 | 7.46 |
| 27 | GLN | O | 4.31 | 23.75 | 1.95 | 7.45 |
| 28 | ALA | N | 3.90 | 24.26 | 4.12 | 7.61 |
| 28 | ALA | CA | 3.83 | 22.88 | 4.56 | 7.74 |
| 28 | ALA | CB | 3.62 | 22.83 | 6.07 | 7.64 |
| 28 | ALA | C | 2.71 | 22.12 | 3.87 | 7.82 |
| 28 | ALA | O | 2.79 | 20.91 | 3.67 | 7.93 |
| 29 | ALA | N | 1.66 | 22.84 | 3.49 | 8.04 |
| 29 | ALA | CA | 0.52 | 22.25 | 2.82 | 8.20 |
| 29 | ALA | CB | −0.76 | 23.01 | 3.20 | 8.31 |
| 29 | ALA | C | 0.65 | 22.18 | 1.30 | 8.39 |
| 29 | ALA | O | −0.20 | 21.59 | 0.62 | 8.33 |
| 30 | THR | N | 1.71 | 22.79 | 0.76 | 8.61 |
| 30 | THR | CA | 1.92 | 22.79 | −0.68 | 8.69 |
| 30 | THR | CB | 2.86 | 23.95 | −1.13 | 8.69 |
| 30 | THR | OG1 | 2.30 | 25.20 | −0.70 | 8.77 |
| 30 | THR | CG2 | 2.98 | 23.99 | −2.66 | 8.72 |
| 30 | THR | C | 2.50 | 21.45 | −1.15 | 8.82 |
| 30 | THR | O | 3.71 | 21.21 | −1.05 | 8.85 |
| 31 | LYS | N | 1.63 | 20.59 | −1.65 | 8.92 |
| 31 | LYS | CA | 2.02 | 19.28 | −2.16 | 8.94 |
| 31 | LYS | CB | 1.62 | 18.18 | −1.16 | 9.33 |
| 31 | LYS | CG | 0.13 | 18.13 | −0.84 | 9.68 |
| 31 | LYS | CD | −0.21 | 16.89 | −0.01 | 10.00 |
| 31 | LYS | CE | 0.05 | 15.61 | −0.82 | 10.30 |
| 31 | LYS | NZ | 0.14 | 14.40 | 0.02 | 10.46 |
| 31 | LYS | C | 1.31 | 19.02 | −3.48 | 9.07 |
| 31 | LYS | O | 0.33 | 19.70 | −3.82 | 9.07 |
| 32 | PRO | N | 1.79 | 18.05 | −4.27 | 9.23 |
| 32 | PRO | CD | 3.05 | 17.29 | −4.12 | 9.24 |
| 32 | PRO | CA | 1.14 | 17.75 | −5.55 | 9.25 |
| 32 | PRO | CB | 1.94 | 16.56 | −6.07 | 9.39 |
| 32 | PRO | CG | 3.32 | 16.83 | −5.53 | 9.26 |
| 32 | PRO | C | −0.34 | 17.41 | −5.32 | 9.45 |
| 32 | PRO | O | −0.68 | 16.70 | −4.38 | 9.64 |
| 33 | GLY | N | −1.21 | 17.93 | −6.17 | 9.66 |
| 33 | GLY | CA | −2.62 | 17.63 | −6.03 | 9.78 |
| 33 | GLY | C | −3.44 | 18.72 | −5.38 | 9.77 |
| 33 | GLY | O | −4.67 | 18.73 | −5.49 | 9.99 |
| 34 | ILE | N | −2.80 | 19.65 | −4.68 | 9.59 |
| 34 | ILE | CA | −3.55 | 20.73 | −4.06 | 9.39 |
| 34 | ILE | CB | −2.89 | 21.19 | −2.73 | 9.22 |
| 34 | ILE | CG2 | −1.56 | 21.86 | −3.00 | 9.10 |
| 34 | ILE | CG1 | −3.83 | 22.14 | −1.99 | 9.11 |
| 34 | ILE | CD1 | −3.39 | 22.44 | −0.59 | 9.02 |
| 34 | ILE | C | −3.62 | 21.89 | −5.05 | 9.25 |
| 34 | ILE | O | −2.71 | 22.05 | −5.87 | 9.40 |
| 35 | THR | N | −4.69 | 22.67 | −5.03 | 9.25 |
| 35 | THR | CA | −4.78 | 23.80 | −5.95 | 9.22 |
| 35 | THR | CB | −6.21 | 24.06 | −6.46 | 9.19 |
| 35 | THR | OG1 | −7.03 | 24.54 | −5.38 | 9.18 |
| 35 | THR | CG2 | −6.82 | 22.79 | −7.05 | 9.18 |
| 35 | THR | C | −4.29 | 25.05 | −5.23 | 9.07 |
| 35 | THR | O | −4.30 | 25.09 | −4.00 | 9.07 |
| 36 | THR | N | −3.89 | 26.06 | −5.98 | 9.13 |
| 36 | THR | CA | −3.42 | 27.27 | −5.34 | 9.25 |
| 36 | THR | CB | −2.79 | 28.26 | −6.36 | 9.29 |
| 36 | THR | OG1 | −3.74 | 28.59 | −7.38 | 9.20 |
| 36 | THR | CG2 | −1.55 | 27.64 | −7.00 | 9.23 |
| 36 | THR | C | −4.55 | 27.95 | −4.59 | 9.23 |
| 36 | THR | O | −4.31 | 28.60 | −3.58 | 9.32 |
| 37 | LYS | N | −5.79 | 27.78 | −5.05 | 9.33 |
| 37 | LYS | CA | −6.91 | 28.40 | −4.35 | 9.43 |
| 37 | LYS | CB | −8.20 | 28.30 | −5.18 | 9.55 |
| 37 | LYS | CG | −9.33 | 29.19 | −4.64 | 9.66 |
| 37 | LYS | CD | −8.88 | 30.64 | −4.56 | 9.79 |
| 37 | LYS | CE | −9.81 | 31.50 | −3.72 | 9.85 |
| 37 | LYS | NZ | −9.20 | 32.86 | −3.54 | 9.91 |
| 37 | LYS | C | −7.10 | 27.72 | −2.99 | 9.46 |
| 37 | LYS | O | −7.44 | 28.38 | −2.01 | 9.43 |
| 38 | GLU | N | −6.88 | 26.41 | −2.92 | 9.48 |
| 38 | GLU | CA | −7.01 | 25.70 | −1.66 | 9.49 |
| 38 | GLU | CB | −6.88 | 24.19 | −1.86 | 9.76 |
| 38 | GLU | CG | −8.02 | 23.62 | −2.69 | 10.29 |
| 38 | GLU | CD | −7.93 | 22.11 | −2.84 | 10.65 |
| 38 | GLU | OE1 | −6.95 | 21.62 | −3.43 | 10.83 |
| 38 | GLU | OE2 | −8.86 | 21.43 | −2.37 | 11.08 |
| 38 | GLU | C | −5.96 | 26.20 | −0.67 | 9.31 |
| 38 | GLU | O | −6.22 | 26.27 | 0.54 | 9.17 |
| 39 | LEU | N | −4.78 | 26.52 | −1.18 | 9.18 |
| 39 | LEU | CA | −3.71 | 27.04 | −0.33 | 9.06 |
| 39 | LEU | CB | −2.39 | 27.15 | −1.11 | 9.16 |
| 39 | LEU | CG | −1.68 | 25.85 | −1.47 | 9.23 |
| 39 | LEU | CD1 | −0.46 | 26.16 | −2.33 | 9.29 |
| 39 | LEU | CD2 | −1.25 | 25.12 | −0.19 | 9.25 |
| 39 | LEU | C | −4.10 | 28.43 | 0.17 | 8.98 |
| 39 | LEU | O | −3.87 | 28.77 | 1.32 | 8.92 |
| 40 | ASP | N | −4.71 | 29.21 | −0.71 | 8.97 |
| 40 | ASP | CA | −5.14 | 30.56 | −0.36 | 8.93 |
| 40 | ASP | CB | −5.69 | 31.27 | −1.61 | 8.89 |
| 40 | ASP | CG | −5.87 | 32.75 | −1.40 | 8.96 |
| 40 | ASP | OD1 | −4.86 | 33.45 | −1.16 | 8.83 |
| 40 | ASP | OD2 | −7.02 | 33.23 | −1.46 | 9.04 |
| 40 | ASP | C | −6.21 | 30.49 | 0.72 | 8.94 |
| 40 | ASP | O | −6.27 | 31.36 | 1.59 | 8.90 |
| 41 | ASN | N | −7.04 | 29.45 | 0.69 | 9.03 |
| 41 | ASN | CA | −8.10 | 29.29 | 1.68 | 9.17 |
| 41 | ASN | CB | −9.00 | 28.12 | 1.31 | 9.22 |
| 41 | ASN | CG | −9.87 | 28.42 | 0.11 | 9.40 |
| 41 | ASN | OD1 | −10.04 | 29.59 | −0.25 | 9.51 |
| 41 | ASN | ND2 | −10.42 | 27.38 | −0.51 | 9.36 |
| 41 | ASN | C | −7.56 | 29.11 | 3.09 | 9.23 |
| 41 | ASN | O | −8.23 | 29.43 | 4.07 | 9.17 |
| 42 | ILE | N | −6.35 | 28.57 | 3.18 | 9.33 |
| 42 | ILE | CA | −5.71 | 28.38 | 4.46 | 9.41 |
| 42 | ILE | CB | −4.37 | 27.62 | 4.31 | 9.26 |
| 42 | ILE | CG2 | −3.66 | 27.53 | 5.66 | 9.31 |
| 42 | ILE | CG1 | −4.64 | 26.23 | 3.75 | 9.29 |
| 42 | ILE | CD1 | −3.38 | 25.43 | 3.45 | 9.19 |
| 42 | ILE | C | −5.44 | 29.77 | 5.04 | 9.55 |
| 42 | ILE | O | −5.73 | 30.02 | 6.21 | 9.59 |
| 43 | ALA | N | −4.91 | 30.67 | 4.22 | 9.69 |
| 43 | ALA | CA | −4.63 | 32.03 | 4.68 | 9.85 |
| 43 | ALA | CB | −3.96 | 32.84 | 3.57 | 9.88 |
| 43 | ALA | C | −5.92 | 32.70 | 5.12 | 9.99 |
| 43 | ALA | O | −5.96 | 33.39 | 6.14 | 9.97 |
| 44 | LYS | N | −6.99 | 32.51 | 4.34 | 10.19 |
| 44 | LYS | CA | −8.28 | 33.11 | 4.69 | 10.53 |
| 44 | LYS | CB | −9.35 | 32.66 | 3.69 | 10.58 |
| 44 | LYS | CG | −10.77 | 33.15 | 4.01 | 10.83 |
| 44 | LYS | CD | −11.79 | 32.53 | 3.06 | 11.07 |
| 44 | LYS | CE | −13.20 | 33.03 | 3.33 | 11.32 |
| 44 | LYS | NZ | −13.30 | 34.51 | 3.20 | 11.65 |
| 44 | LYS | C | −8.70 | 32.73 | 6.12 | 10.64 |
| 44 | LYS | O | −9.05 | 33.59 | 6.92 | 10.64 |
| 45 | GLU | N | −8.64 | 31.44 | 6.44 | 10.98 |
| 45 | GLU | CA | −9.04 | 30.98 | 7.78 | 11.30 |
| 45 | GLU | CB | −9.12 | 29.45 | 7.83 | 11.78 |
| 45 | GLU | CG | −10.14 | 28.84 | 6.88 | 12.72 |
| 45 | GLU | CD | −11.54 | 29.44 | 7.04 | 13.19 |
| 45 | GLU | OE1 | −12.03 | 29.55 | 8.18 | 13.63 |
| 45 | GLU | OE2 | −12.15 | 29.79 | 6.00 | 13.66 |
| 45 | GLU | C | −8.11 | 31.46 | 8.89 | 11.24 |
| 45 | GLU | O | −8.58 | 31.79 | 9.99 | 11.18 |
| 46 | LEU | N | −6.81 | 31.49 | 8.63 | 11.18 |
| 46 | LEU | CA | −5.87 | 31.94 | 9.65 | 11.19 |
| 46 | LEU | CB | −4.43 | 31.57 | 9.26 | 11.17 |
| 46 | LEU | CG | −4.09 | 30.07 | 9.34 | 11.24 |
| 46 | LEU | CD1 | −2.63 | 29.86 | 8.93 | 11.21 |
| 46 | LEU | CD2 | −4.32 | 29.56 | 10.75 | 11.23 |
| 46 | LEU | C | −5.99 | 33.44 | 9.87 | 11.23 |
| 46 | LEU | O | −5.95 | 33.91 | 11.02 | 11.22 |
| 47 | PHE | N | −6.14 | 34.21 | 8.80 | 11.24 |
| 47 | PHE | CA | −6.28 | 35.66 | 8.97 | 11.27 |
| 47 | PHE | CB | −6.46 | 36.38 | 7.63 | 10.95 |
| 47 | PHE | CG | −5.20 | 36.51 | 6.81 | 10.75 |
| 47 | PHE | CD1 | −3.95 | 36.55 | 7.42 | 10.60 |
| 47 | PHE | CD2 | −5.28 | 36.62 | 5.43 | 10.58 |
| 47 | PHE | CE1 | −2.78 | 36.71 | 6.64 | 10.51 |
| 47 | PHE | CE2 | −4.13 | 36.77 | 4.65 | 10.51 |
| 47 | PHE | CZ | −2.88 | 36.82 | 5.25 | 10.46 |
| 47 | PHE | C | −7.48 | 35.94 | 9.87 | 11.40 |
| 47 | PHE | O | −7.41 | 36.79 | 10.76 | 11.37 |
| 48 | GLU | N | −8.57 | 35.22 | 9.65 | 11.75 |
| 48 | GLU | CA | −9.77 | 35.40 | 10.46 | 12.12 |
| 48 | GLU | CB | −10.91 | 34.56 | 9.89 | 12.54 |
| 48 | GLU | CG | −12.21 | 34.62 | 10.68 | 13.33 |
| 48 | GLU | CD | −12.76 | 36.03 | 10.79 | 13.70 |
| 48 | GLU | OE1 | −12.83 | 36.73 | 9.75 | 14.19 |
| 48 | GLU | OE2 | −13.15 | 36.43 | 11.91 | 14.10 |
| 48 | GLU | C | −9.52 | 35.02 | 11.92 | 12.12 |
| 48 | GLU | O | −9.94 | 35.72 | 12.84 | 12.16 |
| 49 | GLU | N | −8.81 | 33.91 | 12.11 | 12.32 |
| 49 | GLU | CA | −8.48 | 33.41 | 13.45 | 12.49 |
| 49 | GLU | CB | −7.77 | 32.06 | 13.32 | 12.89 |
| 49 | GLU | CG | −7.43 | 31.38 | 14.65 | 13.71 |
| 49 | GLU | CD | −6.86 | 29.98 | 14.46 | 14.11 |
| 49 | GLU | OE1 | −7.56 | 29.12 | 13.88 | 14.52 |
| 49 | GLU | OE2 | −5.71 | 29.74 | 14.88 | 14.52 |
| 49 | GLU | C | −7.61 | 34.38 | 14.25 | 12.42 |
| 49 | GLU | O | −7.82 | 34.57 | 15.45 | 12.25 |
| 50 | TYR | N | −6.62 | 34.99 | 13.59 | 12.35 |
| 50 | TYR | CA | −5.71 | 35.93 | 14.24 | 12.25 |
| 50 | TYR | CB | −4.32 | 35.83 | 13.62 | 12.28 |
| 50 | TYR | CG | −3.61 | 34.53 | 13.91 | 12.54 |
| 50 | TYR | CD1 | −3.05 | 34.28 | 15.16 | 12.57 |
| 50 | TYR | CE1 | −2.40 | 33.08 | 15.44 | 12.69 |
| 50 | TYR | CD2 | −3.52 | 33.53 | 12.94 | 12.58 |
| 50 | TYR | CE2 | −2.87 | 32.33 | 13.21 | 12.66 |
| 50 | TYR | CZ | −2.32 | 32.11 | 14.45 | 12.71 |
| 50 | TYR | OH | −1.68 | 30.92 | 14.70 | 12.87 |
| 50 | TYR | C | −6.17 | 37.39 | 14.20 | 12.14 |
| 50 | TYR | O | −5.47 | 38.27 | 14.70 | 12.18 |
| 51 | GLY | N | −7.32 | 37.64 | 13.59 | 11.95 |
| 51 | GLY | CA | −7.83 | 38.99 | 13.52 | 11.69 |
| 51 | GLY | C | −7.09 | 39.92 | 12.58 | 11.51 |
| 51 | GLY | O | −7.02 | 41.13 | 12.83 | 11.49 |
| 52 | ALA | N | −6.54 | 39.37 | 11.51 | 11.26 |
| 52 | ALA | CA | −5.82 | 40.16 | 10.52 | 10.96 |
| 52 | ALA | CB | −4.43 | 39.57 | 10.29 | 10.92 |
| 52 | ALA | C | −6.59 | 40.18 | 9.21 | 10.80 |
| 52 | ALA | O | −7.52 | 39.40 | 9.00 | 10.69 |
| 53 | ILE | N | −6.19 | 41.08 | 8.32 | 10.74 |
| 53 | ILE | CA | −6.83 | 41.22 | 7.03 | 10.76 |
| 53 | ILE | CB | −7.59 | 42.55 | 6.93 | 11.09 |
| 53 | ILE | CG2 | −8.12 | 42.75 | 5.54 | 11.08 |
| 53 | ILE | CG1 | −8.74 | 42.55 | 7.94 | 11.34 |
| 53 | ILE | CD1 | −9.71 | 41.43 | 7.75 | 11.84 |
| 53 | ILE | C | −5.82 | 41.18 | 5.90 | 10.65 |
| 53 | ILE | O | −4.71 | 41.70 | 6.02 | 10.56 |
| 54 | SER | N | −6.21 | 40.55 | 4.80 | 10.57 |
| 54 | SER | CA | −5.36 | 40.43 | 3.61 | 10.72 |
| 54 | SER | CB | −6.10 | 39.63 | 2.54 | 10.72 |
| 54 | SER | OG | −5.47 | 39.78 | 1.27 | 10.63 |
| 54 | SER | C | −4.99 | 41.80 | 3.04 | 10.77 |
| 54 | SER | O | −5.88 | 42.60 | 2.74 | 10.76 |
| 55 | ALA | N | −3.70 | 42.08 | 2.87 | 10.90 |
| 55 | ALA | CA | −3.29 | 43.36 | 2.31 | 11.05 |
| 55 | ALA | CB | −1.80 | 43.61 | 2.54 | 10.96 |
| 55 | ALA | C | −3.61 | 43.45 | 0.81 | 11.16 |
| 55 | ALA | O | −4.05 | 44.48 | 0.34 | 11.10 |
| 56 | PRO | N | −3.39 | 42.36 | 0.05 | 11.37 |
| 56 | PRO | CD | −2.66 | 41.12 | 0.38 | 11.33 |
| 56 | PRO | CA | −3.69 | 42.41 | −1.38 | 11.44 |
| 56 | PRO | CB | −3.42 | 40.98 | −1.85 | 11.44 |
| 56 | PRO | CG | −2.25 | 40.60 | −1.01 | 11.38 |
| 56 | PRO | C | −5.14 | 42.82 | −1.62 | 11.71 |
| 56 | PRO | O | −5.44 | 43.65 | −2.48 | 11.70 |
| 57 | ILE | N | −6.04 | 42.23 | −0.84 | 11.87 |
| 57 | ILE | CA | −7.46 | 42.53 | −0.97 | 12.01 |
| 57 | ILE | CB | −8.32 | 41.55 | −0.16 | 12.02 |
| 57 | ILE | CG2 | −9.77 | 42.06 | −0.11 | 12.05 |
| 57 | ILE | CG1 | −8.23 | 40.16 | −0.79 | 11.93 |
| 57 | ILE | CD1 | −8.96 | 39.08 | 0.01 | 11.78 |
| 57 | ILE | C | −7.82 | 43.94 | −0.49 | 12.22 |
| 57 | ILE | O | −8.53 | 44.67 | −1.17 | 12.22 |
| 58 | HIS | N | −7.32 | 44.30 | 0.69 | 12.37 |
| 58 | HIS | CA | −7.60 | 45.60 | 1.28 | 12.63 |
| 58 | HIS | CB | −7.13 | 45.60 | 2.74 | 12.73 |
| 58 | HIS | CG | −7.25 | 46.94 | 3.42 | 12.73 |
| 58 | HIS | CD2 | −8.22 | 47.44 | 4.22 | 12.78 |
| 58 | HIS | ND1 | −6.29 | 47.92 | 3.31 | 12.80 |
| 58 | HIS | CE1 | −6.66 | 48.97 | 4.02 | 12.82 |
| 58 | HIS | NE2 | −7.82 | 48.71 | 4.58 | 12.84 |
| 58 | HIS | C | −6.97 | 46.77 | 0.53 | 12.81 |
| 58 | HIS | O | −7.62 | 47.79 | 0.32 | 12.82 |
| 59 | ASP | N | −5.71 | 46.60 | 0.12 | 12.96 |
| 59 | ASP | CA | −4.94 | 47.63 | −0.58 | 13.19 |
| 59 | ASP | CB | −3.44 | 47.41 | −0.39 | 13.25 |
| 59 | ASP | CG | −2.98 | 47.57 | 1.05 | 13.42 |
| 59 | ASP | OD1 | −3.78 | 47.95 | 1.92 | 13.43 |
| 59 | ASP | OD2 | −1.77 | 47.33 | 1.29 | 13.51 |
| 59 | ASP | C | −5.16 | 47.77 | −2.08 | 13.27 |
| 59 | ASP | O | −5.28 | 48.89 | −2.59 | 13.39 |
| 60 | GLU | N | −5.16 | 46.65 | −2.80 | 13.34 |
| 60 | GLU | CA | −5.29 | 46.67 | −4.26 | 13.30 |
| 60 | GLU | CB | −4.10 | 45.94 | −4.91 | 13.77 |
| 60 | GLU | CG | −2.73 | 46.19 | −4.30 | 14.47 |
| 60 | GLU | CD | −2.37 | 47.65 | −4.23 | 14.81 |
| 60 | GLU | OE1 | −2.75 | 48.41 | −5.16 | 15.14 |
| 60 | GLU | OE2 | −1.69 | 48.04 | −3.25 | 15.19 |
| 60 | GLU | C | −6.54 | 46.02 | −4.82 | 13.01 |
| 60 | GLU | O | −6.73 | 45.99 | −6.04 | 12.97 |
| 61 | ASN | N | −7.39 | 45.50 | −3.95 | 12.60 |
| 61 | ASN | CA | −8.59 | 44.80 | −4.39 | 12.28 |
| 61 | ASN | CB | −9.54 | 45.71 | −5.17 | 12.26 |
| 61 | ASN | CG | −10.89 | 45.06 | −5.43 | 12.34 |
| 61 | ASN | OD1 | −11.35 | 44.22 | −4.65 | 12.33 |
| 61 | ASN | ND2 | −11.54 | 45.46 | −6.53 | 12.36 |
| 61 | ASN | C | −8.14 | 43.64 | −5.27 | 12.04 |
| 61 | ASN | O | −8.72 | 43.37 | −6.32 | 11.90 |
| 62 | PHE | N | −7.08 | 42.97 | −4.82 | 11.76 |
| 62 | PHE | CA | −6.51 | 41.81 | −5.50 | 11.47 |
| 62 | PHE | CB | −5.15 | 41.46 | −4.89 | 11.52 |
| 62 | PHE | CG | −4.47 | 40.29 | −5.53 | 11.77 |
| 62 | PHE | CD1 | −3.81 | 40.43 | −6.75 | 11.88 |
| 62 | PHE | CD2 | −4.47 | 39.04 | −4.91 | 11.85 |
| 62 | PHE | CE1 | −3.15 | 39.35 | −7.33 | 11.91 |
| 62 | PHE | CE2 | −3.82 | 37.95 | −5.50 | 11.88 |
| 62 | PHE | CZ | −3.16 | 38.10 | −6.71 | 11.91 |
| 62 | PHE | C | −7.48 | 40.66 | −5.26 | 11.24 |
| 62 | PHE | O | −7.98 | 40.50 | −4.14 | 11.11 |
| 63 | PRO | N | −7.76 | 39.86 | −6.29 | 11.08 |
| 63 | PRO | CD | −7.35 | 40.06 | −7.70 | 10.99 |
| 63 | PRO | CA | −8.68 | 38.72 | −6.18 | 10.84 |
| 63 | PRO | CB | −9.06 | 38.46 | −7.64 | 10.94 |
| 63 | PRO | CG | −7.77 | 38.76 | −8.35 | 11.00 |
| 63 | PRO | C | −8.03 | 37.50 | −5.53 | 10.71 |
| 63 | PRO | O | −7.88 | 36.45 | −6.15 | 10.70 |
| 64 | GLY | N | −7.65 | 37.65 | −4.26 | 10.53 |
| 64 | GLY | CA | −7.01 | 36.56 | −3.55 | 10.28 |
| 64 | GLY | C | −6.47 | 37.03 | −2.22 | 10.11 |
| 64 | GLY | O | −6.09 | 38.19 | −2.08 | 10.11 |
| 65 | GLN | N | −6.43 | 36.14 | −1.23 | 9.93 |
| 65 | GLN | CA | −5.92 | 36.49 | 0.09 | 9.84 |
| 65 | GLN | CB | −6.17 | 35.34 | 1.07 | 9.86 |
| 65 | GLN | CG | −7.64 | 35.04 | 1.34 | 10.07 |
| 65 | GLN | CD | −8.30 | 36.09 | 2.21 | 10.18 |
| 65 | GLN | OE1 | −7.66 | 36.67 | 3.10 | 10.15 |
| 65 | GLN | NE2 | −9.59 | 36.34 | 1.96 | 10.31 |
| 65 | GLN | C | −4.42 | 36.78 | 0.04 | 9.68 |
| 65 | GLN | O | −3.94 | 37.73 | 0.67 | 9.61 |
| 66 | THR | N | −3.71 | 35.94 | −0.70 | 9.64 |
| 66 | THR | CA | −2.26 | 36.02 | −0.86 | 9.57 |
| 66 | THR | CB | −1.57 | 34.85 | −0.14 | 9.54 |
| 66 | THR | OG1 | −1.96 | 33.61 | −0.76 | 9.57 |
| 66 | THR | CG2 | −1.97 | 34.80 | 1.33 | 9.59 |
| 66 | THR | C | −1.88 | 35.90 | −2.33 | 9.54 |
| 66 | THR | O | −2.74 | 35.66 | −3.18 | 9.47 |
| 67 | CYS | N | −0.59 | 36.06 | −2.62 | 9.58 |
| 67 | CYS | CA | −0.09 | 35.93 | −3.98 | 9.69 |
| 67 | CYS | CB | 0.86 | 37.07 | −4.33 | 10.27 |
| 67 | CYS | SG | 0.04 | 38.68 | −4.41 | 10.95 |
| 67 | CYS | C | 0.66 | 34.60 | −4.01 | 9.38 |
| 67 | CYS | O | 1.51 | 34.34 | −3.17 | 9.38 |
| 68 | ILE | N | 0.31 | 33.76 | −4.98 | 9.24 |
| 68 | ILE | CA | 0.94 | 32.45 | −5.12 | 9.20 |
| 68 | ILE | CB | −0.05 | 31.33 | −4.78 | 9.22 |
| 68 | ILE | CG2 | 0.65 | 29.97 | −4.90 | 9.09 |
| 68 | ILE | CG1 | −0.59 | 31.54 | −3.36 | 9.16 |
| 68 | ILE | CD1 | −1.65 | 30.53 | −2.94 | 9.32 |
| 68 | ILE | C | 1.34 | 32.34 | −6.58 | 9.23 |
| 68 | ILE | O | 0.48 | 32.24 | −7.46 | 9.15 |
| 69 | SER | N | 2.64 | 32.35 | −6.83 | 9.40 |
| 69 | SER | CA | 3.17 | 32.30 | −8.18 | 9.44 |
| 69 | SER | CB | 4.12 | 33.48 | −8.38 | 9.50 |
| 69 | SER | OG | 3.54 | 34.67 | −7.84 | 9.31 |
| 69 | SER | C | 3.87 | 30.98 | −8.47 | 9.48 |
| 69 | SER | O | 4.70 | 30.54 | −7.67 | 9.68 |
| 70 | VAL | N | 3.54 | 30.35 | −9.59 | 9.51 |
| 70 | VAL | CA | 4.16 | 29.06 | −9.90 | 9.65 |
| 70 | VAL | CB | 3.10 | 27.93 | −9.93 | 9.70 |
| 70 | VAL | CG1 | 3.79 | 26.59 | −10.19 | 9.57 |
| 70 | VAL | CG2 | 2.35 | 27.89 | −8.61 | 9.59 |
| 70 | VAL | C | 4.93 | 29.04 | −11.22 | 9.79 |
| 70 | VAL | O | 4.47 | 29.56 | −12.23 | 9.89 |
| 71 | ASN | N | 6.12 | 28.45 | −11.18 | 10.00 |
| 71 | ASN | CA | 7.00 | 28.30 | −12.35 | 10.15 |
| 71 | ASN | CB | 6.50 | 27.14 | −13.21 | 10.16 |
| 71 | ASN | CG | 6.49 | 25.83 | −12.46 | 10.32 |
| 71 | ASN | OD1 | 7.40 | 25.55 | −11.67 | 10.33 |
| 71 | ASN | ND2 | 5.48 | 25.00 | −12.71 | 10.38 |
| 71 | ASN | C | 7.25 | 29.54 | −13.21 | 10.18 |
| 71 | ASN | O | 8.11 | 30.35 | −12.89 | 10.13 |
| 72 | GLU | N | 6.50 | 29.68 | −14.31 | 10.29 |
| 72 | GLU | CA | 6.70 | 30.83 | −15.18 | 10.24 |
| 72 | GLU | CB | 5.90 | 30.66 | −16.49 | 10.29 |
| 72 | GLU | CG | 4.40 | 30.90 | −16.41 | 10.47 |
| 72 | GLU | CD | 3.63 | 29.76 | −15.78 | 10.52 |
| 72 | GLU | OE1 | 4.22 | 28.69 | −15.52 | 10.61 |
| 72 | GLU | OE2 | 2.41 | 29.92 | −15.55 | 10.59 |
| 72 | GLU | C | 6.33 | 32.14 | −14.48 | 10.16 |
| 72 | GLU | O | 6.86 | 33.19 | −14.83 | 10.23 |
| 73 | GLU | N | 5.45 | 32.07 | −13.48 | 10.17 |
| 73 | GLU | CA | 5.07 | 33.27 | −12.74 | 10.16 |
| 73 | GLU | CB | 3.70 | 33.09 | −12.10 | 10.39 |
| 73 | GLU | CG | 2.64 | 32.63 | −13.07 | 10.62 |
| 73 | GLU | CD | 1.36 | 32.28 | −12.37 | 10.77 |
| 73 | GLU | OE1 | 1.43 | 31.66 | −11.28 | 10.88 |
| 73 | GLU | OE2 | 0.28 | 32.62 | −12.91 | 10.93 |
| 73 | GLU | C | 6.08 | 33.52 | −11.63 | 10.13 |
| 73 | GLU | O | 6.46 | 32.61 | −10.90 | 10.04 |
| 74 | VAL | N | 6.52 | 34.77 | −11.52 | 10.35 |
| 74 | VAL | CA | 7.50 | 35.19 | −10.53 | 10.46 |
| 74 | VAL | CB | 8.49 | 36.20 | −11.14 | 10.53 |
| 74 | VAL | CG1 | 9.49 | 36.69 | −10.08 | 10.53 |
| 74 | VAL | CG2 | 9.23 | 35.56 | −12.31 | 10.54 |
| 74 | VAL | C | 6.86 | 35.85 | −9.31 | 10.56 |
| 74 | VAL | O | 7.29 | 35.66 | −8.17 | 10.57 |
| 75 | ALA | N | 5.83 | 36.64 | −9.57 | 10.70 |
| 75 | ALA | CA | 5.14 | 37.34 | −8.49 | 10.79 |
| 75 | ALA | CB | 5.96 | 38.56 | −8.08 | 10.84 |
| 75 | ALA | C | 3.74 | 37.77 | −8.90 | 10.84 |
| 75 | ALA | O | 3.43 | 37.83 | −10.08 | 10.81 |
| 76 | HIS | N | 2.92 | 38.05 | −7.90 | 10.94 |
| 76 | HIS | CA | 1.54 | 38.48 | −8.11 | 11.16 |
| 76 | HIS | CB | 1.53 | 39.77 | −8.95 | 11.62 |
| 76 | HIS | CG | 2.11 | 40.97 | −8.26 | 12.06 |
| 76 | HIS | CD2 | 1.62 | 42.22 | −8.06 | 12.29 |
| 76 | HIS | ND1 | 3.36 | 40.96 | −7.66 | 12.32 |
| 76 | HIS | CE1 | 3.60 | 42.14 | −7.13 | 12.37 |
| 76 | HIS | NE2 | 2.57 | 42.93 | −7.36 | 12.45 |
| 76 | HIS | C | 0.64 | 37.42 | −8.74 | 11.03 |
| 76 | HIS | O | −0.40 | 37.75 | −9.33 | 11.11 |
| 77 | GLY | N | 1.00 | 36.15 | −8.62 | 10.90 |
| 77 | GLY | CA | 0.16 | 35.09 | −9.18 | 10.76 |
| 77 | GLY | C | −1.19 | 35.06 | −8.46 | 10.74 |
| 77 | GLY | O | −1.25 | 35.25 | −7.24 | 10.61 |
| 78 | ILE | N | −2.27 | 34.83 | −9.21 | 10.69 |
| 78 | ILE | CA | −3.62 | 34.79 | −8.63 | 10.77 |
| 78 | ILE | CB | −4.67 | 35.36 | −9.63 | 10.80 |
| 78 | ILE | CG2 | −6.06 | 35.29 | −9.01 | 10.82 |
| 78 | ILE | CG1 | −4.33 | 36.81 | −10.00 | 10.90 |
| 78 | ILE | CD1 | −5.20 | 37.39 | −11.13 | 11.05 |
| 78 | ILE | C | −4.03 | 33.36 | −8.26 | 10.69 |
| 78 | ILE | O | −4.05 | 32.49 | −9.12 | 10.75 |
| 79 | PRO | N | −4.34 | 33.10 | −6.97 | 10.64 |
| 79 | PRO | CD | −4.24 | 34.00 | −5.81 | 10.79 |
| 79 | PRO | CA | −4.75 | 31.76 | −6.55 | 10.78 |
| 79 | PRO | CB | −5.12 | 31.95 | −5.08 | 10.74 |
| 79 | PRO | CG | −4.16 | 33.03 | −4.65 | 10.72 |
| 79 | PRO | C | −5.94 | 31.32 | −7.40 | 10.74 |
| 79 | PRO | O | −6.85 | 32.12 | −7.66 | 10.77 |
| 80 | SER | N | −5.94 | 30.07 | −7.82 | 10.76 |
| 80 | SER | CA | −6.99 | 29.57 | −8.70 | 10.89 |
| 80 | SER | CB | −6.71 | 30.02 | −10.12 | 10.91 |
| 80 | SER | OG | −5.49 | 29.44 | −10.57 | 10.83 |
| 80 | SER | C | −7.09 | 28.05 | −8.68 | 10.97 |
| 80 | SER | O | −6.63 | 27.39 | −7.75 | 10.96 |
| 81 | LYS | N | −7.68 | 27.50 | −9.74 | 11.05 |
| 81 | LYS | CA | −7.85 | 26.06 | −9.87 | 11.11 |
| 81 | LYS | CB | −8.93 | 25.77 | −10.91 | 11.30 |
| 81 | LYS | CG | −10.29 | 26.34 | −10.54 | 11.69 |
| 81 | LYS | CD | −11.26 | 26.34 | −11.71 | 12.04 |
| 81 | LYS | CE | −12.62 | 26.91 | −11.30 | 12.20 |
| 81 | LYS | NZ | −13.55 | 27.00 | −12.46 | 12.37 |
| 81 | LYS | C | −6.54 | 25.38 | −10.26 | 10.98 |
| 81 | LYS | O | −6.47 | 24.15 | −10.31 | 10.91 |
| 82 | ARG | N | −5.51 | 26.18 | −10.55 | 10.88 |
| 82 | ARG | CA | −4.21 | 25.63 | −10.93 | 10.79 |
| 82 | ARG | CB | −3.16 | 26.74 | −11.07 | 10.81 |
| 82 | ARG | CG | −1.79 | 26.21 | −11.43 | 10.88 |
| 82 | ARG | CD | −0.82 | 27.33 | −11.77 | 11.00 |
| 82 | ARG | NE | 0.41 | 26.79 | −12.36 | 11.17 |
| 82 | ARG | CZ | 1.25 | 27.50 | −13.11 | 11.12 |
| 82 | ARG | NH1 | 1.00 | 28.77 | −13.37 | 11.26 |
| 82 | ARG | NH2 | 2.35 | 26.93 | −13.61 | 11.15 |
| 82 | ARG | C | −3.74 | 24.62 | −9.89 | 10.73 |
| 82 | ARG | O | −3.60 | 24.94 | −8.72 | 10.61 |
| 83 | VAL | N | −3.48 | 23.39 | −10.34 | 10.71 |
| 83 | VAL | CA | −3.03 | 22.35 | −9.43 | 10.66 |
| 83 | VAL | CB | −3.58 | 20.97 | −9.86 | 10.88 |
| 83 | VAL | CG1 | −3.00 | 20.58 | −11.20 | 11.18 |
| 83 | VAL | CG2 | −3.24 | 19.93 | −8.81 | 11.07 |
| 83 | VAL | C | −1.50 | 22.28 | −9.33 | 10.53 |
| 83 | VAL | O | −0.80 | 22.34 | −10.34 | 10.33 |
| 84 | ILE | N | −1.01 | 22.16 | −8.10 | 10.44 |
| 84 | ILE | CA | 0.43 | 22.05 | −7.86 | 10.41 |
| 84 | ILE | CB | 0.78 | 22.17 | −6.36 | 10.49 |
| 84 | ILE | CG2 | 2.24 | 21.79 | −6.14 | 10.39 |
| 84 | ILE | CG1 | 0.48 | 23.59 | −5.87 | 10.59 |
| 84 | ILE | CD1 | 1.13 | 24.69 | −6.68 | 10.80 |
| 84 | ILE | C | 0.89 | 20.67 | −8.32 | 10.41 |
| 84 | ILE | O | 0.20 | 19.68 | −8.10 | 10.33 |
| 85 | ARG | N | 2.05 | 20.62 | −8.96 | 10.45 |
| 85 | ARG | CA | 2.62 | 19.37 | −9.46 | 10.65 |
| 85 | ARG | CB | 2.66 | 19.39 | −10.99 | 11.00 |
| 85 | ARG | CG | 1.30 | 19.54 | −11.64 | 11.42 |
| 85 | ARG | CD | 0.48 | 18.29 | −11.41 | 11.83 |
| 85 | ARG | NE | 1.04 | 17.16 | −12.15 | 12.31 |
| 85 | ARG | CZ | 0.60 | 16.75 | −13.34 | 12.40 |
| 85 | ARG | NH1 | −0.43 | 17.36 | −13.91 | 12.49 |
| 85 | ARG | NH2 | 1.19 | 15.73 | −13.95 | 12.62 |
| 85 | ARG | C | 4.03 | 19.16 | −8.93 | 10.67 |
| 85 | ARG | O | 4.71 | 20.11 | −8.54 | 10.63 |
| 86 | GLU | N | 4.46 | 17.91 | −8.90 | 10.68 |
| 86 | GLU | CA | 5.80 | 17.55 | −8.43 | 10.76 |
| 86 | GLU | CB | 5.99 | 16.03 | −8.60 | 11.03 |
| 86 | GLU | CG | 7.34 | 15.46 | −8.16 | 11.44 |
| 86 | GLU | CD | 7.51 | 15.40 | −6.67 | 11.57 |
| 86 | GLU | OE1 | 6.50 | 15.46 | −5.93 | 11.74 |
| 86 | GLU | OE2 | 8.68 | 15.28 | −6.21 | 11.75 |
| 86 | GLU | C | 6.84 | 18.32 | −9.25 | 10.57 |
| 86 | GLU | O | 6.76 | 18.36 | −10.48 | 10.62 |
| 87 | GLY | N | 7.81 | 18.93 | −8.57 | 10.32 |
| 87 | GLY | CA | 8.84 | 19.67 | −9.27 | 9.84 |
| 87 | GLY | C | 8.54 | 21.15 | −9.50 | 9.48 |
| 87 | GLY | O | 9.38 | 21.88 | −10.04 | 9.46 |
| 88 | ASP | N | 7.35 | 21.59 | −9.11 | 9.17 |
| 88 | ASP | CA | 6.97 | 23.00 | −9.29 | 8.91 |
| 88 | ASP | CB | 5.49 | 23.23 | −8.93 | 9.17 |
| 88 | ASP | CG | 4.53 | 22.89 | −10.06 | 9.36 |
| 88 | ASP | OD1 | 4.97 | 22.61 | −11.19 | 9.56 |
| 88 | ASP | OD2 | 3.30 | 22.92 | −9.80 | 9.35 |
| 88 | ASP | C | 7.80 | 23.92 | −8.39 | 8.57 |
| 88 | ASP | O | 8.13 | 23.57 | −7.26 | 8.51 |
| 89 | LEU | N | 8.13 | 25.10 | −8.91 | 8.39 |
| 89 | LEU | CA | 8.86 | 26.11 | −8.14 | 8.03 |
| 89 | LEU | CB | 9.90 | 26.82 | −9.02 | 8.06 |
| 89 | LEU | CG | 10.69 | 27.97 | −8.37 | 8.15 |
| 89 | LEU | CD1 | 11.29 | 27.55 | −7.04 | 8.16 |
| 89 | LEU | CD2 | 11.80 | 28.41 | −9.34 | 8.15 |
| 89 | LEU | C | 7.75 | 27.08 | −7.74 | 7.81 |
| 89 | LEU | O | 7.15 | 27.75 | −8.58 | 7.78 |
| 90 | VAL | N | 7.48 | 27.14 | −6.44 | 7.61 |
| 90 | VAL | CA | 6.40 | 27.97 | −5.94 | 7.45 |
| 90 | VAL | CB | 5.36 | 27.10 | −5.19 | 7.48 |
| 90 | VAL | CG1 | 4.15 | 27.93 | −4.80 | 7.64 |
| 90 | VAL | CG2 | 4.95 | 25.92 | −6.06 | 7.67 |
| 90 | VAL | C | 6.80 | 29.10 | −5.00 | 7.23 |
| 90 | VAL | O | 7.65 | 28.93 | −4.13 | 7.21 |
| 91 | ASN | N | 6.21 | 30.27 | −5.22 | 7.18 |
| 91 | ASN | CA | 6.44 | 31.40 | −4.33 | 7.02 |
| 91 | ASN | CB | 6.99 | 32.63 | −5.05 | 7.35 |
| 91 | ASN | CG | 7.15 | 33.81 | −4.10 | 7.73 |
| 91 | ASN | OD1 | 6.29 | 34.70 | −4.03 | 8.01 |
| 91 | ASN | ND2 | 8.24 | 33.82 | −3.34 | 7.95 |
| 91 | ASN | C | 5.12 | 31.77 | −3.69 | 6.84 |
| 91 | ASN | O | 4.09 | 31.94 | −4.36 | 6.56 |
| 92 | ILE | N | 5.16 | 31.88 | −2.38 | 6.72 |
| 92 | ILE | CA | 3.99 | 32.26 | −1.62 | 6.72 |
| 92 | ILE | CB | 3.65 | 31.19 | −0.56 | 6.92 |
| 92 | ILE | CG2 | 2.50 | 31.67 | 0.32 | 7.04 |
| 92 | ILE | CG1 | 3.30 | 29.87 | −1.26 | 7.31 |
| 92 | ILE | CD1 | 3.10 | 28.69 | −0.33 | 7.69 |
| 92 | ILE | C | 4.39 | 33.56 | −0.95 | 6.57 |
| 92 | ILE | O | 5.38 | 33.61 | −0.22 | 6.50 |
| 93 | ASP | N | 3.64 | 34.62 | −1.23 | 6.66 |
| 93 | ASP | CA | 3.93 | 35.92 | −0.67 | 6.75 |
| 93 | ASP | CB | 4.21 | 36.93 | −1.78 | 7.16 |
| 93 | ASP | CG | 4.79 | 38.23 | −1.25 | 7.51 |
| 93 | ASP | OD1 | 4.32 | 38.70 | −0.21 | 7.76 |
| 93 | ASP | OD2 | 5.71 | 38.78 | −1.89 | 7.87 |
| 93 | ASP | C | 2.72 | 36.35 | 0.15 | 6.68 |
| 93 | ASP | O | 1.63 | 36.56 | −0.38 | 6.56 |
| 94 | VAL | N | 2.94 | 36.46 | 1.46 | 6.81 |
| 94 | VAL | CA | 1.89 | 36.81 | 2.40 | 6.91 |
| 94 | VAL | CB | 1.88 | 35.78 | 3.55 | 6.97 |
| 94 | VAL | CG1 | 0.77 | 36.10 | 4.54 | 7.20 |
| 94 | VAL | CG2 | 1.70 | 34.37 | 2.98 | 7.24 |
| 94 | VAL | C | 2.06 | 38.21 | 2.98 | 6.83 |
| 94 | VAL | O | 3.11 | 38.52 | 3.55 | 6.83 |
| 95 | SER | N | 1.04 | 39.05 | 2.81 | 6.97 |
| 95 | SER | CA | 1.05 | 40.41 | 3.34 | 7.04 |
| 95 | SER | CB | 1.39 | 41.44 | 2.26 | 7.24 |
| 95 | SER | OG | 0.46 | 41.42 | 1.20 | 7.21 |
| 95 | SER | C | −0.32 | 40.67 | 3.94 | 7.05 |
| 95 | SER | O | −1.35 | 40.29 | 3.38 | 7.08 |
| 96 | ALA | N | −0.33 | 41.31 | 5.11 | 7.08 |
| 96 | ALA | CA | −1.56 | 41.60 | 5.82 | 7.05 |
| 96 | ALA | CB | −2.02 | 40.35 | 6.57 | 7.10 |
| 96 | ALA | C | −1.37 | 42.75 | 6.80 | 7.11 |
| 96 | ALA | O | −0.24 | 43.20 | 7.04 | 6.96 |
| 97 | LEU | N | −2.47 | 43.19 | 7.38 | 7.18 |
| 97 | LEU | CA | −2.46 | 44.27 | 8.36 | 7.37 |
| 97 | LEU | CB | −2.91 | 45.59 | 7.71 | 7.46 |
| 97 | LEU | CG | −4.33 | 45.69 | 7.14 | 7.59 |
| 97 | LEU | CD1 | −4.78 | 47.15 | 7.16 | 7.72 |
| 97 | LEU | CD2 | −4.38 | 45.14 | 5.71 | 7.62 |
| 97 | LEU | C | −3.39 | 43.94 | 9.52 | 7.47 |
| 97 | LEU | O | −4.37 | 43.21 | 9.34 | 7.49 |
| 98 | LYS | N | −3.06 | 44.44 | 10.70 | 7.66 |
| 98 | LYS | CA | −3.88 | 44.24 | 11.90 | 7.93 |
| 98 | LYS | CB | −3.43 | 43.01 | 12.70 | 7.99 |
| 98 | LYS | CG | −4.20 | 42.81 | 14.02 | 8.11 |
| 98 | LYS | CD | −3.86 | 41.48 | 14.69 | 8.29 |
| 98 | LYS | CE | −4.66 | 41.28 | 15.98 | 8.47 |
| 98 | LYS | NZ | −4.37 | 39.97 | 16.64 | 8.69 |
| 98 | LYS | C | −3.69 | 45.50 | 12.73 | 8.15 |
| 98 | LYS | O | −2.56 | 45.92 | 12.98 | 8.19 |
| 99 | ASN | N | −4.80 | 46.11 | 13.12 | 8.43 |
| 99 | ASN | CA | −4.78 | 47.34 | 13.90 | 8.51 |
| 99 | ASN | CB | −4.24 | 47.09 | 15.32 | 8.67 |
| 99 | ASN | CG | −5.12 | 46.13 | 16.12 | 8.73 |
| 99 | ASN | OD1 | −6.35 | 46.14 | 15.99 | 8.91 |
| 99 | ASN | ND2 | −4.50 | 45.30 | 16.96 | 8.82 |
| 99 | ASN | C | −3.96 | 48.45 | 13.22 | 8.56 |
| 99 | ASN | O | −3.28 | 49.23 | 13.89 | 8.66 |
| 100 | GLY | N | −4.03 | 48.49 | 11.89 | 8.45 |
| 100 | GLY | CA | −3.33 | 49.51 | 11.12 | 8.36 |
| 100 | GLY | C | −1.87 | 49.27 | 10.75 | 8.21 |
| 100 | GLY | O | −1.29 | 50.05 | 10.00 | 8.19 |
| 101 | TYR | N | −1.28 | 48.19 | 11.27 | 8.04 |
| 101 | TYR | CA | 0.12 | 47.86 | 11.01 | 7.75 |
| 101 | TYR | CB | 0.86 | 47.62 | 12.32 | 7.87 |
| 101 | TYR | CG | 0.96 | 48.88 | 13.15 | 8.16 |
| 101 | TYR | CD1 | 2.01 | 49.78 | 12.98 | 8.29 |
| 101 | TYR | CE1 | 2.05 | 50.99 | 13.68 | 8.58 |
| 101 | TYR | CD2 | −0.05 | 49.22 | 14.05 | 8.29 |
| 101 | TYR | CE2 | −0.02 | 50.43 | 14.75 | 8.54 |
| 101 | TYR | CZ | 1.03 | 51.31 | 14.55 | 8.61 |
| 101 | TYR | OH | 1.04 | 52.52 | 15.22 | 8.95 |
| 101 | TYR | C | 0.28 | 46.67 | 10.07 | 7.52 |
| 101 | TYR | O | −0.46 | 45.69 | 10.16 | 7.45 |
| 102 | TYR | N | 1.28 | 46.76 | 9.20 | 7.38 |
| 102 | TYR | CA | 1.55 | 45.74 | 8.18 | 7.22 |
| 102 | TYR | CB | 1.68 | 46.42 | 6.81 | 7.30 |
| 102 | TYR | CG | 0.41 | 47.03 | 6.23 | 7.57 |
| 102 | TYR | CD1 | −0.22 | 48.12 | 6.83 | 7.54 |
| 102 | TYR | CE1 | −1.35 | 48.72 | 6.25 | 7.75 |
| 102 | TYR | CD2 | −0.13 | 46.54 | 5.04 | 7.61 |
| 102 | TYR | CE2 | −1.25 | 47.12 | 4.46 | 7.68 |
| 102 | TYR | CZ | −1.86 | 48.20 | 5.07 | 7.86 |
| 102 | TYR | OH | −2.97 | 48.77 | 4.48 | 7.96 |
| 102 | TYR | C | 2.78 | 44.86 | 8.34 | 7.07 |
| 102 | TYR | O | 3.73 | 45.21 | 9.04 | 6.95 |
| 103 | ALA | N | 2.74 | 43.72 | 7.65 | 7.07 |
| 103 | ALA | CA | 3.84 | 42.76 | 7.57 | 6.90 |
| 103 | ALA | CB | 3.64 | 41.60 | 8.52 | 6.96 |
| 103 | ALA | C | 3.79 | 42.29 | 6.12 | 6.75 |
| 103 | ALA | O | 2.73 | 42.31 | 5.48 | 6.84 |
| 104 | ASP | N | 4.94 | 41.87 | 5.60 | 6.61 |
| 104 | ASP | CA | 5.06 | 41.42 | 4.22 | 6.57 |
| 104 | ASP | CB | 5.46 | 42.62 | 3.36 | 6.79 |
| 104 | ASP | CG | 5.39 | 42.35 | 1.87 | 6.84 |
| 104 | ASP | OD1 | 5.12 | 41.20 | 1.47 | 6.89 |
| 104 | ASP | OD2 | 5.61 | 43.31 | 1.10 | 6.72 |
| 104 | ASP | C | 6.18 | 40.38 | 4.24 | 6.47 |
| 104 | ASP | O | 7.30 | 40.66 | 4.67 | 6.38 |
| 105 | THR | N | 5.88 | 39.16 | 3.80 | 6.50 |
| 105 | THR | CA | 6.90 | 38.13 | 3.80 | 6.44 |
| 105 | THR | CB | 6.94 | 37.40 | 5.17 | 6.46 |
| 105 | THR | OG1 | 8.02 | 36.46 | 5.19 | 6.45 |
| 105 | THR | CG2 | 5.62 | 36.69 | 5.43 | 6.53 |
| 105 | THR | C | 6.62 | 37.14 | 2.67 | 6.46 |
| 105 | THR | O | 5.49 | 37.05 | 2.19 | 6.55 |
| 106 | GLY | N | 7.63 | 36.40 | 2.25 | 6.43 |
| 106 | GLY | CA | 7.42 | 35.46 | 1.17 | 6.51 |
| 106 | GLY | C | 8.54 | 34.46 | 1.06 | 6.59 |
| 106 | GLY | O | 9.64 | 34.67 | 1.56 | 6.44 |
| 107 | ILE | N | 8.25 | 33.36 | 0.38 | 6.68 |
| 107 | ILE | CA | 9.25 | 32.32 | 0.23 | 6.71 |
| 107 | ILE | CB | 9.26 | 31.42 | 1.49 | 6.73 |
| 107 | ILE | CG2 | 7.89 | 30.77 | 1.67 | 6.75 |
| 107 | ILE | CG1 | 10.36 | 30.37 | 1.37 | 7.04 |
| 107 | ILE | CD1 | 10.63 | 29.65 | 2.66 | 7.53 |
| 107 | ILE | C | 9.02 | 31.45 | −1.00 | 6.69 |
| 107 | ILE | O | 7.89 | 31.09 | −1.32 | 6.47 |
| 108 | SER | N | 10.11 | 31.14 | −1.69 | 6.84 |
| 108 | SER | CA | 10.04 | 30.27 | −2.86 | 7.04 |
| 108 | SER | CB | 10.87 | 30.83 | −4.02 | 7.01 |
| 108 | SER | OG | 10.27 | 31.98 | −4.58 | 7.00 |
| 108 | SER | C | 10.64 | 28.94 | −2.42 | 7.18 |
| 108 | SER | O | 11.60 | 28.91 | −1.65 | 7.03 |
| 109 | PHE | N | 10.07 | 27.86 | −2.93 | 7.45 |
| 109 | PHE | CA | 10.54 | 26.52 | −2.62 | 7.76 |
| 109 | PHE | CB | 9.95 | 26.02 | −1.29 | 7.72 |
| 109 | PHE | CG | 8.45 | 26.09 | −1.23 | 7.67 |
| 109 | PHE | CD1 | 7.81 | 27.29 | −0.92 | 7.72 |
| 109 | PHE | CD2 | 7.67 | 24.97 | −1.50 | 7.74 |
| 109 | PHE | CE1 | 6.41 | 27.38 | −0.88 | 7.73 |
| 109 | PHE | CE2 | 6.27 | 25.04 | −1.46 | 7.72 |
| 109 | PHE | CZ | 5.64 | 26.25 | −1.15 | 7.66 |
| 109 | PHE | C | 10.15 | 25.59 | −3.75 | 8.02 |
| 109 | PHE | O | 9.33 | 25.94 | −4.59 | 7.97 |
| 110 | VAL | N | 10.77 | 24.41 | −3.78 | 8.40 |
| 110 | VAL | CA | 10.48 | 23.41 | −4.79 | 8.73 |
| 110 | VAL | CB | 11.78 | 22.72 | −5.26 | 8.76 |
| 110 | VAL | CG1 | 11.46 | 21.58 | −6.22 | 8.69 |
| 110 | VAL | CG2 | 12.69 | 23.74 | −5.94 | 8.75 |
| 110 | VAL | C | 9.56 | 22.37 | −4.17 | 9.11 |
| 110 | VAL | O | 9.80 | 21.93 | −3.04 | 9.12 |
| 111 | VAL | N | 8.51 | 22.00 | −4.89 | 9.46 |
| 111 | VAL | CA | 7.57 | 21.00 | −4.42 | 9.96 |
| 111 | VAL | CB | 6.20 | 21.16 | −5.11 | 9.94 |
| 111 | VAL | CG1 | 5.24 | 20.07 | −4.63 | 9.81 |
| 111 | VAL | CG2 | 5.63 | 22.53 | −4.80 | 9.87 |
| 111 | VAL | C | 8.16 | 19.64 | −4.77 | 10.35 |
| 111 | VAL | O | 8.31 | 19.31 | −5.94 | 10.37 |
| 112 | GLY | N | 8.52 | 18.88 | −3.74 | 10.81 |
| 112 | GLY | CA | 9.11 | 17.58 | −3.98 | 11.24 |
| 112 | GLY | C | 10.44 | 17.73 | −4.69 | 11.57 |
| 112 | GLY | O | 11.32 | 18.46 | −4.24 | 11.67 |
| 113 | GLU | N | 10.59 | 17.03 | −5.82 | 11.85 |
| 113 | GLU | CA | 11.82 | 17.09 | −6.58 | 12.17 |
| 113 | GLU | CB | 12.58 | 15.77 | −6.45 | 12.54 |
| 113 | GLU | CG | 12.85 | 15.33 | −5.02 | 13.07 |
| 113 | GLU | CD | 13.30 | 13.89 | −4.97 | 13.36 |
| 113 | GLU | OE1 | 12.56 | 13.03 | −5.50 | 13.72 |
| 113 | GLU | OE2 | 14.38 | 13.61 | −4.42 | 13.60 |
| 113 | GLU | C | 11.59 | 17.38 | −8.06 | 12.22 |
| 113 | GLU | O | 10.62 | 16.91 | −8.66 | 12.19 |
| 114 | SER | N | 12.51 | 18.14 | −8.64 | 12.26 |
| 114 | SER | CA | 12.49 | 18.50 | −10.05 | 12.29 |
| 114 | SER | CB | 12.64 | 20.02 | −10.21 | 12.45 |
| 114 | SER | OG | 12.82 | 20.38 | −11.56 | 12.71 |
| 114 | SER | C | 13.69 | 17.82 | −10.72 | 12.19 |
| 114 | SER | O | 14.65 | 17.47 | −10.04 | 12.16 |
| 115 | ASP | N | 13.62 | 17.60 | −12.04 | 12.18 |
| 115 | ASP | CA | 14.73 | 16.97 | −12.76 | 12.17 |
| 115 | ASP | CB | 14.28 | 16.48 | −14.13 | 12.77 |
| 115 | ASP | CG | 13.67 | 17.59 | −14.97 | 13.26 |
| 115 | ASP | OD1 | 12.73 | 18.24 | −14.47 | 13.73 |
| 115 | ASP | OD2 | 14.12 | 17.81 | −16.11 | 13.79 |
| 115 | ASP | C | 15.87 | 17.95 | −12.95 | 11.89 |
| 115 | ASP | O | 16.99 | 17.57 | −13.31 | 11.74 |
| 116 | ASP | N | 15.57 | 19.23 | −12.72 | 11.63 |
| 116 | ASP | CA | 16.56 | 20.29 | −12.88 | 11.41 |
| 116 | ASP | CB | 16.02 | 21.40 | −13.78 | 11.67 |
| 116 | ASP | CG | 17.11 | 22.33 | −14.27 | 11.80 |
| 116 | ASP | OD1 | 18.14 | 22.47 | −13.57 | 11.94 |
| 116 | ASP | OD2 | 16.93 | 22.93 | −15.35 | 11.91 |
| 116 | ASP | C | 16.91 | 20.87 | −11.52 | 11.19 |
| 116 | ASP | O | 16.08 | 21.53 | −10.89 | 11.12 |
| 117 | PRO | N | 18.14 | 20.65 | −11.04 | 11.03 |
| 117 | PRO | CD | 19.23 | 19.91 | −11.71 | 11.03 |
| 117 | PRO | CA | 18.57 | 21.16 | −9.75 | 10.91 |
| 117 | PRO | CB | 19.92 | 20.46 | −9.54 | 10.95 |
| 117 | PRO | CG | 20.44 | 20.34 | −10.93 | 11.01 |
| 117 | PRO | C | 18.68 | 22.69 | −9.68 | 10.85 |
| 117 | PRO | O | 18.85 | 23.26 | −8.60 | 10.70 |
| 118 | MET | N | 18.56 | 23.36 | −10.82 | 10.86 |
| 118 | MET | CA | 18.64 | 24.81 | −10.85 | 10.83 |
| 118 | MET | CB | 18.60 | 25.33 | −12.29 | 11.18 |
| 118 | MET | CG | 18.58 | 26.86 | −12.42 | 11.64 |
| 118 | MET | SD | 20.07 | 27.66 | −11.76 | 12.14 |
| 118 | MET | CE | 21.19 | 27.45 | −13.13 | 12.08 |
| 118 | MET | C | 17.49 | 25.43 | −10.05 | 10.57 |
| 118 | MET | O | 17.62 | 26.53 | −9.52 | 10.48 |
| 119 | LYS | N | 16.35 | 24.74 | −9.99 | 10.28 |
| 119 | LYS | CA | 15.23 | 25.28 | −9.23 | 10.14 |
| 119 | LYS | CB | 13.99 | 24.38 | −9.36 | 10.21 |
| 119 | LYS | CG | 13.34 | 24.45 | −10.74 | 10.29 |
| 119 | LYS | CD | 11.99 | 23.73 | −10.78 | 10.35 |
| 119 | LYS | CE | 11.26 | 24.00 | −12.10 | 10.55 |
| 119 | LYS | NZ | 9.99 | 23.23 | −12.22 | 10.63 |
| 119 | LYS | C | 15.63 | 25.44 | −7.76 | 9.96 |
| 119 | LYS | O | 15.46 | 26.51 | −7.19 | 9.99 |
| 120 | GLN | N | 16.19 | 24.39 | −7.17 | 9.82 |
| 120 | GLN | CA | 16.63 | 24.47 | −5.79 | 9.62 |
| 120 | GLN | CB | 17.09 | 23.09 | −5.29 | 9.62 |
| 120 | GLN | CG | 17.45 | 23.06 | −3.79 | 9.62 |
| 120 | GLN | CD | 16.29 | 23.50 | −2.91 | 9.61 |
| 120 | GLN | OE1 | 15.16 | 23.04 | −3.08 | 9.62 |
| 120 | GLN | NE2 | 16.56 | 24.40 | −1.97 | 9.65 |
| 120 | GLN | C | 17.78 | 25.48 | −5.65 | 9.52 |
| 120 | GLN | O | 17.87 | 26.18 | −4.64 | 9.51 |
| 121 | LYS | N | 18.64 | 25.57 | −6.67 | 9.41 |
| 121 | LYS | CA | 19.77 | 26.50 | −6.61 | 9.37 |
| 121 | LYS | CB | 20.64 | 26.41 | −7.89 | 9.37 |
| 121 | LYS | CG | 21.89 | 27.30 | −7.82 | 9.37 |
| 121 | LYS | CD | 22.84 | 27.13 | −9.02 | 9.39 |
| 121 | LYS | CE | 24.02 | 28.10 | −8.92 | 9.51 |
| 121 | LYS | NZ | 25.07 | 27.90 | −9.96 | 9.57 |
| 121 | LYS | C | 19.33 | 27.93 | −6.41 | 9.29 |
| 121 | LYS | O | 19.83 | 28.62 | −5.52 | 9.51 |
| 122 | VAL | N | 18.38 | 28.41 | −7.22 | 9.33 |
| 122 | VAL | CA | 17.94 | 29.79 | −7.07 | 9.34 |
| 122 | VAL | CB | 16.96 | 30.21 | −8.19 | 9.47 |
| 122 | VAL | CG1 | 17.68 | 30.20 | −9.53 | 9.60 |
| 122 | VAL | CG2 | 15.74 | 29.30 | −8.23 | 9.54 |
| 122 | VAL | C | 17.31 | 30.00 | −5.69 | 9.17 |
| 122 | VAL | O | 17.39 | 31.09 | −5.14 | 9.09 |
| 123 | CYS | N | 16.70 | 28.95 | −5.13 | 9.21 |
| 123 | CYS | CA | 16.13 | 29.07 | −3.80 | 9.07 |
| 123 | CYS | CB | 15.26 | 27.86 | −3.46 | 9.11 |
| 123 | CYS | SG | 13.69 | 27.80 | −4.39 | 8.94 |
| 123 | CYS | C | 17.26 | 29.21 | −2.78 | 9.04 |
| 123 | CYS | O | 17.18 | 30.04 | −1.89 | 8.96 |
| 124 | ASP | N | 18.30 | 28.38 | −2.91 | 9.10 |
| 124 | ASP | CA | 19.42 | 28.46 | −1.98 | 9.25 |
| 124 | ASP | CB | 20.49 | 27.39 | −2.29 | 9.57 |
| 124 | ASP | CG | 19.98 | 25.96 | −2.12 | 9.96 |
| 124 | ASP | OD1 | 19.07 | 25.72 | −1.29 | 10.11 |
| 124 | ASP | OD2 | 20.52 | 25.07 | −2.81 | 10.21 |
| 124 | ASP | C | 20.10 | 29.82 | −2.05 | 9.25 |
| 124 | ASP | O | 20.46 | 30.39 | −1.01 | 9.16 |
| 125 | VAL | N | 20.28 | 30.36 | −3.25 | 9.27 |
| 125 | VAL | CA | 20.95 | 31.65 | −3.40 | 9.34 |
| 125 | VAL | CB | 21.45 | 31.82 | −4.87 | 9.27 |
| 125 | VAL | CG1 | 22.22 | 33.13 | −5.03 | 9.38 |
| 125 | VAL | CG2 | 22.37 | 30.65 | −5.22 | 9.33 |
| 125 | VAL | C | 20.07 | 32.82 | −2.96 | 9.33 |
| 125 | VAL | O | 20.57 | 33.84 | −2.49 | 9.32 |
| 126 | ALA | N | 18.76 | 32.67 | −3.11 | 9.40 |
| 126 | ALA | CA | 17.85 | 33.71 | −2.67 | 9.55 |
| 126 | ALA | CB | 16.40 | 33.34 | −3.02 | 9.53 |
| 126 | ALA | C | 18.01 | 33.85 | −1.16 | 9.65 |
| 126 | ALA | O | 18.02 | 34.96 | −0.62 | 9.60 |
| 127 | THR | N | 18.15 | 32.72 | −0.47 | 9.87 |
| 127 | THR | CA | 18.32 | 32.72 | 0.98 | 10.18 |
| 127 | THR | CB | 18.20 | 31.30 | 1.56 | 10.35 |
| 127 | THR | OG1 | 16.87 | 30.81 | 1.35 | 10.71 |
| 127 | THR | CG2 | 18.49 | 31.29 | 3.06 | 10.60 |
| 127 | THR | C | 19.67 | 33.33 | 1.36 | 10.23 |
| 127 | THR | O | 19.74 | 34.15 | 2.28 | 10.12 |
| 128 | MET | N | 20.73 | 32.94 | 0.67 | 10.48 |
| 128 | MET | CA | 22.05 | 33.49 | 0.96 | 10.71 |
| 128 | MET | CB | 23.11 | 32.83 | 0.09 | 11.37 |
| 128 | MET | CG | 23.45 | 31.42 | 0.48 | 12.33 |
| 128 | MET | SD | 24.82 | 30.84 | −0.52 | 13.45 |
| 128 | MET | CE | 24.03 | 30.57 | −2.04 | 13.45 |
| 128 | MET | C | 22.05 | 34.99 | 0.70 | 10.48 |
| 128 | MET | O | 22.59 | 35.76 | 1.50 | 10.30 |
| 129 | ALA | N | 21.43 | 35.40 | −0.40 | 10.37 |
| 129 | ALA | CA | 21.37 | 36.82 | −0.74 | 10.39 |
| 129 | ALA | CB | 20.64 | 37.02 | −2.05 | 10.48 |
| 129 | ALA | C | 20.69 | 37.60 | 0.38 | 10.35 |
| 129 | ALA | O | 21.14 | 38.68 | 0.76 | 10.42 |
| 130 | PHE | N | 19.60 | 37.05 | 0.91 | 10.29 |
| 130 | PHE | CA | 18.90 | 37.73 | 1.98 | 10.16 |
| 130 | PHE | CB | 17.62 | 36.98 | 2.36 | 10.04 |
| 130 | PHE | CG | 16.90 | 37.61 | 3.50 | 9.87 |
| 130 | PHE | CD1 | 16.11 | 38.74 | 3.31 | 9.82 |
| 130 | PHE | CD2 | 17.07 | 37.13 | 4.79 | 9.87 |
| 130 | PHE | CE1 | 15.51 | 39.39 | 4.38 | 9.72 |
| 130 | PHE | CE2 | 16.47 | 37.77 | 5.88 | 9.79 |
| 130 | PHE | CZ | 15.69 | 38.91 | 5.67 | 9.70 |
| 130 | PHE | C | 19.80 | 37.85 | 3.21 | 10.19 |
| 130 | PHE | O | 19.91 | 38.92 | 3.81 | 10.16 |
| 131 | GLU | N | 20.45 | 36.75 | 3.57 | 10.29 |
| 131 | GLU | CA | 21.35 | 36.72 | 4.73 | 10.39 |
| 131 | GLU | CB | 21.95 | 35.31 | 4.91 | 10.58 |
| 131 | GLU | CG | 20.92 | 34.20 | 5.14 | 11.12 |
| 131 | GLU | CD | 21.56 | 32.81 | 5.15 | 11.40 |
| 131 | GLU | OE1 | 22.64 | 32.65 | 4.54 | 11.61 |
| 131 | GLU | OE2 | 20.96 | 31.89 | 5.76 | 11.68 |
| 131 | GLU | C | 22.49 | 37.72 | 4.58 | 10.35 |
| 131 | GLU | O | 22.86 | 38.40 | 5.54 | 10.21 |
| 132 | ASN | N | 23.06 | 37.79 | 3.38 | 10.46 |
| 132 | ASN | CA | 24.16 | 38.71 | 3.15 | 10.68 |
| 132 | ASN | CB | 24.82 | 38.41 | 1.81 | 10.77 |
| 132 | ASN | CG | 25.50 | 37.07 | 1.79 | 10.94 |
| 132 | ASN | OD1 | 25.77 | 36.49 | 2.85 | 11.09 |
| 132 | ASN | ND2 | 25.80 | 36.56 | 0.60 | 11.00 |
| 132 | ASN | C | 23.70 | 40.16 | 3.23 | 10.90 |
| 132 | ASN | O | 24.45 | 41.03 | 3.68 | 10.77 |
| 133 | ALA | N | 22.47 | 40.41 | 2.80 | 11.27 |
| 133 | ALA | CA | 21.92 | 41.77 | 2.83 | 11.71 |
| 133 | ALA | CB | 20.65 | 41.84 | 2.00 | 11.66 |
| 133 | ALA | C | 21.63 | 42.20 | 4.26 | 12.01 |
| 133 | ALA | O | 22.00 | 43.30 | 4.67 | 12.17 |
| 134 | ILE | N | 20.98 | 41.34 | 5.04 | 12.39 |
| 134 | ILE | CA | 20.67 | 41.74 | 6.41 | 12.81 |
| 134 | ILE | CB | 19.72 | 40.75 | 7.08 | 12.92 |
| 134 | ILE | CG2 | 18.41 | 40.70 | 6.30 | 13.10 |
| 134 | ILE | CG1 | 20.36 | 39.37 | 7.16 | 13.08 |
| 134 | ILE | CD1 | 19.61 | 38.41 | 8.05 | 13.19 |
| 134 | ILE | C | 21.92 | 41.88 | 7.27 | 12.95 |
| 134 | ILE | O | 21.90 | 42.57 | 8.29 | 12.97 |
| 135 | ALA | N | 23.01 | 41.23 | 6.85 | 13.25 |
| 135 | ALA | CA | 24.27 | 41.27 | 7.59 | 13.54 |
| 135 | ALA | CB | 25.36 | 40.55 | 6.80 | 13.43 |
| 135 | ALA | C | 24.76 | 42.66 | 7.97 | 13.78 |
| 135 | ALA | O | 25.27 | 42.85 | 9.08 | 14.02 |
| 136 | LYS | N | 24.64 | 43.64 | 7.09 | 14.12 |
| 136 | LYS | CA | 25.10 | 44.98 | 7.46 | 14.38 |
| 136 | LYS | CB | 26.24 | 45.44 | 6.55 | 14.80 |
| 136 | LYS | CG | 27.59 | 44.79 | 6.83 | 15.14 |
| 136 | LYS | CD | 28.67 | 45.41 | 5.95 | 15.50 |
| 136 | LYS | CE | 29.92 | 44.56 | 5.89 | 15.73 |
| 136 | LYS | NZ | 30.80 | 45.03 | 4.76 | 16.03 |
| 136 | LYS | C | 24.00 | 46.03 | 7.52 | 14.44 |
| 136 | LYS | O | 24.26 | 47.23 | 7.36 | 14.57 |
| 137 | VAL | N | 22.77 | 45.59 | 7.75 | 14.41 |
| 137 | VAL | CA | 21.66 | 46.53 | 7.89 | 14.37 |
| 137 | VAL | CB | 20.30 | 45.82 | 7.73 | 14.47 |
| 137 | VAL | CG1 | 19.18 | 46.73 | 8.20 | 14.47 |
| 137 | VAL | CG2 | 20.09 | 45.43 | 6.27 | 14.42 |
| 137 | VAL | C | 21.78 | 47.11 | 9.30 | 14.30 |
| 137 | VAL | O | 21.57 | 46.40 | 10.29 | 14.32 |
| 138 | LYS | N | 22.16 | 48.38 | 9.38 | 14.21 |
| 138 | LYS | CA | 22.31 | 49.08 | 10.65 | 14.10 |
| 138 | LYS | CB | 23.73 | 48.88 | 11.21 | 14.25 |
| 138 | LYS | CG | 24.07 | 47.42 | 11.56 | 14.53 |
| 138 | LYS | CD | 25.23 | 47.32 | 12.56 | 14.70 |
| 138 | LYS | CE | 24.83 | 47.88 | 13.94 | 14.84 |
| 138 | LYS | NZ | 23.62 | 47.17 | 14.50 | 15.00 |
| 138 | LYS | C | 22.04 | 50.57 | 10.45 | 13.84 |
| 138 | LYS | O | 22.07 | 51.06 | 9.32 | 13.78 |
| 139 | PRO | N | 21.76 | 51.31 | 11.54 | 13.62 |
| 139 | PRO | CD | 21.53 | 50.85 | 12.92 | 13.62 |
| 139 | PRO | CA | 21.49 | 52.75 | 11.42 | 13.44 |
| 139 | PRO | CB | 21.24 | 53.17 | 12.87 | 13.48 |
| 139 | PRO | CG | 20.66 | 51.96 | 13.48 | 13.54 |
| 139 | PRO | C | 22.69 | 53.48 | 10.82 | 13.30 |
| 139 | PRO | O | 23.83 | 53.21 | 11.19 | 13.29 |
| 140 | GLY | N | 22.42 | 54.40 | 9.89 | 13.05 |
| 140 | GLY | CA | 23.49 | 55.17 | 9.28 | 12.85 |
| 140 | GLY | C | 24.13 | 54.55 | 8.05 | 12.75 |
| 140 | GLY | O | 24.90 | 55.22 | 7.36 | 12.72 |
| 141 | THR | N | 23.83 | 53.28 | 7.77 | 12.65 |
| 141 | THR | CA | 24.39 | 52.61 | 6.61 | 12.58 |
| 141 | THR | CB | 24.45 | 51.07 | 6.81 | 12.80 |
| 141 | THR | OG1 | 23.11 | 50.54 | 6.86 | 13.18 |
| 141 | THR | CG2 | 25.16 | 50.74 | 8.11 | 13.07 |
| 141 | THR | C | 23.58 | 52.92 | 5.36 | 12.27 |
| 141 | THR | O | 22.39 | 53.24 | 5.44 | 12.24 |
| 142 | LYS | N | 24.22 | 52.81 | 4.21 | 12.00 |
| 142 | LYS | CA | 23.56 | 53.10 | 2.94 | 11.76 |
| 142 | LYS | CB | 24.61 | 53.24 | 1.83 | 11.96 |
| 142 | LYS | CG | 25.55 | 54.42 | 2.04 | 12.16 |
| 142 | LYS | CD | 26.70 | 54.38 | 1.05 | 12.55 |
| 142 | LYS | CE | 27.64 | 55.56 | 1.22 | 12.85 |
| 142 | LYS | NZ | 28.77 | 55.47 | 0.26 | 13.24 |
| 142 | LYS | C | 22.55 | 52.02 | 2.56 | 11.54 |
| 142 | LYS | O | 22.79 | 50.83 | 2.73 | 11.48 |
| 143 | LEU | N | 21.41 | 52.49 | 2.07 | 11.36 |
| 143 | LEU | CA | 20.34 | 51.62 | 1.63 | 11.24 |
| 143 | LEU | CB | 19.16 | 52.47 | 1.15 | 11.33 |
| 143 | LEU | CG | 18.00 | 51.73 | 0.49 | 11.35 |
| 143 | LEU | CD1 | 17.25 | 50.94 | 1.56 | 11.40 |
| 143 | LEU | CD2 | 17.05 | 52.73 | −0.18 | 11.36 |
| 143 | LEU | C | 20.86 | 50.76 | 0.48 | 11.12 |
| 143 | LEU | O | 20.55 | 49.57 | 0.39 | 11.16 |
| 144 | SER | N | 21.68 | 51.38 | −0.36 | 10.97 |
| 144 | SER | CA | 22.25 | 50.70 | −1.52 | 10.92 |
| 144 | SER | CB | 23.07 | 51.70 | −2.36 | 11.18 |
| 144 | SER | OG | 24.21 | 52.16 | −1.66 | 11.42 |
| 144 | SER | C | 23.11 | 49.48 | −1.18 | 10.76 |
| 144 | SER | O | 23.39 | 48.67 | −2.06 | 10.67 |
| 145 | ASN | N | 23.54 | 49.36 | 0.08 | 10.61 |
| 145 | ASN | CA | 24.34 | 48.20 | 0.47 | 10.40 |
| 145 | ASN | CB | 24.82 | 48.29 | 1.92 | 10.65 |
| 145 | ASN | CG | 25.83 | 49.40 | 2.13 | 11.01 |
| 145 | ASN | OD1 | 26.76 | 49.57 | 1.36 | 11.26 |
| 145 | ASN | ND2 | 25.64 | 50.16 | 3.20 | 11.32 |
| 145 | ASN | C | 23.51 | 46.92 | 0.33 | 10.15 |
| 145 | ASN | O | 24.06 | 45.83 | 0.14 | 10.00 |
| 146 | ILE | N | 22.19 | 47.05 | 0.45 | 9.92 |
| 146 | ILE | CA | 21.31 | 45.90 | 0.33 | 9.80 |
| 146 | ILE | CB | 19.84 | 46.27 | 0.66 | 9.83 |
| 146 | ILE | CG2 | 18.92 | 45.06 | 0.45 | 9.74 |
| 146 | ILE | CG1 | 19.74 | 46.71 | 2.13 | 10.03 |
| 146 | ILE | CD1 | 18.37 | 47.24 | 2.53 | 10.28 |
| 146 | ILE | C | 21.39 | 45.31 | −1.08 | 9.68 |
| 146 | ILE | O | 21.77 | 44.15 | −1.25 | 9.64 |
| 147 | GLY | N | 21.06 | 46.12 | −2.08 | 9.67 |
| 147 | GLY | CA | 21.12 | 45.64 | −3.45 | 9.62 |
| 147 | GLY | C | 22.52 | 45.23 | −3.87 | 9.61 |
| 147 | GLY | O | 22.71 | 44.28 | −4.63 | 9.62 |
| 148 | LYS | N | 23.52 | 45.94 | −3.36 | 9.54 |
| 148 | LYS | CA | 24.90 | 45.62 | −3.70 | 9.49 |
| 148 | LYS | CB | 25.84 | 46.62 | −3.03 | 9.74 |
| 148 | LYS | CG | 27.29 | 46.45 | −3.39 | 10.06 |
| 148 | LYS | CD | 28.11 | 47.60 | −2.82 | 10.49 |
| 148 | LYS | CE | 29.48 | 47.63 | −3.45 | 10.81 |
| 148 | LYS | NZ | 29.36 | 47.97 | −4.90 | 11.31 |
| 148 | LYS | C | 25.21 | 44.20 | −3.25 | 9.36 |
| 148 | LYS | O | 25.82 | 43.42 | −3.98 | 9.26 |
| 149 | ALA | N | 24.78 | 43.86 | −2.03 | 9.27 |
| 149 | ALA | CA | 25.02 | 42.53 | −1.48 | 9.22 |
| 149 | ALA | CB | 24.63 | 42.50 | 0.00 | 9.08 |
| 149 | ALA | C | 24.22 | 41.46 | −2.23 | 9.21 |
| 149 | ALA | O | 24.72 | 40.36 | −2.47 | 9.09 |
| 150 | VAL | N | 22.98 | 41.79 | −2.58 | 9.31 |
| 150 | VAL | CA | 22.13 | 40.86 | −3.30 | 9.42 |
| 150 | VAL | CB | 20.71 | 41.44 | −3.51 | 9.49 |
| 150 | VAL | CG1 | 19.87 | 40.49 | −4.35 | 9.56 |
| 150 | VAL | CG2 | 20.04 | 41.67 | −2.17 | 9.57 |
| 150 | VAL | C | 22.73 | 40.52 | −4.66 | 9.48 |
| 150 | VAL | O | 22.86 | 39.34 | −5.00 | 9.50 |
| 151 | HIS | N | 23.11 | 41.53 | −5.42 | 9.61 |
| 151 | HIS | CA | 23.68 | 41.30 | −6.75 | 9.73 |
| 151 | HIS | CB | 23.79 | 42.62 | −7.51 | 10.01 |
| 151 | HIS | CG | 24.18 | 42.46 | −8.95 | 10.40 |
| 151 | HIS | CD2 | 23.43 | 42.40 | −10.07 | 10.64 |
| 151 | HIS | ND1 | 25.49 | 42.34 | −9.36 | 10.64 |
| 151 | HIS | CE1 | 25.53 | 42.20 | −10.68 | 10.76 |
| 151 | HIS | NE2 | 24.29 | 42.23 | −11.13 | 10.75 |
| 151 | HIS | C | 25.04 | 40.60 | −6.70 | 9.75 |
| 151 | HIS | O | 25.34 | 39.73 | −7.53 | 9.51 |
| 152 | ASN | N | 25.86 | 40.96 | −5.71 | 9.84 |
| 152 | ASN | CA | 27.17 | 40.34 | −5.56 | 9.98 |
| 152 | ASN | CB | 27.96 | 41.04 | −4.44 | 10.11 |
| 152 | ASN | CG | 29.35 | 40.46 | −4.25 | 10.41 |
| 152 | ASN | OD1 | 29.50 | 39.26 | −4.02 | 10.73 |
| 152 | ASN | ND2 | 30.37 | 41.30 | −4.33 | 10.37 |
| 152 | ASN | C | 26.97 | 38.85 | −5.25 | 10.07 |
| 152 | ASN | O | 27.68 | 37.99 | −5.78 | 9.98 |
| 153 | THR | N | 25.99 | 38.54 | −4.40 | 10.14 |
| 153 | THR | CA | 25.71 | 37.15 | −4.05 | 10.32 |
| 153 | THR | CB | 24.60 | 37.04 | −2.98 | 10.30 |
| 153 | THR | OG1 | 24.99 | 37.75 | −1.80 | 10.34 |
| 153 | THR | CG2 | 24.34 | 35.58 | −2.63 | 10.34 |
| 153 | THR | C | 25.27 | 36.38 | −5.29 | 10.42 |
| 153 | THR | O | 25.69 | 35.24 | −5.51 | 10.42 |
| 154 | ALA | N | 24.41 | 37.00 | −6.11 | 10.56 |
| 154 | ALA | CA | 23.95 | 36.34 | −7.32 | 10.81 |
| 154 | ALA | CB | 22.94 | 37.22 | −8.05 | 10.76 |
| 154 | ALA | C | 25.15 | 36.06 | −8.22 | 10.97 |
| 154 | ALA | O | 25.31 | 34.95 | −8.74 | 11.05 |
| 155 | ARG | N | 26.00 | 37.06 | −8.40 | 11.20 |
| 155 | ARG | CA | 27.18 | 36.94 | −9.25 | 11.47 |
| 155 | ARG | CB | 27.92 | 38.28 | −9.29 | 11.94 |
| 155 | ARG | CG | 28.66 | 38.53 | −10.59 | 12.70 |
| 155 | ARG | CD | 29.76 | 37.52 | −10.80 | 13.28 |
| 155 | ARG | NE | 30.14 | 37.43 | −12.21 | 13.81 |
| 155 | ARG | CZ | 31.10 | 36.64 | −12.68 | 13.99 |
| 155 | ARG | NH1 | 31.78 | 35.85 | −11.85 | 14.19 |
| 155 | ARG | NH2 | 31.37 | 36.62 | −13.97 | 14.23 |
| 155 | ARG | C | 28.13 | 35.83 | −8.79 | 11.39 |
| 155 | ARG | O | 28.70 | 35.12 | −9.62 | 11.28 |
| 156 | GLN | N | 28.29 | 35.69 | −7.47 | 11.33 |
| 156 | GLN | CA | 29.17 | 34.66 | −6.92 | 11.30 |
| 156 | GLN | CB | 29.26 | 34.75 | −5.40 | 11.36 |
| 156 | GLN | CG | 29.81 | 36.04 | −4.85 | 11.52 |
| 156 | GLN | CD | 30.00 | 35.96 | −3.35 | 11.66 |
| 156 | GLN | OE1 | 30.04 | 36.98 | −2.65 | 11.84 |
| 156 | GLN | NE2 | 30.11 | 34.73 | −2.83 | 11.66 |
| 156 | GLN | C | 28.68 | 33.26 | −7.26 | 11.26 |
| 156 | GLN | O | 29.43 | 32.29 | −7.18 | 11.23 |
| 157 | ASN | N | 27.40 | 33.15 | −7.61 | 11.30 |
| 157 | ASN | CA | 26.83 | 31.84 | −7.94 | 11.37 |
| 157 | ASN | CB | 25.68 | 31.53 | −6.99 | 11.56 |
| 157 | ASN | CG | 26.14 | 31.44 | −5.56 | 11.71 |
| 157 | ASN | OD1 | 26.12 | 32.43 | −4.82 | 11.91 |
| 157 | ASN | ND2 | 26.57 | 30.25 | −5.16 | 11.81 |
| 157 | ASN | C | 26.37 | 31.72 | −9.38 | 11.37 |
| 157 | ASN | O | 25.50 | 30.90 | −9.69 | 11.28 |
| 158 | ASP | N | 26.94 | 32.54 | −10.24 | 11.45 |
| 158 | ASP | CA | 26.61 | 32.53 | −11.66 | 11.57 |
| 158 | ASP | CB | 27.14 | 31.24 | −12.30 | 12.05 |
| 158 | ASP | CG | 28.65 | 31.09 | −12.14 | 12.47 |
| 158 | ASP | OD1 | 29.40 | 31.93 | −12.68 | 12.83 |
| 158 | ASP | OD2 | 29.09 | 30.14 | −11.46 | 12.93 |
| 158 | ASP | C | 25.12 | 32.67 | −11.92 | 11.41 |
| 158 | ASP | O | 24.55 | 31.97 | −12.76 | 11.39 |
| 159 | LEU | N | 24.49 | 33.58 | −11.19 | 11.20 |
| 159 | LEU | CA | 23.07 | 33.83 | −11.34 | 11.00 |
| 159 | LEU | CB | 22.30 | 33.32 | −10.12 | 11.02 |
| 159 | LEU | CG | 22.30 | 31.79 | −9.97 | 11.06 |
| 159 | LEU | CD1 | 21.67 | 31.41 | −8.64 | 11.02 |
| 159 | LEU | CD2 | 21.52 | 31.17 | −11.13 | 11.05 |
| 159 | LEU | C | 22.81 | 35.32 | −11.54 | 10.86 |
| 159 | LEU | O | 23.72 | 36.14 | −11.46 | 10.85 |
| 160 | LYS | N | 21.55 | 35.65 | −11.80 | 10.79 |
| 160 | LYS | CA | 21.14 | 37.03 | −12.04 | 10.74 |
| 160 | LYS | CB | 20.56 | 37.16 | −13.45 | 10.96 |
| 160 | LYS | CG | 21.55 | 36.92 | −14.58 | 11.19 |
| 160 | LYS | CD | 22.74 | 37.86 | −14.46 | 11.58 |
| 160 | LYS | CE | 23.63 | 37.83 | −15.70 | 11.85 |
| 160 | LYS | NZ | 24.08 | 36.45 | −16.06 | 12.14 |
| 160 | LYS | C | 20.09 | 37.44 | −11.02 | 10.62 |
| 160 | LYS | O | 19.62 | 36.62 | −10.23 | 10.47 |
| 161 | VAL | N | 19.73 | 38.72 | −11.02 | 10.59 |
| 161 | VAL | CA | 18.69 | 39.20 | −10.12 | 10.59 |
| 161 | VAL | CB | 19.23 | 40.29 | −9.12 | 10.61 |
| 161 | VAL | CG1 | 20.41 | 39.74 | −8.34 | 10.70 |
| 161 | VAL | CG2 | 19.64 | 41.54 | −9.88 | 10.71 |
| 161 | VAL | C | 17.60 | 39.82 | −11.00 | 10.50 |
| 161 | VAL | O | 17.85 | 40.13 | −12.17 | 10.56 |
| 162 | ILE | N | 16.40 | 39.96 | −10.46 | 10.41 |
| 162 | ILE | CA | 15.30 | 40.59 | −11.20 | 10.41 |
| 162 | ILE | CB | 13.91 | 40.11 | −10.72 | 10.51 |
| 162 | ILE | CG2 | 12.81 | 40.89 | −11.43 | 10.39 |
| 162 | ILE | CG1 | 13.74 | 38.61 | −10.98 | 10.47 |
| 162 | ILE | CD1 | 13.82 | 38.21 | −12.44 | 10.55 |
| 162 | ILE | C | 15.48 | 42.06 | −10.86 | 10.36 |
| 162 | ILE | O | 15.32 | 42.45 | −9.71 | 10.42 |
| 163 | LYS | N | 15.82 | 42.86 | −11.86 | 10.37 |
| 163 | LYS | CA | 16.07 | 44.28 | −11.64 | 10.45 |
| 163 | LYS | CB | 16.91 | 44.84 | −12.79 | 10.71 |
| 163 | LYS | CG | 18.29 | 44.21 | −12.94 | 11.10 |
| 163 | LYS | CD | 19.00 | 44.79 | −14.15 | 11.53 |
| 163 | LYS | CE | 20.34 | 44.12 | −14.40 | 11.90 |
| 163 | LYS | NZ | 21.36 | 44.45 | −13.37 | 12.31 |
| 163 | LYS | C | 14.84 | 45.16 | −11.48 | 10.40 |
| 163 | LYS | O | 14.88 | 46.16 | −10.77 | 10.37 |
| 164 | ASN | N | 13.74 | 44.79 | −12.13 | 10.36 |
| 164 | ASN | CA | 12.54 | 45.60 | −12.04 | 10.28 |
| 164 | ASN | CB | 11.87 | 45.72 | −13.43 | 10.27 |
| 164 | ASN | CG | 11.31 | 44.40 | −13.92 | 10.25 |
| 164 | ASN | OD1 | 11.81 | 43.32 | −13.60 | 10.09 |
| 164 | ASN | ND2 | 10.26 | 44.49 | −14.74 | 10.29 |
| 164 | ASN | C | 11.54 | 45.21 | −10.97 | 10.20 |
| 164 | ASN | O | 10.36 | 45.54 | −11.05 | 10.32 |
| 165 | LEU | N | 12.01 | 44.50 | −9.95 | 10.08 |
| 165 | LEU | CA | 11.19 | 44.12 | −8.80 | 10.06 |
| 165 | LEU | CB | 10.94 | 42.61 | −8.76 | 9.99 |
| 165 | LEU | CG | 9.85 | 42.09 | −9.72 | 9.94 |
| 165 | LEU | CD1 | 9.70 | 40.59 | −9.55 | 9.76 |
| 165 | LEU | CD2 | 8.53 | 42.79 | −9.43 | 9.89 |
| 165 | LEU | C | 12.03 | 44.56 | −7.61 | 10.09 |
| 165 | LEU | O | 13.23 | 44.30 | −7.57 | 10.08 |
| 166 | THR | N | 11.41 | 45.23 | −6.64 | 10.06 |
| 166 | THR | CA | 12.16 | 45.72 | −5.48 | 10.01 |
| 166 | THR | CB | 12.50 | 47.21 | −5.65 | 9.95 |
| 166 | THR | OG1 | 11.35 | 47.99 | −5.28 | 10.06 |
| 166 | THR | CG2 | 12.83 | 47.53 | −7.10 | 9.95 |
| 166 | THR | C | 11.46 | 45.62 | −4.14 | 9.99 |
| 166 | THR | O | 10.26 | 45.34 | −4.06 | 9.93 |
| 167 | GLY | N | 12.23 | 45.88 | −3.08 | 9.94 |
| 167 | GLY | CA | 11.72 | 45.87 | −1.73 | 9.93 |
| 167 | GLY | C | 11.11 | 47.24 | −1.49 | 9.96 |
| 167 | GLY | O | 11.08 | 48.06 | −2.40 | 9.92 |
| 168 | HIS | N | 10.68 | 47.53 | −0.27 | 9.98 |
| 168 | HIS | CA | 10.03 | 48.81 | 0.00 | 9.96 |
| 168 | HIS | CB | 8.66 | 48.77 | −0.67 | 9.94 |
| 168 | HIS | CG | 7.88 | 47.54 | −0.32 | 9.79 |
| 168 | HIS | CD2 | 7.77 | 46.35 | −0.95 | 9.82 |
| 168 | HIS | ND1 | 7.19 | 47.41 | 0.87 | 9.83 |
| 168 | HIS | CE1 | 6.69 | 46.19 | 0.95 | 9.83 |
| 168 | HIS | NE2 | 7.03 | 45.53 | −0.14 | 9.82 |
| 168 | HIS | C | 9.83 | 49.02 | 1.48 | 9.97 |
| 168 | HIS | O | 9.89 | 48.08 | 2.26 | 10.06 |
| 169 | GLY | N | 9.58 | 50.27 | 1.88 | 10.07 |
| 169 | GLY | CA | 9.33 | 50.53 | 3.28 | 10.11 |
| 169 | GLY | C | 7.97 | 49.93 | 3.61 | 10.24 |
| 169 | GLY | O | 7.13 | 49.78 | 2.72 | 10.27 |
| 170 | VAL | N | 7.77 | 49.58 | 4.88 | 10.35 |
| 170 | VAL | CA | 6.50 | 49.00 | 5.32 | 10.48 |
| 170 | VAL | CB | 6.54 | 47.44 | 5.35 | 10.55 |
| 170 | VAL | CG1 | 7.60 | 46.96 | 6.30 | 10.71 |
| 170 | VAL | CG2 | 5.17 | 46.89 | 5.75 | 10.67 |
| 170 | VAL | C | 6.19 | 49.52 | 6.71 | 10.48 |
| 170 | VAL | O | 7.08 | 50.03 | 7.39 | 10.44 |
| 171 | GLY | N | 4.93 | 49.44 | 7.11 | 10.58 |
| 171 | GLY | CA | 4.55 | 49.91 | 8.44 | 10.66 |
| 171 | GLY | C | 3.08 | 50.23 | 8.51 | 10.75 |
| 171 | GLY | O | 2.29 | 49.44 | 9.04 | 10.69 |
| 172 | LEU | N | 2.70 | 51.38 | 7.98 | 10.96 |
| 172 | LEU | CA | 1.30 | 51.82 | 7.97 | 11.30 |
| 172 | LEU | CB | 1.23 | 53.31 | 8.30 | 11.37 |
| 172 | LEU | CG | 1.74 | 53.65 | 9.70 | 11.48 |
| 172 | LEU | CD1 | 1.91 | 55.16 | 9.85 | 11.51 |
| 172 | LEU | CD2 | 0.77 | 53.09 | 10.73 | 11.36 |
| 172 | LEU | C | 0.67 | 51.55 | 6.60 | 11.55 |
| 172 | LEU | O | −0.51 | 51.80 | 6.37 | 11.53 |
| 173 | SER | N | 1.50 | 51.02 | 5.71 | 11.91 |
| 173 | SER | CA | 1.11 | 50.67 | 4.35 | 12.31 |
| 173 | SER | CB | 1.08 | 51.91 | 3.45 | 12.24 |
| 173 | SER | OG | 2.40 | 52.34 | 3.16 | 12.16 |
| 173 | SER | C | 2.17 | 49.72 | 3.83 | 12.65 |
| 173 | SER | O | 3.15 | 49.42 | 4.51 | 12.60 |
| 174 | LEU | N | 1.98 | 49.27 | 2.59 | 13.10 |
| 174 | LEU | CA | 2.89 | 48.35 | 1.94 | 13.58 |
| 174 | LEU | CB | 2.09 | 47.32 | 1.14 | 13.60 |
| 174 | LEU | CG | 2.78 | 46.11 | 0.52 | 13.54 |
| 174 | LEU | CD1 | 2.52 | 44.89 | 1.41 | 13.50 |
| 174 | LEU | CD2 | 2.20 | 45.85 | −0.88 | 13.48 |
| 174 | LEU | C | 3.81 | 49.13 | 0.99 | 13.87 |
| 174 | LEU | O | 4.67 | 48.56 | 0.32 | 14.11 |
| 175 | HIS | N | 3.61 | 50.44 | 0.93 | 14.09 |
| 175 | HIS | CA | 4.44 | 51.26 | 0.07 | 14.23 |
| 175 | HIS | CB | 3.80 | 51.40 | −1.32 | 14.53 |
| 175 | HIS | CG | 4.54 | 50.65 | −2.40 | 14.68 |
| 175 | HIS | CD2 | 5.33 | 51.09 | −3.40 | 14.77 |
| 175 | HIS | ND1 | 4.55 | 49.27 | −2.48 | 14.83 |
| 175 | HIS | CE1 | 5.31 | 48.90 | −3.50 | 14.77 |
| 175 | HIS | NE2 | 5.80 | 49.98 | −4.08 | 14.82 |
| 175 | HIS | C | 4.75 | 52.64 | 0.65 | 14.13 |
| 175 | HIS | O | 4.05 | 53.62 | 0.40 | 14.31 |
| 176 | GLU | N | 5.82 | 52.69 | 1.44 | 13.85 |
| 176 | GLU | CA | 6.28 | 53.92 | 2.04 | 13.47 |
| 176 | GLU | CB | 5.79 | 54.05 | 3.48 | 13.51 |
| 176 | GLU | CG | 5.74 | 52.76 | 4.29 | 13.57 |
| 176 | GLU | CD | 4.92 | 52.91 | 5.55 | 13.66 |
| 176 | GLU | OE1 | 5.50 | 53.01 | 6.66 | 13.70 |
| 176 | GLU | OE2 | 3.67 | 52.93 | 5.44 | 13.69 |
| 176 | GLU | C | 7.79 | 54.01 | 1.97 | 13.23 |
| 176 | GLU | O | 8.45 | 53.04 | 1.59 | 13.12 |
| 177 | ALA | N | 8.35 | 55.17 | 2.30 | 13.05 |
| 177 | ALA | CA | 9.79 | 55.36 | 2.24 | 12.97 |
| 177 | ALA | CB | 10.14 | 56.76 | 2.71 | 12.80 |
| 177 | ALA | C | 10.55 | 54.33 | 3.06 | 12.90 |
| 177 | ALA | O | 10.17 | 54.02 | 4.19 | 12.93 |
| 178 | PRO | N | 11.63 | 53.75 | 2.50 | 12.91 |
| 178 | PRO | CD | 12.50 | 52.82 | 3.22 | 13.04 |
| 178 | PRO | CA | 12.15 | 54.02 | 1.16 | 12.99 |
| 178 | PRO | CB | 13.56 | 53.44 | 1.22 | 13.06 |
| 178 | PRO | CG | 13.39 | 52.27 | 2.13 | 13.08 |
| 178 | PRO | C | 11.29 | 53.36 | 0.09 | 12.94 |
| 178 | PRO | O | 10.90 | 52.20 | 0.22 | 12.99 |
| 179 | ALA | N | 10.98 | 54.11 | −0.96 | 12.77 |
| 179 | ALA | CA | 10.12 | 53.62 | −2.04 | 12.74 |
| 179 | ALA | CB | 9.97 | 54.70 | −3.11 | 12.69 |
| 179 | ALA | C | 10.60 | 52.32 | −2.69 | 12.70 |
| 179 | ALA | O | 9.80 | 51.40 | −2.89 | 12.59 |
| 180 | HIS | N | 11.88 | 52.26 | −3.01 | 12.66 |
| 180 | HIS | CA | 12.42 | 51.07 | −3.65 | 12.68 |
| 180 | HIS | CB | 12.62 | 51.33 | −5.15 | 12.86 |
| 180 | HIS | CG | 11.37 | 51.72 | −5.87 | 13.18 |
| 180 | HIS | CD2 | 10.36 | 50.96 | −6.36 | 13.25 |
| 180 | HIS | ND1 | 11.05 | 53.02 | −6.17 | 13.35 |
| 180 | HIS | CE1 | 9.90 | 53.06 | −6.81 | 13.39 |
| 180 | HIS | NE2 | 9.46 | 51.82 | −6.95 | 13.43 |
| 180 | HIS | C | 13.72 | 50.58 | −3.07 | 12.56 |
| 180 | HIS | O | 14.68 | 51.33 | −2.92 | 12.63 |
| 181 | VAL | N | 13.75 | 49.29 | −2.76 | 12.47 |
| 181 | VAL | CA | 14.94 | 48.65 | −2.23 | 12.42 |
| 181 | VAL | CB | 14.60 | 47.79 | −1.01 | 12.51 |
| 181 | VAL | CG1 | 15.86 | 47.15 | −0.45 | 12.52 |
| 181 | VAL | CG2 | 13.91 | 48.64 | 0.05 | 12.44 |
| 181 | VAL | C | 15.41 | 47.76 | −3.38 | 12.44 |
| 181 | VAL | O | 14.93 | 46.64 | −3.54 | 12.38 |
| 182 | LEU | N | 16.33 | 48.29 | −4.18 | 12.48 |
| 182 | LEU | CA | 16.83 | 47.57 | −5.35 | 12.51 |
| 182 | LEU | CB | 17.73 | 48.49 | −6.19 | 12.46 |
| 182 | LEU | CG | 17.17 | 49.84 | −6.65 | 12.52 |
| 182 | LEU | CD1 | 18.29 | 50.64 | −7.31 | 12.49 |
| 182 | LEU | CD2 | 16.01 | 49.65 | −7.62 | 12.49 |
| 182 | LEU | C | 17.61 | 46.31 | −5.02 | 12.55 |
| 182 | LEU | O | 18.12 | 46.14 | −3.92 | 12.51 |
| 183 | ASN | N | 17.67 | 45.43 | −6.01 | 12.67 |
| 183 | ASN | CA | 18.39 | 44.17 | −5.90 | 12.80 |
| 183 | ASN | CB | 17.60 | 43.05 | −6.57 | 12.86 |
| 183 | ASN | CG | 16.34 | 42.71 | −5.81 | 12.96 |
| 183 | ASN | OD1 | 16.36 | 42.57 | −4.59 | 12.95 |
| 183 | ASN | ND2 | 15.23 | 42.56 | −6.53 | 13.02 |
| 183 | ASN | C | 19.76 | 44.30 | −6.53 | 12.88 |
| 183 | ASN | O | 20.44 | 43.30 | −6.79 | 12.79 |
| 184 | TYR | N | 20.16 | 45.54 | −6.79 | 13.07 |
| 184 | TYR | CA | 21.47 | 45.80 | −7.36 | 13.25 |
| 184 | TYR | CB | 21.42 | 45.84 | −8.90 | 13.19 |
| 184 | TYR | CG | 20.50 | 46.88 | −9.46 | 13.24 |
| 184 | TYR | CD1 | 19.12 | 46.65 | −9.53 | 13.16 |
| 184 | TYR | CE1 | 18.25 | 47.62 | −10.01 | 13.27 |
| 184 | TYR | CD2 | 20.99 | 48.13 | −9.89 | 13.22 |
| 184 | TYR | CE2 | 20.12 | 49.11 | −10.37 | 13.29 |
| 184 | TYR | CZ | 18.75 | 48.85 | −10.42 | 13.29 |
| 184 | TYR | OH | 17.88 | 49.80 | −10.86 | 13.44 |
| 184 | TYR | C | 21.98 | 47.13 | −6.82 | 13.46 |
| 184 | TYR | O | 21.24 | 47.89 | −6.20 | 13.50 |
| 185 | PHE | N | 23.25 | 47.40 | −7.08 | 13.73 |
| 185 | PHE | CA | 23.91 | 48.60 | −6.59 | 14.07 |
| 185 | PHE | CB | 25.41 | 48.32 | −6.43 | 14.10 |
| 185 | PHE | CG | 26.21 | 49.51 | −5.98 | 14.16 |
| 185 | PHE | CD1 | 25.87 | 50.20 | −4.82 | 14.21 |
| 185 | PHE | CD2 | 27.32 | 49.94 | −6.71 | 14.20 |
| 185 | PHE | CE1 | 26.61 | 51.30 | −4.39 | 14.25 |
| 185 | PHE | CE2 | 28.07 | 51.04 | −6.29 | 14.32 |
| 185 | PHE | CZ | 27.71 | 51.72 | −5.13 | 14.29 |
| 185 | PHE | C | 23.71 | 49.85 | −7.45 | 14.32 |
| 185 | PHE | O | 23.91 | 49.83 | −8.66 | 14.30 |
| 186 | ASP | N | 23.30 | 50.94 | −6.79 | 14.66 |
| 186 | ASP | CA | 23.10 | 52.23 | −7.45 | 15.02 |
| 186 | ASP | CB | 21.66 | 52.73 | −7.31 | 15.11 |
| 186 | ASP | CG | 21.47 | 54.11 | −7.93 | 15.34 |
| 186 | ASP | OD1 | 22.38 | 54.57 | −8.66 | 15.50 |
| 186 | ASP | OD2 | 20.41 | 54.72 | −7.71 | 15.44 |
| 186 | ASP | C | 24.05 | 53.20 | −6.75 | 15.16 |
| 186 | ASP | O | 23.76 | 53.70 | −5.66 | 15.11 |
| 187 | PRO | N | 25.20 | 53.47 | −7.38 | 15.32 |
| 187 | PRO | CD | 25.54 | 52.99 | −8.73 | 15.36 |
| 187 | PRO | CA | 26.24 | 54.37 | −6.88 | 15.49 |
| 187 | PRO | CB | 27.32 | 54.28 | −7.95 | 15.46 |
| 187 | PRO | CG | 26.51 | 54.04 | −9.20 | 15.44 |
| 187 | PRO | C | 25.82 | 55.82 | −6.62 | 15.62 |
| 187 | PRO | O | 26.55 | 56.57 | −5.97 | 15.69 |
| 188 | LYS | N | 24.64 | 56.22 | −7.10 | 15.72 |
| 188 | LYS | CA | 24.18 | 57.59 | −6.90 | 15.81 |
| 188 | LYS | CB | 23.41 | 58.09 | −8.12 | 16.04 |
| 188 | LYS | CG | 22.03 | 57.48 | −8.22 | 16.29 |
| 188 | LYS | CD | 21.13 | 58.20 | −9.21 | 16.52 |
| 188 | LYS | CE | 19.68 | 57.78 | −8.96 | 16.61 |
| 188 | LYS | NZ | 19.32 | 58.05 | −7.54 | 16.74 |
| 188 | LYS | C | 23.29 | 57.77 | −5.67 | 15.77 |
| 188 | LYS | O | 23.10 | 58.89 | −5.19 | 15.78 |
| 189 | ASP | N | 22.72 | 56.67 | −5.18 | 15.64 |
| 189 | ASP | CA | 21.82 | 56.75 | −4.03 | 15.56 |
| 189 | ASP | CB | 21.01 | 55.45 | −3.90 | 15.65 |
| 189 | ASP | CG | 19.95 | 55.54 | −2.83 | 15.79 |
| 189 | ASP | OD1 | 19.62 | 56.67 | −2.40 | 15.80 |
| 189 | ASP | OD2 | 19.42 | 54.48 | −2.44 | 15.85 |
| 189 | ASP | C | 22.55 | 57.02 | −2.72 | 15.40 |
| 189 | ASP | O | 23.46 | 56.29 | −2.34 | 15.37 |
| 190 | LYS | N | 22.14 | 58.09 | −2.04 | 15.25 |
| 190 | LYS | CA | 22.77 | 58.45 | −0.77 | 15.04 |
| 190 | LYS | CB | 23.17 | 59.93 | −0.80 | 15.21 |
| 190 | LYS | CG | 24.21 | 60.27 | −1.87 | 15.49 |
| 190 | LYS | CD | 24.56 | 61.75 | −1.85 | 15.72 |
| 190 | LYS | CE | 25.54 | 62.12 | −2.95 | 15.89 |
| 190 | LYS | NZ | 25.76 | 63.59 | −3.05 | 16.07 |
| 190 | LYS | C | 21.82 | 58.19 | 0.40 | 14.76 |
| 190 | LYS | O | 22.11 | 58.55 | 1.54 | 14.81 |
| 191 | THR | N | 20.68 | 57.57 | 0.11 | 14.44 |
| 191 | THR | CA | 19.70 | 57.27 | 1.15 | 14.10 |
| 191 | THR | CB | 18.46 | 56.56 | 0.57 | 14.26 |
| 191 | THR | OG1 | 17.91 | 57.36 | −0.48 | 14.42 |
| 191 | THR | CG2 | 17.41 | 56.38 | 1.66 | 14.37 |
| 191 | THR | C | 20.30 | 56.37 | 2.21 | 13.73 |
| 191 | THR | O | 20.98 | 55.39 | 1.89 | 13.66 |
| 192 | LEU | N | 20.06 | 56.71 | 3.46 | 13.41 |
| 192 | LEU | CA | 20.58 | 55.93 | 4.57 | 13.11 |
| 192 | LEU | CB | 21.39 | 56.82 | 5.51 | 13.24 |
| 192 | LEU | CG | 22.58 | 57.58 | 4.93 | 13.32 |
| 192 | LEU | CD1 | 23.24 | 58.38 | 6.05 | 13.46 |
| 192 | LEU | CD2 | 23.57 | 56.60 | 4.32 | 13.42 |
| 192 | LEU | C | 19.48 | 55.28 | 5.38 | 12.86 |
| 192 | LEU | O | 18.34 | 55.75 | 5.39 | 12.71 |
| 193 | LEU | N | 19.82 | 54.19 | 6.06 | 12.74 |
| 193 | LEU | CA | 18.87 | 53.49 | 6.91 | 12.69 |
| 193 | LEU | CB | 19.25 | 52.02 | 7.06 | 12.81 |
| 193 | LEU | CG | 19.42 | 51.23 | 5.76 | 12.91 |
| 193 | LEU | CD1 | 19.69 | 49.77 | 6.08 | 13.00 |
| 193 | LEU | CD2 | 18.16 | 51.36 | 4.92 | 13.04 |
| 193 | LEU | C | 18.99 | 54.20 | 8.25 | 12.63 |
| 193 | LEU | O | 20.07 | 54.66 | 8.62 | 12.63 |
| 194 | THR | N | 17.88 | 54.30 | 8.97 | 12.54 |
| 194 | THR | CA | 17.88 | 54.97 | 10.27 | 12.52 |
| 194 | THR | CB | 17.06 | 56.28 | 10.20 | 12.60 |
| 194 | THR | OG1 | 15.71 | 55.98 | 9.85 | 12.63 |
| 194 | THR | CG2 | 17.65 | 57.23 | 9.16 | 12.67 |
| 194 | THR | C | 17.30 | 54.09 | 11.37 | 12.44 |
| 194 | THR | O | 16.62 | 53.10 | 11.10 | 12.36 |
| 195 | GLU | N | 17.59 | 54.46 | 12.61 | 12.40 |
| 195 | GLU | CA | 17.10 | 53.73 | 13.77 | 12.33 |
| 195 | GLU | CB | 17.60 | 54.43 | 15.04 | 12.55 |
| 195 | GLU | CG | 17.09 | 53.86 | 16.36 | 12.99 |
| 195 | GLU | CD | 17.58 | 52.46 | 16.64 | 13.22 |
| 195 | GLU | OE1 | 18.76 | 52.16 | 16.33 | 13.44 |
| 195 | GLU | OE2 | 16.80 | 51.65 | 17.19 | 13.41 |
| 195 | GLU | C | 15.57 | 53.70 | 13.74 | 12.13 |
| 195 | GLU | O | 14.92 | 54.74 | 13.64 | 12.09 |
| 196 | GLY | N | 15.00 | 52.50 | 13.80 | 11.89 |
| 196 | GLY | CA | 13.55 | 52.36 | 13.78 | 11.57 |
| 196 | GLY | C | 12.92 | 52.21 | 12.40 | 11.42 |
| 196 | GLY | O | 11.72 | 51.97 | 12.29 | 11.33 |
| 197 | MET | N | 13.72 | 52.34 | 11.35 | 11.29 |
| 197 | MET | CA | 13.20 | 52.22 | 9.99 | 11.09 |
| 197 | MET | CB | 14.27 | 52.62 | 8.97 | 11.33 |
| 197 | MET | CG | 13.75 | 52.72 | 7.55 | 11.61 |
| 197 | MET | SD | 15.05 | 53.09 | 6.35 | 11.77 |
| 197 | MET | CE | 14.94 | 54.87 | 6.31 | 11.87 |
| 197 | MET | C | 12.73 | 50.80 | 9.71 | 10.84 |
| 197 | MET | O | 13.46 | 49.84 | 9.99 | 10.76 |
| 198 | VAL | N | 11.53 | 50.66 | 9.18 | 10.65 |
| 198 | VAL | CA | 10.98 | 49.34 | 8.87 | 10.51 |
| 198 | VAL | CB | 9.55 | 49.16 | 9.44 | 10.52 |
| 198 | VAL | CG1 | 9.08 | 47.73 | 9.25 | 10.52 |
| 198 | VAL | CG2 | 9.55 | 49.52 | 10.91 | 10.49 |
| 198 | VAL | C | 10.96 | 49.16 | 7.35 | 10.47 |
| 198 | VAL | O | 10.38 | 49.98 | 6.62 | 10.40 |
| 199 | LEU | N | 11.59 | 48.08 | 6.89 | 10.50 |
| 199 | LEU | CA | 11.71 | 47.80 | 5.47 | 10.40 |
| 199 | LEU | CB | 13.14 | 48.04 | 5.00 | 10.56 |
| 199 | LEU | CG | 13.95 | 49.14 | 5.66 | 10.91 |
| 199 | LEU | CD1 | 15.36 | 49.16 | 5.08 | 11.00 |
| 199 | LEU | CD2 | 13.25 | 50.47 | 5.38 | 11.08 |
| 199 | LEU | C | 11.37 | 46.36 | 5.13 | 10.16 |
| 199 | LEU | O | 11.48 | 45.47 | 5.97 | 10.18 |
| 200 | ALA | N | 10.96 | 46.15 | 3.89 | 9.98 |
| 200 | ALA | CA | 10.68 | 44.81 | 3.41 | 9.74 |
| 200 | ALA | CB | 9.36 | 44.77 | 2.66 | 9.83 |
| 200 | ALA | C | 11.84 | 44.55 | 2.46 | 9.50 |
| 200 | ALA | O | 12.05 | 45.30 | 1.50 | 9.58 |
| 201 | ILE | N | 12.62 | 43.52 | 2.74 | 9.35 |
| 201 | ILE | CA | 13.75 | 43.18 | 1.89 | 9.18 |
| 201 | ILE | CB | 15.02 | 42.96 | 2.74 | 9.27 |
| 201 | ILE | CG2 | 16.17 | 42.47 | 1.86 | 9.08 |
| 201 | ILE | CG1 | 15.38 | 44.28 | 3.44 | 9.24 |
| 201 | ILE | CD1 | 16.71 | 44.27 | 4.19 | 9.46 |
| 201 | ILE | C | 13.35 | 41.90 | 1.16 | 9.04 |
| 201 | ILE | O | 13.04 | 40.89 | 1.79 | 8.99 |
| 202 | GLU | N | 13.33 | 41.94 | −0.17 | 9.05 |
| 202 | GLU | CA | 12.92 | 40.77 | −0.94 | 9.03 |
| 202 | GLU | CB | 11.43 | 40.88 | −1.33 | 9.20 |
| 202 | GLU | CG | 10.97 | 42.26 | −1.78 | 9.45 |
| 202 | GLU | CD | 9.45 | 42.35 | −1.99 | 9.60 |
| 202 | GLU | OE1 | 8.88 | 43.41 | −1.67 | 9.74 |
| 202 | GLU | OE2 | 8.82 | 41.40 | −2.48 | 9.74 |
| 202 | GLU | C | 13.76 | 40.48 | −2.17 | 8.86 |
| 202 | GLU | O | 13.36 | 40.78 | −3.30 | 8.91 |
| 203 | PRO | N | 14.94 | 39.89 | −1.98 | 8.68 |
| 203 | PRO | CD | 15.60 | 39.46 | −0.74 | 8.71 |
| 203 | PRO | CA | 15.77 | 39.59 | −3.15 | 8.67 |
| 203 | PRO | CB | 17.07 | 39.06 | −2.54 | 8.70 |
| 203 | PRO | CG | 16.62 | 38.45 | −1.24 | 8.72 |
| 203 | PRO | C | 15.10 | 38.57 | −4.07 | 8.49 |
| 203 | PRO | O | 14.44 | 37.64 | −3.60 | 8.45 |
| 204 | PHE | N | 15.24 | 38.79 | −5.38 | 8.36 |
| 204 | PHE | CA | 14.71 | 37.89 | −6.40 | 8.35 |
| 204 | PHE | CB | 13.79 | 38.61 | −7.39 | 8.34 |
| 204 | PHE | CG | 12.43 | 38.91 | −6.84 | 8.45 |
| 204 | PHE | CD1 | 12.22 | 40.00 | −6.01 | 8.45 |
| 204 | PHE | CD2 | 11.34 | 38.09 | −7.16 | 8.47 |
| 204 | PHE | CE1 | 10.96 | 40.28 | −5.49 | 8.57 |
| 204 | PHE | CE2 | 10.08 | 38.36 | −6.64 | 8.54 |
| 204 | PHE | CZ | 9.89 | 39.45 | −5.81 | 8.52 |
| 204 | PHE | C | 15.94 | 37.41 | −7.17 | 8.28 |
| 204 | PHE | O | 16.62 | 38.22 | −7.78 | 8.21 |
| 205 | ILE | N | 16.21 | 36.11 | −7.12 | 8.26 |
| 205 | ILE | CA | 17.35 | 35.52 | −7.82 | 8.22 |
| 205 | ILE | CB | 18.14 | 34.59 | −6.87 | 8.27 |
| 205 | ILE | CG2 | 19.27 | 33.90 | −7.63 | 8.20 |
| 205 | ILE | CG1 | 18.69 | 35.39 | −5.69 | 8.24 |
| 205 | ILE | CD1 | 19.61 | 36.53 | −6.09 | 8.30 |
| 205 | ILE | C | 16.83 | 34.74 | −9.02 | 8.16 |
| 205 | ILE | O | 15.92 | 33.92 | −8.88 | 8.29 |
| 206 | SER | N | 17.42 | 34.98 | −10.20 | 8.11 |
| 206 | SER | CA | 16.98 | 34.35 | −11.45 | 8.25 |
| 206 | SER | CB | 16.53 | 35.44 | −12.41 | 8.34 |
| 206 | SER | OG | 16.29 | 34.92 | −13.71 | 8.32 |
| 206 | SER | C | 18.02 | 33.46 | −12.15 | 8.34 |
| 206 | SER | O | 19.21 | 33.75 | −12.10 | 8.18 |
| 207 | SER | N | 17.57 | 32.40 | −12.80 | 8.58 |
| 207 | SER | CA | 18.48 | 31.51 | −13.52 | 8.76 |
| 207 | SER | CB | 17.82 | 30.16 | −13.80 | 8.62 |
| 207 | SER | OG | 16.63 | 30.31 | −14.57 | 8.68 |
| 207 | SER | C | 18.97 | 32.11 | −14.84 | 8.92 |
| 207 | SER | O | 19.94 | 31.63 | −15.42 | 8.87 |
| 208 | ASN | N | 18.29 | 33.15 | −15.32 | 9.17 |
| 208 | ASN | CA | 18.70 | 33.79 | −16.57 | 9.46 |
| 208 | ASN | CB | 18.30 | 32.93 | −17.77 | 9.81 |
| 208 | ASN | CG | 18.84 | 33.47 | −19.08 | 9.97 |
| 208 | ASN | OD1 | 18.08 | 33.88 | −19.96 | 10.29 |
| 208 | ASN | ND2 | 20.15 | 33.47 | −19.22 | 10.13 |
| 208 | ASN | C | 18.19 | 35.22 | −16.79 | 9.54 |
| 208 | ASN | O | 18.98 | 36.15 | −16.97 | 9.60 |
| 209 | ALA | N | 16.87 | 35.39 | −16.79 | 9.59 |
| 209 | ALA | CA | 16.26 | 36.70 | −17.00 | 9.69 |
| 209 | ALA | CB | 14.75 | 36.56 | −17.09 | 9.68 |
| 209 | ALA | C | 16.62 | 37.72 | −15.93 | 9.77 |
| 209 | ALA | O | 16.70 | 37.39 | −14.75 | 9.75 |
| 210 | SER | N | 16.85 | 38.97 | −16.34 | 9.91 |
| 210 | SER | CA | 17.20 | 40.03 | −15.40 | 9.94 |
| 210 | SER | CB | 18.40 | 40.81 | −15.91 | 10.24 |
| 210 | SER | OG | 18.09 | 41.53 | −17.09 | 10.82 |
| 210 | SER | C | 16.03 | 40.98 | −15.16 | 9.77 |
| 210 | SER | O | 16.20 | 42.05 | −14.58 | 9.75 |
| 211 | PHE | N | 14.84 | 40.59 | −15.61 | 9.72 |
| 211 | PHE | CA | 13.64 | 41.40 | −15.44 | 9.80 |
| 211 | PHE | CB | 13.63 | 42.57 | −16.42 | 9.74 |
| 211 | PHE | CG | 13.56 | 42.15 | −17.87 | 9.86 |
| 211 | PHE | CD1 | 14.70 | 41.73 | −18.54 | 9.91 |
| 211 | PHE | CD2 | 12.35 | 42.15 | −18.55 | 9.95 |
| 211 | PHE | CE1 | 14.63 | 41.31 | −19.87 | 10.07 |
| 211 | PHE | CE2 | 12.27 | 41.74 | −19.88 | 10.05 |
| 211 | PHE | CZ | 13.42 | 41.33 | −20.54 | 10.07 |
| 211 | PHE | C | 12.40 | 40.55 | −15.69 | 9.95 |
| 211 | PHE | O | 12.49 | 39.45 | −16.23 | 9.81 |
| 212 | VAL | N | 11.24 | 41.05 | −15.27 | 10.40 |
| 212 | VAL | CA | 9.97 | 40.35 | −15.48 | 10.92 |
| 212 | VAL | CB | 9.23 | 40.03 | −14.15 | 10.99 |
| 212 | VAL | CG1 | 10.06 | 39.07 | −13.30 | 11.17 |
| 212 | VAL | CG2 | 8.94 | 41.31 | −13.39 | 11.16 |
| 212 | VAL | C | 9.05 | 41.21 | −16.34 | 11.16 |
| 212 | VAL | O | 9.24 | 42.42 | −16.46 | 11.11 |
| 213 | THR | N | 8.05 | 40.57 | −16.93 | 11.54 |
| 213 | THR | CA | 7.08 | 41.26 | −17.77 | 12.05 |
| 213 | THR | CB | 7.33 | 40.99 | −19.27 | 12.08 |
| 213 | THR | OG1 | 7.41 | 39.57 | −19.49 | 11.95 |
| 213 | THR | CG2 | 8.62 | 41.65 | −19.72 | 12.14 |
| 213 | THR | C | 5.65 | 40.84 | −17.43 | 12.30 |
| 213 | THR | O | 5.44 | 39.86 | −16.71 | 12.36 |
| 214 | GLU | N | 4.68 | 41.58 | −17.94 | 12.79 |
| 214 | GLU | CA | 3.26 | 41.29 | −17.70 | 13.29 |
| 214 | GLU | CB | 2.39 | 42.26 | −18.50 | 13.67 |
| 214 | GLU | CG | 0.91 | 42.16 | −18.22 | 14.26 |
| 214 | GLU | CD | 0.12 | 43.24 | −18.95 | 14.60 |
| 214 | GLU | OE1 | 0.50 | 44.42 | −18.82 | 14.94 |
| 214 | GLU | OE2 | −0.86 | 42.90 | −19.64 | 14.92 |
| 214 | GLU | C | 2.98 | 39.85 | −18.14 | 13.42 |
| 214 | GLU | O | 3.42 | 39.43 | −19.21 | 13.54 |
| 215 | GLY | N | 2.25 | 39.11 | −17.32 | 13.58 |
| 215 | GLY | CA | 1.96 | 37.73 | −17.63 | 13.89 |
| 215 | GLY | C | 0.61 | 37.41 | −18.24 | 14.12 |
| 215 | GLY | O | 0.03 | 38.23 | −18.96 | 14.08 |
| 216 | LYS | N | 0.11 | 36.22 | −17.93 | 14.31 |
| 216 | LYS | CA | −1.17 | 35.72 | −18.45 | 14.45 |
| 216 | LYS | CB | −1.38 | 34.27 | −18.01 | 14.41 |
| 216 | LYS | CG | −1.55 | 34.10 | −16.50 | 14.33 |
| 216 | LYS | CD | −1.74 | 32.64 | −16.10 | 14.31 |
| 216 | LYS | CE | −0.52 | 31.78 | −16.42 | 14.35 |
| 216 | LYS | NZ | 0.74 | 32.29 | −15.79 | 14.28 |
| 216 | LYS | C | −2.40 | 36.54 | −18.07 | 14.58 |
| 216 | LYS | O | −3.48 | 36.33 | −18.62 | 14.64 |
| 217 | ASN | N | −2.24 | 37.47 | −17.13 | 14.65 |
| 217 | ASN | CA | −3.35 | 38.32 | −16.72 | 14.74 |
| 217 | ASN | CB | −4.14 | 37.69 | −15.57 | 14.74 |
| 217 | ASN | CG | −3.26 | 37.34 | −14.39 | 14.62 |
| 217 | ASN | OD1 | −2.54 | 38.19 | −13.85 | 14.62 |
| 217 | ASN | ND2 | −3.31 | 36.07 | −13.97 | 14.55 |
| 217 | ASN | C | −2.83 | 39.69 | −16.31 | 14.77 |
| 217 | ASN | O | −1.64 | 39.95 | −16.40 | 14.86 |
| 218 | GLU | N | −3.72 | 40.57 | −15.86 | 14.85 |
| 218 | GLU | CA | −3.33 | 41.92 | −15.48 | 14.88 |
| 218 | GLU | CB | −4.55 | 42.84 | −15.48 | 15.23 |
| 218 | GLU | CG | −5.68 | 42.36 | −14.58 | 15.82 |
| 218 | GLU | CD | −6.83 | 43.36 | −14.50 | 16.20 |
| 218 | GLU | OE1 | −7.30 | 43.83 | −15.56 | 16.52 |
| 218 | GLU | OE2 | −7.26 | 43.66 | −13.36 | 16.42 |
| 218 | GLU | C | −2.63 | 42.01 | −14.13 | 14.67 |
| 218 | GLU | O | −2.32 | 43.10 | −13.67 | 14.70 |
| 219 | TRP | N | −2.37 | 40.87 | −13.50 | 14.43 |
| 219 | TRP | CA | −1.71 | 40.88 | −12.20 | 14.16 |
| 219 | TRP | CB | −2.59 | 40.22 | −11.15 | 14.13 |
| 219 | TRP | CG | −3.76 | 41.05 | −10.73 | 14.22 |
| 219 | TRP | CD2 | −3.74 | 42.15 | −9.82 | 14.15 |
| 219 | TRP | CE2 | −5.07 | 42.60 | −9.67 | 14.32 |
| 219 | TRP | CE3 | −2.72 | 42.80 | −9.10 | 14.31 |
| 219 | TRP | CD1 | −5.07 | 40.88 | −11.10 | 14.15 |
| 219 | TRP | NE1 | −5.86 | 41.81 | −10.47 | 14.24 |
| 219 | TRP | CZ2 | −5.42 | 43.67 | −8.85 | 14.38 |
| 219 | TRP | CZ3 | −3.06 | 43.87 | −8.28 | 14.41 |
| 219 | TRP | CH2 | −4.40 | 44.29 | −8.16 | 14.42 |
| 219 | TRP | C | −0.34 | 40.21 | −12.18 | 13.99 |
| 219 | TRP | O | 0.66 | 40.84 | −11.83 | 13.90 |
| 220 | ALA | N | −0.29 | 38.94 | −12.54 | 13.87 |
| 220 | ALA | CA | 0.96 | 38.18 | −12.51 | 13.64 |
| 220 | ALA | CB | 0.68 | 36.72 | −12.86 | 13.82 |
| 220 | ALA | C | 2.09 | 38.71 | −13.39 | 13.49 |
| 220 | ALA | O | 1.86 | 39.23 | −14.48 | 13.51 |
| 221 | PHE | N | 3.30 | 38.59 | −12.86 | 13.28 |
| 221 | PHE | CA | 4.52 | 38.97 | −13.57 | 13.05 |
| 221 | PHE | CB | 5.42 | 39.86 | −12.71 | 13.33 |
| 221 | PHE | CG | 4.82 | 41.19 | −12.39 | 13.52 |
| 221 | PHE | CD1 | 4.21 | 41.95 | −13.38 | 13.64 |
| 221 | PHE | CD2 | 4.87 | 41.69 | −11.09 | 13.60 |
| 221 | PHE | CE1 | 3.64 | 43.19 | −13.09 | 13.72 |
| 221 | PHE | CE2 | 4.31 | 42.93 | −10.79 | 13.67 |
| 221 | PHE | CZ | 3.70 | 43.68 | −11.79 | 13.73 |
| 221 | PHE | C | 5.23 | 37.66 | −13.87 | 12.74 |
| 221 | PHE | O | 5.28 | 36.77 | −13.02 | 12.83 |
| 222 | GLU | N | 5.77 | 37.55 | −15.07 | 12.46 |
| 222 | GLU | CA | 6.45 | 36.34 | −15.52 | 12.17 |
| 222 | GLU | CB | 5.50 | 35.56 | −16.44 | 12.26 |
| 222 | GLU | CG | 4.18 | 35.10 | −15.79 | 12.32 |
| 222 | GLU | CD | 3.18 | 34.52 | −16.78 | 12.49 |
| 222 | GLU | OE1 | 3.62 | 33.97 | −17.82 | 12.51 |
| 222 | GLU | OE2 | 1.95 | 34.59 | −16.52 | 12.55 |
| 222 | GLU | C | 7.73 | 36.66 | −16.29 | 11.98 |
| 222 | GLU | O | 8.16 | 37.81 | −16.36 | 11.86 |
| 223 | THR | N | 8.36 | 35.62 | −16.84 | 11.84 |
| 223 | THR | CA | 9.54 | 35.78 | −17.68 | 11.67 |
| 223 | THR | CB | 10.85 | 35.20 | −17.06 | 11.61 |
| 223 | THR | OG1 | 10.72 | 33.79 | −16.86 | 11.66 |
| 223 | THR | CG2 | 11.19 | 35.89 | −15.75 | 11.56 |
| 223 | THR | C | 9.21 | 34.99 | −18.93 | 11.60 |
| 223 | THR | O | 8.74 | 33.85 | −18.84 | 11.61 |
| 224 | SER | N | 9.43 | 35.59 | −20.10 | 11.51 |
| 224 | SER | CA | 9.14 | 34.91 | −21.35 | 11.46 |
| 224 | SER | CB | 9.32 | 35.87 | −22.53 | 11.65 |
| 224 | SER | OG | 8.30 | 36.85 | −22.53 | 11.85 |
| 224 | SER | C | 10.05 | 33.69 | −21.54 | 11.33 |
| 224 | SER | O | 9.62 | 32.70 | −22.12 | 11.25 |
| 225 | ASP | N | 11.28 | 33.76 | −21.04 | 11.20 |
| 225 | ASP | CA | 12.18 | 32.63 | −21.19 | 11.05 |
| 225 | ASP | CB | 13.66 | 33.07 | −21.21 | 11.01 |
| 225 | ASP | CG | 14.11 | 33.72 | −19.92 | 11.17 |
| 225 | ASP | OD1 | 13.38 | 33.67 | −18.91 | 11.02 |
| 225 | ASP | OD2 | 15.23 | 34.28 | −19.91 | 11.24 |
| 225 | ASP | C | 11.95 | 31.58 | −20.11 | 10.92 |
| 225 | ASP | O | 12.68 | 30.59 | −20.02 | 10.88 |
| 226 | LYS | N | 10.92 | 31.79 | −19.30 | 10.88 |
| 226 | LYS | CA | 10.56 | 30.86 | −18.24 | 10.78 |
| 226 | LYS | CB | 9.94 | 29.60 | −18.85 | 11.09 |
| 226 | LYS | CG | 8.62 | 29.89 | −19.56 | 11.51 |
| 226 | LYS | CD | 8.09 | 28.68 | −20.31 | 12.10 |
| 226 | LYS | CE | 6.72 | 28.98 | −20.89 | 12.39 |
| 226 | LYS | NZ | 6.71 | 30.30 | −21.59 | 12.93 |
| 226 | LYS | C | 11.73 | 30.48 | −17.33 | 10.60 |
| 226 | LYS | O | 11.92 | 29.31 | −17.00 | 10.56 |
| 227 | SER | N | 12.49 | 31.49 | −16.93 | 10.51 |
| 227 | SER | CA | 13.63 | 31.27 | −16.04 | 10.35 |
| 227 | SER | CB | 14.41 | 32.57 | −15.82 | 10.72 |
| 227 | SER | OG | 15.10 | 32.98 | −17.00 | 11.09 |
| 227 | SER | C | 13.07 | 30.81 | −14.70 | 10.05 |
| 227 | SER | O | 11.88 | 30.98 | −14.43 | 10.07 |
| 228 | PHE | N | 13.93 | 30.23 | −13.87 | 9.79 |
| 228 | PHE | CA | 13.52 | 29.82 | −12.54 | 9.55 |
| 228 | PHE | CB | 14.26 | 28.54 | −12.12 | 9.79 |
| 228 | PHE | CG | 14.03 | 27.39 | −13.06 | 9.98 |
| 228 | PHE | CD1 | 12.77 | 27.15 | −13.58 | 10.13 |
| 228 | PHE | CD2 | 15.07 | 26.55 | −13.42 | 10.18 |
| 228 | PHE | CE1 | 12.54 | 26.09 | −14.46 | 10.28 |
| 228 | PHE | CE2 | 14.86 | 25.48 | −14.30 | 10.25 |
| 228 | PHE | CZ | 13.59 | 25.26 | −14.82 | 10.31 |
| 228 | PHE | C | 13.93 | 30.96 | −11.63 | 9.31 |
| 228 | PHE | O | 15.10 | 31.32 | −11.56 | 9.16 |
| 229 | VAL | N | 12.96 | 31.55 | −10.95 | 9.25 |
| 229 | VAL | CA | 13.23 | 32.68 | −10.08 | 9.07 |
| 229 | VAL | CB | 12.51 | 33.95 | −10.59 | 9.03 |
| 229 | VAL | CG1 | 12.83 | 35.13 | −9.68 | 9.02 |
| 229 | VAL | CG2 | 12.95 | 34.24 | −12.02 | 9.11 |
| 229 | VAL | C | 12.76 | 32.39 | −8.66 | 8.93 |
| 229 | VAL | O | 11.67 | 31.86 | −8.46 | 9.04 |
| 230 | ALA | N | 13.59 | 32.73 | −7.68 | 8.78 |
| 230 | ALA | CA | 13.24 | 32.51 | −6.29 | 8.62 |
| 230 | ALA | CB | 14.18 | 31.47 | −5.65 | 8.70 |
| 230 | ALA | C | 13.30 | 33.81 | −5.50 | 8.54 |
| 230 | ALA | O | 14.19 | 34.64 | −5.72 | 8.52 |
| 231 | GLN | N | 12.35 | 33.98 | −4.59 | 8.47 |
| 231 | GLN | CA | 12.27 | 35.15 | −3.72 | 8.45 |
| 231 | GLN | CB | 10.95 | 35.90 | −3.98 | 8.54 |
| 231 | GLN | CG | 10.79 | 37.23 | −3.22 | 8.84 |
| 231 | GLN | CD | 9.45 | 37.36 | −2.47 | 9.00 |
| 231 | GLN | OE1 | 8.90 | 38.46 | −2.34 | 9.22 |
| 231 | GLN | NE2 | 8.94 | 36.24 | −1.97 | 8.95 |
| 231 | GLN | C | 12.28 | 34.71 | −2.26 | 8.39 |
| 231 | GLN | O | 11.85 | 33.60 | −1.93 | 8.20 |
| 232 | ILE | N | 12.80 | 35.58 | −1.40 | 8.41 |
| 232 | ILE | CA | 12.82 | 35.38 | 0.05 | 8.44 |
| 232 | ILE | CB | 14.21 | 34.94 | 0.61 | 8.55 |
| 232 | ILE | CG2 | 14.21 | 35.04 | 2.14 | 8.79 |
| 232 | ILE | CG1 | 14.53 | 33.50 | 0.20 | 8.88 |
| 232 | ILE | CD1 | 13.52 | 32.47 | 0.70 | 9.18 |
| 232 | ILE | C | 12.54 | 36.80 | 0.55 | 8.25 |
| 232 | ILE | O | 13.28 | 37.72 | 0.23 | 8.25 |
| 233 | GLU | N | 11.46 | 36.98 | 1.30 | 8.17 |
| 233 | GLU | CA | 11.14 | 38.30 | 1.80 | 8.10 |
| 233 | GLU | CB | 9.95 | 38.88 | 1.03 | 8.32 |
| 233 | GLU | CG | 9.37 | 40.17 | 1.61 | 8.52 |
| 233 | GLU | CD | 8.23 | 40.73 | 0.77 | 8.67 |
| 233 | GLU | OE1 | 7.42 | 39.92 | 0.27 | 8.77 |
| 233 | GLU | OE2 | 8.14 | 41.97 | 0.61 | 8.88 |
| 233 | GLU | C | 10.82 | 38.29 | 3.29 | 8.00 |
| 233 | GLU | O | 10.18 | 37.37 | 3.81 | 7.98 |
| 234 | HIS | N | 11.30 | 39.33 | 3.98 | 8.06 |
| 234 | HIS | CA | 11.06 | 39.48 | 5.40 | 8.07 |
| 234 | HIS | CB | 12.26 | 38.97 | 6.21 | 8.29 |
| 234 | HIS | CG | 12.23 | 37.49 | 6.47 | 8.34 |
| 234 | HIS | CD2 | 13.01 | 36.48 | 6.00 | 8.48 |
| 234 | HIS | ND1 | 11.31 | 36.90 | 7.31 | 8.45 |
| 234 | HIS | CE1 | 11.52 | 35.60 | 7.35 | 8.52 |
| 234 | HIS | NE2 | 12.55 | 35.32 | 6.56 | 8.49 |
| 234 | HIS | C | 10.87 | 40.96 | 5.68 | 8.05 |
| 234 | HIS | O | 11.41 | 41.82 | 4.98 | 7.87 |
| 235 | THR | N | 10.07 | 41.24 | 6.70 | 8.21 |
| 235 | THR | CA | 9.85 | 42.62 | 7.13 | 8.40 |
| 235 | THR | CB | 8.43 | 42.84 | 7.64 | 8.25 |
| 235 | THR | OG1 | 7.51 | 42.74 | 6.54 | 8.13 |
| 235 | THR | CG2 | 8.29 | 44.23 | 8.27 | 8.27 |
| 235 | THR | C | 10.89 | 42.79 | 8.23 | 8.49 |
| 235 | THR | O | 10.95 | 42.00 | 9.17 | 8.42 |
| 236 | VAL | N | 11.71 | 43.83 | 8.07 | 8.84 |
| 236 | VAL | CA | 12.81 | 44.11 | 8.98 | 9.36 |
| 236 | VAL | CB | 14.14 | 44.00 | 8.21 | 9.30 |
| 236 | VAL | CG1 | 15.31 | 44.22 | 9.15 | 9.41 |
| 236 | VAL | CG2 | 14.23 | 42.64 | 7.53 | 9.40 |
| 236 | VAL | C | 12.76 | 45.48 | 9.63 | 9.38 |
| 236 | VAL | O | 12.31 | 46.44 | 9.01 | 9.51 |
| 237 | ILE | N | 13.20 | 45.56 | 10.88 | 9.82 |
| 237 | ILE | CA | 13.26 | 46.83 | 11.58 | 10.45 |
| 237 | ILE | CB | 12.45 | 46.80 | 12.89 | 10.41 |
| 237 | ILE | CG2 | 12.44 | 48.20 | 13.51 | 10.29 |
| 237 | ILE | CG1 | 11.01 | 46.36 | 12.60 | 10.51 |
| 237 | ILE | CD1 | 10.11 | 46.32 | 13.83 | 10.86 |
| 237 | ILE | C | 14.73 | 47.06 | 11.89 | 10.59 |
| 237 | ILE | O | 15.41 | 46.18 | 12.42 | 10.62 |
| 238 | VAL | N | 15.23 | 48.24 | 11.52 | 11.06 |
| 238 | VAL | CA | 16.62 | 48.62 | 11.75 | 11.85 |
| 238 | VAL | CB | 17.07 | 49.73 | 10.77 | 11.74 |
| 238 | VAL | CG1 | 18.55 | 50.04 | 10.97 | 11.75 |
| 238 | VAL | CG2 | 16.80 | 49.32 | 9.34 | 11.79 |
| 238 | VAL | C | 16.78 | 49.13 | 13.18 | 12.12 |
| 238 | VAL | O | 16.14 | 50.11 | 13.56 | 12.18 |
| 239 | THR | N | 17.62 | 48.47 | 13.96 | 12.65 |
| 239 | THR | CA | 17.87 | 48.89 | 15.34 | 13.26 |
| 239 | THR | CB | 17.19 | 47.95 | 16.37 | 13.14 |
| 239 | THR | OG1 | 18.07 | 46.87 | 16.69 | 13.43 |
| 239 | THR | CG2 | 15.89 | 47.40 | 15.81 | 13.31 |
| 239 | THR | C | 19.37 | 48.89 | 15.62 | 13.46 |
| 239 | THR | O | 20.14 | 48.22 | 14.94 | 13.46 |
| 240 | LYS | N | 19.77 | 49.66 | 16.62 | 13.89 |
| 240 | LYS | CA | 21.18 | 49.78 | 16.98 | 14.34 |
| 240 | LYS | CB | 21.34 | 50.83 | 18.09 | 14.50 |
| 240 | LYS | CG | 20.42 | 50.63 | 19.29 | 14.82 |
| 240 | LYS | CD | 20.43 | 51.86 | 20.21 | 15.14 |
| 240 | LYS | CE | 19.52 | 51.66 | 21.42 | 15.34 |
| 240 | LYS | NZ | 18.12 | 51.27 | 21.03 | 15.57 |
| 240 | LYS | C | 21.76 | 48.45 | 17.44 | 14.50 |
| 240 | LYS | O | 22.98 | 48.24 | 17.38 | 14.60 |
| 241 | ASP | N | 20.89 | 47.55 | 17.89 | 14.69 |
| 241 | ASP | CA | 21.33 | 46.25 | 18.38 | 14.83 |
| 241 | ASP | CB | 20.56 | 45.89 | 19.65 | 15.16 |
| 241 | ASP | CG | 20.86 | 46.83 | 20.80 | 15.41 |
| 241 | ASP | OD1 | 22.05 | 47.14 | 21.00 | 15.60 |
| 241 | ASP | OD2 | 19.91 | 47.26 | 21.48 | 15.65 |
| 241 | ASP | C | 21.18 | 45.14 | 17.35 | 14.72 |
| 241 | ASP | O | 21.29 | 43.95 | 17.68 | 14.88 |
| 242 | GLY | N | 20.95 | 45.52 | 16.10 | 14.47 |
| 242 | GLY | CA | 20.79 | 44.53 | 15.06 | 14.18 |
| 242 | GLY | C | 19.38 | 44.53 | 14.49 | 13.89 |
| 242 | GLY | O | 18.43 | 44.94 | 15.17 | 13.91 |
| 243 | PRO | N | 19.21 | 44.07 | 13.24 | 13.59 |
| 243 | PRO | CD | 20.24 | 43.60 | 12.31 | 13.53 |
| 243 | PRO | CA | 17.89 | 44.03 | 12.62 | 13.37 |
| 243 | PRO | CB | 18.20 | 43.65 | 11.18 | 13.32 |
| 243 | PRO | CG | 19.43 | 42.81 | 11.31 | 13.44 |
| 243 | PRO | C | 16.91 | 43.07 | 13.28 | 13.02 |
| 243 | PRO | O | 17.28 | 41.98 | 13.73 | 13.03 |
| 244 | ILE | N | 15.65 | 43.48 | 13.34 | 12.67 |
| 244 | ILE | CA | 14.60 | 42.67 | 13.91 | 12.43 |
| 244 | ILE | CB | 13.73 | 43.48 | 14.89 | 12.43 |
| 244 | ILE | CG2 | 12.57 | 42.64 | 15.38 | 12.42 |
| 244 | ILE | CG1 | 14.58 | 43.98 | 16.05 | 12.52 |
| 244 | ILE | CD1 | 13.78 | 44.68 | 17.14 | 12.70 |
| 244 | ILE | C | 13.71 | 42.20 | 12.77 | 12.09 |
| 244 | ILE | O | 13.16 | 43.02 | 12.04 | 12.05 |
| 245 | LEU | N | 13.60 | 40.89 | 12.60 | 11.88 |
| 245 | LEU | CA | 12.74 | 40.35 | 11.55 | 11.62 |
| 245 | LEU | CB | 13.35 | 39.08 | 10.95 | 11.62 |
| 245 | LEU | CG | 14.42 | 39.27 | 9.87 | 11.75 |
| 245 | LEU | CD1 | 15.54 | 40.16 | 10.39 | 11.78 |
| 245 | LEU | CD2 | 14.97 | 37.91 | 9.46 | 11.77 |
| 245 | LEU | C | 11.39 | 40.02 | 12.18 | 11.39 |
| 245 | LEU | O | 11.27 | 39.05 | 12.93 | 11.43 |
| 246 | THR | N | 10.38 | 40.83 | 11.88 | 11.22 |
| 246 | THR | CA | 9.06 | 40.59 | 12.45 | 11.08 |
| 246 | THR | CB | 8.13 | 41.80 | 12.23 | 10.99 |
| 246 | THR | OG1 | 7.85 | 41.93 | 10.84 | 10.89 |
| 246 | THR | CG2 | 8.80 | 43.09 | 12.72 | 10.94 |
| 246 | THR | C | 8.41 | 39.36 | 11.85 | 10.92 |
| 246 | THR | O | 7.53 | 38.76 | 12.46 | 10.84 |
| 247 | THR | N | 8.87 | 38.96 | 10.66 | 11.03 |
| 247 | THR | CA | 8.30 | 37.81 | 9.96 | 11.10 |
| 247 | THR | CB | 8.09 | 38.14 | 8.48 | 10.97 |
| 247 | THR | OG1 | 9.31 | 38.63 | 7.92 | 10.93 |
| 247 | THR | CG2 | 7.00 | 39.20 | 8.32 | 10.94 |
| 247 | THR | C | 9.11 | 36.52 | 10.08 | 11.25 |
| 247 | THR | O | 8.92 | 35.59 | 9.29 | 11.08 |
| 248 | LYS | N | 10.00 | 36.48 | 11.06 | 11.62 |
| 248 | LYS | CA | 10.81 | 35.29 | 11.30 | 12.10 |
| 248 | LYS | CB | 12.30 | 35.65 | 11.38 | 12.27 |
| 248 | LYS | CG | 13.18 | 34.46 | 11.71 | 12.62 |
| 248 | LYS | CD | 14.62 | 34.87 | 11.96 | 13.00 |
| 248 | LYS | CE | 15.45 | 33.67 | 12.36 | 13.30 |
| 248 | LYS | NZ | 16.85 | 34.08 | 12.68 | 13.72 |
| 248 | LYS | C | 10.36 | 34.70 | 12.62 | 12.35 |
| 248 | LYS | O | 10.33 | 35.39 | 13.64 | 12.36 |
| 249 | ILE | N | 9.98 | 33.42 | 12.60 | 12.68 |
| 249 | ILE | CA | 9.54 | 32.72 | 13.80 | 13.06 |
| 249 | ILE | CB | 8.29 | 31.85 | 13.52 | 12.88 |
| 249 | ILE | CG2 | 8.01 | 30.93 | 14.71 | 12.97 |
| 249 | ILE | CG1 | 7.08 | 32.73 | 13.25 | 12.80 |
| 249 | ILE | CD1 | 5.84 | 31.95 | 12.85 | 12.43 |
| 249 | ILE | C | 10.67 | 31.81 | 14.27 | 13.35 |
| 249 | ILE | O | 11.13 | 30.99 | 13.45 | 13.62 |
| 249 | ILE | OT | 11.07 | 31.93 | 15.45 | 13.77 |
| 301 | HOH | O | 10.52 | 34.41 | 4.34 | 9.04 |
| 302 | HOH | O | 5.68 | 43.84 | 10.75 | 7.39 |
| 303 | HOH | O | −1.54 | 38.06 | 1.99 | 12.17 |
| 304 | HOH | O | 9.58 | 31.37 | −7.05 | 11.42 |
| 305 | HOH | O | 3.84 | 35.16 | −5.21 | 13.13 |
| 306 | HOH | O | 15.54 | 21.70 | −8.21 | 11.28 |
| 307 | HOH | O | 1.27 | 24.06 | −11.29 | 10.96 |
| 308 | HOH | O | 2.83 | 15.97 | −9.91 | 13.21 |
| 309 | HOH | O | 7.75 | 29.41 | 5.29 | 11.48 |
| 310 | HOH | O | 0.00 | 23.10 | 6.79 | 13.97 |
| 311 | HOH | O | 15.64 | 45.67 | −8.01 | 12.06 |
| 312 | HOH | O | 9.80 | 28.82 | −15.22 | 13.56 |
| 313 | HOH | O | 17.62 | 39.52 | −19.14 | 15.74 |
| 314 | HOH | O | −0.83 | 44.37 | 14.52 | 11.62 |
| 315 | HOH | O | −0.42 | 38.31 | 20.32 | 14.43 |
| 316 | HOH | O | −1.83 | 34.36 | −12.10 | 11.01 |
| 317 | HOH | O | 19.18 | 48.28 | −2.44 | 14.08 |
| 318 | HOH | O | −0.15 | 30.63 | −9.41 | 14.58 |
| 319 | HOH | O | 1.31 | 39.36 | −0.48 | 15.80 |
| 320 | HOH | O | 17.43 | 50.70 | −3.01 | 11.91 |
| 321 | HOH | O | 16.96 | 43.75 | −2.50 | 15.47 |
| 322 | HOH | O | 27.70 | 32.63 | −2.22 | 17.18 |
| 323 | HOH | O | 7.13 | 38.87 | 15.05 | 13.04 |
| 324 | HOH | O | 6.69 | 25.10 | 6.71 | 15.96 |
| 325 | HOH | O | 12.13 | 26.59 | 2.42 | 16.87 |
| 326 | HOH | O | 9.11 | 23.19 | 1.27 | 16.15 |
| 327 | HOH | O | 12.01 | 32.52 | 8.22 | 15.82 |
| 328 | HOH | O | −10.14 | 24.71 | 0.78 | 18.07 |
| 329 | HOH | O | 10.19 | 30.74 | −11.11 | 12.76 |
| 330 | HOH | O | 27.52 | 34.99 | −0.31 | 22.73 |
| 331 | HOH | O | 25.00 | 45.61 | −8.38 | 15.19 |
| 332 | HOH | O | −1.54 | 30.46 | −12.60 | 15.24 |
| 333 | HOH | O | 19.47 | 56.55 | 13.08 | 16.06 |
| 334 | HOH | O | −8.38 | 38.74 | 4.73 | 12.15 |
| 335 | HOH | O | −6.73 | 18.96 | −3.18 | 15.71 |
| 336 | HOH | O | −10.97 | 34.30 | 0.08 | 13.27 |
| 337 | HOH | O | 21.12 | 50.41 | −4.73 | 16.42 |
| 338 | HOH | O | 21.28 | 28.77 | 1.36 | 19.93 |
| 339 | HOH | O | 21.90 | 54.50 | −0.49 | 15.36 |
| 340 | HOH | O | 6.31 | 38.98 | −4.46 | 15.11 |
| 341 | HOH | O | 0.28 | 47.87 | 20.07 | 17.85 |
| 342 | HOH | O | 10.30 | 52.79 | 6.53 | 13.97 |
| 343 | HOH | O | 21.05 | 22.71 | −6.43 | 18.60 |
| 344 | HOH | O | 24.76 | 54.33 | −3.46 | 17.89 |
| 345 | HOH | O | 8.27 | 55.29 | 6.13 | 17.47 |
| 346 | HOH | O | 14.63 | 39.13 | 14.67 | 17.13 |
| 347 | HOH | O | 26.93 | 45.54 | 0.58 | 16.06 |
| 348 | HOH | O | 22.00 | 40.33 | −12.38 | 18.56 |
| 349 | HOH | O | 22.94 | 37.71 | 8.22 | 17.56 |
| 350 | HOH | O | −9.57 | 25.12 | −6.14 | 17.84 |
| 351 | HOH | O | 14.01 | 37.19 | −21.87 | 21.98 |
| 352 | HOH | O | 4.97 | 28.02 | 11.97 | 18.66 |
| 353 | HOH | O | −5.14 | 33.79 | −15.46 | 19.79 |
| 354 | HOH | O | 0.89 | 27.80 | −16.88 | 20.55 |
| 355 | HOH | O | 0.04 | 44.37 | 19.31 | 19.74 |
| 356 | HOH | O | −3.82 | 23.00 | −13.39 | 19.63 |
| 357 | HOH | O | −5.33 | 28.61 | −13.52 | 20.29 |
| 358 | HOH | O | −1.83 | 19.71 | 1.80 | 18.07 |
| 359 | HOH | O | 6.32 | 20.97 | −0.93 | 18.32 |
| 360 | HOH | O | 27.95 | 42.43 | −8.15 | 16.27 |
| 361 | HOH | O | −12.42 | 27.09 | −2.81 | 19.70 |
| 362 | HOH | O | 6.92 | 56.74 | 10.12 | 19.28 |
| 363 | HOH | O | 4.38 | 48.07 | 26.38 | 17.88 |
| 364 | HOH | O | 19.09 | 59.51 | 4.00 | 22.51 |
| 365 | HOH | O | 0.29 | 20.44 | 5.95 | 21.11 |
| 366 | HOH | O | 22.32 | 49.11 | 4.73 | 22.73 |
| 367 | HOH | O | −7.96 | 33.86 | −5.80 | 15.94 |
| 368 | HOH | O | 10.38 | 12.96 | −7.37 | 19.87 |
| 369 | HOH | O | 10.83 | 20.64 | −13.14 | 20.14 |
| 370 | HOH | O | 14.02 | 19.19 | −4.63 | 17.34 |
| 371 | HOH | O | 27.65 | 28.59 | −6.88 | 22.89 |
| 372 | HOH | O | −6.46 | 47.08 | 10.42 | 18.12 |
| 373 | HOH | O | −10.07 | 39.53 | 10.48 | 21.30 |
| 374 | HOH | O | 26.55 | 63.94 | 0.02 | 19.49 |
| 375 | HOH | O | 3.08 | 24.90 | 9.68 | 23.60 |
| 376 | HOH | O | −2.68 | 51.64 | 4.49 | 18.77 |
| 377 | HOH | O | 13.98 | 54.38 | −2.78 | 20.57 |
| 378 | HOH | O | 6.01 | 26.98 | −17.07 | 18.20 |
| 379 | HOH | O | 10.76 | 28.22 | 8.28 | 21.31 |
| 380 | HOH | O | 7.21 | 21.14 | −12.51 | 22.12 |
| 381 | HOH | O | −0.01 | 47.70 | 24.74 | 20.57 |
| 382 | HOH | O | 26.14 | 36.21 | −13.19 | 18.02 |
| 383 | HOH | O | 8.71 | 24.35 | −14.71 | 17.36 |
| 384 | HOH | O | −1.02 | 26.70 | −15.46 | 21.86 |
| 385 | HOH | O | 11.63 | 19.79 | −2.08 | 20.87 |
| 386 | HOH | O | 24.46 | 45.23 | 4.56 | 21.68 |
| 387 | HOH | O | −10.35 | 35.86 | 6.34 | 17.79 |
| 388 | HOH | O | 9.16 | 13.56 | −4.09 | 24.33 |
| 389 | HOH | O | 14.88 | 29.32 | 2.52 | 23.60 |
| 390 | HOH | O | 7.93 | 59.35 | 12.82 | 21.92 |
| 391 | HOH | O | −0.39 | 23.55 | 11.16 | 24.02 |
| 392 | HOH | O | −12.19 | 39.32 | 7.34 | 20.29 |
| 393 | HOH | O | 4.62 | 40.47 | −21.40 | 20.86 |
| 394 | HOH | O | 10.83 | 29.71 | 6.02 | 18.68 |
| 395 | HOH | O | 24.32 | 29.38 | −12.36 | 22.37 |
| 396 | HOH | O | 28.11 | 43.37 | −0.40 | 21.07 |
| 397 | HOH | O | −6.47 | 51.51 | 11.11 | 21.48 |
| 398 | HOH | O | −13.50 | 29.57 | 1.74 | 21.65 |
| 399 | HOH | O | 19.82 | 47.00 | 12.43 | 19.88 |
| 400 | HOH | O | 4.96 | 19.65 | 5.04 | 23.26 |
| 401 | HOH | O | 20.66 | 22.51 | −14.45 | 21.14 |
| 402 | HOH | O | 23.27 | 50.91 | −11.12 | 19.97 |
| 403 | HOH | O | 24.73 | 27.34 | −2.60 | 22.99 |
| 404 | HOH | O | 10.48 | 25.34 | −17.84 | 20.43 |
| 405 | HOH | O | 12.29 | 57.56 | 5.75 | 21.45 |
| 406 | HOH | O | 4.36 | 24.78 | −15.46 | 23.33 |
| 407 | HOH | O | 27.81 | 38.95 | 4.53 | 25.57 |
| 408 | HOH | O | 16.80 | 29.38 | −17.21 | 18.25 |
| 409 | HOH | O | 11.24 | 56.92 | −1.08 | 23.15 |
| 410 | HOH | O | 27.14 | 41.96 | 3.63 | 19.44 |
| 411 | HOH | O | 8.80 | 44.92 | −17.88 | 21.62 |
| 412 | HOH | O | 7.99 | 47.13 | −4.60 | 21.32 |
| 413 | HOH | O | 4.03 | 21.88 | −13.51 | 21.83 |
| 414 | HOH | O | 10.11 | 30.56 | −23.57 | 19.04 |
| 415 | HOH | O | 2.49 | 20.92 | 9.21 | 23.07 |
| 416 | HOH | O | −3.68 | 18.23 | −0.95 | 23.22 |
| 417 | HOH | O | 2.21 | 18.16 | 2.62 | 24.12 |
| 418 | HOH | O | −7.20 | 45.05 | 12.15 | 17.72 |
| 419 | HOH | O | 3.33 | 30.32 | −20.15 | 23.20 |
| 420 | HOH | O | −10.10 | 21.91 | −7.96 | 24.45 |
| 421 | HOH | O | 6.20 | 32.93 | −19.28 | 20.30 |
| 422 | HOH | O | 26.47 | 50.59 | −1.04 | 20.30 |
| 423 | HOH | O | 4.73 | 12.67 | −7.41 | 20.45 |
| 424 | HOH | O | 30.96 | 47.29 | 7.54 | 23.26 |
| 425 | HOH | O | 5.48 | 21.45 | 8.98 | 23.62 |
| 426 | HOH | O | 7.63 | 53.70 | 24.03 | 20.67 |
| 427 | HOH | O | 24.84 | 33.85 | 3.40 | 19.47 |
| 428 | HOH | O | −6.35 | 22.25 | 2.44 | 21.89 |
| 429 | HOH | O | 14.70 | 22.58 | −16.76 | 21.77 |
| 430 | HOH | O | 3.75 | 54.62 | −2.08 | 23.59 |
| 431 | HOH | O | 22.02 | 24.22 | −11.35 | 20.43 |
| 432 | HOH | O | −3.17 | 29.64 | −17.10 | 24.33 |
| 433 | HOH | O | 13.43 | 57.79 | 1.30 | 20.34 |
| 434 | HOH | O | 12.73 | 49.05 | 18.00 | 22.94 |
| 435 | HOH | O | −1.86 | 47.35 | 18.38 | 20.80 |
| 436 | HOH | O | −8.80 | 32.90 | −9.68 | 22.48 |
| 437 | HOH | O | 7.68 | 56.75 | −0.80 | 23.53 |
| 438 | HOH | O | 2.12 | 54.11 | 19.73 | 20.27 |
| 439 | HOH | O | 20.02 | 27.96 | 5.22 | 22.64 |
| 440 | HOH | O | 27.87 | 54.67 | −2.71 | 21.05 |
| 441 | HOH | O | 5.68 | 17.32 | −1.75 | 21.40 |
| 442 | HOH | O | −2.40 | 21.46 | 9.46 | 25.37 |
| 443 | HOH | O | 14.70 | 33.35 | 6.08 | 20.03 |
| 444 | HOH | O | 9.71 | 29.38 | 10.63 | 15.52 |
| 445 | HOH | O | 24.87 | 51.89 | 13.40 | 19.22 |
| 446 | HOH | O | 26.44 | 38.92 | −13.22 | 22.46 |
| 447 | HOH | O | 25.13 | 34.40 | 7.66 | 23.31 |
| 448 | HOH | O | 26.10 | 25.40 | −8.41 | 22.30 |
| 449 | HOH | O | 25.14 | 25.44 | −5.06 | 23.19 |
| 450 | HOH | O | 8.38 | 17.72 | −15.21 | 23.09 |
| 451 | HOH | O | 22.19 | 29.97 | −15.30 | 23.57 |
| 452 | HOH | O | 18.33 | 34.74 | −23.33 | 24.25 |
| 453 | HOH | O | 10.42 | 50.74 | 19.99 | 23.68 |
| 454 | HOH | O | 3.82 | 37.92 | −5.15 | 11.14 |
| 455 | HOH | O | 2.27 | 13.52 | −8.36 | 12.29 |
| 456 | HOH | O | −2.78 | 30.00 | −9.48 | 13.74 |
| 457 | HOH | O | −9.55 | 38.20 | 7.08 | 17.08 |
| 458 | HOH | O | −3.75 | 32.18 | −11.75 | 14.53 |
| 459 | HOH | O | 19.30 | 52.33 | −4.24 | 16.15 |
| 460 | HOH | O | 13.16 | 27.86 | 0.31 | 14.90 |
| 461 | HOH | O | 8.48 | 26.74 | −16.17 | 16.16 |
| 462 | HOH | O | −9.40 | 32.09 | −0.23 | 14.19 |
| 463 | HOH | O | 14.64 | 44.03 | −1.79 | 12.77 |
| 464 | HOH | O | 26.82 | 44.37 | −6.48 | 10.19 |
| 465 | HOH | O | −1.79 | 44.63 | 17.17 | 17.83 |
| 466 | HOH | O | 9.95 | 34.41 | −7.10 | 16.75 |
| 467 | HOH | O | 1.11 | 22.95 | 9.12 | 20.95 |
| 468 | HOH | O | 12.16 | 38.23 | −18.69 | 15.18 |
| 469 | HOH | O | 7.37 | 23.04 | 5.01 | 18.28 |
| 470 | HOH | O | −11.15 | 38.22 | 12.41 | 20.33 |
| 471 | HOH | O | 28.61 | 30.24 | −0.81 | 17.78 |
| 472 | HOH | O | −0.96 | 26.32 | 11.35 | 20.16 |
| 473 | HOH | O | 24.13 | 38.95 | −11.62 | 18.68 |
| 474 | HOH | O | 4.45 | 14.71 | −12.00 | 19.97 |
| 475 | HOH | O | −0.69 | 18.90 | 4.10 | 19.92 |
| 476 | HOH | O | 6.05 | 24.26 | 8.94 | 23.08 |
| 477 | HOH | O | −8.40 | 22.26 | −10.37 | 18.50 |
| 478 | HOH | O | −3.90 | 21.03 | 3.27 | 19.43 |
| 479 | HOH | O | −9.00 | 28.94 | −12.03 | 19.69 |
| 480 | HOH | O | 5.72 | 43.88 | −19.58 | 21.53 |
| 481 | HOH | O | −10.34 | 40.76 | 3.80 | 19.91 |
| 482 | HOH | O | 4.65 | 19.40 | −14.69 | 21.72 |
| 483 | HOH | O | 14.18 | 31.10 | 4.58 | 21.73 |
| 484 | HOH | O | 30.32 | 41.65 | −7.82 | 19.22 |
| 485 | HOH | O | 21.32 | 35.97 | 9.01 | 20.31 |
| 486 | HOH | O | 4.48 | 29.50 | 16.49 | 20.31 |
| 487 | HOH | O | 29.18 | 37.40 | 0.15 | 19.97 |
| 488 | HOH | O | 20.08 | 54.41 | 17.08 | 21.63 |
| 489 | HOH | O | 15.85 | 30.46 | −19.41 | 20.67 |
| 490 | HOH | O | 20.53 | 60.95 | 2.00 | 16.09 |
| 491 | HOH | O | 23.10 | 22.71 | −8.27 | 21.27 |
| 492 | HOH | O | −4.11 | 51.39 | −1.95 | 22.75 |
| 493 | HOH | O | −11.18 | 28.95 | 3.76 | 20.88 |
| 494 | HOH | O | 7.02 | 22.64 | −15.50 | 20.80 |
| 253 | ZN2 | ZN | 7.01 | 43.39 | −0.45 | 14.29 |
| 254 | ZN2 | ZN | 5.57 | 40.37 | −0.35 | 13.09 |

TABLE II
Provides a three dimensional protein coordinate set of
a S. aureus methionine aminopeptidase crystalline structure
of an inhibitor complex with 5-(3-Iodo-phenyl)-1-H-[1,2,3]triazole.

| Residue Atom | | | X | Y | Z | B |
|---|---|---|---|---|---|---|
| 1 | MET | CB | 6.21 | 53.26 | 9.50 | 19.23 |
| 1 | MET | CG | 6.45 | 52.04 | 10.38 | 19.33 |
| 1 | MET | SD | 5.09 | 51.62 | 11.50 | 19.46 |
| 1 | MET | CE | 5.33 | 52.85 | 12.79 | 19.56 |
| 1 | MET | C | 8.65 | 53.68 | 9.58 | 19.14 |
| 1 | MET | O | 9.57 | 52.88 | 9.43 | 19.09 |
| 1 | MET | N | 7.67 | 52.67 | 7.58 | 19.08 |
| 1 | MET | CA | 7.43 | 53.66 | 8.67 | 19.14 |
| 2 | ILE | N | 8.64 | 54.61 | 10.53 | 19.17 |
| 2 | ILE | CA | 9.72 | 54.73 | 11.50 | 19.25 |
| 2 | ILE | CB | 10.30 | 56.16 | 11.56 | 19.26 |
| 2 | ILE | CG2 | 11.43 | 56.21 | 12.59 | 19.29 |
| 2 | ILE | CG1 | 10.81 | 56.58 | 10.18 | 19.29 |
| 2 | ILE | CD1 | 11.96 | 55.74 | 9.67 | 19.29 |
| 2 | ILE | C | 9.14 | 54.40 | 12.87 | 19.27 |
| 2 | ILE | O | 8.24 | 55.10 | 13.34 | 19.28 |
| 3 | VAL | N | 9.63 | 53.34 | 13.49 | 19.35 |
| 3 | VAL | CA | 9.14 | 52.94 | 14.81 | 19.44 |
| 3 | VAL | CB | 9.62 | 51.52 | 15.18 | 19.43 |
| 3 | VAL | CG1 | 9.22 | 51.19 | 16.61 | 19.46 |
| 3 | VAL | CG2 | 9.04 | 50.51 | 14.21 | 19.46 |
| 3 | VAL | C | 9.63 | 53.91 | 15.88 | 19.52 |
| 3 | VAL | O | 10.82 | 54.15 | 16.01 | 19.54 |
| 4 | LYS | N | 8.69 | 54.46 | 16.64 | 19.62 |
| 4 | LYS | CA | 9.03 | 55.40 | 17.70 | 19.71 |
| 4 | LYS | CB | 8.34 | 56.75 | 17.45 | 19.88 |
| 4 | LYS | CG | 8.73 | 57.46 | 16.16 | 20.17 |
| 4 | LYS | CD | 10.09 | 58.16 | 16.26 | 20.41 |
| 4 | LYS | CE | 11.25 | 57.20 | 16.09 | 20.58 |
| 4 | LYS | NZ | 12.56 | 57.91 | 16.16 | 20.77 |
| 4 | LYS | C | 8.60 | 54.88 | 19.07 | 19.68 |
| 4 | LYS | O | 9.24 | 55.18 | 20.08 | 19.72 |
| 5 | THR | N | 7.53 | 54.09 | 19.11 | 19.59 |
| 5 | THR | CA | 7.03 | 53.57 | 20.38 | 19.50 |
| 5 | THR | CB | 5.61 | 54.09 | 20.67 | 19.53 |
| 5 | THR | OG1 | 4.67 | 53.41 | 19.82 | 19.54 |
| 5 | THR | CG2 | 5.52 | 55.59 | 20.39 | 19.53 |
| 5 | THR | C | 6.98 | 52.05 | 20.52 | 19.43 |
| 5 | THR | O | 6.99 | 51.32 | 19.53 | 19.44 |
| 6 | GLU | N | 6.91 | 51.60 | 21.76 | 19.31 |
| 6 | GLU | CA | 6.84 | 50.18 | 22.09 | 19.16 |
| 6 | GLU | CB | 6.88 | 50.01 | 23.62 | 19.39 |
| 6 | GLU | CG | 6.60 | 48.60 | 24.14 | 19.71 |
| 6 | GLU | CD | 7.64 | 47.58 | 23.73 | 19.87 |
| 6 | GLU | OE1 | 8.83 | 47.95 | 23.63 | 20.04 |
| 6 | GLU | OE2 | 7.28 | 46.40 | 23.53 | 20.01 |
| 6 | GLU | C | 5.55 | 49.59 | 21.53 | 18.92 |
| 6 | GLU | O | 5.53 | 48.45 | 21.07 | 18.90 |
| 7 | GLU | N | 4.47 | 50.37 | 21.57 | 18.58 |
| 7 | GLU | CA | 3.18 | 49.92 | 21.06 | 18.24 |
| 7 | GLU | CB | 2.10 | 50.97 | 21.36 | 18.47 |
| 7 | GLU | CG | 1.70 | 51.09 | 22.83 | 18.77 |
| 7 | GLU | CD | 2.82 | 51.62 | 23.71 | 18.95 |
| 7 | GLU | OE1 | 3.53 | 52.57 | 23.31 | 19.05 |
| 7 | GLU | OE2 | 2.98 | 51.09 | 24.84 | 19.23 |
| 7 | GLU | C | 3.24 | 49.66 | 19.55 | 17.81 |
| 7 | GLU | O | 2.63 | 48.72 | 19.05 | 17.76 |
| 8 | GLU | N | 3.97 | 50.50 | 18.83 | 17.30 |
| 8 | GLU | CA | 4.10 | 50.33 | 17.39 | 16.78 |
| 8 | GLU | CB | 4.81 | 51.53 | 16.76 | 16.90 |
| 8 | GLU | CG | 4.07 | 52.84 | 16.93 | 17.05 |
| 8 | GLU | CD | 4.83 | 54.03 | 16.36 | 17.13 |
| 8 | GLU | OE1 | 6.07 | 54.05 | 16.49 | 17.26 |
| 8 | GLU | OE2 | 4.18 | 54.95 | 15.81 | 17.30 |
| 8 | GLU | C | 4.90 | 49.06 | 17.12 | 16.32 |
| 8 | GLU | O | 4.57 | 48.27 | 16.23 | 16.25 |
| 9 | LEU | N | 5.95 | 48.86 | 17.91 | 15.83 |
| 9 | LEU | CA | 6.80 | 47.68 | 17.78 | 15.30 |
| 9 | LEU | CB | 7.94 | 47.73 | 18.80 | 15.39 |
| 9 | LEU | CG | 8.87 | 46.51 | 18.84 | 15.42 |
| 9 | LEU | CD1 | 9.53 | 46.28 | 17.49 | 15.48 |
| 9 | LEU | CD2 | 9.92 | 46.71 | 19.92 | 15.51 |
| 9 | LEU | C | 5.97 | 46.41 | 17.98 | 14.95 |
| 9 | LEU | O | 6.10 | 45.46 | 17.22 | 14.78 |
| 10 | GLN | N | 5.14 | 46.42 | 19.02 | 14.50 |
| 10 | GLN | CA | 4.30 | 45.27 | 19.32 | 14.04 |
| 10 | GLN | CB | 3.58 | 45.49 | 20.65 | 14.12 |
| 10 | GLN | CG | 4.53 | 45.54 | 21.84 | 14.31 |
| 10 | GLN | CD | 3.84 | 45.99 | 23.11 | 14.39 |
| 10 | GLN | OE1 | 2.64 | 46.24 | 23.13 | 14.55 |
| 10 | GLN | NE2 | 4.62 | 46.09 | 24.19 | 14.46 |
| 10 | GLN | C | 3.29 | 45.01 | 18.20 | 13.72 |
| 10 | GLN | O | 3.04 | 43.86 | 17.84 | 13.59 |
| 11 | ALA | N | 2.71 | 46.07 | 17.66 | 13.26 |
| 11 | ALA | CA | 1.75 | 45.92 | 16.59 | 12.85 |
| 11 | ALA | CB | 1.16 | 47.27 | 16.22 | 12.91 |
| 11 | ALA | C | 2.42 | 45.28 | 15.37 | 12.60 |
| 11 | ALA | O | 1.85 | 44.41 | 14.72 | 12.49 |
| 12 | LEU | N | 3.64 | 45.72 | 15.07 | 12.27 |
| 12 | LEU | CA | 4.38 | 45.18 | 13.92 | 11.98 |
| 12 | LEU | CB | 5.63 | 46.03 | 13.65 | 12.00 |
| 12 | LEU | CG | 5.32 | 47.44 | 13.14 | 12.01 |
| 12 | LEU | CD1 | 6.56 | 48.31 | 13.23 | 12.05 |
| 12 | LEU | CD2 | 4.82 | 47.36 | 11.70 | 12.04 |
| 12 | LEU | C | 4.77 | 43.72 | 14.15 | 11.78 |
| 12 | LEU | O | 4.73 | 42.90 | 13.23 | 11.73 |
| 13 | LYS | N | 5.14 | 43.40 | 15.38 | 11.61 |
| 13 | LYS | CA | 5.52 | 42.03 | 15.71 | 11.46 |
| 13 | LYS | CB | 6.13 | 41.96 | 17.11 | 11.73 |
| 13 | LYS | CG | 7.51 | 42.58 | 17.20 | 12.09 |
| 13 | LYS | CD | 8.05 | 42.46 | 18.61 | 12.47 |
| 13 | LYS | CE | 9.50 | 42.88 | 18.69 | 12.78 |
| 13 | LYS | NZ | 10.04 | 42.64 | 20.06 | 13.07 |
| 13 | LYS | C | 4.31 | 41.11 | 15.62 | 11.20 |
| 13 | LYS | O | 4.42 | 39.95 | 15.21 | 11.18 |
| 14 | GLU | N | 3.15 | 41.63 | 16.01 | 10.92 |
| 14 | GLU | CA | 1.91 | 40.85 | 15.97 | 10.59 |
| 14 | GLU | CB | 0.75 | 41.67 | 16.56 | 10.78 |
| 14 | GLU | CG | −0.64 | 41.05 | 16.39 | 10.96 |
| 14 | GLU | CD | −0.84 | 39.79 | 17.21 | 11.11 |
| 14 | GLU | OE1 | 0.06 | 39.40 | 17.98 | 11.34 |
| 14 | GLU | OE2 | −1.93 | 39.18 | 17.08 | 11.36 |
| 14 | GLU | C | 1.56 | 40.45 | 14.54 | 10.33 |
| 14 | GLU | O | 1.42 | 39.26 | 14.24 | 10.20 |
| 15 | ILE | N | 1.44 | 41.43 | 13.65 | 10.01 |
| 15 | ILE | CA | 1.09 | 41.13 | 12.26 | 9.77 |
| 15 | ILE | CB | 0.70 | 42.44 | 11.50 | 9.70 |
| 15 | ILE | CG2 | 1.89 | 43.38 | 11.41 | 9.67 |
| 15 | ILE | CG1 | 0.17 | 42.10 | 10.10 | 9.57 |
| 15 | ILE | CD1 | −1.07 | 41.23 | 10.11 | 9.36 |
| 15 | ILE | C | 2.21 | 40.37 | 11.54 | 9.65 |
| 15 | ILE | O | 1.95 | 39.58 | 10.63 | 9.68 |
| 16 | GLY | N | 3.46 | 40.59 | 11.97 | 9.53 |
| 16 | GLY | CA | 4.57 | 39.87 | 11.37 | 9.25 |
| 16 | GLY | C | 4.47 | 38.39 | 11.71 | 9.14 |
| 16 | GLY | O | 4.71 | 37.53 | 10.86 | 8.92 |
| 17 | TYR | N | 4.13 | 38.09 | 12.96 | 9.12 |
| 17 | TYR | CA | 3.99 | 36.70 | 13.38 | 9.13 |
| 17 | TYR | CB | 3.66 | 36.61 | 14.88 | 9.34 |
| 17 | TYR | CG | 3.26 | 35.22 | 15.29 | 9.68 |
| 17 | TYR | CD1 | 4.21 | 34.22 | 15.46 | 9.90 |
| 17 | TYR | CE1 | 3.83 | 32.91 | 15.74 | 10.16 |
| 17 | TYR | CD2 | 1.91 | 34.87 | 15.40 | 9.92 |
| 17 | TYR | CE2 | 1.51 | 33.56 | 15.68 | 10.14 |
| 17 | TYR | CZ | 2.49 | 32.58 | 15.84 | 10.18 |
| 17 | TYR | OH | 2.12 | 31.27 | 16.08 | 10.55 |
| 17 | TYR | C | 2.87 | 36.03 | 12.60 | 8.95 |
| 17 | TYR | O | 3.02 | 34.90 | 12.12 | 8.87 |
| 18 | ILE | N | 1.74 | 36.73 | 12.47 | 8.81 |
| 18 | ILE | CA | 0.60 | 36.20 | 11.74 | 8.65 |
| 18 | ILE | CB | −0.57 | 37.22 | 11.76 | 8.59 |
| 18 | ILE | CG2 | −1.68 | 36.77 | 10.81 | 8.56 |
| 18 | ILE | CG1 | −1.11 | 37.35 | 13.19 | 8.62 |
| 18 | ILE | CD1 | −2.17 | 38.43 | 13.36 | 8.71 |
| 18 | ILE | C | 0.97 | 35.87 | 10.30 | 8.59 |
| 18 | ILE | O | 0.64 | 34.80 | 9.79 | 8.55 |
| 19 | CYS | N | 1.69 | 36.78 | 9.65 | 8.55 |
| 19 | CYS | CA | 2.07 | 36.52 | 8.27 | 8.47 |
| 19 | CYS | CB | 2.67 | 37.78 | 7.63 | 8.47 |
| 19 | CYS | SG | 1.39 | 39.01 | 7.25 | 8.50 |
| 19 | CYS | C | 3.04 | 35.35 | 8.16 | 8.50 |
| 19 | CYS | O | 2.96 | 34.55 | 7.22 | 8.51 |
| 20 | ALA | N | 3.96 | 35.24 | 9.13 | 8.51 |
| 20 | ALA | CA | 4.92 | 34.14 | 9.13 | 8.54 |
| 20 | ALA | CB | 5.96 | 34.38 | 10.23 | 8.61 |
| 20 | ALA | C | 4.20 | 32.81 | 9.36 | 8.66 |
| 20 | ALA | O | 4.50 | 31.81 | 8.71 | 8.52 |
| 21 | LYS | N | 3.25 | 32.82 | 10.28 | 8.77 |
| 21 | LYS | CA | 2.48 | 31.61 | 10.59 | 9.01 |
| 21 | LYS | CB | 1.50 | 31.90 | 11.73 | 9.34 |
| 21 | LYS | CG | 0.59 | 30.73 | 12.11 | 10.01 |
| 21 | LYS | CD | 1.38 | 29.55 | 12.63 | 10.53 |
| 21 | LYS | CE | 0.44 | 28.50 | 13.22 | 10.90 |
| 21 | LYS | NZ | 1.13 | 27.23 | 13.58 | 11.29 |
| 21 | LYS | C | 1.73 | 31.15 | 9.35 | 8.95 |
| 21 | LYS | O | 1.70 | 29.96 | 9.02 | 9.02 |
| 22 | VAL | N | 1.11 | 32.10 | 8.65 | 8.94 |
| 22 | VAL | CA | 0.36 | 31.78 | 7.44 | 8.95 |
| 22 | VAL | CB | −0.40 | 33.03 | 6.93 | 8.97 |
| 22 | VAL | CG1 | −0.97 | 32.79 | 5.53 | 8.97 |
| 22 | VAL | CG2 | −1.53 | 33.35 | 7.89 | 9.01 |
| 22 | VAL | C | 1.30 | 31.25 | 6.35 | 8.96 |
| 22 | VAL | O | 1.02 | 30.25 | 5.70 | 8.97 |
| 23 | ARG | N | 2.44 | 31.92 | 6.17 | 8.93 |
| 23 | ARG | CA | 3.42 | 31.50 | 5.18 | 8.94 |
| 23 | ARG | CB | 4.62 | 32.45 | 5.23 | 8.90 |
| 23 | ARG | CG | 5.67 | 32.23 | 4.16 | 8.91 |
| 23 | ARG | CD | 6.96 | 32.89 | 4.60 | 8.94 |
| 23 | ARG | NE | 7.46 | 32.22 | 5.80 | 8.98 |
| 23 | ARG | CZ | 7.99 | 32.83 | 6.86 | 8.98 |
| 23 | ARG | NH1 | 8.12 | 34.16 | 6.88 | 8.99 |
| 23 | ARG | NH2 | 8.38 | 32.11 | 7.90 | 8.95 |
| 23 | ARG | C | 3.88 | 30.07 | 5.45 | 9.00 |
| 23 | ARG | O | 3.91 | 29.23 | 4.54 | 9.00 |
| 24 | ASN | N | 4.23 | 29.78 | 6.70 | 9.15 |
| 24 | ASN | CA | 4.69 | 28.44 | 7.05 | 9.21 |
| 24 | ASN | CB | 5.13 | 28.37 | 8.52 | 9.46 |
| 24 | ASN | CG | 6.39 | 29.16 | 8.79 | 9.66 |
| 24 | ASN | OD1 | 7.17 | 29.45 | 7.89 | 9.82 |
| 24 | ASN | ND2 | 6.60 | 29.50 | 10.06 | 9.94 |
| 24 | ASN | C | 3.62 | 27.38 | 6.82 | 9.27 |
| 24 | ASN | O | 3.92 | 26.30 | 6.31 | 9.24 |
| 25 | THR | N | 2.39 | 27.68 | 7.22 | 9.27 |
| 25 | THR | CA | 1.29 | 26.74 | 7.08 | 9.30 |
| 25 | THR | CB | 0.01 | 27.28 | 7.76 | 9.36 |
| 25 | THR | OG1 | 0.31 | 27.61 | 9.13 | 9.55 |
| 25 | THR | CG2 | −1.09 | 26.22 | 7.76 | 9.41 |
| 25 | THR | C | 1.03 | 26.44 | 5.60 | 9.35 |
| 25 | THR | O | 0.87 | 25.28 | 5.21 | 9.34 |
| 26 | MET | N | 1.01 | 27.48 | 4.78 | 9.38 |
| 26 | MET | CA | 0.79 | 27.30 | 3.35 | 9.43 |
| 26 | MET | CB | 0.66 | 28.66 | 2.66 | 9.32 |
| 26 | MET | CG | −0.65 | 29.37 | 2.96 | 9.07 |
| 26 | MET | SD | −0.73 | 31.04 | 2.29 | 8.85 |
| 26 | MET | CE | −1.03 | 30.69 | 0.55 | 8.82 |
| 26 | MET | C | 1.93 | 26.49 | 2.72 | 9.62 |
| 26 | MET | O | 1.68 | 25.58 | 1.92 | 9.69 |
| 27 | GLN | N | 3.17 | 26.81 | 3.06 | 9.86 |
| 27 | GLN | CA | 4.29 | 26.06 | 2.50 | 10.12 |
| 27 | GLN | CB | 5.62 | 26.63 | 3.00 | 10.25 |
| 27 | GLN | CG | 6.83 | 25.82 | 2.53 | 10.36 |
| 27 | GLN | CD | 8.15 | 26.48 | 2.87 | 10.53 |
| 27 | GLN | OE1 | 8.20 | 27.44 | 3.63 | 10.60 |
| 27 | GLN | NE2 | 9.23 | 25.96 | 2.30 | 10.56 |
| 27 | GLN | C | 4.20 | 24.58 | 2.87 | 10.33 |
| 27 | GLN | O | 4.41 | 23.70 | 2.03 | 10.33 |
| 28 | ALA | N | 3.89 | 24.30 | 4.13 | 10.50 |
| 28 | ALA | CA | 3.79 | 22.93 | 4.61 | 10.78 |
| 28 | ALA | CB | 3.52 | 22.91 | 6.10 | 10.75 |
| 28 | ALA | C | 2.70 | 22.15 | 3.88 | 10.99 |
| 28 | ALA | O | 2.80 | 20.93 | 3.73 | 11.11 |
| 29 | ALA | N | 1.66 | 22.86 | 3.44 | 11.17 |
| 29 | ALA | CA | 0.54 | 22.24 | 2.75 | 11.29 |
| 29 | ALA | CB | −0.74 | 23.02 | 3.05 | 11.35 |
| 29 | ALA | C | 0.72 | 22.12 | 1.24 | 11.43 |
| 29 | ALA | O | −0.10 | 21.52 | 0.55 | 11.44 |
| 30 | THR | N | 1.80 | 22.70 | 0.73 | 11.52 |
| 30 | THR | CA | 2.06 | 22.65 | −0.70 | 11.67 |
| 30 | THR | CB | 2.98 | 23.80 | −1.14 | 11.62 |
| 30 | THR | OG1 | 2.42 | 25.06 | −0.74 | 11.60 |
| 30 | THR | CG2 | 3.12 | 23.81 | −2.65 | 11.57 |
| 30 | THR | C | 2.65 | 21.31 | −1.11 | 11.87 |
| 30 | THR | O | 3.82 | 21.02 | −0.86 | 11.99 |
| 31 | LYS | N | 1.82 | 20.48 | −1.74 | 11.99 |
| 31 | LYS | CA | 2.21 | 19.15 | −2.18 | 12.15 |
| 31 | LYS | CB | 1.71 | 18.12 | −1.17 | 12.43 |
| 31 | LYS | CG | 0.21 | 18.19 | −0.96 | 12.97 |
| 31 | LYS | CD | −0.33 | 17.17 | 0.04 | 13.38 |
| 31 | LYS | CE | −0.41 | 15.78 | −0.55 | 13.70 |
| 31 | LYS | NZ | −1.09 | 14.84 | 0.38 | 13.97 |
| 31 | LYS | C | 1.54 | 18.89 | −3.52 | 12.07 |
| 31 | LYS | O | 0.58 | 19.57 | −3.88 | 11.94 |
| 32 | PRO | N | 2.05 | 17.91 | −4.28 | 12.10 |
| 32 | PRO | CD | 3.26 | 17.10 | −4.08 | 12.06 |
| 32 | PRO | CA | 1.42 | 17.61 | −5.58 | 12.11 |
| 32 | PRO | CB | 2.22 | 16.40 | −6.07 | 12.12 |
| 32 | PRO | CG | 3.58 | 16.63 | −5.48 | 12.06 |
| 32 | PRO | C | −0.05 | 17.27 | −5.34 | 12.22 |
| 32 | PRO | O | −0.38 | 16.55 | −4.40 | 12.32 |
| 33 | GLY | N | −0.94 | 17.79 | −6.18 | 12.33 |
| 33 | GLY | CA | −2.34 | 17.49 | −6.01 | 12.37 |
| 33 | GLY | C | −3.20 | 18.57 | −5.40 | 12.38 |
| 33 | GLY | O | −4.43 | 18.52 | −5.51 | 12.55 |
| 34 | ILE | N | −2.59 | 19.55 | −4.74 | 12.25 |
| 34 | ILE | CA | −3.37 | 20.63 | −4.15 | 12.14 |
| 34 | ILE | CB | −2.77 | 21.09 | −2.79 | 12.07 |
| 34 | ILE | CG2 | −1.49 | 21.86 | −3.02 | 12.01 |
| 34 | ILE | CG1 | −3.77 | 21.97 | −2.05 | 12.05 |
| 34 | ILE | CD1 | −3.37 | 22.28 | −0.62 | 12.08 |
| 34 | ILE | C | −3.37 | 21.79 | −5.14 | 12.00 |
| 34 | ILE | O | −2.40 | 21.98 | −5.88 | 12.04 |
| 35 | THR | N | −4.46 | 22.56 | −5.19 | 11.92 |
| 35 | THR | CA | −4.53 | 23.70 | −6.10 | 11.90 |
| 35 | THR | CB | −5.96 | 23.95 | −6.59 | 11.88 |
| 35 | THR | OG1 | −6.80 | 24.28 | −5.47 | 11.85 |
| 35 | THR | CG2 | −6.51 | 22.73 | −7.31 | 11.94 |
| 35 | THR | C | −4.07 | 24.94 | −5.35 | 11.78 |
| 35 | THR | O | −4.11 | 24.98 | −4.12 | 11.74 |
| 36 | THR | N | −3.66 | 25.96 | −6.09 | 11.80 |
| 36 | THR | CA | −3.22 | 27.18 | −5.43 | 11.82 |
| 36 | THR | CB | −2.53 | 28.15 | −6.42 | 11.77 |
| 36 | THR | OG1 | −3.41 | 28.45 | −7.51 | 11.73 |
| 36 | THR | CG2 | −1.27 | 27.51 | −6.97 | 11.66 |
| 36 | THR | C | −4.39 | 27.86 | −4.71 | 11.88 |
| 36 | THR | O | −4.19 | 28.52 | −3.70 | 11.80 |
| 37 | LYS | N | −5.60 | 27.66 | −5.21 | 11.99 |
| 37 | LYS | CA | −6.78 | 28.24 | −4.56 | 12.18 |
| 37 | LYS | CB | −8.04 | 27.96 | −5.38 | 12.39 |
| 37 | LYS | CG | −9.34 | 28.44 | −4.73 | 12.76 |
| 37 | LYS | CD | −9.36 | 29.95 | −4.57 | 13.07 |
| 37 | LYS | CE | −10.66 | 30.44 | −3.94 | 13.28 |
| 37 | LYS | NZ | −10.66 | 31.91 | −3.75 | 13.53 |
| 37 | LYS | C | −6.93 | 27.62 | −3.17 | 12.12 |
| 37 | LYS | O | −7.22 | 28.31 | −2.19 | 12.14 |
| 38 | GLU | N | −6.75 | 26.30 | −3.08 | 12.09 |
| 38 | GLU | CA | −6.85 | 25.61 | −1.81 | 12.08 |
| 38 | GLU | CB | −6.67 | 24.11 | −2.01 | 12.41 |
| 38 | GLU | CG | −7.87 | 23.43 | −2.62 | 12.97 |
| 38 | GLU | CD | −7.56 | 22.00 | −2.95 | 13.28 |
| 38 | GLU | OE1 | −6.94 | 21.76 | −4.01 | 13.62 |
| 38 | GLU | OE2 | −7.92 | 21.11 | −2.14 | 13.67 |
| 38 | GLU | C | −5.82 | 26.12 | −0.81 | 11.91 |
| 38 | GLU | O | −6.10 | 26.21 | 0.38 | 11.83 |
| 39 | LEU | N | −4.63 | 26.45 | −1.31 | 11.71 |
| 39 | LEU | CA | −3.59 | 26.98 | −0.45 | 11.58 |
| 39 | LEU | CB | −2.27 | 27.06 | −1.22 | 11.48 |
| 39 | LEU | CG | −1.60 | 25.72 | −1.55 | 11.42 |
| 39 | LEU | CD1 | −0.39 | 25.97 | −2.44 | 11.41 |
| 39 | LEU | CD2 | −1.18 | 25.02 | −0.27 | 11.41 |
| 39 | LEU | C | −4.00 | 28.36 | 0.03 | 11.54 |
| 39 | LEU | O | −3.82 | 28.70 | 1.20 | 11.42 |
| 40 | ASP | N | −4.58 | 29.15 | −0.87 | 11.58 |
| 40 | ASP | CA | −5.03 | 30.50 | −0.55 | 11.69 |
| 40 | ASP | CB | −5.56 | 31.18 | −1.81 | 11.71 |
| 40 | ASP | CG | −5.75 | 32.68 | −1.64 | 11.73 |
| 40 | ASP | OD1 | −4.75 | 33.37 | −1.35 | 11.76 |
| 40 | ASP | OD2 | −6.89 | 33.17 | −1.79 | 11.87 |
| 40 | ASP | C | −6.12 | 30.45 | 0.52 | 11.76 |
| 40 | ASP | O | −6.22 | 31.36 | 1.34 | 11.76 |
| 41 | ASN | N | −6.92 | 29.39 | 0.51 | 11.94 |
| 41 | ASN | CA | −8.00 | 29.26 | 1.48 | 12.11 |
| 41 | ASN | CB | −8.88 | 28.04 | 1.16 | 12.08 |
| 41 | ASN | CG | −9.75 | 28.26 | −0.06 | 12.15 |
| 41 | ASN | OD1 | −9.97 | 29.38 | −0.49 | 12.25 |
| 41 | ASN | ND2 | −10.27 | 27.17 | −0.61 | 12.08 |
| 41 | ASN | C | −7.44 | 29.12 | 2.89 | 12.21 |
| 41 | ASN | O | −8.10 | 29.48 | 3.87 | 12.29 |
| 42 | ILE | N | −6.23 | 28.59 | 2.99 | 12.31 |
| 42 | ILE | CA | −5.59 | 28.44 | 4.29 | 12.49 |
| 42 | ILE | CB | −4.25 | 27.68 | 4.16 | 12.45 |
| 42 | ILE | CG2 | −3.48 | 27.75 | 5.47 | 12.47 |
| 42 | ILE | CG1 | −4.52 | 26.23 | 3.77 | 12.45 |
| 42 | ILE | CD1 | −3.26 | 25.40 | 3.55 | 12.39 |
| 42 | ILE | C | −5.35 | 29.83 | 4.85 | 12.64 |
| 42 | ILE | O | −5.60 | 30.08 | 6.03 | 12.59 |
| 43 | ALA | N | −4.89 | 30.75 | 4.01 | 12.86 |
| 43 | ALA | CA | −4.66 | 32.12 | 4.46 | 13.08 |
| 43 | ALA | CB | −3.99 | 32.94 | 3.36 | 13.03 |
| 43 | ALA | C | −5.99 | 32.76 | 4.86 | 13.31 |
| 43 | ALA | O | −6.06 | 33.47 | 5.86 | 13.27 |
| 44 | LYS | N | −7.04 | 32.51 | 4.08 | 13.59 |
| 44 | LYS | CA | −8.34 | 33.08 | 4.40 | 13.91 |
| 44 | LYS | CB | −9.41 | 32.57 | 3.42 | 14.05 |
| 44 | LYS | CG | −10.82 | 33.11 | 3.71 | 14.28 |
| 44 | LYS | CD | −11.85 | 32.54 | 2.73 | 14.54 |
| 44 | LYS | CE | −13.27 | 32.99 | 3.06 | 14.74 |
| 44 | LYS | NZ | −13.48 | 34.45 | 2.84 | 15.04 |
| 44 | LYS | C | −8.73 | 32.69 | 5.82 | 14.13 |
| 44 | LYS | O | −9.10 | 33.54 | 6.63 | 14.12 |
| 45 | GLU | N | −8.65 | 31.40 | 6.13 | 14.37 |
| 45 | GLU | CA | −9.02 | 30.91 | 7.45 | 14.69 |
| 45 | GLU | CB | −8.98 | 29.39 | 7.48 | 15.07 |
| 45 | GLU | CG | −10.02 | 28.74 | 6.57 | 15.74 |
| 45 | GLU | CD | −11.42 | 29.26 | 6.82 | 16.05 |
| 45 | GLU | OE1 | −11.90 | 29.15 | 7.97 | 16.40 |
| 45 | GLU | OE2 | −12.05 | 29.77 | 5.87 | 16.39 |
| 45 | GLU | C | −8.14 | 31.47 | 8.57 | 14.69 |
| 45 | GLU | O | −8.65 | 31.93 | 9.59 | 14.68 |
| 46 | LEU | N | −6.82 | 31.43 | 8.38 | 14.73 |
| 46 | LEU | CA | −5.92 | 31.94 | 9.41 | 14.79 |
| 46 | LEU | CB | −4.47 | 31.57 | 9.09 | 14.74 |
| 46 | LEU | CG | −4.14 | 30.07 | 9.20 | 14.80 |
| 46 | LEU | CD1 | −2.67 | 29.85 | 8.87 | 14.78 |
| 46 | LEU | CD2 | −4.45 | 29.57 | 10.61 | 14.81 |
| 46 | LEU | C | −6.04 | 33.44 | 9.62 | 14.78 |
| 46 | LEU | O | −6.02 | 33.91 | 10.76 | 14.79 |
| 47 | PHE | N | −6.18 | 34.21 | 8.54 | 14.85 |
| 47 | PHE | CA | −6.32 | 35.66 | 8.70 | 14.98 |
| 47 | PHE | CB | −6.50 | 36.37 | 7.35 | 14.71 |
| 47 | PHE | CG | −5.22 | 36.53 | 6.56 | 14.45 |
| 47 | PHE | CD1 | −3.97 | 36.55 | 7.19 | 14.33 |
| 47 | PHE | CD2 | −5.27 | 36.72 | 5.18 | 14.30 |
| 47 | PHE | CE1 | −2.80 | 36.76 | 6.45 | 14.26 |
| 47 | PHE | CE2 | −4.11 | 36.92 | 4.43 | 14.28 |
| 47 | PHE | CZ | −2.87 | 36.95 | 5.07 | 14.21 |
| 47 | PHE | C | −7.54 | 35.93 | 9.59 | 15.29 |
| 47 | PHE | O | −7.49 | 36.75 | 10.50 | 15.21 |
| 48 | GLU | N | −8.64 | 35.24 | 9.30 | 15.64 |
| 48 | GLU | CA | −9.87 | 35.39 | 10.08 | 16.08 |
| 48 | GLU | CB | −10.96 | 34.48 | 9.51 | 16.42 |
| 48 | GLU | CG | −12.23 | 34.42 | 10.33 | 17.03 |
| 48 | GLU | CD | −12.84 | 35.78 | 10.56 | 17.34 |
| 48 | GLU | OE1 | −12.79 | 36.62 | 9.63 | 17.70 |
| 48 | GLU | OE2 | −13.37 | 36.02 | 11.67 | 17.62 |
| 48 | GLU | C | −9.62 | 35.02 | 11.53 | 16.18 |
| 48 | GLU | O | −10.05 | 35.72 | 12.45 | 16.19 |
| 49 | GLU | N | −8.91 | 33.92 | 11.73 | 16.29 |
| 49 | GLU | CA | −8.59 | 33.41 | 13.06 | 16.47 |
| 49 | GLU | CB | −7.83 | 32.09 | 12.94 | 16.79 |
| 49 | GLU | CG | −7.60 | 31.39 | 14.26 | 17.31 |
| 49 | GLU | CD | −6.79 | 30.11 | 14.10 | 17.56 |
| 49 | GLU | OE1 | −7.02 | 29.40 | 13.09 | 17.88 |
| 49 | GLU | OE2 | −5.95 | 29.82 | 14.97 | 17.88 |
| 49 | GLU | C | −7.77 | 34.38 | 13.91 | 16.42 |
| 49 | GLU | O | −8.02 | 34.54 | 15.10 | 16.42 |
| 50 | TYR | N | −6.79 | 35.03 | 13.28 | 16.32 |
| 50 | TYR | CA | −5.90 | 35.96 | 13.98 | 16.20 |
| 50 | TYR | CB | −4.47 | 35.82 | 13.45 | 16.37 |
| 50 | TYR | CG | −3.83 | 34.48 | 13.77 | 16.61 |
| 50 | TYR | CD1 | −3.47 | 34.15 | 15.08 | 16.73 |
| 50 | TYR | CE1 | −2.89 | 32.92 | 15.37 | 16.88 |
| 50 | TYR | CD2 | −3.60 | 33.55 | 12.77 | 16.74 |
| 50 | TYR | CE2 | −3.02 | 32.31 | 13.05 | 16.84 |
| 50 | TYR | CZ | −2.67 | 32.00 | 14.36 | 16.89 |
| 50 | TYR | OH | −2.09 | 30.78 | 14.64 | 17.06 |
| 50 | TYR | C | −6.33 | 37.42 | 13.91 | 16.00 |
| 50 | TYR | O | −5.60 | 38.30 | 14.37 | 15.99 |
| 51 | GLY | N | −7.49 | 37.68 | 13.33 | 15.80 |
| 51 | GLY | CA | −7.99 | 39.04 | 13.23 | 15.52 |
| 51 | GLY | C | −7.25 | 39.95 | 12.28 | 15.36 |
| 51 | GLY | O | −7.20 | 41.16 | 12.49 | 15.33 |
| 52 | ALA | N | −6.67 | 39.38 | 11.23 | 15.16 |
| 52 | ALA | CA | −5.94 | 40.17 | 10.25 | 15.02 |
| 52 | ALA | CB | −4.55 | 39.57 | 10.01 | 14.95 |
| 52 | ALA | C | −6.70 | 40.23 | 8.93 | 14.92 |
| 52 | ALA | O | −7.66 | 39.49 | 8.72 | 14.84 |
| 53 | ILE | N | −6.25 | 41.12 | 8.04 | 14.89 |
| 53 | ILE | CA | −6.87 | 41.29 | 6.73 | 14.92 |
| 53 | ILE | CB | −7.61 | 42.66 | 6.64 | 15.05 |
| 53 | ILE | CG2 | −8.02 | 42.93 | 5.20 | 15.17 |
| 53 | ILE | CG1 | −8.82 | 42.65 | 7.56 | 15.21 |
| 53 | ILE | CD1 | −9.85 | 41.59 | 7.21 | 15.39 |
| 53 | ILE | C | −5.80 | 41.24 | 5.64 | 14.88 |
| 53 | ILE | O | −4.70 | 41.77 | 5.81 | 14.72 |
| 54 | SER | N | −6.13 | 40.58 | 4.54 | 14.87 |
| 54 | SER | CA | −5.23 | 40.45 | 3.40 | 15.01 |
| 54 | SER | CB | −5.90 | 39.62 | 2.30 | 14.89 |
| 54 | SER | OG | −5.27 | 39.86 | 1.05 | 14.78 |
| 54 | SER | C | −4.86 | 41.81 | 2.83 | 15.15 |
| 54 | SER | O | −5.74 | 42.61 | 2.48 | 15.16 |
| 55 | ALA | N | −3.56 | 42.08 | 2.72 | 15.34 |
| 55 | ALA | CA | −3.09 | 43.34 | 2.17 | 15.57 |
| 55 | ALA | CB | −1.59 | 43.49 | 2.40 | 15.51 |
| 55 | ALA | C | −3.41 | 43.40 | 0.68 | 15.80 |
| 55 | ALA | O | −3.78 | 44.45 | 0.16 | 15.79 |
| 56 | PRO | N | −3.26 | 42.27 | −0.03 | 16.00 |
| 56 | PRO | CD | −2.50 | 41.06 | 0.30 | 16.01 |
| 56 | PRO | CA | −3.58 | 42.30 | −1.46 | 16.26 |
| 56 | PRO | CB | −3.23 | 40.89 | −1.91 | 16.17 |
| 56 | PRO | CG | −2.04 | 40.57 | −1.06 | 16.07 |
| 56 | PRO | C | −5.02 | 42.68 | −1.76 | 16.53 |
| 56 | PRO | O | −5.30 | 43.45 | −2.68 | 16.59 |
| 57 | ILE | N | −5.94 | 42.14 | −0.98 | 16.85 |
| 57 | ILE | CA | −7.35 | 42.44 | −1.18 | 17.17 |
| 57 | ILE | CB | −8.25 | 41.46 | −0.40 | 17.13 |
| 57 | ILE | CG2 | −9.70 | 41.91 | −0.47 | 17.13 |
| 57 | ILE | CG1 | −8.08 | 40.04 | −0.95 | 17.10 |
| 57 | ILE | CD1 | −8.77 | 38.97 | −0.13 | 17.01 |
| 57 | ILE | C | −7.68 | 43.85 | −0.73 | 17.50 |
| 57 | ILE | O | −8.34 | 44.61 | −1.44 | 17.57 |
| 58 | HIS | N | −7.19 | 44.21 | 0.46 | 17.84 |
| 58 | HIS | CA | −7.44 | 45.52 | 1.04 | 18.18 |
| 58 | HIS | CB | −6.97 | 45.52 | 2.50 | 18.23 |
| 58 | HIS | CG | −7.19 | 46.82 | 3.22 | 18.32 |
| 58 | HIS | CD2 | −8.15 | 47.20 | 4.09 | 18.38 |
| 58 | HIS | ND1 | −6.34 | 47.89 | 3.09 | 18.37 |
| 58 | HIS | CE1 | −6.77 | 48.88 | 3.85 | 18.38 |
| 58 | HIS | NE2 | −7.87 | 48.49 | 4.47 | 18.39 |
| 58 | HIS | C | −6.82 | 46.70 | 0.29 | 18.38 |
| 58 | HIS | O | −7.51 | 47.67 | 0.00 | 18.43 |
| 59 | ASP | N | −5.53 | 46.60 | −0.01 | 18.61 |
| 59 | ASP | CA | −4.80 | 47.67 | −0.70 | 18.84 |
| 59 | ASP | CB | −3.30 | 47.59 | −0.37 | 18.99 |
| 59 | ASP | CG | −2.96 | 48.10 | 1.03 | 19.14 |
| 59 | ASP | OD1 | −3.67 | 47.75 | 1.99 | 19.19 |
| 59 | ASP | OD2 | −1.97 | 48.86 | 1.18 | 19.27 |
| 59 | ASP | C | −4.96 | 47.69 | −2.23 | 18.90 |
| 59 | ASP | O | −4.99 | 48.77 | −2.83 | 19.03 |
| 60 | GLU | N | −5.06 | 46.52 | −2.86 | 18.91 |
| 60 | GLU | CA | −5.16 | 46.45 | −4.32 | 18.85 |
| 60 | GLU | CB | −3.95 | 45.70 | −4.88 | 19.15 |
| 60 | GLU | CG | −2.62 | 46.06 | −4.26 | 19.60 |
| 60 | GLU | CD | −2.31 | 47.53 | −4.34 | 19.85 |
| 60 | GLU | OE1 | −2.50 | 48.12 | −5.44 | 20.14 |
| 60 | GLU | OE2 | −1.88 | 48.11 | −3.32 | 20.10 |
| 60 | GLU | C | −6.41 | 45.81 | −4.91 | 18.59 |
| 60 | GLU | O | −6.59 | 45.82 | −6.13 | 18.61 |
| 61 | ASN | N | −7.28 | 45.27 | −4.07 | 18.31 |
| 61 | ASN | CA | −8.48 | 44.59 | −4.54 | 17.99 |
| 61 | ASN | CB | −9.39 | 45.55 | −5.33 | 18.12 |
| 61 | ASN | CG | −10.80 | 45.00 | −5.51 | 18.17 |
| 61 | ASN | OD1 | −11.22 | 44.11 | −4.78 | 18.25 |
| 61 | ASN | ND2 | −11.53 | 45.55 | −6.48 | 18.20 |
| 61 | ASN | C | −8.02 | 43.43 | −5.42 | 17.72 |
| 61 | ASN | O | −8.64 | 43.10 | −6.43 | 17.76 |
| 62 | PHE | N | −6.91 | 42.82 | −5.01 | 17.30 |
| 62 | PHE | CA | −6.31 | 41.68 | −5.69 | 16.83 |
| 62 | PHE | CB | −4.96 | 41.34 | −5.05 | 16.89 |
| 62 | PHE | CG | −4.23 | 40.18 | −5.70 | 16.90 |
| 62 | PHE | CD1 | −3.63 | 40.33 | −6.94 | 16.96 |
| 62 | PHE | CD2 | −4.14 | 38.95 | −5.05 | 16.94 |
| 62 | PHE | CE1 | −2.94 | 39.27 | −7.53 | 16.96 |
| 62 | PHE | CE2 | −3.45 | 37.89 | −5.63 | 16.96 |
| 62 | PHE | CZ | −2.85 | 38.05 | −6.87 | 16.97 |
| 62 | PHE | C | −7.28 | 40.52 | −5.52 | 16.47 |
| 62 | PHE | O | −7.83 | 40.32 | −4.43 | 16.39 |
| 63 | PRO | N | −7.50 | 39.73 | −6.58 | 16.11 |
| 63 | PRO | CD | −7.04 | 39.90 | −7.97 | 16.07 |
| 63 | PRO | CA | −8.42 | 38.60 | −6.47 | 15.77 |
| 63 | PRO | CB | −8.77 | 38.31 | −7.92 | 15.90 |
| 63 | PRO | CG | −7.49 | 38.60 | −8.62 | 15.98 |
| 63 | PRO | C | −7.79 | 37.39 | −5.77 | 15.37 |
| 63 | PRO | O | −7.65 | 36.32 | −6.37 | 15.39 |
| 64 | GLY | N | −7.41 | 37.58 | −4.51 | 14.88 |
| 64 | GLY | CA | −6.80 | 36.50 | −3.75 | 14.26 |
| 64 | GLY | C | −6.27 | 36.97 | −2.41 | 13.78 |
| 64 | GLY | O | −5.83 | 38.12 | −2.28 | 13.74 |
| 65 | GLN | N | −6.30 | 36.10 | −1.40 | 13.36 |
| 65 | GLN | CA | −5.80 | 36.46 | −0.07 | 12.86 |
| 65 | GLN | CB | −6.04 | 35.31 | 0.91 | 12.86 |
| 65 | GLN | CG | −7.50 | 34.96 | 1.17 | 12.99 |
| 65 | GLN | CD | −8.24 | 36.03 | 1.93 | 13.11 |
| 65 | GLN | OE1 | −7.68 | 36.70 | 2.80 | 13.08 |
| 65 | GLN | NE2 | −9.53 | 36.18 | 1.63 | 13.16 |
| 65 | GLN | C | −4.30 | 36.73 | −0.15 | 12.53 |
| 65 | GLN | O | −3.80 | 37.69 | 0.45 | 12.41 |
| 66 | THR | N | −3.61 | 35.88 | −0.90 | 12.14 |
| 66 | THR | CA | −2.16 | 35.96 | −1.06 | 11.77 |
| 66 | THR | CB | −1.48 | 34.80 | −0.32 | 11.76 |
| 66 | THR | OG1 | −1.84 | 33.56 | −0.95 | 11.65 |
| 66 | THR | CG2 | −1.93 | 34.77 | 1.13 | 11.69 |
| 66 | THR | C | −1.76 | 35.83 | −2.53 | 11.62 |
| 66 | THR | O | −2.61 | 35.60 | −3.39 | 11.52 |
| 67 | CYS | N | −0.47 | 35.96 | −2.80 | 11.43 |
| 67 | CYS | CA | 0.06 | 35.80 | −4.15 | 11.27 |
| 67 | CYS | CB | 1.01 | 36.94 | −4.52 | 11.39 |
| 67 | CYS | SG | 0.24 | 38.57 | −4.63 | 11.80 |
| 67 | CYS | C | 0.81 | 34.49 | −4.19 | 11.08 |
| 67 | CYS | O | 1.70 | 34.25 | −3.38 | 10.93 |
| 68 | ILE | N | 0.44 | 33.62 | −5.12 | 10.86 |
| 68 | ILE | CA | 1.10 | 32.33 | −5.26 | 10.79 |
| 68 | ILE | CB | 0.15 | 31.18 | −4.91 | 10.71 |
| 68 | ILE | CG2 | 0.87 | 29.84 | −5.09 | 10.62 |
| 68 | ILE | CG1 | −0.33 | 31.31 | −3.46 | 10.66 |
| 68 | ILE | CD1 | −1.37 | 30.30 | −3.05 | 10.71 |
| 68 | ILE | C | 1.53 | 32.26 | −6.71 | 10.80 |
| 68 | ILE | O | 0.70 | 32.23 | −7.62 | 10.86 |
| 69 | SER | N | 2.84 | 32.23 | −6.92 | 10.83 |
| 69 | SER | CA | 3.41 | 32.22 | −8.27 | 10.87 |
| 69 | SER | CB | 4.36 | 33.42 | −8.41 | 10.79 |
| 69 | SER | CG | 3.73 | 34.61 | −7.95 | 10.68 |
| 69 | SER | C | 4.16 | 30.93 | −8.53 | 10.96 |
| 69 | SER | O | 5.03 | 30.54 | −7.75 | 10.94 |
| 70 | VAL | N | 3.83 | 30.26 | −9.63 | 11.06 |
| 70 | VAL | CA | 4.46 | 28.98 | −9.94 | 11.23 |
| 70 | VAL | CB | 3.41 | 27.84 | −9.95 | 11.16 |
| 70 | VAL | CG1 | 4.08 | 26.50 | −10.16 | 11.21 |
| 70 | VAL | CG2 | 2.64 | 27.84 | −8.64 | 11.25 |
| 70 | VAL | C | 5.23 | 28.93 | −11.26 | 11.38 |
| 70 | VAL | O | 4.77 | 29.43 | −12.28 | 11.43 |
| 71 | ASN | N | 6.42 | 28.32 | −11.19 | 11.49 |
| 71 | ASN | CA | 7.31 | 28.12 | −12.33 | 11.78 |
| 71 | ASN | CB | 6.79 | 26.96 | −13.19 | 11.77 |
| 71 | ASN | CG | 6.79 | 25.64 | −12.43 | 11.74 |
| 71 | ASN | OD1 | 7.67 | 25.39 | −11.60 | 11.67 |
| 71 | ASN | ND2 | 5.82 | 24.78 | −12.72 | 11.76 |
| 71 | ASN | C | 7.59 | 29.33 | −13.21 | 11.95 |
| 71 | ASN | O | 8.51 | 30.10 | −12.94 | 12.06 |
| 72 | GLU | N | 6.81 | 29.51 | −14.28 | 12.13 |
| 72 | GLU | CA | 7.04 | 30.64 | −15.16 | 12.27 |
| 72 | GLU | CB | 6.23 | 30.48 | −16.46 | 12.36 |
| 72 | GLU | CG | 4.74 | 30.78 | −16.35 | 12.60 |
| 72 | GLU | CD | 3.93 | 29.63 | −15.77 | 12.64 |
| 72 | GLU | OE1 | 4.50 | 28.55 | −15.49 | 12.77 |
| 72 | GLU | OE2 | 2.71 | 29.82 | −15.58 | 12.82 |
| 72 | GLU | C | 6.69 | 31.96 | −14.48 | 12.29 |
| 72 | GLU | O | 7.20 | 33.02 | −14.86 | 12.30 |
| 73 | GLU | N | 5.82 | 31.90 | −13.47 | 12.35 |
| 73 | GLU | CA | 5.42 | 33.09 | −12.74 | 12.44 |
| 73 | GLU | CB | 4.03 | 32.90 | −12.13 | 12.54 |
| 73 | GLU | CG | 2.96 | 32.56 | −13.14 | 12.67 |
| 73 | GLU | CD | 1.66 | 32.20 | −12.48 | 12.81 |
| 73 | GLU | OE1 | 1.71 | 31.57 | −11.40 | 12.80 |
| 73 | GLU | OE2 | 0.59 | 32.52 | −13.05 | 12.97 |
| 73 | GLU | C | 6.41 | 33.40 | −11.64 | 12.46 |
| 73 | GLU | O | 6.76 | 32.52 | −10.84 | 12.46 |
| 74 | VAL | N | 6.87 | 34.64 | −11.60 | 12.51 |
| 74 | VAL | CA | 7.84 | 35.09 | −10.61 | 12.66 |
| 74 | VAL | CB | 8.83 | 36.10 | −11.24 | 12.58 |
| 74 | VAL | CG1 | 9.82 | 36.60 | −10.21 | 12.64 |
| 74 | VAL | CG2 | 9.55 | 35.46 | −12.42 | 12.65 |
| 74 | VAL | C | 7.18 | 35.75 | −9.40 | 12.77 |
| 74 | VAL | O | 7.62 | 35.58 | −8.26 | 12.66 |
| 75 | ALA | N | 6.12 | 36.51 | −9.66 | 12.86 |
| 75 | ALA | CA | 5.43 | 37.23 | −8.59 | 13.04 |
| 75 | ALA | CB | 6.23 | 38.48 | −8.23 | 13.11 |
| 75 | ALA | C | 4.02 | 37.62 | −8.98 | 13.24 |
| 75 | ALA | O | 3.67 | 37.64 | −10.16 | 13.20 |
| 76 | HIS | N | 3.21 | 37.92 | −7.97 | 13.43 |
| 76 | HIS | CA | 1.83 | 38.35 | −8.15 | 13.63 |
| 76 | HIS | CB | 1.80 | 39.66 | −8.94 | 14.05 |
| 76 | HIS | CG | 2.43 | 40.81 | −8.23 | 14.51 |
| 76 | HIS | CD2 | 2.20 | 42.15 | −8.31 | 14.67 |
| 76 | HIS | ND1 | 3.47 | 40.66 | −7.34 | 14.73 |
| 76 | HIS | CE1 | 3.86 | 41.85 | −6.91 | 14.81 |
| 76 | HIS | NE2 | 3.10 | 42.77 | −7.48 | 14.89 |
| 76 | HIS | C | 0.92 | 37.32 | −8.82 | 13.65 |
| 76 | HIS | O | −0.07 | 37.68 | −9.45 | 13.62 |
| 77 | GLY | N | 1.24 | 36.03 | −8.68 | 13.56 |
| 77 | GLY | CA | 0.40 | 35.01 | −9.27 | 13.53 |
| 77 | GLY | C | −0.95 | 34.94 | −8.57 | 13.57 |
| 77 | GLY | O | −1.03 | 35.10 | −7.36 | 13.44 |
| 78 | ILE | N | −2.01 | 34.70 | −9.34 | 13.65 |
| 78 | ILE | CA | −3.36 | 34.63 | −8.79 | 13.67 |
| 78 | ILE | CB | −4.41 | 35.12 | −9.81 | 13.74 |
| 78 | ILE | CG2 | −5.77 | 35.22 | −9.15 | 13.79 |
| 78 | ILE | CG1 | −4.01 | 36.47 | −10.40 | 13.79 |
| 78 | ILE | CD1 | −4.88 | 36.91 | −11.58 | 13.86 |
| 78 | ILE | O | −3.75 | 33.21 | −8.38 | 13.80 |
| 78 | ILE | O | −3.73 | 32.31 | −9.21 | 13.76 |
| 79 | PRO | N | −4.10 | 32.99 | −7.10 | 13.88 |
| 79 | PRO | CD | −4.06 | 33.92 | −5.96 | 13.83 |
| 79 | PRO | CA | −4.49 | 31.65 | −6.68 | 13.82 |
| 79 | PRO | CB | −4.93 | 31.86 | −5.23 | 13.87 |
| 79 | PRO | CG | −4.00 | 32.97 | −4.77 | 13.87 |
| 79 | PRO | C | −5.64 | 31.20 | −7.59 | 14.00 |
| 79 | PRO | O | −6.54 | 31.99 | −7.89 | 14.07 |
| 80 | SER | N | −5.60 | 29.95 | −8.04 | 14.07 |
| 80 | SER | CA | −6.61 | 29.45 | −8.96 | 14.16 |
| 80 | SER | CB | −6.23 | 29.87 | −10.38 | 14.16 |
| 80 | SER | OG | −5.05 | 29.19 | −10.78 | 14.14 |
| 80 | SER | C | −6.72 | 27.93 | −8.92 | 14.27 |
| 80 | SER | O | −6.23 | 27.28 | −8.01 | 14.26 |
| 81 | LYS | N | −7.38 | 27.37 | −9.94 | 14.37 |
| 81 | LYS | CA | −7.56 | 25.93 | −10.03 | 14.49 |
| 81 | LYS | CB | −8.68 | 25.61 | −11.03 | 14.81 |
| 81 | LYS | CG | −10.01 | 26.25 | −10.65 | 15.25 |
| 81 | LYS | CD | −11.02 | 26.18 | −11.79 | 15.64 |
| 81 | LYS | CE | −12.31 | 26.92 | −11.44 | 15.87 |
| 81 | LYS | NZ | −13.25 | 26.97 | −12.59 | 16.08 |
| 81 | LYS | C | −6.27 | 25.23 | −10.44 | 14.34 |
| 81 | LYS | O | −6.22 | 24.01 | −10.57 | 14.37 |
| 82 | ARG | N | −5.21 | 26.02 | −10.62 | 14.20 |
| 82 | ARG | CA | −3.92 | 25.46 | −11.01 | 14.03 |
| 82 | ARG | CB | −2.88 | 26.57 | −11.14 | 14.03 |
| 82 | ARG | CG | −1.50 | 26.06 | −11.52 | 14.05 |
| 82 | ARG | CD | −0.53 | 27.18 | −11.80 | 14.04 |
| 82 | ARG | NE | 0.72 | 26.68 | −12.37 | 14.08 |
| 82 | ARG | CZ | 1.54 | 27.41 | −13.11 | 14.04 |
| 82 | ARG | NH1 | 1.26 | 28.67 | −13.37 | 14.07 |
| 82 | ARG | NH2 | 2.65 | 26.87 | −13.61 | 14.04 |
| 82 | ARG | C | −3.47 | 24.44 | −9.97 | 13.93 |
| 82 | ARG | O | −3.39 | 24.75 | −8.78 | 13.83 |
| 83 | VAL | N | −3.16 | 23.23 | −10.42 | 13.85 |
| 83 | VAL | CA | −2.72 | 22.17 | −9.53 | 13.77 |
| 83 | VAL | CB | −3.25 | 20.79 | −9.98 | 13.89 |
| 83 | VAL | CG1 | −2.60 | 20.39 | −11.29 | 14.05 |
| 83 | VAL | CG2 | −2.99 | 19.75 | −8.91 | 13.96 |
| 83 | VAL | C | −1.20 | 22.10 | −9.42 | 13.63 |
| 83 | VAL | O | −0.49 | 22.17 | −10.43 | 13.57 |
| 84 | ILE | N | −0.71 | 21.99 | −8.19 | 13.53 |
| 84 | ILE | CA | 0.72 | 21.88 | −7.92 | 13.43 |
| 84 | ILE | CB | 1.01 | 22.04 | −6.41 | 13.41 |
| 84 | ILE | CG2 | 2.48 | 21.71 | −6.13 | 13.36 |
| 84 | ILE | CG1 | 0.66 | 23.46 | −5.95 | 13.40 |
| 84 | ILE | CD1 | 1.46 | 24.56 | −6.63 | 13.43 |
| 84 | ILE | C | 1.19 | 20.51 | −8.38 | 13.40 |
| 84 | ILE | O | 0.51 | 19.51 | −8.17 | 13.33 |
| 85 | ARG | N | 2.37 | 20.47 | −8.99 | 13.37 |
| 85 | ARG | CA | 2.95 | 19.23 | −9.48 | 13.41 |
| 85 | ARG | CB | 3.03 | 19.25 | −11.02 | 13.72 |
| 85 | ARG | CG | 1.69 | 19.35 | −11.72 | 14.26 |
| 85 | ARG | CD | 0.82 | 18.14 | −11.41 | 14.77 |
| 85 | ARG | NE | 1.38 | 16.91 | −11.94 | 15.32 |
| 85 | ARG | CZ | 1.29 | 16.52 | −13.21 | 15.49 |
| 85 | ARG | NH1 | 0.65 | 17.28 | −14.09 | 15.73 |
| 85 | ARG | NH2 | 1.85 | 15.38 | −13.61 | 15.78 |
| 85 | ARG | C | 4.35 | 19.02 | −8.92 | 13.25 |
| 85 | ARG | O | 5.02 | 19.97 | −8.51 | 13.18 |
| 86 | GLU | N | 4.79 | 17.77 | −8.90 | 13.02 |
| 86 | GLU | CA | 6.12 | 17.41 | −8.42 | 12.82 |
| 86 | GLU | CB | 6.36 | 15.92 | −8.65 | 13.02 |
| 86 | GLU | CG | 7.71 | 15.40 | −8.18 | 13.26 |
| 86 | GLU | CD | 7.82 | 15.28 | −6.68 | 13.38 |
| 86 | GLU | OE1 | 6.81 | 15.50 | −5.97 | 13.63 |
| 86 | GLU | OE2 | 8.93 | 14.95 | −6.19 | 13.48 |
| 86 | GLU | C | 7.13 | 18.23 | −9.21 | 12.57 |
| 86 | GLU | O | 7.04 | 18.32 | −10.44 | 12.61 |
| 87 | GLY | N | 8.09 | 18.83 | −8.51 | 12.27 |
| 87 | GLY | CA | 9.11 | 19.62 | −9.17 | 11.85 |
| 87 | GLY | C | 8.81 | 21.10 | −9.38 | 11.54 |
| 87 | GLY | O | 9.67 | 21.84 | −9.83 | 11.56 |
| 88 | ASP | N | 7.59 | 21.53 | −9.05 | 11.25 |
| 88 | ASP | CA | 7.24 | 22.94 | −9.22 | 10.94 |
| 88 | ASP | CB | 5.76 | 23.18 | −8.88 | 11.06 |
| 88 | ASP | CG | 4.80 | 22.75 | −9.97 | 11.19 |
| 88 | ASP | OD1 | 5.25 | 22.32 | −11.06 | 11.42 |
| 88 | ASP | OD2 | 3.58 | 22.86 | −9.73 | 11.29 |
| 88 | ASP | C | 8.06 | 23.86 | −8.33 | 10.60 |
| 88 | ASP | O | 8.41 | 23.50 | −7.20 | 10.48 |
| 89 | LEU | N | 8.38 | 25.05 | −8.84 | 10.34 |
| 89 | LEU | CA | 9.09 | 26.06 | −8.07 | 10.13 |
| 89 | LEU | CB | 10.16 | 26.75 | −8.91 | 10.01 |
| 89 | LEU | CG | 10.87 | 27.96 | −8.29 | 9.88 |
| 89 | LEU | CD1 | 11.40 | 27.62 | −6.89 | 9.84 |
| 89 | LEU | CD2 | 12.00 | 28.41 | −9.20 | 9.94 |
| 89 | LEU | C | 7.98 | 27.04 | −7.71 | 10.00 |
| 89 | LEU | O | 7.45 | 27.74 | −8.57 | 10.05 |
| 90 | VAL | N | 7.64 | 27.08 | −6.42 | 9.84 |
| 90 | VAL | CA | 6.55 | 27.91 | −5.94 | 9.76 |
| 90 | VAL | CB | 5.55 | 27.04 | −5.14 | 9.72 |
| 90 | VAL | CG1 | 4.36 | 27.87 | −4.68 | 9.84 |
| 90 | VAL | CG2 | 5.10 | 25.86 | −5.99 | 9.91 |
| 90 | VAL | C | 6.96 | 29.06 | −5.03 | 9.74 |
| 90 | VAL | O | 7.82 | 28.90 | −4.17 | 9.70 |
| 91 | ASN | N | 6.36 | 30.22 | −5.24 | 9.72 |
| 91 | ASN | CA | 6.61 | 31.36 | −4.37 | 9.77 |
| 91 | ASN | CB | 7.19 | 32.57 | −5.09 | 10.13 |
| 91 | ASN | CG | 7.36 | 33.75 | −4.15 | 10.36 |
| 91 | ASN | OD1 | 6.42 | 34.52 | −3.93 | 10.60 |
| 91 | ASN | ND2 | 8.54 | 33.87 | −3.55 | 10.68 |
| 91 | ASN | C | 5.28 | 31.76 | −3.75 | 9.66 |
| 91 | ASN | O | 4.28 | 31.95 | −4.45 | 9.62 |
| 92 | ILE | N | 5.28 | 31.86 | −2.43 | 9.49 |
| 92 | ILE | CA | 4.09 | 32.26 | −1.69 | 9.43 |
| 92 | ILE | CB | 3.70 | 31.20 | −0.64 | 9.45 |
| 92 | ILE | CG2 | 2.52 | 31.69 | 0.19 | 9.51 |
| 92 | ILE | CG1 | 3.39 | 29.87 | −1.34 | 9.57 |
| 92 | ILE | CD1 | 3.01 | 28.75 | −0.40 | 9.63 |
| 92 | ILE | C | 4.46 | 33.57 | −1.01 | 9.40 |
| 92 | ILE | O | 5.45 | 33.62 | −0.26 | 9.28 |
| 93 | ASP | N | 3.69 | 34.62 | −1.29 | 9.42 |
| 93 | ASP | CA | 3.94 | 35.94 | −0.72 | 9.50 |
| 93 | ASP | CB | 4.15 | 36.97 | −1.83 | 9.83 |
| 93 | ASP | CG | 4.77 | 38.27 | −1.31 | 10.11 |
| 93 | ASP | OD1 | 4.26 | 38.80 | −0.30 | 10.33 |
| 93 | ASP | OD2 | 5.75 | 38.77 | −1.91 | 10.66 |
| 93 | ASP | C | 2.73 | 36.30 | 0.12 | 9.36 |
| 93 | ASP | O | 1.62 | 36.46 | −0.40 | 9.24 |
| 94 | VAL | N | 2.95 | 36.45 | 1.43 | 9.26 |
| 94 | VAL | CA | 1.89 | 36.75 | 2.39 | 9.38 |
| 94 | VAL | CB | 1.89 | 35.71 | 3.55 | 9.33 |
| 94 | VAL | CG1 | 0.73 | 35.96 | 4.50 | 9.37 |
| 94 | VAL | CG2 | 1.83 | 34.29 | 2.99 | 9.33 |
| 94 | VAL | C | 2.07 | 38.14 | 3.00 | 9.40 |
| 94 | VAL | O | 3.07 | 38.40 | 3.66 | 9.40 |
| 95 | SER | N | 1.10 | 39.02 | 2.78 | 9.48 |
| 95 | SER | CA | 1.15 | 40.36 | 3.36 | 9.63 |
| 95 | SER | CB | 1.57 | 41.40 | 2.31 | 9.71 |
| 95 | SER | OG | 0.65 | 41.48 | 1.24 | 9.65 |
| 95 | SER | C | −0.23 | 40.66 | 3.92 | 9.78 |
| 95 | SER | O | −1.24 | 40.26 | 3.34 | 9.80 |
| 96 | ALA | N | −0.29 | 41.35 | 5.05 | 9.93 |
| 96 | ALA | CA | −1.57 | 41.64 | 5.68 | 10.12 |
| 96 | ALA | CB | −2.10 | 40.38 | 6.37 | 10.17 |
| 96 | ALA | C | −1.44 | 42.76 | 6.68 | 10.29 |
| 96 | ALA | O | −0.33 | 43.24 | 6.97 | 10.36 |
| 97 | LEU | N | −2.57 | 43.18 | 7.22 | 10.52 |
| 97 | LEU | CA | −2.59 | 44.24 | 8.21 | 10.71 |
| 97 | LEU | CB | −3.04 | 45.57 | 7.58 | 10.77 |
| 97 | LEU | CG | −4.43 | 45.63 | 6.92 | 10.80 |
| 97 | LEU | CD1 | −4.93 | 47.07 | 6.96 | 10.92 |
| 97 | LEU | CD2 | −4.35 | 45.12 | 5.48 | 10.83 |
| 97 | LEU | C | −3.52 | 43.89 | 9.36 | 10.87 |
| 97 | LEU | O | −4.51 | 43.17 | 9.18 | 10.82 |
| 98 | LYS | N | −3.18 | 44.38 | 10.54 | 10.99 |
| 98 | LYS | CA | −3.99 | 44.18 | 11.74 | 11.29 |
| 98 | LYS | CB | −3.55 | 42.95 | 12.54 | 11.30 |
| 98 | LYS | CG | −4.33 | 42.76 | 13.84 | 11.32 |
| 98 | LYS | CD | −4.04 | 41.42 | 14.49 | 11.31 |
| 98 | LYS | CE | −4.74 | 41.28 | 15.84 | 11.41 |
| 98 | LYS | NZ | −4.57 | 39.91 | 16.40 | 11.40 |
| 98 | LYS | C | −3.82 | 45.44 | 12.58 | 11.52 |
| 98 | LYS | O | −2.70 | 45.87 | 12.84 | 11.52 |
| 99 | ASN | N | −4.94 | 46.03 | 13.00 | 11.80 |
| 99 | ASN | CA | −4.92 | 47.25 | 13.80 | 12.02 |
| 99 | ASN | CB | −4.39 | 46.96 | 15.21 | 12.33 |
| 99 | ASN | CG | −5.30 | 46.02 | 15.99 | 12.49 |
| 99 | ASN | OD1 | −6.52 | 46.14 | 15.92 | 12.87 |
| 99 | ASN | ND2 | −4.70 | 45.10 | 16.73 | 12.66 |
| 99 | ASN | C | −4.09 | 48.35 | 13.13 | 11.93 |
| 99 | ASN | O | −3.41 | 49.13 | 13.81 | 12.16 |
| 100 | GLY | N | −4.15 | 48.40 | 11.80 | 11.86 |
| 100 | GLY | CA | −3.44 | 49.43 | 11.07 | 11.71 |
| 100 | GLY | C | −1.99 | 49.20 | 10.73 | 11.55 |
| 100 | GLY | O | −1.39 | 50.02 | 10.03 | 11.62 |
| 101 | TYR | N | −1.40 | 48.11 | 11.21 | 11.34 |
| 101 | TYR | CA | 0.00 | 47.84 | 10.94 | 11.09 |
| 101 | TYR | CB | 0.76 | 47.64 | 12.25 | 11.18 |
| 101 | TYR | CG | 0.81 | 48.91 | 13.06 | 11.36 |
| 101 | TYR | CD1 | 1.84 | 49.84 | 12.88 | 11.49 |
| 101 | TYR | CE1 | 1.85 | 51.05 | 13.56 | 11.75 |
| 101 | TYR | CD2 | −0.22 | 49.24 | 13.94 | 11.52 |
| 101 | TYR | CE2 | −0.22 | 50.45 | 14.63 | 11.70 |
| 101 | TYR | CZ | 0.81 | 51.35 | 14.43 | 11.74 |
| 101 | TYR | OH | 0.81 | 52.56 | 15.11 | 12.10 |
| 101 | TYR | C | 0.17 | 46.65 | 10.00 | 10.85 |
| 101 | TYR | O | −0.61 | 45.69 | 10.04 | 10.76 |
| 102 | TYR | N | 1.21 | 46.72 | 9.18 | 10.63 |
| 102 | TYR | CA | 1.48 | 45.71 | 8.16 | 10.42 |
| 102 | TYR | CB | 1.62 | 46.38 | 6.80 | 10.54 |
| 102 | TYR | CG | 0.36 | 46.98 | 6.22 | 10.69 |
| 102 | TYR | CD1 | −0.29 | 48.04 | 6.86 | 10.80 |
| 102 | TYR | CE1 | −1.42 | 48.63 | 6.28 | 10.97 |
| 102 | TYR | CD2 | −0.14 | 46.53 | 5.00 | 10.79 |
| 102 | TYR | CE2 | −1.25 | 47.11 | 4.43 | 10.92 |
| 102 | TYR | CZ | −1.89 | 48.16 | 5.06 | 10.96 |
| 102 | TYR | OH | −2.97 | 48.75 | 4.46 | 11.17 |
| 102 | TYR | C | 2.72 | 44.84 | 8.33 | 10.28 |
| 102 | TYR | O | 3.67 | 45.20 | 9.02 | 10.13 |
| 103 | ALA | N | 2.69 | 43.71 | 7.63 | 10.05 |
| 103 | ALA | CA | 3.80 | 42.77 | 7.57 | 9.86 |
| 103 | ALA | CB | 3.60 | 41.61 | 8.54 | 9.99 |
| 103 | ALA | C | 3.81 | 42.27 | 6.13 | 9.73 |
| 103 | ALA | O | 2.77 | 42.24 | 5.47 | 9.61 |
| 104 | ASP | N | 4.97 | 41.86 | 5.65 | 9.45 |
| 104 | ASP | CA | 5.12 | 41.39 | 4.28 | 9.35 |
| 104 | ASP | CB | 5.49 | 42.59 | 3.40 | 9.41 |
| 104 | ASP | CG | 5.56 | 42.27 | 1.93 | 9.50 |
| 104 | ASP | OD1 | 5.16 | 41.16 | 1.51 | 9.51 |
| 104 | ASP | OD2 | 6.03 | 43.15 | 1.18 | 9.58 |
| 104 | ASP | C | 6.24 | 40.36 | 4.29 | 9.25 |
| 104 | ASP | O | 7.37 | 40.65 | 4.69 | 9.20 |
| 105 | THR | N | 5.93 | 39.14 | 3.87 | 9.15 |
| 105 | THR | CA | 6.94 | 38.09 | 3.85 | 9.09 |
| 105 | THR | CB | 7.01 | 37.38 | 5.22 | 8.97 |
| 105 | THR | OG1 | 8.08 | 36.43 | 5.22 | 8.87 |
| 105 | THR | CG2 | 5.69 | 36.69 | 5.54 | 9.03 |
| 105 | THR | C | 6.61 | 37.10 | 2.74 | 9.14 |
| 105 | THR | O | 5.47 | 37.01 | 2.29 | 9.17 |
| 106 | GLY | N | 7.62 | 36.38 | 2.27 | 9.08 |
| 106 | GLY | CA | 7.40 | 35.43 | 1.20 | 9.10 |
| 106 | GLY | C | 8.54 | 34.44 | 1.09 | 8.98 |
| 106 | GLY | O | 9.63 | 34.67 | 1.62 | 8.92 |
| 107 | ILE | N | 8.29 | 33.35 | 0.39 | 8.93 |
| 107 | ILE | CA | 9.31 | 32.33 | 0.23 | 8.93 |
| 107 | ILE | CB | 9.35 | 31.43 | 1.50 | 9.03 |
| 107 | ILE | CG2 | 8.01 | 30.73 | 1.69 | 9.00 |
| 107 | ILE | CG1 | 10.47 | 30.40 | 1.41 | 9.11 |
| 107 | ILE | CD1 | 10.87 | 29.82 | 2.76 | 9.42 |
| 107 | ILE | C | 9.10 | 31.47 | −1.01 | 8.91 |
| 107 | ILE | O | 7.97 | 31.12 | −1.37 | 8.71 |
| 108 | SER | N | 10.21 | 31.15 | −1.67 | 8.97 |
| 108 | SER | CA | 10.20 | 30.29 | −2.85 | 9.06 |
| 108 | SER | CB | 11.04 | 30.88 | −3.99 | 9.02 |
| 108 | SER | OG | 10.43 | 32.02 | −4.56 | 8.97 |
| 108 | SER | C | 10.80 | 28.97 | −2.42 | 9.25 |
| 108 | SER | O | 11.76 | 28.93 | −1.65 | 9.17 |
| 109 | PHE | N | 10.23 | 27.88 | −2.92 | 9.39 |
| 109 | PHE | CA | 10.70 | 26.55 | −2.59 | 9.65 |
| 109 | PHE | CB | 10.09 | 26.07 | −1.26 | 9.56 |
| 109 | PHE | CG | 8.59 | 26.12 | −1.23 | 9.47 |
| 109 | PHE | CD1 | 7.92 | 27.31 | −0.97 | 9.45 |
| 109 | PHE | CD2 | 7.84 | 24.98 | −1.49 | 9.47 |
| 109 | PHE | CE1 | 6.53 | 27.36 | −0.97 | 9.45 |
| 109 | PHE | CE2 | 6.45 | 25.02 | −1.50 | 9.43 |
| 109 | PHE | CZ | 5.79 | 26.22 | −1.23 | 9.38 |
| 109 | PHE | C | 10.32 | 25.58 | −3.71 | 9.85 |
| 109 | PHE | O | 9.48 | 25.89 | −4.56 | 9.84 |
| 110 | VAL | N | 10.95 | 24.41 | −3.69 | 10.15 |
| 110 | VAL | CA | 10.70 | 23.38 | −4.68 | 10.42 |
| 110 | VAL | CB | 12.01 | 22.70 | −5.11 | 10.42 |
| 110 | VAL | CG1 | 11.73 | 21.56 | −6.08 | 10.42 |
| 110 | VAL | CG2 | 12.93 | 23.73 | −5.75 | 10.42 |
| 110 | VAL | C | 9.78 | 22.33 | −4.08 | 10.69 |
| 110 | VAL | O | 9.97 | 21.91 | −2.93 | 10.70 |
| 111 | VAL | N | 8.77 | 21.91 | −4.84 | 11.04 |
| 111 | VAL | CA | 7.85 | 20.88 | −4.37 | 11.45 |
| 111 | VAL | CB | 6.47 | 21.00 | −5.05 | 11.42 |
| 111 | VAL | CG1 | 5.55 | 19.88 | −4.58 | 11.38 |
| 111 | VAL | CG2 | 5.85 | 22.35 | −4.73 | 11.40 |
| 111 | VAL | C | 8.48 | 19.54 | −4.75 | 11.82 |
| 111 | VAL | O | 8.64 | 19.24 | −5.93 | 11.87 |
| 112 | GLY | N | 8.84 | 18.75 | −3.75 | 12.25 |
| 112 | GLY | CA | 9.46 | 17.46 | −4.03 | 12.82 |
| 112 | GLY | C | 10.77 | 17.59 | −4.76 | 13.15 |
| 112 | GLY | O | 11.66 | 18.33 | −4.34 | 13.32 |
| 113 | GLU | N | 10.91 | 16.87 | −5.87 | 13.57 |
| 113 | GLU | CA | 12.14 | 16.92 | −6.65 | 13.87 |
| 113 | GLU | CB | 12.83 | 15.55 | −6.66 | 14.12 |
| 113 | GLU | CG | 13.10 | 14.97 | −5.29 | 14.61 |
| 113 | GLU | CD | 13.60 | 13.55 | −5.38 | 14.82 |
| 113 | GLU | OE1 | 12.95 | 12.72 | −6.05 | 15.15 |
| 113 | GLU | OE2 | 14.65 | 13.25 | −4.77 | 15.06 |
| 113 | GLU | C | 11.89 | 17.32 | −8.09 | 13.94 |
| 113 | GLU | O | 10.91 | 16.89 | −8.70 | 13.95 |
| 114 | SER | N | 12.79 | 18.14 | −8.62 | 14.05 |
| 114 | SER | CA | 12.74 | 18.58 | −10.01 | 14.18 |
| 114 | SER | CB | 12.99 | 20.08 | −10.12 | 14.18 |
| 114 | SER | OG | 13.23 | 20.45 | −11.47 | 14.21 |
| 114 | SER | C | 13.88 | 17.84 | −10.68 | 14.29 |
| 114 | SER | O | 14.81 | 17.39 | −10.01 | 14.31 |
| 115 | ASP | N | 13.82 | 17.71 | −12.00 | 14.40 |
| 115 | ASP | CA | 14.90 | 17.03 | −12.71 | 14.52 |
| 115 | ASP | CB | 14.40 | 16.53 | −14.07 | 15.02 |
| 115 | ASP | CG | 14.08 | 17.66 | −15.03 | 15.42 |
| 115 | ASP | OD1 | 13.48 | 18.66 | −14.59 | 15.80 |
| 115 | ASP | OD2 | 14.43 | 17.54 | −16.22 | 15.84 |
| 115 | ASP | C | 16.05 | 18.02 | −12.91 | 14.33 |
| 115 | ASP | O | 17.16 | 17.63 | −13.30 | 14.31 |
| 116 | ASP | N | 15.79 | 19.29 | −12.62 | 14.08 |
| 116 | ASP | CA | 16.81 | 20.33 | −12.77 | 13.88 |
| 116 | ASP | CB | 16.28 | 21.45 | −13.68 | 13.95 |
| 116 | ASP | CG | 17.37 | 22.38 | −14.15 | 14.02 |
| 116 | ASP | OD1 | 18.42 | 22.47 | −13.48 | 14.02 |
| 116 | ASP | OD2 | 17.18 | 23.04 | −15.20 | 14.16 |
| 116 | ASP | C | 17.18 | 20.92 | −11.42 | 13.67 |
| 116 | ASP | O | 16.37 | 21.59 | −10.78 | 13.62 |
| 117 | PRO | N | 18.42 | 20.68 | −10.96 | 13.47 |
| 117 | PRO | CD | 19.47 | 19.84 | −11.56 | 13.45 |
| 117 | PRO | CA | 18.84 | 21.21 | −9.66 | 13.36 |
| 117 | PRO | CB | 20.20 | 20.54 | −9.44 | 13.44 |
| 117 | PRO | CG | 20.70 | 20.30 | −10.84 | 13.48 |
| 117 | PRO | C | 18.91 | 22.74 | −9.59 | 13.19 |
| 117 | PRO | O | 19.11 | 23.30 | −8.52 | 13.12 |
| 118 | MET | N | 18.75 | 23.40 | −10.73 | 13.10 |
| 118 | MET | CA | 18.79 | 24.86 | −10.76 | 12.99 |
| 118 | MET | CB | 18.75 | 25.37 | −12.20 | 13.33 |
| 118 | MET | CG | 18.67 | 26.89 | −12.33 | 13.66 |
| 118 | MET | SD | 20.13 | 27.78 | −11.71 | 14.22 |
| 118 | MET | CE | 21.24 | 27.60 | −13.09 | 14.15 |
| 118 | MET | C | 17.63 | 25.48 | −9.97 | 12.75 |
| 118 | MET | O | 17.75 | 26.58 | −9.44 | 12.67 |
| 119 | LYS | N | 16.50 | 24.78 | −9.91 | 12.46 |
| 119 | LYS | CA | 15.37 | 25.31 | −9.17 | 12.15 |
| 119 | LYS | CB | 14.14 | 24.40 | −9.33 | 12.11 |
| 119 | LYS | CG | 13.47 | 24.55 | −10.69 | 12.12 |
| 119 | LYS | CD | 12.18 | 23.73 | −10.77 | 12.06 |
| 119 | LYS | CE | 11.36 | 24.11 | −12.00 | 12.16 |
| 119 | LYS | NZ | 10.15 | 23.24 | −12.16 | 12.16 |
| 119 | LYS | C | 15.72 | 25.46 | −7.69 | 11.99 |
| 119 | LYS | O | 15.46 | 26.51 | −7.09 | 11.92 |
| 120 | GLN | N | 16.33 | 24.44 | −7.10 | 11.85 |
| 120 | GLN | CA | 16.72 | 24.53 | −5.70 | 11.67 |
| 120 | GLN | CB | 17.13 | 23.15 | −5.17 | 11.72 |
| 120 | GLN | CG | 17.50 | 23.13 | −3.68 | 11.77 |
| 120 | GLN | CD | 16.38 | 23.64 | −2.78 | 11.84 |
| 120 | GLN | OE1 | 15.24 | 23.16 | −2.85 | 12.03 |
| 120 | GLN | NE2 | 16.70 | 24.60 | −1.92 | 11.96 |
| 120 | GLN | C | 17.89 | 25.51 | −5.55 | 11.53 |
| 120 | GLN | O | 17.98 | 26.22 | −4.55 | 11.45 |
| 121 | LYS | N | 18.77 | 25.57 | −6.54 | 11.39 |
| 121 | LYS | CA | 19.91 | 26.48 | −6.47 | 11.33 |
| 121 | LYS | CB | 20.79 | 26.39 | −7.72 | 11.49 |
| 121 | LYS | CG | 22.00 | 27.32 | −7.64 | 11.69 |
| 121 | LYS | CD | 22.94 | 27.20 | −8.82 | 11.96 |
| 121 | LYS | CE | 24.11 | 28.16 | −8.68 | 12.14 |
| 121 | LYS | NZ | 25.10 | 28.03 | −9.79 | 12.40 |
| 121 | LYS | C | 19.48 | 27.92 | −6.28 | 11.21 |
| 121 | LYS | O | 20.03 | 28.61 | −5.43 | 11.17 |
| 122 | VAL | N | 18.53 | 28.39 | −7.08 | 11.11 |
| 122 | VAL | CA | 18.11 | 29.78 | −6.92 | 10.87 |
| 122 | VAL | CB | 17.15 | 30.25 | −8.05 | 10.88 |
| 122 | VAL | CG1 | 17.89 | 30.28 | −9.37 | 10.86 |
| 122 | VAL | CG2 | 15.93 | 29.34 | −8.14 | 10.78 |
| 122 | VAL | C | 17.48 | 30.01 | −5.55 | 10.85 |
| 122 | VAL | O | 17.56 | 31.12 | −5.01 | 10.89 |
| 123 | CYS | N | 16.85 | 28.98 | −4.99 | 10.78 |
| 123 | CYS | CA | 16.26 | 29.14 | −3.66 | 10.79 |
| 123 | CYS | CB | 15.38 | 27.93 | −3.30 | 10.68 |
| 123 | CYS | SG | 13.86 | 27.79 | −4.26 | 10.46 |
| 123 | CYS | C | 17.39 | 29.27 | −2.65 | 10.96 |
| 123 | CYS | O | 17.32 | 30.10 | −1.75 | 10.92 |
| 124 | ASP | N | 18.41 | 28.43 | −2.79 | 11.15 |
| 124 | ASP | CA | 19.55 | 28.46 | −1.88 | 11.39 |
| 124 | ASP | CB | 20.59 | 27.38 | −2.23 | 11.57 |
| 124 | ASP | CG | 20.09 | 25.97 | −1.99 | 11.81 |
| 124 | ASP | OD1 | 19.15 | 25.78 | −1.18 | 12.07 |
| 124 | ASP | OD2 | 20.65 | 25.04 | −2.60 | 12.04 |
| 124 | ASP | C | 20.25 | 29.82 | −1.94 | 11.44 |
| 124 | ASP | O | 20.59 | 30.40 | −0.91 | 11.41 |
| 125 | VAL | N | 20.45 | 30.34 | −3.14 | 11.56 |
| 125 | VAL | CA | 21.14 | 31.61 | −3.27 | 11.72 |
| 125 | VAL | CB | 21.66 | 31.79 | −4.72 | 11.69 |
| 125 | VAL | CG1 | 22.36 | 33.14 | −4.85 | 11.64 |
| 125 | VAL | CG2 | 22.65 | 30.67 | −5.05 | 11.67 |
| 125 | VAL | C | 20.29 | 32.80 | −2.84 | 11.93 |
| 125 | VAL | O | 20.83 | 33.80 | −2.37 | 11.95 |
| 126 | ALA | N | 18.97 | 32.70 | −2.99 | 12.11 |
| 126 | ALA | CA | 18.09 | 33.78 | −2.56 | 12.30 |
| 126 | ALA | CB | 16.64 | 33.50 | −3.00 | 12.35 |
| 126 | ALA | C | 18.17 | 33.90 | −1.04 | 12.50 |
| 126 | ALA | O | 18.11 | 34.99 | −0.48 | 12.39 |
| 127 | THR | N | 18.28 | 32.75 | −0.38 | 12.73 |
| 127 | THR | CA | 18.38 | 32.72 | 1.07 | 13.08 |
| 127 | THR | CB | 18.28 | 31.28 | 1.59 | 13.23 |
| 127 | THR | OG1 | 17.09 | 30.68 | 1.07 | 13.57 |
| 127 | THR | CG2 | 18.21 | 31.26 | 3.11 | 13.41 |
| 127 | THR | C | 19.71 | 33.35 | 1.49 | 13.18 |
| 127 | THR | O | 19.76 | 34.15 | 2.41 | 13.11 |
| 128 | MET | N | 20.78 | 32.98 | 0.80 | 13.36 |
| 128 | MET | CA | 22.09 | 33.55 | 1.10 | 13.56 |
| 128 | MET | CB | 23.19 | 32.87 | 0.28 | 14.16 |
| 128 | MET | CG | 23.58 | 31.48 | 0.77 | 14.95 |
| 128 | MET | SD | 25.11 | 30.89 | 0.01 | 15.92 |
| 128 | MET | CE | 24.54 | 30.60 | −1.63 | 15.74 |
| 128 | MET | C | 22.09 | 35.04 | 0.82 | 13.36 |
| 128 | MET | O | 22.67 | 35.83 | 1.56 | 13.21 |
| 129 | ALA | N | 21.41 | 35.44 | −0.26 | 13.16 |
| 129 | ALA | CA | 21.36 | 36.85 | −0.61 | 13.03 |
| 129 | ALA | CB | 20.63 | 37.04 | −1.94 | 12.96 |
| 129 | ALA | C | 20.66 | 37.65 | 0.49 | 12.99 |
| 129 | ALA | O | 21.09 | 38.76 | 0.82 | 12.90 |
| 130 | PHE | N | 19.59 | 37.10 | 1.06 | 13.04 |
| 130 | PHE | CA | 18.89 | 37.81 | 2.12 | 13.10 |
| 130 | PHE | CB | 17.60 | 37.08 | 2.52 | 12.86 |
| 130 | PHE | CG | 16.87 | 37.74 | 3.65 | 12.61 |
| 130 | PHE | CD1 | 16.01 | 38.81 | 3.42 | 12.52 |
| 130 | PHE | CD2 | 17.12 | 37.38 | 4.97 | 12.50 |
| 130 | PHE | CE1 | 15.42 | 39.51 | 4.47 | 12.40 |
| 130 | PHE | CE2 | 16.54 | 38.07 | 6.03 | 12.41 |
| 130 | PHE | CZ | 15.69 | 39.14 | 5.78 | 12.37 |
| 130 | PHE | C | 19.77 | 37.97 | 3.36 | 13.34 |
| 130 | PHE | O | 19.84 | 39.05 | 3.94 | 13.23 |
| 131 | GLU | N | 20.44 | 36.89 | 3.75 | 13.72 |
| 131 | GLU | CA | 21.33 | 36.91 | 4.91 | 14.11 |
| 131 | GLU | CB | 21.96 | 35.52 | 5.12 | 14.41 |
| 131 | GLU | CG | 20.96 | 34.39 | 5.28 | 15.11 |
| 131 | GLU | CD | 21.63 | 33.02 | 5.32 | 15.48 |
| 131 | GLU | OE1 | 22.63 | 32.81 | 4.59 | 15.86 |
| 131 | GLU | OE2 | 21.16 | 32.14 | 6.08 | 15.91 |
| 131 | GLU | C | 22.45 | 37.92 | 4.71 | 14.18 |
| 131 | GLU | O | 22.82 | 38.64 | 5.64 | 14.14 |
| 132 | ASN | N | 23.00 | 37.97 | 3.50 | 14.33 |
| 132 | ASN | CA | 24.08 | 38.90 | 3.23 | 14.54 |
| 132 | ASN | CB | 24.74 | 38.57 | 1.89 | 14.71 |
| 132 | ASN | CG | 25.51 | 37.26 | 1.94 | 14.92 |
| 132 | ASN | OD1 | 25.94 | 36.82 | 3.01 | 15.13 |
| 132 | ASN | ND2 | 25.71 | 36.65 | 0.78 | 15.08 |
| 132 | ASN | C | 23.56 | 40.33 | 3.23 | 14.63 |
| 132 | ASN | O | 24.26 | 41.26 | 3.65 | 14.57 |
| 133 | ALA | N | 22.33 | 40.51 | 2.77 | 14.73 |
| 133 | ALA | CA | 21.73 | 41.84 | 2.71 | 14.96 |
| 133 | ALA | CB | 20.41 | 41.77 | 1.95 | 14.88 |
| 133 | ALA | C | 21.50 | 42.44 | 4.10 | 15.11 |
| 133 | ALA | O | 21.75 | 43.63 | 4.30 | 15.14 |
| 134 | ILE | N | 21.02 | 41.64 | 5.05 | 15.31 |
| 134 | ILE | CA | 20.77 | 42.19 | 6.38 | 15.60 |
| 134 | ILE | CB | 19.58 | 41.50 | 7.07 | 15.63 |
| 134 | ILE | CG2 | 18.31 | 41.74 | 6.28 | 15.66 |
| 134 | ILE | CG1 | 19.87 | 40.01 | 7.24 | 15.65 |
| 134 | ILE | CD1 | 18.95 | 39.34 | 8.24 | 15.69 |
| 134 | ILE | C | 21.95 | 42.13 | 7.33 | 15.79 |
| 134 | ILE | O | 21.90 | 42.69 | 8.42 | 15.78 |
| 135 | ALA | N | 23.03 | 41.48 | 6.90 | 16.11 |
| 135 | ALA | CA | 24.22 | 41.30 | 7.73 | 16.52 |
| 135 | ALA | CB | 25.34 | 40.68 | 6.90 | 16.49 |
| 135 | ALA | C | 24.74 | 42.55 | 8.44 | 16.81 |
| 135 | ALA | O | 25.13 | 42.47 | 9.61 | 16.98 |
| 136 | LYS | N | 24.75 | 43.68 | 7.75 | 17.13 |
| 136 | LYS | CA | 25.26 | 44.91 | 8.36 | 17.41 |
| 136 | LYS | CB | 26.53 | 45.36 | 7.62 | 17.59 |
| 136 | LYS | CG | 27.72 | 44.41 | 7.79 | 17.84 |
| 136 | LYS | CD | 28.93 | 44.85 | 6.97 | 18.06 |
| 136 | LYS | CE | 28.67 | 44.74 | 5.47 | 18.20 |
| 136 | LYS | NZ | 29.85 | 45.22 | 4.68 | 18.33 |
| 136 | LYS | C | 24.26 | 46.06 | 8.43 | 17.49 |
| 136 | LYS | O | 24.64 | 47.20 | 8.72 | 17.54 |
| 137 | VAL | N | 22.99 | 45.77 | 8.16 | 17.52 |
| 137 | VAL | CA | 21.94 | 46.79 | 8.22 | 17.53 |
| 137 | VAL | CB | 20.55 | 46.18 | 7.90 | 17.59 |
| 137 | VAL | CG1 | 19.45 | 47.17 | 8.24 | 17.73 |
| 137 | VAL | CG2 | 20.47 | 45.80 | 6.43 | 17.66 |
| 137 | VAL | C | 21.92 | 47.41 | 9.61 | 17.49 |
| 137 | VAL | O | 21.78 | 46.71 | 10.61 | 17.50 |
| 138 | LYS | N | 22.09 | 48.73 | 9.66 | 17.43 |
| 138 | LYS | CA | 22.10 | 49.45 | 10.92 | 17.30 |
| 138 | LYS | CB | 23.45 | 49.32 | 11.63 | 17.56 |
| 138 | LYS | CG | 23.50 | 48.29 | 12.74 | 17.88 |
| 138 | LYS | CD | 24.80 | 48.40 | 13.51 | 18.14 |
| 138 | LYS | CE | 24.89 | 47.36 | 14.62 | 18.30 |
| 138 | LYS | NZ | 24.99 | 45.98 | 14.07 | 18.42 |
| 138 | LYS | C | 21.83 | 50.92 | 10.66 | 17.07 |
| 138 | LYS | O | 21.90 | 51.38 | 9.51 | 17.06 |
| 139 | PRO | N | 21.52 | 51.69 | 11.71 | 16.83 |
| 139 | PRO | CD | 21.28 | 51.30 | 13.11 | 16.81 |
| 139 | PRO | CA | 21.26 | 53.12 | 11.51 | 16.62 |
| 139 | PRO | CB | 21.03 | 53.63 | 12.93 | 16.68 |
| 139 | PRO | CG | 20.43 | 52.45 | 13.62 | 16.74 |
| 139 | PRO | C | 22.47 | 53.79 | 10.85 | 16.40 |
| 139 | PRO | O | 23.61 | 53.49 | 11.19 | 16.42 |
| 140 | GLY | N | 22.20 | 54.67 | 9.89 | 16.12 |
| 140 | GLY | CA | 23.27 | 55.38 | 9.20 | 15.77 |
| 140 | GLY | C | 23.88 | 54.71 | 7.99 | 15.59 |
| 140 | GLY | O | 24.61 | 55.35 | 7.22 | 15.54 |
| 141 | THR | N | 23.60 | 53.42 | 7.79 | 15.41 |
| 141 | THR | CA | 24.16 | 52.70 | 6.65 | 15.29 |
| 141 | THR | CB | 24.15 | 51.17 | 6.87 | 15.35 |
| 141 | THR | OG1 | 22.80 | 50.72 | 7.05 | 15.48 |
| 141 | THR | CG2 | 24.96 | 50.81 | 8.10 | 15.45 |
| 141 | THR | C | 23.40 | 53.03 | 5.38 | 15.09 |
| 141 | THR | O | 22.23 | 53.44 | 5.43 | 15.12 |
| 142 | LYS | N | 24.05 | 52.84 | 4.24 | 14.95 |
| 142 | LYS | CA | 23.44 | 53.14 | 2.96 | 14.75 |
| 142 | LYS | CB | 24.52 | 53.28 | 1.89 | 14.93 |
| 142 | LYS | CG | 25.47 | 54.44 | 2.13 | 15.11 |
| 142 | LYS | CD | 26.56 | 54.50 | 1.08 | 15.37 |
| 142 | LYS | CE | 27.51 | 55.66 | 1.36 | 15.59 |
| 142 | LYS | NZ | 28.61 | 55.74 | 0.37 | 15.89 |
| 142 | LYS | C | 22.43 | 52.10 | 2.51 | 14.61 |
| 142 | LYS | O | 22.69 | 50.90 | 2.57 | 14.49 |
| 143 | LEU | N | 21.28 | 52.58 | 2.06 | 14.42 |
| 143 | LEU | CA | 20.22 | 51.71 | 1.58 | 14.29 |
| 143 | LEU | CB | 19.05 | 52.56 | 1.08 | 14.30 |
| 143 | LEU | CG | 17.87 | 51.84 | 0.43 | 14.31 |
| 143 | LEU | CD1 | 17.18 | 50.96 | 1.45 | 14.40 |
| 143 | LEU | CD2 | 16.89 | 52.86 | −0.15 | 14.36 |
| 143 | LEU | C | 20.78 | 50.86 | 0.44 | 14.23 |
| 143 | LEU | O | 20.48 | 49.67 | 0.34 | 14.11 |
| 144 | SER | N | 21.60 | 51.48 | −0.40 | 14.20 |
| 144 | SER | CA | 22.19 | 50.79 | −1.54 | 14.18 |
| 144 | SER | CB | 23.02 | 51.75 | −2.40 | 14.24 |
| 144 | SER | OG | 24.14 | 52.24 | −1.68 | 14.40 |
| 144 | SER | C | 23.05 | 49.58 | −1.16 | 14.11 |
| 144 | SER | O | 23.36 | 48.76 | −2.02 | 14.11 |
| 145 | ASN | N | 23.43 | 49.47 | 0.12 | 14.07 |
| 145 | ASN | CA | 24.23 | 48.32 | 0.56 | 13.98 |
| 145 | ASN | CB | 24.61 | 48.41 | 2.04 | 14.22 |
| 145 | ASN | CG | 25.64 | 49.47 | 2.32 | 14.40 |
| 145 | ASN | OD1 | 26.42 | 49.84 | 1.45 | 14.65 |
| 145 | ASN | ND2 | 25.67 | 49.93 | 3.56 | 14.66 |
| 145 | ASN | C | 23.44 | 47.04 | 0.38 | 13.80 |
| 145 | ASN | O | 24.01 | 45.98 | 0.15 | 13.74 |
| 146 | ILE | N | 22.12 | 47.13 | 0.54 | 13.61 |
| 146 | ILE | CA | 21.26 | 45.97 | 0.40 | 13.44 |
| 146 | ILE | CB | 19.80 | 46.34 | 0.71 | 13.42 |
| 146 | ILE | CG2 | 18.88 | 45.13 | 0.47 | 13.41 |
| 146 | ILE | CG1 | 19.69 | 46.79 | 2.17 | 13.49 |
| 146 | ILE | CD1 | 18.31 | 47.25 | 2.58 | 13.54 |
| 146 | ILE | C | 21.37 | 45.41 | −1.01 | 13.38 |
| 146 | ILE | O | 21.73 | 44.25 | −1.20 | 13.28 |
| 147 | GLY | N | 21.07 | 46.23 | −2.01 | 13.31 |
| 147 | GLY | CA | 21.14 | 45.77 | −3.39 | 13.29 |
| 147 | GLY | C | 22.56 | 45.36 | −3.77 | 13.30 |
| 147 | GLY | O | 22.75 | 44.41 | −4.53 | 13.26 |
| 148 | LYS | N | 23.55 | 46.08 | −3.25 | 13.35 |
| 148 | LYS | CA | 24.93 | 45.75 | −3.56 | 13.38 |
| 148 | LYS | CB | 25.87 | 46.71 | −2.83 | 13.65 |
| 148 | LYS | CG | 27.32 | 46.63 | −3.29 | 14.00 |
| 148 | LYS | CD | 28.20 | 47.59 | −2.50 | 14.48 |
| 148 | LYS | CE | 29.53 | 47.79 | −3.18 | 14.80 |
| 148 | LYS | NZ | 29.39 | 48.54 | −4.46 | 15.27 |
| 148 | LYS | C | 25.22 | 44.31 | −3.11 | 13.30 |
| 148 | LYS | O | 25.80 | 43.52 | −3.85 | 13.22 |
| 149 | ALA | N | 24.80 | 43.99 | −1.89 | 13.20 |
| 149 | ALA | CA | 25.01 | 42.66 | −1.33 | 13.10 |
| 149 | ALA | CB | 24.61 | 42.65 | 0.14 | 13.05 |
| 149 | ALA | C | 24.23 | 41.58 | −2.08 | 13.12 |
| 149 | ALA | O | 24.75 | 40.49 | −2.33 | 13.03 |
| 150 | VAL | N | 22.98 | 41.88 | −2.43 | 13.21 |
| 150 | VAL | CA | 22.14 | 40.93 | −3.15 | 13.28 |
| 150 | VAL | CB | 20.71 | 41.49 | −3.39 | 13.27 |
| 150 | VAL | CG1 | 19.90 | 40.53 | −4.25 | 13.27 |
| 150 | VAL | CG2 | 20.02 | 41.70 | −2.05 | 13.27 |
| 150 | VAL | C | 22.75 | 40.58 | −4.50 | 13.38 |
| 150 | VAL | O | 22.89 | 39.40 | −4.84 | 13.30 |
| 151 | HIS | N | 23.14 | 41.59 | −5.27 | 13.61 |
| 151 | HIS | CA | 23.72 | 41.35 | −6.59 | 13.92 |
| 151 | HIS | CB | 23.84 | 42.67 | −7.36 | 14.15 |
| 151 | HIS | CG | 24.29 | 42.51 | −8.77 | 14.48 |
| 151 | HIS | CD2 | 23.59 | 42.40 | −9.92 | 14.64 |
| 151 | HIS | ND1 | 25.62 | 42.41 | −9.13 | 14.64 |
| 151 | HIS | CE1 | 25.71 | 42.25 | −10.44 | 14.72 |
| 151 | HIS | NE2 | 24.49 | 42.24 | −10.95 | 14.75 |
| 151 | HIS | C | 25.08 | 40.67 | −6.49 | 14.00 |
| 151 | HIS | O | 25.42 | 39.85 | −7.34 | 13.95 |
| 152 | ASN | N | 25.86 | 40.99 | −5.46 | 14.13 |
| 152 | ASN | CA | 27.17 | 40.37 | −5.31 | 14.31 |
| 152 | ASN | CB | 27.95 | 41.04 | −4.16 | 14.54 |
| 152 | ASN | CG | 29.35 | 40.48 | −4.02 | 14.84 |
| 152 | ASN | OD1 | 29.54 | 39.32 | −3.65 | 15.13 |
| 152 | ASN | ND2 | 30.36 | 41.30 | −4.31 | 15.00 |
| 152 | ASN | C | 27.00 | 38.88 | −5.04 | 14.24 |
| 152 | ASN | O | 27.73 | 38.05 | −5.59 | 14.22 |
| 153 | THR | N | 26.03 | 38.55 | −4.20 | 14.24 |
| 153 | THR | CA | 25.75 | 37.15 | −3.87 | 14.24 |
| 153 | THR | CB | 24.63 | 37.03 | −2.83 | 14.18 |
| 153 | THR | OG1 | 25.01 | 37.73 | −1.64 | 14.13 |
| 153 | THR | CG2 | 24.36 | 35.58 | −2.48 | 14.07 |
| 153 | THR | C | 25.34 | 36.39 | −5.13 | 14.38 |
| 153 | THR | O | 25.74 | 35.24 | −5.34 | 14.32 |
| 154 | ALA | N | 24.54 | 37.04 | −5.97 | 14.50 |
| 154 | ALA | CA | 24.10 | 36.40 | −7.21 | 14.67 |
| 154 | ALA | CB | 23.10 | 37.30 | −7.93 | 14.62 |
| 154 | ALA | C | 25.30 | 36.13 | −8.12 | 14.86 |
| 154 | ALA | O | 25.47 | 35.02 | −8.62 | 14.82 |
| 155 | ARG | N | 26.14 | 37.14 | −8.30 | 15.16 |
| 155 | ARG | CA | 27.32 | 37.01 | −9.14 | 15.47 |
| 155 | ARG | CB | 28.05 | 38.36 | −9.19 | 15.89 |
| 155 | ARG | CG | 28.79 | 38.62 | −10.48 | 16.59 |
| 155 | ARG | CD | 29.90 | 37.62 | −10.73 | 17.12 |
| 155 | ARG | NE | 30.41 | 37.74 | −12.09 | 17.64 |
| 155 | ARG | CZ | 31.42 | 37.03 | −12.58 | 17.80 |
| 155 | ARG | NH1 | 32.06 | 36.15 | −11.82 | 18.01 |
| 155 | ARG | NH2 | 31.79 | 37.20 | −13.85 | 17.97 |
| 155 | ARG | C | 28.27 | 35.92 | −8.65 | 15.43 |
| 155 | ARG | O | 28.85 | 35.17 | −9.45 | 15.42 |
| 156 | GLN | N | 28.45 | 35.83 | −7.33 | 15.42 |
| 156 | GLN | CA | 29.34 | 34.81 | −6.76 | 15.43 |
| 156 | GLN | CB | 29.45 | 34.98 | −5.25 | 15.62 |
| 156 | GLN | CG | 30.08 | 36.26 | −4.76 | 16.01 |
| 156 | GLN | CD | 30.31 | 36.20 | −3.27 | 16.19 |
| 156 | GLN | OE1 | 30.34 | 37.23 | −2.59 | 16.43 |
| 156 | GLN | NE2 | 30.48 | 34.99 | −2.74 | 16.35 |
| 156 | GLN | C | 28.85 | 33.40 | −7.02 | 15.30 |
| 156 | GLN | O | 29.57 | 32.43 | −6.80 | 15.29 |
| 157 | ASN | N | 27.61 | 33.28 | −7.47 | 15.18 |
| 157 | ASN | CA | 27.03 | 31.97 | −7.75 | 15.08 |
| 157 | ASN | CB | 25.88 | 31.69 | −6.78 | 15.09 |
| 157 | ASN | CG | 26.36 | 31.60 | −5.33 | 15.05 |
| 157 | ASN | OD1 | 26.33 | 32.58 | −4.58 | 15.18 |
| 157 | ASN | ND2 | 26.82 | 30.41 | −4.95 | 15.11 |
| 157 | ASN | C | 26.54 | 31.85 | −9.19 | 15.04 |
| 157 | ASN | O | 25.65 | 31.05 | −9.49 | 14.98 |
| 158 | ASP | N | 27.14 | 32.64 | −10.07 | 15.03 |
| 158 | ASP | CA | 26.80 | 32.63 | −11.49 | 15.04 |
| 158 | ASP | CB | 27.33 | 31.34 | −12.12 | 15.35 |
| 158 | ASP | CG | 28.81 | 31.16 | −11.89 | 15.66 |
| 158 | ASP | OD1 | 29.57 | 32.12 | −12.18 | 15.93 |
| 158 | ASP | OD2 | 29.23 | 30.08 | −11.42 | 15.97 |
| 158 | ASP | C | 25.32 | 32.79 | −11.78 | 14.89 |
| 158 | ASP | O | 24.78 | 32.17 | −12.70 | 14.88 |
| 159 | LEU | N | 24.66 | 33.65 | −11.01 | 14.64 |
| 159 | LEU | CA | 23.24 | 33.90 | −11.19 | 14.41 |
| 159 | LEU | CB | 22.45 | 33.36 | −9.99 | 14.31 |
| 159 | LEU | CG | 22.49 | 31.84 | −9.83 | 14.25 |
| 159 | LEU | CD1 | 21.85 | 31.43 | −8.52 | 14.17 |
| 159 | LEU | CD2 | 21.78 | 31.18 | −11.01 | 14.20 |
| 159 | LEU | C | 22.98 | 35.39 | −11.37 | 14.32 |
| 159 | LEU | O | 23.90 | 36.20 | −11.28 | 14.24 |
| 160 | LYS | N | 21.73 | 35.74 | −11.62 | 14.27 |
| 160 | LYS | CA | 21.33 | 37.13 | −11.84 | 14.17 |
| 160 | LYS | CB | 20.77 | 37.30 | −13.25 | 14.41 |
| 160 | LYS | CG | 21.71 | 36.92 | −14.38 | 14.73 |
| 160 | LYS | CD | 22.97 | 37.78 | −14.37 | 15.07 |
| 160 | LYS | CE | 23.86 | 37.50 | −15.58 | 15.31 |
| 160 | LYS | NZ | 24.21 | 36.06 | −15.70 | 15.60 |
| 160 | LYS | C | 20.26 | 37.54 | −10.84 | 14.04 |
| 160 | LYS | O | 19.80 | 36.74 | −10.04 | 13.91 |
| 161 | VAL | N | 19.88 | 38.81 | −10.90 | 13.89 |
| 161 | VAL | CA | 18.83 | 39.33 | −10.03 | 13.79 |
| 161 | VAL | CB | 19.37 | 40.43 | −9.08 | 13.77 |
| 161 | VAL | CG1 | 20.53 | 39.90 | −8.25 | 13.77 |
| 161 | VAL | CG2 | 19.81 | 41.65 | −9.88 | 13.85 |
| 161 | VAL | C | 17.74 | 39.93 | −10.91 | 13.70 |
| 161 | VAL | O | 17.98 | 40.27 | −12.07 | 13.75 |
| 162 | ILE | N | 16.53 | 40.05 | −10.37 | 13.61 |
| 162 | ILE | CA | 15.45 | 40.66 | −11.14 | 13.49 |
| 162 | ILE | CB | 14.06 | 40.12 | −10.74 | 13.43 |
| 162 | ILE | CG2 | 12.96 | 40.92 | −11.44 | 13.46 |
| 162 | ILE | CG1 | 13.94 | 38.64 | −11.12 | 13.40 |
| 162 | ILE | CD1 | 14.10 | 38.38 | −12.62 | 13.34 |
| 162 | ILE | C | 15.57 | 42.14 | −10.78 | 13.48 |
| 162 | ILE | O | 15.30 | 42.53 | −9.64 | 13.45 |
| 163 | LYS | N | 15.98 | 42.94 | −11.75 | 13.51 |
| 163 | LYS | CA | 16.20 | 44.36 | −11.53 | 13.57 |
| 163 | LYS | CB | 17.13 | 44.91 | −12.62 | 13.78 |
| 163 | LYS | CG | 18.49 | 44.24 | −12.65 | 14.09 |
| 163 | LYS | CD | 19.31 | 44.69 | −13.84 | 14.46 |
| 163 | LYS | CE | 20.74 | 44.22 | −13.72 | 14.73 |
| 163 | LYS | NZ | 21.43 | 44.89 | −12.59 | 15.10 |
| 163 | LYS | C | 14.96 | 45.23 | −11.44 | 13.51 |
| 163 | LYS | O | 14.97 | 46.25 | −10.75 | 13.48 |
| 164 | ASN | N | 13.88 | 44.83 | −12.11 | 13.44 |
| 164 | ASN | CA | 12.68 | 45.65 | −12.08 | 13.43 |
| 164 | ASN | CB | 12.08 | 45.79 | −13.49 | 13.23 |
| 164 | ASN | CG | 11.58 | 44.48 | −14.06 | 13.18 |
| 164 | ASN | OD1 | 12.05 | 43.41 | −13.69 | 12.97 |
| 164 | ASN | ND2 | 10.63 | 44.57 | −14.98 | 13.15 |
| 164 | ASN | C | 11.60 | 45.27 | −11.07 | 13.47 |
| 164 | ASN | O | 10.43 | 45.59 | −11.24 | 13.60 |
| 165 | LEU | N | 12.02 | 44.59 | −10.01 | 13.52 |
| 165 | LEU | CA | 11.14 | 44.24 | −8.90 | 13.53 |
| 165 | LEU | CB | 10.84 | 42.74 | −8.84 | 13.67 |
| 165 | LEU | CG | 9.85 | 42.24 | −9.91 | 13.75 |
| 165 | LEU | CD1 | 9.53 | 40.78 | −9.65 | 13.80 |
| 165 | LEU | CD2 | 8.58 | 43.09 | −9.88 | 13.79 |
| 165 | LEU | C | 11.96 | 44.68 | −7.69 | 13.48 |
| 165 | LEU | O | 13.15 | 44.40 | −7.61 | 13.55 |
| 166 | THR | N | 11.32 | 45.40 | −6.76 | 13.38 |
| 166 | THR | CA | 12.05 | 45.92 | −5.62 | 13.24 |
| 166 | THR | CB | 12.30 | 47.43 | −5.77 | 13.22 |
| 166 | THR | OG1 | 11.06 | 48.12 | −5.55 | 13.29 |
| 166 | THR | CG2 | 12.79 | 47.75 | −7.17 | 13.22 |
| 166 | THR | C | 11.37 | 45.74 | −4.28 | 13.12 |
| 166 | THR | O | 10.19 | 45.45 | −4.20 | 13.14 |
| 167 | GLY | N | 12.17 | 45.93 | −3.23 | 12.98 |
| 167 | GLY | CA | 11.68 | 45.87 | −1.87 | 12.90 |
| 167 | GLY | C | 11.07 | 47.23 | −1.61 | 12.88 |
| 167 | GLY | O | 11.03 | 48.08 | −2.51 | 12.88 |
| 168 | HIS | N | 10.63 | 47.49 | −0.38 | 12.79 |
| 168 | HIS | CA | 9.98 | 48.76 | −0.10 | 12.78 |
| 168 | HIS | CB | 8.62 | 48.76 | −0.78 | 12.76 |
| 168 | HIS | CG | 7.82 | 47.52 | −0.49 | 12.74 |
| 168 | HIS | CD2 | 7.74 | 46.35 | −1.16 | 12.76 |
| 168 | HIS | ND1 | 7.05 | 47.37 | 0.65 | 12.76 |
| 168 | HIS | CE1 | 6.53 | 46.16 | 0.67 | 12.73 |
| 168 | HIS | NE2 | 6.93 | 45.52 | −0.42 | 12.73 |
| 168 | HIS | C | 9.78 | 48.98 | 1.38 | 12.79 |
| 168 | HIS | O | 9.79 | 48.03 | 2.16 | 12.76 |
| 169 | GLY | N | 9.56 | 50.23 | 1.76 | 12.83 |
| 169 | GLY | CA | 9.29 | 50.51 | 3.15 | 12.88 |
| 169 | GLY | C | 7.92 | 49.94 | 3.40 | 12.99 |
| 169 | GLY | O | 7.12 | 49.80 | 2.47 | 12.89 |
| 170 | VAL | N | 7.63 | 49.59 | 4.65 | 13.09 |
| 170 | VAL | CA | 6.34 | 49.01 | 5.00 | 13.26 |
| 170 | VAL | CB | 6.38 | 47.46 | 4.78 | 13.30 |
| 170 | VAL | CG1 | 7.36 | 46.83 | 5.74 | 13.34 |
| 170 | VAL | CG2 | 4.99 | 46.86 | 4.93 | 13.40 |
| 170 | VAL | C | 6.02 | 49.35 | 6.45 | 13.38 |
| 170 | VAL | O | 6.91 | 49.70 | 7.23 | 13.41 |
| 171 | GLY | N | 4.74 | 49.30 | 6.83 | 13.51 |
| 171 | GLY | CA | 4.39 | 49.61 | 8.20 | 13.74 |
| 171 | GLY | C | 2.95 | 50.07 | 8.37 | 13.93 |
| 171 | GLY | O | 2.14 | 49.36 | 8.96 | 13.79 |
| 172 | LEU | N | 2.64 | 51.26 | 7.88 | 14.20 |
| 172 | LEU | CA | 1.29 | 51.78 | 7.98 | 14.54 |
| 172 | LEU | CB | 1.31 | 53.26 | 8.36 | 14.60 |
| 172 | LEU | CG | 1.73 | 53.49 | 9.81 | 14.67 |
| 172 | LEU | CD1 | 1.92 | 54.98 | 10.08 | 14.80 |
| 172 | LEU | CD2 | 0.67 | 52.91 | 10.74 | 14.68 |
| 172 | LEU | C | 0.59 | 51.57 | 6.64 | 14.81 |
| 172 | LEU | O | −0.56 | 51.95 | 6.45 | 14.71 |
| 173 | SER | N | 1.32 | 50.95 | 5.73 | 15.15 |
| 173 | SER | CA | 0.81 | 50.64 | 4.40 | 15.55 |
| 173 | SER | CB | 0.78 | 51.89 | 3.52 | 15.53 |
| 173 | SER | OG | 2.09 | 52.28 | 3.15 | 15.47 |
| 173 | SER | C | 1.77 | 49.62 | 3.81 | 15.91 |
| 173 | SER | O | 2.86 | 49.38 | 4.33 | 15.91 |
| 174 | LEU | N | 1.35 | 49.02 | 2.71 | 16.34 |
| 174 | LEU | CA | 2.17 | 48.03 | 2.06 | 16.77 |
| 174 | LEU | CB | 1.40 | 47.40 | 0.92 | 16.91 |
| 174 | LEU | CG | 2.30 | 46.51 | 0.10 | 17.13 |
| 174 | LEU | CD1 | 2.45 | 45.16 | 0.79 | 17.23 |
| 174 | LEU | CD2 | 1.71 | 46.41 | −1.30 | 17.19 |
| 174 | LEU | C | 3.44 | 48.68 | 1.50 | 16.89 |
| 174 | LEU | O | 4.54 | 48.16 | 1.69 | 17.05 |
| 175 | HIS | N | 3.25 | 49.80 | 0.82 | 17.03 |
| 175 | HIS | CA | 4.35 | 50.54 | 0.20 | 17.11 |
| 175 | HIS | CB | 4.18 | 50.55 | −1.32 | 17.48 |
| 175 | HIS | CG | 4.58 | 49.26 | −1.98 | 17.81 |
| 175 | HIS | CD2 | 5.52 | 49.00 | −2.92 | 17.96 |
| 175 | HIS | ND1 | 3.98 | 48.06 | −1.70 | 17.98 |
| 175 | HIS | CE1 | 4.53 | 47.10 | −2.43 | 18.00 |
| 175 | HIS | NE2 | 5.47 | 47.65 | −3.18 | 18.08 |
| 175 | HIS | C | 4.51 | 51.99 | 0.68 | 16.94 |
| 175 | HIS | O | 3.62 | 52.82 | 0.47 | 17.03 |
| 176 | GLU | N | 5.64 | 52.27 | 1.32 | 16.72 |
| 176 | GLU | CA | 5.96 | 53.61 | 1.80 | 16.44 |
| 176 | GLU | CB | 5.40 | 53.83 | 3.21 | 16.38 |
| 176 | GLU | CG | 5.67 | 52.73 | 4.22 | 16.29 |
| 176 | GLU | CD | 4.74 | 52.82 | 5.42 | 16.30 |
| 176 | GLU | OE1 | 5.19 | 52.57 | 6.55 | 16.26 |
| 176 | GLU | OE2 | 3.54 | 53.12 | 5.22 | 16.29 |
| 176 | GLU | C | 7.49 | 53.80 | 1.76 | 16.34 |
| 176 | GLU | O | 8.21 | 52.87 | 1.43 | 16.26 |
| 177 | ALA | N | 7.97 | 55.00 | 2.07 | 16.21 |
| 177 | ALA | CA | 9.41 | 55.27 | 2.01 | 16.18 |
| 177 | ALA | CB | 9.68 | 56.71 | 2.42 | 16.15 |
| 177 | ALA | C | 10.24 | 54.32 | 2.87 | 16.14 |
| 177 | ALA | O | 9.87 | 54.00 | 4.00 | 16.17 |
| 178 | PRO | N | 11.38 | 53.84 | 2.33 | 16.11 |
| 178 | PRO | CD | 12.31 | 53.00 | 3.11 | 16.17 |
| 178 | PRO | CA | 11.93 | 54.12 | 1.00 | 16.09 |
| 178 | PRO | CB | 13.36 | 53.61 | 1.11 | 16.14 |
| 178 | PRO | CG | 13.23 | 52.46 | 2.04 | 16.16 |
| 178 | PRO | C | 11.13 | 53.43 | −0.10 | 16.08 |
| 178 | PRO | O | 10.83 | 52.24 | −0.01 | 16.01 |
| 179 | ALA | N | 10.82 | 54.19 | −1.14 | 16.08 |
| 179 | ALA | CA | 10.04 | 53.68 | −2.27 | 16.05 |
| 179 | ALA | CB | 9.97 | 54.74 | −3.36 | 16.06 |
| 179 | ALA | C | 10.55 | 52.38 | −2.85 | 16.00 |
| 179 | ALA | O | 9.78 | 51.45 | −3.09 | 16.04 |
| 180 | HIS | N | 11.86 | 52.30 | −3.08 | 15.96 |
| 180 | HIS | CA | 12.44 | 51.10 | −3.67 | 15.97 |
| 180 | HIS | CB | 12.69 | 51.33 | −5.16 | 16.11 |
| 180 | HIS | CG | 11.46 | 51.65 | −5.95 | 16.34 |
| 180 | HIS | CD2 | 10.52 | 50.84 | −6.49 | 16.39 |
| 180 | HIS | ND1 | 11.06 | 52.94 | −6.23 | 16.46 |
| 180 | HIS | CE1 | 9.93 | 52.91 | −6.91 | 16.48 |
| 180 | HIS | NE2 | 9.58 | 51.65 | −7.08 | 16.54 |
| 180 | HIS | C | 13.72 | 50.61 | −3.04 | 15.84 |
| 180 | HIS | O | 14.66 | 51.37 | −2.81 | 15.88 |
| 181 | VAL | N | 13.74 | 49.31 | −2.76 | 15.72 |
| 181 | VAL | CA | 14.91 | 48.65 | −2.20 | 15.63 |
| 181 | VAL | CB | 14.54 | 47.77 | −0.99 | 15.64 |
| 181 | VAL | CG1 | 15.76 | 47.01 | −0.51 | 15.65 |
| 181 | VAL | CG2 | 13.96 | 48.65 | 0.12 | 15.67 |
| 181 | VAL | C | 15.38 | 47.78 | −3.36 | 15.60 |
| 181 | VAL | O | 14.86 | 46.69 | −3.59 | 15.51 |
| 182 | LEU | N | 16.36 | 48.29 | −4.11 | 15.61 |
| 182 | LEU | CA | 16.88 | 47.59 | −5.28 | 15.63 |
| 182 | LEU | CB | 17.80 | 48.51 | −6.08 | 15.62 |
| 182 | LEU | CG | 17.21 | 49.83 | −6.58 | 15.59 |
| 182 | LEU | CD1 | 18.31 | 50.62 | −7.28 | 15.61 |
| 182 | LEU | CD2 | 16.05 | 49.58 | −7.51 | 15.64 |
| 182 | LEU | C | 17.63 | 46.31 | −4.98 | 15.72 |
| 182 | LEU | O | 18.15 | 46.11 | −3.88 | 15.65 |
| 183 | ASN | N | 17.70 | 45.45 | −5.99 | 15.83 |
| 183 | ASN | CA | 18.42 | 44.19 | −5.89 | 16.02 |
| 183 | ASN | CB | 17.66 | 43.09 | −6.62 | 15.84 |
| 183 | ASN | CG | 16.39 | 42.69 | −5.90 | 15.78 |
| 183 | ASN | OD1 | 16.41 | 42.45 | −4.70 | 15.67 |
| 183 | ASN | ND2 | 15.28 | 42.62 | −6.62 | 15.66 |
| 183 | ASN | C | 19.82 | 44.33 | −6.47 | 16.28 |
| 183 | ASN | O | 20.50 | 43.34 | −6.70 | 16.22 |
| 184 | TYR | N | 20.22 | 45.57 | −6.72 | 16.64 |
| 184 | TYR | CA | 21.55 | 45.84 | −7.26 | 17.08 |
| 184 | TYR | CB | 21.54 | 45.83 | −8.79 | 17.02 |
| 184 | TYR | CG | 20.61 | 46.85 | −9.41 | 17.03 |
| 184 | TYR | CD1 | 19.25 | 46.61 | −9.50 | 17.03 |
| 184 | TYR | CE1 | 18.38 | 47.56 | −10.03 | 17.05 |
| 184 | TYR | CD2 | 21.10 | 48.07 | −9.87 | 17.04 |
| 184 | TYR | CE2 | 20.25 | 49.03 | −10.39 | 17.08 |
| 184 | TYR | CZ | 18.89 | 48.77 | −10.47 | 17.09 |
| 184 | TYR | OH | 18.04 | 49.73 | −10.97 | 17.18 |
| 184 | TYR | C | 22.02 | 47.20 | −6.75 | 17.46 |
| 184 | TYR | O | 21.25 | 47.95 | −6.14 | 17.41 |
| 185 | PHE | N | 23.29 | 47.51 | −7.00 | 17.98 |
| 185 | PHE | CA | 23.89 | 48.74 | −6.53 | 18.57 |
| 185 | PHE | CB | 25.38 | 48.50 | −6.25 | 18.65 |
| 185 | PHE | CG | 26.13 | 49.74 | −5.85 | 18.85 |
| 185 | PHE | CD1 | 25.81 | 50.42 | −4.68 | 18.86 |
| 185 | PHE | CD2 | 27.14 | 50.23 | −6.66 | 18.90 |
| 185 | PHE | CE1 | 26.50 | 51.58 | −4.32 | 18.96 |
| 185 | PHE | CE2 | 27.84 | 51.39 | −6.31 | 18.99 |
| 185 | PHE | CZ | 27.51 | 52.06 | −5.14 | 19.00 |
| 185 | PHE | C | 23.75 | 49.96 | −7.44 | 18.93 |
| 185 | PHE | O | 24.11 | 49.92 | −8.61 | 19.00 |
| 186 | ASP | N | 23.21 | 51.04 | −6.87 | 19.37 |
| 186 | ASP | CA | 23.02 | 52.31 | −7.57 | 19.83 |
| 186 | ASP | CB | 21.56 | 52.75 | −7.48 | 20.05 |
| 186 | ASP | CG | 21.32 | 54.12 | −8.09 | 20.24 |
| 186 | ASP | OD1 | 21.76 | 54.35 | −9.24 | 20.42 |
| 186 | ASP | OD2 | 20.69 | 54.97 | −7.42 | 20.37 |
| 186 | ASP | C | 23.92 | 53.30 | −6.84 | 20.04 |
| 186 | ASP | O | 23.59 | 53.79 | −5.76 | 20.07 |
| 187 | PRO | N | 25.09 | 53.61 | −7.42 | 20.26 |
| 187 | PRO | CD | 25.49 | 53.22 | −8.78 | 20.31 |
| 187 | PRO | CA | 26.07 | 54.54 | −6.85 | 20.42 |
| 187 | PRO | CB | 27.20 | 54.52 | −7.88 | 20.41 |
| 187 | PRO | CG | 26.47 | 54.31 | −9.16 | 20.39 |
| 187 | PRO | C | 25.61 | 55.95 | −6.53 | 20.55 |
| 187 | PRO | O | 26.26 | 56.65 | −5.75 | 20.68 |
| 188 | LYS | N | 24.51 | 56.40 | −7.13 | 20.68 |
| 188 | LYS | CA | 24.04 | 57.75 | −6.87 | 20.77 |
| 188 | LYS | CB | 23.39 | 58.33 | −8.14 | 20.95 |
| 188 | LYS | CG | 22.13 | 57.62 | −8.62 | 21.18 |
| 188 | LYS | CD | 20.93 | 57.96 | −7.76 | 21.39 |
| 188 | LYS | CE | 19.63 | 57.46 | −8.38 | 21.48 |
| 188 | LYS | NZ | 18.47 | 57.71 | −7.48 | 21.69 |
| 188 | LYS | C | 23.08 | 57.89 | −5.69 | 20.73 |
| 188 | LYS | O | 22.75 | 59.00 | −5.29 | 20.77 |
| 189 | ASP | N | 22.65 | 56.77 | −5.12 | 20.62 |
| 189 | ASP | CA | 21.72 | 56.82 | −4.00 | 20.45 |
| 189 | ASP | CB | 20.95 | 55.51 | −3.88 | 20.52 |
| 189 | ASP | CG | 19.82 | 55.58 | −2.86 | 20.63 |
| 189 | ASP | OD1 | 19.43 | 56.70 | −2.49 | 20.65 |
| 189 | ASP | OD2 | 19.32 | 54.51 | −2.45 | 20.64 |
| 189 | ASP | C | 22.43 | 57.13 | −2.69 | 20.29 |
| 189 | ASP | O | 23.32 | 56.40 | −2.27 | 20.29 |
| 190 | LYS | N | 22.02 | 58.22 | −2.05 | 20.07 |
| 190 | LYS | CA | 22.61 | 58.63 | −0.78 | 19.81 |
| 190 | LYS | CB | 22.92 | 60.13 | −0.81 | 20.02 |
| 190 | LYS | CG | 24.00 | 60.53 | −1.79 | 20.26 |
| 190 | LYS | CD | 24.14 | 62.04 | −1.87 | 20.45 |
| 190 | LYS | CE | 25.28 | 62.44 | −2.78 | 20.59 |
| 190 | LYS | NZ | 25.39 | 63.92 | −2.92 | 20.71 |
| 190 | LYS | C | 21.67 | 58.33 | 0.38 | 19.48 |
| 190 | LYS | O | 21.96 | 58.67 | 1.53 | 19.52 |
| 191 | THR | N | 20.53 | 57.71 | 0.07 | 19.05 |
| 191 | THR | CA | 19.53 | 57.37 | 1.08 | 18.60 |
| 191 | THR | CB | 18.33 | 56.62 | 0.46 | 18.67 |
| 191 | THR | OG1 | 17.72 | 57.46 | −0.53 | 18.84 |
| 191 | THR | CG2 | 17.30 | 56.29 | 1.53 | 18.74 |
| 191 | THR | C | 20.12 | 56.49 | 2.17 | 18.18 |
| 191 | THR | O | 20.81 | 55.50 | 1.88 | 18.05 |
| 192 | LEU | N | 19.85 | 56.84 | 3.42 | 17.72 |
| 192 | LEU | CA | 20.37 | 56.06 | 4.55 | 17.28 |
| 192 | LEU | CB | 21.17 | 56.96 | 5.49 | 17.26 |
| 192 | LEU | CG | 22.36 | 57.75 | 4.97 | 17.26 |
| 192 | LEU | CD1 | 22.89 | 58.60 | 6.11 | 17.25 |
| 192 | LEU | CD2 | 23.44 | 56.81 | 4.45 | 17.29 |
| 192 | LEU | C | 19.25 | 55.42 | 5.35 | 17.02 |
| 192 | LEU | O | 18.11 | 55.88 | 5.34 | 16.98 |
| 193 | LEU | N | 19.60 | 54.34 | 6.05 | 16.75 |
| 193 | LEU | CA | 18.64 | 53.65 | 6.90 | 16.48 |
| 193 | LEU | CB | 19.05 | 52.19 | 7.09 | 16.50 |
| 193 | LEU | CG | 19.21 | 51.36 | 5.81 | 16.48 |
| 193 | LEU | CD1 | 19.55 | 49.93 | 6.19 | 16.50 |
| 193 | LEU | CD2 | 17.94 | 51.40 | 4.99 | 16.53 |
| 193 | LEU | C | 18.72 | 54.39 | 8.24 | 16.39 |
| 193 | LEU | O | 19.79 | 54.89 | 8.61 | 16.29 |
| 194 | THR | N | 17.60 | 54.47 | 8.95 | 16.20 |
| 194 | THR | CA | 17.59 | 55.15 | 10.23 | 16.13 |
| 194 | THR | CB | 16.78 | 56.47 | 10.15 | 16.19 |
| 194 | THR | OG1 | 15.40 | 56.17 | 9.89 | 16.28 |
| 194 | THR | CG2 | 17.31 | 57.36 | 9.04 | 16.20 |
| 194 | THR | C | 17.00 | 54.26 | 11.31 | 16.00 |
| 194 | THR | O | 16.30 | 53.29 | 11.03 | 16.03 |
| 195 | GLU | N | 17.30 | 54.59 | 12.56 | 15.89 |
| 195 | GLU | CA | 16.79 | 53.84 | 13.70 | 15.75 |
| 195 | GLU | CB | 17.24 | 54.51 | 14.99 | 15.91 |
| 195 | GLU | CG | 16.64 | 53.94 | 16.26 | 16.23 |
| 195 | GLU | CD | 17.11 | 52.54 | 16.57 | 16.35 |
| 195 | GLU | OE1 | 18.33 | 52.30 | 16.56 | 16.53 |
| 195 | GLU | OE2 | 16.25 | 51.67 | 16.83 | 16.55 |
| 195 | GLU | C | 15.26 | 53.77 | 13.65 | 15.50 |
| 195 | GLU | O | 14.59 | 54.80 | 13.60 | 15.53 |
| 196 | GLY | N | 14.72 | 52.55 | 13.65 | 15.25 |
| 196 | GLY | CA | 13.28 | 52.37 | 13.63 | 14.82 |
| 196 | GLY | C | 12.66 | 52.20 | 12.25 | 14.52 |
| 196 | GLY | O | 11.47 | 51.93 | 12.13 | 14.53 |
| 197 | MET | N | 13.47 | 52.35 | 11.21 | 14.19 |
| 197 | MET | CA | 12.97 | 52.21 | 9.85 | 13.86 |
| 197 | MET | CB | 14.06 | 52.61 | 8.84 | 13.93 |
| 197 | MET | CG | 13.62 | 52.54 | 7.39 | 13.98 |
| 197 | MET | SD | 14.90 | 53.13 | 6.27 | 14.10 |
| 197 | MET | CE | 14.70 | 54.89 | 6.42 | 14.09 |
| 197 | MET | C | 12.52 | 50.77 | 9.58 | 13.58 |
| 197 | MET | O | 13.22 | 49.82 | 9.91 | 13.58 |
| 198 | VAL | N | 11.34 | 50.63 | 8.98 | 13.28 |
| 198 | VAL | CA | 10.80 | 49.31 | 8.66 | 13.03 |
| 198 | VAL | CB | 9.36 | 49.16 | 9.18 | 13.03 |
| 198 | VAL | CG1 | 8.90 | 47.72 | 9.02 | 13.02 |
| 198 | VAL | CG2 | 9.28 | 49.58 | 10.64 | 12.97 |
| 198 | VAL | C | 10.84 | 49.09 | 7.15 | 12.91 |
| 198 | VAL | O | 10.28 | 49.88 | 6.38 | 12.89 |
| 199 | LEU | N | 11.49 | 48.00 | 6.75 | 12.68 |
| 199 | LEU | CA | 11.65 | 47.68 | 5.34 | 12.51 |
| 199 | LEU | CB | 13.13 | 47.81 | 4.94 | 12.76 |
| 199 | LEU | CG | 13.91 | 49.09 | 5.19 | 12.92 |
| 199 | LEU | CD1 | 15.37 | 48.87 | 4.86 | 13.03 |
| 199 | LEU | CD2 | 13.34 | 50.20 | 4.33 | 13.01 |
| 199 | LEU | C | 11.26 | 46.26 | 4.99 | 12.34 |
| 199 | LEU | O | 11.44 | 45.34 | 5.79 | 12.29 |
| 200 | ALA | N | 10.72 | 46.08 | 3.79 | 12.03 |
| 200 | ALA | CA | 10.42 | 44.75 | 3.29 | 11.86 |
| 200 | ALA | CB | 9.11 | 44.74 | 2.51 | 11.79 |
| 200 | ALA | C | 11.61 | 44.52 | 2.35 | 11.70 |
| 200 | ALA | O | 11.76 | 45.22 | 1.34 | 11.61 |
| 201 | ILE | N | 12.48 | 43.59 | 2.72 | 11.57 |
| 201 | ILE | CA | 13.65 | 43.27 | 1.93 | 11.53 |
| 201 | ILE | CB | 14.90 | 43.16 | 2.83 | 11.42 |
| 201 | ILE | CG2 | 16.10 | 42.68 | 2.03 | 11.40 |
| 201 | ILE | CG1 | 15.18 | 44.53 | 3.45 | 11.39 |
| 201 | ILE | CD1 | 16.30 | 44.55 | 4.47 | 11.42 |
| 201 | ILE | C | 13.32 | 41.95 | 1.24 | 11.54 |
| 201 | ILE | O | 13.04 | 40.95 | 1.90 | 11.45 |
| 202 | GLU | N | 13.32 | 41.97 | −0.09 | 11.67 |
| 202 | GLU | CA | 12.93 | 40.80 | −0.86 | 11.84 |
| 202 | GLU | CB | 11.46 | 40.93 | −1.26 | 12.25 |
| 202 | GLU | CG | 11.07 | 42.33 | −1.67 | 12.76 |
| 202 | GLU | CD | 9.64 | 42.43 | −2.17 | 13.10 |
| 202 | GLU | OE1 | 9.07 | 43.53 | −2.10 | 13.26 |
| 202 | GLU | OE2 | 9.09 | 41.42 | −2.64 | 13.49 |
| 202 | GLU | C | 13.75 | 40.49 | −2.10 | 11.74 |
| 202 | GLU | O | 13.29 | 40.68 | −3.23 | 11.72 |
| 203 | PRO | N | 14.96 | 39.98 | −1.91 | 11.62 |
| 203 | PRO | CD | 15.62 | 39.64 | −0.64 | 11.63 |
| 203 | PRO | CA | 15.81 | 39.65 | −3.06 | 11.55 |
| 203 | PRO | CB | 17.08 | 39.12 | −2.40 | 11.58 |
| 203 | PRO | CG | 16.59 | 38.59 | −1.08 | 11.61 |
| 203 | PRO | C | 15.19 | 38.61 | −4.00 | 11.53 |
| 203 | PRO | O | 14.55 | 37.66 | −3.55 | 11.34 |
| 204 | PHE | N | 15.38 | 38.83 | −5.29 | 11.55 |
| 204 | PHE | CA | 14.92 | 37.91 | −6.33 | 11.65 |
| 204 | PHE | CB | 14.06 | 38.60 | −7.39 | 11.88 |
| 204 | PHE | CG | 12.64 | 38.85 | −6.98 | 12.13 |
| 204 | PHE | CD1 | 12.28 | 40.03 | −6.35 | 12.29 |
| 204 | PHE | CD2 | 11.65 | 37.92 | −7.30 | 12.27 |
| 204 | PHE | CE1 | 10.95 | 40.30 | −6.04 | 12.49 |
| 204 | PHE | CE2 | 10.31 | 38.18 | −6.99 | 12.40 |
| 204 | PHE | CZ | 9.96 | 39.37 | −6.36 | 12.45 |
| 204 | PHE | C | 16.18 | 37.41 | −7.04 | 11.61 |
| 204 | PHE | O | 16.94 | 38.22 | −7.58 | 11.69 |
| 205 | ILE | N | 16.39 | 36.10 | −7.03 | 11.58 |
| 205 | ILE | CA | 17.55 | 35.49 | −7.67 | 11.56 |
| 205 | ILE | CB | 18.29 | 34.57 | −6.70 | 11.54 |
| 205 | ILE | CG2 | 19.44 | 33.88 | −7.41 | 11.49 |
| 205 | ILE | CG1 | 18.81 | 35.39 | −5.51 | 11.60 |
| 205 | ILE | CD1 | 19.79 | 36.48 | −5.89 | 11.64 |
| 205 | ILE | C | 17.05 | 34.70 | −8.88 | 11.60 |
| 205 | ILE | O | 16.19 | 33.83 | −8.74 | 11.55 |
| 206 | SER | N | 17.61 | 35.00 | −10.05 | 11.69 |
| 206 | SER | CA | 17.21 | 34.38 | −11.31 | 11.82 |
| 206 | SER | CB | 16.74 | 35.49 | −12.26 | 11.75 |
| 206 | SER | OG | 16.54 | 35.01 | −13.58 | 11.77 |
| 206 | SER | C | 18.26 | 33.53 | −12.01 | 12.02 |
| 206 | SER | O | 19.45 | 33.86 | −11.99 | 11.93 |
| 207 | SER | N | 17.82 | 32.45 | −12.65 | 12.27 |
| 207 | SER | CA | 18.73 | 31.58 | −13.37 | 12.62 |
| 207 | SER | CB | 18.05 | 30.24 | −13.70 | 12.63 |
| 207 | SER | OG | 16.95 | 30.42 | −14.57 | 12.72 |
| 207 | SER | C | 19.27 | 32.22 | −14.65 | 12.88 |
| 207 | SER | O | 20.28 | 31.77 | −15.20 | 12.90 |
| 208 | ASN | N | 18.61 | 33.26 | −15.14 | 13.16 |
| 208 | ASN | CA | 19.06 | 33.94 | −16.35 | 13.48 |
| 208 | ASN | CB | 18.75 | 33.09 | −17.59 | 13.79 |
| 208 | ASN | CG | 19.21 | 33.76 | −18.88 | 14.04 |
| 208 | ASN | OD1 | 18.40 | 34.13 | −19.72 | 14.35 |
| 208 | ASN | ND2 | 20.52 | 33.93 | −19.02 | 14.21 |
| 208 | ASN | C | 18.50 | 35.34 | −16.58 | 13.53 |
| 208 | ASN | O | 19.26 | 36.31 | −16.66 | 13.61 |
| 209 | ALA | N | 17.18 | 35.46 | −16.69 | 13.56 |
| 209 | ALA | CA | 16.54 | 36.76 | −16.93 | 13.59 |
| 209 | ALA | CB | 15.04 | 36.58 | −17.07 | 13.63 |
| 209 | ALA | C | 16.85 | 37.76 | −15.83 | 13.62 |
| 209 | ALA | O | 16.88 | 37.42 | −14.65 | 13.56 |
| 210 | SER | N | 17.07 | 39.01 | −16.23 | 13.67 |
| 210 | SER | CA | 17.38 | 40.07 | −15.29 | 13.77 |
| 210 | SER | CB | 18.59 | 40.88 | −15.77 | 13.87 |
| 210 | SER | OG | 18.33 | 41.50 | −17.02 | 14.18 |
| 210 | SER | C | 16.19 | 41.00 | −15.09 | 13.75 |
| 210 | SER | O | 16.32 | 42.06 | −14.49 | 13.70 |
| 211 | PHE | N | 15.04 | 40.60 | −15.62 | 13.77 |
| 211 | PHE | CA | 13.82 | 41.39 | −15.50 | 13.88 |
| 211 | PHE | CB | 13.84 | 42.56 | −16.49 | 13.84 |
| 211 | PHE | CG | 13.85 | 42.13 | −17.94 | 13.85 |
| 211 | PHE | CD1 | 15.04 | 41.77 | −18.56 | 13.92 |
| 211 | PHE | CD2 | 12.67 | 42.08 | −18.67 | 13.91 |
| 211 | PHE | CE1 | 15.05 | 41.36 | −19.90 | 13.98 |
| 211 | PHE | CE2 | 12.67 | 41.68 | −20.00 | 13.97 |
| 211 | PHE | CZ | 13.87 | 41.32 | −20.62 | 13.99 |
| 211 | PHE | C | 12.59 | 40.53 | −15.75 | 14.04 |
| 211 | PHE | O | 12.70 | 39.42 | −16.27 | 13.89 |
| 212 | VAL | N | 11.42 | 41.04 | −15.37 | 14.30 |
| 212 | VAL | CA | 10.16 | 40.35 | −15.58 | 14.69 |
| 212 | VAL | CB | 9.39 | 40.10 | −14.26 | 14.70 |
| 212 | VAL | CG1 | 10.09 | 39.02 | −13.45 | 14.73 |
| 212 | VAL | CG2 | 9.28 | 41.39 | −13.46 | 14.75 |
| 212 | VAL | C | 9.27 | 41.20 | −16.49 | 15.00 |
| 212 | VAL | O | 9.49 | 42.40 | −16.65 | 15.00 |
| 213 | THR | N | 8.26 | 40.56 | −17.08 | 15.40 |
| 213 | THR | CA | 7.32 | 41.25 | −17.95 | 15.81 |
| 213 | THR | CB | 7.59 | 40.93 | −19.44 | 15.82 |
| 213 | THR | OG1 | 7.63 | 39.51 | −19.63 | 15.83 |
| 213 | THR | CG2 | 8.90 | 41.56 | −19.89 | 15.90 |
| 213 | THR | C | 5.90 | 40.80 | −17.61 | 16.16 |
| 213 | THR | O | 5.71 | 39.86 | −16.85 | 16.09 |
| 214 | GLU | N | 4.90 | 41.48 | −18.18 | 16.59 |
| 214 | GLU | CA | 3.50 | 41.14 | −17.93 | 17.08 |
| 214 | GLU | CB | 2.57 | 42.06 | −18.73 | 17.38 |
| 214 | GLU | CG | 1.09 | 41.77 | −18.50 | 17.97 |
| 214 | GLU | CD | 0.18 | 42.67 | −19.31 | 18.22 |
| 214 | GLU | OE1 | 0.28 | 43.91 | −19.17 | 18.57 |
| 214 | GLU | OE2 | −0.63 | 42.15 | −20.11 | 18.57 |
| 214 | GLU | C | 3.26 | 39.69 | −18.33 | 17.19 |
| 214 | GLU | O | 3.65 | 39.26 | −19.42 | 17.28 |
| 215 | GLY | N | 2.60 | 38.95 | −17.45 | 17.39 |
| 215 | GLY | CA | 2.32 | 37.55 | −17.71 | 17.63 |
| 215 | GLY | C | 0.99 | 37.21 | −18.36 | 17.79 |
| 215 | GLY | O | 0.43 | 38.01 | −19.11 | 17.84 |
| 216 | LYS | N | 0.50 | 36.01 | −18.06 | 17.91 |
| 216 | LYS | CA | −0.75 | 35.49 | −18.61 | 18.05 |
| 216 | LYS | CB | −0.95 | 34.04 | −18.15 | 18.04 |
| 216 | LYS | CG | −1.17 | 33.89 | −16.65 | 17.98 |
| 216 | LYS | CD | −1.44 | 32.44 | −16.25 | 18.01 |
| 216 | LYS | CE | −0.26 | 31.52 | −16.57 | 18.00 |
| 216 | LYS | NZ | 1.01 | 31.98 | −15.93 | 17.99 |
| 216 | LYS | C | −2.00 | 36.30 | −18.25 | 18.16 |
| 216 | LYS | O | −3.07 | 36.05 | −18.80 | 18.23 |
| 217 | ASN | N | −1.85 | 37.24 | −17.33 | 18.22 |
| 217 | ASN | CA | −2.97 | 38.09 | −16.94 | 18.32 |
| 217 | ASN | CB | −3.80 | 37.42 | −15.84 | 18.26 |
| 217 | ASN | CG | −2.97 | 37.00 | −14.64 | 18.22 |
| 217 | ASN | OD1 | −2.30 | 37.83 | −14.03 | 18.20 |
| 217 | ASN | ND2 | −3.00 | 35.72 | −14.31 | 18.16 |
| 217 | ASN | C | −2.46 | 39.46 | −16.48 | 18.40 |
| 217 | ASN | O | −1.27 | 39.73 | −16.54 | 18.44 |
| 218 | GLU | N | −3.37 | 40.32 | −16.04 | 18.51 |
| 218 | GLU | CA | −3.00 | 41.67 | −15.62 | 18.54 |
| 218 | GLU | CB | −4.24 | 42.56 | −15.61 | 18.88 |
| 218 | GLU | CG | −5.35 | 42.05 | −14.71 | 19.43 |
| 218 | GLU | CD | −6.64 | 42.80 | −14.90 | 19.72 |
| 218 | GLU | OE1 | −7.17 | 42.78 | −16.04 | 20.03 |
| 218 | GLU | OE2 | −7.14 | 43.40 | −13.93 | 20.04 |
| 218 | GLU | C | −2.30 | 41.76 | −14.28 | 18.33 |
| 218 | GLU | O | −1.99 | 42.86 | −13.82 | 18.37 |
| 219 | TRP | N | −2.03 | 40.62 | −13.65 | 18.06 |
| 219 | TRP | CA | −1.37 | 40.63 | −12.35 | 17.79 |
| 219 | TRP | CB | −2.25 | 39.95 | −11.30 | 17.92 |
| 219 | TRP | CG | −3.48 | 40.73 | −10.95 | 18.07 |
| 219 | TRP | CD2 | −3.54 | 41.88 | −10.11 | 18.14 |
| 219 | TRP | CE2 | −4.90 | 42.28 | −10.04 | 18.17 |
| 219 | TRP | CE3 | −2.59 | 42.63 | −9.41 | 18.17 |
| 219 | TRP | CD1 | −4.76 | 40.47 | −11.34 | 18.12 |
| 219 | TRP | NE1 | −5.62 | 41.40 | −10.80 | 18.15 |
| 219 | TRP | CZ2 | −5.32 | 43.38 | −9.30 | 18.20 |
| 219 | TRP | CZ3 | −3.01 | 43.73 | −8.67 | 18.21 |
| 219 | TRP | CH2 | −4.36 | 44.10 | −8.62 | 18.25 |
| 219 | TRP | C | 0.01 | 39.98 | −12.32 | 17.54 |
| 219 | TRP | O | 1.00 | 40.61 | −11.94 | 17.48 |
| 220 | ALA | N | 0.07 | 38.72 | −12.72 | 17.25 |
| 220 | ALA | CA | 1.31 | 37.95 | −12.69 | 16.94 |
| 220 | ALA | CB | 1.02 | 36.52 | −13.11 | 16.99 |
| 220 | ALA | C | 2.48 | 38.50 | −13.50 | 16.73 |
| 220 | ALA | O | 2.31 | 39.01 | −14.60 | 16.71 |
| 221 | PHE | N | 3.68 | 38.39 | −12.91 | 16.46 |
| 221 | PHE | CA | 4.92 | 38.80 | −13.56 | 16.21 |
| 221 | PHE | CB | 5.82 | 39.59 | −12.60 | 16.43 |
| 221 | PHE | CG | 5.40 | 41.02 | −12.41 | 16.64 |
| 221 | PHE | CD1 | 5.53 | 41.94 | −13.44 | 16.77 |
| 221 | PHE | CD2 | 4.88 | 41.44 | −11.19 | 16.77 |
| 221 | PHE | CE1 | 5.14 | 43.27 | −13.26 | 16.86 |
| 221 | PHE | CE2 | 4.48 | 42.77 | −11.00 | 16.87 |
| 221 | PHE | CZ | 4.62 | 43.68 | −12.04 | 16.88 |
| 221 | PHE | C | 5.62 | 37.50 | −13.95 | 15.93 |
| 221 | PHE | O | 5.68 | 36.56 | −13.15 | 15.85 |
| 222 | GLU | N | 6.13 | 37.44 | −15.17 | 15.59 |
| 222 | GLU | CA | 6.81 | 36.23 | −15.64 | 15.32 |
| 222 | GLU | CB | 5.86 | 35.44 | −16.56 | 15.34 |
| 222 | GLU | CG | 4.56 | 34.98 | −15.89 | 15.50 |
| 222 | GLU | CD | 3.58 | 34.32 | −16.85 | 15.61 |
| 222 | GLU | OE1 | 4.04 | 33.76 | −17.87 | 15.75 |
| 222 | GLU | OE2 | 2.36 | 34.36 | −16.58 | 15.60 |
| 222 | GLU | C | 8.05 | 36.62 | −16.44 | 15.10 |
| 222 | GLU | O | 8.35 | 37.80 | −16.58 | 15.04 |
| 223 | THR | N | 8.77 | 35.62 | −16.92 | 14.92 |
| 223 | THR | CA | 9.94 | 35.85 | −17.77 | 14.75 |
| 223 | THR | CB | 11.27 | 35.33 | −17.16 | 14.67 |
| 223 | THR | OG1 | 11.20 | 33.91 | −16.98 | 14.50 |
| 223 | THR | CG2 | 11.55 | 36.00 | −15.82 | 14.56 |
| 223 | THR | C | 9.63 | 35.04 | −19.01 | 14.77 |
| 223 | THR | O | 9.18 | 33.89 | −18.92 | 14.75 |
| 224 | SER | N | 9.83 | 35.63 | −20.18 | 14.79 |
| 224 | SER | CA | 9.54 | 34.93 | −21.42 | 14.79 |
| 224 | SER | CB | 9.66 | 35.88 | −22.62 | 14.80 |
| 224 | SER | OG | 8.56 | 36.78 | −22.64 | 14.94 |
| 224 | SER | C | 10.43 | 33.71 | −21.63 | 14.72 |
| 224 | SER | O | 9.99 | 32.73 | −22.24 | 14.72 |
| 225 | ASP | N | 11.66 | 33.75 | −21.12 | 14.67 |
| 225 | ASP | CA | 12.57 | 32.61 | −21.27 | 14.62 |
| 225 | ASP | CB | 14.04 | 33.07 | −21.25 | 14.77 |
| 225 | ASP | CG | 14.44 | 33.75 | −19.95 | 14.93 |
| 225 | ASP | OD1 | 13.78 | 33.52 | −18.92 | 14.86 |
| 225 | ASP | OD2 | 15.45 | 34.50 | −19.96 | 15.13 |
| 225 | ASP | C | 12.34 | 31.56 | −20.18 | 14.54 |
| 225 | ASP | O | 13.07 | 30.57 | −20.10 | 14.58 |
| 226 | LYS | N | 11.32 | 31.78 | −19.36 | 14.43 |
| 226 | LYS | CA | 10.95 | 30.88 | −18.28 | 14.28 |
| 226 | LYS | CB | 10.33 | 29.60 | −18.86 | 14.53 |
| 226 | LYS | CG | 9.05 | 29.87 | −19.63 | 14.91 |
| 226 | LYS | CD | 8.36 | 28.57 | −20.06 | 15.34 |
| 226 | LYS | CE | 7.06 | 28.86 | −20.78 | 15.60 |
| 226 | LYS | NZ | 7.32 | 29.65 | −22.01 | 15.93 |
| 226 | LYS | C | 12.09 | 30.54 | −17.33 | 14.02 |
| 226 | LYS | O | 12.31 | 29.38 | −16.97 | 13.99 |
| 227 | SER | N | 12.82 | 31.58 | −16.93 | 13.72 |
| 227 | SER | CA | 13.92 | 31.42 | −15.99 | 13.45 |
| 227 | SER | CB | 14.62 | 32.75 | −15.75 | 13.59 |
| 227 | SER | OG | 15.35 | 33.19 | −16.88 | 13.71 |
| 227 | SER | C | 13.35 | 30.94 | −14.67 | 13.24 |
| 227 | SER | O | 12.17 | 31.13 | −14.38 | 13.17 |
| 228 | PHE | N | 14.21 | 30.33 | −13.86 | 12.95 |
| 228 | PHE | CA | 13.79 | 29.89 | −12.54 | 12.69 |
| 228 | PHE | CB | 14.46 | 28.58 | −12.16 | 12.83 |
| 228 | PHE | CG | 14.13 | 27.46 | −13.09 | 12.93 |
| 228 | PHE | CD1 | 12.82 | 27.27 | −13.53 | 13.03 |
| 228 | PHE | CD2 | 15.11 | 26.59 | −13.54 | 13.06 |
| 228 | PHE | CE1 | 12.49 | 26.23 | −14.39 | 13.02 |
| 228 | PHE | CE2 | 14.80 | 25.55 | −14.40 | 13.09 |
| 228 | PHE | CZ | 13.48 | 25.37 | −14.84 | 13.05 |
| 228 | PHE | C | 14.20 | 31.03 | −11.62 | 12.44 |
| 228 | PHE | O | 15.37 | 31.42 | −11.56 | 12.33 |
| 229 | VAL | N | 13.22 | 31.58 | −10.92 | 12.20 |
| 229 | VAL | CA | 13.47 | 32.71 | −10.05 | 12.03 |
| 229 | VAL | CB | 12.77 | 33.97 | −10.59 | 12.00 |
| 229 | VAL | CG1 | 13.14 | 35.18 | −9.75 | 12.02 |
| 229 | VAL | CG2 | 13.16 | 34.18 | −12.05 | 12.03 |
| 229 | VAL | C | 12.98 | 32.43 | −8.64 | 11.94 |
| 229 | VAL | O | 11.86 | 31.95 | −8.44 | 11.99 |
| 230 | ALA | N | 13.83 | 32.72 | −7.66 | 11.78 |
| 230 | ALA | CA | 13.47 | 32.50 | −6.27 | 11.78 |
| 230 | ALA | CB | 14.41 | 31.47 | −5.64 | 11.74 |
| 230 | ALA | C | 13.53 | 33.80 | −5.48 | 11.78 |
| 230 | ALA | O | 14.36 | 34.68 | −5.74 | 11.68 |
| 231 | GLN | N | 12.64 | 33.91 | −4.51 | 11.78 |
| 231 | GLN | CA | 12.59 | 35.08 | −3.67 | 11.88 |
| 231 | GLN | CB | 11.39 | 35.94 | −4.05 | 12.21 |
| 231 | GLN | CG | 11.13 | 37.07 | −3.10 | 12.71 |
| 231 | GLN | CD | 10.00 | 37.95 | −3.56 | 12.96 |
| 231 | GLN | OE1 | 8.94 | 37.46 | −3.96 | 13.24 |
| 231 | GLN | NE2 | 10.21 | 39.26 | −3.50 | 13.22 |
| 231 | GLN | C | 12.48 | 34.69 | −2.20 | 11.78 |
| 231 | GLN | O | 11.93 | 33.65 | −1.86 | 11.81 |
| 232 | ILE | N | 13.06 | 35.53 | −1.34 | 11.66 |
| 232 | ILE | CA | 12.97 | 35.36 | 0.11 | 11.66 |
| 232 | ILE | CB | 14.30 | 34.91 | 0.75 | 11.80 |
| 232 | ILE | CG2 | 14.22 | 35.07 | 2.27 | 11.90 |
| 232 | ILE | CG1 | 14.60 | 33.46 | 0.38 | 11.98 |
| 232 | ILE | CD1 | 13.56 | 32.47 | 0.88 | 12.15 |
| 232 | ILE | C | 12.64 | 36.77 | 0.56 | 11.58 |
| 232 | ILE | O | 13.34 | 37.71 | 0.20 | 11.65 |
| 233 | GLU | N | 11.58 | 36.91 | 1.35 | 11.43 |
| 233 | GLU | CA | 11.16 | 38.22 | 1.80 | 11.27 |
| 233 | GLU | CB | 9.94 | 38.67 | 0.99 | 11.59 |
| 233 | GLU | CG | 9.28 | 39.94 | 1.49 | 12.15 |
| 233 | GLU | CD | 8.13 | 40.38 | 0.60 | 12.42 |
| 233 | GLU | OE1 | 7.29 | 39.53 | 0.23 | 12.46 |
| 233 | GLU | OE2 | 8.08 | 41.58 | 0.27 | 12.86 |
| 233 | GLU | C | 10.83 | 38.27 | 3.28 | 11.00 |
| 233 | GLU | O | 10.21 | 37.36 | 3.82 | 10.79 |
| 234 | HIS | N | 11.27 | 39.34 | 3.93 | 10.79 |
| 234 | HIS | CA | 11.02 | 39.56 | 5.34 | 10.60 |
| 234 | HIS | CB | 12.19 | 39.10 | 6.20 | 10.47 |
| 234 | HIS | CG | 12.21 | 37.63 | 6.47 | 10.27 |
| 234 | HIS | CD2 | 13.09 | 36.67 | 6.09 | 10.23 |
| 234 | HIS | ND1 | 11.25 | 37.00 | 7.22 | 10.25 |
| 234 | HIS | CE1 | 11.52 | 35.71 | 7.30 | 10.24 |
| 234 | HIS | NE2 | 12.64 | 35.48 | 6.62 | 10.27 |
| 234 | HIS | C | 10.80 | 41.03 | 5.63 | 10.60 |
| 234 | HIS | O | 11.30 | 41.91 | 4.92 | 10.46 |
| 235 | THR | N | 10.03 | 41.29 | 6.68 | 10.71 |
| 235 | THR | CA | 9.80 | 42.66 | 7.12 | 10.90 |
| 235 | THR | CB | 8.38 | 42.86 | 7.67 | 10.73 |
| 235 | THR | OG1 | 7.45 | 42.74 | 6.59 | 10.47 |
| 235 | THR | CG2 | 8.24 | 44.25 | 8.29 | 10.59 |
| 235 | THR | C | 10.85 | 42.84 | 8.22 | 11.17 |
| 235 | THR | O | 10.90 | 42.07 | 9.18 | 11.15 |
| 236 | VAL | N | 11.68 | 43.86 | 8.04 | 11.56 |
| 236 | VAL | CA | 12.79 | 44.13 | 8.93 | 12.10 |
| 236 | VAL | CB | 14.12 | 44.02 | 8.14 | 12.05 |
| 236 | VAL | CG1 | 15.32 | 44.20 | 9.06 | 12.16 |
| 236 | VAL | CG2 | 14.18 | 42.68 | 7.40 | 12.05 |
| 236 | VAL | C | 12.74 | 45.52 | 9.56 | 12.41 |
| 236 | VAL | O | 12.44 | 46.50 | 8.89 | 12.45 |
| 237 | ILE | N | 13.06 | 45.59 | 10.85 | 12.88 |
| 237 | ILE | CA | 13.10 | 46.87 | 11.55 | 13.46 |
| 237 | ILE | CB | 12.38 | 46.80 | 12.90 | 13.41 |
| 237 | ILE | CG2 | 12.40 | 48.18 | 13.56 | 13.48 |
| 237 | ILE | CG1 | 10.93 | 46.34 | 12.70 | 13.51 |
| 237 | ILE | CD1 | 10.12 | 46.29 | 13.99 | 13.61 |
| 237 | ILE | C | 14.57 | 47.13 | 11.82 | 13.83 |
| 237 | ILE | O | 15.26 | 46.27 | 12.38 | 13.85 |
| 238 | VAL | N | 15.05 | 48.30 | 11.41 | 14.39 |
| 238 | VAL | CA | 16.44 | 48.68 | 11.61 | 15.04 |
| 238 | VAL | CB | 16.86 | 49.74 | 10.57 | 14.98 |
| 238 | VAL | CG1 | 18.32 | 50.12 | 10.77 | 15.06 |
| 238 | VAL | CG2 | 16.62 | 49.21 | 9.16 | 15.10 |
| 238 | VAL | C | 16.61 | 49.26 | 13.02 | 15.42 |
| 238 | VAL | O | 15.94 | 50.23 | 13.38 | 15.55 |
| 239 | THR | N | 17.47 | 48.65 | 13.83 | 15.99 |
| 239 | THR | CA | 17.72 | 49.14 | 15.19 | 16.57 |
| 239 | THR | CB | 17.02 | 48.27 | 16.26 | 16.57 |
| 239 | THR | OG1 | 17.81 | 47.11 | 16.53 | 16.68 |
| 239 | THR | CG2 | 15.62 | 47.85 | 15.80 | 16.66 |
| 239 | THR | C | 19.21 | 49.13 | 15.48 | 16.93 |
| 239 | THR | O | 19.98 | 48.42 | 14.82 | 16.98 |
| 240 | LYS | N | 19.63 | 49.91 | 16.47 | 17.40 |
| 240 | LYS | CA | 21.04 | 50.00 | 16.83 | 17.87 |
| 240 | LYS | CB | 21.24 | 51.07 | 17.91 | 18.07 |
| 240 | LYS | CG | 20.39 | 50.88 | 19.14 | 18.31 |
| 240 | LYS | CD | 20.61 | 52.00 | 20.15 | 18.57 |
| 240 | LYS | CE | 19.68 | 51.88 | 21.34 | 18.74 |
| 240 | LYS | NZ | 18.25 | 51.89 | 20.92 | 18.95 |
| 240 | LYS | C | 21.62 | 48.68 | 17.30 | 18.09 |
| 240 | LYS | O | 22.83 | 48.46 | 17.20 | 18.22 |
| 241 | ASP | N | 20.77 | 47.80 | 17.80 | 18.33 |
| 241 | ASP | CA | 21.22 | 46.50 | 18.29 | 18.48 |
| 241 | ASP | CB | 20.50 | 46.14 | 19.59 | 18.81 |
| 241 | ASP | CG | 20.80 | 47.12 | 20.70 | 19.03 |
| 241 | ASP | OD1 | 22.00 | 47.31 | 21.02 | 19.26 |
| 241 | ASP | OD2 | 19.84 | 47.70 | 21.25 | 19.26 |
| 241 | ASP | C | 21.01 | 45.39 | 17.27 | 18.42 |
| 241 | ASP | O | 21.00 | 44.21 | 17.64 | 18.49 |
| 242 | GLY | N | 20.84 | 45.76 | 16.01 | 18.26 |
| 242 | GLY | CA | 20.65 | 44.75 | 14.98 | 18.01 |
| 242 | GLY | C | 19.26 | 44.75 | 14.39 | 17.79 |
| 242 | GLY | O | 18.32 | 45.29 | 14.98 | 17.91 |
| 243 | PRO | N | 19.09 | 44.16 | 13.20 | 17.56 |
| 243 | PRO | CD | 20.11 | 43.57 | 12.31 | 17.53 |
| 243 | PRO | CA | 17.77 | 44.12 | 12.57 | 17.29 |
| 243 | PRO | CB | 18.08 | 43.72 | 11.13 | 17.35 |
| 243 | PRO | CG | 19.27 | 42.83 | 11.30 | 17.44 |
| 243 | PRO | C | 16.80 | 43.15 | 13.24 | 17.01 |
| 243 | PRO | O | 17.18 | 42.05 | 13.63 | 17.04 |
| 244 | ILE | N | 15.55 | 43.58 | 13.36 | 16.67 |
| 244 | ILE | CA | 14.50 | 42.76 | 13.95 | 16.32 |
| 244 | ILE | CB | 13.61 | 43.56 | 14.92 | 16.40 |
| 244 | ILE | CG2 | 12.45 | 42.69 | 15.41 | 16.43 |
| 244 | ILE | CG1 | 14.43 | 44.07 | 16.10 | 16.49 |
| 244 | ILE | CD1 | 13.62 | 44.89 | 17.10 | 16.63 |
| 244 | ILE | C | 13.60 | 42.30 | 12.81 | 15.98 |
| 244 | ILE | O | 13.01 | 43.12 | 12.12 | 15.97 |
| 245 | LEU | N | 13.51 | 40.99 | 12.60 | 15.63 |
| 245 | LEU | CA | 12.65 | 40.45 | 11.55 | 15.26 |
| 245 | LEU | CB | 13.27 | 39.20 | 10.92 | 15.29 |
| 245 | LEU | CG | 14.36 | 39.36 | 9.87 | 15.41 |
| 245 | LEU | CD1 | 15.57 | 40.07 | 10.46 | 15.45 |
| 245 | LEU | CD2 | 14.74 | 37.98 | 9.35 | 15.44 |
| 245 | LEU | C | 11.31 | 40.09 | 12.16 | 15.01 |
| 245 | LEU | O | 11.20 | 39.09 | 12.87 | 15.02 |
| 246 | THR | N | 10.29 | 40.90 | 11.90 | 14.65 |
| 246 | THR | CA | 8.97 | 40.65 | 12.46 | 14.33 |
| 246 | THR | CB | 8.02 | 41.83 | 12.23 | 14.19 |
| 246 | THR | OG1 | 7.74 | 41.95 | 10.83 | 13.98 |
| 246 | THR | CG2 | 8.64 | 43.13 | 12.73 | 14.07 |
| 246 | THR | C | 8.33 | 39.41 | 11.84 | 14.27 |
| 246 | THR | O | 7.44 | 38.81 | 12.43 | 14.09 |
| 247 | THR | N | 8.81 | 39.02 | 10.66 | 14.28 |
| 247 | THR | CA | 8.24 | 37.88 | 9.96 | 14.41 |
| 247 | THR | CB | 8.06 | 38.17 | 8.47 | 14.24 |
| 247 | THR | OG1 | 9.28 | 38.68 | 7.94 | 14.02 |
| 247 | THR | CG2 | 6.95 | 39.19 | 8.27 | 14.06 |
| 247 | THR | C | 9.02 | 36.58 | 10.11 | 14.72 |
| 247 | THR | O | 8.84 | 35.63 | 9.35 | 14.58 |
| 248 | LYS | N | 9.91 | 36.54 | 11.10 | 15.14 |
| 248 | LYS | CA | 10.68 | 35.34 | 11.35 | 15.67 |
| 248 | LYS | CB | 12.18 | 35.62 | 11.38 | 15.82 |
| 248 | LYS | CG | 13.01 | 34.36 | 11.62 | 16.14 |
| 248 | LYS | CD | 14.49 | 34.66 | 11.63 | 16.43 |
| 248 | LYS | CE | 15.29 | 33.43 | 12.02 | 16.61 |
| 248 | LYS | NZ | 16.75 | 33.73 | 12.07 | 16.87 |
| 248 | LYS | C | 10.26 | 34.77 | 12.70 | 15.94 |
| 248 | LYS | O | 10.18 | 35.49 | 13.70 | 16.02 |
| 249 | ILE | N | 9.95 | 33.48 | 12.70 | 16.30 |
| 249 | ILE | CA | 9.55 | 32.77 | 13.90 | 16.70 |
| 249 | ILE | CB | 8.39 | 31.80 | 13.62 | 16.72 |
| 249 | ILE | CG2 | 8.12 | 30.92 | 14.84 | 16.75 |
| 249 | ILE | CG1 | 7.13 | 32.59 | 13.24 | 16.71 |
| 249 | ILE | CD1 | 5.91 | 31.73 | 12.96 | 16.67 |
| 249 | ILE | C | 10.78 | 31.99 | 14.34 | 16.95 |
| 249 | ILE | O | 10.93 | 30.84 | 13.88 | 17.20 |
| 249 | ILE | OT | 11.59 | 32.55 | 15.11 | 17.26 |
| 301 | HOH | O | 10.50 | 34.31 | 4.32 | 9.06 |
| 302 | HOH | O | 5.57 | 43.83 | 10.68 | 9.81 |
| 303 | HOH | O | −1.47 | 37.94 | 1.86 | 12.48 |
| 304 | HOH | O | 9.79 | 31.23 | −7.03 | 11.64 |
| 305 | HOH | O | 3.89 | 34.95 | −5.27 | 11.03 |
| 306 | HOH | O | 15.78 | 21.59 | −8.09 | 10.36 |
| 307 | HOH | O | 1.48 | 24.02 | −11.09 | 12.98 |
| 308 | HOH | O | 2.97 | 15.64 | −9.85 | 13.15 |
| 309 | HOH | O | 7.92 | 29.43 | 5.32 | 13.72 |
| 310 | HOH | O | 0.01 | 23.07 | 6.64 | 14.54 |
| 311 | HOH | O | 15.63 | 45.72 | −8.03 | 12.44 |
| 312 | HOH | O | 10.09 | 28.99 | −15.19 | 11.95 |
| 313 | HOH | O | 18.01 | 39.43 | −18.99 | 16.32 |
| 314 | HOH | O | −0.95 | 44.41 | 14.47 | 13.37 |
| 315 | HOH | O | −0.66 | 38.44 | 20.34 | 14.96 |
| 316 | HOH | O | −1.55 | 34.10 | −12.27 | 12.53 |
| 317 | HOH | O | 19.17 | 48.37 | −2.30 | 15.29 |
| 318 | HOH | O | 0.12 | 30.41 | −9.59 | 13.28 |
| 319 | HOH | O | 1.38 | 39.43 | −0.52 | 14.20 |
| 320 | HOH | O | 17.49 | 50.72 | −2.91 | 16.84 |
| 321 | HOH | O | 17.02 | 43.84 | −2.49 | 13.68 |
| 322 | HOH | O | 28.15 | 32.65 | −2.10 | 15.80 |
| 323 | HOH | O | 6.84 | 38.82 | 15.12 | 12.81 |
| 324 | HOH | O | 6.59 | 25.34 | 6.77 | 15.48 |
| 325 | HOH | O | 12.11 | 26.54 | 2.56 | 15.55 |
| 326 | HOH | O | 9.17 | 23.20 | 1.33 | 16.34 |
| 327 | HOH | O | 12.02 | 32.56 | 8.27 | 16.56 |
| 328 | HOH | O | −9.74 | 24.52 | 0.67 | 20.03 |
| 329 | HOH | O | 10.45 | 30.81 | −11.10 | 14.20 |
| 330 | HOH | O | 27.52 | 35.27 | −0.58 | 18.28 |
| 331 | HOH | O | 25.11 | 45.75 | −8.22 | 17.53 |
| 332 | HOH | O | −1.30 | 30.42 | −12.80 | 15.06 |
| 333 | HOH | O | 19.02 | 56.83 | 12.96 | 16.84 |
| 334 | HOH | O | −8.50 | 38.80 | 4.37 | 16.78 |
| 335 | HOH | O | −6.38 | 18.76 | −3.02 | 19.40 |
| 336 | HOH | O | −10.59 | 34.20 | −0.11 | 17.10 |
| 337 | HOH | O | 21.30 | 50.53 | −4.76 | 18.10 |
| 338 | HOH | O | 21.45 | 28.88 | 1.32 | 19.23 |
| 339 | HOH | O | 21.88 | 54.48 | −0.26 | 16.58 |
| 340 | HOH | O | 6.49 | 38.98 | −4.48 | 17.69 |
| 341 | HOH | O | 0.11 | 48.00 | 19.88 | 17.22 |
| 342 | HOH | O | 10.10 | 52.62 | 6.29 | 15.23 |
| 343 | HOH | O | 21.00 | 22.73 | −6.38 | 16.72 |
| 344 | HOH | O | 24.86 | 54.24 | −3.40 | 22.28 |
| 345 | HOH | O | 7.92 | 55.12 | 5.89 | 19.85 |
| 346 | HOH | O | 14.71 | 39.07 | 14.56 | 19.07 |
| 347 | HOH | O | 26.86 | 45.72 | 0.92 | 19.88 |
| 348 | HOH | O | 22.02 | 40.61 | −12.44 | 18.41 |
| 349 | HOH | O | 22.74 | 38.00 | 8.33 | 19.02 |
| 350 | HOH | O | −9.45 | 24.82 | −6.24 | 21.83 |
| 351 | HOH | O | 13.78 | 37.27 | −21.84 | 19.18 |
| 352 | HOH | O | 4.99 | 28.20 | 12.08 | 20.77 |
| 353 | HOH | O | −5.01 | 33.74 | −15.69 | 20.64 |
| 354 | HOH | O | 1.17 | 27.89 | −16.92 | 20.59 |
| 355 | HOH | O | 0.17 | 44.15 | 19.11 | 19.87 |
| 356 | HOH | O | −3.41 | 22.84 | −13.56 | 21.42 |
| 357 | HOH | O | −5.14 | 28.62 | −13.47 | 20.90 |
| 358 | HOH | O | −1.85 | 19.72 | 1.80 | 20.71 |
| 359 | HOH | O | 6.48 | 21.09 | −1.06 | 20.45 |
| 360 | HOH | O | 28.11 | 42.45 | −8.13 | 18.62 |
| 361 | HOH | O | −12.26 | 26.83 | −2.91 | 22.09 |
| 362 | HOH | O | 6.67 | 56.62 | 9.97 | 19.68 |
| 363 | HOH | O | 4.01 | 47.86 | 26.45 | 17.27 |
| 364 | HOH | O | 18.69 | 59.47 | 3.77 | 23.06 |
| 365 | HOH | O | 0.16 | 20.37 | 6.01 | 23.36 |
| 366 | HOH | O | 22.30 | 49.21 | 4.75 | 18.77 |
| 367 | HOH | O | −7.86 | 33.69 | −6.02 | 19.95 |
| 368 | HOH | O | 10.46 | 12.87 | −7.36 | 18.01 |
| 369 | HOH | O | 11.00 | 20.66 | −13.11 | 21.77 |
| 370 | HOH | O | 14.21 | 19.07 | −4.63 | 19.48 |
| 371 | HOH | O | 27.85 | 28.82 | −6.89 | 22.74 |
| 372 | HOH | O | −6.38 | 47.34 | 10.32 | 18.97 |
| 373 | HOH | O | −10.01 | 39.83 | 10.28 | 20.93 |
| 374 | HOH | O | 26.70 | 64.11 | 0.15 | 20.24 |
| 375 | HOH | O | 3.20 | 24.97 | 9.63 | 25.45 |
| 376 | HOH | O | −2.60 | 51.70 | 4.38 | 20.78 |
| 377 | HOH | O | 13.88 | 54.45 | −2.97 | 20.82 |
| 378 | HOH | O | 6.18 | 26.81 | −16.96 | 18.19 |
| 379 | HOH | O | 10.58 | 28.33 | 8.39 | 23.90 |
| 380 | HOH | O | 7.39 | 21.08 | −12.28 | 23.32 |
| 381 | HOH | O | 0.13 | 47.78 | 24.35 | 22.62 |
| 382 | HOH | O | 26.15 | 36.21 | −12.96 | 20.40 |
| 383 | HOH | O | 8.96 | 24.34 | −14.66 | 17.57 |
| 384 | HOH | O | −1.01 | 26.64 | −15.30 | 20.12 |
| 385 | HOH | O | 11.88 | 19.84 | −2.20 | 20.50 |
| 386 | HOH | O | 24.49 | 44.87 | 4.87 | 22.28 |
| 387 | HOH | O | −10.21 | 35.98 | 5.99 | 18.66 |
| 388 | HOH | O | 9.21 | 13.44 | −4.01 | 25.85 |
| 389 | HOH | O | 14.98 | 29.54 | 2.33 | 24.61 |
| 390 | HOH | O | 7.93 | 59.43 | 12.87 | 24.17 |
| 391 | HOH | O | −0.26 | 23.55 | 11.19 | 23.78 |
| 392 | HOH | O | −12.06 | 39.39 | 7.08 | 24.15 |
| 393 | HOH | O | 4.98 | 40.38 | −21.60 | 22.64 |
| 394 | HOH | O | 10.69 | 29.72 | 6.01 | 18.43 |
| 395 | HOH | O | 24.36 | 29.29 | −12.38 | 22.73 |
| 396 | HOH | O | 28.22 | 43.44 | −0.25 | 25.25 |
| 397 | HOH | O | −6.56 | 51.42 | 11.24 | 21.57 |
| 398 | HOH | O | −13.34 | 29.31 | 1.75 | 23.21 |
| 399 | HOH | O | 19.63 | 47.05 | 12.32 | 20.88 |
| 400 | HOH | O | 4.95 | 19.45 | 5.14 | 23.22 |
| 401 | HOH | O | 20.89 | 22.41 | −14.55 | 24.54 |
| 402 | HOH | O | 23.32 | 50.74 | −11.13 | 22.14 |
| 403 | HOH | O | 24.62 | 27.54 | −2.49 | 22.10 |
| 404 | HOH | O | 10.41 | 25.28 | −17.89 | 22.27 |
| 405 | HOH | O | 12.30 | 57.28 | 5.52 | 24.16 |
| 406 | HOH | O | 4.64 | 24.76 | −15.47 | 24.23 |
| 407 | HOH | O | 27.73 | 38.82 | 4.59 | 24.95 |
| 408 | HOH | O | 17.31 | 29.62 | −17.22 | 21.80 |
| 409 | HOH | O | 11.27 | 56.97 | −1.16 | 23.68 |
| 410 | HOH | O | 26.93 | 42.10 | 3.67 | 21.90 |
| 411 | HOH | O | 8.72 | 44.98 | −17.85 | 21.75 |
| 412 | HOH | O | 8.03 | 47.25 | −4.59 | 21.41 |
| 413 | HOH | O | 4.14 | 21.80 | −13.55 | 24.99 |
| 414 | HOH | O | 10.17 | 30.45 | −23.51 | 22.48 |
| 415 | HOH | O | 2.62 | 20.87 | 9.35 | 24.58 |
| 416 | HOH | O | −3.74 | 18.10 | −1.15 | 25.06 |
| 417 | HOH | O | 2.04 | 18.34 | 2.49 | 25.70 |
| 418 | HOH | O | −7.43 | 45.03 | 12.12 | 22.88 |
| 419 | HOH | O | 3.58 | 30.30 | −20.07 | 26.38 |
| 420 | HOH | O | −10.04 | 21.86 | −8.06 | 26.34 |
| 421 | HOH | O | 6.53 | 32.82 | −19.37 | 22.03 |
| 422 | HOH | O | 26.25 | 50.66 | −1.04 | 22.34 |
| 423 | HOH | O | 4.96 | 12.79 | −7.27 | 21.96 |
| 424 | HOH | O | 30.77 | 47.25 | 7.48 | 24.89 |
| 425 | HOH | O | 5.92 | 21.44 | 8.83 | 26.99 |
| 426 | HOH | O | 7.46 | 53.62 | 23.92 | 23.43 |
| 427 | HOH | O | 24.97 | 34.17 | 3.43 | 22.85 |
| 428 | HOH | O | −6.37 | 22.14 | 2.31 | 24.19 |
| 429 | HOH | O | 14.77 | 22.58 | −16.65 | 23.42 |
| 430 | HOH | O | 3.57 | 54.77 | −1.91 | 25.37 |
| 431 | HOH | O | 22.09 | 24.38 | −11.40 | 21.89 |
| 432 | HOH | O | −3.17 | 29.64 | −17.16 | 24.28 |
| 433 | HOH | O | 13.39 | 57.89 | 1.13 | 23.28 |
| 434 | HOH | O | 12.52 | 49.15 | 17.84 | 24.31 |
| 435 | HOH | O | −2.08 | 47.31 | 18.20 | 24.25 |
| 436 | HOH | O | −8.48 | 32.84 | −9.78 | 23.74 |
| 437 | HOH | O | 7.89 | 56.57 | −0.91 | 25.49 |
| 438 | HOH | O | 2.24 | 54.17 | 19.67 | 21.14 |
| 439 | HOH | O | 20.06 | 27.95 | 5.10 | 26.39 |
| 440 | HOH | O | 27.74 | 54.69 | −2.52 | 22.59 |
| 441 | HOH | O | 5.78 | 17.42 | −1.67 | 24.72 |
| 442 | HOH | O | −2.46 | 21.51 | 9.49 | 26.75 |
| 443 | HOH | O | 14.66 | 33.57 | 6.02 | 25.13 |
| 444 | HOH | O | 9.66 | 29.46 | 10.67 | 17.72 |
| 445 | HOH | O | 24.67 | 52.28 | 13.32 | 23.17 |
| 446 | HOH | O | 26.52 | 38.92 | −13.27 | 24.93 |
| 447 | HOH | O | 25.12 | 34.05 | 7.68 | 26.03 |
| 448 | HOH | O | 26.01 | 25.28 | −8.92 | 24.50 |
| 449 | HOH | O | 25.09 | 25.29 | −5.13 | 25.06 |
| 450 | HOH | O | 8.27 | 17.86 | −15.20 | 26.25 |
| 451 | HOH | O | 22.34 | 30.07 | −15.27 | 24.43 |
| 452 | HOH | O | 18.62 | 34.74 | −23.36 | 24.16 |
| 453 | HOH | O | 10.20 | 50.69 | 19.86 | 25.64 |
| 454 | HOH | O | 4.22 | 37.79 | −5.22 | 11.06 |
| 455 | HOH | O | 2.45 | 13.45 | −8.33 | 15.64 |
| 456 | HOH | O | −2.56 | 29.86 | −9.55 | 15.16 |
| 457 | HOH | O | −9.51 | 38.40 | 6.83 | 15.69 |
| 458 | HOH | O | −3.38 | 31.98 | −11.92 | 14.11 |
| 459 | HOH | O | 19.26 | 52.40 | −4.23 | 17.67 |
| 460 | HOH | O | 13.22 | 27.93 | 0.45 | 17.00 |
| 461 | HOH | O | 8.82 | 26.70 | −16.12 | 17.14 |
| 462 | HOH | O | −9.17 | 31.97 | −0.82 | 16.61 |
| 463 | HOH | O | 14.39 | 43.99 | −1.92 | 14.00 |
| 464 | HOH | O | 26.84 | 44.47 | −6.39 | 16.06 |
| 465 | HOH | O | −1.84 | 44.64 | 17.02 | 19.03 |
| 466 | HOH | O | 10.06 | 34.39 | −7.29 | 15.99 |
| 467 | HOH | O | 1.18 | 23.04 | 9.02 | 18.90 |
| 468 | HOH | O | 12.44 | 38.17 | −18.77 | 15.25 |
| 469 | HOH | O | 7.30 | 23.12 | 5.05 | 19.32 |
| 470 | HOH | O | −11.23 | 38.31 | 12.21 | 20.31 |
| 471 | HOH | O | 28.60 | 30.16 | −0.68 | 18.62 |
| 472 | HOH | O | −0.73 | 26.20 | 11.41 | 21.74 |
| 473 | HOH | O | 24.27 | 39.15 | −11.42 | 19.76 |
| 474 | HOH | O | 4.55 | 14.77 | −12.02 | 19.53 |
| 475 | HOH | O | −0.59 | 18.81 | 4.03 | 21.27 |
| 476 | HOH | O | 5.93 | 24.23 | 9.11 | 22.95 |
| 477 | HOH | O | −8.21 | 22.36 | −10.67 | 21.87 |
| 478 | HOH | O | −3.98 | 21.05 | 3.15 | 22.46 |
| 479 | HOH | O | −8.85 | 28.81 | −12.01 | 22.91 |
| 480 | HOH | O | 5.97 | 44.02 | −19.72 | 23.69 |
| 481 | HOH | O | −10.40 | 40.78 | 3.46 | 22.30 |
| 482 | HOH | O | 4.50 | 19.36 | −14.69 | 22.88 |
| 483 | HOH | O | 14.04 | 31.14 | 4.55 | 21.83 |
| 484 | HOH | O | 30.51 | 41.31 | −7.64 | 23.60 |
| 485 | HOH | O | 21.07 | 36.06 | 8.80 | 23.18 |
| 486 | HOH | O | 4.08 | 29.47 | 16.54 | 23.69 |
| 487 | HOH | O | 29.17 | 37.84 | −0.16 | 21.62 |
| 488 | HOH | O | 20.00 | 54.35 | 17.19 | 23.32 |
| 489 | HOH | O | 15.94 | 30.37 | −19.31 | 24.71 |
| 490 | HOH | O | 20.46 | 60.80 | 2.12 | 23.18 |
| 491 | HOH | O | 23.44 | 22.82 | −8.19 | 24.08 |
| 492 | HOH | O | −4.17 | 51.23 | −1.97 | 24.09 |
| 493 | HOH | O | −11.19 | 28.95 | 3.59 | 22.64 |
| 494 | HOH | O | 6.98 | 22.69 | −15.48 | 23.33 |
| 495 | HOH | O | 8.12 | 30.80 | −9.06 | 13.51 |
| 496 | HOH | O | 13.70 | 31.03 | −1.77 | 16.61 |
| 497 | HOH | O | −13.99 | 28.89 | −2.79 | 22.29 |
| 498 | HOH | O | −5.62 | 32.23 | −13.46 | 19.99 |
| 499 | HOH | O | 8.76 | 17.86 | 8.23 | 23.45 |
| 500 | HOH | O | 19.03 | 37.00 | −21.38 | 22.06 |
| 300 | 737 | C1 | 1.79 | 42.89 | −2.36 | 31.43 |
| 300 | 737 | C2 | 3.20 | 42.72 | −2.05 | 31.13 |
| 300 | 737 | C3 | 1.29 | 42.49 | −3.63 | 31.62 |
| 300 | 737 | C4 | 0.88 | 43.44 | −1.42 | 31.83 |
| 300 | 737 | C5 | 4.24 | 43.62 | −1.92 | 31.00 |
| 300 | 737 | N6 | 3.81 | 41.53 | −1.80 | 30.99 |
| 300 | 737 | C7 | −0.08 | 42.66 | −3.93 | 31.70 |
| 300 | 737 | C8 | −0.49 | 43.61 | −1.73 | 31.96 |
| 300 | 737 | N9 | 5.39 | 42.96 | −1.62 | 30.83 |
| 300 | 737 | N10 | 5.12 | 41.67 | −1.54 | 30.83 |
| 300 | 737 | C11 | −0.97 | 43.22 | −2.99 | 31.79 |
| 300 | 737 | I12 | −0.82 | 42.07 | −5.76 | 31.66 |
| 253 | ZN2 | ZN | 6.76 | 43.46 | −0.66 | 18.13 |
| 254 | ZN2 | ZN | 5.51 | 40.30 | −0.30 | 13.79 |

TABLE III
Provides a three dimensional protein coordinate set of an
S. aureus methionine aminopeptidase crystalline structure
of an inhibitor complex with 5-benzofuran-2-yl-1-
H-[1,2,3]triazole.

| Residue Atom | | | X | Y | Z | B |
|---|---|---|---|---|---|---|
| 1 | MET | CB | 6.23 | 53.31 | 9.47 | 20.78 |
| 1 | MET | CG | 6.51 | 52.11 | 10.37 | 20.85 |
| 1 | MET | SD | 5.13 | 51.60 | 11.41 | 21.11 |
| 1 | MET | CE | 5.27 | 52.76 | 12.79 | 21.00 |
| 1 | MET | C | 8.66 | 53.80 | 9.55 | 20.75 |
| 1 | MET | O | 9.60 | 53.03 | 9.40 | 20.70 |
| 1 | MET | N | 7.67 | 52.75 | 7.54 | 20.67 |
| 1 | MET | CA | 7.44 | 53.73 | 8.64 | 20.72 |
| 2 | ILE | N | 8.62 | 54.73 | 10.50 | 20.85 |
| 2 | ILE | CA | 9.70 | 54.87 | 11.47 | 20.87 |
| 2 | ILE | CB | 10.29 | 56.30 | 11.47 | 20.94 |
| 2 | ILE | CG2 | 11.44 | 56.37 | 12.46 | 21.01 |
| 2 | ILE | CG1 | 10.75 | 56.69 | 10.06 | 21.03 |
| 2 | ILE | CD1 | 11.83 | 55.79 | 9.48 | 21.06 |
| 2 | ILE | C | 9.12 | 54.61 | 12.85 | 20.86 |
| 2 | ILE | O | 8.28 | 55.37 | 13.32 | 20.90 |
| 3 | VAL | N | 9.55 | 53.52 | 13.49 | 20.88 |
| 3 | VAL | CA | 9.07 | 53.16 | 14.82 | 20.92 |
| 3 | VAL | CB | 9.53 | 51.74 | 15.22 | 20.97 |
| 3 | VAL | CG1 | 9.06 | 51.41 | 16.63 | 20.98 |
| 3 | VAL | CG2 | 8.96 | 50.72 | 14.23 | 21.00 |
| 3 | VAL | C | 9.60 | 54.13 | 15.87 | 21.00 |
| 3 | VAL | O | 10.78 | 54.47 | 15.88 | 20.98 |
| 4 | LYS | N | 8.71 | 54.57 | 16.76 | 21.11 |
| 4 | LYS | CA | 9.07 | 55.49 | 17.83 | 21.18 |
| 4 | LYS | CB | 8.49 | 56.88 | 17.57 | 21.34 |
| 4 | LYS | CG | 9.12 | 57.66 | 16.42 | 21.64 |
| 4 | LYS | CD | 10.60 | 57.93 | 16.66 | 21.83 |
| 4 | LYS | CE | 11.19 | 58.79 | 15.55 | 22.01 |
| 4 | LYS | NZ | 12.66 | 58.96 | 15.69 | 22.18 |
| 4 | LYS | C | 8.60 | 55.01 | 19.20 | 21.13 |
| 4 | LYS | O | 9.21 | 55.33 | 20.22 | 21.15 |
| 5 | THR | N | 7.50 | 54.25 | 19.24 | 21.00 |
| 5 | THR | CA | 6.97 | 53.76 | 20.52 | 20.92 |
| 5 | THR | CB | 5.52 | 54.26 | 20.76 | 20.92 |
| 5 | THR | OG1 | 4.63 | 53.63 | 19.83 | 20.79 |
| 5 | THR | CG2 | 5.44 | 55.77 | 20.61 | 20.97 |
| 5 | THR | C | 6.95 | 52.24 | 20.65 | 20.90 |
| 5 | THR | O | 6.92 | 51.51 | 19.65 | 20.87 |
| 6 | GLU | N | 6.94 | 51.77 | 21.89 | 20.82 |
| 6 | GLU | CA | 6.90 | 50.35 | 22.19 | 20.66 |
| 6 | GLU | CB | 6.96 | 50.14 | 23.71 | 21.01 |
| 6 | GLU | CG | 6.69 | 48.72 | 24.21 | 21.47 |
| 6 | GLU | CD | 7.79 | 47.74 | 23.85 | 21.71 |
| 6 | GLU | OE1 | 8.97 | 48.04 | 24.12 | 22.01 |
| 6 | GLU | OE2 | 7.47 | 46.65 | 23.30 | 21.88 |
| 6 | GLU | C | 5.61 | 49.75 | 21.62 | 20.36 |
| 6 | GLU | O | 5.59 | 48.61 | 21.17 | 20.34 |
| 7 | GLU | N | 4.53 | 50.54 | 21.62 | 19.97 |
| 7 | GLU | CA | 3.25 | 50.07 | 21.11 | 19.65 |
| 7 | GLU | CB | 2.14 | 51.09 | 21.39 | 19.99 |
| 7 | GLU | CG | 1.90 | 51.35 | 22.87 | 20.37 |
| 7 | GLU | CD | 3.12 | 51.88 | 23.58 | 20.60 |
| 7 | GLU | OE1 | 3.70 | 52.89 | 23.12 | 20.78 |
| 7 | GLU | OE2 | 3.52 | 51.29 | 24.60 | 20.94 |
| 7 | GLU | C | 3.31 | 49.78 | 19.61 | 19.22 |
| 7 | GLU | O | 2.76 | 48.77 | 19.14 | 19.19 |
| 8 | GLU | N | 3.96 | 50.66 | 18.85 | 18.76 |
| 8 | GLU | CA | 4.07 | 50.45 | 17.41 | 18.24 |
| 8 | GLU | CB | 4.80 | 51.62 | 16.75 | 18.37 |
| 8 | GLU | CG | 4.10 | 52.95 | 16.93 | 18.47 |
| 8 | GLU | CD | 4.88 | 54.13 | 16.34 | 18.61 |
| 8 | GLU | OE1 | 6.12 | 54.15 | 16.47 | 18.62 |
| 8 | GLU | OE2 | 4.24 | 55.04 | 15.77 | 18.60 |
| 8 | GLU | C | 4.84 | 49.16 | 17.16 | 17.83 |
| 8 | GLU | O | 4.47 | 48.36 | 16.30 | 17.74 |
| 9 | LEU | N | 5.92 | 48.97 | 17.92 | 17.39 |
| 9 | LEU | CA | 6.74 | 47.77 | 17.78 | 16.96 |
| 9 | LEU | CB | 7.94 | 47.83 | 18.73 | 17.07 |
| 9 | LEU | CG | 8.84 | 46.59 | 18.79 | 17.08 |
| 9 | LEU | CD1 | 9.61 | 46.42 | 17.49 | 17.25 |
| 9 | LEU | CD2 | 9.82 | 46.74 | 19.95 | 17.22 |
| 9 | LEU | C | 5.93 | 46.51 | 18.05 | 16.71 |
| 9 | LEU | O | 6.06 | 45.52 | 17.34 | 16.62 |
| 10 | GLN | N | 5.10 | 46.55 | 19.09 | 16.29 |
| 10 | GLN | CA | 4.29 | 45.38 | 19.42 | 15.87 |
| 10 | GLN | CB | 3.57 | 45.61 | 20.75 | 15.94 |
| 10 | GLN | CG | 4.47 | 46.02 | 21.89 | 16.18 |
| 10 | GLN | CD | 3.70 | 46.16 | 23.18 | 16.27 |
| 10 | GLN | OE1 | 2.49 | 46.40 | 23.16 | 16.32 |
| 10 | GLN | NE2 | 4.39 | 46.03 | 24.31 | 16.41 |
| 10 | GLN | C | 3.27 | 45.10 | 18.32 | 15.52 |
| 10 | GLN | O | 3.00 | 43.95 | 17.99 | 15.45 |
| 11 | ALA | N | 2.71 | 46.16 | 17.75 | 15.03 |
| 11 | ALA | CA | 1.73 | 45.98 | 16.69 | 14.60 |
| 11 | ALA | CB | 1.09 | 47.31 | 16.35 | 14.54 |
| 11 | ALA | C | 2.39 | 45.37 | 15.45 | 14.31 |
| 11 | ALA | O | 1.81 | 44.50 | 14.81 | 14.30 |
| 12 | LEU | N | 3.60 | 45.81 | 15.13 | 13.92 |
| 12 | LEU | CA | 4.33 | 45.31 | 13.96 | 13.64 |
| 12 | LEU | CB | 5.56 | 46.19 | 13.69 | 13.67 |
| 12 | LEU | CG | 5.21 | 47.59 | 13.15 | 13.74 |
| 12 | LEU | CD1 | 6.39 | 48.55 | 13.25 | 13.76 |
| 12 | LEU | CD2 | 4.77 | 47.43 | 11.71 | 13.80 |
| 12 | LEU | C | 4.76 | 43.86 | 14.18 | 13.43 |
| 12 | LEU | O | 4.74 | 43.05 | 13.25 | 13.36 |
| 13 | LYS | N | 5.14 | 43.52 | 15.41 | 13.19 |
| 13 | LYS | CA | 5.54 | 42.16 | 15.72 | 13.00 |
| 13 | LYS | CB | 6.16 | 42.09 | 17.10 | 13.18 |
| 13 | LYS | CG | 7.50 | 42.76 | 17.19 | 13.41 |
| 13 | LYS | CD | 8.02 | 42.67 | 18.60 | 13.64 |
| 13 | LYS | CE | 9.47 | 43.09 | 18.69 | 13.93 |
| 13 | LYS | NZ | 9.92 | 43.01 | 20.10 | 14.02 |
| 13 | LYS | C | 4.32 | 41.24 | 15.64 | 12.76 |
| 13 | LYS | O | 4.43 | 40.09 | 15.21 | 12.72 |
| 14 | GLU | N | 3.17 | 41.76 | 16.05 | 12.46 |
| 14 | GLU | CA | 1.93 | 40.99 | 16.02 | 12.11 |
| 14 | GLU | CB | 0.77 | 41.80 | 16.62 | 12.31 |
| 14 | GLU | CG | −0.62 | 41.17 | 16.42 | 12.52 |
| 14 | GLU | CD | −0.81 | 39.88 | 17.21 | 12.69 |
| 14 | GLU | OE1 | 0.13 | 39.43 | 17.88 | 12.87 |
| 14 | GLU | OE2 | −1.93 | 39.32 | 17.15 | 12.91 |
| 14 | GLU | C | 1.58 | 40.58 | 14.59 | 11.74 |
| 14 | GLU | O | 1.42 | 39.39 | 14.30 | 11.61 |
| 15 | ILE | N | 1.46 | 41.56 | 13.69 | 11.42 |
| 15 | ILE | CA | 1.12 | 41.28 | 12.29 | 11.11 |
| 15 | ILE | CB | 0.78 | 42.60 | 11.50 | 11.27 |
| 15 | ILE | CG2 | 2.03 | 43.48 | 11.35 | 11.24 |
| 15 | ILE | CG1 | 0.24 | 42.25 | 10.11 | 11.31 |
| 15 | ILE | CD1 | −0.99 | 41.37 | 10.13 | 11.43 |
| 15 | ILE | C | 2.25 | 40.52 | 11.60 | 10.90 |
| 15 | ILE | O | 2.02 | 39.76 | 10.65 | 10.72 |
| 16 | GLY | N | 3.48 | 40.72 | 12.09 | 10.67 |
| 16 | GLY | CA | 4.62 | 40.03 | 11.52 | 10.38 |
| 16 | GLY | C | 4.49 | 38.55 | 11.79 | 10.35 |
| 16 | GLY | O | 4.72 | 37.72 | 10.91 | 10.24 |
| 17 | TYR | N | 4.11 | 38.21 | 13.02 | 10.35 |
| 17 | TYR | CA | 3.94 | 36.81 | 13.42 | 10.40 |
| 17 | TYR | CB | 3.61 | 36.70 | 14.91 | 10.66 |
| 17 | TYR | CG | 3.27 | 35.28 | 15.31 | 11.00 |
| 17 | TYR | CD1 | 4.27 | 34.31 | 15.39 | 11.22 |
| 17 | TYR | CE1 | 3.95 | 32.97 | 15.62 | 11.39 |
| 17 | TYR | CD2 | 1.94 | 34.88 | 15.49 | 11.15 |
| 17 | TYR | CE2 | 1.61 | 33.53 | 15.72 | 11.36 |
| 17 | TYR | CZ | 2.62 | 32.59 | 15.78 | 11.45 |
| 17 | TYR | OH | 2.32 | 31.26 | 15.97 | 11.77 |
| 17 | TYR | C | 2.84 | 36.12 | 12.63 | 10.23 |
| 17 | TYR | O | 2.99 | 34.96 | 12.22 | 10.16 |
| 18 | ILE | N | 1.73 | 36.83 | 12.46 | 10.03 |
| 18 | ILE | CA | 0.59 | 36.30 | 11.73 | 9.79 |
| 18 | ILE | CB | −0.58 | 37.31 | 11.72 | 9.69 |
| 18 | ILE | CG2 | −1.73 | 36.80 | 10.87 | 9.62 |
| 18 | ILE | CG1 | −1.09 | 37.53 | 13.15 | 9.55 |
| 18 | ILE | CD1 | −2.16 | 38.61 | 13.25 | 9.44 |
| 18 | ILE | C | 0.97 | 35.95 | 10.29 | 9.75 |
| 18 | ILE | O | 0.61 | 34.87 | 9.80 | 9.71 |
| 19 | CYS | N | 1.70 | 36.84 | 9.62 | 9.73 |
| 19 | CYS | CA | 2.11 | 36.60 | 8.24 | 9.68 |
| 19 | CYS | CB | 2.73 | 37.86 | 7.65 | 9.70 |
| 19 | CYS | SG | 1.44 | 39.11 | 7.32 | 10.06 |
| 19 | CYS | C | 3.07 | 35.42 | 8.14 | 9.66 |
| 19 | CYS | O | 2.97 | 34.60 | 7.22 | 9.70 |
| 20 | ALA | N | 3.99 | 35.34 | 9.09 | 9.68 |
| 20 | ALA | CA | 4.95 | 34.25 | 9.11 | 9.59 |
| 20 | ALA | CB | 5.97 | 34.48 | 10.22 | 9.56 |
| 20 | ALA | C | 4.20 | 32.95 | 9.35 | 9.67 |
| 20 | ALA | O | 4.44 | 31.95 | 8.67 | 9.54 |
| 21 | LYS | N | 3.30 | 32.95 | 10.33 | 9.79 |
| 21 | LYS | CA | 2.50 | 31.78 | 10.66 | 10.05 |
| 21 | LYS | CB | 1.51 | 32.15 | 11.78 | 10.30 |
| 21 | LYS | CG | 0.58 | 31.04 | 12.20 | 10.86 |
| 21 | LYS | CD | 1.31 | 29.84 | 12.79 | 11.34 |
| 21 | LYS | CE | 0.26 | 28.87 | 13.32 | 11.72 |
| 21 | LYS | NZ | 0.83 | 27.67 | 13.98 | 12.13 |
| 21 | LYS | C | 1.75 | 31.27 | 9.43 | 9.97 |
| 21 | LYS | O | 1.74 | 30.06 | 9.12 | 9.98 |
| 22 | VAL | N | 1.12 | 32.20 | 8.72 | 9.93 |
| 22 | VAL | CA | 0.38 | 31.88 | 7.50 | 9.95 |
| 22 | VAL | CB | −0.36 | 33.13 | 6.98 | 10.03 |
| 22 | VAL | CG1 | −0.96 | 32.87 | 5.60 | 10.07 |
| 22 | VAL | CG2 | −1.42 | 33.53 | 7.96 | 10.08 |
| 22 | VAL | C | 1.31 | 31.35 | 6.42 | 9.85 |
| 22 | VAL | O | 1.04 | 30.32 | 5.80 | 9.82 |
| 23 | ARG | N | 2.42 | 32.06 | 6.20 | 9.83 |
| 23 | ARG | CA | 3.39 | 31.65 | 5.19 | 9.75 |
| 23 | ARG | CB | 4.59 | 32.59 | 5.21 | 9.74 |
| 23 | ARG | CG | 5.64 | 32.34 | 4.13 | 9.71 |
| 23 | ARG | CD | 6.95 | 32.96 | 4.58 | 9.70 |
| 23 | ARG | NE | 7.41 | 32.29 | 5.79 | 9.57 |
| 23 | ARG | CZ | 7.94 | 32.88 | 6.86 | 9.58 |
| 23 | ARG | NH1 | 8.11 | 34.20 | 6.88 | 9.29 |
| 23 | ARG | NH2 | 8.27 | 32.15 | 7.91 | 9.55 |
| 23 | ARG | C | 3.86 | 30.23 | 5.48 | 9.81 |
| 23 | ARG | O | 3.94 | 29.39 | 4.57 | 9.75 |
| 24 | ASN | N | 4.18 | 29.96 | 6.74 | 9.88 |
| 24 | ASN | CA | 4.64 | 28.62 | 7.09 | 10.04 |
| 24 | ASN | CB | 5.06 | 28.56 | 8.57 | 9.93 |
| 24 | ASN | CG | 6.37 | 29.29 | 8.85 | 9.88 |
| 24 | ASN | OD1 | 7.20 | 29.50 | 7.95 | 9.56 |
| 24 | ASN | ND2 | 6.58 | 29.65 | 10.11 | 9.88 |
| 24 | ASN | C | 3.59 | 27.55 | 6.81 | 10.17 |
| 24 | ASN | O | 3.89 | 26.51 | 6.21 | 10.24 |
| 25 | THR | N | 2.35 | 27.79 | 7.24 | 10.41 |
| 25 | THR | CA | 1.26 | 26.83 | 7.05 | 10.44 |
| 25 | THR | CB | −0.04 | 27.34 | 7.73 | 10.52 |
| 25 | THR | OG1 | 0.23 | 27.61 | 9.11 | 10.64 |
| 25 | THR | CG2 | −1.14 | 26.28 | 7.67 | 10.62 |
| 25 | THR | C | 1.00 | 26.52 | 5.57 | 10.50 |
| 25 | THR | O | 0.81 | 25.36 | 5.18 | 10.34 |
| 26 | MET | N | 1.00 | 27.56 | 4.74 | 10.47 |
| 26 | MET | CA | 0.79 | 27.37 | 3.31 | 10.49 |
| 26 | MET | CB | 0.64 | 28.73 | 2.61 | 10.26 |
| 26 | MET | CG | −0.64 | 29.45 | 3.00 | 10.07 |
| 26 | MET | SD | −0.73 | 31.14 | 2.39 | 9.79 |
| 26 | MET | CE | −0.94 | 30.86 | 0.63 | 9.76 |
| 26 | MET | C | 1.92 | 26.57 | 2.68 | 10.63 |
| 26 | MET | O | 1.68 | 25.70 | 1.83 | 10.61 |
| 27 | GLN | N | 3.15 | 26.84 | 3.10 | 10.93 |
| 27 | GLN | CA | 4.30 | 26.11 | 2.57 | 11.23 |
| 27 | GLN | CB | 5.62 | 26.70 | 3.10 | 11.29 |
| 27 | GLN | CG | 6.85 | 25.91 | 2.64 | 11.57 |
| 27 | GLN | CD | 8.17 | 26.61 | 2.95 | 11.74 |
| 27 | GLN | OE1 | 8.25 | 27.45 | 3.85 | 11.92 |
| 27 | GLN | NE2 | 9.21 | 26.27 | 2.20 | 11.86 |
| 27 | GLN | C | 4.20 | 24.62 | 2.93 | 11.35 |
| 27 | GLN | O | 4.36 | 23.76 | 2.07 | 11.29 |
| 28 | ALA | N | 3.90 | 24.32 | 4.20 | 11.48 |
| 28 | ALA | CA | 3.79 | 22.93 | 4.62 | 11.66 |
| 28 | ALA | CB | 3.62 | 22.85 | 6.15 | 11.56 |
| 28 | ALA | C | 2.66 | 22.17 | 3.93 | 11.83 |
| 28 | ALA | O | 2.69 | 20.94 | 3.84 | 11.78 |
| 29 | ALA | N | 1.66 | 22.90 | 3.45 | 11.99 |
| 29 | ALA | CA | 0.51 | 22.28 | 2.79 | 12.13 |
| 29 | ALA | CB | −0.76 | 23.07 | 3.09 | 12.00 |
| 29 | ALA | C | 0.70 | 22.19 | 1.28 | 12.31 |
| 29 | ALA | O | −0.13 | 21.61 | 0.57 | 12.29 |
| 30 | THR | N | 1.77 | 22.79 | 0.79 | 12.52 |
| 30 | THR | CA | 2.05 | 22.76 | −0.64 | 12.72 |
| 30 | THR | CB | 3.00 | 23.90 | −1.03 | 12.75 |
| 30 | THR | OG1 | 2.40 | 25.16 | −0.70 | 12.88 |
| 30 | THR | CG2 | 3.27 | 23.87 | −2.52 | 12.81 |
| 30 | THR | C | 2.65 | 21.42 | −1.05 | 12.80 |
| 30 | THR | O | 3.78 | 21.10 | −0.69 | 12.93 |
| 31 | LYS | N | 1.86 | 20.64 | −1.78 | 12.96 |
| 31 | LYS | CA | 2.28 | 19.33 | −2.26 | 13.21 |
| 31 | LYS | CB | 1.95 | 18.25 | −1.23 | 13.42 |
| 31 | LYS | CG | 0.47 | 18.02 | −1.01 | 13.76 |
| 31 | LYS | CD | 0.22 | 16.85 | −0.06 | 14.09 |
| 31 | LYS | CE | −1.17 | 16.30 | −0.25 | 14.29 |
| 31 | LYS | NZ | −1.46 | 15.12 | 0.63 | 14.48 |
| 31 | LYS | C | 1.57 | 19.02 | −3.57 | 13.26 |
| 31 | LYS | O | 0.56 | 19.65 | −3.90 | 13.22 |
| 32 | PRO | N | 2.10 | 18.07 | −4.35 | 13.40 |
| 32 | PRO | CD | 3.33 | 17.28 | −4.14 | 13.45 |
| 32 | PRO | CA | 1.46 | 17.71 | −5.62 | 13.55 |
| 32 | PRO | CB | 2.25 | 16.49 | −6.08 | 13.54 |
| 32 | PRO | CG | 3.62 | 16.77 | −5.53 | 13.44 |
| 32 | PRO | C | −0.01 | 17.38 | −5.34 | 13.68 |
| 32 | PRO | O | −0.32 | 16.74 | −4.33 | 13.73 |
| 33 | GLY | N | −0.92 | 17.84 | −6.20 | 13.78 |
| 33 | GLY | CA | −2.33 | 17.55 | −6.00 | 13.83 |
| 33 | GLY | C | −3.19 | 18.67 | −5.44 | 13.87 |
| 33 | GLY | O | −4.41 | 18.67 | −5.63 | 13.98 |
| 34 | ILE | N | −2.59 | 19.64 | −4.76 | 13.80 |
| 34 | ILE | CA | −3.36 | 20.74 | −4.20 | 13.72 |
| 34 | ILE | CB | −2.76 | 21.18 | −2.84 | 13.65 |
| 34 | ILE | CG2 | −1.49 | 21.98 | −3.06 | 13.55 |
| 34 | ILE | CG1 | −3.76 | 22.06 | −2.09 | 13.66 |
| 34 | ILE | CD1 | −3.31 | 22.35 | −0.67 | 13.71 |
| 34 | ILE | C | −3.39 | 21.93 | −5.16 | 13.68 |
| 34 | ILE | O | −2.43 | 22.15 | −5.90 | 13.69 |
| 35 | THR | N | −4.47 | 22.70 | −5.18 | 13.68 |
| 35 | THR | CA | −4.53 | 23.86 | −6.06 | 13.67 |
| 35 | THR | CB | −5.95 | 24.13 | −6.60 | 13.59 |
| 35 | THR | OG1 | −6.77 | 24.64 | −5.54 | 13.57 |
| 35 | THR | CG2 | −6.56 | 22.86 | −7.17 | 13.64 |
| 35 | THR | C | −4.10 | 25.10 | −5.28 | 13.73 |
| 35 | THR | O | −4.19 | 25.14 | −4.05 | 13.65 |
| 36 | THR | N | −3.64 | 26.12 | −6.00 | 13.87 |
| 36 | THR | CA | −3.21 | 27.36 | −5.39 | 14.04 |
| 36 | THR | CB | −2.60 | 28.34 | −6.44 | 14.10 |
| 36 | THR | OG1 | −3.55 | 28.61 | −7.48 | 14.22 |
| 36 | THR | CG2 | −1.34 | 27.74 | −7.05 | 14.07 |
| 36 | THR | C | −4.38 | 28.01 | −4.67 | 14.06 |
| 36 | THR | O | −4.21 | 28.67 | −3.65 | 14.06 |
| 37 | LYS | N | −5.58 | 27.80 | −5.20 | 14.22 |
| 37 | LYS | CA | −6.77 | 28.35 | −4.56 | 14.33 |
| 37 | LYS | CB | −8.02 | 28.10 | −5.40 | 14.58 |
| 37 | LYS | CG | −9.30 | 28.61 | −4.76 | 14.86 |
| 37 | LYS | CD | −9.25 | 30.12 | −4.54 | 15.26 |
| 37 | LYS | CE | −10.58 | 30.65 | −4.05 | 15.48 |
| 37 | LYS | NZ | −10.49 | 32.09 | −3.71 | 15.79 |
| 37 | LYS | C | −6.94 | 27.70 | −3.18 | 14.31 |
| 37 | LYS | O | −7.29 | 28.37 | −2.22 | 14.34 |
| 38 | GLU | N | −6.70 | 26.39 | −3.10 | 14.28 |
| 38 | GLU | CA | −6.81 | 25.69 | −1.82 | 14.31 |
| 38 | GLU | CB | −6.57 | 24.19 | −1.98 | 14.63 |
| 38 | GLU | CG | −7.73 | 23.43 | −2.62 | 15.20 |
| 38 | GLU | CD | −7.35 | 22.01 | −2.95 | 15.58 |
| 38 | GLU | OE1 | −6.74 | 21.79 | −4.01 | 15.73 |
| 38 | GLU | OE2 | −7.64 | 21.11 | −2.12 | 15.93 |
| 38 | GLU | C | −5.79 | 26.25 | −0.83 | 14.17 |
| 38 | GLU | O | −6.07 | 26.38 | 0.36 | 14.01 |
| 39 | LEU | N | −4.60 | 26.57 | −1.34 | 13.96 |
| 39 | LEU | CA | −3.55 | 27.14 | −0.51 | 13.89 |
| 39 | LEU | CB | −2.24 | 27.23 | −1.29 | 13.76 |
| 39 | LEU | CG | −1.60 | 25.88 | −1.61 | 13.65 |
| 39 | LEU | CD1 | −0.39 | 26.12 | −2.51 | 13.52 |
| 39 | LEU | CD2 | −1.19 | 25.19 | −0.31 | 13.61 |
| 39 | LEU | C | −3.94 | 28.53 | −0.01 | 13.98 |
| 39 | LEU | O | −3.63 | 28.91 | 1.11 | 13.90 |
| 40 | ASP | N | −4.65 | 29.27 | −0.86 | 14.04 |
| 40 | ASP | CA | −5.09 | 30.61 | −0.51 | 14.16 |
| 40 | ASP | CB | −5.63 | 31.31 | −1.76 | 14.31 |
| 40 | ASP | CG | −5.79 | 32.81 | −1.57 | 14.39 |
| 40 | ASP | OD1 | −4.78 | 33.51 | −1.34 | 14.45 |
| 40 | ASP | OD2 | −6.95 | 33.29 | −1.64 | 14.56 |
| 40 | ASP | C | −6.16 | 30.56 | 0.59 | 14.22 |
| 40 | ASP | O | −6.24 | 31.46 | 1.42 | 14.16 |
| 41 | ASN | N | −6.97 | 29.51 | 0.60 | 14.28 |
| 41 | ASN | CA | −8.02 | 29.39 | 1.61 | 14.49 |
| 41 | ASN | CB | −8.93 | 28.19 | 1.32 | 14.41 |
| 41 | ASN | CG | −9.72 | 28.35 | 0.05 | 14.38 |
| 41 | ASN | OD1 | −9.99 | 29.46 | −0.38 | 14.50 |
| 41 | ASN | ND2 | −10.10 | 27.23 | −0.56 | 14.33 |
| 41 | ASN | C | −7.46 | 29.25 | 3.02 | 14.61 |
| 41 | ASN | O | −8.13 | 29.61 | 3.99 | 14.66 |
| 42 | ILE | N | −6.25 | 28.73 | 3.12 | 14.83 |
| 42 | ILE | CA | −5.61 | 28.58 | 4.42 | 14.92 |
| 42 | ILE | CB | −4.29 | 27.81 | 4.32 | 14.87 |
| 42 | ILE | CG2 | −3.60 | 27.75 | 5.67 | 14.92 |
| 42 | ILE | CG1 | −4.55 | 26.40 | 3.79 | 14.81 |
| 42 | ILE | CD1 | −3.29 | 25.63 | 3.48 | 14.72 |
| 42 | ILE | C | −5.35 | 29.98 | 4.97 | 15.07 |
| 42 | ILE | O | −5.57 | 30.23 | 6.16 | 15.08 |
| 43 | ALA | N | −4.91 | 30.89 | 4.11 | 15.25 |
| 43 | ALA | CA | −4.64 | 32.25 | 4.54 | 15.48 |
| 43 | ALA | CB | −3.99 | 33.05 | 3.40 | 15.40 |
| 43 | ALA | C | −5.95 | 32.91 | 4.97 | 15.67 |
| 43 | ALA | O | −6.00 | 33.59 | 5.99 | 15.74 |
| 44 | LYS | N | −7.01 | 32.70 | 4.19 | 15.93 |
| 44 | LYS | CA | −8.32 | 33.28 | 4.51 | 16.17 |
| 44 | LYS | CB | −9.36 | 32.84 | 3.49 | 16.28 |
| 44 | LYS | CG | −10.78 | 33.29 | 3.82 | 16.45 |
| 44 | LYS | CD | −11.77 | 32.81 | 2.77 | 16.76 |
| 44 | LYS | CE | −13.18 | 33.30 | 3.07 | 16.99 |
| 44 | LYS | NZ | −13.29 | 34.79 | 3.02 | 17.35 |
| 44 | LYS | C | −8.77 | 32.87 | 5.92 | 16.35 |
| 44 | LYS | O | −9.24 | 33.69 | 6.70 | 16.37 |
| 45 | GLU | N | −8.61 | 31.59 | 6.23 | 16.65 |
| 45 | GLU | CA | −9.00 | 31.06 | 7.54 | 16.98 |
| 45 | GLU | CB | −9.00 | 29.53 | 7.52 | 17.25 |
| 45 | GLU | CG | −10.07 | 28.91 | 6.62 | 17.69 |
| 45 | GLU | CD | −11.46 | 29.50 | 6.85 | 17.93 |
| 45 | GLU | OE1 | −11.97 | 29.42 | 7.99 | 18.09 |
| 45 | GLU | OE2 | −12.05 | 30.04 | 5.88 | 18.15 |
| 45 | GLU | C | −8.09 | 31.55 | 8.66 | 17.07 |
| 45 | GLU | O | −8.57 | 31.95 | 9.72 | 17.12 |
| 46 | LEU | N | −6.78 | 31.52 | 8.44 | 17.15 |
| 46 | LEU | CA | −5.86 | 31.99 | 9.47 | 17.23 |
| 46 | LEU | CB | −4.41 | 31.65 | 9.11 | 17.24 |
| 46 | LEU | CG | −4.04 | 30.16 | 9.22 | 17.35 |
| 46 | LEU | CD1 | −2.65 | 29.94 | 8.68 | 17.41 |
| 46 | LEU | CD2 | −4.14 | 29.71 | 10.67 | 17.46 |
| 46 | LEU | C | −6.00 | 33.49 | 9.71 | 17.26 |
| 46 | LEU | O | −5.94 | 33.94 | 10.86 | 17.31 |
| 47 | PHE | N | −6.19 | 34.27 | 8.65 | 17.24 |
| 47 | PHE | CA | −6.35 | 35.71 | 8.82 | 17.29 |
| 47 | PHE | CB | −6.52 | 36.44 | 7.47 | 17.23 |
| 47 | PHE | CG | −5.25 | 36.59 | 6.67 | 17.23 |
| 47 | PHE | CD1 | −4.00 | 36.58 | 7.29 | 17.13 |
| 47 | PHE | CD2 | −5.31 | 36.78 | 5.29 | 17.08 |
| 47 | PHE | CE1 | −2.83 | 36.75 | 6.54 | 17.14 |
| 47 | PHE | CE2 | −4.15 | 36.95 | 4.53 | 17.19 |
| 47 | PHE | CZ | −2.90 | 36.93 | 5.16 | 17.09 |
| 47 | PHE | C | −7.58 | 35.98 | 9.70 | 17.45 |
| 47 | PHE | O | −7.53 | 36.78 | 10.63 | 17.32 |
| 48 | GLU | N | −8.69 | 35.31 | 9.40 | 17.65 |
| 48 | GLU | CA | −9.91 | 35.49 | 10.18 | 17.88 |
| 48 | GLU | CB | −11.06 | 34.68 | 9.56 | 18.04 |
| 48 | GLU | CG | −12.38 | 34.74 | 10.33 | 18.44 |
| 48 | GLU | CD | −12.91 | 36.14 | 10.55 | 18.66 |
| 48 | GLU | OE1 | −12.67 | 37.02 | 9.69 | 18.90 |
| 48 | GLU | OE2 | −13.58 | 36.37 | 11.60 | 18.86 |
| 48 | GLU | C | −9.68 | 35.06 | 11.62 | 17.94 |
| 48 | GLU | O | −10.20 | 35.67 | 12.56 | 17.98 |
| 49 | GLU | N | −8.87 | 34.03 | 11.81 | 17.99 |
| 49 | GLU | CA | −8.57 | 33.53 | 13.15 | 18.12 |
| 49 | GLU | CB | −7.87 | 32.18 | 13.06 | 18.41 |
| 49 | GLU | CG | −7.23 | 31.73 | 14.36 | 18.92 |
| 49 | GLU | CD | −6.67 | 30.32 | 14.28 | 19.22 |
| 49 | GLU | OE1 | −6.08 | 29.96 | 13.23 | 19.52 |
| 49 | GLU | OE2 | −6.82 | 29.58 | 15.27 | 19.44 |
| 49 | GLU | C | −7.71 | 34.50 | 13.96 | 18.04 |
| 49 | GLU | O | −7.88 | 34.63 | 15.18 | 17.91 |
| 50 | TYR | N | −6.79 | 35.17 | 13.29 | 17.86 |
| 50 | TYR | CA | −5.90 | 36.10 | 13.97 | 17.83 |
| 50 | TYR | CB | −4.49 | 35.95 | 13.43 | 17.91 |
| 50 | TYR | CG | −3.87 | 34.63 | 13.78 | 18.11 |
| 50 | TYR | CD1 | −3.57 | 34.32 | 15.11 | 18.16 |
| 50 | TYR | CE1 | −2.96 | 33.11 | 15.44 | 18.23 |
| 50 | TYR | CD2 | −3.55 | 33.70 | 12.80 | 18.11 |
| 50 | TYR | CE2 | −2.94 | 32.49 | 13.12 | 18.19 |
| 50 | TYR | CZ | −2.64 | 32.20 | 14.44 | 18.23 |
| 50 | TYR | OH | −2.00 | 31.03 | 14.76 | 18.38 |
| 50 | TYR | C | −6.32 | 37.55 | 13.90 | 17.67 |
| 50 | TYR | O | −5.56 | 38.44 | 14.28 | 17.65 |
| 51 | GLY | N | −7.53 | 37.79 | 13.40 | 17.53 |
| 51 | GLY | CA | −8.03 | 39.15 | 13.30 | 17.33 |
| 51 | GLY | C | −7.27 | 40.02 | 12.32 | 17.21 |
| 51 | GLY | O | −7.21 | 41.24 | 12.50 | 17.19 |
| 52 | ALA | N | −6.70 | 39.41 | 11.29 | 17.02 |
| 52 | ALA | CA | −5.96 | 40.17 | 10.30 | 16.91 |
| 52 | ALA | CB | −4.57 | 39.57 | 10.09 | 16.88 |
| 52 | ALA | C | −6.71 | 40.21 | 8.98 | 16.86 |
| 52 | ALA | O | −7.67 | 39.46 | 8.76 | 16.70 |
| 53 | ILE | N | −6.26 | 41.08 | 8.10 | 16.87 |
| 53 | ILE | CA | −6.88 | 41.23 | 6.80 | 17.02 |
| 53 | ILE | CB | −7.69 | 42.54 | 6.74 | 17.18 |
| 53 | ILE | CG2 | −8.16 | 42.79 | 5.33 | 17.31 |
| 53 | ILE | CG1 | −8.87 | 42.45 | 7.71 | 17.44 |
| 53 | ILE | CD1 | −9.86 | 41.35 | 7.36 | 17.57 |
| 53 | ILE | C | −5.84 | 41.24 | 5.71 | 16.94 |
| 53 | ILE | O | −4.76 | 41.82 | 5.86 | 16.82 |
| 54 | SER | N | −6.17 | 40.58 | 4.60 | 16.99 |
| 54 | SER | CA | −5.28 | 40.50 | 3.45 | 17.08 |
| 54 | SER | CB | −5.94 | 39.69 | 2.33 | 16.90 |
| 54 | SER | OG | −5.23 | 39.85 | 1.12 | 16.41 |
| 54 | SER | C | −4.91 | 41.88 | 2.93 | 17.32 |
| 54 | SER | O | −5.78 | 42.71 | 2.68 | 17.26 |
| 55 | ALA | N | −3.61 | 42.12 | 2.75 | 17.63 |
| 55 | ALA | CA | −3.14 | 43.41 | 2.24 | 18.01 |
| 55 | ALA | CB | −1.66 | 43.59 | 2.53 | 17.90 |
| 55 | ALA | C | −3.41 | 43.53 | 0.74 | 18.28 |
| 55 | ALA | O | −3.81 | 44.59 | 0.27 | 18.31 |
| 56 | PRO | N | −3.18 | 42.45 | −0.02 | 18.54 |
| 56 | PRO | CD | −2.46 | 41.19 | 0.28 | 18.58 |
| 56 | PRO | CA | −3.44 | 42.57 | −1.46 | 18.80 |
| 56 | PRO | CB | −3.06 | 41.20 | −2.00 | 18.79 |
| 56 | PRO | CG | −1.96 | 40.77 | −1.08 | 18.68 |
| 56 | PRO | C | −4.91 | 42.91 | −1.75 | 19.07 |
| 56 | PRO | O | −5.20 | 43.70 | −2.64 | 19.17 |
| 57 | ILE | N | −5.83 | 42.33 | −0.98 | 19.37 |
| 57 | ILE | CA | −7.25 | 42.60 | −1.20 | 19.70 |
| 57 | ILE | CB | −8.14 | 41.57 | −0.47 | 19.69 |
| 57 | ILE | CG2 | −9.62 | 41.98 | −0.59 | 19.71 |
| 57 | ILE | CG1 | −7.92 | 40.18 | −1.08 | 19.71 |
| 57 | ILE | CD1 | −8.63 | 39.07 | −0.34 | 19.71 |
| 57 | ILE | C | −7.64 | 43.99 | −0.73 | 19.92 |
| 57 | ILE | O | −8.40 | 44.70 | −1.40 | 19.97 |
| 58 | HIS | N | −7.09 | 44.39 | 0.41 | 20.16 |
| 58 | HIS | CA | −7.38 | 45.69 | 1.01 | 20.33 |
| 58 | HIS | CB | −6.96 | 45.67 | 2.48 | 20.34 |
| 58 | HIS | CG | −7.20 | 46.96 | 3.20 | 20.45 |
| 58 | HIS | CD2 | −8.11 | 47.30 | 4.13 | 20.46 |
| 58 | HIS | ND1 | −6.43 | 48.09 | 2.99 | 20.42 |
| 58 | HIS | CE1 | −6.86 | 49.06 | 3.76 | 20.39 |
| 58 | HIS | NE2 | −7.89 | 48.62 | 4.46 | 20.41 |
| 58 | HIS | C | −6.75 | 46.88 | 0.29 | 20.43 |
| 58 | HIS | O | −7.42 | 47.88 | 0.02 | 20.40 |
| 59 | ASP | N | −5.46 | 46.77 | −0.05 | 20.51 |
| 59 | ASP | CA | −4.74 | 47.85 | −0.73 | 20.62 |
| 59 | ASP | CB | −3.24 | 47.77 | −0.41 | 20.68 |
| 59 | ASP | CG | −2.91 | 48.16 | 1.03 | 20.73 |
| 59 | ASP | OD1 | −3.56 | 47.64 | 1.96 | 20.81 |
| 59 | ASP | OD2 | −1.98 | 48.97 | 1.24 | 20.80 |
| 59 | ASP | C | −4.90 | 47.87 | −2.25 | 20.68 |
| 59 | ASP | O | −5.13 | 48.93 | −2.84 | 20.80 |
| 60 | GLU | N | −4.77 | 46.71 | −2.89 | 20.72 |
| 60 | GLU | CA | −4.85 | 46.63 | −4.36 | 20.69 |
| 60 | GLU | CB | −3.64 | 45.86 | −4.89 | 20.92 |
| 60 | GLU | CG | −2.32 | 46.09 | −4.15 | 21.32 |
| 60 | GLU | CD | −1.73 | 47.48 | −4.33 | 21.60 |
| 60 | GLU | OE1 | −1.55 | 47.93 | −5.49 | 21.88 |
| 60 | GLU | OE2 | −1.42 | 48.13 | −3.30 | 21.74 |
| 60 | GLU | C | −6.12 | 45.98 | −4.91 | 20.50 |
| 60 | GLU | O | −6.30 | 45.89 | −6.13 | 20.56 |
| 61 | ASN | N | −7.00 | 45.54 | −4.03 | 20.25 |
| 61 | ASN | CA | −8.22 | 44.86 | −4.46 | 19.98 |
| 61 | ASN | CB | −9.14 | 45.81 | −5.24 | 20.16 |
| 61 | ASN | CG | −10.53 | 45.24 | −5.43 | 20.17 |
| 61 | ASN | OD1 | −10.95 | 44.36 | −4.68 | 20.33 |
| 61 | ASN | ND2 | −11.25 | 45.74 | −6.42 | 20.31 |
| 61 | ASN | C | −7.83 | 43.67 | −5.32 | 19.73 |
| 61 | ASN | O | −8.51 | 43.32 | −6.28 | 19.74 |
| 62 | PHE | N | −6.71 | 43.05 | −4.94 | 19.36 |
| 62 | PHE | CA | −6.18 | 41.88 | −5.62 | 18.95 |
| 62 | PHE | CB | −4.80 | 41.52 | −5.03 | 18.93 |
| 62 | PHE | CG | −4.10 | 40.39 | −5.75 | 18.92 |
| 62 | PHE | CD1 | −3.39 | 40.63 | −6.92 | 18.93 |
| 62 | PHE | CD2 | −4.13 | 39.09 | −5.23 | 18.93 |
| 62 | PHE | CE1 | −2.71 | 39.59 | −7.57 | 18.90 |
| 62 | PHE | CE2 | −3.46 | 38.05 | −5.88 | 18.89 |
| 62 | PHE | CZ | −2.75 | 38.30 | −7.05 | 18.88 |
| 62 | PHE | C | −7.14 | 40.73 | −5.41 | 18.61 |
| 62 | PHE | O | −7.66 | 40.52 | −4.31 | 18.51 |
| 63 | PRO | N | −7.42 | 39.97 | −6.47 | 18.30 |
| 63 | PRO | CD | −6.99 | 40.22 | −7.86 | 18.33 |
| 63 | PRO | CA | −8.33 | 38.83 | −6.40 | 18.07 |
| 63 | PRO | CB | −8.63 | 38.54 | −7.87 | 18.18 |
| 63 | PRO | CG | −7.34 | 38.93 | −8.55 | 18.26 |
| 63 | PRO | C | −7.65 | 37.65 | −5.70 | 17.71 |
| 63 | PRO | O | −7.32 | 36.65 | −6.34 | 17.70 |
| 64 | GLY | N | −7.45 | 37.77 | −4.39 | 17.34 |
| 64 | GLY | CA | −6.82 | 36.69 | −3.65 | 16.84 |
| 64 | GLY | C | −6.29 | 37.13 | −2.29 | 16.44 |
| 64 | GLY | O | −5.88 | 38.27 | −2.12 | 16.41 |
| 65 | GLN | N | −6.31 | 36.22 | −1.32 | 16.13 |
| 65 | GLN | CA | −5.83 | 36.54 | 0.01 | 15.75 |
| 65 | GLN | CB | −6.07 | 35.37 | 0.96 | 15.78 |
| 65 | GLN | CG | −7.53 | 35.01 | 1.20 | 16.03 |
| 65 | GLN | CD | −8.30 | 36.09 | 1.94 | 16.19 |
| 65 | GLN | OE1 | −7.73 | 36.82 | 2.77 | 16.25 |
| 65 | GLN | NE2 | −9.59 | 36.20 | 1.65 | 16.19 |
| 65 | GLN | C | −4.33 | 36.83 | −0.05 | 15.43 |
| 65 | GLN | O | −3.84 | 37.75 | 0.60 | 15.38 |
| 66 | THR | N | −3.63 | 36.04 | −0.85 | 15.13 |
| 66 | THR | CA | −2.18 | 36.16 | −1.01 | 14.82 |
| 66 | THR | CB | −1.47 | 35.04 | −0.25 | 14.86 |
| 66 | THR | OG1 | −1.82 | 33.79 | −0.84 | 14.85 |
| 66 | THR | CG2 | −1.87 | 35.04 | 1.22 | 14.77 |
| 66 | THR | C | −1.77 | 36.01 | −2.48 | 14.65 |
| 66 | THR | O | −2.62 | 35.77 | −3.34 | 14.73 |
| 67 | CYS | N | −0.47 | 36.12 | −2.74 | 14.43 |
| 67 | CYS | CA | 0.08 | 35.97 | −4.08 | 14.14 |
| 67 | CYS | CB | 1.04 | 37.12 | −4.40 | 14.24 |
| 67 | CYS | SG | 0.24 | 38.73 | −4.58 | 14.54 |
| 67 | CYS | C | 0.84 | 34.65 | −4.15 | 13.93 |
| 67 | CYS | O | 1.75 | 34.41 | −3.36 | 13.81 |
| 68 | ILE | N | 0.46 | 33.79 | −5.09 | 13.74 |
| 68 | ILE | CA | 1.11 | 32.49 | −5.27 | 13.59 |
| 68 | ILE | CB | 0.14 | 31.33 | −4.95 | 13.56 |
| 68 | ILE | CG2 | 0.86 | 29.99 | −5.11 | 13.63 |
| 68 | ILE | CG1 | −0.36 | 31.46 | −3.51 | 13.59 |
| 68 | ILE | CD1 | −1.49 | 30.51 | −3.19 | 13.73 |
| 68 | ILE | C | 1.52 | 32.40 | −6.73 | 13.51 |
| 68 | ILE | O | 0.67 | 32.35 | −7.62 | 13.59 |
| 69 | SER | N | 2.82 | 32.37 | −6.96 | 13.39 |
| 69 | SER | CA | 3.40 | 32.34 | −8.30 | 13.25 |
| 69 | SER | CB | 4.37 | 33.51 | −8.43 | 13.19 |
| 69 | SER | OG | 3.75 | 34.72 | −7.98 | 13.10 |
| 69 | SER | C | 4.13 | 31.02 | −8.54 | 13.23 |
| 69 | SER | O | 4.99 | 30.63 | −7.75 | 13.18 |
| 70 | VAL | N | 3.81 | 30.33 | −9.64 | 13.19 |
| 70 | VAL | CA | 4.43 | 29.04 | −9.93 | 13.24 |
| 70 | VAL | CB | 3.38 | 27.90 | −9.94 | 13.33 |
| 70 | VAL | CG1 | 4.01 | 26.57 | −10.34 | 13.39 |
| 70 | VAL | CG2 | 2.73 | 27.80 | −8.57 | 13.35 |
| 70 | VAL | C | 5.22 | 28.99 | −11.23 | 13.32 |
| 70 | VAL | O | 4.75 | 29.45 | −12.26 | 13.25 |
| 71 | ASN | N | 6.43 | 28.43 | −11.17 | 13.35 |
| 71 | ASN | CA | 7.30 | 28.27 | −12.33 | 13.56 |
| 71 | ASN | CB | 6.81 | 27.10 | −13.19 | 13.48 |
| 71 | ASN | CG | 6.78 | 25.78 | −12.42 | 13.47 |
| 71 | ASN | OD1 | 7.59 | 25.55 | −11.54 | 13.42 |
| 71 | ASN | ND2 | 5.84 | 24.92 | −12.78 | 13.51 |
| 71 | ASN | C | 7.54 | 29.50 | −13.21 | 13.75 |
| 71 | ASN | O | 8.45 | 30.29 | −12.95 | 13.68 |
| 72 | GLU | N | 6.75 | 29.66 | −14.28 | 13.97 |
| 72 | GLU | CA | 6.96 | 30.79 | −15.16 | 14.22 |
| 72 | GLU | CB | 6.20 | 30.61 | −16.49 | 14.17 |
| 72 | GLU | CG | 4.69 | 30.83 | −16.46 | 14.32 |
| 72 | GLU | CD | 3.93 | 29.72 | −15.76 | 14.34 |
| 72 | GLU | OE1 | 4.44 | 28.58 | −15.69 | 14.40 |
| 72 | GLU | OE2 | 2.81 | 29.99 | −15.30 | 14.51 |
| 72 | GLU | C | 6.57 | 32.11 | −14.50 | 14.31 |
| 72 | GLU | O | 7.02 | 33.18 | −14.89 | 14.39 |
| 73 | GLU | N | 5.72 | 32.03 | −13.48 | 14.45 |
| 73 | GLU | CA | 5.30 | 33.23 | −12.76 | 14.57 |
| 73 | GLU | CB | 3.94 | 33.02 | −12.13 | 14.70 |
| 73 | GLU | CG | 2.92 | 32.54 | −13.10 | 14.77 |
| 73 | GLU | CD | 1.60 | 32.27 | −12.42 | 14.88 |
| 73 | GLU | OE1 | 1.62 | 31.63 | −11.34 | 14.80 |
| 73 | GLU | OE2 | 0.57 | 32.70 | −12.97 | 14.97 |
| 73 | GLU | C | 6.31 | 33.53 | −11.66 | 14.65 |
| 73 | GLU | O | 6.66 | 32.66 | −10.88 | 14.65 |
| 74 | VAL | N | 6.76 | 34.78 | −11.62 | 14.82 |
| 74 | VAL | CA | 7.75 | 35.23 | −10.65 | 14.95 |
| 74 | VAL | CB | 8.74 | 36.24 | −11.29 | 14.94 |
| 74 | VAL | CG1 | 9.76 | 36.71 | −10.26 | 14.91 |
| 74 | VAL | CG2 | 9.44 | 35.59 | −12.48 | 14.96 |
| 74 | VAL | C | 7.11 | 35.90 | −9.45 | 15.05 |
| 74 | VAL | O | 7.60 | 35.80 | −8.33 | 15.04 |
| 75 | ALA | N | 6.00 | 36.59 | −9.68 | 15.21 |
| 75 | ALA | CA | 5.36 | 37.28 | −8.57 | 15.38 |
| 75 | ALA | CB | 6.21 | 38.49 | −8.17 | 15.28 |
| 75 | ALA | C | 3.95 | 37.73 | −8.94 | 15.47 |
| 75 | ALA | O | 3.60 | 37.79 | −10.11 | 15.49 |
| 76 | HIS | N | 3.16 | 38.02 | −7.91 | 15.64 |
| 76 | HIS | CA | 1.78 | 38.47 | −8.08 | 15.89 |
| 76 | HIS | CB | 1.74 | 39.77 | −8.88 | 16.35 |
| 76 | HIS | CG | 2.41 | 40.92 | −8.21 | 16.73 |
| 76 | HIS | CD2 | 2.27 | 42.26 | −8.40 | 16.99 |
| 76 | HIS | ND1 | 3.41 | 40.77 | −7.28 | 16.99 |
| 76 | HIS | CE1 | 3.87 | 41.96 | −6.92 | 17.05 |
| 76 | HIS | NE2 | 3.19 | 42.88 | −7.58 | 17.22 |
| 76 | HIS | C | 0.88 | 37.43 | −8.75 | 15.89 |
| 76 | HIS | O | −0.10 | 37.79 | −9.39 | 15.82 |
| 77 | GLY | N | 1.23 | 36.15 | −8.60 | 15.84 |
| 77 | GLY | CA | 0.43 | 35.09 | −9.19 | 15.84 |
| 77 | GLY | C | −0.93 | 35.04 | −8.52 | 15.82 |
| 77 | GLY | O | −1.05 | 35.26 | −7.31 | 15.72 |
| 78 | ILE | N | −1.97 | 34.74 | −9.30 | 15.85 |
| 78 | ILE | CA | −3.33 | 34.69 | −8.79 | 15.94 |
| 78 | ILE | CB | −4.33 | 35.20 | −9.87 | 16.19 |
| 78 | ILE | CG2 | −5.73 | 35.27 | −9.31 | 16.15 |
| 78 | ILE | CG1 | −3.91 | 36.60 | −10.34 | 16.30 |
| 78 | ILE | CD1 | −4.73 | 37.11 | −11.51 | 16.42 |
| 78 | ILE | C | −3.75 | 33.27 | −8.39 | 15.98 |
| 78 | ILE | O | −3.65 | 32.35 | −9.19 | 15.92 |
| 79 | PRO | N | −4.19 | 33.09 | −7.13 | 16.02 |
| 79 | PRO | CD | −4.17 | 34.04 | −6.00 | 16.03 |
| 79 | PRO | CA | −4.62 | 31.76 | −6.67 | 15.97 |
| 79 | PRO | CB | −5.06 | 32.02 | −5.24 | 16.04 |
| 79 | PRO | CG | −4.12 | 33.12 | −4.80 | 16.00 |
| 79 | PRO | C | −5.76 | 31.30 | −7.57 | 16.02 |
| 79 | PRO | O | −6.66 | 32.09 | −7.89 | 16.05 |
| 80 | SER | N | −5.75 | 30.04 | −7.96 | 16.04 |
| 80 | SER | CA | −6.78 | 29.54 | −8.86 | 16.13 |
| 80 | SER | CB | −6.48 | 30.07 | −10.26 | 16.17 |
| 80 | SER | OG | −5.24 | 29.55 | −10.72 | 16.26 |
| 80 | SER | C | −6.81 | 28.02 | −8.90 | 16.20 |
| 80 | SER | O | −6.23 | 27.36 | −8.04 | 16.22 |
| 81 | LYS | N | −7.47 | 27.48 | −9.91 | 16.18 |
| 81 | LYS | CA | −7.58 | 26.03 | −10.06 | 16.14 |
| 81 | LYS | CB | −8.66 | 25.70 | −11.09 | 16.29 |
| 81 | LYS | CG | −10.01 | 26.35 | −10.79 | 16.59 |
| 81 | LYS | CD | −10.85 | 26.52 | −12.05 | 16.89 |
| 81 | LYS | CE | −12.16 | 27.25 | −11.74 | 17.00 |
| 81 | LYS | NZ | −12.99 | 27.54 | −12.94 | 17.15 |
| 81 | LYS | C | −6.26 | 25.37 | −10.45 | 15.94 |
| 81 | LYS | O | −6.19 | 24.15 | −10.59 | 15.98 |
| 82 | ARG | N | −5.22 | 26.18 | −10.65 | 15.73 |
| 82 | ARG | CA | −3.92 | 25.63 | −11.02 | 15.55 |
| 82 | ARG | CB | −2.87 | 26.75 | −11.10 | 15.55 |
| 82 | ARG | CG | −1.47 | 26.24 | −11.35 | 15.43 |
| 82 | ARG | CD | −0.56 | 27.33 | −11.85 | 15.54 |
| 82 | ARG | NE | 0.66 | 26.78 | −12.43 | 15.55 |
| 82 | ARG | CZ | 1.54 | 27.50 | −13.13 | 15.62 |
| 82 | ARG | NH1 | 1.33 | 28.79 | −13.33 | 15.58 |
| 82 | ARG | NH2 | 2.62 | 26.92 | −13.63 | 15.58 |
| 82 | ARG | C | −3.47 | 24.59 | −9.98 | 15.46 |
| 82 | ARG | O | −3.42 | 24.89 | −8.79 | 15.34 |
| 83 | VAL | N | −3.16 | 23.38 | −10.43 | 15.34 |
| 83 | VAL | CA | −2.74 | 22.33 | −9.50 | 15.21 |
| 83 | VAL | CB | −3.25 | 20.93 | −9.93 | 15.31 |
| 83 | VAL | CG1 | −2.54 | 20.45 | −11.18 | 15.43 |
| 83 | VAL | CG2 | −3.05 | 19.94 | −8.80 | 15.39 |
| 83 | VAL | C | −1.22 | 22.25 | −9.36 | 15.04 |
| 83 | VAL | O | −0.49 | 22.32 | −10.35 | 14.99 |
| 84 | ILE | N | −0.75 | 22.10 | −8.13 | 14.81 |
| 84 | ILE | CA | 0.69 | 21.99 | −7.87 | 14.67 |
| 84 | ILE | CB | 1.01 | 22.15 | −6.36 | 14.60 |
| 84 | ILE | CG2 | 2.47 | 21.84 | −6.11 | 14.51 |
| 84 | ILE | CG1 | 0.67 | 23.58 | −5.90 | 14.63 |
| 84 | ILE | CD1 | 1.49 | 24.66 | −6.57 | 14.70 |
| 84 | ILE | C | 1.18 | 20.62 | −8.33 | 14.58 |
| 84 | ILE | O | 0.54 | 19.60 | −8.08 | 14.55 |
| 85 | ARG | N | 2.33 | 20.61 | −8.99 | 14.53 |
| 85 | ARG | CA | 2.92 | 19.37 | −9.50 | 14.51 |
| 85 | ARG | CB | 2.99 | 19.43 | −11.03 | 14.88 |
| 85 | ARG | CG | 1.64 | 19.53 | −11.70 | 15.43 |
| 85 | ARG | CD | 0.82 | 18.27 | −11.47 | 15.80 |
| 85 | ARG | NE | 1.46 | 17.12 | −12.10 | 16.29 |
| 85 | ARG | CZ | 1.21 | 16.72 | −13.35 | 16.50 |
| 85 | ARG | NH1 | 0.33 | 17.37 | −14.10 | 16.70 |
| 85 | ARG | NH2 | 1.87 | 15.68 | −13.85 | 16.75 |
| 85 | ARG | C | 4.33 | 19.13 | −8.96 | 14.35 |
| 85 | ARG | O | 5.02 | 20.06 | −8.56 | 14.10 |
| 86 | GLU | N | 4.75 | 17.87 | −8.95 | 14.10 |
| 86 | GLU | CA | 6.08 | 17.52 | −8.49 | 14.00 |
| 86 | GLU | CB | 6.33 | 16.04 | −8.77 | 14.17 |
| 86 | GLU | CG | 7.68 | 15.51 | −8.32 | 14.45 |
| 86 | GLU | CD | 7.80 | 15.34 | −6.83 | 14.55 |
| 86 | GLU | OE1 | 6.78 | 15.44 | −6.11 | 14.66 |
| 86 | GLU | OE2 | 8.93 | 15.09 | −6.36 | 14.63 |
| 86 | GLU | C | 7.09 | 18.38 | −9.25 | 13.76 |
| 86 | GLU | O | 6.95 | 18.60 | −10.45 | 13.74 |
| 87 | GLY | N | 8.11 | 18.88 | −8.54 | 13.49 |
| 87 | GLY | CA | 9.13 | 19.71 | −9.17 | 13.16 |
| 87 | GLY | C | 8.80 | 21.19 | −9.34 | 12.97 |
| 87 | GLY | O | 9.65 | 21.97 | −9.78 | 12.91 |
| 88 | ASP | N | 7.59 | 21.59 | −8.98 | 12.75 |
| 88 | ASP | CA | 7.21 | 23.01 | −9.14 | 12.65 |
| 88 | ASP | CB | 5.73 | 23.24 | −8.79 | 12.74 |
| 88 | ASP | CG | 4.80 | 22.89 | −9.94 | 12.76 |
| 88 | ASP | OD1 | 5.29 | 22.49 | −11.00 | 12.87 |
| 88 | ASP | OD2 | 3.56 | 23.03 | −9.76 | 12.81 |
| 88 | ASP | C | 8.06 | 23.94 | −8.28 | 12.41 |
| 88 | ASP | O | 8.47 | 23.59 | −7.17 | 12.45 |
| 89 | LEU | N | 8.32 | 25.13 | −8.81 | 12.24 |
| 89 | LEU | CA | 9.06 | 26.15 | −8.08 | 11.94 |
| 89 | LEU | CB | 10.11 | 26.80 | −8.97 | 11.90 |
| 89 | LEU | CG | 10.82 | 28.02 | −8.37 | 11.89 |
| 89 | LEU | CD1 | 11.40 | 27.69 | −6.98 | 11.89 |
| 89 | LEU | CD2 | 11.91 | 28.47 | −9.32 | 11.91 |
| 89 | LEU | C | 7.98 | 27.15 | −7.69 | 11.92 |
| 89 | LEU | O | 7.46 | 27.88 | −8.54 | 11.86 |
| 90 | VAL | N | 7.64 | 27.17 | −6.41 | 11.81 |
| 90 | VAL | CA | 6.58 | 28.02 | −5.88 | 11.74 |
| 90 | VAL | CB | 5.58 | 27.16 | −5.07 | 11.66 |
| 90 | VAL | CG1 | 4.41 | 28.01 | −4.57 | 11.68 |
| 90 | VAL | CG2 | 5.08 | 26.01 | −5.93 | 11.69 |
| 90 | VAL | C | 7.03 | 29.18 | −4.98 | 11.69 |
| 90 | VAL | O | 7.86 | 29.01 | −4.10 | 11.49 |
| 91 | ASN | N | 6.45 | 30.35 | −5.23 | 11.78 |
| 91 | ASN | CA | 6.71 | 31.56 | −4.45 | 11.80 |
| 91 | ASN | CB | 7.19 | 32.70 | −5.34 | 12.23 |
| 91 | ASN | CG | 7.51 | 33.97 | −4.54 | 12.73 |
| 91 | ASN | OD1 | 6.70 | 34.42 | −3.72 | 12.94 |
| 91 | ASN | ND2 | 8.69 | 34.55 | −4.79 | 13.23 |
| 91 | ASN | C | 5.37 | 31.94 | −3.82 | 11.68 |
| 91 | ASN | O | 4.38 | 32.18 | −4.52 | 11.62 |
| 92 | ILE | N | 5.35 | 31.98 | −2.49 | 11.43 |
| 92 | ILE | CA | 4.15 | 32.34 | −1.74 | 11.31 |
| 92 | ILE | CB | 3.79 | 31.26 | −0.70 | 11.29 |
| 92 | ILE | CG2 | 2.67 | 31.75 | 0.22 | 11.25 |
| 92 | ILE | CG1 | 3.40 | 29.96 | −1.42 | 11.35 |
| 92 | ILE | CD1 | 3.06 | 28.79 | −0.47 | 11.33 |
| 92 | ILE | C | 4.48 | 33.64 | −1.04 | 11.36 |
| 92 | ILE | O | 5.44 | 33.72 | −0.28 | 11.14 |
| 93 | ASP | N | 3.68 | 34.67 | −1.31 | 11.37 |
| 93 | ASP | CA | 3.91 | 35.98 | −0.71 | 11.37 |
| 93 | ASP | CB | 4.19 | 37.01 | −1.81 | 11.47 |
| 93 | ASP | CG | 4.78 | 38.30 | −1.27 | 11.61 |
| 93 | ASP | OD1 | 4.29 | 38.78 | −0.23 | 11.73 |
| 93 | ASP | OD2 | 5.72 | 38.82 | −1.90 | 11.96 |
| 93 | ASP | C | 2.70 | 36.37 | 0.11 | 11.35 |
| 93 | ASP | O | 1.59 | 36.50 | −0.41 | 11.29 |
| 94 | VAL | N | 2.92 | 36.55 | 1.41 | 11.32 |
| 94 | VAL | CA | 1.86 | 36.88 | 2.34 | 11.43 |
| 94 | VAL | CB | 1.82 | 35.84 | 3.50 | 11.42 |
| 94 | VAL | CG1 | 0.57 | 36.03 | 4.34 | 11.32 |
| 94 | VAL | CG2 | 1.88 | 34.42 | 2.93 | 11.50 |
| 94 | VAL | C | 2.06 | 38.27 | 2.96 | 11.50 |
| 94 | VAL | O | 3.12 | 38.56 | 3.51 | 11.50 |
| 95 | SER | N | 1.05 | 39.12 | 2.85 | 11.58 |
| 95 | SER | CA | 1.11 | 40.45 | 3.46 | 11.61 |
| 95 | SER | CB | 1.59 | 41.53 | 2.46 | 11.67 |
| 95 | SER | OG | 0.74 | 41.66 | 1.34 | 11.64 |
| 95 | SER | C | −0.29 | 40.76 | 3.97 | 11.72 |
| 95 | SER | O | −1.29 | 40.39 | 3.35 | 11.60 |
| 96 | ALA | N | −0.37 | 41.41 | 5.12 | 11.79 |
| 96 | ALA | CA | −1.66 | 41.71 | 5.73 | 11.94 |
| 96 | ALA | CB | −2.16 | 40.48 | 6.49 | 11.82 |
| 96 | ALA | C | −1.52 | 42.88 | 6.69 | 12.08 |
| 96 | ALA | O | −0.44 | 43.41 | 6.87 | 12.16 |
| 97 | LEU | N | −2.64 | 43.26 | 7.29 | 12.30 |
| 97 | LEU | CA | −2.64 | 44.35 | 8.25 | 12.48 |
| 97 | LEU | CB | −3.13 | 45.66 | 7.60 | 12.53 |
| 97 | LEU | CG | −4.51 | 45.66 | 6.94 | 12.61 |
| 97 | LEU | CD1 | −5.03 | 47.10 | 6.83 | 12.64 |
| 97 | LEU | CD2 | −4.44 | 45.03 | 5.57 | 12.64 |
| 97 | LEU | C | −3.54 | 44.01 | 9.43 | 12.56 |
| 97 | LEU | O | −4.50 | 43.25 | 9.29 | 12.54 |
| 98 | LYS | N | −3.21 | 44.54 | 10.59 | 12.67 |
| 98 | LYS | CA | −4.01 | 44.33 | 11.79 | 12.86 |
| 98 | LYS | CB | −3.54 | 43.10 | 12.58 | 12.93 |
| 98 | LYS | CG | −4.31 | 42.88 | 13.90 | 12.99 |
| 98 | LYS | CD | −3.88 | 41.59 | 14.59 | 13.13 |
| 98 | LYS | CE | −4.65 | 41.36 | 15.88 | 13.25 |
| 98 | LYS | NZ | −4.30 | 40.03 | 16.49 | 13.32 |
| 98 | LYS | C | −3.85 | 45.59 | 12.62 | 13.01 |
| 98 | LYS | O | −2.74 | 46.08 | 12.82 | 12.96 |
| 99 | ASN | N | −4.98 | 46.16 | 13.05 | 13.13 |
| 99 | ASN | CA | −4.97 | 47.38 | 13.83 | 13.29 |
| 99 | ASN | CB | −4.42 | 47.10 | 15.23 | 13.37 |
| 99 | ASN | CG | −5.32 | 46.17 | 16.02 | 13.41 |
| 99 | ASN | OD1 | −6.54 | 46.33 | 16.01 | 13.54 |
| 99 | ASN | ND2 | −4.74 | 45.19 | 16.69 | 13.45 |
| 99 | ASN | C | −4.18 | 48.52 | 13.18 | 13.26 |
| 99 | ASN | O | −3.60 | 49.36 | 13.87 | 13.41 |
| 100 | GLY | N | −4.15 | 48.53 | 11.85 | 13.22 |
| 100 | GLY | CA | −3.46 | 49.58 | 11.12 | 13.11 |
| 100 | GLY | C | −1.99 | 49.36 | 10.82 | 12.97 |
| 100 | GLY | O | −1.34 | 50.23 | 10.25 | 13.07 |
| 101 | TYR | N | −1.45 | 48.20 | 11.20 | 12.75 |
| 101 | TYR | CA | −0.04 | 47.92 | 10.95 | 12.45 |
| 101 | TYR | CB | 0.69 | 47.75 | 12.27 | 12.65 |
| 101 | TYR | CG | 0.70 | 49.02 | 13.10 | 12.97 |
| 101 | TYR | CD1 | 1.71 | 49.96 | 12.95 | 13.10 |
| 101 | TYR | CE1 | 1.72 | 51.14 | 13.71 | 13.37 |
| 101 | TYR | CD2 | −0.32 | 49.27 | 14.01 | 13.14 |
| 101 | TYR | CE2 | −0.34 | 50.45 | 14.78 | 13.41 |
| 101 | TYR | CZ | 0.69 | 51.37 | 14.62 | 13.48 |
| 101 | TYR | OH | 0.69 | 52.51 | 15.40 | 13.70 |
| 101 | TYR | C | 0.14 | 46.71 | 10.05 | 12.16 |
| 101 | TYR | O | −0.58 | 45.71 | 10.17 | 11.97 |
| 102 | TYR | N | 1.10 | 46.81 | 9.14 | 11.96 |
| 102 | TYR | CA | 1.36 | 45.77 | 8.15 | 11.76 |
| 102 | TYR | CB | 1.47 | 46.41 | 6.76 | 11.76 |
| 102 | TYR | CG | 0.22 | 47.05 | 6.22 | 12.03 |
| 102 | TYR | CD1 | −0.39 | 48.13 | 6.86 | 12.07 |
| 102 | TYR | CE1 | −1.54 | 48.74 | 6.31 | 12.26 |
| 102 | TYR | CD2 | −0.34 | 46.60 | 5.03 | 12.00 |
| 102 | TYR | CE2 | −1.46 | 47.19 | 4.49 | 12.25 |
| 102 | TYR | CZ | −2.06 | 48.26 | 5.12 | 12.29 |
| 102 | TYR | OH | −3.17 | 48.80 | 4.53 | 12.39 |
| 102 | TYR | C | 2.62 | 44.94 | 8.34 | 11.60 |
| 102 | TYR | O | 3.54 | 45.33 | 9.05 | 11.50 |
| 103 | ALA | N | 2.64 | 43.79 | 7.67 | 11.49 |
| 103 | ALA | CA | 3.79 | 42.89 | 7.64 | 11.39 |
| 103 | ALA | CB | 3.61 | 41.72 | 8.60 | 11.42 |
| 103 | ALA | C | 3.84 | 42.39 | 6.19 | 11.35 |
| 103 | ALA | O | 2.82 | 42.37 | 5.49 | 11.34 |
| 104 | ASP | N | 5.02 | 41.99 | 5.73 | 11.30 |
| 104 | ASP | CA | 5.17 | 41.52 | 4.37 | 11.25 |
| 104 | ASP | CB | 5.50 | 42.72 | 3.46 | 11.41 |
| 104 | ASP | CG | 5.57 | 42.37 | 1.99 | 11.55 |
| 104 | ASP | OD1 | 5.20 | 41.23 | 1.62 | 11.59 |
| 104 | ASP | OD2 | 5.97 | 43.25 | 1.21 | 11.51 |
| 104 | ASP | C | 6.29 | 40.49 | 4.35 | 11.15 |
| 104 | ASP | O | 7.43 | 40.79 | 4.73 | 11.03 |
| 105 | THR | N | 5.98 | 39.28 | 3.91 | 11.09 |
| 105 | THR | CA | 6.99 | 38.24 | 3.87 | 10.99 |
| 105 | THR | CB | 7.07 | 37.53 | 5.26 | 11.07 |
| 105 | THR | OG1 | 8.08 | 36.52 | 5.24 | 11.02 |
| 105 | THR | CG2 | 5.74 | 36.93 | 5.64 | 11.10 |
| 105 | THR | C | 6.65 | 37.27 | 2.76 | 10.95 |
| 105 | THR | O | 5.50 | 37.18 | 2.33 | 10.96 |
| 106 | GLY | N | 7.66 | 36.55 | 2.27 | 10.87 |
| 106 | GLY | CA | 7.42 | 35.61 | 1.20 | 10.72 |
| 106 | GLY | C | 8.54 | 34.60 | 1.11 | 10.71 |
| 106 | GLY | O | 9.61 | 34.78 | 1.68 | 10.51 |
| 107 | ILE | N | 8.29 | 33.52 | 0.39 | 10.68 |
| 107 | ILE | CA | 9.31 | 32.49 | 0.25 | 10.67 |
| 107 | ILE | CB | 9.39 | 31.61 | 1.55 | 10.54 |
| 107 | ILE | CG2 | 8.08 | 30.88 | 1.79 | 10.60 |
| 107 | ILE | CG1 | 10.54 | 30.61 | 1.44 | 10.66 |
| 107 | ILE | CD1 | 10.89 | 29.95 | 2.78 | 10.77 |
| 107 | ILE | C | 9.09 | 31.62 | −0.98 | 10.69 |
| 107 | ILE | O | 7.95 | 31.35 | −1.38 | 10.50 |
| 108 | SER | N | 10.20 | 31.25 | −1.61 | 10.70 |
| 108 | SER | CA | 10.18 | 30.38 | −2.78 | 10.85 |
| 108 | SER | CB | 11.02 | 30.96 | −3.93 | 10.76 |
| 108 | SER | OG | 10.46 | 32.15 | −4.45 | 10.72 |
| 108 | SER | C | 10.81 | 29.08 | −2.33 | 11.03 |
| 108 | SER | O | 11.79 | 29.07 | −1.59 | 10.89 |
| 109 | PHE | N | 10.22 | 27.98 | −2.78 | 11.24 |
| 109 | PHE | CA | 10.71 | 26.65 | −2.45 | 11.61 |
| 109 | PHE | CB | 10.07 | 26.15 | −1.15 | 11.60 |
| 109 | PHE | CG | 8.56 | 26.15 | −1.17 | 11.68 |
| 109 | PHE | CD1 | 7.86 | 27.33 | −0.94 | 11.76 |
| 109 | PHE | CD2 | 7.85 | 24.99 | −1.45 | 11.78 |
| 109 | PHE | CE1 | 6.46 | 27.36 | −0.99 | 11.74 |
| 109 | PHE | CE2 | 6.45 | 25.01 | −1.51 | 11.88 |
| 109 | PHE | CZ | 5.76 | 26.20 | −1.28 | 11.77 |
| 109 | PHE | C | 10.36 | 25.71 | −3.60 | 11.82 |
| 109 | PHE | O | 9.56 | 26.07 | −4.48 | 11.67 |
| 110 | VAL | N | 10.97 | 24.54 | −3.60 | 12.16 |
| 110 | VAL | CA | 10.72 | 23.53 | −4.63 | 12.47 |
| 110 | VAL | CB | 12.04 | 22.85 | −5.08 | 12.54 |
| 110 | VAL | CG1 | 11.74 | 21.69 | −6.04 | 12.54 |
| 110 | VAL | CG2 | 12.94 | 23.87 | −5.76 | 12.54 |
| 110 | VAL | C | 9.79 | 22.48 | −4.04 | 12.73 |
| 110 | VAL | O | 9.94 | 22.08 | −2.88 | 12.74 |
| 111 | VAL | N | 8.82 | 22.04 | −4.83 | 12.96 |
| 111 | VAL | CA | 7.88 | 21.02 | −4.38 | 13.20 |
| 111 | VAL | CB | 6.52 | 21.17 | −5.05 | 13.26 |
| 111 | VAL | CG1 | 5.54 | 20.12 | −4.52 | 13.35 |
| 111 | VAL | CG2 | 5.99 | 22.57 | −4.81 | 13.30 |
| 111 | VAL | C | 8.48 | 19.68 | −4.76 | 13.44 |
| 111 | VAL | O | 8.67 | 19.39 | −5.95 | 13.34 |
| 112 | GLY | N | 8.78 | 18.86 | −3.76 | 13.65 |
| 112 | GLY | CA | 9.36 | 17.57 | −4.04 | 14.12 |
| 112 | GLY | C | 10.69 | 17.71 | −4.75 | 14.37 |
| 112 | GLY | O | 11.56 | 18.47 | −4.31 | 14.43 |
| 113 | GLU | N | 10.85 | 17.00 | −5.86 | 14.73 |
| 113 | GLU | CA | 12.09 | 17.05 | −6.62 | 15.11 |
| 113 | GLU | CB | 12.79 | 15.71 | −6.57 | 15.34 |
| 113 | GLU | CG | 13.02 | 15.19 | −5.17 | 15.81 |
| 113 | GLU | CD | 13.53 | 13.77 | −5.17 | 16.07 |
| 113 | GLU | OE1 | 12.82 | 12.88 | −5.71 | 16.38 |
| 113 | GLU | OE2 | 14.63 | 13.54 | −4.63 | 16.29 |
| 113 | GLU | C | 11.86 | 17.43 | −8.08 | 15.24 |
| 113 | GLU | O | 10.88 | 17.02 | −8.69 | 15.23 |
| 114 | SER | N | 12.80 | 18.20 | −8.62 | 15.34 |
| 114 | SER | CA | 12.79 | 18.64 | −10.01 | 15.49 |
| 114 | SER | CB | 12.95 | 20.17 | −10.08 | 15.62 |
| 114 | SER | OG | 13.28 | 20.60 | −11.39 | 15.85 |
| 114 | SER | C | 13.99 | 17.98 | −10.68 | 15.56 |
| 114 | SER | O | 15.00 | 17.73 | −10.03 | 15.53 |
| 115 | ASP | N | 13.88 | 17.70 | −11.98 | 15.71 |
| 115 | ASP | CA | 15.00 | 17.09 | −12.70 | 15.84 |
| 115 | ASP | CB | 14.53 | 16.54 | −14.05 | 16.37 |
| 115 | ASP | CG | 14.12 | 17.63 | −15.00 | 16.79 |
| 115 | ASP | OD1 | 13.53 | 18.61 | −14.52 | 17.18 |
| 115 | ASP | OD2 | 14.37 | 17.50 | −16.22 | 17.31 |
| 115 | ASP | C | 16.09 | 18.13 | −12.94 | 15.69 |
| 115 | ASP | O | 17.19 | 17.79 | −13.38 | 15.64 |
| 116 | ASP | N | 15.78 | 19.40 | −12.68 | 15.48 |
| 116 | ASP | CA | 16.76 | 20.46 | −12.86 | 15.38 |
| 116 | ASP | CB | 16.19 | 21.61 | −13.70 | 15.50 |
| 116 | ASP | CG | 17.28 | 22.57 | −14.19 | 15.55 |
| 116 | ASP | OD1 | 18.36 | 22.61 | −13.57 | 15.62 |
| 116 | ASP | OD2 | 17.04 | 23.29 | −15.19 | 15.61 |
| 116 | ASP | C | 17.14 | 20.98 | −11.48 | 15.27 |
| 116 | ASP | O | 16.32 | 21.59 | −10.80 | 15.21 |
| 117 | PRO | N | 18.39 | 20.76 | −11.07 | 15.13 |
| 117 | PRO | CD | 19.47 | 20.03 | −11.77 | 15.16 |
| 117 | PRO | CA | 18.83 | 21.23 | −9.75 | 15.04 |
| 117 | PRO | CB | 20.20 | 20.55 | −9.59 | 15.12 |
| 117 | PRO | CG | 20.70 | 20.43 | −10.99 | 15.09 |
| 117 | PRO | C | 18.89 | 22.76 | −9.60 | 14.97 |
| 117 | PRO | O | 18.98 | 23.28 | −8.49 | 14.92 |
| 118 | MET | N | 18.80 | 23.48 | −10.72 | 14.94 |
| 118 | MET | CA | 18.84 | 24.95 | −10.73 | 14.94 |
| 118 | MET | CB | 18.83 | 25.47 | −12.17 | 15.14 |
| 118 | MET | CG | 18.72 | 26.99 | −12.30 | 15.53 |
| 118 | MET | SD | 20.20 | 27.88 | −11.68 | 15.88 |
| 118 | MET | CE | 21.33 | 27.62 | −13.04 | 15.90 |
| 118 | MET | C | 17.68 | 25.57 | −9.96 | 14.83 |
| 118 | MET | O | 17.79 | 26.68 | −9.44 | 14.73 |
| 119 | LYS | N | 16.56 | 24.85 | −9.89 | 14.75 |
| 119 | LYS | CA | 15.42 | 25.36 | −9.16 | 14.63 |
| 119 | LYS | CB | 14.23 | 24.40 | −9.32 | 14.60 |
| 119 | LYS | CG | 13.52 | 24.52 | −10.67 | 14.64 |
| 119 | LYS | CD | 12.19 | 23.76 | −10.70 | 14.70 |
| 119 | LYS | CE | 11.37 | 24.10 | −11.94 | 14.76 |
| 119 | LYS | NZ | 10.13 | 23.25 | −12.05 | 14.66 |
| 119 | LYS | C | 15.80 | 25.56 | −7.70 | 14.53 |
| 119 | LYS | O | 15.60 | 26.65 | −7.15 | 14.56 |
| 120 | GLN | N | 16.39 | 24.54 | −7.07 | 14.42 |
| 120 | GLN | CA | 16.80 | 24.64 | −5.67 | 14.29 |
| 120 | GLN | CB | 17.28 | 23.28 | −5.15 | 14.35 |
| 120 | GLN | CG | 17.56 | 23.23 | −3.64 | 14.30 |
| 120 | GLN | CD | 16.40 | 23.76 | −2.81 | 14.26 |
| 120 | GLN | OE1 | 15.28 | 23.28 | −2.90 | 14.27 |
| 120 | GLN | NE2 | 16.68 | 24.77 | −1.97 | 14.18 |
| 120 | GLN | C | 17.91 | 25.68 | −5.52 | 14.22 |
| 120 | GLN | O | 17.91 | 26.46 | −4.57 | 14.28 |
| 121 | LYS | N | 18.85 | 25.69 | −6.47 | 14.09 |
| 121 | LYS | CA | 19.96 | 26.63 | −6.42 | 13.95 |
| 121 | LYS | CB | 20.83 | 26.52 | −7.68 | 14.09 |
| 121 | LYS | CG | 22.05 | 27.42 | −7.62 | 14.24 |
| 121 | LYS | CD | 22.99 | 27.19 | −8.80 | 14.46 |
| 121 | LYS | CE | 24.24 | 28.06 | −8.67 | 14.56 |
| 121 | LYS | NZ | 25.11 | 27.96 | −9.86 | 14.64 |
| 121 | LYS | C | 19.51 | 28.08 | −6.26 | 13.76 |
| 121 | LYS | O | 20.03 | 28.79 | −5.40 | 13.77 |
| 122 | VAL | N | 18.55 | 28.52 | −7.07 | 13.55 |
| 122 | VAL | CA | 18.11 | 29.90 | −6.94 | 13.26 |
| 122 | VAL | CB | 17.14 | 30.32 | −8.09 | 13.29 |
| 122 | VAL | CG1 | 17.87 | 30.24 | −9.42 | 13.16 |
| 122 | VAL | CG2 | 15.89 | 29.45 | −8.10 | 13.06 |
| 122 | VAL | C | 17.49 | 30.15 | −5.57 | 13.24 |
| 122 | VAL | O | 17.58 | 31.26 | −5.04 | 13.24 |
| 123 | CYS | N | 16.88 | 29.12 | −4.98 | 13.15 |
| 123 | CYS | CA | 16.30 | 29.26 | −3.65 | 13.17 |
| 123 | CYS | CB | 15.42 | 28.05 | −3.29 | 12.84 |
| 123 | CYS | SG | 13.89 | 27.93 | −4.26 | 12.42 |
| 123 | CYS | C | 17.40 | 29.39 | −2.60 | 13.46 |
| 123 | CYS | O | 17.29 | 30.18 | −1.68 | 13.59 |
| 124 | ASP | N | 18.45 | 28.57 | −2.72 | 13.81 |
| 124 | ASP | CA | 19.54 | 28.64 | −1.75 | 14.08 |
| 124 | ASP | CB | 20.56 | 27.51 | −1.98 | 14.29 |
| 124 | ASP | CG | 19.99 | 26.13 | −1.72 | 14.48 |
| 124 | ASP | OD1 | 18.95 | 26.02 | −1.03 | 14.63 |
| 124 | ASP | OD2 | 20.60 | 25.15 | −2.19 | 14.51 |
| 124 | ASP | C | 20.28 | 29.96 | −1.82 | 14.14 |
| 124 | ASP | O | 20.70 | 30.50 | −0.80 | 14.20 |
| 125 | VAL | N | 20.45 | 30.48 | −3.03 | 14.25 |
| 125 | VAL | CA | 21.14 | 31.75 | −3.23 | 14.33 |
| 125 | VAL | CB | 21.57 | 31.92 | −4.72 | 14.42 |
| 125 | VAL | CG1 | 22.31 | 33.25 | −4.89 | 14.41 |
| 125 | VAL | CG2 | 22.49 | 30.78 | −5.13 | 14.41 |
| 125 | VAL | C | 20.27 | 32.92 | −2.81 | 14.40 |
| 125 | VAL | O | 20.77 | 33.95 | −2.38 | 14.41 |
| 126 | ALA | N | 18.95 | 32.75 | −2.93 | 14.49 |
| 126 | ALA | CA | 18.03 | 33.80 | −2.53 | 14.56 |
| 126 | ALA | CB | 16.60 | 33.44 | −2.94 | 14.57 |
| 126 | ALA | C | 18.11 | 33.95 | −1.01 | 14.66 |
| 126 | ALA | O | 17.96 | 35.04 | −0.48 | 14.56 |
| 127 | THR | N | 18.32 | 32.83 | −0.34 | 14.79 |
| 127 | THR | CA | 18.42 | 32.83 | 1.11 | 14.94 |
| 127 | THR | CB | 18.34 | 31.41 | 1.67 | 15.05 |
| 127 | THR | OG1 | 17.03 | 30.89 | 1.44 | 15.29 |
| 127 | THR | CG2 | 18.62 | 31.42 | 3.17 | 15.24 |
| 127 | THR | C | 19.73 | 33.45 | 1.54 | 14.94 |
| 127 | THR | O | 19.80 | 34.16 | 2.54 | 14.90 |
| 128 | MET | N | 20.79 | 33.18 | 0.79 | 15.08 |
| 128 | MET | CA | 22.07 | 33.77 | 1.12 | 15.15 |
| 128 | MET | CB | 23.20 | 33.11 | 0.33 | 15.52 |
| 128 | MET | CG | 23.52 | 31.68 | 0.75 | 16.12 |
| 128 | MET | SD | 25.13 | 31.17 | 0.09 | 17.15 |
| 128 | MET | CE | 24.73 | 30.96 | −1.62 | 16.79 |
| 128 | MET | C | 22.02 | 35.26 | 0.81 | 14.99 |
| 128 | MET | O | 22.57 | 36.07 | 1.56 | 14.95 |
| 129 | ALA | N | 21.36 | 35.63 | −0.28 | 14.84 |
| 129 | ALA | CA | 21.26 | 37.04 | −0.64 | 14.81 |
| 129 | ALA | CB | 20.46 | 37.22 | −1.93 | 14.74 |
| 129 | ALA | C | 20.60 | 37.82 | 0.51 | 14.82 |
| 129 | ALA | O | 21.07 | 38.90 | 0.87 | 14.77 |
| 130 | PHE | N | 19.53 | 37.27 | 1.08 | 14.87 |
| 130 | PHE | CA | 18.84 | 37.94 | 2.17 | 14.99 |
| 130 | PHE | CB | 17.57 | 37.18 | 2.58 | 14.90 |
| 130 | PHE | CG | 16.86 | 37.80 | 3.75 | 14.91 |
| 130 | PHE | CD1 | 16.01 | 38.89 | 3.57 | 14.92 |
| 130 | PHE | CD2 | 17.12 | 37.35 | 5.03 | 14.99 |
| 130 | PHE | CE1 | 15.43 | 39.53 | 4.67 | 14.84 |
| 130 | PHE | CE2 | 16.55 | 37.98 | 6.13 | 14.95 |
| 130 | PHE | CZ | 15.70 | 39.07 | 5.95 | 14.96 |
| 130 | PHE | C | 19.72 | 38.12 | 3.41 | 15.17 |
| 130 | PHE | O | 19.74 | 39.20 | 4.02 | 15.07 |
| 131 | GLU | N | 20.45 | 37.06 | 3.78 | 15.45 |
| 131 | GLU | CA | 21.34 | 37.07 | 4.93 | 15.81 |
| 131 | GLU | CB | 21.95 | 35.68 | 5.16 | 16.12 |
| 131 | GLU | CG | 20.94 | 34.57 | 5.21 | 16.71 |
| 131 | GLU | CD | 21.57 | 33.20 | 5.39 | 16.99 |
| 131 | GLU | OE1 | 22.53 | 32.87 | 4.65 | 17.20 |
| 131 | GLU | OE2 | 21.11 | 32.46 | 6.28 | 17.36 |
| 131 | GLU | C | 22.48 | 38.07 | 4.75 | 15.87 |
| 131 | GLU | O | 22.87 | 38.75 | 5.69 | 15.81 |
| 132 | ASN | N | 23.03 | 38.14 | 3.54 | 16.02 |
| 132 | ASN | CA | 24.12 | 39.07 | 3.30 | 16.23 |
| 132 | ASN | CB | 24.77 | 38.81 | 1.95 | 16.57 |
| 132 | ASN | CG | 25.57 | 37.53 | 1.92 | 16.96 |
| 132 | ASN | OD1 | 26.25 | 37.18 | 2.89 | 17.40 |
| 132 | ASN | ND2 | 25.52 | 36.84 | 0.79 | 17.21 |
| 132 | ASN | C | 23.60 | 40.50 | 3.33 | 16.25 |
| 132 | ASN | O | 24.29 | 41.41 | 3.80 | 16.09 |
| 133 | ALA | N | 22.38 | 40.68 | 2.85 | 16.35 |
| 133 | ALA | CA | 21.74 | 41.99 | 2.81 | 16.64 |
| 133 | ALA | CB | 20.41 | 41.90 | 2.06 | 16.49 |
| 133 | ALA | C | 21.51 | 42.59 | 4.19 | 16.87 |
| 133 | ALA | O | 21.80 | 43.76 | 4.42 | 16.89 |
| 134 | ILE | N | 20.97 | 41.80 | 5.11 | 17.20 |
| 134 | ILE | CA | 20.72 | 42.32 | 6.45 | 17.59 |
| 134 | ILE | CB | 19.57 | 41.57 | 7.14 | 17.68 |
| 134 | ILE | CG2 | 18.28 | 41.77 | 6.36 | 17.80 |
| 134 | ILE | CG1 | 19.91 | 40.09 | 7.27 | 17.76 |
| 134 | ILE | CD1 | 19.02 | 39.32 | 8.24 | 17.84 |
| 134 | ILE | C | 21.95 | 42.23 | 7.34 | 17.79 |
| 134 | ILE | O | 21.91 | 42.68 | 8.49 | 17.73 |
| 135 | ALA | N | 23.03 | 41.67 | 6.81 | 18.09 |
| 135 | ALA | CA | 24.28 | 41.49 | 7.56 | 18.49 |
| 135 | ALA | CB | 25.40 | 41.08 | 6.60 | 18.54 |
| 135 | ALA | C | 24.74 | 42.68 | 8.40 | 18.75 |
| 135 | ALA | O | 25.02 | 42.52 | 9.59 | 18.80 |
| 136 | LYS | N | 24.84 | 43.85 | 7.79 | 19.02 |
| 136 | LYS | CA | 25.29 | 45.03 | 8.52 | 19.28 |
| 136 | LYS | CB | 26.62 | 45.52 | 7.93 | 19.50 |
| 136 | LYS | CG | 27.82 | 44.63 | 8.29 | 19.80 |
| 136 | LYS | CD | 29.05 | 44.98 | 7.47 | 20.05 |
| 136 | LYS | CE | 28.89 | 44.50 | 6.03 | 20.13 |
| 136 | LYS | NZ | 30.02 | 44.93 | 5.15 | 20.24 |
| 136 | LYS | C | 24.26 | 46.17 | 8.55 | 19.27 |
| 136 | LYS | O | 24.61 | 47.31 | 8.88 | 19.41 |
| 137 | VAL | N | 23.01 | 45.86 | 8.21 | 19.23 |
| 137 | VAL | CA | 21.95 | 46.86 | 8.22 | 19.12 |
| 137 | VAL | CB | 20.57 | 46.24 | 7.86 | 19.15 |
| 137 | VAL | CG1 | 19.45 | 47.23 | 8.14 | 19.26 |
| 137 | VAL | CG2 | 20.55 | 45.85 | 6.38 | 19.22 |
| 137 | VAL | C | 21.87 | 47.52 | 9.59 | 19.03 |
| 137 | VAL | O | 21.66 | 46.85 | 10.61 | 18.99 |
| 138 | LYS | N | 22.04 | 48.84 | 9.61 | 18.90 |
| 138 | LYS | CA | 22.00 | 49.63 | 10.83 | 18.76 |
| 138 | LYS | CB | 23.31 | 49.52 | 11.61 | 18.99 |
| 138 | LYS | CG | 23.27 | 48.53 | 12.76 | 19.38 |
| 138 | LYS | CD | 24.55 | 48.60 | 13.59 | 19.66 |
| 138 | LYS | CE | 24.80 | 47.28 | 14.30 | 19.83 |
| 138 | LYS | NZ | 25.05 | 46.18 | 13.32 | 20.04 |
| 138 | LYS | C | 21.77 | 51.10 | 10.53 | 18.47 |
| 138 | LYS | O | 21.96 | 51.55 | 9.41 | 18.35 |
| 139 | PRO | N | 21.36 | 51.87 | 11.55 | 18.25 |
| 139 | PRO | CD | 21.02 | 51.45 | 12.92 | 18.24 |
| 139 | PRO | CA | 21.12 | 53.29 | 11.37 | 18.17 |
| 139 | PRO | CB | 20.82 | 53.77 | 12.78 | 18.12 |
| 139 | PRO | CG | 20.15 | 52.58 | 13.39 | 18.20 |
| 139 | PRO | C | 22.36 | 53.94 | 10.77 | 18.07 |
| 139 | PRO | O | 23.48 | 53.64 | 11.20 | 18.00 |
| 140 | GLY | N | 22.15 | 54.78 | 9.77 | 17.92 |
| 140 | GLY | CA | 23.26 | 55.47 | 9.12 | 17.84 |
| 140 | GLY | C | 23.84 | 54.78 | 7.90 | 17.72 |
| 140 | GLY | O | 24.52 | 55.42 | 7.10 | 17.81 |
| 141 | THR | N | 23.57 | 53.48 | 7.73 | 17.60 |
| 141 | THR | CA | 24.11 | 52.75 | 6.59 | 17.39 |
| 141 | THR | CB | 24.09 | 51.19 | 6.83 | 17.45 |
| 141 | THR | OG1 | 22.74 | 50.70 | 6.87 | 17.43 |
| 141 | THR | CG2 | 24.78 | 50.85 | 8.15 | 17.41 |
| 141 | THR | C | 23.36 | 53.08 | 5.31 | 17.26 |
| 141 | THR | O | 22.19 | 53.44 | 5.32 | 17.27 |
| 142 | LYS | N | 24.06 | 52.96 | 4.18 | 17.06 |
| 142 | LYS | CA | 23.46 | 53.28 | 2.90 | 16.93 |
| 142 | LYS | CB | 24.54 | 53.40 | 1.83 | 17.04 |
| 142 | LYS | CG | 25.56 | 54.47 | 2.12 | 17.30 |
| 142 | LYS | CD | 26.63 | 54.50 | 1.04 | 17.61 |
| 142 | LYS | CE | 27.57 | 55.69 | 1.22 | 17.83 |
| 142 | LYS | NZ | 28.54 | 55.82 | 0.09 | 18.18 |
| 142 | LYS | C | 22.43 | 52.24 | 2.48 | 16.75 |
| 142 | LYS | O | 22.69 | 51.04 | 2.56 | 16.71 |
| 143 | LEU | N | 21.27 | 52.71 | 2.04 | 16.65 |
| 143 | LEU | CA | 20.19 | 51.83 | 1.59 | 16.50 |
| 143 | LEU | CB | 19.02 | 52.67 | 1.07 | 16.57 |
| 143 | LEU | CG | 17.82 | 51.95 | 0.44 | 16.61 |
| 143 | LEU | CD1 | 17.13 | 51.10 | 1.48 | 16.78 |
| 143 | LEU | CD2 | 16.85 | 52.99 | −0.13 | 16.73 |
| 143 | LEU | C | 20.73 | 50.95 | 0.47 | 16.39 |
| 143 | LEU | O | 20.47 | 49.75 | 0.41 | 16.43 |
| 144 | SER | N | 21.50 | 51.58 | −0.42 | 16.25 |
| 144 | SER | CA | 22.09 | 50.91 | −1.56 | 16.10 |
| 144 | SER | CB | 22.88 | 51.91 | −2.42 | 16.04 |
| 144 | SER | OG | 24.03 | 52.35 | −1.74 | 15.94 |
| 144 | SER | C | 22.98 | 49.71 | −1.22 | 16.08 |
| 144 | SER | O | 23.31 | 48.92 | −2.10 | 16.02 |
| 145 | ASN | N | 23.37 | 49.58 | 0.05 | 16.10 |
| 145 | ASN | CA | 24.21 | 48.44 | 0.45 | 16.14 |
| 145 | ASN | CB | 24.72 | 48.56 | 1.89 | 16.20 |
| 145 | ASN | CG | 25.76 | 49.64 | 2.06 | 16.36 |
| 145 | ASN | OD1 | 26.50 | 49.97 | 1.14 | 16.46 |
| 145 | ASN | ND2 | 25.84 | 50.18 | 3.28 | 16.59 |
| 145 | ASN | C | 23.45 | 47.12 | 0.34 | 16.07 |
| 145 | ASN | O | 24.05 | 46.05 | 0.15 | 15.98 |
| 146 | ILE | N | 22.13 | 47.19 | 0.48 | 16.03 |
| 146 | ILE | CA | 21.26 | 46.01 | 0.39 | 16.02 |
| 146 | ILE | CB | 19.78 | 46.39 | 0.72 | 16.04 |
| 146 | ILE | CG2 | 18.84 | 45.21 | 0.43 | 15.93 |
| 146 | ILE | CG1 | 19.67 | 46.80 | 2.19 | 16.10 |
| 146 | ILE | CD1 | 18.30 | 47.32 | 2.60 | 16.04 |
| 146 | ILE | C | 21.36 | 45.41 | −1.01 | 16.02 |
| 146 | ILE | O | 21.73 | 44.23 | −1.18 | 15.99 |
| 147 | GLY | N | 21.05 | 46.22 | −2.01 | 16.08 |
| 147 | GLY | CA | 21.11 | 45.76 | −3.39 | 16.04 |
| 147 | GLY | C | 22.53 | 45.39 | −3.77 | 16.06 |
| 147 | GLY | O | 22.76 | 44.43 | −4.50 | 16.01 |
| 148 | LYS | N | 23.50 | 46.15 | −3.26 | 16.09 |
| 148 | LYS | CA | 24.89 | 45.86 | −3.57 | 16.05 |
| 148 | LYS | CB | 25.80 | 46.88 | −2.89 | 16.28 |
| 148 | LYS | CG | 27.25 | 46.77 | −3.31 | 16.49 |
| 148 | LYS | CD | 28.10 | 47.77 | −2.53 | 16.80 |
| 148 | LYS | CE | 29.47 | 47.91 | −3.13 | 16.94 |
| 148 | LYS | NZ | 29.40 | 48.53 | −4.48 | 17.28 |
| 148 | LYS | C | 25.21 | 44.44 | −3.09 | 15.98 |
| 148 | LYS | O | 25.84 | 43.67 | −3.81 | 15.86 |
| 149 | ALA | N | 24.75 | 44.09 | −1.90 | 15.88 |
| 149 | ALA | CA | 24.99 | 42.76 | −1.34 | 15.88 |
| 149 | ALA | CB | 24.62 | 42.74 | 0.14 | 15.68 |
| 149 | ALA | C | 24.20 | 41.69 | −2.09 | 15.87 |
| 149 | ALA | O | 24.71 | 40.60 | −2.35 | 15.81 |
| 150 | VAL | N | 22.95 | 42.00 | −2.42 | 15.99 |
| 150 | VAL | CA | 22.11 | 41.06 | −3.14 | 16.15 |
| 150 | VAL | CB | 20.68 | 41.62 | −3.34 | 16.17 |
| 150 | VAL | CG1 | 19.85 | 40.67 | −4.20 | 16.18 |
| 150 | VAL | CG2 | 20.01 | 41.79 | −1.99 | 16.12 |
| 150 | VAL | C | 22.73 | 40.73 | −4.50 | 16.24 |
| 150 | VAL | O | 22.94 | 39.56 | −4.83 | 16.34 |
| 151 | HIS | N | 23.06 | 41.76 | −5.28 | 16.44 |
| 151 | HIS | CA | 23.66 | 41.54 | −6.59 | 16.71 |
| 151 | HIS | CB | 23.77 | 42.88 | −7.34 | 16.89 |
| 151 | HIS | CG | 24.27 | 42.73 | −8.75 | 17.05 |
| 151 | HIS | CD2 | 23.60 | 42.65 | −9.92 | 17.15 |
| 151 | HIS | ND1 | 25.60 | 42.60 | −9.06 | 17.14 |
| 151 | HIS | CE1 | 25.74 | 42.45 | −10.37 | 17.19 |
| 151 | HIS | NE2 | 24.53 | 42.47 | −10.91 | 17.13 |
| 151 | HIS | C | 25.02 | 40.84 | −6.53 | 16.80 |
| 151 | HIS | O | 25.38 | 40.09 | −7.43 | 16.68 |
| 152 | ASN | N | 25.77 | 41.09 | −5.46 | 16.97 |
| 152 | ASN | CA | 27.08 | 40.47 | −5.30 | 17.14 |
| 152 | ASN | CB | 27.84 | 41.13 | −4.14 | 17.36 |
| 152 | ASN | CG | 29.24 | 40.55 | −3.96 | 17.63 |
| 152 | ASN | OD1 | 29.40 | 39.46 | −3.40 | 17.87 |
| 152 | ASN | ND2 | 30.25 | 41.28 | −4.44 | 17.74 |
| 152 | ASN | C | 26.94 | 38.98 | −5.03 | 17.09 |
| 152 | ASN | O | 27.70 | 38.16 | −5.55 | 17.21 |
| 153 | THR | N | 25.95 | 38.63 | −4.21 | 17.05 |
| 153 | THR | CA | 25.71 | 37.23 | −3.89 | 16.99 |
| 153 | THR | CB | 24.62 | 37.08 | −2.82 | 16.99 |
| 153 | THR | OG1 | 25.03 | 37.76 | −1.63 | 16.88 |
| 153 | THR | CG2 | 24.40 | 35.61 | −2.49 | 16.90 |
| 153 | THR | C | 25.28 | 36.47 | −5.13 | 17.05 |
| 153 | THR | O | 25.60 | 35.30 | −5.31 | 17.02 |
| 154 | ALA | N | 24.54 | 37.16 | −6.00 | 17.09 |
| 154 | ALA | CA | 24.07 | 36.53 | −7.23 | 17.20 |
| 154 | ALA | CB | 23.04 | 37.40 | −7.91 | 17.17 |
| 154 | ALA | C | 25.25 | 36.28 | −8.16 | 17.25 |
| 154 | ALA | O | 25.36 | 35.22 | −8.77 | 17.27 |
| 155 | ARG | N | 26.14 | 37.26 | −8.25 | 17.39 |
| 155 | ARG | CA | 27.29 | 37.13 | −9.13 | 17.62 |
| 155 | ARG | CB | 28.00 | 38.47 | −9.27 | 17.94 |
| 155 | ARG | CG | 28.41 | 38.78 | −10.70 | 18.55 |
| 155 | ARG | CD | 29.51 | 37.84 | −11.16 | 19.01 |
| 155 | ARG | NE | 29.81 | 37.98 | −12.58 | 19.37 |
| 155 | ARG | CZ | 30.82 | 37.38 | −13.21 | 19.55 |
| 155 | ARG | NH1 | 31.64 | 36.58 | −12.53 | 19.64 |
| 155 | ARG | NH2 | 30.99 | 37.56 | −14.52 | 19.55 |
| 155 | ARG | C | 28.25 | 36.06 | −8.61 | 17.54 |
| 155 | ARG | O | 28.82 | 35.30 | −9.39 | 17.54 |
| 156 | GLN | N | 28.40 | 35.98 | −7.29 | 17.56 |
| 156 | GLN | CA | 29.29 | 34.98 | −6.71 | 17.53 |
| 156 | GLN | CB | 29.39 | 35.15 | −5.21 | 17.66 |
| 156 | GLN | CG | 30.15 | 36.36 | −4.72 | 18.00 |
| 156 | GLN | CD | 30.29 | 36.33 | −3.22 | 18.19 |
| 156 | GLN | OE1 | 30.55 | 37.35 | −2.58 | 18.31 |
| 156 | GLN | NE2 | 30.09 | 35.15 | −2.63 | 18.30 |
| 156 | GLN | C | 28.79 | 33.57 | −7.00 | 17.45 |
| 156 | GLN | O | 29.53 | 32.60 | −6.86 | 17.36 |
| 157 | ASN | N | 27.53 | 33.44 | −7.40 | 17.36 |
| 157 | ASN | CA | 26.98 | 32.11 | −7.70 | 17.32 |
| 157 | ASN | CB | 25.82 | 31.79 | −6.75 | 17.27 |
| 157 | ASN | CG | 26.27 | 31.65 | −5.30 | 17.29 |
| 157 | ASN | OD1 | 26.23 | 32.61 | −4.53 | 17.33 |
| 157 | ASN | ND2 | 26.73 | 30.46 | −4.93 | 17.12 |
| 157 | ASN | C | 26.54 | 31.95 | −9.14 | 17.31 |
| 157 | ASN | O | 25.69 | 31.12 | −9.46 | 17.33 |
| 158 | ASP | N | 27.14 | 32.75 | −10.03 | 17.31 |
| 158 | ASP | CA | 26.83 | 32.67 | −11.45 | 17.32 |
| 158 | ASP | CB | 27.33 | 31.33 | −12.00 | 17.60 |
| 158 | ASP | CG | 28.82 | 31.14 | −11.76 | 17.85 |
| 158 | ASP | OD1 | 29.61 | 31.93 | −12.31 | 18.05 |
| 158 | ASP | OD2 | 29.20 | 30.21 | −11.00 | 18.11 |
| 158 | ASP | C | 25.34 | 32.83 | −11.74 | 17.17 |
| 158 | ASP | O | 24.78 | 32.15 | −12.60 | 17.21 |
| 159 | LEU | N | 24.70 | 33.74 | −11.02 | 17.01 |
| 159 | LEU | CA | 23.28 | 33.99 | −11.20 | 16.85 |
| 159 | LEU | CB | 22.50 | 33.45 | −10.00 | 16.85 |
| 159 | LEU | CG | 22.57 | 31.93 | −9.80 | 16.82 |
| 159 | LEU | CD1 | 21.84 | 31.53 | −8.53 | 16.77 |
| 159 | LEU | CD2 | 21.95 | 31.24 | −11.01 | 16.80 |
| 159 | LEU | C | 23.00 | 35.47 | −11.39 | 16.84 |
| 159 | LEU | O | 23.91 | 36.30 | −11.32 | 16.75 |
| 160 | LYS | N | 21.74 | 35.80 | −11.64 | 16.81 |
| 160 | LYS | CA | 21.33 | 37.18 | −11.86 | 16.76 |
| 160 | LYS | CB | 20.80 | 37.33 | −13.29 | 16.93 |
| 160 | LYS | CG | 21.85 | 37.21 | −14.39 | 17.27 |
| 160 | LYS | CD | 22.92 | 38.28 | −14.24 | 17.53 |
| 160 | LYS | CE | 23.76 | 38.43 | −15.52 | 17.74 |
| 160 | LYS | NZ | 24.38 | 37.14 | −15.94 | 17.92 |
| 160 | LYS | C | 20.24 | 37.60 | −10.88 | 16.64 |
| 160 | LYS | O | 19.78 | 36.81 | −10.07 | 16.58 |
| 161 | VAL | N | 19.85 | 38.87 | −10.94 | 16.56 |
| 161 | VAL | CA | 18.80 | 39.39 | −10.07 | 16.48 |
| 161 | VAL | CB | 19.36 | 40.47 | −9.11 | 16.50 |
| 161 | VAL | CG1 | 20.54 | 39.90 | −8.30 | 16.47 |
| 161 | VAL | CG2 | 19.82 | 41.69 | −9.91 | 16.49 |
| 161 | VAL | C | 17.71 | 40.02 | −10.93 | 16.42 |
| 161 | VAL | O | 17.95 | 40.34 | −12.09 | 16.44 |
| 162 | ILE | N | 16.50 | 40.18 | −10.38 | 16.31 |
| 162 | ILE | CA | 15.43 | 40.82 | −11.14 | 16.20 |
| 162 | ILE | CB | 14.03 | 40.33 | −10.70 | 16.18 |
| 162 | ILE | CG2 | 12.94 | 41.12 | −11.43 | 16.13 |
| 162 | ILE | CG1 | 13.89 | 38.83 | −10.97 | 16.16 |
| 162 | ILE | CD1 | 14.17 | 38.42 | −12.40 | 16.13 |
| 162 | ILE | C | 15.57 | 42.30 | −10.81 | 16.12 |
| 162 | ILE | O | 15.30 | 42.72 | −9.69 | 16.22 |
| 163 | LYS | N | 16.00 | 43.09 | −11.79 | 16.08 |
| 163 | LYS | CA | 16.22 | 44.52 | −11.58 | 16.01 |
| 163 | LYS | CB | 17.15 | 45.06 | −12.68 | 16.13 |
| 163 | LYS | CG | 18.55 | 44.49 | −12.61 | 16.32 |
| 163 | LYS | CD | 19.43 | 45.05 | −13.72 | 16.46 |
| 163 | LYS | CE | 20.87 | 44.61 | −13.57 | 16.66 |
| 163 | LYS | NZ | 21.43 | 45.01 | −12.23 | 16.68 |
| 163 | LYS | C | 14.97 | 45.38 | −11.49 | 15.89 |
| 163 | LYS | O | 14.96 | 46.40 | −10.80 | 15.78 |
| 164 | ASN | N | 13.91 | 44.98 | −12.19 | 15.71 |
| 164 | ASN | CA | 12.69 | 45.77 | −12.18 | 15.65 |
| 164 | ASN | CB | 12.06 | 45.76 | −13.58 | 15.73 |
| 164 | ASN | CG | 11.60 | 44.39 | −13.99 | 15.84 |
| 164 | ASN | OD1 | 12.15 | 43.38 | −13.56 | 15.98 |
| 164 | ASN | ND2 | 10.59 | 44.34 | −14.86 | 16.02 |
| 164 | ASN | C | 11.66 | 45.34 | −11.12 | 15.55 |
| 164 | ASN | O | 10.47 | 45.56 | −11.28 | 15.53 |
| 165 | LEU | N | 12.14 | 44.71 | −10.05 | 15.46 |
| 165 | LEU | CA | 11.28 | 44.32 | −8.93 | 15.40 |
| 165 | LEU | CB | 10.99 | 42.81 | −8.92 | 15.50 |
| 165 | LEU | CG | 9.91 | 42.36 | −9.92 | 15.52 |
| 165 | LEU | CD1 | 9.65 | 40.86 | −9.79 | 15.40 |
| 165 | LEU | CD2 | 8.64 | 43.13 | −9.66 | 15.58 |
| 165 | LEU | C | 12.07 | 44.74 | −7.69 | 15.32 |
| 165 | LEU | O | 13.24 | 44.40 | −7.56 | 15.29 |
| 166 | THR | N | 11.44 | 45.48 | −6.79 | 15.23 |
| 166 | THR | CA | 12.15 | 45.97 | −5.62 | 15.22 |
| 166 | THR | CB | 12.45 | 47.46 | −5.76 | 15.24 |
| 166 | THR | OG1 | 11.27 | 48.20 | −5.42 | 15.38 |
| 166 | THR | CG2 | 12.83 | 47.80 | −7.19 | 15.22 |
| 166 | THR | C | 11.42 | 45.84 | −4.30 | 15.16 |
| 166 | THR | O | 10.21 | 45.60 | −4.26 | 15.11 |
| 167 | GLY | N | 12.19 | 46.03 | −3.23 | 15.10 |
| 167 | GLY | CA | 11.65 | 46.00 | −1.89 | 14.99 |
| 167 | GLY | C | 11.04 | 47.36 | −1.64 | 15.01 |
| 167 | GLY | O | 10.97 | 48.19 | −2.56 | 14.96 |
| 168 | HIS | N | 10.61 | 47.62 | −0.41 | 14.93 |
| 168 | HIS | CA | 9.95 | 48.88 | −0.11 | 14.90 |
| 168 | HIS | CB | 8.60 | 48.90 | −0.82 | 14.90 |
| 168 | HIS | CG | 7.79 | 47.65 | −0.62 | 14.94 |
| 168 | HIS | CD2 | 7.63 | 46.56 | −1.40 | 14.92 |
| 168 | HIS | ND1 | 7.07 | 47.41 | 0.53 | 15.02 |
| 168 | HIS | CE1 | 6.50 | 46.22 | 0.45 | 14.97 |
| 168 | HIS | NE2 | 6.82 | 45.69 | −0.71 | 15.02 |
| 168 | HIS | C | 9.72 | 49.06 | 1.38 | 14.86 |
| 168 | HIS | O | 9.73 | 48.09 | 2.14 | 14.84 |
| 169 | GLY | N | 9.51 | 50.30 | 1.79 | 14.86 |
| 169 | GLY | CA | 9.23 | 50.56 | 3.19 | 14.88 |
| 169 | GLY | C | 7.86 | 49.98 | 3.43 | 14.92 |
| 169 | GLY | O | 7.09 | 49.80 | 2.48 | 14.96 |
| 170 | VAL | N | 7.54 | 49.69 | 4.68 | 14.98 |
| 170 | VAL | CA | 6.24 | 49.12 | 5.03 | 15.11 |
| 170 | VAL | CB | 6.23 | 47.57 | 4.83 | 15.20 |
| 170 | VAL | CG1 | 7.23 | 46.92 | 5.76 | 15.19 |
| 170 | VAL | CG2 | 4.82 | 47.01 | 5.05 | 15.26 |
| 170 | VAL | C | 5.94 | 49.49 | 6.48 | 15.15 |
| 170 | VAL | O | 6.85 | 49.88 | 7.22 | 15.12 |
| 171 | GLY | N | 4.68 | 49.42 | 6.88 | 15.28 |
| 171 | GLY | CA | 4.33 | 49.77 | 8.24 | 15.58 |
| 171 | GLY | C | 2.88 | 50.18 | 8.39 | 15.74 |
| 171 | GLY | O | 2.09 | 49.44 | 8.96 | 15.67 |
| 172 | LEU | N | 2.54 | 51.36 | 7.88 | 16.08 |
| 172 | LEU | CA | 1.17 | 51.87 | 7.94 | 16.42 |
| 172 | LEU | CB | 1.19 | 53.37 | 8.29 | 16.64 |
| 172 | LEU | CG | 1.71 | 53.71 | 9.69 | 16.88 |
| 172 | LEU | CD1 | 1.96 | 55.21 | 9.82 | 17.03 |
| 172 | LEU | CD2 | 0.70 | 53.23 | 10.72 | 16.95 |
| 172 | LEU | C | 0.47 | 51.66 | 6.60 | 16.59 |
| 172 | LEU | O | −0.69 | 52.03 | 6.41 | 16.58 |
| 173 | SER | N | 1.19 | 51.04 | 5.67 | 16.80 |
| 173 | SER | CA | 0.69 | 50.75 | 4.33 | 17.10 |
| 173 | SER | CB | 0.71 | 52.01 | 3.46 | 17.01 |
| 173 | SER | OG | 2.04 | 52.31 | 3.06 | 17.12 |
| 173 | SER | C | 1.64 | 49.71 | 3.74 | 17.32 |
| 173 | SER | O | 2.72 | 49.48 | 4.28 | 17.43 |
| 174 | LEU | N | 1.26 | 49.11 | 2.62 | 17.59 |
| 174 | LEU | CA | 2.10 | 48.09 | 2.00 | 17.97 |
| 174 | LEU | CB | 1.30 | 47.30 | 0.96 | 18.07 |
| 174 | LEU | CG | 1.97 | 46.09 | 0.32 | 18.14 |
| 174 | LEU | CD1 | 2.47 | 45.13 | 1.39 | 18.13 |
| 174 | LEU | CD2 | 0.95 | 45.39 | −0.59 | 18.21 |
| 174 | LEU | C | 3.34 | 48.71 | 1.34 | 18.15 |
| 174 | LEU | O | 4.42 | 48.12 | 1.36 | 18.23 |
| 175 | HIS | N | 3.18 | 49.90 | 0.76 | 18.25 |
| 175 | HIS | CA | 4.28 | 50.58 | 0.08 | 18.42 |
| 175 | HIS | CB | 4.04 | 50.59 | −1.44 | 18.61 |
| 175 | HIS | CG | 4.17 | 49.23 | −2.08 | 18.85 |
| 175 | HIS | CD2 | 4.94 | 48.81 | −3.11 | 18.95 |
| 175 | HIS | ND1 | 3.45 | 48.13 | −1.67 | 18.99 |
| 175 | HIS | CE1 | 3.77 | 47.09 | −2.41 | 18.96 |
| 175 | HIS | NE2 | 4.68 | 47.47 | −3.30 | 19.08 |
| 175 | HIS | C | 4.49 | 52.03 | 0.56 | 18.34 |
| 175 | HIS | O | 3.65 | 52.90 | 0.33 | 18.40 |
| 176 | GLU | N | 5.61 | 52.28 | 1.24 | 18.24 |
| 176 | GLU | CA | 5.93 | 53.63 | 1.70 | 18.03 |
| 176 | GLU | CB | 5.33 | 53.88 | 3.10 | 18.11 |
| 176 | GLU | CG | 5.67 | 52.86 | 4.18 | 18.06 |
| 176 | GLU | CD | 4.71 | 52.91 | 5.37 | 18.15 |
| 176 | GLU | OE1 | 5.17 | 52.70 | 6.51 | 18.07 |
| 176 | GLU | OE2 | 3.50 | 53.15 | 5.16 | 18.21 |
| 176 | GLU | C | 7.45 | 53.86 | 1.68 | 17.99 |
| 176 | GLU | O | 8.22 | 52.96 | 1.34 | 17.91 |
| 177 | ALA | N | 7.88 | 55.08 | 2.01 | 17.92 |
| 177 | ALA | CA | 9.31 | 55.41 | 1.99 | 17.96 |
| 177 | ALA | CB | 9.53 | 56.84 | 2.48 | 17.88 |
| 177 | ALA | C | 10.12 | 54.44 | 2.83 | 17.98 |
| 177 | ALA | O | 9.72 | 54.08 | 3.94 | 17.95 |
| 178 | PRO | N | 11.28 | 53.97 | 2.31 | 18.09 |
| 178 | PRO | CD | 12.20 | 53.09 | 3.05 | 18.06 |
| 178 | PRO | CA | 11.82 | 54.30 | 0.99 | 18.05 |
| 178 | PRO | CB | 13.26 | 53.80 | 1.08 | 18.09 |
| 178 | PRO | CG | 13.13 | 52.60 | 1.96 | 18.15 |
| 178 | PRO | C | 11.02 | 53.62 | −0.12 | 18.12 |
| 178 | PRO | O | 10.65 | 52.45 | 0.00 | 18.11 |
| 179 | ALA | N | 10.75 | 54.36 | −1.18 | 18.08 |
| 179 | ALA | CA | 9.97 | 53.86 | −2.31 | 18.04 |
| 179 | ALA | CB | 9.95 | 54.91 | −3.43 | 18.07 |
| 179 | ALA | C | 10.45 | 52.53 | −2.86 | 18.02 |
| 179 | ALA | O | 9.67 | 51.59 | −3.01 | 17.95 |
| 180 | HIS | N | 11.74 | 52.45 | −3.16 | 18.03 |
| 180 | HIS | CA | 12.31 | 51.23 | −3.72 | 18.10 |
| 180 | HIS | CB | 12.59 | 51.42 | −5.21 | 18.27 |
| 180 | HIS | CG | 11.38 | 51.76 | −6.02 | 18.41 |
| 180 | HIS | CD2 | 10.39 | 50.97 | −6.50 | 18.41 |
| 180 | HIS | ND1 | 11.04 | 53.05 | −6.36 | 18.52 |
| 180 | HIS | CE1 | 9.90 | 53.04 | −7.03 | 18.46 |
| 180 | HIS | NE2 | 9.48 | 51.79 | −7.12 | 18.51 |
| 180 | HIS | C | 13.59 | 50.74 | −3.05 | 18.07 |
| 180 | HIS | O | 14.48 | 51.51 | −2.70 | 18.04 |
| 181 | VAL | N | 13.66 | 49.43 | −2.87 | 17.99 |
| 181 | VAL | CA | 14.82 | 48.78 | −2.29 | 17.99 |
| 181 | VAL | CB | 14.43 | 47.92 | −1.09 | 17.93 |
| 181 | VAL | CG1 | 15.68 | 47.23 | −0.54 | 17.99 |
| 181 | VAL | CG2 | 13.75 | 48.77 | −0.02 | 17.87 |
| 181 | VAL | C | 15.32 | 47.89 | −3.41 | 18.04 |
| 181 | VAL | O | 14.89 | 46.74 | −3.54 | 17.87 |
| 182 | LEU | N | 16.23 | 48.42 | −4.23 | 18.15 |
| 182 | LEU | CA | 16.76 | 47.70 | −5.38 | 18.28 |
| 182 | LEU | CB | 17.66 | 48.63 | −6.22 | 18.30 |
| 182 | LEU | CG | 17.08 | 49.95 | −6.73 | 18.37 |
| 182 | LEU | CD1 | 18.18 | 50.75 | −7.44 | 18.37 |
| 182 | LEU | CD2 | 15.92 | 49.68 | −7.67 | 18.45 |
| 182 | LEU | C | 17.55 | 46.45 | −5.04 | 18.41 |
| 182 | LEU | O | 18.11 | 46.32 | −3.95 | 18.36 |
| 183 | ASN | N | 17.61 | 45.54 | −6.00 | 18.53 |
| 183 | ASN | CA | 18.35 | 44.30 | −5.83 | 18.76 |
| 183 | ASN | CB | 17.60 | 43.14 | −6.46 | 18.76 |
| 183 | ASN | CG | 16.38 | 42.75 | −5.66 | 18.80 |
| 183 | ASN | OD1 | 16.46 | 42.59 | −4.44 | 18.80 |
| 183 | ASN | ND2 | 15.24 | 42.60 | −6.33 | 18.88 |
| 183 | ASN | C | 19.75 | 44.42 | −6.43 | 18.90 |
| 183 | ASN | O | 20.41 | 43.42 | −6.69 | 18.79 |
| 184 | TYR | N | 20.18 | 45.66 | −6.66 | 19.15 |
| 184 | TYR | CA | 21.51 | 45.92 | −7.20 | 19.45 |
| 184 | TYR | CB | 21.52 | 45.90 | −8.73 | 19.31 |
| 184 | TYR | CG | 20.61 | 46.93 | −9.37 | 19.35 |
| 184 | TYR | CD1 | 19.23 | 46.74 | −9.41 | 19.28 |
| 184 | TYR | CE1 | 18.39 | 47.70 | −9.96 | 19.27 |
| 184 | TYR | CD2 | 21.14 | 48.11 | −9.91 | 19.32 |
| 184 | TYR | CE2 | 20.30 | 49.08 | −10.46 | 19.31 |
| 184 | TYR | CZ | 18.93 | 48.87 | −10.48 | 19.30 |
| 184 | TYR | OH | 18.10 | 49.84 | −10.99 | 19.38 |
| 184 | TYR | C | 21.99 | 47.27 | −6.69 | 19.73 |
| 184 | TYR | O | 21.22 | 48.03 | −6.11 | 19.62 |
| 185 | PHE | N | 23.27 | 47.55 | −6.91 | 20.14 |
| 185 | PHE | CA | 23.89 | 48.78 | −6.45 | 20.57 |
| 185 | PHE | CB | 25.41 | 48.57 | −6.30 | 20.66 |
| 185 | PHE | CG | 26.15 | 49.81 | −5.86 | 20.88 |
| 185 | PHE | CD1 | 25.84 | 50.43 | −4.66 | 20.87 |
| 185 | PHE | CD2 | 27.15 | 50.36 | −6.66 | 20.96 |
| 185 | PHE | CE1 | 26.50 | 51.59 | −4.25 | 20.95 |
| 185 | PHE | CE2 | 27.83 | 51.52 | −6.26 | 21.07 |
| 185 | PHE | CZ | 27.50 | 52.13 | −5.05 | 20.97 |
| 185 | PHE | C | 23.64 | 50.01 | −7.33 | 20.87 |
| 185 | PHE | O | 23.79 | 49.97 | −8.55 | 20.82 |
| 186 | ASP | N | 23.26 | 51.11 | −6.68 | 21.25 |
| 186 | ASP | CA | 23.00 | 52.37 | −7.37 | 21.64 |
| 186 | ASP | CB | 21.52 | 52.73 | −7.28 | 21.78 |
| 186 | ASP | CG | 21.21 | 54.09 | −7.89 | 22.06 |
| 186 | ASP | OD1 | 21.88 | 54.47 | −8.88 | 22.17 |
| 186 | ASP | OD2 | 20.30 | 54.77 | −7.38 | 22.18 |
| 186 | ASP | C | 23.86 | 53.44 | −6.72 | 21.81 |
| 186 | ASP | O | 23.54 | 53.97 | −5.66 | 21.77 |
| 187 | PRO | N | 24.99 | 53.78 | −7.36 | 22.04 |
| 187 | PRO | CD | 25.36 | 53.33 | −8.71 | 22.02 |
| 187 | PRO | CA | 25.94 | 54.79 | −6.88 | 22.12 |
| 187 | PRO | CB | 27.03 | 54.78 | −7.95 | 22.13 |
| 187 | PRO | CG | 26.26 | 54.46 | −9.19 | 22.06 |
| 187 | PRO | C | 25.39 | 56.19 | −6.63 | 22.29 |
| 187 | PRO | O | 25.97 | 56.96 | −5.87 | 22.43 |
| 188 | LYS | N | 24.28 | 56.53 | −7.27 | 22.39 |
| 188 | LYS | CA | 23.74 | 57.88 | −7.08 | 22.50 |
| 188 | LYS | CB | 22.83 | 58.25 | −8.25 | 22.67 |
| 188 | LYS | CG | 21.56 | 57.43 | −8.31 | 22.90 |
| 188 | LYS | CD | 20.43 | 58.19 | −9.00 | 23.09 |
| 188 | LYS | CE | 19.08 | 57.62 | −8.59 | 23.18 |
| 188 | LYS | NZ | 18.87 | 57.74 | −7.11 | 23.28 |
| 188 | LYS | C | 22.95 | 58.02 | −5.78 | 22.48 |
| 188 | LYS | O | 22.93 | 59.09 | −5.18 | 22.53 |
| 189 | ASP | N | 22.31 | 56.94 | −5.36 | 22.45 |
| 189 | ASP | CA | 21.50 | 56.96 | −4.15 | 22.28 |
| 189 | ASP | CB | 20.72 | 55.65 | −4.05 | 22.42 |
| 189 | ASP | CG | 19.71 | 55.65 | −2.92 | 22.58 |
| 189 | ASP | OD1 | 19.08 | 56.71 | −2.69 | 22.65 |
| 189 | ASP | OD2 | 19.54 | 54.59 | −2.29 | 22.55 |
| 189 | ASP | C | 22.28 | 57.22 | −2.87 | 22.11 |
| 189 | ASP | O | 23.25 | 56.52 | −2.56 | 22.13 |
| 190 | LYS | N | 21.85 | 58.23 | −2.12 | 21.87 |
| 190 | LYS | CA | 22.48 | 58.60 | −0.86 | 21.58 |
| 190 | LYS | CB | 22.88 | 60.08 | −0.89 | 21.79 |
| 190 | LYS | CG | 24.05 | 60.40 | −1.83 | 22.07 |
| 190 | LYS | CD | 24.19 | 61.89 | −2.06 | 22.28 |
| 190 | LYS | CE | 25.42 | 62.21 | −2.90 | 22.41 |
| 190 | LYS | NZ | 25.46 | 63.65 | −3.30 | 22.59 |
| 190 | LYS | C | 21.54 | 58.33 | 0.30 | 21.22 |
| 190 | LYS | O | 21.83 | 58.69 | 1.45 | 21.28 |
| 191 | THR | N | 20.40 | 57.72 | 0.01 | 20.83 |
| 191 | THR | CA | 19.42 | 57.40 | 1.04 | 20.38 |
| 191 | THR | CB | 18.25 | 56.60 | 0.47 | 20.44 |
| 191 | THR | OG1 | 17.64 | 57.35 | −0.60 | 20.58 |
| 191 | THR | CG2 | 17.21 | 56.33 | 1.56 | 20.48 |
| 191 | THR | C | 20.04 | 56.56 | 2.14 | 19.97 |
| 191 | THR | O | 20.74 | 55.59 | 1.87 | 19.84 |
| 192 | LEU | N | 19.78 | 56.95 | 3.38 | 19.60 |
| 192 | LEU | CA | 20.33 | 56.21 | 4.51 | 19.23 |
| 192 | LEU | CB | 21.14 | 57.15 | 5.41 | 19.30 |
| 192 | LEU | CG | 22.37 | 57.83 | 4.79 | 19.35 |
| 192 | LEU | CD1 | 23.02 | 58.72 | 5.84 | 19.39 |
| 192 | LEU | CD2 | 23.34 | 56.78 | 4.30 | 19.38 |
| 192 | LEU | C | 19.23 | 55.57 | 5.33 | 18.95 |
| 192 | LEU | O | 18.07 | 56.00 | 5.29 | 18.85 |
| 193 | LEU | N | 19.59 | 54.52 | 6.06 | 18.63 |
| 193 | LEU | CA | 18.65 | 53.83 | 6.92 | 18.35 |
| 193 | LEU | CB | 19.08 | 52.37 | 7.13 | 18.28 |
| 193 | LEU | CG | 19.36 | 51.55 | 5.87 | 18.21 |
| 193 | LEU | CD1 | 19.73 | 50.12 | 6.27 | 18.19 |
| 193 | LEU | CD2 | 18.14 | 51.53 | 4.97 | 18.19 |
| 193 | LEU | C | 18.66 | 54.56 | 8.26 | 18.22 |
| 193 | LEU | O | 19.71 | 55.01 | 8.72 | 18.20 |
| 194 | THR | N | 17.50 | 54.67 | 8.90 | 18.00 |
| 194 | THR | CA | 17.44 | 55.35 | 10.18 | 17.86 |
| 194 | THR | CB | 16.54 | 56.60 | 10.09 | 17.92 |
| 194 | THR | OG1 | 15.17 | 56.20 | 9.93 | 18.08 |
| 194 | THR | CG2 | 16.94 | 57.46 | 8.89 | 17.92 |
| 194 | THR | C | 16.92 | 54.43 | 11.27 | 17.66 |
| 194 | THR | O | 16.32 | 53.39 | 10.99 | 17.58 |
| 195 | GLU | N | 17.18 | 54.80 | 12.52 | 17.44 |
| 195 | GLU | CA | 16.71 | 54.01 | 13.66 | 17.18 |
| 195 | GLU | CB | 17.17 | 54.66 | 14.97 | 17.32 |
| 195 | GLU | CG | 16.60 | 54.04 | 16.25 | 17.58 |
| 195 | GLU | CD | 17.12 | 52.64 | 16.54 | 17.71 |
| 195 | GLU | OE1 | 18.36 | 52.43 | 16.54 | 17.75 |
| 195 | GLU | OE2 | 16.28 | 51.74 | 16.79 | 17.91 |
| 195 | GLU | C | 15.19 | 53.92 | 13.60 | 16.93 |
| 195 | GLU | O | 14.50 | 54.93 | 13.48 | 16.84 |
| 196 | GLY | N | 14.67 | 52.70 | 13.67 | 16.63 |
| 196 | GLY | CA | 13.23 | 52.50 | 13.65 | 16.30 |
| 196 | GLY | C | 12.63 | 52.33 | 12.26 | 16.10 |
| 196 | GLY | O | 11.43 | 52.07 | 12.13 | 16.02 |
| 197 | MET | N | 13.45 | 52.47 | 11.23 | 15.88 |
| 197 | MET | CA | 12.96 | 52.33 | 9.86 | 15.62 |
| 197 | MET | CB | 14.06 | 52.72 | 8.86 | 15.66 |
| 197 | MET | CG | 13.61 | 52.66 | 7.42 | 15.69 |
| 197 | MET | SD | 14.88 | 53.15 | 6.23 | 15.97 |
| 197 | MET | CE | 14.71 | 54.91 | 6.26 | 15.86 |
| 197 | MET | C | 12.52 | 50.89 | 9.60 | 15.51 |
| 197 | MET | O | 13.24 | 49.94 | 9.93 | 15.43 |
| 198 | VAL | N | 11.33 | 50.74 | 9.03 | 15.40 |
| 198 | VAL | CA | 10.80 | 49.42 | 8.72 | 15.29 |
| 198 | VAL | CB | 9.36 | 49.23 | 9.23 | 15.28 |
| 198 | VAL | CG1 | 8.92 | 47.78 | 8.99 | 15.33 |
| 198 | VAL | CG2 | 9.29 | 49.57 | 10.70 | 15.26 |
| 198 | VAL | C | 10.82 | 49.22 | 7.22 | 15.23 |
| 198 | VAL | O | 10.24 | 50.00 | 6.46 | 15.07 |
| 199 | LEU | N | 11.48 | 48.15 | 6.81 | 15.15 |
| 199 | LEU | CA | 11.64 | 47.83 | 5.41 | 15.10 |
| 199 | LEU | CB | 13.12 | 47.97 | 5.03 | 15.18 |
| 199 | LEU | CG | 13.85 | 49.25 | 5.42 | 15.43 |
| 199 | LEU | CD1 | 15.34 | 49.08 | 5.18 | 15.50 |
| 199 | LEU | CD2 | 13.30 | 50.40 | 4.59 | 15.42 |
| 199 | LEU | C | 11.22 | 46.41 | 5.06 | 15.00 |
| 199 | LEU | O | 11.28 | 45.51 | 5.90 | 14.97 |
| 200 | ALA | N | 10.80 | 46.23 | 3.81 | 14.88 |
| 200 | ALA | CA | 10.44 | 44.92 | 3.29 | 14.79 |
| 200 | ALA | CB | 9.13 | 44.99 | 2.49 | 14.85 |
| 200 | ALA | C | 11.62 | 44.63 | 2.36 | 14.74 |
| 200 | ALA | O | 11.75 | 45.27 | 1.32 | 14.57 |
| 201 | ILE | N | 12.49 | 43.70 | 2.76 | 14.69 |
| 201 | ILE | CA | 13.65 | 43.34 | 1.95 | 14.73 |
| 201 | ILE | CB | 14.91 | 43.17 | 2.84 | 14.73 |
| 201 | ILE | CG2 | 16.14 | 42.87 | 1.98 | 14.64 |
| 201 | ILE | CG1 | 15.14 | 44.47 | 3.63 | 14.74 |
| 201 | ILE | CD1 | 16.27 | 44.40 | 4.64 | 14.87 |
| 201 | ILE | C | 13.31 | 42.03 | 1.26 | 14.86 |
| 201 | ILE | O | 13.02 | 41.03 | 1.91 | 14.72 |
| 202 | GLU | N | 13.31 | 42.05 | −0.07 | 15.03 |
| 202 | GLU | CA | 12.93 | 40.89 | −0.86 | 15.23 |
| 202 | GLU | CB | 11.44 | 40.97 | −1.21 | 15.70 |
| 202 | GLU | CG | 10.96 | 42.35 | −1.62 | 16.37 |
| 202 | GLU | CD | 9.49 | 42.36 | −2.03 | 16.74 |
| 202 | GLU | OE1 | 8.92 | 43.45 | −2.23 | 17.06 |
| 202 | GLU | OE2 | 8.90 | 41.26 | −2.16 | 17.24 |
| 202 | GLU | C | 13.75 | 40.64 | −2.12 | 15.14 |
| 202 | GLU | O | 13.30 | 40.92 | −3.24 | 15.18 |
| 203 | PRO | N | 14.95 | 40.08 | −1.97 | 15.00 |
| 203 | PRO | CD | 15.57 | 39.65 | −0.70 | 14.95 |
| 203 | PRO | CA | 15.83 | 39.79 | −3.10 | 14.80 |
| 203 | PRO | CB | 17.13 | 39.37 | −2.43 | 14.93 |
| 203 | PRO | CG | 16.67 | 38.72 | −1.17 | 14.97 |
| 203 | PRO | C | 15.26 | 38.72 | −4.02 | 14.86 |
| 203 | PRO | O | 14.69 | 37.73 | −3.56 | 14.73 |
| 204 | PHE | N | 15.39 | 38.95 | −5.32 | 14.81 |
| 204 | PHE | CA | 14.94 | 38.03 | −6.35 | 14.77 |
| 204 | PHE | CB | 14.10 | 38.73 | −7.42 | 14.87 |
| 204 | PHE | CG | 12.70 | 39.01 | −7.00 | 15.14 |
| 204 | PHE | CD1 | 12.37 | 40.22 | −6.40 | 15.15 |
| 204 | PHE | CD2 | 11.70 | 38.08 | −7.25 | 15.18 |
| 204 | PHE | CE1 | 11.05 | 40.50 | −6.06 | 15.35 |
| 204 | PHE | CE2 | 10.39 | 38.34 | −6.92 | 15.30 |
| 204 | PHE | CZ | 10.06 | 39.56 | −6.32 | 15.32 |
| 204 | PHE | C | 16.19 | 37.51 | −7.04 | 14.65 |
| 204 | PHE | O | 17.00 | 38.30 | −7.50 | 14.68 |
| 205 | ILE | N | 16.34 | 36.19 | −7.10 | 14.51 |
| 205 | ILE | CA | 17.49 | 35.59 | −7.75 | 14.35 |
| 205 | ILE | CB | 18.22 | 34.62 | −6.82 | 14.37 |
| 205 | ILE | CG2 | 19.36 | 33.92 | −7.57 | 14.34 |
| 205 | ILE | CG1 | 18.77 | 35.38 | −5.61 | 14.42 |
| 205 | ILE | CD1 | 19.82 | 36.43 | −5.97 | 14.39 |
| 205 | ILE | C | 16.97 | 34.84 | −8.98 | 14.25 |
| 205 | ILE | O | 16.04 | 34.04 | −8.87 | 14.18 |
| 206 | SER | N | 17.59 | 35.11 | −10.12 | 14.20 |
| 206 | SER | CA | 17.19 | 34.52 | −11.39 | 14.18 |
| 206 | SER | CB | 16.78 | 35.62 | −12.36 | 14.18 |
| 206 | SER | OG | 16.50 | 35.09 | −13.65 | 13.98 |
| 206 | SER | C | 18.25 | 33.65 | −12.07 | 14.23 |
| 206 | SER | O | 19.44 | 33.97 | −12.04 | 14.08 |
| 207 | SER | N | 17.80 | 32.58 | −12.71 | 14.35 |
| 207 | SER | CA | 18.71 | 31.69 | −13.42 | 14.55 |
| 207 | SER | CB | 18.02 | 30.36 | −13.74 | 14.39 |
| 207 | SER | OG | 16.86 | 30.58 | −14.52 | 14.75 |
| 207 | SER | C | 19.25 | 32.33 | −14.70 | 14.70 |
| 207 | SER | O | 20.26 | 31.89 | −15.24 | 14.74 |
| 208 | ASN | N | 18.57 | 33.36 | −15.20 | 14.97 |
| 208 | ASN | CA | 19.03 | 34.02 | −16.42 | 15.29 |
| 208 | ASN | CB | 18.68 | 33.17 | −17.64 | 15.62 |
| 208 | ASN | CG | 19.16 | 33.80 | −18.95 | 15.88 |
| 208 | ASN | OD1 | 18.43 | 34.58 | −19.58 | 16.25 |
| 208 | ASN | ND2 | 20.38 | 33.47 | −19.36 | 16.23 |
| 208 | ASN | C | 18.48 | 35.42 | −16.61 | 15.36 |
| 208 | ASN | O | 19.24 | 36.40 | −16.61 | 15.34 |
| 209 | ALA | N | 17.16 | 35.52 | −16.78 | 15.46 |
| 209 | ALA | CA | 16.51 | 36.81 | −16.99 | 15.60 |
| 209 | ALA | CB | 15.01 | 36.62 | −17.11 | 15.50 |
| 209 | ALA | C | 16.83 | 37.80 | −15.87 | 15.74 |
| 209 | ALA | O | 16.81 | 37.44 | −14.69 | 15.71 |
| 210 | SER | N | 17.09 | 39.04 | −16.25 | 15.95 |
| 210 | SER | CA | 17.41 | 40.08 | −15.27 | 16.16 |
| 210 | SER | CB | 18.63 | 40.88 | −15.73 | 16.25 |
| 210 | SER | OG | 18.37 | 41.56 | −16.95 | 16.30 |
| 210 | SER | C | 16.23 | 41.02 | −15.05 | 16.31 |
| 210 | SER | O | 16.36 | 42.06 | −14.40 | 16.36 |
| 211 | PHE | N | 15.08 | 40.66 | −15.60 | 16.42 |
| 211 | PHE | CA | 13.86 | 41.45 | −15.45 | 16.55 |
| 211 | PHE | CB | 13.84 | 42.61 | −16.44 | 16.50 |
| 211 | PHE | CG | 13.81 | 42.17 | −17.87 | 16.55 |
| 211 | PHE | CD1 | 14.98 | 41.86 | −18.54 | 16.55 |
| 211 | PHE | CD2 | 12.60 | 42.04 | −18.54 | 16.55 |
| 211 | PHE | CE1 | 14.96 | 41.42 | −19.85 | 16.56 |
| 211 | PHE | CE2 | 12.57 | 41.59 | −19.86 | 16.56 |
| 211 | PHE | CZ | 13.75 | 41.28 | −20.52 | 16.52 |
| 211 | PHE | C | 12.62 | 40.59 | −15.70 | 16.67 |
| 211 | PHE | O | 12.73 | 39.45 | −16.16 | 16.65 |
| 212 | VAL | N | 11.46 | 41.15 | −15.38 | 16.75 |
| 212 | VAL | CA | 10.19 | 40.46 | −15.59 | 16.95 |
| 212 | VAL | CB | 9.43 | 40.21 | −14.25 | 17.00 |
| 212 | VAL | CG1 | 10.28 | 39.38 | −13.31 | 17.04 |
| 212 | VAL | CG2 | 9.05 | 41.52 | −13.60 | 17.07 |
| 212 | VAL | C | 9.28 | 41.28 | −16.50 | 17.20 |
| 212 | VAL | O | 9.52 | 42.47 | −16.73 | 17.16 |
| 213 | THR | N | 8.24 | 40.64 | −17.02 | 17.39 |
| 213 | THR | CA | 7.28 | 41.31 | −17.89 | 17.65 |
| 213 | THR | CB | 7.56 | 41.01 | −19.38 | 17.64 |
| 213 | THR | OG1 | 7.64 | 39.59 | −19.58 | 17.57 |
| 213 | THR | CG2 | 8.85 | 41.66 | −19.83 | 17.66 |
| 213 | THR | C | 5.87 | 40.88 | −17.54 | 18.01 |
| 213 | THR | O | 5.66 | 39.94 | −16.77 | 17.88 |
| 214 | GLU | N | 4.89 | 41.57 | −18.11 | 18.45 |
| 214 | GLU | CA | 3.49 | 41.25 | −17.86 | 18.82 |
| 214 | GLU | CB | 2.59 | 42.16 | −18.69 | 19.08 |
| 214 | GLU | CG | 1.10 | 41.98 | −18.43 | 19.59 |
| 214 | GLU | CD | 0.27 | 43.06 | −19.09 | 19.87 |
| 214 | GLU | OE1 | 0.40 | 44.24 | −18.68 | 20.15 |
| 214 | GLU | OE2 | −0.50 | 42.73 | −20.02 | 19.93 |
| 214 | GLU | C | 3.26 | 39.80 | −18.23 | 18.94 |
| 214 | GLU | O | 3.73 | 39.33 | −19.27 | 18.97 |
| 215 | GLY | N | 2.56 | 39.08 | −17.37 | 19.16 |
| 215 | GLY | CA | 2.29 | 37.67 | −17.61 | 19.41 |
| 215 | GLY | C | 0.97 | 37.35 | −18.30 | 19.60 |
| 215 | GLY | O | 0.42 | 38.19 | −19.01 | 19.60 |
| 216 | LYS | N | 0.48 | 36.14 | −18.06 | 19.72 |
| 216 | LYS | CA | −0.77 | 35.64 | −18.64 | 19.90 |
| 216 | LYS | CB | −0.99 | 34.18 | −18.24 | 19.92 |
| 216 | LYS | CG | −1.17 | 33.95 | −16.73 | 19.88 |
| 216 | LYS | CD | −1.38 | 32.46 | −16.39 | 19.88 |
| 216 | LYS | CE | −0.16 | 31.60 | −16.71 | 19.86 |
| 216 | LYS | NZ | 1.05 | 31.94 | −15.91 | 19.72 |
| 216 | LYS | C | −2.00 | 36.46 | −18.26 | 20.01 |
| 216 | LYS | O | −3.10 | 36.18 | −18.74 | 20.08 |
| 217 | ASN | N | −1.82 | 37.45 | −17.39 | 20.11 |
| 217 | ASN | CA | −2.92 | 38.31 | −16.97 | 20.17 |
| 217 | ASN | CB | −3.73 | 37.64 | −15.85 | 20.15 |
| 217 | ASN | CG | −2.87 | 37.27 | −14.64 | 20.08 |
| 217 | ASN | OD1 | −2.25 | 38.12 | −14.02 | 20.11 |
| 217 | ASN | ND2 | −2.84 | 35.98 | −14.32 | 19.98 |
| 217 | ASN | C | −2.42 | 39.67 | −16.52 | 20.25 |
| 217 | ASN | O | −1.23 | 39.96 | −16.55 | 20.34 |
| 218 | GLU | N | −3.35 | 40.53 | −16.11 | 20.42 |
| 218 | GLU | CA | −3.00 | 41.88 | −15.68 | 20.46 |
| 218 | GLU | CB | −4.26 | 42.73 | −15.68 | 20.71 |
| 218 | GLU | CG | −5.29 | 42.24 | −14.67 | 21.21 |
| 218 | GLU | CD | −6.70 | 42.34 | −15.17 | 21.50 |
| 218 | GLU | OE1 | −7.06 | 41.59 | −16.11 | 21.77 |
| 218 | GLU | OE2 | −7.46 | 43.17 | −14.63 | 21.76 |
| 218 | GLU | C | −2.36 | 41.94 | −14.29 | 20.26 |
| 218 | GLU | O | −2.17 | 43.02 | −13.75 | 20.26 |
| 219 | TRP | N | −2.03 | 40.79 | −13.71 | 20.11 |
| 219 | TRP | CA | −1.41 | 40.80 | −12.39 | 19.87 |
| 219 | TRP | CB | −2.32 | 40.13 | −11.36 | 20.05 |
| 219 | TRP | CG | −3.62 | 40.85 | −11.11 | 20.27 |
| 219 | TRP | CD2 | −3.81 | 42.02 | −10.31 | 20.38 |
| 219 | TRP | CE2 | −5.20 | 42.32 | −10.34 | 20.46 |
| 219 | TRP | CE3 | −2.96 | 42.85 | −9.57 | 20.47 |
| 219 | TRP | CD1 | −4.85 | 40.50 | −11.58 | 20.32 |
| 219 | TRP | NE1 | −5.80 | 41.37 | −11.12 | 20.35 |
| 219 | TRP | CZ2 | −5.75 | 43.41 | −9.66 | 20.48 |
| 219 | TRP | CZ3 | −3.51 | 43.93 | −8.89 | 20.51 |
| 219 | TRP | CH2 | −4.89 | 44.20 | −8.94 | 20.54 |
| 219 | TRP | C | −0.04 | 40.15 | −12.35 | 19.69 |
| 219 | TRP | O | 0.96 | 40.81 | −12.12 | 19.59 |
| 220 | ALA | N | 0.00 | 38.84 | −12.59 | 19.51 |
| 220 | ALA | CA | 1.24 | 38.08 | −12.54 | 19.25 |
| 220 | ALA | CB | 0.97 | 36.63 | −12.92 | 19.25 |
| 220 | ALA | C | 2.40 | 38.61 | −13.37 | 19.12 |
| 220 | ALA | O | 2.22 | 39.17 | −14.45 | 19.08 |
| 221 | PHE | N | 3.60 | 38.42 | −12.84 | 18.89 |
| 221 | PHE | CA | 4.83 | 38.82 | −13.51 | 18.74 |
| 221 | PHE | CB | 5.70 | 39.63 | −12.55 | 18.94 |
| 221 | PHE | CG | 5.31 | 41.08 | −12.46 | 19.00 |
| 221 | PHE | CD1 | 5.68 | 41.98 | −13.46 | 19.09 |
| 221 | PHE | CD2 | 4.58 | 41.56 | −11.38 | 19.11 |
| 221 | PHE | CE1 | 5.34 | 43.33 | −13.38 | 19.14 |
| 221 | PHE | CE2 | 4.23 | 42.92 | −11.29 | 19.18 |
| 221 | PHE | CZ | 4.62 | 43.80 | −12.29 | 19.15 |
| 221 | PHE | C | 5.53 | 37.55 | −13.94 | 18.55 |
| 221 | PHE | O | 5.52 | 36.55 | −13.23 | 18.59 |
| 222 | GLU | N | 6.15 | 37.59 | −15.11 | 18.27 |
| 222 | GLU | CA | 6.83 | 36.41 | −15.62 | 18.12 |
| 222 | GLU | CB | 5.86 | 35.62 | −16.51 | 18.26 |
| 222 | GLU | CG | 4.63 | 35.09 | −15.80 | 18.52 |
| 222 | GLU | CD | 3.64 | 34.48 | −16.77 | 18.69 |
| 222 | GLU | OE1 | 4.10 | 33.85 | −17.75 | 18.85 |
| 222 | GLU | OE2 | 2.41 | 34.62 | −16.56 | 18.82 |
| 222 | GLU | C | 8.07 | 36.75 | −16.43 | 17.90 |
| 222 | GLU | O | 8.44 | 37.91 | −16.57 | 17.79 |
| 223 | THR | N | 8.72 | 35.71 | −16.94 | 17.70 |
| 223 | THR | CA | 9.89 | 35.90 | −17.80 | 17.51 |
| 223 | THR | CB | 11.22 | 35.43 | −17.14 | 17.48 |
| 223 | THR | OG1 | 11.19 | 34.02 | −16.91 | 17.44 |
| 223 | THR | CG2 | 11.45 | 36.15 | −15.82 | 17.47 |
| 223 | THR | C | 9.60 | 35.08 | −19.04 | 17.44 |
| 223 | THR | O | 9.18 | 33.92 | −18.96 | 17.45 |
| 224 | SER | N | 9.79 | 35.69 | −20.21 | 17.33 |
| 224 | SER | CA | 9.53 | 34.99 | −21.46 | 17.23 |
| 224 | SER | CB | 9.71 | 35.95 | −22.64 | 17.38 |
| 224 | SER | OG | 8.62 | 36.86 | −22.70 | 17.48 |
| 224 | SER | C | 10.41 | 33.76 | −21.63 | 17.10 |
| 224 | SER | O | 9.97 | 32.75 | −22.17 | 17.02 |
| 225 | ASP | N | 11.65 | 33.83 | −21.15 | 16.90 |
| 225 | ASP | CA | 12.55 | 32.68 | −21.28 | 16.73 |
| 225 | ASP | CB | 14.02 | 33.14 | −21.29 | 16.73 |
| 225 | ASP | CG | 14.46 | 33.75 | −19.96 | 16.81 |
| 225 | ASP | OD1 | 13.65 | 33.85 | −19.02 | 16.77 |
| 225 | ASP | OD2 | 15.66 | 34.11 | −19.87 | 16.89 |
| 225 | ASP | C | 12.33 | 31.64 | −20.18 | 16.56 |
| 225 | ASP | O | 13.05 | 30.64 | −20.10 | 16.51 |
| 226 | LYS | N | 11.31 | 31.87 | −19.36 | 16.40 |
| 226 | LYS | CA | 10.94 | 30.98 | −18.26 | 16.33 |
| 226 | LYS | CB | 10.36 | 29.67 | −18.79 | 16.51 |
| 226 | LYS | CG | 9.05 | 29.85 | −19.56 | 16.77 |
| 226 | LYS | CD | 8.47 | 28.50 | −19.99 | 17.17 |
| 226 | LYS | CE | 7.14 | 28.68 | −20.71 | 17.26 |
| 226 | LYS | NZ | 7.27 | 29.60 | −21.87 | 17.54 |
| 226 | LYS | C | 12.09 | 30.68 | −17.30 | 16.20 |
| 226 | LYS | O | 12.28 | 29.54 | −16.87 | 16.10 |
| 227 | SER | N | 12.87 | 31.71 | −16.96 | 16.08 |
| 227 | SER | CA | 13.97 | 31.57 | −16.02 | 15.95 |
| 227 | SER | CB | 14.70 | 32.91 | −15.81 | 16.03 |
| 227 | SER | OG | 15.51 | 33.29 | −16.91 | 16.31 |
| 227 | SER | C | 13.41 | 31.12 | −14.68 | 15.82 |
| 227 | SER | O | 12.24 | 31.38 | −14.37 | 15.82 |
| 228 | PHE | N | 14.22 | 30.45 | −13.88 | 15.63 |
| 228 | PHE | CA | 13.79 | 30.03 | −12.55 | 15.45 |
| 228 | PHE | CB | 14.47 | 28.70 | −12.16 | 15.62 |
| 228 | PHE | CG | 14.11 | 27.55 | −13.07 | 15.67 |
| 228 | PHE | CD1 | 12.79 | 27.34 | −13.44 | 15.84 |
| 228 | PHE | CD2 | 15.10 | 26.71 | −13.57 | 15.80 |
| 228 | PHE | CE1 | 12.44 | 26.30 | −14.31 | 15.82 |
| 228 | PHE | CE2 | 14.77 | 25.66 | −14.44 | 15.84 |
| 228 | PHE | CZ | 13.43 | 25.46 | −14.81 | 15.83 |
| 228 | PHE | C | 14.18 | 31.15 | −11.60 | 15.24 |
| 228 | PHE | O | 15.36 | 31.46 | −11.44 | 15.22 |
| 229 | VAL | N | 13.19 | 31.79 | −10.99 | 15.08 |
| 229 | VAL | CA | 13.44 | 32.90 | −10.08 | 14.85 |
| 229 | VAL | CB | 12.71 | 34.18 | −10.56 | 14.85 |
| 229 | VAL | CG1 | 13.06 | 35.34 | −9.65 | 14.82 |
| 229 | VAL | CG2 | 13.10 | 34.49 | −12.00 | 14.91 |
| 229 | VAL | C | 12.95 | 32.56 | −8.68 | 14.68 |
| 229 | VAL | O | 11.87 | 32.00 | −8.51 | 14.62 |
| 230 | ALA | N | 13.75 | 32.88 | −7.67 | 14.53 |
| 230 | ALA | CA | 13.36 | 32.60 | −6.30 | 14.46 |
| 230 | ALA | CB | 14.28 | 31.55 | −5.67 | 14.43 |
| 230 | ALA | C | 13.43 | 33.89 | −5.49 | 14.45 |
| 230 | ALA | O | 14.26 | 34.76 | −5.74 | 14.30 |
| 231 | GLN | N | 12.54 | 34.00 | −4.51 | 14.42 |
| 231 | GLN | CA | 12.52 | 35.18 | −3.68 | 14.46 |
| 231 | GLN | CB | 11.35 | 36.08 | −4.09 | 14.81 |
| 231 | GLN | CG | 11.08 | 37.22 | −3.12 | 15.42 |
| 231 | GLN | CD | 9.93 | 38.12 | −3.57 | 15.89 |
| 231 | GLN | OE1 | 8.88 | 37.65 | −4.01 | 16.22 |
| 231 | GLN | NE2 | 10.14 | 39.44 | −3.45 | 16.19 |
| 231 | GLN | C | 12.40 | 34.81 | −2.21 | 14.32 |
| 231 | GLN | O | 11.82 | 33.77 | −1.85 | 14.22 |
| 232 | ILE | N | 12.98 | 35.64 | −1.35 | 14.25 |
| 232 | ILE | CA | 12.89 | 35.45 | 0.09 | 14.16 |
| 232 | ILE | CB | 14.23 | 35.00 | 0.72 | 14.31 |
| 232 | ILE | CG2 | 14.13 | 35.10 | 2.24 | 14.35 |
| 232 | ILE | CG1 | 14.54 | 33.56 | 0.32 | 14.40 |
| 232 | ILE | CD1 | 13.45 | 32.55 | 0.68 | 14.48 |
| 232 | ILE | C | 12.53 | 36.85 | 0.54 | 14.10 |
| 232 | ILE | O | 13.18 | 37.81 | 0.14 | 14.07 |
| 233 | GLU | N | 11.48 | 36.99 | 1.34 | 14.08 |
| 233 | GLU | CA | 11.07 | 38.32 | 1.81 | 14.04 |
| 233 | GLU | CB | 9.84 | 38.79 | 1.01 | 14.33 |
| 233 | GLU | CG | 9.27 | 40.14 | 1.47 | 14.79 |
| 233 | GLU | CD | 8.21 | 40.71 | 0.51 | 15.04 |
| 233 | GLU | OE1 | 7.36 | 39.94 | 0.02 | 15.11 |
| 233 | GLU | OE2 | 8.24 | 41.95 | 0.26 | 15.46 |
| 233 | GLU | C | 10.77 | 38.36 | 3.31 | 13.88 |
| 233 | GLU | O | 10.19 | 37.44 | 3.87 | 13.77 |
| 234 | HIS | N | 11.20 | 39.45 | 3.95 | 13.74 |
| 234 | HIS | CA | 10.97 | 39.65 | 5.38 | 13.62 |
| 234 | HIS | CB | 12.17 | 39.17 | 6.22 | 13.64 |
| 234 | HIS | CG | 12.16 | 37.70 | 6.52 | 13.64 |
| 234 | HIS | CD2 | 12.97 | 36.70 | 6.10 | 13.71 |
| 234 | HIS | ND1 | 11.23 | 37.12 | 7.35 | 13.58 |
| 234 | HIS | CE1 | 11.47 | 35.82 | 7.43 | 13.70 |
| 234 | HIS | NE2 | 12.52 | 35.54 | 6.68 | 13.68 |
| 234 | HIS | C | 10.77 | 41.13 | 5.66 | 13.69 |
| 234 | HIS | O | 11.28 | 41.99 | 4.94 | 13.41 |
| 235 | THR | N | 10.00 | 41.41 | 6.70 | 13.74 |
| 235 | THR | CA | 9.77 | 42.77 | 7.12 | 14.10 |
| 235 | THR | CB | 8.34 | 42.95 | 7.67 | 13.95 |
| 235 | THR | OG1 | 7.41 | 42.77 | 6.59 | 14.05 |
| 235 | THR | CG2 | 8.14 | 44.34 | 8.25 | 14.01 |
| 235 | THR | C | 10.80 | 42.95 | 8.23 | 14.34 |
| 235 | THR | O | 10.81 | 42.19 | 9.21 | 14.21 |
| 236 | VAL | N | 11.67 | 43.94 | 8.06 | 14.72 |
| 236 | VAL | CA | 12.75 | 44.20 | 9.00 | 15.16 |
| 236 | VAL | CB | 14.13 | 44.04 | 8.30 | 15.24 |
| 236 | VAL | CG1 | 15.25 | 44.23 | 9.30 | 15.36 |
| 236 | VAL | CG2 | 14.23 | 42.68 | 7.63 | 15.41 |
| 236 | VAL | C | 12.72 | 45.61 | 9.58 | 15.38 |
| 236 | VAL | O | 12.45 | 46.57 | 8.87 | 15.38 |
| 237 | ILE | N | 13.01 | 45.73 | 10.87 | 15.72 |
| 237 | ILE | CA | 13.06 | 47.02 | 11.53 | 16.09 |
| 237 | ILE | CB | 12.30 | 47.01 | 12.86 | 16.19 |
| 237 | ILE | CG2 | 12.44 | 48.38 | 13.54 | 16.19 |
| 237 | ILE | CG1 | 10.83 | 46.67 | 12.64 | 16.14 |
| 237 | ILE | CD1 | 10.00 | 46.61 | 13.92 | 16.23 |
| 237 | ILE | C | 14.54 | 47.28 | 11.81 | 16.43 |
| 237 | ILE | O | 15.22 | 46.44 | 12.40 | 16.46 |
| 238 | VAL | N | 15.03 | 48.44 | 11.38 | 16.76 |
| 238 | VAL | CA | 16.42 | 48.81 | 11.59 | 17.25 |
| 238 | VAL | CB | 16.86 | 49.87 | 10.54 | 17.25 |
| 238 | VAL | CG1 | 18.34 | 50.19 | 10.69 | 17.33 |
| 238 | VAL | CG2 | 16.55 | 49.35 | 9.14 | 17.35 |
| 238 | VAL | C | 16.58 | 49.38 | 13.00 | 17.55 |
| 238 | VAL | O | 15.87 | 50.32 | 13.37 | 17.65 |
| 239 | THR | N | 17.47 | 48.80 | 13.80 | 17.94 |
| 239 | THR | CA | 17.70 | 49.28 | 15.16 | 18.35 |
| 239 | THR | CB | 17.01 | 48.40 | 16.23 | 18.40 |
| 239 | THR | OG1 | 17.94 | 47.42 | 16.70 | 18.56 |
| 239 | THR | CG2 | 15.79 | 47.70 | 15.64 | 18.48 |
| 239 | THR | C | 19.19 | 49.29 | 15.46 | 18.59 |
| 239 | THR | O | 19.97 | 48.59 | 14.82 | 18.59 |
| 240 | LYS | N | 19.58 | 50.09 | 16.46 | 18.88 |
| 240 | LYS | CA | 20.98 | 50.19 | 16.84 | 19.14 |
| 240 | LYS | CB | 21.13 | 51.17 | 18.01 | 19.18 |
| 240 | LYS | CG | 20.26 | 50.84 | 19.20 | 19.31 |
| 240 | LYS | CD | 20.32 | 51.95 | 20.24 | 19.42 |
| 240 | LYS | CE | 19.32 | 51.70 | 21.35 | 19.54 |
| 240 | LYS | NZ | 17.94 | 51.52 | 20.78 | 19.63 |
| 240 | LYS | C | 21.58 | 48.85 | 17.23 | 19.31 |
| 240 | LYS | O | 22.76 | 48.59 | 16.99 | 19.41 |
| 241 | ASP | N | 20.76 | 47.99 | 17.82 | 19.56 |
| 241 | ASP | CA | 21.23 | 46.68 | 18.26 | 19.74 |
| 241 | ASP | CB | 20.45 | 46.25 | 19.50 | 19.96 |
| 241 | ASP | CG | 20.56 | 47.25 | 20.62 | 20.19 |
| 241 | ASP | OD1 | 21.70 | 47.51 | 21.06 | 20.32 |
| 241 | ASP | OD2 | 19.51 | 47.77 | 21.06 | 20.24 |
| 241 | ASP | C | 21.13 | 45.61 | 17.20 | 19.71 |
| 241 | ASP | O | 21.30 | 44.42 | 17.49 | 19.76 |
| 242 | GLY | N | 20.85 | 46.01 | 15.96 | 19.64 |
| 242 | GLY | CA | 20.72 | 45.05 | 14.89 | 19.44 |
| 242 | GLY | C | 19.32 | 44.98 | 14.31 | 19.27 |
| 242 | GLY | O | 18.37 | 45.49 | 14.91 | 19.42 |
| 243 | PRO | N | 19.15 | 44.36 | 13.13 | 19.10 |
| 243 | PRO | CD | 20.19 | 43.72 | 12.30 | 19.09 |
| 243 | PRO | CA | 17.84 | 44.25 | 12.49 | 18.93 |
| 243 | PRO | CB | 18.19 | 43.76 | 11.08 | 18.96 |
| 243 | PRO | CG | 19.37 | 42.91 | 11.32 | 18.97 |
| 243 | PRO | C | 16.86 | 43.32 | 13.20 | 18.84 |
| 243 | PRO | O | 17.24 | 42.26 | 13.71 | 18.83 |
| 244 | ILE | N | 15.60 | 43.72 | 13.22 | 18.63 |
| 244 | ILE | CA | 14.56 | 42.94 | 13.85 | 18.44 |
| 244 | ILE | CB | 13.77 | 43.77 | 14.87 | 18.58 |
| 244 | ILE | CG2 | 12.60 | 42.95 | 15.40 | 18.60 |
| 244 | ILE | CG1 | 14.69 | 44.22 | 16.00 | 18.60 |
| 244 | ILE | CD1 | 14.01 | 45.12 | 17.01 | 18.87 |
| 244 | ILE | C | 13.61 | 42.46 | 12.77 | 18.24 |
| 244 | ILE | O | 12.97 | 43.27 | 12.10 | 18.19 |
| 245 | LEU | N | 13.53 | 41.15 | 12.60 | 18.04 |
| 245 | LEU | CA | 12.64 | 40.57 | 11.60 | 17.82 |
| 245 | LEU | CB | 13.26 | 39.30 | 11.01 | 17.85 |
| 245 | LEU | CG | 14.34 | 39.49 | 9.95 | 17.94 |
| 245 | LEU | CD1 | 15.49 | 40.33 | 10.50 | 17.98 |
| 245 | LEU | CD2 | 14.82 | 38.13 | 9.50 | 17.98 |
| 245 | LEU | C | 11.30 | 40.23 | 12.25 | 17.67 |
| 245 | LEU | O | 11.21 | 39.28 | 13.02 | 17.72 |
| 246 | THR | N | 10.27 | 40.99 | 11.93 | 17.42 |
| 246 | THR | CA | 8.96 | 40.73 | 12.50 | 17.16 |
| 246 | THR | CB | 8.01 | 41.92 | 12.28 | 17.11 |
| 246 | THR | OG1 | 7.78 | 42.08 | 10.88 | 16.92 |
| 246 | THR | CG2 | 8.61 | 43.21 | 12.84 | 17.02 |
| 246 | THR | C | 8.31 | 39.49 | 11.87 | 17.16 |
| 246 | THR | O | 7.41 | 38.90 | 12.46 | 17.01 |
| 247 | THR | N | 8.79 | 39.10 | 10.69 | 17.17 |
| 247 | THR | CA | 8.23 | 37.97 | 9.96 | 17.28 |
| 247 | THR | CB | 8.10 | 38.30 | 8.47 | 17.17 |
| 247 | THR | OG1 | 9.38 | 38.69 | 7.96 | 17.06 |
| 247 | THR | CG2 | 7.10 | 39.43 | 8.27 | 17.15 |
| 247 | THR | C | 9.02 | 36.67 | 10.07 | 17.60 |
| 247 | THR | O | 8.81 | 35.72 | 9.31 | 17.40 |
| 248 | LYS | N | 9.95 | 36.63 | 11.02 | 18.00 |
| 248 | LYS | CA | 10.76 | 35.45 | 11.26 | 18.41 |
| 248 | LYS | CB | 12.25 | 35.81 | 11.21 | 18.46 |
| 248 | LYS | CG | 13.18 | 34.62 | 11.42 | 18.68 |
| 248 | LYS | CD | 14.29 | 34.98 | 12.41 | 18.86 |
| 248 | LYS | CE | 15.29 | 33.85 | 12.59 | 18.87 |
| 248 | LYS | NZ | 16.27 | 33.83 | 11.46 | 18.92 |
| 248 | LYS | C | 10.41 | 34.95 | 12.65 | 18.63 |
| 248 | LYS | O | 10.35 | 35.72 | 13.61 | 18.70 |
| 249 | ILE | N | 10.15 | 33.65 | 12.73 | 18.88 |
| 249 | ILE | CA | 9.82 | 32.99 | 13.98 | 19.17 |
| 249 | ILE | CB | 8.69 | 31.97 | 13.79 | 19.27 |
| 249 | ILE | CG2 | 8.35 | 31.30 | 15.12 | 19.37 |
| 249 | ILE | CG1 | 7.47 | 32.68 | 13.20 | 19.19 |
| 249 | ILE | CD1 | 6.26 | 31.78 | 13.00 | 19.11 |
| 249 | ILE | C | 11.07 | 32.24 | 14.44 | 19.30 |
| 249 | ILE | O | 11.17 | 31.05 | 14.12 | 19.48 |
| 249 | ILE | OT | 11.94 | 32.88 | 15.08 | 19.56 |
| 301 | HOH | O | 15.58 | 45.78 | −8.07 | 13.29 |
| 302 | HOH | O | 8.20 | 30.81 | −9.11 | 9.94 |
| 303 | HOH | O | 27.39 | 35.54 | −0.53 | 14.83 |
| 304 | HOH | O | 5.50 | 43.91 | 10.59 | 9.76 |
| 305 | HOH | O | 10.59 | 34.42 | 4.24 | 7.93 |
| 306 | HOH | O | 19.45 | 47.41 | 12.36 | 15.72 |
| 307 | HOH | O | −1.29 | 38.04 | 1.88 | 14.01 |
| 308 | HOH | O | 13.24 | 24.25 | −1.59 | 14.18 |
| 309 | HOH | O | −13.22 | 32.82 | −2.07 | 15.50 |
| 310 | HOH | O | 18.12 | 39.61 | −19.01 | 14.85 |
| 311 | HOH | O | 0.03 | 23.25 | 6.53 | 17.05 |
| 312 | HOH | O | 1.50 | 24.00 | −11.29 | 15.56 |
| 313 | HOH | O | −9.35 | 32.09 | −0.58 | 13.40 |
| 314 | HOH | O | 10.19 | 52.79 | 6.21 | 17.63 |
| 315 | HOH | O | −8.01 | 22.26 | −10.70 | 14.85 |
| 316 | HOH | O | −2.36 | 51.98 | 8.35 | 14.01 |
| 317 | HOH | O | 6.27 | 15.67 | −3.49 | 17.58 |
| 318 | HOH | O | 10.53 | 30.89 | −11.09 | 17.26 |
| 319 | HOH | O | 10.13 | 38.60 | −20.14 | 12.45 |
| 320 | HOH | O | 30.28 | 34.23 | −0.09 | 17.92 |
| 321 | HOH | O | −0.84 | 44.52 | 14.44 | 16.58 |
| 322 | HOH | O | 26.21 | 57.81 | 7.05 | 13.30 |
| 323 | HOH | O | 19.17 | 48.50 | −2.42 | 17.17 |
| 324 | HOH | O | 13.11 | 36.66 | −20.68 | 15.09 |
| 325 | HOH | O | 6.96 | 38.85 | 14.99 | 15.43 |
| 326 | HOH | O | 9.75 | 31.91 | 10.28 | 14.40 |
| 327 | HOH | O | 4.86 | 28.36 | 12.29 | 19.91 |
| 328 | HOH | O | 21.80 | 54.71 | −0.54 | 16.23 |
| 329 | HOH | O | 6.66 | 25.26 | 6.89 | 14.98 |
| 330 | HOH | O | 24.86 | 54.37 | −3.29 | 21.95 |
| 331 | HOH | O | 7.98 | 55.20 | 5.75 | 15.42 |
| 332 | HOH | O | −3.38 | 22.91 | −13.66 | 19.49 |
| 333 | HOH | O | 10.11 | 34.59 | −7.28 | 17.80 |
| 334 | HOH | O | 9.10 | 26.82 | −16.09 | 17.48 |
| 335 | HOH | O | 3.05 | 15.74 | −9.90 | 15.64 |
| 336 | HOH | O | 6.54 | 57.38 | 3.22 | 19.58 |
| 337 | HOH | O | 4.19 | 48.03 | 26.45 | 17.54 |
| 338 | HOH | O | 6.37 | 21.07 | −0.89 | 16.77 |
| 339 | HOH | O | 2.25 | 19.30 | 6.94 | 19.90 |
| 340 | HOH | O | 10.04 | 28.93 | −15.26 | 15.12 |
| 341 | HOH | O | 0.00 | 48.16 | 19.92 | 16.41 |
| 342 | HOH | O | 26.78 | 44.53 | −6.33 | 17.16 |
| 343 | HOH | O | −5.06 | 33.66 | −15.77 | 16.23 |
| 344 | HOH | O | 13.65 | 54.53 | −3.06 | 19.30 |
| 345 | HOH | O | −17.57 | 28.43 | 10.03 | 18.58 |
| 346 | HOH | O | 3.93 | 35.26 | −5.28 | 17.46 |
| 347 | HOH | O | −8.81 | 29.06 | −11.84 | 17.53 |
| 348 | HOH | O | −1.49 | 34.38 | −12.13 | 16.89 |
| 349 | HOH | O | −12.43 | 26.89 | −3.00 | 15.32 |
| 350 | HOH | O | 10.54 | 30.05 | 6.06 | 16.74 |
| 351 | HOH | O | 1.05 | 28.19 | −16.99 | 18.50 |
| 352 | H0H | O | 21.27 | 28.86 | 1.27 | 19.15 |
| 353 | HOH | O | 11.88 | 20.15 | −2.30 | 18.43 |
| 354 | HOH | O | 10.52 | 47.72 | −15.77 | 19.17 |
| 355 | HOH | O | 22.27 | 40.37 | −12.00 | 16.97 |
| 356 | HOH | O | 10.42 | 12.95 | −7.41 | 19.92 |
| 357 | HOH | O | 9.19 | 23.21 | 1.40 | 13.30 |
| 358 | HOH | O | 6.64 | 26.05 | 16.30 | 22.39 |
| 359 | HOH | O | −9.67 | 25.19 | −6.10 | 21.60 |
| 360 | HOH | O | 14.70 | 25.83 | −0.04 | 19.19 |
| 361 | HOH | O | −5.83 | 29.97 | −21.37 | 21.00 |
| 362 | HOH | O | 17.48 | 51.03 | −2.79 | 18.87 |
| 363 | HOH | O | 17.34 | 25.55 | −18.63 | 19.45 |
| 364 | HOH | O | 21.00 | 22.98 | −6.51 | 16.39 |
| 365 | HOH | O | 19.18 | 57.03 | 12.95 | 17.28 |
| 366 | HOH | O | 24.93 | 34.77 | 7.98 | 20.06 |
| 367 | HOH | O | 29.01 | 42.16 | −7.78 | 21.98 |
| 368 | HOH | O | 7.30 | 21.19 | −12.40 | 20.66 |
| 369 | HOH | O | 1.85 | 39.52 | −1.07 | 21.23 |
| 370 | HOH | O | −11.25 | 37.95 | 12.27 | 18.94 |
| 371 | HOH | O | 5.68 | 17.09 | −12.51 | 14.63 |
| 372 | HOH | O | 18.83 | 59.66 | 4.15 | 19.42 |
| 373 | HOH | O | −2.45 | 39.18 | −20.31 | 20.20 |
| 374 | HOH | O | −2.89 | 51.67 | 4.45 | 18.29 |
| 375 | HOH | O | −3.65 | 18.15 | −1.24 | 20.93 |
| 376 | HOH | O | −7.38 | 24.80 | 2.09 | 20.51 |
| 377 | HOH | O | −1.20 | 30.35 | −12.83 | 16.70 |
| 378 | HOH | O | −11.24 | 29.00 | 3.59 | 17.09 |
| 379 | HOH | O | 11.02 | 20.77 | −13.07 | 20.24 |
| 380 | HOH | O | 20.19 | 60.08 | −2.96 | 22.38 |
| 381 | HOH | O | −5.47 | 56.35 | 23.01 | 20.13 |
| 382 | HOH | O | 14.16 | 56.73 | −10.30 | 23.05 |
| 383 | HOH | O | 5.84 | 30.08 | 17.45 | 23.80 |
| 384 | HOH | O | −1.10 | 26.40 | 11.23 | 18.74 |
| 385 | HOH | O | 13.21 | 53.06 | 17.30 | 20.35 |
| 386 | HOH | O | 26.15 | 36.60 | −12.84 | 18.40 |
| 387 | HOH | O | 26.31 | 44.66 | 4.11 | 22.33 |
| 388 | HOH | O | 7.42 | 53.55 | 24.05 | 20.26 |
| 389 | HOH | O | −15.40 | 25.50 | −5.02 | 21.75 |
| 390 | HOH | O | −9.94 | 49.33 | 0.99 | 21.69 |
| 391 | HOH | O | −9.82 | 30.09 | −18.49 | 22.08 |
| 392 | HOH | O | 8.73 | 17.60 | −15.37 | 22.36 |
| 393 | HOH | O | 14.56 | 39.48 | 14.70 | 18.48 |
| 394 | HOH | O | 33.14 | 35.08 | −14.69 | 21.34 |
| 395 | HOH | O | −11.29 | 30.98 | 10.83 | 21.35 |
| 396 | HOH | O | 6.51 | 57.12 | 9.90 | 21.88 |
| 397 | HOH | O | 4.94 | 40.34 | −21.52 | 21.03 |
| 398 | HOH | O | 18.05 | 58.88 | −4.38 | 22.13 |
| 399 | HOH | O | 22.46 | 37.96 | 8.51 | 16.98 |
| 400 | HOH | O | −6.56 | 43.63 | 18.04 | 20.38 |
| 401 | HOH | O | 6.20 | 21.84 | 9.02 | 22.53 |
| 402 | HOH | O | 11.54 | 60.10 | 2.44 | 19.72 |
| 403 | HOH | O | 10.16 | 58.97 | −13.02 | 20.98 |
| 404 | HOH | O | 20.30 | 28.25 | 5.09 | 22.10 |
| 405 | HOH | O | −8.58 | 38.95 | 4.27 | 15.56 |
| 406 | HOH | O | 15.79 | 36.64 | −21.40 | 24.14 |
| 407 | HOH | O | −3.69 | 16.22 | 2.31 | 20.43 |
| 408 | HOH | O | 11.80 | 57.04 | 5.08 | 17.94 |
| 409 | HOH | O | 20.26 | 54.34 | 17.28 | 22.77 |
| 410 | HOH | O | 28.85 | 64.98 | −2.73 | 20.92 |
| 411 | HOH | O | 24.31 | 29.52 | −12.44 | 19.06 |
| 412 | HOH | O | 9.29 | 13.36 | −4.52 | 19.92 |
| 413 | HOH | O | −6.69 | 47.05 | 9.87 | 22.55 |
| 414 | HOH | O | 27.01 | 45.74 | 0.80 | 12.75 |
| 415 | HOH | O | −1.12 | 19.80 | 8.30 | 19.44 |
| 416 | HOH | O | 30.00 | 56.11 | −14.13 | 25.81 |
| 417 | HOH | O | 12.09 | 32.76 | 8.32 | 18.82 |
| 418 | HOH | O | −14.13 | 30.16 | −15.63 | 23.51 |
| 419 | HOH | O | 8.46 | 45.06 | −17.68 | 20.53 |
| 420 | HOH | O | −8.04 | 37.23 | 17.14 | 22.73 |
| 421 | HOH | O | 0.05 | 58.45 | 19.88 | 22.71 |
| 422 | HOH | O | 10.70 | 49.46 | −10.16 | 23.04 |
| 423 | HOH | O | 4.89 | 19.70 | 5.08 | 21.10 |
| 424 | HOH | O | 14.98 | 28.19 | 13.97 | 24.08 |
| 425 | HOH | O | 9.93 | 49.36 | 26.74 | 23.28 |
| 426 | HOH | O | 20.91 | 22.21 | −14.82 | 23.68 |
| 427 | HOH | O | 9.41 | 61.42 | 20.20 | 22.72 |
| 428 | HOH | O | 3.22 | 25.20 | 9.62 | 21.33 |
| 429 | HOH | O | 12.87 | 22.23 | −15.08 | 23.34 |
| 430 | HOH | O | 25.93 | 58.76 | −9.31 | 22.03 |
| 431 | HOH | O | 16.69 | 63.09 | −4.55 | 21.66 |
| 432 | HOH | O | 17.10 | 20.16 | −17.44 | 23.82 |
| 300 | 458 | C1 | 2.38 | 43.13 | −3.65 | 24.20 |
| 300 | 458 | C2 | 3.30 | 42.69 | −2.65 | 24.07 |
| 300 | 458 | C3 | 1.28 | 42.47 | −4.17 | 24.18 |
| 300 | 458 | O4 | 2.50 | 44.37 | −4.25 | 24.25 |
| 300 | 458 | C5 | 3.34 | 41.56 | −1.89 | 24.16 |
| 300 | 458 | N6 | 4.41 | 43.38 | −2.25 | 24.12 |
| 300 | 458 | C7 | 0.68 | 43.32 | −5.14 | 24.19 |
| 300 | 458 | C8 | 1.48 | 44.49 | −5.15 | 24.18 |
| 300 | 458 | N9 | 4.43 | 41.60 | −1.07 | 24.13 |
| 300 | 458 | N10 | 5.08 | 42.72 | −1.30 | 24.14 |
| 300 | 458 | C11 | −0.45 | 43.24 | −6.02 | 24.24 |
| 300 | 458 | C12 | 1.23 | 45.61 | −5.99 | 24.24 |
| 300 | 458 | C13 | −0.73 | 44.36 | −6.88 | 24.21 |
| 300 | 458 | C14 | 0.10 | 45.52 | −6.86 | 24.22 |
| 253 | ZN2 | ZN | 6.77 | 43.47 | −0.65 | 25.04 |
| 254 | ZN2 | ZN | 5.53 | 40.31 | −0.32 | 19.29 |

TABLE IV
Provides distances between interresidue atoms that are within 5 Ångstroms
apart in an active site of a S. aureus methionine aminopeptidase
for an inhibitor complex with 5-(3-Iodo-phenyl)-1-H-[1,2,3]triazole.

| Atom 1 | Atom 2 | Distance |
|---|---|---|
| 300C1 | 174CD2 | 3.679 |
| 300C1 | 174CD1 | 3.938 |
| 300C1 | 95OG | 4.030 |
| 300C1 | 174CG | 4.403 |
| 300C1 | 56CG | 4.667 |
| 300C1 | 95CB | 4.911 |
| 174CD2 | 300C1 | 38.87 |
| 174CD2 | 300C1 | 93.98 |
| 174CD2 | 300C1 | 19.13 |
| 174CD2 | 300C1 | 112.04 |
| 174CD2 | 300C1 | 90.77 |
| 174CD1 | 300C1 | 62.23 |
| 174CD1 | 300C1 | 20.05 |
| 174CD1 | 300C1 | 101.60 |
| 174CD1 | 300C1 | 54.57 |
| 95OG | 300C1 | 79.66 |
| 95OG | 300C1 | 49.06 |
| 95OG | 300C1 | 14.32 |
| 174CG | 300C1 | 110.30 |
| 174CG | 300C1 | 73.86 |
| 56CG | 300C1 | 63.06 |
| 300C2 | 174CD1 | 3.810 |
| 300C2 | 174CD2 | 4.047 |
| 300C2 | 104OD2 | 4.313 |
| 300C2 | 95OG | 4.340 |
| 300C2 | 104OD1 | 4.348 |
| 300C2 | 93OD1 | 4.424 |
| 300C2 | 174CG | 4.440 |
| 300C2 | 175CE1 | 4.592 |
| 300C2 | 104CG | 4.643 |
| 300C2 | 93OD2 | 4.711 |
| 300C2 | 93CG | 4.779 |
| 300C2 | 95CB | 4.839 |
| 300C2 | 168NE2 | 4.936 |
| 300C2 | 76CE1 | 4.981 |
| 174CD1 | 300C2 | 37.67 |
| 174CD1 | 300C2 | 60.60 |
| 174CD1 | 300C2 | 60.29 |
| 174CD1 | 300C2 | 73.12 |
| 174CD1 | 300C2 | 108.68 |
| 174CD1 | 300C2 | 19.40 |
| 174CD1 | 300C2 | 60.61 |
| 174CD1 | 300C2 | 61.72 |
| 174CD1 | 300C2 | 128.46 |
| 174CD1 | 300C2 | 123.04 |
| 174CD1 | 300C2 | 55.80 |
| 174CD1 | 300C2 | 62.72 |
| 174CD1 | 300C2 | 149.72 |
| 174CD2 | 300C2 | 90.84 |
| 174CD2 | 300C2 | 84.48 |
| 174CD2 | 300C2 | 110.04 |
| 174CD2 | 300C2 | 145.44 |
| 174CD2 | 300C2 | 19.96 |
| 174CD2 | 300C2 | 41.70 |
| 174CD2 | 300C2 | 96.88 |
| 174CD2 | 300C2 | 163.77 |
| 174CD2 | 300C2 | 160.31 |
| 174CD2 | 300C2 | 87.57 |
| 174CD2 | 300C2 | 72.77 |
| 174CD2 | 300C2 | 112.84 |
| 104OD2 | 300C2 | 81.21 |
| 104OD2 | 300C2 | 29.39 |
| 104OD2 | 300C2 | 68.62 |
| 104OD2 | 300C2 | 71.81 |
| 104OD2 | 300C2 | 77.23 |
| 104OD2 | 300C2 | 15.46 |
| 104OD2 | 300C2 | 72.94 |
| 104OD2 | 300C2 | 76.26 |
| 104OD2 | 300C2 | 64.77 |
| 104OD2 | 300C2 | 36.99 |
| 104OD2 | 300C2 | 131.39 |
| 95OG | 300C2 | 62.76 |
| 95OG | 300C2 | 65.66 |
| 95OG | 300C2 | 76.06 |
| 95OG | 300C2 | 120.36 |
| 95OG | 300C2 | 67.82 |
| 95OG | 300C2 | 93.20 |
| 95OG | 300C2 | 78.95 |
| 95OG | 300C2 | 16.63 |
| 95OG | 300C2 | 110.85 |
| 95OG | 300C2 | 139.96 |
| 104OD1 | 300C2 | 41.49 |
| 104OD1 | 300C2 | 90.16 |
| 104OD1 | 300C2 | 106.34 |
| 104OD1 | 300C2 | 15.52 |
| 104OD1 | 300C2 | 55.34 |
| 104OD1 | 300C2 | 52.55 |
| 104OD1 | 300C2 | 46.89 |
| 104OD1 | 300C2 | 66.01 |
| 104OD1 | 300C2 | 132.51 |
| 93OD1 | 300C2 | 127.87 |
| 93OD1 | 300C2 | 144.11 |
| 93OD1 | 300C2 | 56.86 |
| 93OD1 | 300C2 | 27.59 |
| 93OD1 | 300C2 | 14.94 |
| 93OD1 | 300C2 | 58.87 |
| 93OD1 | 300C2 | 100.98 |
| 93OD1 | 300C2 | 101.42 |
| 174CG | 300C2 | 44.45 |
| 174CG | 300C2 | 76.95 |
| 174CG | 300C2 | 144.30 |
| 174CG | 300C2 | 141.84 |
| 174CG | 300C2 | 74.28 |
| 174CG | 300C2 | 60.83 |
| 174CG | 300C2 | 130.32 |
| 175CE1 | 300C2 | 91.08 |
| 175CE1 | 300C2 | 130.38 |
| 175CE1 | 300C2 | 143.91 |
| 175CE1 | 300C2 | 115.63 |
| 175CE1 | 300C2 | 43.13 |
| 175CE1 | 300C2 | 92.75 |
| 104CG | 300C2 | 67.53 |
| 104CG | 300C2 | 67.06 |
| 104CG | 300C2 | 51.19 |
| 104CG | 300C2 | 52.35 |
| 104CG | 300C2 | 138.66 |
| 93OD2 | 300C2 | 15.23 |
| 93OD2 | 300C2 | 85.84 |
| 93OD2 | 300C2 | 93.24 |
| 93OD2 | 300C2 | 79.01 |
| 93CG | 300C2 | 73.55 |
| 93CG | 300C2 | 103.25 |
| 93CG | 300C2 | 86.79 |
| 95CB | 300C2 | 96.40 |
| 95CB | 300C2 | 151.12 |
| 168NE2 | 300C2 | 108.77 |
| 300C3 | 67SG | 4.179 |
| 300C3 | 76CE1 | 4.211 |
| 300C3 | 76NE2 | 4.261 |
| 300C3 | 174CD2 | 4.578 |
| 300C3 | 56CG | 4.626 |
| 300C3 | 76ND1 | 4.673 |
| 300C3 | 76CD2 | 4.774 |
| 67SG | 300C3 | 79.93 |
| 67SG | 300C3 | 87.30 |
| 67SG | 300C3 | 161.25 |
| 67SG | 300C3 | 64.01 |
| 67SG | 300C3 | 63.97 |
| 67SG | 300C3 | 75.35 |
| 76CE1 | 300C3 | 17.93 |
| 76CE1 | 300C3 | 118.10 |
| 76CE1 | 300C3 | 143.92 |
| 76CE1 | 300C3 | 16.05 |
| 76CE1 | 300C3 | 27.36 |
| 76NE2 | 300C3 | 111.44 |
| 76NE2 | 300C3 | 146.59 |
| 76NE2 | 300C3 | 27.26 |
| 76NE2 | 300C3 | 16.26 |
| 174CD2 | 300C3 | 97.93 |
| 174CD2 | 300C3 | 134.15 |
| 174CD2 | 300C3 | 122.89 |
| 56CG | 300C3 | 127.90 |
| 56CG | 300C3 | 130.60 |
| 76ND1 | 300C3 | 26.71 |
| 300C4 | 174CD2 | 3.079 |
| 300C4 | 174CD1 | 3.203 |
| 300C4 | 95OG | 3.309 |
| 300C4 | 174CG | 3.697 |
| 300C4 | 56CG | 4.117 |
| 300C4 | 95CB | 4.307 |
| 300C4 | 56CD | 4.483 |
| 300C4 | 56N | 4.525 |
| 300C4 | 55CB | 4.551 |
| 300C4 | 56CA | 4.603 |
| 300C4 | 174CB | 4.621 |
| 300C4 | 55C | 4.782 |
| 300C4 | 56CB | 4.870 |
| 174CD2 | 300C4 | 47.78 |
| 174CD2 | 300C4 | 124.03 |
| 174CD2 | 300C4 | 23.77 |
| 174CD2 | 300C4 | 149.20 |
| 174CD2 | 300C4 | 112.41 |
| 174CD2 | 300C4 | 134.39 |
| 174CD2 | 300C4 | 118.79 |
| 174CD2 | 300C4 | 95.90 |
| 174CD2 | 300C4 | 119.82 |
| 174CD2 | 300C4 | 29.29 |
| 174CD2 | 300C4 | 103.37 |
| 174CD2 | 300C4 | 137.29 |
| 174CD1 | 300C4 | 78.45 |
| 174CD1 | 300C4 | 24.16 |
| 174CD1 | 300C4 | 131.51 |
| 174CD1 | 300C4 | 65.13 |
| 174CD1 | 300C4 | 112.99 |
| 174CD1 | 300C4 | 112.13 |
| 174CD1 | 300C4 | 71.48 |
| 174CD1 | 300C4 | 127.87 |
| 174CD1 | 300C4 | 30.61 |
| 174CD1 | 300C4 | 98.20 |
| 174CD1 | 300C4 | 139.91 |
| 95OG | 300C4 | 100.94 |
| 95OG | 300C4 | 57.79 |
| 95OG | 300C4 | 15.30 |
| 95OG | 300C4 | 47.45 |
| 95OG | 300C4 | 62.45 |
| 95OG | 300C4 | 45.17 |
| 95OG | 300C4 | 78.07 |
| 95OG | 300C4 | 96.24 |
| 95OG | 300C4 | 65.17 |
| 95OG | 300C4 | 73.22 |
| 174CG | 300C4 | 144.62 |
| 174CG | 300C4 | 88.68 |
| 174CG | 300C4 | 124.94 |
| 174CG | 300C4 | 116.07 |
| 174CG | 300C4 | 81.68 |
| 174CG | 300C4 | 125.45 |
| 174CG | 300C4 | 16.66 |
| 174CG | 300C4 | 99.87 |
| 174CG | 300C4 | 143.13 |
| 56CG | 300C4 | 73.01 |
| 56CG | 300C4 | 19.80 |
| 56CG | 300C4 | 30.94 |
| 56CG | 300C4 | 63.24 |
| 56CG | 300C4 | 30.65 |
| 56CG | 300C4 | 129.25 |
| 56CG | 300C4 | 47.01 |
| 56CG | 300C4 | 16.62 |
| 95CB | 300C4 | 62.34 |
| 95CB | 300C4 | 76.04 |
| 95CB | 300C4 | 50.54 |
| 95CB | 300C4 | 92.61 |
| 95CB | 300C4 | 87.06 |
| 95CB | 300C4 | 76.06 |
| 95CB | 300C4 | 88.52 |
| 56CD | 300C4 | 18.86 |
| 56CD | 300C4 | 43.44 |
| 56CD | 300C4 | 30.79 |
| 56CD | 300C4 | 110.17 |
| 56CD | 300C4 | 31.69 |
| 56CD | 300C4 | 28.60 |
| 56N | 300C4 | 41.27 |
| 56N | 300C4 | 18.42 |
| 56N | 300C4 | 99.66 |
| 56N | 300C4 | 16.30 |
| 56N | 300C4 | 28.51 |
| 55CB | 300C4 | 59.00 |
| 55CB | 300C4 | 68.02 |
| 55CB | 300C4 | 31.10 |
| 55CB | 300C4 | 68.44 |
| 56CA | 300C4 | 108.93 |
| 56CA | 300C4 | 29.74 |
| 56CA | 300C4 | 18.26 |
| 174CB | 300C4 | 83.39 |
| 174CB | 300C4 | 126.48 |
| 55C | 300C4 | 44.03 |
| 300C5 | 175CE1 | 3.532 |
| 300C5 | 174CD1 | 3.593 |
| 300C5 | 104OD2 | 3.613 |
| 300C5 | 168NE2 | 3.619 |
| 300C5 | 174CD2 | 3.821 |
| 300C5 | 174CG | 4.022 |
| 300C5 | 168CE1 | 4.288 |
| 300C5 | 104CG | 4.289 |
| 300C5 | 104OD1 | 4.320 |
| 300C5 | 175NE2 | 4.393 |
| 300C5 | 175ND1 | 4.453 |
| 300C5 | 168CD2 | 4.498 |
| 300C5 | 202OE1 | 4.829 |
| 300C5 | 233OE2 | 4.868 |
| 175CE1 | 300C5 | 74.10 |
| 175CE1 | 300C5 | 102.23 |
| 175CE1 | 300C5 | 58.91 |
| 175CE1 | 300C5 | 49.97 |
| 175CE1 | 300C5 | 53.35 |
| 175CE1 | 300C5 | 57.33 |
| 175CE1 | 300C5 | 114.55 |
| 175CE1 | 300C5 | 131.18 |
| 175CE1 | 300C5 | 14.62 |
| 175CE1 | 300C5 | 13.75 |
| 175CE1 | 300C5 | 50.49 |
| 175CE1 | 300C5 | 86.19 |
| 175CE1 | 300C5 | 114.50 |
| 174CD1 | 300C5 | 69.77 |
| 174CD1 | 300C5 | 80.34 |
| 174CD1 | 300C5 | 40.03 |
| 174CD1 | 300C5 | 22.15 |
| 174CD1 | 300C5 | 63.75 |
| 174CD1 | 300C5 | 67.11 |
| 174CD1 | 300C5 | 75.50 |
| 174CD1 | 300C5 | 87.86 |
| 174CD1 | 300C5 | 60.36 |
| 174CD1 | 300C5 | 89.95 |
| 174CD1 | 300C5 | 122.27 |
| 174CD1 | 300C5 | 103.38 |
| 104OD2 | 300C5 | 48.89 |
| 104OD2 | 300C5 | 106.49 |
| 104OD2 | 300C5 | 84.33 |
| 104OD2 | 300C5 | 45.07 |
| 104OD2 | 300C5 | 15.33 |
| 104OD2 | 300C5 | 30.53 |
| 104OD2 | 300C5 | 103.16 |
| 104OD2 | 300C5 | 96.64 |
| 104OD2 | 300C5 | 63.15 |
| 104OD2 | 300C5 | 62.28 |
| 104OD2 | 300C5 | 33.68 |
| 168NE2 | 300C5 | 92.51 |
| 168NE2 | 300C5 | 76.98 |
| 168NE2 | 300C5 | 16.60 |
| 168NE2 | 300C5 | 64.09 |
| 168NE2 | 300C5 | 79.05 |
| 168NE2 | 300C5 | 55.42 |
| 168NE2 | 300C5 | 60.08 |
| 168NE2 | 300C5 | 15.06 |
| 168NE2 | 300C5 | 44.18 |
| 168NE2 | 300C5 | 56.34 |
| 174CD2 | 300C5 | 22.17 |
| 174CD2 | 300C5 | 79.92 |
| 174CD2 | 300C5 | 106.76 |
| 174CD2 | 300C5 | 115.25 |
| 174CD2 | 300C5 | 64.07 |
| 174CD2 | 300C5 | 39.17 |
| 174CD2 | 300C5 | 92.61 |
| 174CD2 | 300C5 | 133.13 |
| 174CD2 | 300C5 | 139.07 |
| 174CG | 300C5 | 62.02 |
| 174CG | 300C5 | 85.66 |
| 174CG | 300C5 | 96.42 |
| 174CG | 300C5 | 67.72 |
| 174CG | 300C5 | 39.70 |
| 174CG | 300C5 | 81.66 |
| 174CG | 300C5 | 121.02 |
| 174CG | 300C5 | 117.05 |
| 168CE1 | 300C5 | 58.70 |
| 168CE1 | 300C5 | 75.16 |
| 168CE1 | 300C5 | 58.81 |
| 168CE1 | 300C5 | 53.96 |
| 168CE1 | 300C5 | 28.78 |
| 168CE1 | 300C5 | 59.99 |
| 168CE1 | 300C5 | 63.52 |
| 104CG | 300C5 | 16.67 |
| 104CG | 300C5 | 117.46 |
| 104CG | 300C5 | 106.71 |
| 104CG | 300C5 | 78.47 |
| 104CG | 300C5 | 73.75 |
| 104CG | 300C5 | 38.78 |
| 104OD1 | 300C5 | 133.68 |
| 104OD1 | 300C5 | 122.67 |
| 104OD1 | 300C5 | 92.63 |
| 104OD1 | 300C5 | 78.88 |
| 104OD1 | 300C5 | 40.21 |
| 175NE2 | 300C5 | 28.03 |
| 175NE2 | 300C5 | 43.66 |
| 175NE2 | 300C5 | 74.27 |
| 175NE2 | 300C5 | 107.09 |
| 175ND1 | 300C5 | 55.46 |
| 175ND1 | 300C5 | 94.63 |
| 175ND1 | 300C5 | 116.21 |
| 168CD2 | 300C5 | 40.71 |
| 168CD2 | 300C5 | 64.23 |
| 202OE1 | 300C5 | 38.87 |
| 300N6 | 93OD1 | 3.143 |
| 300N6 | 93OD2 | 3.381 |
| 300N6 | 93CG | 3.432 |
| 300N6 | 104OD1 | 3.591 |
| 300N6 | 104OD2 | 4.058 |
| 300N6 | 104CG | 4.184 |
| 300N6 | 95OG | 4.376 |
| 300N6 | 233OE1 | 4.497 |
| 300N6 | 93CB | 4.567 |
| 300N6 | 174CD1 | 4.658 |
| 300N6 | 95CB | 4.680 |
| 300N6 | 233OE2 | 4.750 |
| 93OD1 | 300N6 | 39.13 |
| 93OD1 | 300N6 | 21.29 |
| 93OD1 | 300N6 | 54.47 |
| 93OD1 | 300N6 | 85.29 |
| 93OD1 | 300N6 | 70.65 |
| 93OD1 | 300N6 | 76.44 |
| 93OD1 | 300N6 | 44.59 |
| 93OD1 | 300N6 | 29.20 |
| 93OD1 | 300N6 | 117.00 |
| 93OD1 | 300N6 | 68.18 |
| 93OD1 | 300N6 | 70.83 |
| 93OD2 | 300N6 | 21.29 |
| 93OD2 | 300N6 | 74.41 |
| 93OD2 | 300N6 | 92.03 |
| 93OD2 | 300N6 | 86.14 |
| 93OD2 | 300N6 | 115.48 |
| 93OD2 | 300N6 | 37.50 |
| 93OD2 | 300N6 | 30.89 |
| 93OD2 | 300N6 | 145.34 |
| 93OD2 | 300N6 | 106.46 |
| 93OD2 | 300N6 | 60.37 |
| 93CG | 300N6 | 70.57 |
| 93CG | 300N6 | 97.02 |
| 93CG | 300N6 | 85.64 |
| 93CG | 300N6 | 95.35 |
| 93CG | 300N6 | 45.40 |
| 93CG | 300N6 | 14.73 |
| 93CG | 300N6 | 137.95 |
| 93CG | 300N6 | 89.07 |
| 93CG | 300N6 | 72.37 |
| 104OD1 | 300N6 | 32.66 |
| 104OD1 | 300N6 | 16.29 |
| 104OD1 | 300N6 | 68.37 |
| 104OD1 | 300N6 | 41.24 |
| 104OD1 | 300N6 | 82.86 |
| 104OD1 | 300N6 | 71.17 |
| 104OD1 | 300N6 | 50.82 |
| 104OD1 | 300N6 | 42.33 |
| 104OD2 | 300N6 | 17.34 |
| 104OD2 | 300N6 | 83.69 |
| 104OD2 | 300N6 | 54.67 |
| 104OD2 | 300N6 | 111.21 |
| 104OD2 | 300N6 | 55.92 |
| 104OD2 | 300N6 | 68.14 |
| 104OD2 | 300N6 | 35.07 |
| 104CG | 300N6 | 71.74 |
| 104CG | 300N6 | 49.60 |
| 104CG | 300N6 | 98.62 |
| 104CG | 300N6 | 59.32 |
| 104CG | 300N6 | 54.76 |
| 104CG | 300N6 | 39.82 |
| 95OG | 300N6 | 103.90 |
| 95OG | 300N6 | 92.79 |
| 95OG | 300N6 | 54.13 |
| 95OG | 300N6 | 17.58 |
| 95OG | 300N6 | 110.22 |
| 233OE1 | 300N6 | 60.09 |
| 233OE1 | 300N6 | 108.68 |
| 233OE1 | 300N6 | 87.80 |
| 233OE1 | 300N6 | 27.34 |
| 93CB | 300N6 | 143.39 |
| 93CB | 300N6 | 90.88 |
| 93CB | 300N6 | 86.89 |
| 174CD1 | 300N6 | 52.74 |
| 174CD1 | 300N6 | 90.57 |
| 95CB | 300N6 | 92.67 |
| 300C7 | 56CG | 4.051 |
| 300C7 | 56CB | 4.136 |
| 300C7 | 67SG | 4.162 |
| 300C7 | 60CG | 4.251 |
| 300C7 | 56CA | 4.290 |
| 300C7 | 76NE2 | 4.772 |
| 300C7 | 174CD2 | 4.915 |
| 300C7 | 76CD2 | 4.964 |
| 56CG | 300C7 | 21.05 |
| 56CG | 300C7 | 69.49 |
| 56CG | 300C7 | 100.39 |
| 56CG | 300C7 | 32.59 |
| 56CG | 300C7 | 149.40 |
| 56CG | 300C7 | 101.04 |
| 56CG | 300C7 | 142.44 |
| 56CB | 300C7 | 73.79 |
| 56CB | 300C7 | 85.83 |
| 56CB | 300C7 | 20.77 |
| 56CB | 300C7 | 151.69 |
| 56CB | 300C7 | 110.08 |
| 56CB | 300C7 | 137.33 |
| 67SG | 300C7 | 144.99 |
| 67SG | 300C7 | 94.55 |
| 67SG | 300C7 | 81.09 |
| 67SG | 300C7 | 144.17 |
| 67SG | 300C7 | 73.41 |
| 60CG | 300C7 | 68.06 |
| 60CG | 300C7 | 108.70 |
| 60CG | 300C7 | 69.46 |
| 60CG | 300C7 | 106.64 |
| 56CA | 300C7 | 166.68 |
| 56CA | 300C7 | 92.98 |
| 56CA | 300C7 | 150.76 |
| 76NE2 | 300C7 | 97.90 |
| 76NE2 | 300C7 | 16.08 |
| 174CD2 | 300C7 | 112.52 |
| 300C8 | 56CA | 3.364 |
| 300C8 | 56CG | 3.482 |
| 300C8 | 56N | 3.515 |
| 300C8 | 174CD2 | 3.578 |
| 300C8 | 55C | 3.794 |
| 300C8 | 95OG | 3.823 |
| 300C8 | 56CD | 3.833 |
| 300C8 | 56CB | 3.872 |
| 300C8 | 55O | 3.889 |
| 300C8 | 60CG | 4.116 |
| 300C8 | 174CD1 | 4.164 |
| 300C8 | 55CB | 4.276 |
| 300C8 | 174CG | 4.410 |
| 300C8 | 56C | 4.628 |
| 300C8 | 55CA | 4.695 |
| 300C8 | 56O | 4.906 |
| 300C8 | 60OE2 | 4.967 |
| 300C8 | 174CB | 4.987 |
| 56CA | 300C8 | 40.13 |
| 56CA | 300C8 | 24.43 |
| 56CA | 300C8 | 148.99 |
| 56CA | 300C8 | 38.85 |
| 56CA | 300C8 | 89.78 |
| 56CA | 300C8 | 38.52 |
| 56CA | 300C8 | 23.00 |
| 56CA | 300C8 | 42.98 |
| 56CA | 300C8 | 78.72 |
| 56CA | 300C8 | 138.18 |
| 56CA | 300C8 | 71.18 |
| 56CA | 300C8 | 143.40 |
| 56CA | 300C8 | 12.37 |
| 56CA | 300C8 | 53.45 |
| 56CA | 300C8 | 26.21 |
| 56CA | 300C8 | 96.90 |
| 56CA | 300C8 | 126.90 |
| 56CG | 300C8 | 39.05 |
| 56CG | 300C8 | 159.09 |
| 56CG | 300C8 | 59.14 |
| 56CG | 300C8 | 59.87 |
| 56CG | 300C8 | 23.38 |
| 56CG | 300C8 | 22.71 |
| 56CG | 300C8 | 73.69 |
| 56CG | 300C8 | 113.93 |
| 56CG | 300C8 | 121.65 |
| 56CG | 300C8 | 71.16 |
| 56CG | 300C8 | 140.99 |
| 56CG | 300C8 | 52.31 |
| 56CG | 300C8 | 62.94 |
| 56CG | 300C8 | 64.61 |
| 56CG | 300C8 | 136.67 |
| 56CG | 300C8 | 136.95 |
| 56N | 300C8 | 136.40 |
| 56N | 300C8 | 20.74 |
| 56N | 300C8 | 69.52 |
| 56N | 300C8 | 22.66 |
| 56N | 300C8 | 36.48 |
| 56N | 300C8 | 34.91 |
| 56N | 300C8 | 96.57 |
| 56N | 300C8 | 114.10 |
| 56N | 300C8 | 47.25 |
| 56N | 300C8 | 123.35 |
| 56N | 300C8 | 32.19 |
| 56N | 300C8 | 30.81 |
| 56N | 300C8 | 46.18 |
| 56N | 300C8 | 106.13 |
| 56N | 300C8 | 109.35 |
| 174CD2 | 300C8 | 116.13 |
| 174CD2 | 300C8 | 99.28 |
| 174CD2 | 300C8 | 141.25 |
| 174CD2 | 300C8 | 171.87 |
| 174CD2 | 300C8 | 107.01 |
| 174CD2 | 300C8 | 85.76 |
| 174CD2 | 300C8 | 37.45 |
| 174CD2 | 300C8 | 93.73 |
| 174CD2 | 300C8 | 18.45 |
| 174CD2 | 300C8 | 139.30 |
| 174CD2 | 300C8 | 106.59 |
| 174CD2 | 300C8 | 130.49 |
| 174CD2 | 300C8 | 60.17 |
| 174CD2 | 300C8 | 27.49 |
| 55C | 300C8 | 72.99 |
| 55C | 300C8 | 39.00 |
| 55C | 300C8 | 56.33 |
| 55C | 300C8 | 18.39 |
| 55C | 300C8 | 91.45 |
| 55C | 300C8 | 100.49 |
| 55C | 300C8 | 35.64 |
| 55C | 300C8 | 105.15 |
| 55C | 300C8 | 40.40 |
| 55C | 300C8 | 16.83 |
| 55C | 300C8 | 50.68 |
| 55C | 300C8 | 92.17 |
| 55C | 300C8 | 89.79 |
| 95OG | 300C8 | 51.29 |
| 95OG | 300C8 | 81.74 |
| 95OG | 300C8 | 89.88 |
| 95OG | 300C8 | 164.34 |
| 95OG | 300C8 | 61.90 |
| 95OG | 300C8 | 46.59 |
| 95OG | 300C8 | 81.81 |
| 95OG | 300C8 | 100.70 |
| 95OG | 300C8 | 59.32 |
| 95OG | 300C8 | 115.17 |
| 95OG | 300C8 | 146.80 |
| 95OG | 300C8 | 84.18 |
| 56CD | 300C8 | 35.36 |
| 56CD | 300C8 | 56.17 |
| 56CD | 300C8 | 116.79 |
| 56CD | 300C8 | 107.38 |
| 56CD | 300C8 | 48.33 |
| 56CD | 300C8 | 123.40 |
| 56CD | 300C8 | 49.90 |
| 56CD | 300C8 | 39.81 |
| 56CD | 300C8 | 64.35 |
| 56CD | 300C8 | 128.78 |
| 56CD | 300C8 | 115.02 |
| 56CB | 300C8 | 64.88 |
| 56CB | 300C8 | 91.22 |
| 56CB | 300C8 | 142.25 |
| 56CB | 300C8 | 81.07 |
| 56CB | 300C8 | 158.36 |
| 56CB | 300C8 | 33.27 |
| 56CB | 300C8 | 66.90 |
| 56CB | 300C8 | 43.35 |
| 56CB | 300C8 | 115.01 |
| 56CB | 300C8 | 145.83 |
| 55O | 300C8 | 74.46 |
| 55O | 300C8 | 103.04 |
| 55O | 300C8 | 47.15 |
| 55O | 300C8 | 101.14 |
| 55O | 300C8 | 38.47 |
| 55O | 300C8 | 30.73 |
| 55O | 300C8 | 43.20 |
| 55O | 300C8 | 73.89 |
| 55O | 300C8 | 84.19 |
| 60CG | 300C8 | 121.13 |
| 60CG | 300C8 | 118.63 |
| 60CG | 300C8 | 101.07 |
| 60CG | 300C8 | 66.93 |
| 60CG | 300C8 | 105.05 |
| 60CG | 300C8 | 52.56 |
| 60CG | 300C8 | 28.38 |
| 60CG | 300C8 | 94.18 |
| 174CD1 | 300C8 | 67.01 |
| 174CD1 | 300C8 | 20.20 |
| 174CD1 | 300C8 | 140.42 |
| 174CD1 | 300C8 | 84.90 |
| 174CD1 | 300C8 | 145.29 |
| 174CD1 | 300C8 | 93.20 |
| 174CD1 | 300C8 | 29.70 |
| 55CB | 300C8 | 77.41 |
| 55CB | 300C8 | 75.50 |
| 55CB | 300C8 | 18.83 |
| 55CB | 300C8 | 86.30 |
| 55CB | 300C8 | 105.28 |
| 55CB | 300C8 | 66.76 |
| 174CG | 300C8 | 138.93 |
| 174CG | 300C8 | 92.54 |
| 174CG | 300C8 | 136.17 |
| 174CG | 300C8 | 73.50 |
| 174CG | 300C8 | 17.14 |
| 56C | 300C8 | 56.79 |
| 56C | 300C8 | 14.49 |
| 56C | 300C8 | 84.57 |
| 56C | 300C8 | 121.79 |
| 55CA | 300C8 | 67.51 |
| 55CA | 300C8 | 99.40 |
| 55CA | 300C8 | 79.13 |
| 56O | 300C8 | 72.09 |
| 56O | 300C8 | 119.94 |
| 60OE2 | 300C8 | 65.82 |
| 300N9 | 104OD2 | 2.874 |
| 300N9 | 168NE2 | 3.218 |
| 300N9 | 233OE2 | 3.558 |
| 300N9 | 104OD1 | 3.610 |
| 300N9 | 104CG | 3.612 |
| 300N9 | 202OE1 | 3.749 |
| 300N9 | 168CE1 | 4.092 |
| 300N9 | 202OE2 | 4.127 |
| 300N9 | 168CD2 | 4.147 |
| 300N9 | 93OD2 | 4.216 |
| 300N9 | 202CD | 4.312 |
| 300N9 | 175CE1 | 4.316 |
| 300N9 | 233OE1 | 4.326 |
| 300N9 | 233CD | 4.362 |
| 300N9 | 174CD1 | 4.390 |
| 300N9 | 93OD1 | 4.504 |
| 300N9 | 93CG | 4.740 |
| 300N9 | 175NE2 | 4.947 |
| 104OD2 | 300N9 | 58.52 |
| 104OD2 | 300N9 | 48.91 |
| 104OD2 | 300N9 | 37.49 |
| 104OD2 | 300N9 | 17.98 |
| 104OD2 | 300N9 | 84.22 |
| 104OD2 | 300N9 | 48.93 |
| 104OD2 | 300N9 | 93.99 |
| 104OD2 | 300N9 | 73.29 |
| 104OD2 | 300N9 | 96.73 |
| 104OD2 | 300N9 | 85.15 |
| 104OD2 | 300N9 | 99.37 |
| 104OD2 | 300N9 | 62.73 |
| 104OD2 | 300N9 | 53.68 |
| 104OD2 | 300N9 | 65.29 |
| 104OD2 | 300N9 | 80.47 |
| 104OD2 | 300N9 | 91.94 |
| 104OD2 | 300N9 | 103.92 |
| 168NE2 | 300N9 | 75.46 |
| 168NE2 | 300N9 | 95.99 |
| 168NE2 | 300N9 | 76.38 |
| 168NE2 | 300N9 | 57.24 |
| 168NE2 | 300N9 | 15.65 |
| 168NE2 | 300N9 | 87.80 |
| 168NE2 | 300N9 | 15.93 |
| 168NE2 | 300N9 | 140.91 |
| 168NE2 | 300N9 | 71.02 |
| 168NE2 | 300N9 | 53.26 |
| 168NE2 | 300N9 | 105.18 |
| 168NE2 | 300N9 | 88.90 |
| 168NE2 | 300N9 | 73.59 |
| 168NE2 | 300N9 | 138.21 |
| 168NE2 | 300N9 | 145.71 |
| 168NE2 | 300N9 | 49.96 |
| 233OE2 | 300N9 | 53.02 |
| 233OE2 | 300N9 | 51.03 |
| 233OE2 | 300N9 | 52.33 |
| 233OE2 | 300N9 | 78.24 |
| 233OE2 | 300N9 | 46.67 |
| 233OE2 | 300N9 | 80.31 |
| 233OE2 | 300N9 | 65.76 |
| 233OE2 | 300N9 | 43.83 |
| 233OE2 | 300N9 | 128.55 |
| 233OE2 | 300N9 | 30.45 |
| 233OE2 | 300N9 | 13.88 |
| 233OE2 | 300N9 | 114.12 |
| 233OE2 | 300N9 | 71.25 |
| 233OE2 | 300N9 | 71.58 |
| 233OE2 | 300N9 | 121.57 |
| 104OD1 | 300N9 | 19.91 |
| 104OD1 | 300N9 | 104.64 |
| 104OD1 | 300N9 | 85.68 |
| 104OD1 | 300N9 | 95.12 |
| 104OD1 | 300N9 | 110.39 |
| 104OD1 | 300N9 | 64.70 |
| 104OD1 | 300N9 | 96.62 |
| 104OD1 | 300N9 | 128.87 |
| 104OD1 | 300N9 | 42.80 |
| 104OD1 | 300N9 | 46.29 |
| 104OD1 | 300N9 | 74.43 |
| 104OD1 | 300N9 | 43.31 |
| 104OD1 | 300N9 | 56.23 |
| 104OD1 | 300N9 | 138.27 |
| 104CG | 300N9 | 96.40 |
| 104CG | 300N9 | 65.79 |
| 104CG | 300N9 | 97.58 |
| 104CG | 300N9 | 91.26 |
| 104CG | 300N9 | 82.85 |
| 104CG | 300N9 | 93.29 |
| 104CG | 300N9 | 112.18 |
| 104CG | 300N9 | 53.88 |
| 104CG | 300N9 | 50.29 |
| 104CG | 300N9 | 65.79 |
| 104CG | 300N9 | 63.22 |
| 104CG | 300N9 | 75.78 |
| 104CG | 300N9 | 119.39 |
| 202OE1 | 300N9 | 71.36 |
| 202OE1 | 300N9 | 31.63 |
| 202OE1 | 300N9 | 48.43 |
| 202OE1 | 300N9 | 93.39 |
| 202OE1 | 300N9 | 15.80 |
| 202OE1 | 300N9 | 91.49 |
| 202OE1 | 300N9 | 75.39 |
| 202OE1 | 300N9 | 62.68 |
| 202OE1 | 300N9 | 130.62 |
| 202OE1 | 300N9 | 115.16 |
| 202OE1 | 300N9 | 106.81 |
| 202OE1 | 300N9 | 78.47 |
| 168CE1 | 300N9 | 100.44 |
| 168CE1 | 300N9 | 30.86 |
| 168CE1 | 300N9 | 142.46 |
| 168CE1 | 300N9 | 83.87 |
| 168CE1 | 300N9 | 53.79 |
| 168CE1 | 300N9 | 105.05 |
| 168CE1 | 300N9 | 90.25 |
| 168CE1 | 300N9 | 59.28 |
| 168CE1 | 300N9 | 128.99 |
| 168CE1 | 300N9 | 140.78 |
| 168CE1 | 300N9 | 55.30 |
| 202OE2 | 300N9 | 80.06 |
| 202OE2 | 300N9 | 62.37 |
| 202OE2 | 300N9 | 16.78 |
| 202OE2 | 300N9 | 119.41 |
| 202OE2 | 300N9 | 54.57 |
| 202OE2 | 300N9 | 49.09 |
| 202OE2 | 300N9 | 157.23 |
| 202OE2 | 300N9 | 87.36 |
| 202OE2 | 300N9 | 76.49 |
| 202OE2 | 300N9 | 105.16 |
| 168CD2 | 300N9 | 140.60 |
| 168CD2 | 300N9 | 63.81 |
| 168CD2 | 300N9 | 49.31 |
| 168CD2 | 300N9 | 110.72 |
| 168CD2 | 300N9 | 94.18 |
| 168CD2 | 300N9 | 84.70 |
| 168CD2 | 300N9 | 149.86 |
| 168CD2 | 300N9 | 151.28 |
| 168CD2 | 300N9 | 41.49 |
| 93OD2 | 300N9 | 77.62 |
| 93OD2 | 300N9 | 163.57 |
| 93OD2 | 300N9 | 37.44 |
| 93OD2 | 300N9 | 52.80 |
| 93OD2 | 300N9 | 126.45 |
| 93OD2 | 300N9 | 28.93 |
| 93OD2 | 300N9 | 14.72 |
| 93OD2 | 300N9 | 156.79 |
| 202CD | 300N9 | 106.94 |
| 202CD | 300N9 | 61.82 |
| 202CD | 300N9 | 51.34 |
| 202CD | 300N9 | 142.29 |
| 202CD | 300N9 | 99.92 |
| 202CD | 300N9 | 91.03 |
| 202CD | 300N9 | 93.50 |
| 175CE1 | 300N9 | 158.35 |
| 175CE1 | 300N9 | 142.15 |
| 175CE1 | 300N9 | 59.08 |
| 175CE1 | 300N9 | 153.05 |
| 175CE1 | 300N9 | 159.41 |
| 175CE1 | 300N9 | 14.43 |
| 233OE1 | 300N9 | 16.58 |
| 233OE1 | 300N9 | 117.17 |
| 233OE1 | 300N9 | 41.84 |
| 233OE1 | 300N9 | 41.20 |
| 233OE1 | 300N9 | 151.60 |
| 233CD | 300N9 | 116.05 |
| 233CD | 300N9 | 57.65 |
| 233CD | 300N9 | 57.76 |
| 233CD | 300N9 | 135.37 |
| 174CD1 | 300N9 | 97.74 |
| 174CD1 | 300N9 | 111.84 |
| 174CD1 | 300N9 | 72.98 |
| 93OD1 | 300N9 | 15.23 |
| 93OD1 | 300N9 | 166.22 |
| 93CG | 300N9 | 163.81 |
| 300N10 | 93OD2 | 2.996 |
| 300N10 | 104OD1 | 3.091 |
| 300N10 | 104OD2 | 3.230 |
| 300N10 | 93OD1 | 3.241 |
| 300N10 | 93CG | 3.428 |
| 300N10 | 233OE2 | 3.474 |
| 300N10 | 233OE1 | 3.524 |
| 300N10 | 104CG | 3.546 |
| 300N10 | 233CD | 3.916 |
| 300N10 | 202OE2 | 4.126 |
| 300N10 | 168NE2 | 4.399 |
| 300N10 | 202OE1 | 4.402 |
| 300N10 | 202CD | 4.628 |
| 300N10 | 93CB | 4.804 |
| 300N10 | 174CD1 | 4.968 |
| 93OD2 | 300N10 | 87.73 |
| 93OD2 | 300N10 | 119.26 |
| 93OD2 | 300N10 | 41.00 |
| 93OD2 | 300N10 | 21.28 |
| 93OD2 | 300N10 | 81.90 |
| 93OD2 | 300N10 | 48.95 |
| 93OD2 | 300N10 | 104.95 |
| 93OD2 | 300N10 | 65.46 |
| 93OD2 | 300N10 | 72.75 |
| 93OD2 | 300N10 | 142.31 |
| 93OD2 | 300N10 | 101.68 |
| 93OD2 | 300N10 | 86.24 |
| 93OD2 | 300N10 | 24.15 |
| 93OD2 | 300N10 | 148.51 |
| 104OD1 | 300N10 | 40.61 |
| 104OD1 | 300N10 | 58.72 |
| 104OD1 | 300N10 | 76.82 |
| 104OD1 | 300N10 | 57.98 |
| 104OD1 | 300N10 | 52.89 |
| 104OD1 | 300N10 | 20.23 |
| 104OD1 | 300N10 | 52.94 |
| 104OD1 | 300N10 | 103.95 |
| 104OD1 | 300N10 | 83.47 |
| 104OD1 | 300N10 | 100.58 |
| 104OD1 | 300N10 | 98.53 |
| 104OD1 | 300N10 | 84.28 |
| 104OD1 | 300N10 | 70.14 |
| 104OD2 | 300N10 | 99.13 |
| 104OD2 | 300N10 | 115.31 |
| 104OD2 | 300N10 | 47.97 |
| 104OD2 | 300N10 | 71.38 |
| 104OD2 | 300N10 | 20.56 |
| 104OD2 | 300N10 | 58.20 |
| 104OD2 | 300N10 | 88.90 |
| 104OD2 | 300N10 | 42.87 |
| 104OD2 | 300N10 | 70.13 |
| 104OD2 | 300N10 | 76.28 |
| 104OD2 | 300N10 | 123.72 |
| 104OD2 | 300N10 | 55.61 |
| 93OD1 | 300N10 | 21.32 |
| 93OD1 | 300N10 | 90.16 |
| 93OD1 | 300N10 | 55.45 |
| 93OD1 | 300N10 | 78.95 |
| 93OD1 | 300N10 | 72.70 |
| 93OD1 | 300N10 | 107.58 |
| 93OD1 | 300N10 | 141.76 |
| 93OD1 | 300N10 | 131.25 |
| 93OD1 | 300N10 | 117.06 |
| 93OD1 | 300N10 | 26.33 |
| 93OD1 | 300N10 | 107.53 |
| 93CG | 300N10 | 91.55 |
| 93CG | 300N10 | 55.04 |
| 93CG | 300N10 | 96.56 |
| 93CG | 300N10 | 73.46 |
| 93CG | 300N10 | 93.21 |
| 93CG | 300N10 | 153.23 |
| 93CG | 300N10 | 121.29 |
| 93CG | 300N10 | 105.78 |
| 93CG | 300N10 | 9.22 |
| 93CG | 300N10 | 127.63 |
| 233OE2 | 300N10 | 36.62 |
| 233OE2 | 300N10 | 52.20 |
| 233OE2 | 300N10 | 18.18 |
| 233OE2 | 300N10 | 47.00 |
| 233OE2 | 300N10 | 62.41 |
| 233OE2 | 300N10 | 46.54 |
| 233OE2 | 300N10 | 40.73 |
| 233OE2 | 300N10 | 100.21 |
| 233OE2 | 300N10 | 103.32 |
| 233OE1 | 300N10 | 62.21 |
| 233OE1 | 300N10 | 18.45 |
| 233OE1 | 300N10 | 60.34 |
| 233OE1 | 300N10 | 98.51 |
| 233OE1 | 300N10 | 76.57 |
| 233OE1 | 300N10 | 64.29 |
| 233OE1 | 300N10 | 63.89 |
| 233OE1 | 300N10 | 121.40 |
| 104CG | 300N10 | 54.91 |
| 104CG | 300N10 | 98.68 |
| 104CG | 300N10 | 63.34 |
| 104CG | 300N10 | 86.64 |
| 104CG | 300N10 | 89.02 |
| 104CG | 300N10 | 104.35 |
| 104CG | 300N10 | 59.40 |
| 233CD | 300N10 | 52.05 |
| 233CD | 300N10 | 80.24 |
| 233CD | 300N10 | 61.15 |
| 233CD | 300N10 | 51.37 |
| 233CD | 300N10 | 82.23 |
| 233CD | 300N10 | 112.87 |
| 202OE2 | 300N10 | 74.00 |
| 202OE2 | 300N10 | 29.35 |
| 202OE2 | 300N10 | 14.98 |
| 202OE2 | 300N10 | 96.76 |
| 202OE2 | 300N10 | 133.12 |
| 168NE2 | 300N10 | 45.02 |
| 168NE2 | 300N10 | 59.20 |
| 168NE2 | 300N10 | 162.34 |
| 168NE2 | 300N10 | 59.19 |
| 202OE1 | 300N10 | 15.53 |
| 202OE1 | 300N10 | 125.82 |
| 202OE1 | 300N10 | 104.17 |
| 202CD | 300N10 | 110.39 |
| 202CD | 300N10 | 118.20 |
| 93CB | 300N10 | 127.36 |
| 300C11 | 56CA | 3.156 |
| 300C11 | 56CB | 3.421 |
| 300C11 | 56CG | 3.445 |
| 300C11 | 60CG | 3.523 |
| 300C11 | 56N | 3.858 |
| 300C11 | 56CD | 4.223 |
| 300C11 | 56C | 4.268 |
| 300C11 | 60CB | 4.320 |
| 300C11 | 56O | 4.346 |
| 300C11 | 55O | 4.402 |
| 300C11 | 55C | 4.414 |
| 300C11 | 174CD2 | 4.492 |
| 300C11 | 60CD | 4.721 |
| 300C11 | 95OG | 4.846 |
| 300C11 | 62CB | 4.869 |
| 300C11 | 60OE2 | 4.986 |
| 56CA | 300C11 | 26.44 |
| 56CA | 300C11 | 41.44 |
| 56CA | 300C11 | 91.21 |
| 56CA | 300C11 | 21.20 |
| 56CA | 300C11 | 34.51 |
| 56CA | 300C11 | 16.30 |
| 56CA | 300C11 | 78.90 |
| 56CA | 300C11 | 32.73 |
| 56CA | 300C11 | 37.53 |
| 56CA | 300C11 | 32.06 |
| 56CA | 300C11 | 121.02 |
| 56CA | 300C11 | 99.64 |
| 56CA | 300C11 | 75.60 |
| 56CA | 300C11 | 54.27 |
| 56CA | 300C11 | 99.48 |
| 56CB | 300C11 | 25.19 |
| 56CB | 300C11 | 110.66 |
| 56CB | 300C11 | 36.84 |
| 56CB | 300C11 | 33.62 |
| 56CB | 300C11 | 36.50 |
| 56CB | 300C11 | 94.13 |
| 56CB | 300C11 | 49.89 |
| 56CB | 300C11 | 62.84 |
| 56CB | 300C11 | 53.20 |
| 56CB | 300C11 | 139.41 |
| 56CB | 300C11 | 121.61 |
| 56CB | 300C11 | 72.76 |
| 56CB | 300C11 | 47.79 |
| 56CB | 300C11 | 124.59 |
| 56CG | 300C11 | 132.54 |
| 56CG | 300C11 | 36.80 |
| 56CG | 300C11 | 19.74 |
| 56CG | 300C11 | 56.45 |
| 56CG | 300C11 | 118.15 |
| 56CG | 300C11 | 72.08 |
| 56CG | 300C11 | 67.51 |
| 56CG | 300C11 | 52.80 |
| 56CG | 300C11 | 121.45 |
| 56CG | 300C11 | 140.76 |
| 56CG | 300C11 | 48.84 |
| 56CG | 300C11 | 71.64 |
| 56CG | 300C11 | 137.24 |
| 60CG | 300C11 | 101.27 |
| 60CG | 300C11 | 121.63 |
| 60CG | 300C11 | 76.14 |
| 60CG | 300C11 | 19.01 |
| 60CG | 300C11 | 61.01 |
| 60CG | 300C11 | 74.48 |
| 60CG | 300C11 | 90.43 |
| 60CG | 300C11 | 80.87 |
| 60CG | 300C11 | 12.94 |
| 60CG | 300C11 | 139.52 |
| 60CG | 300C11 | 76.93 |
| 60CG | 300C11 | 25.79 |
| 56N | 300C11 | 20.41 |
| 56N | 300C11 | 35.34 |
| 56N | 300C11 | 93.75 |
| 56N | 300C11 | 50.73 |
| 56N | 300C11 | 30.72 |
| 56N | 300C11 | 17.05 |
| 56N | 300C11 | 103.86 |
| 56N | 300C11 | 105.87 |
| 56N | 300C11 | 56.20 |
| 56N | 300C11 | 75.09 |
| 56N | 300C11 | 100.46 |
| 56CD | 300C11 | 50.78 |
| 56CD | 300C11 | 112.67 |
| 56CD | 300C11 | 67.17 |
| 56CD | 300C11 | 49.81 |
| 56CD | 300C11 | 34.20 |
| 56CD | 300C11 | 106.64 |
| 56CD | 300C11 | 126.01 |
| 56CD | 300C11 | 42.17 |
| 56CD | 300C11 | 80.55 |
| 56CD | 300C11 | 119.15 |
| 56C | 300C11 | 62.76 |
| 56C | 300C11 | 16.43 |
| 56C | 300C11 | 38.92 |
| 56C | 300C11 | 40.55 |
| 56C | 300C11 | 123.07 |
| 56C | 300C11 | 85.77 |
| 56C | 300C11 | 91.25 |
| 56C | 300C11 | 45.19 |
| 56C | 300C11 | 88.19 |
| 60CB | 300C11 | 46.50 |
| 60CB | 300C11 | 73.15 |
| 60CB | 300C11 | 87.59 |
| 60CB | 300C11 | 99.62 |
| 60CB | 300C11 | 31.95 |
| 60CB | 300C11 | 145.01 |
| 60CB | 300C11 | 57.96 |
| 60CB | 300C11 | 44.01 |
| 56O | 300C11 | 45.36 |
| 56O | 300C11 | 52.24 |
| 56O | 300C11 | 121.84 |
| 56O | 300C11 | 71.73 |
| 56O | 300C11 | 106.93 |
| 56O | 300C11 | 40.02 |
| 56O | 300C11 | 76.67 |
| 55O | 300C11 | 16.06 |
| 55O | 300C11 | 84.94 |
| 55O | 300C11 | 76.44 |
| 55O | 300C11 | 72.01 |
| 55O | 300C11 | 83.51 |
| 55O | 300C11 | 69.80 |
| 55C | 300C11 | 89.26 |
| 55C | 300C11 | 92.42 |
| 55C | 300C11 | 58.36 |
| 55C | 300C11 | 85.09 |
| 55C | 300C11 | 84.99 |
| 174CD2 | 300C11 | 68.13 |
| 174CD2 | 300C11 | 74.23 |
| 174CD2 | 300C11 | 157.02 |
| 174CD2 | 300C11 | 55.70 |
| 60CD | 300C11 | 132.31 |
| 60CD | 300C11 | 89.84 |
| 60CD | 300C11 | 14.49 |
| 95OG | 300C11 | 120.35 |
| 95OG | 300C11 | 118.05 |
| 62CB | 300C11 | 101.55 |
| 300I12 | 62CD1 | 3.514 |
| 300I12 | 67SG | 3.828 |
| 300I12 | 62CG | 3.904 |
| 300I12 | 62CE1 | 3.935 |
| 300I12 | 76CD2 | 3.948 |
| 300I12 | 219CZ3 | 4.003 |
| 300I12 | 219CE3 | 4.092 |
| 300I12 | 76CG | 4.264 |
| 300I12 | 62CB | 4.265 |
| 300I12 | 76NE2 | 4.337 |
| 300I12 | 62CD2 | 4.610 |
| 300I12 | 60CG | 4.623 |
| 300I12 | 62CZ | 4.643 |
| 300I12 | 56CB | 4.692 |
| 300I12 | 76CB | 4.770 |
| 300I12 | 76ND1 | 4.782 |
| 300I12 | 76CE1 | 4.820 |
| 300I12 | 60CB | 4.875 |
| 300I12 | 62CE2 | 4.945 |
| 300I12 | 219CH2 | 4.988 |
| 62CD1 | 300I12 | 82.42 |
| 62CD1 | 300I12 | 20.77 |
| 62CD1 | 300I12 | 20.60 |
| 62CD1 | 300I12 | 113.76 |
| 62CD1 | 300I12 | 61.51 |
| 62CD1 | 300I12 | 54.62 |
| 62CD1 | 300I12 | 105.54 |
| 62CD1 | 300I12 | 36.28 |
| 62CD1 | 300I12 | 132.00 |
| 62CD1 | 300I12 | 30.68 |
| 62CD1 | 300I12 | 103.00 |
| 62CD1 | 300I12 | 30.61 |
| 62CD1 | 300I12 | 74.90 |
| 62CD1 | 300I12 | 87.89 |
| 62CD1 | 300I12 | 117.42 |
| 62CD1 | 300I12 | 132.34 |
| 62CD1 | 300I12 | 85.12 |
| 62CD1 | 300I12 | 33.19 |
| 62CD1 | 300I12 | 55.86 |
| 67SG | 300I12 | 78.26 |
| 67SG | 300I12 | 68.36 |
| 67SG | 300I12 | 89.86 |
| 67SG | 300I12 | 138.28 |
| 67SG | 300I12 | 120.54 |
| 67SG | 300I12 | 72.01 |
| 67SG | 300I12 | 93.65 |
| 67SG | 300I12 | 90.83 |
| 67SG | 300I12 | 62.38 |
| 67SG | 300I12 | 143.21 |
| 67SG | 300I12 | 53.10 |
| 67SG | 300I12 | 70.74 |
| 67SG | 300I12 | 65.38 |
| 67SG | 300I12 | 65.19 |
| 67SG | 300I12 | 76.13 |
| 67SG | 300I12 | 143.71 |
| 67SG | 300I12 | 50.70 |
| 67SG | 300I12 | 137.57 |
| 62CG | 300I12 | 36.01 |
| 62CG | 300I12 | 133.44 |
| 62CG | 300I12 | 74.48 |
| 62CG | 300I12 | 72.59 |
| 62CG | 300I12 | 122.19 |
| 62CG | 300I12 | 20.71 |
| 62CG | 300I12 | 151.04 |
| 62CG | 300I12 | 16.29 |
| 62CG | 300I12 | 94.06 |
| 62CG | 300I12 | 37.02 |
| 62CG | 300I12 | 54.22 |
| 62CG | 300I12 | 104.22 |
| 62CG | 300I12 | 130.40 |
| 62CG | 300I12 | 146.21 |
| 62CG | 300I12 | 78.11 |
| 62CG | 300I12 | 28.90 |
| 62CG | 300I12 | 65.31 |
| 62CE1 | 300I12 | 97.74 |
| 62CE1 | 300I12 | 70.98 |
| 62CE1 | 300I12 | 57.53 |
| 62CE1 | 300I12 | 86.56 |
| 62CE1 | 300I12 | 55.22 |
| 62CE1 | 300I12 | 115.02 |
| 62CE1 | 300I12 | 36.83 |
| 62CE1 | 300I12 | 123.36 |
| 62CE1 | 300I12 | 16.13 |
| 62CE1 | 300I12 | 84.96 |
| 62CE1 | 300I12 | 68.61 |
| 62CE1 | 300I12 | 97.18 |
| 62CE1 | 300I12 | 112.53 |
| 62CE1 | 300I12 | 105.33 |
| 62CE1 | 300I12 | 28.66 |
| 62CE1 | 300I12 | 69.39 |
| 76CD2 | 300I12 | 86.59 |
| 76CD2 | 300I12 | 75.63 |
| 76CD2 | 300I12 | 18.49 |
| 76CD2 | 300I12 | 148.41 |
| 76CD2 | 300I12 | 18.34 |
| 76CD2 | 300I12 | 131.52 |
| 76CD2 | 300I12 | 119.26 |
| 76CD2 | 300I12 | 101.38 |
| 76CD2 | 300I12 | 158.00 |
| 76CD2 | 300I12 | 32.99 |
| 76CD2 | 300I12 | 26.87 |
| 76CD2 | 300I12 | 26.69 |
| 76CD2 | 300I12 | 126.27 |
| 76CD2 | 300I12 | 116.15 |
| 76CD2 | 300I12 | 99.45 |
| 219CZ3 | 300I12 | 19.81 |
| 219CZ3 | 300I12 | 96.82 |
| 219CZ3 | 300I12 | 70.12 |
| 219CZ3 | 300I12 | 98.01 |
| 219CZ3 | 300I12 | 89.88 |
| 219CZ3 | 300I12 | 70.31 |
| 219CZ3 | 300I12 | 86.96 |
| 219CZ3 | 300I12 | 114.71 |
| 219CZ3 | 300I12 | 91.47 |
| 219CZ3 | 300I12 | 111.91 |
| 219CZ3 | 300I12 | 112.14 |
| 219CZ3 | 300I12 | 57.90 |
| 219CZ3 | 300I12 | 94.53 |
| 219CZ3 | 300I12 | 12.86 |
| 219CE3 | 300I12 | 81.58 |
| 219CE3 | 300I12 | 75.60 |
| 219CE3 | 300I12 | 90.85 |
| 219CE3 | 300I12 | 85.27 |
| 219CE3 | 300I12 | 90.11 |
| 219CE3 | 300I12 | 73.51 |
| 219CE3 | 300I12 | 122.83 |
| 219CE3 | 300I12 | 73.24 |
| 219CE3 | 300I12 | 97.67 |
| 219CE3 | 300I12 | 102.32 |
| 219CE3 | 300I12 | 77.29 |
| 219CE3 | 300I12 | 84.76 |
| 219CE3 | 300I12 | 29.04 |
| 76CG | 300I12 | 141.68 |
| 76CG | 300I12 | 29.59 |
| 76CG | 300I12 | 116.01 |
| 76CG | 300I12 | 137.47 |
| 76CG | 300I12 | 86.56 |
| 76CG | 300I12 | 142.34 |
| 76CG | 300I12 | 17.98 |
| 76CG | 300I12 | 16.26 |
| 76CG | 300I12 | 27.22 |
| 76CG | 300I12 | 144.28 |
| 76CG | 300I12 | 99.91 |
| 76CG | 300I12 | 109.20 |
| 62CB | 300I12 | 166.16 |
| 62CB | 300I12 | 32.73 |
| 62CB | 300I12 | 73.49 |
| 62CB | 300I12 | 57.73 |
| 62CB | 300I12 | 47.23 |
| 62CB | 300I12 | 123.80 |
| 62CB | 300I12 | 151.11 |
| 62CB | 300I12 | 166.90 |
| 62CB | 300I12 | 58.21 |
| 62CB | 300I12 | 48.19 |
| 62CB | 300I12 | 58.26 |
| 76NE2 | 300I12 | 145.22 |
| 76NE2 | 300I12 | 109.94 |
| 76NE2 | 300I12 | 116.15 |
| 76NE2 | 300I12 | 146.21 |
| 76NE2 | 300I12 | 47.29 |
| 76NE2 | 300I12 | 26.61 |
| 76NE2 | 300I12 | 15.46 |
| 76NE2 | 300I12 | 122.14 |
| 76NE2 | 300I12 | 128.94 |
| 76NE2 | 300I12 | 110.44 |
| 62CD2 | 300I12 | 104.63 |
| 62CD2 | 300I12 | 30.15 |
| 62CD2 | 300I12 | 48.14 |
| 62CD2 | 300I12 | 98.98 |
| 62CD2 | 300I12 | 119.90 |
| 62CD2 | 300I12 | 134.86 |
| 62CD2 | 300I12 | 90.70 |
| 62CD2 | 300I12 | 16.30 |
| 62CD2 | 300I12 | 81.38 |
| 60CG | 300I12 | 130.86 |
| 60CG | 300I12 | 75.63 |
| 60CG | 300I12 | 149.83 |
| 60CG | 300I12 | 135.21 |
| 60CG | 300I12 | 119.59 |
| 60CG | 300I12 | 18.10 |
| 60CG | 300I12 | 120.89 |
| 60CG | 300I12 | 63.77 |
| 62CZ | 300I12 | 75.64 |
| 62CZ | 300I12 | 69.07 |
| 62CZ | 300I12 | 93.38 |
| 62CZ | 300I12 | 109.19 |
| 62CZ | 300I12 | 113.99 |
| 62CZ | 300I12 | 16.25 |
| 62CZ | 300I12 | 84.48 |
| 56CB | 300I12 | 134.53 |
| 56CB | 300I12 | 131.18 |
| 56CB | 300I12 | 133.10 |
| 56CB | 300I12 | 73.12 |
| 56CB | 300I12 | 59.40 |
| 56CB | 300I12 | 101.91 |
| 76CB | 300I12 | 30.65 |
| 76CB | 300I12 | 44.50 |
| 76CB | 300I12 | 147.98 |
| 76CB | 300I12 | 83.25 |
| 76CB | 300I12 | 102.26 |
| 76ND1 | 300I12 | 15.82 |
| 76ND1 | 300I12 | 148.74 |
| 76ND1 | 300I12 | 103.77 |
| 76ND1 | 300I12 | 124.59 |
| 76CE1 | 300I12 | 134.45 |
| 76CE1 | 300I12 | 119.02 |
| 76CE1 | 300I12 | 124.87 |
| 60CB | 300I12 | 106.40 |
| 60CB | 300I12 | 48.92 |
| 62CE2 | 300I12 | 88.84 |
| 253ZN | 104OD2 | 2.002 |
| 253ZN | 168NE2 | 2.077 |
| 253ZN | 233OE2 | 2.478 |
| 253ZN | 202OE1 | 2.724 |
| 253ZN | 168CE1 | 3.012 |
| 253ZN | 104CG | 3.089 |
| 253ZN | 168CD2 | 3.090 |
| 253ZN | 202CD | 3.412 |
| 253ZN | 104OD1 | 3.542 |
| 253ZN | 233CD | 3.600 |
| 253ZN | 202OE2 | 3.676 |
| 253ZN | 233OE1 | 4.064 |
| 253ZN | 168ND1 | 4.134 |
| 253ZN | 168CG | 4.202 |
| 253ZN | 104CB | 4.344 |
| 253ZN | 202CG | 4.573 |
| 253ZN | 175CE1 | 4.626 |
| 253ZN | 233CG | 4.836 |
| 253ZN | 174CD1 | 4.849 |
| 253ZN | 93OD2 | 4.962 |
| 104OD2 | 253ZN | 94.40 |
| 104OD2 | 253ZN | 74.39 |
| 104OD2 | 253ZN | 143.03 |
| 104OD2 | 253ZN | 73.00 |
| 104OD2 | 253ZN | 14.18 |
| 104OD2 | 253ZN | 114.10 |
| 104OD2 | 253ZN | 131.83 |
| 104OD2 | 253ZN | 34.22 |
| 104OD2 | 253ZN | 71.69 |
| 104OD2 | 253ZN | 129.77 |
| 104OD2 | 253ZN | 72.33 |
| 104OD2 | 253ZN | 83.25 |
| 104OD2 | 253ZN | 101.83 |
| 104OD2 | 253ZN | 4.87 |
| 104OD2 | 253ZN | 120.51 |
| 104OD2 | 253ZN | 107.34 |
| 104OD2 | 253ZN | 70.68 |
| 104OD2 | 253ZN | 57.15 |
| 104OD2 | 253ZN | 90.69 |
| 168NE2 | 253ZN | 131.44 |
| 168NE2 | 253ZN | 88.08 |
| 168NE2 | 253ZN | 21.44 |
| 168NE2 | 253ZN | 108.51 |
| 168NE2 | 253ZN | 21.13 |
| 168NE2 | 253ZN | 106.27 |
| 168NE2 | 253ZN | 127.51 |
| 168NE2 | 253ZN | 140.75 |
| 168NE2 | 253ZN | 124.02 |
| 168NE2 | 253ZN | 157.03 |
| 168NE2 | 253ZN | 11.72 |
| 168NE2 | 253ZN | 10.72 |
| 168NE2 | 253ZN | 96.50 |
| 168NE2 | 253ZN | 101.04 |
| 168NE2 | 253ZN | 46.01 |
| 168NE2 | 253ZN | 128.77 |
| 168NE2 | 253ZN | 72.14 |
| 168NE2 | 253ZN | 169.34 |
| 233OE2 | 253ZN | 76.50 |
| 233OE2 | 253ZN | 123.58 |
| 233OE2 | 253ZN | 66.35 |
| 233OE2 | 253ZN | 126.95 |
| 233OE2 | 253ZN | 59.18 |
| 233OE2 | 253ZN | 61.23 |
| 233OE2 | 253ZN | 10.43 |
| 233OE2 | 253ZN | 56.28 |
| 233OE2 | 253ZN | 27.78 |
| 233OE2 | 253ZN | 124.11 |
| 233OE2 | 253ZN | 125.70 |
| 233OE2 | 253ZN | 69.71 |
| 233OE2 | 253ZN | 52.63 |
| 233OE2 | 253ZN | 176.65 |
| 233OE2 | 253ZN | 4.26 |
| 233OE2 | 253ZN | 128.78 |
| 233OE2 | 253ZN | 59.04 |
| 202OE1 | 253ZN | 105.92 |
| 202OE1 | 253ZN | 141.42 |
| 202OE1 | 253ZN | 67.86 |
| 202OE1 | 253ZN | 19.49 |
| 202OE1 | 253ZN | 136.35 |
| 202OE1 | 253ZN | 83.25 |
| 202OE1 | 253ZN | 36.04 |
| 202OE1 | 253ZN | 91.70 |
| 202OE1 | 253ZN | 94.81 |
| 202OE1 | 253ZN | 77.48 |
| 202OE1 | 253ZN | 138.51 |
| 202OE1 | 253ZN | 24.41 |
| 202OE1 | 253ZN | 100.77 |
| 202OE1 | 253ZN | 79.13 |
| 202OE1 | 253ZN | 154.44 |
| 202OE1 | 253ZN | 93.50 |
| 168CE1 | 253ZN | 87.09 |
| 168CE1 | 253ZN | 42.10 |
| 168CE1 | 253ZN | 121.90 |
| 168CE1 | 253ZN | 106.15 |
| 168CE1 | 253ZN | 129.88 |
| 168CE1 | 253ZN | 141.41 |
| 168CE1 | 253ZN | 141.19 |
| 168CE1 | 253ZN | 11.37 |
| 168CE1 | 253ZN | 30.24 |
| 168CE1 | 253ZN | 75.27 |
| 168CE1 | 253ZN | 112.89 |
| 168CE1 | 253ZN | 55.05 |
| 168CE1 | 253ZN | 119.78 |
| 168CE1 | 253ZN | 59.29 |
| 168CE1 | 253ZN | 160.53 |
| 104CG | 253ZN | 128.27 |
| 104CG | 253ZN | 125.52 |
| 104CG | 253ZN | 20.26 |
| 104CG | 253ZN | 61.66 |
| 104CG | 253ZN | 118.83 |
| 104CG | 253ZN | 59.57 |
| 104CG | 253ZN | 97.42 |
| 104CG | 253ZN | 116.00 |
| 104CG | 253ZN | 13.28 |
| 104CG | 253ZN | 117.07 |
| 104CG | 253ZN | 115.95 |
| 104CG | 253ZN | 63.20 |
| 104CG | 253ZN | 62.71 |
| 104CG | 253ZN | 76.54 |
| 168CD2 | 253ZN | 86.80 |
| 168CD2 | 253ZN | 148.07 |
| 168CD2 | 253ZN | 137.38 |
| 168CD2 | 253ZN | 103.40 |
| 168CD2 | 253ZN | 153.94 |
| 168CD2 | 253ZN | 31.14 |
| 168CD2 | 253ZN | 12.27 |
| 168CD2 | 253ZN | 115.51 |
| 168CD2 | 253ZN | 84.09 |
| 168CD2 | 253ZN | 49.79 |
| 168CD2 | 253ZN | 126.06 |
| 168CD2 | 253ZN | 90.11 |
| 168CD2 | 253ZN | 155.17 |
| 202CD | 253ZN | 117.15 |
| 202CD | 253ZN | 64.75 |
| 202CD | 253ZN | 19.78 |
| 202CD | 253ZN | 72.22 |
| 202CD | 253ZN | 111.47 |
| 202CD | 253ZN | 95.56 |
| 202CD | 253ZN | 126.96 |
| 202CD | 253ZN | 14.32 |
| 202CD | 253ZN | 118.43 |
| 202CD | 253ZN | 62.36 |
| 202CD | 253ZN | 170.90 |
| 202CD | 253ZN | 76.87 |
| 104OD1 | 253ZN | 53.39 |
| 104OD1 | 253ZN | 104.82 |
| 104OD1 | 253ZN | 45.35 |
| 104OD1 | 253ZN | 116.95 |
| 104OD1 | 253ZN | 135.84 |
| 104OD1 | 253ZN | 33.47 |
| 104OD1 | 253ZN | 113.66 |
| 104OD1 | 253ZN | 121.81 |
| 104OD1 | 253ZN | 59.41 |
| 104OD1 | 253ZN | 69.10 |
| 104OD1 | 253ZN | 56.56 |
| 233CD | 253ZN | 58.09 |
| 233CD | 253ZN | 17.51 |
| 233CD | 253ZN | 132.27 |
| 233CD | 253ZN | 135.82 |
| 233CD | 253ZN | 67.35 |
| 233CD | 253ZN | 60.35 |
| 233CD | 253ZN | 172.75 |
| 233CD | 253ZN | 12.10 |
| 233CD | 253ZN | 122.31 |
| 233CD | 253ZN | 49.89 |
| 202OE2 | 253ZN | 59.91 |
| 202OE2 | 253ZN | 130.61 |
| 202OE2 | 253ZN | 113.49 |
| 202OE2 | 253ZN | 125.40 |
| 202OE2 | 253ZN | 31.41 |
| 202OE2 | 253ZN | 122.43 |
| 202OE2 | 253ZN | 60.31 |
| 202OE2 | 253ZN | 156.50 |
| 202OE2 | 253ZN | 57.74 |
| 233OE1 | 253ZN | 146.80 |
| 233OE1 | 253ZN | 153.31 |
| 233OE1 | 253ZN | 68.93 |
| 233OE1 | 253ZN | 71.73 |
| 233OE1 | 253ZN | 155.26 |
| 233OE1 | 253ZN | 29.55 |
| 233OE1 | 253ZN | 112.87 |
| 233OE1 | 253ZN | 33.55 |
| 168ND1 | 253ZN | 19.04 |
| 168ND1 | 253ZN | 85.10 |
| 168ND1 | 253ZN | 103.69 |
| 168ND1 | 253ZN | 53.83 |
| 168ND1 | 253ZN | 120.85 |
| 168ND1 | 253ZN | 68.77 |
| 168ND1 | 253ZN | 171.64 |
| 168CG | 253ZN | 103.29 |
| 168CG | 253ZN | 90.55 |
| 168CG | 253ZN | 51.36 |
| 168CG | 253ZN | 123.76 |
| 168CG | 253ZN | 82.76 |
| 168CG | 253ZN | 167.39 |
| 104CB | 253ZN | 115.71 |
| 104CB | 253ZN | 111.94 |
| 104CB | 253ZN | 65.96 |
| 104CB | 253ZN | 62.01 |
| 104CB | 253ZN | 89.31 |
| 202CG | 253ZN | 124.45 |
| 202CG | 253ZN | 54.97 |
| 202CG | 253ZN | 171.98 |
| 202CG | 253ZN | 84.33 |
| 175CE1 | 253ZN | 174.69 |
| 175CE1 | 253ZN | 53.82 |
| 175CE1 | 253ZN | 123.42 |
| 233CG | 253ZN | 125.86 |
| 233CG | 253ZN | 61.82 |
| 174CD1 | 253ZN | 103.10 |
| 254ZN | 93OD1 | 1.948 |
| 254ZN | 233OE1 | 2.009 |
| 254ZN | 104OD1 | 2.039 |
| 254ZN | 93OD2 | 2.234 |
| 254ZN | 93CG | 2.383 |
| 254ZN | 233CD | 2.777 |
| 254ZN | 233OE2 | 2.932 |
| 254ZN | 104CG | 2.977 |
| 254ZN | 104OD2 | 3.259 |
| 254ZN | 93CB | 3.899 |
| 254ZN | 233CG | 4.190 |
| 254ZN | 104CB | 4.361 |
| 254ZN | 202OE2 | 4.418 |
| 254ZN | 93CA | 4.651 |
| 254ZN | 104C | 4.653 |
| 254ZN | 104CA | 4.729 |
| 254ZN | 95CB | 4.855 |
| 254ZN | 93C | 4.886 |
| 254ZN | 233CB | 4.893 |
| 93OD1 | 254ZN | 105.84 |
| 93OD1 | 254ZN | 102.39 |
| 93OD1 | 254ZN | 62.92 |
| 93OD1 | 254ZN | 31.47 |
| 93OD1 | 254ZN | 128.84 |
| 93OD1 | 254ZN | 153.84 |
| 93OD1 | 254ZN | 121.26 |
| 93OD1 | 254ZN | 140.80 |
| 93OD1 | 254ZN | 28.71 |
| 93OD1 | 254ZN | 120.57 |
| 93OD1 | 254ZN | 113.59 |
| 93OD1 | 254ZN | 135.56 |
| 93OD1 | 254ZN | 20.63 |
| 93OD1 | 254ZN | 96.27 |
| 93OD1 | 254ZN | 97.12 |
| 93OD1 | 254ZN | 69.85 |
| 93OD1 | 254ZN | 6.97 |
| 93OD1 | 254ZN | 108.86 |
| 233OE1 | 254ZN | 94.44 |
| 233OE1 | 254ZN | 80.38 |
| 233OE1 | 254ZN | 93.64 |
| 233OE1 | 254ZN | 24.22 |
| 233OE1 | 254ZN | 48.56 |
| 233OE1 | 254ZN | 92.12 |
| 233OE1 | 254ZN | 94.04 |
| 233OE1 | 254ZN | 94.96 |
| 233OE1 | 254ZN | 19.36 |
| 233OE1 | 254ZN | 88.80 |
| 233OE1 | 254ZN | 61.38 |
| 233OE1 | 254ZN | 87.96 |
| 233OE1 | 254ZN | 66.56 |
| 233OE1 | 254ZN | 84.36 |
| 233OE1 | 254ZN | 131.39 |
| 233OE1 | 254ZN | 99.74 |
| 233OE1 | 254ZN | 3.02 |
| 104OD1 | 254ZN | 161.74 |
| 104OD1 | 254ZN | 133.07 |
| 104OD1 | 254ZN | 82.02 |
| 104OD1 | 254ZN | 77.86 |
| 104OD1 | 254ZN | 19.27 |
| 104OD1 | 254ZN | 41.51 |
| 104OD1 | 254ZN | 130.44 |
| 104OD1 | 254ZN | 79.14 |
| 104OD1 | 254ZN | 11.46 |
| 104OD1 | 254ZN | 120.14 |
| 104OD1 | 254ZN | 114.72 |
| 104OD1 | 254ZN | 31.12 |
| 104OD1 | 254ZN | 13.12 |
| 104OD1 | 254ZN | 44.32 |
| 104OD1 | 254ZN | 99.81 |
| 104OD1 | 254ZN | 93.51 |
| 93OD2 | 254ZN | 31.45 |
| 93OD2 | 254ZN | 98.64 |
| 93OD2 | 254ZN | 110.23 |
| 93OD2 | 254ZN | 172.35 |
| 93OD2 | 254ZN | 155.56 |
| 93OD2 | 254ZN | 34.22 |
| 93OD2 | 254ZN | 98.59 |
| 93OD2 | 254ZN | 166.77 |
| 93OD2 | 254ZN | 72.78 |
| 93OD2 | 254ZN | 48.01 |
| 93OD2 | 254ZN | 134.59 |
| 93OD2 | 254ZN | 149.89 |
| 93OD2 | 254ZN | 129.30 |
| 93OD2 | 254ZN | 64.27 |
| 93OD2 | 254ZN | 82.14 |
| 93CG | 254ZN | 117.19 |
| 93CG | 254ZN | 136.84 |
| 93CG | 254ZN | 152.30 |
| 93CG | 254ZN | 171.03 |
| 93CG | 254ZN | 2.77 |
| 93CG | 254ZN | 112.77 |
| 93CG | 254ZN | 143.67 |
| 93CG | 254ZN | 104.18 |
| 93CG | 254ZN | 19.90 |
| 93CG | 254ZN | 118.47 |
| 93CG | 254ZN | 125.48 |
| 93CG | 254ZN | 99.73 |
| 93CG | 254ZN | 33.28 |
| 93CG | 254ZN | 96.36 |
| 233CD | 254ZN | 25.03 |
| 233CD | 254ZN | 73.74 |
| 233CD | 254ZN | 70.98 |
| 233CD | 254ZN | 118.73 |
| 233CD | 254ZN | 9.27 |
| 233CD | 254ZN | 73.33 |
| 233CD | 254ZN | 53.06 |
| 233CD | 254ZN | 112.04 |
| 233CD | 254ZN | 61.95 |
| 233CD | 254ZN | 75.81 |
| 233CD | 254ZN | 125.78 |
| 233CD | 254ZN | 122.28 |
| 233CD | 254ZN | 21.28 |
| 233OE2 | 254ZN | 63.04 |
| 233OE2 | 254ZN | 52.10 |
| 233OE2 | 254ZN | 139.06 |
| 233OE2 | 254ZN | 33.32 |
| 233OE2 | 254ZN | 66.81 |
| 233OE2 | 254ZN | 44.20 |
| 233OE2 | 254ZN | 136.36 |
| 233OE2 | 254ZN | 70.21 |
| 233OE2 | 254ZN | 77.23 |
| 233OE2 | 254ZN | 120.33 |
| 233OE2 | 254ZN | 147.17 |
| 233OE2 | 254ZN | 45.54 |
| 104CG | 254ZN | 22.51 |
| 104CG | 254ZN | 149.64 |
| 104CG | 254ZN | 73.79 |
| 104CG | 254ZN | 9.75 |
| 104CG | 254ZN | 102.36 |
| 104CG | 254ZN | 133.83 |
| 104CG | 254ZN | 41.30 |
| 104CG | 254ZN | 28.60 |
| 104CG | 254ZN | 57.30 |
| 104CG | 254ZN | 119.02 |
| 104CG | 254ZN | 90.30 |
| 104OD2 | 254ZN | 168.55 |
| 104OD2 | 254ZN | 74.72 |
| 104OD2 | 254ZN | 32.05 |
| 104OD2 | 254ZN | 83.62 |
| 104OD2 | 254ZN | 156.22 |
| 104OD2 | 254ZN | 60.90 |
| 104OD2 | 254ZN | 50.89 |
| 104OD2 | 254ZN | 71.64 |
| 104OD2 | 254ZN | 140.08 |
| 104OD2 | 254ZN | 91.44 |
| 93CB | 254ZN | 113.94 |
| 93CB | 254ZN | 141.16 |
| 93CB | 254ZN | 106.95 |
| 93CB | 254ZN | 18.04 |
| 93CB | 254ZN | 116.79 |
| 93CB | 254ZN | 123.14 |
| 93CB | 254ZN | 97.05 |
| 93CB | 254ZN | 30.63 |
| 93CB | 254ZN | 97.73 |
| 233CG | 254ZN | 71.76 |
| 233CG | 254ZN | 61.28 |
| 233CG | 254ZN | 105.07 |
| 233CG | 254ZN | 55.74 |
| 233CG | 254ZN | 71.25 |
| 233CG | 254ZN | 121.27 |
| 233CG | 254ZN | 113.84 |
| 233CG | 254ZN | 16.95 |
| 104CB | 254ZN | 108.68 |
| 104CB | 254ZN | 124.55 |
| 104CB | 254ZN | 32.31 |
| 104CB | 254ZN | 18.89 |
| 104CB | 254ZN | 54.38 |
| 104CB | 254ZN | 110.65 |
| 104CB | 254ZN | 87.40 |
| 202OE2 | 254ZN | 117.65 |
| 202OE2 | 254ZN | 113.05 |
| 202OE2 | 254ZN | 121.35 |
| 202OE2 | 254ZN | 152.12 |
| 202OE2 | 254ZN | 135.66 |
| 202OE2 | 254ZN | 59.45 |
| 93CA | 254ZN | 98.76 |
| 93CA | 254ZN | 105.94 |
| 93CA | 254ZN | 89.31 |
| 93CA | 254ZN | 18.16 |
| 93CA | 254ZN | 90.97 |
| 104C | 254ZN | 18.64 |
| 104C | 254ZN | 65.97 |
| 104C | 254ZN | 90.76 |
| 104C | 254ZN | 66.41 |
| 104CA | 254ZN | 50.13 |
| 104CA | 254ZN | 93.27 |
| 104CA | 254ZN | 83.88 |
| 95CB | 254ZN | 71.15 |
| 95CB | 254ZN | 131.83 |
| 93C | 254ZN | 102.74 |

TABLE V
Provides distances between interresidue atoms that are within 5 Ångstroms
apart in an active site of a S. aureus methionine aminopeptidase
for an inhibitor complex with 5-benzofuran-2-yl-1-H-[1,2,3]triazole.

| Atom 1 | Atom 2 | Distance |
|---|---|---|
| 300C1 | 76CE1 | 3.780 |
| 300C1 | 76NE2 | 4.027 |
| 300C1 | 174CD2 | 4.064 |
| 300C1 | 175CE1 | 4.370 |
| 300C1 | 76ND1 | 4.454 |
| 300C1 | 76CD2 | 4.830 |
| 300C1 | 175NE2 | 4.920 |
| 300C1 | 174CG | 4.960 |
| 300C1 | 67SG | 4.988 |
| 76CE1 | 300C1 | 19.14 |
| 76CE1 | 300C1 | 164.38 |
| 76CE1 | 300C1 | 113.69 |
| 76CE1 | 300C1 | 15.95 |
| 76CE1 | 300C1 | 26.06 |
| 76CE1 | 300C1 | 98.82 |
| 76CE1 | 300C1 | 155.51 |
| 76CE1 | 300C1 | 74.42 |
| 76NE2 | 300C1 | 147.39 |
| 76NE2 | 300C1 | 105.69 |
| 76NE2 | 300C1 | 28.72 |
| 76NE2 | 300C1 | 14.53 |
| 76NE2 | 300C1 | 91.86 |
| 76NE2 | 300C1 | 146.70 |
| 76NE2 | 300C1 | 81.18 |
| 174CD2 | 300C1 | 52.87 |
| 174CD2 | 300C1 | 172.05 |
| 174CD2 | 300C1 | 146.40 |
| 174CD2 | 300C1 | 67.72 |
| 174CD2 | 300C1 | 15.79 |
| 174CD2 | 300C1 | 118.71 |
| 175CE1 | 300C1 | 129.65 |
| 175CE1 | 300C1 | 116.73 |
| 175CE1 | 300C1 | 14.91 |
| 175CE1 | 300C1 | 42.43 |
| 175CE1 | 300C1 | 171.48 |
| 76ND1 | 300C1 | 26.83 |
| 76ND1 | 300C1 | 114.78 |
| 76ND1 | 300C1 | 170.79 |
| 76ND1 | 300C1 | 58.47 |
| 76CD2 | 300C1 | 103.95 |
| 76CD2 | 300C1 | 153.05 |
| 76CD2 | 300C1 | 69.24 |
| 175NE2 | 300C1 | 57.13 |
| 175NE2 | 300C1 | 173.03 |
| 174CG | 300C1 | 129.75 |
| 300C2 | 174CD2 | 4.134 |
| 300C2 | 76CE1 | 4.368 |
| 300C2 | 175CE1 | 4.430 |
| 300C2 | 93OD2 | 4.624 |
| 300C2 | 93OD1 | 4.702 |
| 300C2 | 174CG | 4.705 |
| 300C2 | 104OD2 | 4.723 |
| 300C2 | 174CD1 | 4.797 |
| 300C2 | 93CG | 4.837 |
| 300C2 | 95OG | 4.856 |
| 300C2 | 104OD1 | 4.899 |
| 300C2 | 76NE2 | 4.938 |
| 174CD2 | 300C2 | 132.15 |
| 174CD2 | 300C2 | 52.02 |
| 174CD2 | 300C2 | 139.66 |
| 174CD2 | 300C2 | 113.90 |
| 174CD2 | 300C2 | 18.44 |
| 174CD2 | 300C2 | 80.51 |
| 174CD2 | 300C2 | 31.45 |
| 174CD2 | 300C2 | 128.63 |
| 174CD2 | 300C2 | 55.04 |
| 174CD2 | 300C2 | 88.85 |
| 174CD2 | 300C2 | 117.47 |
| 76CE1 | 300C2 | 101.87 |
| 76CE1 | 300C2 | 87.27 |
| 76CE1 | 300C2 | 109.71 |
| 76CE1 | 300C2 | 140.71 |
| 76CE1 | 300C2 | 138.19 |
| 76CE1 | 300C2 | 158.66 |
| 76CE1 | 300C2 | 95.03 |
| 76CE1 | 300C2 | 146.77 |
| 76CE1 | 300C2 | 138.79 |
| 76CE1 | 300C2 | 14.74 |
| 175CE1 | 300C2 | 140.03 |
| 175CE1 | 300C2 | 140.77 |
| 175CE1 | 300C2 | 43.86 |
| 175CE1 | 300C2 | 77.13 |
| 175CE1 | 300C2 | 57.79 |
| 175CE1 | 300C2 | 148.67 |
| 175CE1 | 300C2 | 102.80 |
| 175CE1 | 300C2 | 102.01 |
| 175CE1 | 300C2 | 91.07 |
| 93OD2 | 300C2 | 27.20 |
| 93OD2 | 300C2 | 130.52 |
| 93OD2 | 300C2 | 70.80 |
| 93OD2 | 300C2 | 112.31 |
| 93OD2 | 300C2 | 14.93 |
| 93OD2 | 300C2 | 87.95 |
| 93OD2 | 300C2 | 53.47 |
| 93OD2 | 300C2 | 101.97 |
| 93OD1 | 300C2 | 109.58 |
| 93OD1 | 300C2 | 63.87 |
| 93OD1 | 300C2 | 91.49 |
| 93OD1 | 300C2 | 14.94 |
| 93OD1 | 300C2 | 60.76 |
| 93OD1 | 300C2 | 38.83 |
| 93OD1 | 300C2 | 123.45 |
| 174CG | 300C2 | 63.86 |
| 174CG | 300C2 | 18.41 |
| 174CG | 300C2 | 124.25 |
| 174CG | 300C2 | 58.94 |
| 174CG | 300C2 | 77.06 |
| 174CG | 300C2 | 126.57 |
| 104OD2 | 300C2 | 49.42 |
| 104OD2 | 300C2 | 72.72 |
| 104OD2 | 300C2 | 69.61 |
| 104OD2 | 300C2 | 26.43 |
| 104OD2 | 300C2 | 145.50 |
| 174CD1 | 300C2 | 105.98 |
| 174CD1 | 300C2 | 47.37 |
| 174CD1 | 300C2 | 59.00 |
| 174CD1 | 300C2 | 144.92 |
| 93CG | 300C2 | 74.56 |
| 93CG | 300C2 | 50.31 |
| 93CG | 300C2 | 109.09 |
| 95OG | 300C2 | 54.84 |
| 95OG | 300C2 | 144.86 |
| 104OD1 | 300C2 | 153.06 |
| 300C3 | 76CE1 | 3.808 |
| 300C3 | 67SG | 3.904 |
| 300C3 | 76NE2 | 3.933 |
| 300C3 | 76ND1 | 4.133 |
| 300C3 | 76CD2 | 4.345 |
| 300C3 | 76CG | 4.470 |
| 300C3 | 174CD2 | 4.637 |
| 300C3 | 56CG | 4.786 |
| 76CE1 | 300C3 | 88.64 |
| 76CE1 | 300C3 | 19.56 |
| 76CE1 | 300C3 | 18.60 |
| 76CE1 | 300C3 | 30.32 |
| 76CE1 | 300C3 | 29.53 |
| 76CE1 | 300C3 | 133.72 |
| 76CE1 | 300C3 | 151.39 |
| 67SG | 300C3 | 97.91 |
| 67SG | 300C3 | 70.36 |
| 67SG | 300C3 | 84.94 |
| 67SG | 300C3 | 68.93 |
| 67SG | 300C3 | 131.79 |
| 67SG | 300C3 | 63.16 |
| 76NE2 | 300C3 | 30.70 |
| 76NE2 | 300C3 | 18.26 |
| 76NE2 | 300C3 | 29.44 |
| 76NE2 | 300C3 | 129.79 |
| 76NE2 | 300C3 | 157.76 |
| 76ND1 | 300C3 | 29.74 |
| 76ND1 | 300C3 | 17.88 |
| 76ND1 | 300C3 | 151.31 |
| 76ND1 | 300C3 | 133.50 |
| 76CD2 | 300C3 | 17.62 |
| 76CD2 | 300C3 | 142.99 |
| 76CD2 | 300C3 | 139.86 |
| 76CG | 300C3 | 159.17 |
| 76CG | 300C3 | 129.15 |
| 174CD2 | 300C3 | 71.10 |
| 300O4 | 175CE1 | 3.522 |
| 300O4 | 76NE2 | 3.714 |
| 300O4 | 76CE1 | 3.846 |
| 300O4 | 175NE2 | 3.910 |
| 300O4 | 174CD2 | 4.110 |
| 300O4 | 76CD2 | 4.656 |
| 300O4 | 175ND1 | 4.663 |
| 300O4 | 76ND1 | 4.790 |
| 300O4 | 174CG | 4.910 |
| 175CE1 | 300O4 | 135.39 |
| 175CE1 | 300O4 | 135.94 |
| 175CE1 | 300O4 | 19.62 |
| 175CE1 | 300O4 | 58.51 |
| 175CE1 | 300O4 | 146.44 |
| 175CE1 | 300O4 | 9.44 |
| 175CE1 | 300O4 | 147.36 |
| 175CE1 | 300O4 | 44.17 |
| 76NE2 | 300O4 | 20.02 |
| 76NE2 | 300O4 | 115.77 |
| 76NE2 | 300O4 | 165.92 |
| 76NE2 | 300O4 | 13.84 |
| 76NE2 | 300O4 | 141.55 |
| 76NE2 | 300O4 | 25.39 |
| 76NE2 | 300O4 | 174.40 |
| 76CE1 | 300O4 | 118.02 |
| 76CE1 | 300O4 | 155.30 |
| 76CE1 | 300O4 | 27.89 |
| 76CE1 | 300O4 | 144.90 |
| 76CE1 | 300O4 | 12.39 |
| 76CE1 | 300O4 | 154.63 |
| 175NE2 | 300O4 | 78.13 |
| 175NE2 | 300O4 | 127.25 |
| 175NE2 | 300O4 | 27.17 |
| 175NE2 | 300O4 | 130.20 |
| 175NE2 | 300O4 | 63.60 |
| 174CD2 | 300O4 | 152.62 |
| 174CD2 | 300O4 | 51.81 |
| 174CD2 | 300O4 | 145.29 |
| 174CD2 | 300O4 | 16.61 |
| 76CD2 | 300O4 | 150.34 |
| 76CD2 | 300O4 | 26.70 |
| 76CD2 | 300O4 | 168.88 |
| 175ND1 | 300O4 | 156.65 |
| 175ND1 | 300O4 | 39.13 |
| 76ND1 | 300O4 | 150.63 |
| 300C5 | 93OD1 | 3.375 |
| 300C5 | 93CG | 3.623 |
| 300C5 | 93OD2 | 3.631 |
| 300C5 | 104OD1 | 3.988 |
| 300C5 | 95OG | 4.148 |
| 300C5 | 104OD2 | 4.399 |
| 300C5 | 104CG | 4.545 |
| 300C5 | 93CB | 4.628 |
| 300C5 | 95CB | 4.691 |
| 300C5 | 174CD2 | 4.695 |
| 300C5 | 233OE1 | 4.735 |
| 300C5 | 174CD1 | 4.925 |
| 300C5 | 67SG | 4.989 |
| 93OD1 | 300C5 | 20.13 |
| 93OD1 | 300C5 | 36.26 |
| 93OD1 | 300C5 | 50.64 |
| 93OD1 | 300C5 | 79.27 |
| 93OD1 | 300C5 | 78.56 |
| 93OD1 | 300C5 | 65.61 |
| 93OD1 | 300C5 | 29.58 |
| 93OD1 | 300C5 | 69.09 |
| 93OD1 | 300C5 | 132.75 |
| 93OD1 | 300C5 | 43.84 |
| 93OD1 | 300C5 | 108.66 |
| 93OD1 | 300C5 | 88.44 |
| 93CG | 300C5 | 19.79 |
| 93CG | 300C5 | 65.68 |
| 93CG | 300C5 | 97.90 |
| 93CG | 300C5 | 89.37 |
| 93CG | 300C5 | 79.56 |
| 93CG | 300C5 | 15.86 |
| 93CG | 300C5 | 89.03 |
| 93CG | 300C5 | 152.83 |
| 93CG | 300C5 | 44.29 |
| 93CG | 300C5 | 127.62 |
| 93CG | 300C5 | 79.99 |
| 93OD2 | 300C5 | 68.37 |
| 93OD2 | 300C5 | 115.49 |
| 93OD2 | 300C5 | 84.21 |
| 93OD2 | 300C5 | 79.26 |
| 93OD2 | 300C5 | 30.45 |
| 93OD2 | 300C5 | 103.93 |
| 93OD2 | 300C5 | 161.69 |
| 93OD2 | 300C5 | 35.41 |
| 93OD2 | 300C5 | 131.66 |
| 93OD2 | 300C5 | 88.68 |
| 104OD1 | 300C5 | 67.01 |
| 104OD1 | 300C5 | 30.00 |
| 104OD1 | 300C5 | 15.11 |
| 104OD1 | 300C5 | 79.44 |
| 104OD1 | 300C5 | 50.02 |
| 104OD1 | 300C5 | 93.57 |
| 104OD1 | 300C5 | 38.72 |
| 104OD1 | 300C5 | 63.77 |
| 104OD1 | 300C5 | 135.84 |
| 95OG | 300C5 | 79.50 |
| 95OG | 300C5 | 68.75 |
| 95OG | 300C5 | 97.18 |
| 95OG | 300C5 | 16.99 |
| 95OG | 300C5 | 56.35 |
| 95OG | 300C5 | 103.03 |
| 95OG | 300C5 | 49.70 |
| 95OG | 300C5 | 92.59 |
| 104OD2 | 300C5 | 16.02 |
| 104OD2 | 300C5 | 104.86 |
| 104OD2 | 300C5 | 64.77 |
| 104OD2 | 300C5 | 78.21 |
| 104OD2 | 300C5 | 48.81 |
| 104OD2 | 300C5 | 50.15 |
| 104OD2 | 300C5 | 165.81 |
| 104CG | 300C5 | 93.97 |
| 104CG | 300C5 | 52.56 |
| 104CG | 300C5 | 82.43 |
| 104CG | 300C5 | 45.66 |
| 104CG | 300C5 | 52.45 |
| 104CG | 300C5 | 149.92 |
| 93CB | 300C5 | 92.58 |
| 93CB | 300C5 | 152.83 |
| 93CB | 300C5 | 59.99 |
| 93CB | 300C5 | 137.20 |
| 93CB | 300C5 | 64.15 |
| 95CB | 300C5 | 63.84 |
| 95CB | 300C5 | 86.55 |
| 95CB | 300C5 | 47.31 |
| 95CB | 300C5 | 105.36 |
| 174CD2 | 300C5 | 126.85 |
| 174CD2 | 300C5 | 30.04 |
| 174CD2 | 300C5 | 107.31 |
| 233OE1 | 300C5 | 97.53 |
| 233OE1 | 300C5 | 123.27 |
| 174CD1 | 300C5 | 131.42 |
| 300N6 | 168NE2 | 3.672 |
| 300N6 | 175CE1 | 3.765 |
| 300N6 | 104OD2 | 3.797 |
| 300N6 | 175NE2 | 4.231 |
| 300N6 | 174CD2 | 4.332 |
| 300N6 | 168CE1 | 4.440 |
| 300N6 | 174CG | 4.460 |
| 300N6 | 174CD1 | 4.485 |
| 300N6 | 104OD1 | 4.502 |
| 300N6 | 202OE1 | 4.508 |
| 300N6 | 104CG | 4.516 |
| 300N6 | 168CD2 | 4.605 |
| 300N6 | 93OD2 | 4.758 |
| 300N6 | 233OE2 | 4.801 |
| 300N6 | 175ND1 | 4.884 |
| 300N6 | 76CE1 | 4.910 |
| 300N6 | 202OE2 | 4.964 |
| 168NE2 | 300N6 | 60.66 |
| 168NE2 | 300N6 | 50.92 |
| 168NE2 | 300N6 | 56.93 |
| 168NE2 | 300N6 | 94.08 |
| 168NE2 | 300N6 | 15.28 |
| 168NE2 | 300N6 | 74.77 |
| 168NE2 | 300N6 | 72.46 |
| 168NE2 | 300N6 | 79.88 |
| 168NE2 | 300N6 | 48.07 |
| 168NE2 | 300N6 | 65.13 |
| 168NE2 | 300N6 | 14.11 |
| 168NE2 | 300N6 | 112.91 |
| 168NE2 | 300N6 | 56.29 |
| 168NE2 | 300N6 | 57.85 |
| 168NE2 | 300N6 | 130.73 |
| 168NE2 | 300N6 | 70.53 |
| 175CE1 | 300N6 | 98.09 |
| 175CE1 | 300N6 | 17.84 |
| 175CE1 | 300N6 | 54.88 |
| 175CE1 | 300N6 | 58.33 |
| 175CE1 | 300N6 | 48.25 |
| 175CE1 | 300N6 | 64.93 |
| 175CE1 | 300N6 | 122.48 |
| 175CE1 | 300N6 | 98.82 |
| 175CE1 | 300N6 | 107.72 |
| 175CE1 | 300N6 | 56.36 |
| 175CE1 | 300N6 | 173.40 |
| 175CE1 | 300N6 | 116.81 |
| 175CE1 | 300N6 | 9.41 |
| 175CE1 | 300N6 | 103.10 |
| 175CE1 | 300N6 | 125.01 |
| 104OD2 | 300N6 | 103.34 |
| 104OD2 | 300N6 | 89.62 |
| 104OD2 | 300N6 | 43.45 |
| 104OD2 | 300N6 | 73.82 |
| 104OD2 | 300N6 | 56.73 |
| 104OD2 | 300N6 | 29.29 |
| 104OD2 | 300N6 | 65.42 |
| 104OD2 | 300N6 | 14.26 |
| 104OD2 | 300N6 | 64.32 |
| 104OD2 | 300N6 | 77.65 |
| 104OD2 | 300N6 | 35.43 |
| 104OD2 | 300N6 | 90.27 |
| 104OD2 | 300N6 | 154.38 |
| 104OD2 | 300N6 | 66.17 |
| 175NE2 | 300N6 | 72.37 |
| 175NE2 | 300N6 | 60.10 |
| 175NE2 | 300N6 | 65.80 |
| 175NE2 | 300N6 | 81.37 |
| 175NE2 | 300N6 | 131.56 |
| 175NE2 | 300N6 | 85.55 |
| 175NE2 | 300N6 | 115.66 |
| 175NE2 | 300N6 | 48.21 |
| 175NE2 | 300N6 | 158.07 |
| 175NE2 | 300N6 | 111.55 |
| 175NE2 | 300N6 | 25.94 |
| 175NE2 | 300N6 | 93.02 |
| 175NE2 | 300N6 | 111.10 |
| 174CD2 | 300N6 | 81.04 |
| 174CD2 | 300N6 | 19.90 |
| 174CD2 | 300N6 | 32.93 |
| 174CD2 | 300N6 | 91.82 |
| 174CD2 | 300N6 | 142.03 |
| 174CD2 | 300N6 | 86.97 |
| 174CD2 | 300N6 | 99.59 |
| 174CD2 | 300N6 | 129.48 |
| 174CD2 | 300N6 | 125.05 |
| 174CD2 | 300N6 | 49.15 |
| 174CD2 | 300N6 | 114.34 |
| 174CD2 | 300N6 | 155.77 |
| 168CE1 | 300N6 | 61.25 |
| 168CE1 | 300N6 | 57.27 |
| 168CE1 | 300N6 | 72.53 |
| 168CE1 | 300N6 | 61.10 |
| 168CE1 | 300N6 | 56.75 |
| 168CE1 | 300N6 | 28.01 |
| 168CE1 | 300N6 | 115.95 |
| 168CE1 | 300N6 | 59.87 |
| 168CE1 | 300N6 | 52.93 |
| 168CE1 | 300N6 | 144.64 |
| 168CE1 | 300N6 | 80.57 |
| 174CG | 300N6 | 19.60 |
| 174CG | 300N6 | 83.78 |
| 174CG | 300N6 | 122.35 |
| 174CG | 300N6 | 74.67 |
| 174CG | 300N6 | 81.82 |
| 174CG | 300N6 | 133.59 |
| 174CG | 300N6 | 108.52 |
| 174CG | 300N6 | 39.92 |
| 174CG | 300N6 | 131.50 |
| 174CG | 300N6 | 138.00 |
| 174CD1 | 300N6 | 64.19 |
| 174CD1 | 300N6 | 114.98 |
| 174CD1 | 300N6 | 55.61 |
| 174CD1 | 300N6 | 83.26 |
| 174CD1 | 300N6 | 115.65 |
| 174CD1 | 300N6 | 92.15 |
| 174CD1 | 300N6 | 55.79 |
| 174CD1 | 300N6 | 146.89 |
| 174CD1 | 300N6 | 122.84 |
| 104OD1 | 300N6 | 80.00 |
| 104OD1 | 300N6 | 15.94 |
| 104OD1 | 300N6 | 92.72 |
| 104OD1 | 300N6 | 55.13 |
| 104OD1 | 300N6 | 42.79 |
| 104OD1 | 300N6 | 113.43 |
| 104OD1 | 300N6 | 134.33 |
| 104OD1 | 300N6 | 67.63 |
| 202OE1 | 300N6 | 75.06 |
| 202OE1 | 300N6 | 44.66 |
| 202OE1 | 300N6 | 74.87 |
| 202OE1 | 300N6 | 37.23 |
| 202OE1 | 300N6 | 100.39 |
| 202OE1 | 300N6 | 96.91 |
| 202OE1 | 300N6 | 26.19 |
| 104CG | 300N6 | 78.58 |
| 104CG | 300N6 | 69.09 |
| 104CG | 300N6 | 40.23 |
| 104CG | 300N6 | 99.04 |
| 104CG | 300N6 | 148.97 |
| 104CG | 300N6 | 69.75 |
| 168CD2 | 300N6 | 117.04 |
| 168CD2 | 300N6 | 63.34 |
| 168CD2 | 300N6 | 56.18 |
| 168CD2 | 300N6 | 116.95 |
| 168CD2 | 300N6 | 70.04 |
| 93OD2 | 300N6 | 56.91 |
| 93OD2 | 300N6 | 167.91 |
| 93OD2 | 300N6 | 79.88 |
| 93OD2 | 300N6 | 48.71 |
| 233OE2 | 300N6 | 112.67 |
| 233OE2 | 300N6 | 119.92 |
| 233OE2 | 300N6 | 30.74 |
| 175ND1 | 300N6 | 111.94 |
| 175ND1 | 300N6 | 126.20 |
| 76CE1 | 300N6 | 89.70 |
| 300C7 | 76NE2 | 3.531 |
| 300C7 | 76CD2 | 3.776 |
| 300C7 | 76CE1 | 3.893 |
| 300C7 | 60CG | 4.209 |
| 300C7 | 76CG | 4.261 |
| 300C7 | 76ND1 | 4.304 |
| 300C7 | 67SG | 4.644 |
| 300C7 | 60CD | 4.881 |
| 76NE2 | 300C7 | 21.35 |
| 76NE2 | 300C7 | 19.73 |
| 76NE2 | 300C7 | 138.78 |
| 76NE2 | 300C7 | 30.99 |
| 76NE2 | 300C7 | 29.72 |
| 76NE2 | 300C7 | 91.58 |
| 76NE2 | 300C7 | 124.87 |
| 76CD2 | 300C7 | 33.21 |
| 76CD2 | 300C7 | 133.48 |
| 76CD2 | 300C7 | 18.17 |
| 76CD2 | 300C7 | 30.49 |
| 76CD2 | 300C7 | 82.30 |
| 76CD2 | 300C7 | 126.15 |
| 76CE1 | 300C7 | 157.19 |
| 76CE1 | 300C7 | 30.96 |
| 76CE1 | 300C7 | 17.67 |
| 76CE1 | 300C7 | 77.73 |
| 76CE1 | 300C7 | 141.05 |
| 60CG | 300C7 | 146.04 |
| 60CG | 300C7 | 163.78 |
| 60CG | 300C7 | 123.74 |
| 60CG | 300C7 | 17.36 |
| 76CG | 300C7 | 18.51 |
| 76CG | 300C7 | 64.49 |
| 76CG | 300C7 | 143.06 |
| 76ND1 | 300C7 | 62.25 |
| 76ND1 | 300C7 | 154.30 |
| 67SG | 300C7 | 140.97 |
| 300C8 | 76NE2 | 3.382 |
| 300C8 | 76CE1 | 3.902 |
| 300C8 | 76CD2 | 4.016 |
| 300C8 | 60CG | 4.248 |
| 300C8 | 175CE1 | 4.419 |
| 300C8 | 60CD | 4.465 |
| 300C8 | 60OE1 | 4.598 |
| 300C8 | 174CD2 | 4.684 |
| 300C8 | 76ND1 | 4.701 |
| 300C8 | 175NE2 | 4.751 |
| 300C8 | 76CG | 4.789 |
| 76NE2 | 300C8 | 19.24 |
| 76NE2 | 300C8 | 19.05 |
| 76NE2 | 300C8 | 143.37 |
| 76NE2 | 300C8 | 117.65 |
| 76NE2 | 300C8 | 144.61 |
| 76NE2 | 300C8 | 129.57 |
| 76NE2 | 300C8 | 148.21 |
| 76NE2 | 300C8 | 24.47 |
| 76NE2 | 300C8 | 103.88 |
| 76NE2 | 300C8 | 24.20 |
| 76CE1 | 300C8 | 32.14 |
| 76CE1 | 300C8 | 154.08 |
| 76CE1 | 300C8 | 110.22 |
| 76CE1 | 300C8 | 163.30 |
| 76CE1 | 300C8 | 148.78 |
| 76CE1 | 300C8 | 129.48 |
| 76CE1 | 300C8 | 14.15 |
| 76CE1 | 300C8 | 99.96 |
| 76CE1 | 300C8 | 27.00 |
| 76CD2 | 300C8 | 125.22 |
| 76CD2 | 300C8 | 136.55 |
| 76CD2 | 300C8 | 131.50 |
| 76CD2 | 300C8 | 119.07 |
| 76CD2 | 300C8 | 156.45 |
| 76CD2 | 300C8 | 27.63 |
| 76CD2 | 300C8 | 122.15 |
| 76CD2 | 300C8 | 14.59 |
| 60CG | 300C8 | 95.50 |
| 60CG | 300C8 | 19.94 |
| 60CG | 300C8 | 31.19 |
| 60CG | 300C8 | 66.15 |
| 60CG | 300C8 | 140.67 |
| 60CG | 300C8 | 105.86 |
| 60CG | 300C8 | 127.27 |
| 175CE1 | 300C8 | 82.18 |
| 175CE1 | 300C8 | 87.00 |
| 175CE1 | 300C8 | 48.76 |
| 175CE1 | 300C8 | 122.22 |
| 175CE1 | 300C8 | 16.06 |
| 175CE1 | 300C8 | 137.20 |
| 60CD | 300C8 | 15.83 |
| 60CD | 300C8 | 67.06 |
| 60CD | 300C8 | 155.42 |
| 60CD | 300C8 | 89.46 |
| 60CD | 300C8 | 139.14 |
| 60OE1 | 300C8 | 81.55 |
| 60OE1 | 300C8 | 146.09 |
| 60OE1 | 300C8 | 90.17 |
| 60OE1 | 300C8 | 129.64 |
| 174CD2 | 300C8 | 129.77 |
| 174CD2 | 300C8 | 64.81 |
| 174CD2 | 300C8 | 141.98 |
| 76ND1 | 300C8 | 113.41 |
| 76ND1 | 300C8 | 16.66 |
| 175NE2 | 300C8 | 125.92 |
| 300N9 | 104OD1 | 2.823 |
| 300N9 | 93OD1 | 2.941 |
| 300N9 | 93OD2 | 3.170 |
| 300N9 | 104OD2 | 3.207 |
| 300N9 | 93CG | 3.326 |
| 300N9 | 104CG | 3.354 |
| 300N9 | 233OE1 | 3.535 |
| 300N9 | 233OE2 | 4.047 |
| 300N9 | 233CD | 4.191 |
| 300N9 | 95OG | 4.412 |
| 300N9 | 95CB | 4.537 |
| 300N9 | 202OE2 | 4.605 |
| 300N9 | 93CB | 4.650 |
| 300N9 | 174CD1 | 4.735 |
| 300N9 | 168NE2 | 4.747 |
| 300N9 | 104CB | 4.792 |
| 300N9 | 202OE1 | 4.987 |
| 104OD1 | 300N9 | 67.33 |
| 104OD1 | 300N9 | 91.27 |
| 104OD1 | 300N9 | 42.33 |
| 104OD1 | 300N9 | 84.17 |
| 104OD1 | 300N9 | 21.21 |
| 104OD1 | 300N9 | 54.45 |
| 104OD1 | 300N9 | 56.07 |
| 104OD1 | 300N9 | 50.71 |
| 104OD1 | 300N9 | 73.08 |
| 104OD1 | 300N9 | 55.01 |
| 104OD1 | 300N9 | 87.17 |
| 104OD1 | 300N9 | 92.10 |
| 104OD1 | 300N9 | 73.50 |
| 104OD1 | 300N9 | 84.53 |
| 104OD1 | 300N9 | 21.22 |
| 104OD1 | 300N9 | 91.43 |
| 93OD1 | 300N9 | 41.87 |
| 93OD1 | 300N9 | 108.30 |
| 93OD1 | 300N9 | 21.87 |
| 93OD1 | 300N9 | 88.49 |
| 93OD1 | 300N9 | 60.03 |
| 93OD1 | 300N9 | 91.88 |
| 93OD1 | 300N9 | 74.45 |
| 93OD1 | 300N9 | 79.50 |
| 93OD1 | 300N9 | 74.48 |
| 93OD1 | 300N9 | 92.50 |
| 93OD1 | 300N9 | 25.77 |
| 93OD1 | 300N9 | 123.11 |
| 93OD1 | 300N9 | 145.77 |
| 93OD1 | 300N9 | 87.91 |
| 93OD1 | 300N9 | 117.70 |
| 93OD2 | 300N9 | 116.25 |
| 93OD2 | 300N9 | 21.96 |
| 93OD2 | 300N9 | 107.51 |
| 93OD2 | 300N9 | 48.11 |
| 93OD2 | 300N9 | 77.20 |
| 93OD2 | 300N9 | 63.02 |
| 93OD2 | 300N9 | 119.67 |
| 93OD2 | 300N9 | 116.34 |
| 93OD2 | 300N9 | 58.74 |
| 93OD2 | 300N9 | 27.95 |
| 93OD2 | 300N9 | 163.32 |
| 93OD2 | 300N9 | 124.75 |
| 93OD2 | 300N9 | 111.10 |
| 93OD2 | 300N9 | 84.27 |
| 104OD2 | 300N9 | 120.36 |
| 104OD2 | 300N9 | 21.90 |
| 104OD2 | 300N9 | 68.14 |
| 104OD2 | 300N9 | 43.26 |
| 104OD2 | 300N9 | 53.81 |
| 104OD2 | 300N9 | 90.32 |
| 104OD2 | 300N9 | 75.87 |
| 104OD2 | 300N9 | 74.95 |
| 104OD2 | 300N9 | 130.34 |
| 104OD2 | 300N9 | 56.27 |
| 104OD2 | 300N9 | 42.37 |
| 104OD2 | 300N9 | 25.94 |
| 104OD2 | 300N9 | 62.73 |
| 93CG | 300N9 | 104.29 |
| 93CG | 300N9 | 57.69 |
| 93CG | 300N9 | 90.42 |
| 93CG | 300N9 | 73.65 |
| 93CG | 300N9 | 97.71 |
| 93CG | 300N9 | 95.53 |
| 93CG | 300N9 | 79.21 |
| 93CG | 300N9 | 10.65 |
| 93CG | 300N9 | 144.65 |
| 93CG | 300N9 | 143.85 |
| 93CG | 300N9 | 105.39 |
| 93CG | 300N9 | 105.18 |
| 104CG | 300N9 | 63.05 |
| 104CG | 300N9 | 50.44 |
| 104CG | 300N9 | 53.11 |
| 104CG | 300N9 | 77.28 |
| 104CG | 300N9 | 60.27 |
| 104CG | 300N9 | 84.53 |
| 104CG | 300N9 | 112.87 |
| 104CG | 300N9 | 59.66 |
| 104CG | 300N9 | 64.22 |
| 104CG | 300N9 | 6.81 |
| 104CG | 300N9 | 79.83 |
| 233OE1 | 300N9 | 33.02 |
| 233OE1 | 300N9 | 15.97 |
| 233OE1 | 300N9 | 122.00 |
| 233OE1 | 300N9 | 105.69 |
| 233OE1 | 300N9 | 40.30 |
| 233OE1 | 300N9 | 68.23 |
| 233OE1 | 300N9 | 122.29 |
| 233OE1 | 300N9 | 88.11 |
| 233OE1 | 300N9 | 68.47 |
| 233OE1 | 300N9 | 60.24 |
| 233OE2 | 300N9 | 17.51 |
| 233OE2 | 300N9 | 127.34 |
| 233OE2 | 300N9 | 109.60 |
| 233OE2 | 300N9 | 34.11 |
| 233OE2 | 300N9 | 100.77 |
| 233OE2 | 300N9 | 98.97 |
| 233OE2 | 300N9 | 55.17 |
| 233OE2 | 300N9 | 57.25 |
| 233OE2 | 300N9 | 36.75 |
| 233CD | 300N9 | 123.46 |
| 233CD | 300N9 | 105.55 |
| 233CD | 300N9 | 36.85 |
| 233CD | 300N9 | 84.19 |
| 233CD | 300N9 | 109.67 |
| 233CD | 300N9 | 72.63 |
| 233CD | 300N9 | 59.40 |
| 233CD | 300N9 | 49.92 |
| 95OG | 300N9 | 18.09 |
| 95OG | 300N9 | 160.24 |
| 95OG | 300N9 | 93.26 |
| 95OG | 300N9 | 50.01 |
| 95OG | 300N9 | 111.54 |
| 95OG | 300N9 | 70.50 |
| 95OG | 300N9 | 150.92 |
| 95CB | 300N9 | 142.18 |
| 95CB | 300N9 | 94.31 |
| 95CB | 300N9 | 49.20 |
| 95CB | 300N9 | 105.67 |
| 95CB | 300N9 | 53.61 |
| 95CB | 300N9 | 138.60 |
| 202OE2 | 300N9 | 86.68 |
| 202OE2 | 300N9 | 125.40 |
| 202OE2 | 300N9 | 66.02 |
| 202OE2 | 300N9 | 91.34 |
| 202OE2 | 300N9 | 26.02 |
| 93CB | 300N9 | 142.82 |
| 93CB | 300N9 | 152.59 |
| 93CB | 300N9 | 113.12 |
| 93CB | 300N9 | 112.17 |
| 174CD1 | 300N9 | 61.74 |
| 174CD1 | 300N9 | 55.02 |
| 174CD1 | 300N9 | 102.49 |
| 168NE2 | 300N9 | 67.39 |
| 168NE2 | 300N9 | 41.02 |
| 104CB | 300N9 | 86.09 |
| 300N10 | 104OD2 | 2.710 |
| 300N10 | 104OD1 | 3.278 |
| 300N10 | 104CG | 3.343 |
| 300N10 | 168NE2 | 3.490 |
| 300N10 | 233OE2 | 3.603 |
| 300N10 | 233OE1 | 3.825 |
| 300N10 | 93OD2 | 3.990 |
| 300N10 | 202OE1 | 4.012 |
| 300N10 | 233CD | 4.132 |
| 300N10 | 93OD1 | 4.150 |
| 300N10 | 168CE1 | 4.163 |
| 300N10 | 202OE2 | 4.169 |
| 300N10 | 93CG | 4.430 |
| 300N10 | 174CD1 | 4.463 |
| 300N10 | 202CD | 4.476 |
| 300N10 | 168CD2 | 4.612 |
| 300N10 | 175CE1 | 4.700 |
| 300N10 | 104CB | 4.781 |
| 300N10 | 174CG | 4.868 |
| 300N10 | 174CD2 | 4.974 |
| 104OD2 | 300N10 | 41.92 |
| 104OD2 | 300N10 | 20.72 |
| 104OD2 | 300N10 | 60.90 |
| 104OD2 | 300N10 | 49.96 |
| 104OD2 | 300N10 | 68.40 |
| 104OD2 | 300N10 | 106.27 |
| 104OD2 | 300N10 | 82.23 |
| 104OD2 | 300N10 | 56.26 |
| 104OD2 | 300N10 | 90.74 |
| 104OD2 | 300N10 | 48.25 |
| 104OD2 | 300N10 | 87.71 |
| 104OD2 | 300N10 | 102.43 |
| 104OD2 | 300N10 | 61.83 |
| 104OD2 | 300N10 | 81.05 |
| 104OD2 | 300N10 | 70.97 |
| 104OD2 | 300N10 | 97.18 |
| 104OD2 | 300N10 | 18.37 |
| 104OD2 | 300N10 | 76.46 |
| 104OD2 | 300N10 | 91.88 |
| 104OD1 | 300N10 | 21.75 |
| 104OD1 | 300N10 | 102.69 |
| 104OD1 | 300N10 | 59.10 |
| 104OD1 | 300N10 | 48.81 |
| 104OD1 | 300N10 | 71.62 |
| 104OD1 | 300N10 | 104.77 |
| 104OD1 | 300N10 | 50.39 |
| 104OD1 | 300N10 | 49.29 |
| 104OD1 | 300N10 | 89.80 |
| 104OD1 | 300N10 | 89.52 |
| 104OD1 | 300N10 | 62.88 |
| 104OD1 | 300N10 | 74.41 |
| 104OD1 | 300N10 | 94.20 |
| 104OD1 | 300N10 | 112.21 |
| 104OD1 | 300N10 | 130.06 |
| 104OD1 | 300N10 | 27.15 |
| 104OD1 | 300N10 | 92.46 |
| 104OD1 | 300N10 | 98.46 |
| 104CG | 300N10 | 81.55 |
| 104CG | 300N10 | 54.99 |
| 104CG | 300N10 | 59.95 |
| 104CG | 300N10 | 91.28 |
| 104CG | 300N10 | 96.30 |
| 104CG | 300N10 | 53.81 |
| 104CG | 300N10 | 71.03 |
| 104CG | 300N10 | 68.08 |
| 104CG | 300N10 | 91.96 |
| 104CG | 300N10 | 84.23 |
| 104CG | 300N10 | 63.17 |
| 104CG | 300N10 | 90.60 |
| 104CG | 300N10 | 91.69 |
| 104CG | 300N10 | 111.76 |
| 104CG | 300N10 | 6.87 |
| 104CG | 300N10 | 80.73 |
| 104CG | 300N10 | 92.02 |
| 168NE2 | 300N10 | 70.99 |
| 168NE2 | 300N10 | 105.44 |
| 168NE2 | 300N10 | 140.94 |
| 168NE2 | 300N10 | 53.66 |
| 168NE2 | 300N10 | 87.94 |
| 168NE2 | 300N10 | 149.13 |
| 168NE2 | 300N10 | 17.13 |
| 168NE2 | 300N10 | 82.91 |
| 168NE2 | 300N10 | 151.96 |
| 168NE2 | 300N10 | 74.30 |
| 168NE2 | 300N10 | 66.81 |
| 168NE2 | 300N10 | 11.35 |
| 168NE2 | 300N10 | 52.09 |
| 168NE2 | 300N10 | 77.85 |
| 168NE2 | 300N10 | 70.92 |
| 168NE2 | 300N10 | 86.03 |
| 233OE2 | 300N10 | 34.46 |
| 233OE2 | 300N10 | 73.52 |
| 233OE2 | 300N10 | 45.76 |
| 233OE2 | 300N10 | 17.07 |
| 233OE2 | 300N10 | 81.56 |
| 233OE2 | 300N10 | 72.64 |
| 233OE2 | 300N10 | 38.11 |
| 233OE2 | 300N10 | 81.06 |
| 233OE2 | 300N10 | 111.67 |
| 233OE2 | 300N10 | 36.07 |
| 233OE2 | 300N10 | 72.78 |
| 233OE2 | 300N10 | 123.05 |
| 233OE2 | 300N10 | 59.52 |
| 233OE2 | 300N10 | 124.48 |
| 233OE2 | 300N10 | 141.35 |
| 233OE1 | 300N10 | 41.19 |
| 233OE1 | 300N10 | 69.28 |
| 233OE1 | 300N10 | 17.60 |
| 233OE1 | 300N10 | 48.36 |
| 233OE1 | 300N10 | 105.44 |
| 233OE1 | 300N10 | 43.49 |
| 233OE1 | 300N10 | 46.65 |
| 233OE1 | 300N10 | 122.31 |
| 233OE1 | 300N10 | 54.15 |
| 233OE1 | 300N10 | 106.61 |
| 233OE1 | 300N10 | 157.49 |
| 233OE1 | 300N10 | 66.72 |
| 233OE1 | 300N10 | 140.34 |
| 233OE1 | 300N10 | 146.63 |
| 93OD2 | 300N10 | 89.54 |
| 93OD2 | 300N10 | 58.07 |
| 93OD2 | 300N10 | 31.19 |
| 93OD2 | 300N10 | 146.13 |
| 93OD2 | 300N10 | 58.88 |
| 93OD2 | 300N10 | 15.95 |
| 93OD2 | 300N10 | 135.45 |
| 93OD2 | 300N10 | 74.99 |
| 93OD2 | 300N10 | 136.21 |
| 93OD2 | 300N10 | 156.51 |
| 93OD2 | 300N10 | 97.79 |
| 93OD2 | 300N10 | 145.88 |
| 93OD2 | 300N10 | 132.77 |
| 202OE1 | 300N10 | 57.83 |
| 202OE1 | 300N10 | 114.55 |
| 202OE1 | 300N10 | 67.58 |
| 202OE1 | 300N10 | 31.00 |
| 202OE1 | 300N10 | 104.51 |
| 202OE1 | 300N10 | 126.87 |
| 202OE1 | 300N10 | 15.72 |
| 202OE1 | 300N10 | 46.72 |
| 202OE1 | 300N10 | 92.31 |
| 202OE1 | 300N10 | 98.44 |
| 202OE1 | 300N10 | 124.15 |
| 202OE1 | 300N10 | 136.70 |
| 233CD | 300N10 | 64.63 |
| 233CD | 300N10 | 88.20 |
| 233CD | 300N10 | 39.53 |
| 233CD | 300N10 | 64.23 |
| 233CD | 300N10 | 116.31 |
| 233CD | 300N10 | 44.64 |
| 233CD | 300N10 | 89.80 |
| 233CD | 300N10 | 140.03 |
| 233CD | 300N10 | 59.85 |
| 233CD | 300N10 | 132.54 |
| 233CD | 300N10 | 145.82 |
| 93OD1 | 300N10 | 138.97 |
| 93OD1 | 300N10 | 84.08 |
| 93OD1 | 300N10 | 16.30 |
| 93OD1 | 300N10 | 104.29 |
| 93OD1 | 300N10 | 98.85 |
| 93OD1 | 300N10 | 154.22 |
| 93OD1 | 300N10 | 152.83 |
| 93OD1 | 300N10 | 76.16 |
| 93OD1 | 300N10 | 116.69 |
| 93OD1 | 300N10 | 108.35 |
| 168CE1 | 300N10 | 93.99 |
| 168CE1 | 300N10 | 149.41 |
| 168CE1 | 300N10 | 59.33 |
| 168CE1 | 300N10 | 78.57 |
| 168CE1 | 300N10 | 28.38 |
| 168CE1 | 300N10 | 53.85 |
| 168CE1 | 300N10 | 63.39 |
| 168CE1 | 300N10 | 59.66 |
| 168CE1 | 300N10 | 76.62 |
| 202OE2 | 300N10 | 73.54 |
| 202OE2 | 300N10 | 148.09 |
| 202OE2 | 300N10 | 16.15 |
| 202OE2 | 300N10 | 77.36 |
| 202OE2 | 300N10 | 122.04 |
| 202OE2 | 300N10 | 97.16 |
| 202OE2 | 300N10 | 153.55 |
| 202OE2 | 300N10 | 167.57 |
| 93CG | 300N10 | 119.77 |
| 93CG | 300N10 | 89.39 |
| 93CG | 300N10 | 150.42 |
| 93CG | 300N10 | 155.61 |
| 93CG | 300N10 | 90.02 |
| 93CG | 300N10 | 130.17 |
| 93CG | 300N10 | 118.60 |
| 174CD1 | 300N10 | 135.87 |
| 174CD1 | 300N10 | 83.41 |
| 174CD1 | 300N10 | 58.32 |
| 174CD1 | 300N10 | 56.74 |
| 174CD1 | 300N10 | 18.11 |
| 174CD1 | 300N10 | 30.15 |
| 202CD | 300N10 | 61.30 |
| 202CD | 300N10 | 108.03 |
| 202CD | 300N10 | 94.35 |
| 202CD | 300N10 | 137.65 |
| 202CD | 300N10 | 152.01 |
| 168CD2 | 300N10 | 51.16 |
| 168CD2 | 300N10 | 88.46 |
| 168CD2 | 300N10 | 77.49 |
| 168CD2 | 300N10 | 90.76 |
| 175CE1 | 300N10 | 105.07 |
| 175CE1 | 300N10 | 41.85 |
| 175CE1 | 300N10 | 45.70 |
| 104CB | 300N10 | 74.10 |
| 104CB | 300N10 | 86.01 |
| 174CG | 300N10 | 17.78 |
| 300C11 | 76CD2 | 3.744 |
| 300C11 | 60CG | 3.891 |
| 300C11 | 76NE2 | 3.979 |
| 300C11 | 62CD1 | 4.036 |
| 300C11 | 219CZ3 | 4.251 |
| 300C11 | 60CB | 4.278 |
| 300C11 | 76CG | 4.284 |
| 300C11 | 219CE3 | 4.363 |
| 300C11 | 62CE1 | 4.568 |
| 300C11 | 76CE1 | 4.592 |
| 300C11 | 62CG | 4.643 |
| 300C11 | 60CD | 4.741 |
| 300C11 | 76ND1 | 4.756 |
| 300C11 | 67SG | 4.789 |
| 300C11 | 62CB | 4.789 |
| 300C11 | 60OE1 | 4.843 |
| 76CD2 | 300C11 | 147.99 |
| 76CD2 | 300C11 | 20.21 |
| 76CD2 | 300C11 | 102.54 |
| 76CD2 | 300C11 | 97.90 |
| 76CD2 | 300C11 | 150.48 |
| 76CD2 | 300C11 | 17.87 |
| 76CD2 | 300C11 | 83.02 |
| 76CD2 | 300C11 | 86.24 |
| 76CD2 | 300C11 | 28.25 |
| 76CD2 | 300C11 | 116.59 |
| 76CD2 | 300C11 | 131.10 |
| 76CD2 | 300C11 | 26.53 |
| 76CD2 | 300C11 | 80.67 |
| 76CD2 | 300C11 | 134.24 |
| 76CD2 | 300C11 | 119.29 |
| 60CG | 300C11 | 134.17 |
| 60CG | 300C11 | 103.38 |
| 60CG | 300C11 | 81.89 |
| 60CG | 300C11 | 20.85 |
| 60CG | 300C11 | 164.60 |
| 60CG | 300C11 | 100.37 |
| 60CG | 300C11 | 120.68 |
| 60CG | 300C11 | 138.75 |
| 60CG | 300C11 | 92.45 |
| 60CG | 300C11 | 16.94 |
| 60CG | 300C11 | 154.08 |
| 60CG | 300C11 | 128.00 |
| 60CG | 300C11 | 74.10 |
| 60CG | 300C11 | 29.42 |
| 76NE2 | 300C11 | 121.28 |
| 76NE2 | 300C11 | 114.08 |
| 76NE2 | 300C11 | 147.32 |
| 76NE2 | 300C11 | 30.56 |
| 76NE2 | 300C11 | 101.46 |
| 76NE2 | 300C11 | 104.29 |
| 76NE2 | 300C11 | 15.73 |
| 76NE2 | 300C11 | 133.38 |
| 76NE2 | 300C11 | 117.95 |
| 76NE2 | 300C11 | 26.55 |
| 76NE2 | 300C11 | 84.24 |
| 76NE2 | 300C11 | 151.73 |
| 76NE2 | 300C11 | 109.87 |
| 62CD1 | 300C11 | 55.30 |
| 62CD1 | 300C11 | 85.01 |
| 62CD1 | 300C11 | 91.20 |
| 62CD1 | 300C11 | 48.79 |
| 62CD1 | 300C11 | 17.31 |
| 62CD1 | 300C11 | 117.35 |
| 62CD1 | 300C11 | 16.67 |
| 62CD1 | 300C11 | 117.57 |
| 62CD1 | 300C11 | 101.26 |
| 62CD1 | 300C11 | 63.98 |
| 62CD1 | 300C11 | 31.83 |
| 62CD1 | 300C11 | 119.05 |
| 219CZ3 | 300C11 | 62.77 |
| 219CZ3 | 300C11 | 102.91 |
| 219CZ3 | 300C11 | 18.54 |
| 219CZ3 | 300C11 | 62.87 |
| 219CZ3 | 300C11 | 126.14 |
| 219CZ3 | 300C11 | 64.74 |
| 219CZ3 | 300C11 | 84.31 |
| 219CZ3 | 300C11 | 119.38 |
| 219CZ3 | 300C11 | 117.40 |
| 219CZ3 | 300C11 | 62.83 |
| 219CZ3 | 300C11 | 75.66 |
| 60CB | 300C11 | 164.58 |
| 60CB | 300C11 | 80.87 |
| 60CB | 300C11 | 102.11 |
| 60CB | 300C11 | 157.43 |
| 60CB | 300C11 | 76.95 |
| 60CB | 300C11 | 32.56 |
| 60CB | 300C11 | 173.48 |
| 60CB | 300C11 | 127.26 |
| 60CB | 300C11 | 59.19 |
| 60CB | 300C11 | 37.72 |
| 76CG | 300C11 | 85.39 |
| 76CG | 300C11 | 73.99 |
| 76CG | 300C11 | 28.49 |
| 76CG | 300C11 | 102.86 |
| 76CG | 300C11 | 147.85 |
| 76CG | 300C11 | 16.49 |
| 76CG | 300C11 | 63.03 |
| 76CG | 300C11 | 121.19 |
| 76CG | 300C11 | 137.05 |
| 219CE3 | 300C11 | 50.69 |
| 219CE3 | 300C11 | 110.87 |
| 219CE3 | 300C11 | 62.59 |
| 219CE3 | 300C11 | 102.43 |
| 219CE3 | 300C11 | 101.85 |
| 219CE3 | 300C11 | 104.02 |
| 219CE3 | 300C11 | 67.18 |
| 219CE3 | 300C11 | 92.58 |
| 62CE1 | 300C11 | 100.13 |
| 62CE1 | 300C11 | 30.57 |
| 62CE1 | 300C11 | 134.62 |
| 62CE1 | 300C11 | 84.08 |
| 62CE1 | 300C11 | 54.56 |
| 62CE1 | 300C11 | 48.06 |
| 62CE1 | 300C11 | 134.23 |
| 76CE1 | 300C11 | 125.40 |
| 76CE1 | 300C11 | 124.99 |
| 76CE1 | 300C11 | 16.15 |
| 76CE1 | 300C11 | 70.09 |
| 76CE1 | 300C11 | 142.65 |
| 76CE1 | 300C11 | 120.40 |
| 62CG | 300C11 | 108.44 |
| 62CG | 300C11 | 109.57 |
| 62CG | 300C11 | 61.11 |
| 62CG | 300C11 | 18.35 |
| 62CG | 300C11 | 114.12 |
| 60CD | 300C11 | 141.14 |
| 60CD | 300C11 | 140.83 |
| 60CD | 300C11 | 90.17 |
| 60CD | 300C11 | 15.00 |
| 76ND1 | 300C11 | 58.10 |
| 76ND1 | 300C11 | 127.31 |
| 76ND1 | 300C11 | 135.83 |
| 67SG | 300C11 | 74.33 |
| 67SG | 300C11 | 155.83 |
| 62CB | 300C11 | 96.80 |
| 300C12 | 60OE1 | 3.657 |
| 300C12 | 76NE2 | 3.721 |
| 300C12 | 60CD | 3.875 |
| 300C12 | 60CG | 4.032 |
| 300C12 | 76CD2 | 4.255 |
| 300C12 | 60OE2 | 4.537 |
| 300C12 | 76CE1 | 4.599 |
| 300C12 | 175CE1 | 4.634 |
| 300C12 | 175NE2 | 4.756 |
| 300C12 | 60CB | 4.997 |
| 60OE1 | 300C12 | 157.70 |
| 60OE1 | 300C12 | 18.90 |
| 60OE1 | 300C12 | 35.99 |
| 60OE1 | 300C12 | 139.74 |
| 60OE1 | 300C12 | 28.67 |
| 60OE1 | 300C12 | 165.35 |
| 60OE1 | 300C12 | 96.27 |
| 60OE1 | 300C12 | 103.04 |
| 60OE1 | 300C12 | 36.55 |
| 76NE2 | 300C12 | 160.15 |
| 76NE2 | 300C12 | 138.41 |
| 76NE2 | 300C12 | 18.32 |
| 76NE2 | 300C12 | 165.68 |
| 76NE2 | 300C12 | 13.70 |
| 76NE2 | 300C12 | 106.02 |
| 76NE2 | 300C12 | 98.52 |
| 76NE2 | 300C12 | 130.14 |
| 60CD | 300C12 | 22.11 |
| 60CD | 300C12 | 144.08 |
| 60CD | 300C12 | 14.58 |
| 60CD | 300C12 | 155.23 |
| 60CD | 300C12 | 86.18 |
| 60CD | 300C12 | 96.96 |
| 60CD | 300C12 | 30.42 |
| 60CG | 300C12 | 124.63 |
| 60CG | 300C12 | 31.63 |
| 60CG | 300C12 | 133.83 |
| 60CG | 300C12 | 95.29 |
| 60CG | 300C12 | 109.45 |
| 60CG | 300C12 | 15.14 |
| 76CD2 | 300C12 | 156.24 |
| 76CD2 | 300C12 | 28.36 |
| 76CD2 | 300C12 | 123.64 |
| 76CD2 | 300C12 | 116.84 |
| 76CD2 | 300C12 | 113.74 |
| 60OE2 | 300C12 | 153.54 |
| 60OE2 | 300C12 | 71.64 |
| 60OE2 | 300C12 | 82.51 |
| 60OE2 | 300C12 | 43.37 |
| 76CE1 | 300C12 | 95.45 |
| 76CE1 | 300C12 | 90.57 |
| 76CE1 | 300C12 | 130.01 |
| 175CE1 | 300C12 | 16.13 |
| 175CE1 | 300C12 | 110.37 |
| 175NE2 | 300C12 | 124.16 |
| 300C13 | 219CZ3 | 3.458 |
| 300C13 | 60CG | 3.606 |
| 300C13 | 219CE3 | 3.806 |
| 300C13 | 60CB | 3.831 |
| 300C13 | 60OE1 | 3.919 |
| 300C13 | 76CD2 | 3.963 |
| 300C13 | 60CD | 4.154 |
| 300C13 | 76NE2 | 4.248 |
| 300C13 | 62CD1 | 4.582 |
| 300C13 | 219CH2 | 4.648 |
| 300C13 | 76CG | 4.839 |
| 219CZ3 | 300C13 | 98.24 |
| 219CZ3 | 300C13 | 21.41 |
| 219CZ3 | 300C13 | 74.89 |
| 219CZ3 | 300C13 | 98.52 |
| 219CZ3 | 300C13 | 108.71 |
| 219CZ3 | 300C13 | 104.78 |
| 219CZ3 | 300C13 | 127.05 |
| 219CZ3 | 300C13 | 55.11 |
| 219CZ3 | 300C13 | 10.78 |
| 219CZ3 | 300C13 | 105.93 |
| 60CG | 300C13 | 117.77 |
| 60CG | 300C13 | 23.43 |
| 60CG | 300C13 | 36.93 |
| 60CG | 300C13 | 151.60 |
| 60CG | 300C13 | 21.17 |
| 60CG | 300C13 | 134.55 |
| 60CG | 300C13 | 98.17 |
| 60CG | 300C13 | 87.47 |
| 60CG | 300C13 | 146.80 |
| 219CE3 | 300C13 | 94.41 |
| 219CE3 | 300C13 | 119.19 |
| 219CE3 | 300C13 | 87.88 |
| 219CE3 | 300C13 | 126.17 |
| 219CE3 | 300C13 | 106.58 |
| 219CE3 | 300C13 | 47.95 |
| 219CE3 | 300C13 | 31.58 |
| 219CE3 | 300C13 | 84.51 |
| 60CB | 300C13 | 45.48 |
| 60CB | 300C13 | 168.93 |
| 60CB | 300C13 | 37.22 |
| 60CB | 300C13 | 157.45 |
| 60CB | 300C13 | 83.32 |
| 60CB | 300C13 | 64.11 |
| 60CB | 300C13 | 156.05 |
| 60OE1 | 300C13 | 141.03 |
| 60OE1 | 300C13 | 17.57 |
| 60OE1 | 300C13 | 124.77 |
| 60OE1 | 300C13 | 128.54 |
| 60OE1 | 300C13 | 89.96 |
| 60OE1 | 300C13 | 151.73 |
| 76CD2 | 300C13 | 144.65 |
| 76CD2 | 300C13 | 18.87 |
| 76CD2 | 300C13 | 90.26 |
| 76CD2 | 300C13 | 119.33 |
| 76CD2 | 300C13 | 13.57 |
| 60CD | 300C13 | 125.88 |
| 60CD | 300C13 | 118.60 |
| 60CD | 300C13 | 94.69 |
| 60CD | 300C13 | 149.20 |
| 76NE2 | 300C13 | 104.52 |
| 76NE2 | 300C13 | 137.79 |
| 76NE2 | 300C13 | 26.99 |
| 62CD1 | 300C13 | 56.52 |
| 62CD1 | 300C13 | 78.23 |
| 219CH2 | 300C13 | 115.76 |
| 300C14 | 60OE1 | 3.226 |
| 300C14 | 60CG | 3.682 |
| 300C14 | 60CD | 3.687 |
| 300C14 | 76NE2 | 4.132 |
| 300C14 | 76CD2 | 4.209 |
| 300C14 | 60CB | 4.238 |
| 300C14 | 219CZ3 | 4.431 |
| 300C14 | 60OE2 | 4.666 |
| 300C14 | 219CE3 | 4.879 |
| 60OE1 | 300C14 | 40.01 |
| 60OE1 | 300C14 | 19.48 |
| 60OE1 | 300C14 | 159.20 |
| 60OE1 | 300C14 | 176.13 |
| 60OE1 | 300C14 | 44.84 |
| 60OE1 | 300C14 | 92.59 |
| 60OE1 | 300C14 | 24.76 |
| 60OE1 | 300C14 | 108.89 |
| 60CG | 300C14 | 23.87 |
| 60CG | 300C14 | 136.08 |
| 60CG | 300C14 | 136.74 |
| 60CG | 300C14 | 20.73 |
| 60CG | 300C14 | 81.80 |
| 60CG | 300C14 | 30.39 |
| 60CG | 300C14 | 94.66 |
| 60CD | 300C14 | 146.21 |
| 60CD | 300C14 | 156.68 |
| 60CD | 300C14 | 36.97 |
| 60CD | 300C14 | 95.82 |
| 60CD | 300C14 | 10.83 |
| 60CD | 300C14 | 111.20 |
| 76NE2 | 300C14 | 18.94 |
| 76NE2 | 300C14 | 142.41 |
| 76NE2 | 300C14 | 107.48 |
| 76NE2 | 300C14 | 137.23 |
| 76NE2 | 300C14 | 91.26 |
| 76CD2 | 300C14 | 133.38 |
| 76CD2 | 300C14 | 88.62 |
| 76CD2 | 300C14 | 151.52 |
| 76CD2 | 300C14 | 72.36 |
| 60CB | 300C14 | 61.59 |
| 60CB | 300C14 | 46.67 |
| 60CB | 300C14 | 75.50 |
| 219CZ3 | 300C14 | 106.54 |
| 219CZ3 | 300C14 | 16.30 |
| 60OE2 | 300C14 | 121.70 |
| 253ZN | 104OD2 | 2.030 |
| 253ZN | 168NE2 | 2.220 |
| 253ZN | 233OE2 | 2.301 |
| 253ZN | 202OE1 | 2.663 |
| 253ZN | 168CE1 | 2.976 |
| 253ZN | 104CG | 3.105 |
| 253ZN | 202CD | 3.239 |
| 253ZN | 168CD2 | 3.295 |
| 253ZN | 233CD | 3.319 |
| 253ZN | 202OE2 | 3.413 |
| 253ZN | 104OD1 | 3.554 |
| 253ZN | 233OE1 | 3.643 |
| 253ZN | 168ND1 | 4.123 |
| 253ZN | 168CG | 4.306 |
| 253ZN | 104CB | 4.368 |
| 253ZN | 202CG | 4.439 |
| 253ZN | 233CG | 4.666 |
| 253ZN | 93OD2 | 4.924 |
| 104OD2 | 253ZN | 98.10 |
| 104OD2 | 253ZN | 79.80 |
| 104OD2 | 253ZN | 149.19 |
| 104OD2 | 253ZN | 74.01 |
| 104OD2 | 253ZN | 14.80 |
| 104OD2 | 253ZN | 133.12 |
| 104OD2 | 253ZN | 114.19 |
| 104OD2 | 253ZN | 76.43 |
| 104OD2 | 253ZN | 125.52 |
| 104OD2 | 253ZN | 34.58 |
| 104OD2 | 253ZN | 78.09 |
| 104OD2 | 253ZN | 82.40 |
| 104OD2 | 253ZN | 100.92 |
| 104OD2 | 253ZN | 7.30 |
| 104OD2 | 253ZN | 123.08 |
| 104OD2 | 253ZN | 73.89 |
| 104OD2 | 253ZN | 92.73 |
| 168NE2 | 253ZN | 131.31 |
| 168NE2 | 253ZN | 88.36 |
| 168NE2 | 253ZN | 24.30 |
| 168NE2 | 253ZN | 112.86 |
| 168NE2 | 253ZN | 108.18 |
| 168NE2 | 253ZN | 18.22 |
| 168NE2 | 253ZN | 146.31 |
| 168NE2 | 253ZN | 128.30 |
| 168NE2 | 253ZN | 131.18 |
| 168NE2 | 253ZN | 166.31 |
| 168NE2 | 253ZN | 18.64 |
| 168NE2 | 253ZN | 12.73 |
| 168NE2 | 253ZN | 101.89 |
| 168NE2 | 253ZN | 103.01 |
| 168NE2 | 253ZN | 135.74 |
| 168NE2 | 253ZN | 159.98 |
| 233OE2 | 253ZN | 73.52 |
| 233OE2 | 253ZN | 121.77 |
| 233OE2 | 253ZN | 71.23 |
| 233OE2 | 253ZN | 53.63 |
| 233OE2 | 253ZN | 123.02 |
| 233OE2 | 253ZN | 15.48 |
| 233OE2 | 253ZN | 49.42 |
| 233OE2 | 253ZN | 67.24 |
| 233OE2 | 253ZN | 35.31 |
| 233OE2 | 253ZN | 118.24 |
| 233OE2 | 253ZN | 119.25 |
| 233OE2 | 253ZN | 72.59 |
| 233OE2 | 253ZN | 47.04 |
| 233OE2 | 253ZN | 7.48 |
| 233OE2 | 253ZN | 67.16 |
| 202OE1 | 253ZN | 107.39 |
| 202OE1 | 253ZN | 144.62 |
| 202OE1 | 253ZN | 21.74 |
| 202OE1 | 253ZN | 70.17 |
| 202OE1 | 253ZN | 81.57 |
| 202OE1 | 253ZN | 39.94 |
| 202OE1 | 253ZN | 136.93 |
| 202OE1 | 253ZN | 88.54 |
| 202OE1 | 253ZN | 96.73 |
| 202OE1 | 253ZN | 79.57 |
| 202OE1 | 253ZN | 142.31 |
| 202OE1 | 253ZN | 27.09 |
| 202OE1 | 253ZN | 80.48 |
| 202OE1 | 253ZN | 90.92 |
| 168CE1 | 253ZN | 88.81 |
| 168CE1 | 253ZN | 123.47 |
| 168CE1 | 253ZN | 40.57 |
| 168CE1 | 253ZN | 132.89 |
| 168CE1 | 253ZN | 144.93 |
| 168CE1 | 253ZN | 107.88 |
| 168CE1 | 253ZN | 147.63 |
| 168CE1 | 253ZN | 10.75 |
| 168CE1 | 253ZN | 28.46 |
| 168CE1 | 253ZN | 77.60 |
| 168CE1 | 253ZN | 113.63 |
| 168CE1 | 253ZN | 122.92 |
| 168CE1 | 253ZN | 161.11 |
| 104CG | 253ZN | 124.49 |
| 104CG | 253ZN | 128.92 |
| 104CG | 253ZN | 64.96 |
| 104CG | 253ZN | 112.83 |
| 104CG | 253ZN | 20.25 |
| 104CG | 253ZN | 64.01 |
| 104CG | 253ZN | 97.12 |
| 104CG | 253ZN | 115.54 |
| 104CG | 253ZN | 13.04 |
| 104CG | 253ZN | 117.61 |
| 104CG | 253ZN | 64.48 |
| 104CG | 253ZN | 78.31 |
| 202CD | 253ZN | 90.35 |
| 202CD | 253ZN | 60.09 |
| 202CD | 253ZN | 21.49 |
| 202CD | 253ZN | 115.21 |
| 202CD | 253ZN | 67.19 |
| 202CD | 253ZN | 112.85 |
| 202CD | 253ZN | 97.86 |
| 202CD | 253ZN | 125.83 |
| 202CD | 253ZN | 14.34 |
| 202CD | 253ZN | 60.08 |
| 202CD | 253ZN | 75.41 |
| 168CD2 | 253ZN | 138.30 |
| 168CD2 | 253ZN | 110.10 |
| 168CD2 | 253ZN | 148.44 |
| 168CD2 | 253ZN | 155.56 |
| 168CD2 | 253ZN | 31.80 |
| 168CD2 | 253ZN | 13.72 |
| 168CD2 | 253ZN | 116.84 |
| 168CD2 | 253ZN | 86.59 |
| 168CD2 | 253ZN | 129.37 |
| 168CD2 | 253ZN | 152.00 |
| 233CD | 253ZN | 49.27 |
| 233CD | 253ZN | 56.27 |
| 233CD | 253ZN | 20.07 |
| 233CD | 253ZN | 131.49 |
| 233CD | 253ZN | 134.58 |
| 233CD | 253ZN | 69.79 |
| 233CD | 253ZN | 57.26 |
| 233CD | 253ZN | 10.68 |
| 233CD | 253ZN | 52.92 |
| 202OE2 | 253ZN | 98.59 |
| 202OE2 | 253ZN | 49.82 |
| 202OE2 | 253ZN | 134.33 |
| 202OE2 | 253ZN | 118.90 |
| 202OE2 | 253ZN | 119.05 |
| 202OE2 | 253ZN | 32.25 |
| 202OE2 | 253ZN | 53.59 |
| 202OE2 | 253ZN | 53.93 |
| 104OD1 | 253ZN | 48.85 |
| 104OD1 | 253ZN | 116.85 |
| 104OD1 | 253ZN | 135.46 |
| 104OD1 | 253ZN | 33.13 |
| 104OD1 | 253ZN | 113.44 |
| 104OD1 | 253ZN | 59.78 |
| 104OD1 | 253ZN | 58.16 |
| 233OE1 | 253ZN | 149.60 |
| 233OE1 | 253ZN | 154.55 |
| 233OE1 | 253ZN | 72.97 |
| 233OE1 | 253ZN | 69.26 |
| 233OE1 | 253ZN | 30.58 |
| 233OE1 | 253ZN | 33.44 |
| 168ND1 | 253ZN | 18.68 |
| 168ND1 | 253ZN | 85.16 |
| 168ND1 | 253ZN | 103.55 |
| 168ND1 | 253ZN | 120.91 |
| 168ND1 | 253ZN | 171.66 |
| 168CG | 253ZN | 103.24 |
| 168CG | 253ZN | 91.25 |
| 168CG | 253ZN | 124.30 |
| 168CG | 253ZN | 165.70 |
| 104CB | 253ZN | 115.89 |
| 104CB | 253ZN | 66.80 |
| 104CB | 253ZN | 90.83 |
| 202CG | 253ZN | 54.29 |
| 202CG | 253ZN | 84.78 |
| 233CG | 253ZN | 63.60 |
| 254ZN | 233OE1 | 1.901 |
| 254ZN | 93OD1 | 1.967 |
| 254ZN | 104OD1 | 2.171 |
| 254ZN | 93OD2 | 2.177 |
| 254ZN | 93CG | 2.351 |
| 254ZN | 233CD | 2.841 |
| 254ZN | 104CG | 3.093 |
| 254ZN | 233OE2 | 3.221 |
| 254ZN | 104OD2 | 3.343 |
| 254ZN | 93CB | 3.859 |
| 254ZN | 202OE2 | 3.953 |
| 254ZN | 233CG | 4.151 |
| 254ZN | 104CB | 4.485 |
| 254ZN | 93CA | 4.641 |
| 254ZN | 104C | 4.736 |
| 254ZN | 233CB | 4.765 |
| 254ZN | 202CD | 4.772 |
| 254ZN | 104CA | 4.854 |
| 254ZN | 93C | 4.873 |
| 254ZN | 95CB | 4.973 |
| 254ZN | 202OE1 | 4.997 |
| 233OE1 | 254ZN | 115.97 |
| 233OE1 | 254ZN | 93.69 |
| 233OE1 | 254ZN | 84.63 |
| 233OE1 | 254ZN | 101.91 |
| 233OE1 | 254ZN | 20.60 |
| 233OE1 | 254ZN | 89.10 |
| 233OE1 | 254ZN | 41.97 |
| 233OE1 | 254ZN | 87.96 |
| 233OE1 | 254ZN | 103.44 |
| 233OE1 | 254ZN | 46.39 |
| 233OE1 | 254ZN | 17.35 |
| 233OE1 | 254ZN | 87.62 |
| 233OE1 | 254ZN | 99.36 |
| 233OE1 | 254ZN | 70.88 |
| 233OE1 | 254ZN | 9.58 |
| 233OE1 | 254ZN | 49.53 |
| 233OE1 | 254ZN | 86.84 |
| 233OE1 | 254ZN | 112.39 |
| 233OE1 | 254ZN | 135.20 |
| 233OE1 | 254ZN | 62.68 |
| 93OD1 | 254ZN | 101.07 |
| 93OD1 | 254ZN | 63.70 |
| 93OD1 | 254ZN | 32.03 |
| 93OD1 | 254ZN | 133.29 |
| 93OD1 | 254ZN | 119.26 |
| 93OD1 | 254ZN | 155.93 |
| 93OD1 | 254ZN | 138.28 |
| 93OD1 | 254ZN | 30.19 |
| 93OD1 | 254ZN | 137.94 |
| 93OD1 | 254ZN | 120.64 |
| 93OD1 | 254ZN | 111.96 |
| 93OD1 | 254ZN | 19.84 |
| 93OD1 | 254ZN | 94.80 |
| 93OD1 | 254ZN | 107.63 |
| 93OD1 | 254ZN | 150.44 |
| 93OD1 | 254ZN | 95.80 |
| 93OD1 | 254ZN | 3.92 |
| 93OD1 | 254ZN | 70.60 |
| 93OD1 | 254ZN | 158.70 |
| 104OD1 | 254ZN | 161.45 |
| 104OD1 | 254ZN | 132.41 |
| 104OD1 | 254ZN | 79.55 |
| 104OD1 | 254ZN | 18.77 |
| 104OD1 | 254ZN | 75.53 |
| 104OD1 | 254ZN | 40.61 |
| 104OD1 | 254ZN | 130.77 |
| 104OD1 | 254ZN | 116.13 |
| 104OD1 | 254ZN | 76.43 |
| 104OD1 | 254ZN | 10.96 |
| 104OD1 | 254ZN | 114.56 |
| 104OD1 | 254ZN | 29.02 |
| 104OD1 | 254ZN | 90.96 |
| 104OD1 | 254ZN | 105.21 |
| 104OD1 | 254ZN | 11.53 |
| 104OD1 | 254ZN | 100.04 |
| 104OD1 | 254ZN | 43.85 |
| 104OD1 | 254ZN | 100.24 |
| 93OD2 | 254ZN | 31.67 |
| 93OD2 | 254ZN | 102.81 |
| 93OD2 | 254ZN | 173.72 |
| 93OD2 | 254ZN | 113.67 |
| 93OD2 | 254ZN | 157.10 |
| 93OD2 | 254ZN | 33.54 |
| 93OD2 | 254ZN | 75.68 |
| 93OD2 | 254ZN | 101.55 |
| 93OD2 | 254ZN | 167.83 |
| 93OD2 | 254ZN | 47.98 |
| 93OD2 | 254ZN | 136.30 |
| 93OD2 | 254ZN | 84.34 |
| 93OD2 | 254ZN | 87.76 |
| 93OD2 | 254ZN | 150.94 |
| 93OD2 | 254ZN | 64.01 |
| 93OD2 | 254ZN | 130.04 |
| 93OD2 | 254ZN | 95.40 |
| 93CG | 254ZN | 122.50 |
| 93CG | 254ZN | 151.03 |
| 93CG | 254ZN | 140.78 |
| 93CG | 254ZN | 168.95 |
| 93CG | 254ZN | 1.98 |
| 93CG | 254ZN | 106.83 |
| 93CG | 254ZN | 114.98 |
| 93CG | 254ZN | 142.99 |
| 93CG | 254ZN | 18.97 |
| 93CG | 254ZN | 118.73 |
| 93CG | 254ZN | 97.17 |
| 93CG | 254ZN | 119.15 |
| 93CG | 254ZN | 125.36 |
| 93CG | 254ZN | 32.54 |
| 93CG | 254ZN | 100.61 |
| 93CG | 254ZN | 126.93 |
| 233CD | 254ZN | 71.07 |
| 233CD | 254ZN | 22.94 |
| 233CD | 254ZN | 67.50 |
| 233CD | 254ZN | 124.03 |
| 233CD | 254ZN | 45.24 |
| 233CD | 254ZN | 13.34 |
| 233CD | 254ZN | 71.45 |
| 233CD | 254ZN | 118.86 |
| 233CD | 254ZN | 63.36 |
| 233CD | 254ZN | 26.67 |
| 233CD | 254ZN | 42.29 |
| 233CD | 254ZN | 75.58 |
| 233CD | 254ZN | 129.43 |
| 233CD | 254ZN | 123.38 |
| 233CD | 254ZN | 51.88 |
| 104CG | 254ZN | 61.18 |
| 104CG | 254ZN | 22.04 |
| 104CG | 254ZN | 149.31 |
| 104CG | 254ZN | 100.18 |
| 104CG | 254ZN | 72.17 |
| 104CG | 254ZN | 9.19 |
| 104CG | 254ZN | 133.32 |
| 104CG | 254ZN | 40.08 |
| 104CG | 254ZN | 89.42 |
| 104CG | 254ZN | 88.42 |
| 104CG | 254ZN | 27.56 |
| 104CG | 254ZN | 118.51 |
| 104CG | 254ZN | 55.21 |
| 104CG | 254ZN | 81.96 |
| 233OE2 | 254ZN | 50.18 |
| 233OE2 | 254ZN | 142.64 |
| 233OE2 | 254ZN | 40.80 |
| 233OE2 | 254ZN | 35.30 |
| 233OE2 | 254ZN | 65.17 |
| 233OE2 | 254ZN | 141.33 |
| 233OE2 | 254ZN | 70.48 |
| 233OE2 | 254ZN | 49.35 |
| 233OE2 | 254ZN | 31.54 |
| 233OE2 | 254ZN | 76.20 |
| 233OE2 | 254ZN | 152.01 |
| 233OE2 | 254ZN | 116.29 |
| 233OE2 | 254ZN | 34.78 |
| 104OD2 | 254ZN | 167.02 |
| 104OD2 | 254ZN | 83.53 |
| 104OD2 | 254ZN | 73.76 |
| 104OD2 | 254ZN | 31.07 |
| 104OD2 | 254ZN | 154.92 |
| 104OD2 | 254ZN | 59.63 |
| 104OD2 | 254ZN | 91.85 |
| 104OD2 | 254ZN | 71.08 |
| 104OD2 | 254ZN | 49.47 |
| 104OD2 | 254ZN | 138.44 |
| 104OD2 | 254ZN | 68.60 |
| 104OD2 | 254ZN | 62.07 |
| 93CB | 254ZN | 108.79 |
| 93CB | 254ZN | 116.21 |
| 93CB | 254ZN | 141.43 |
| 93CB | 254ZN | 17.95 |
| 93CB | 254ZN | 117.92 |
| 93CB | 254ZN | 98.50 |
| 93CB | 254ZN | 121.10 |
| 93CB | 254ZN | 123.97 |
| 93CB | 254ZN | 30.80 |
| 93CB | 254ZN | 98.63 |
| 93CB | 254ZN | 128.71 |
| 202OE2 | 254ZN | 56.03 |
| 202OE2 | 254ZN | 105.47 |
| 202OE2 | 254ZN | 118.78 |
| 202OE2 | 254ZN | 108.02 |
| 202OE2 | 254ZN | 55.62 |
| 202OE2 | 254ZN | 12.51 |
| 202OE2 | 254ZN | 116.78 |
| 202OE2 | 254ZN | 136.93 |
| 202OE2 | 254ZN | 151.23 |
| 202OE2 | 254ZN | 25.03 |
| 233CG | 254ZN | 70.30 |
| 233CG | 254ZN | 107.98 |
| 233CG | 254ZN | 55.26 |
| 233CG | 254ZN | 18.14 |
| 233CG | 254ZN | 54.84 |
| 233CG | 254ZN | 70.12 |
| 233CG | 254ZN | 116.73 |
| 233CG | 254ZN | 118.84 |
| 233CG | 254ZN | 65.14 |
| 104CB | 254ZN | 124.69 |
| 104CB | 254ZN | 31.57 |
| 104CB | 254ZN | 86.46 |
| 104CB | 254ZN | 94.35 |
| 104CB | 254ZN | 18.41 |
| 104CB | 254ZN | 110.83 |
| 104CB | 254ZN | 52.61 |
| 104CB | 254ZN | 89.34 |
| 93CA | 254ZN | 100.00 |
| 93CA | 254ZN | 92.20 |
| 93CA | 254ZN | 131.19 |
| 93CA | 254ZN | 106.65 |
| 93CA | 254ZN | 18.17 |
| 93CA | 254ZN | 89.97 |
| 93CA | 254ZN | 142.22 |
| 104C | 254ZN | 65.66 |
| 104C | 254ZN | 101.60 |
| 104C | 254ZN | 18.20 |
| 104C | 254ZN | 92.10 |
| 104C | 254ZN | 64.34 |
| 104C | 254ZN | 104.00 |
| 233CB | 254ZN | 59.11 |
| 233CB | 254ZN | 82.75 |
| 233CB | 254ZN | 103.93 |
| 233CB | 254ZN | 129.54 |
| 233CB | 254ZN | 72.20 |
| 202CD | 254ZN | 107.42 |
| 202CD | 254ZN | 149.36 |
| 202CD | 254ZN | 138.83 |
| 202CD | 254ZN | 14.45 |
| 104CA | 254ZN | 94.12 |
| 104CA | 254ZN | 48.74 |
| 104CA | 254ZN | 105.19 |
| 93C | 254ZN | 71.81 |
| 93C | 254ZN | 159.41 |
| 95CB | 254ZN | 126.71 |

TABLE VI
Provides bond angles between interresidue atoms that are within
5 Ångstroms apart in an active site of S. aureus methionine
aminopeptidase for an inhibitor complex with 5-(3-Iodo-
phenyl)-1-H-[1,2,3]triazole.

| Atom 1 | Atom 2 | Atom 3 | Angle |
|---|---|---|---|
| 174CD2 | 300C1 | 174CD1 | 38.87 |
| 174CD2 | 300C1 | 95OG | 93.98 |
| 174CD2 | 300C1 | 174CG | 19.13 |
| 174CD2 | 300C1 | 95CB | 90.77 |
| 174CD1 | 300C1 | 95OG | 62.23 |
| 174CD1 | 300C1 | 174CG | 20.05 |
| 174CD1 | 300C1 | 95CB | 54.57 |
| 95OG | 300C1 | 174CG | 79.66 |
| 95OG | 300C1 | 56CG | 49.06 |
| 95OG | 300C1 | 95CB | 14.32 |
| 174CG | 300C1 | 95CB | 73.86 |
| 56CG | 300C1 | 95CB | 63.06 |
| 174CD1 | 300C2 | 174CD2 | 37.67 |
| 174CD1 | 300C2 | 104OD2 | 60.60 |
| 174CD1 | 300C2 | 95OG | 60.29 |
| 174CD1 | 300C2 | 104OD1 | 73.12 |
| 174CD1 | 300C2 | 174CG | 19.40 |
| 174CD1 | 300C2 | 175CE1 | 60.61 |
| 174CD1 | 300C2 | 104CG | 61.72 |
| 174CD1 | 300C2 | 95CB | 55.80 |
| 174CD1 | 300C2 | 168NE2 | 62.72 |
| 174CD2 | 300C2 | 104OD2 | 90.84 |
| 174CD2 | 300C2 | 95OG | 84.48 |
| 174CD2 | 300C2 | 174CG | 19.96 |
| 174CD2 | 300C2 | 175CE1 | 41.70 |
| 174CD2 | 300C2 | 104CG | 96.88 |
| 174CD2 | 300C2 | 95CB | 87.57 |
| 174CD2 | 300C2 | 168NE2 | 72.77 |
| 104OD2 | 300C2 | 95OG | 81.21 |
| 104OD2 | 300C2 | 104OD1 | 29.39 |
| 104OD2 | 300C2 | 93OD1 | 68.62 |
| 104OD2 | 300C2 | 174CG | 71.81 |
| 104OD2 | 300C2 | 175CE1 | 77.23 |
| 104OD2 | 300C2 | 104CG | 15.46 |
| 104OD2 | 300C2 | 93OD2 | 72.94 |
| 104OD2 | 300C2 | 93CG | 76.26 |
| 104OD2 | 300C2 | 95CB | 64.77 |
| 104OD2 | 300C2 | 168NE2 | 36.99 |
| 95OG | 300C2 | 104OD1 | 62.76 |
| 95OG | 300C2 | 93OD1 | 65.66 |
| 95OG | 300C2 | 174CG | 76.06 |
| 95OG | 300C2 | 104CG | 67.82 |
| 95OG | 300C2 | 93OD2 | 93.20 |
| 95OG | 300C2 | 93CG | 78.95 |
| 95OG | 300C2 | 95CB | 16.63 |
| 104OD1 | 300C2 | 93OD1 | 41.49 |
| 104OD1 | 300C2 | 174CG | 90.16 |
| 104OD1 | 300C2 | 104CG | 15.52 |
| 104OD1 | 300C2 | 93OD2 | 55.34 |
| 104OD1 | 300C2 | 93CG | 52.55 |
| 104OD1 | 300C2 | 95CB | 46.89 |
| 104OD1 | 300C2 | 168NE2 | 66.01 |
| 93OD1 | 300C2 | 104CG | 56.86 |
| 93OD1 | 300C2 | 93OD2 | 27.59 |
| 93OD1 | 300C2 | 93CG | 14.94 |
| 93OD1 | 300C2 | 95CB | 58.87 |
| 174CG | 300C2 | 175CE1 | 44.45 |
| 174CG | 300C2 | 104CG | 76.95 |
| 174CG | 300C2 | 95CB | 74.28 |
| 174CG | 300C2 | 168NE2 | 60.83 |
| 175CE1 | 300C2 | 104CG | 91.08 |
| 175CE1 | 300C2 | 168NE2 | 43.13 |
| 175CE1 | 300C2 | 76CE1 | 92.75 |
| 104CG | 300C2 | 93OD2 | 67.53 |
| 104CG | 300C2 | 93CG | 67.06 |
| 104CG | 300C2 | 95CB | 51.19 |
| 104CG | 300C2 | 168NE2 | 52.35 |
| 93OD2 | 300C2 | 93CG | 15.23 |
| 93OD2 | 300C2 | 95CB | 85.84 |
| 93OD2 | 300C2 | 168NE2 | 93.24 |
| 93OD2 | 300C2 | 76CE1 | 79.01 |
| 93CG | 300C2 | 95CB | 73.55 |
| 93CG | 300C2 | 76CE1 | 86.79 |
| 95CB | 300C2 | 168NE2 | 96.40 |
| 67SG | 300C3 | 76CE1 | 79.93 |
| 67SG | 300C3 | 76NE2 | 87.30 |
| 67SG | 300C3 | 56CG | 64.01 |
| 67SG | 300C3 | 76ND1 | 63.97 |
| 67SG | 300C3 | 76CD2 | 75.35 |
| 76CE1 | 300C3 | 76NE2 | 17.93 |
| 76CE1 | 300C3 | 76ND1 | 16.05 |
| 76CE1 | 300C3 | 76CD2 | 27.36 |
| 76NE2 | 300C3 | 76ND1 | 27.26 |
| 76NE2 | 300C3 | 76CD2 | 16.26 |
| 174CD2 | 300C3 | 56CG | 97.93 |
| 76ND1 | 300C3 | 76CD2 | 26.71 |
| 174CD2 | 300C4 | 174CD1 | 47.78 |
| 174CD2 | 300C4 | 174CG | 23.77 |
| 174CD2 | 300C4 | 55CB | 95.90 |
| 174CD2 | 300C4 | 174CB | 29.29 |
| 174CD1 | 300C4 | 95OG | 78.45 |
| 174CD1 | 300C4 | 174CG | 24.16 |
| 174CD1 | 300C4 | 95CB | 65.13 |
| 174CD1 | 300C4 | 55CB | 71.48 |
| 174CD1 | 300C4 | 174CB | 30.61 |
| 174CD1 | 300C4 | 55C | 98.20 |
| 95OG | 300C4 | 56CG | 57.79 |
| 95OG | 300C4 | 95CB | 15.30 |
| 95OG | 300C4 | 56CD | 47.45 |
| 95OG | 300C4 | 56N | 62.45 |
| 95OG | 300C4 | 55CB | 45.17 |
| 95OG | 300C4 | 56CA | 78.07 |
| 95OG | 300C4 | 174CB | 96.24 |
| 95OG | 300C4 | 55C | 65.17 |
| 95OG | 300C4 | 56CB | 73.22 |
| 174CG | 300C4 | 95CB | 88.68 |
| 174CG | 300C4 | 55CB | 81.68 |
| 174CG | 300C4 | 174CB | 16.66 |
| 174CG | 300C4 | 55C | 99.87 |
| 56CG | 300C4 | 95CB | 73.01 |
| 56CG | 300C4 | 56CD | 19.80 |
| 56CG | 300C4 | 56N | 30.94 |
| 56CG | 300C4 | 55CB | 63.24 |
| 56CG | 300C4 | 56CA | 30.65 |
| 56CG | 300C4 | 55C | 47.01 |
| 56CG | 300C4 | 56CB | 16.62 |
| 95CB | 300C4 | 56CD | 62.34 |
| 95CB | 300C4 | 56N | 76.04 |
| 95CB | 300C4 | 55CB | 50.54 |
| 95CB | 300C4 | 56CA | 92.61 |
| 95CB | 300C4 | 174CB | 87.06 |
| 95CB | 300C4 | 55C | 76.06 |
| 95CB | 300C4 | 56CB | 88.52 |
| 56CD | 300C4 | 56N | 18.86 |
| 56CD | 300C4 | 55CB | 43.44 |
| 56CD | 300C4 | 56CA | 30.79 |
| 56CD | 300C4 | 55C | 31.69 |
| 56CD | 300C4 | 56CB | 28.60 |
| 56N | 300C4 | 55CB | 41.27 |
| 56N | 300C4 | 56CA | 18.42 |
| 56N | 300C4 | 174CB | 99.66 |
| 56N | 300C4 | 55C | 16.30 |
| 56N | 300C4 | 56CB | 28.51 |
| 55CB | 300C4 | 56CA | 59.00 |
| 55CB | 300C4 | 174CB | 68.02 |
| 55CB | 300C4 | 55C | 31.10 |
| 55CB | 300C4 | 56CB | 68.44 |
| 56CA | 300C4 | 55C | 29.74 |
| 56CA | 300C4 | 56CB | 18.26 |
| 174CB | 300C4 | 55C | 83.39 |
| 55C | 300C4 | 56CB | 44.03 |
| 175CE1 | 300C5 | 174CD1 | 74.10 |
| 175CE1 | 300C5 | 168NE2 | 58.91 |
| 175CE1 | 300C5 | 174CD2 | 49.97 |
| 175CE1 | 300C5 | 174CG | 53.35 |
| 175CE1 | 300C5 | 168CE1 | 57.33 |
| 175CE1 | 300C5 | 175NE2 | 14.62 |
| 175CE1 | 300C5 | 175ND1 | 13.75 |
| 175CE1 | 300C5 | 168CD2 | 50.49 |
| 175CE1 | 300C5 | 202OE1 | 86.19 |
| 174CD1 | 300C5 | 104OD2 | 69.77 |
| 174CD1 | 300C5 | 168NE2 | 80.34 |
| 174CD1 | 300C5 | 174CD2 | 40.03 |
| 174CD1 | 300C5 | 174CG | 22.15 |
| 174CD1 | 300C5 | 168CE1 | 63.75 |
| 174CD1 | 300C5 | 104CG | 67.11 |
| 174CD1 | 300C5 | 104OD1 | 75.50 |
| 174CD1 | 300C5 | 175NE2 | 87.86 |
| 174CD1 | 300C5 | 175ND1 | 60.36 |
| 174CD1 | 300C5 | 168CD2 | 89.95 |
| 104OD2 | 300C5 | 168NE2 | 48.89 |
| 104OD2 | 300C5 | 174CG | 84.33 |
| 104OD2 | 300C5 | 168CE1 | 45.07 |
| 104OD2 | 300C5 | 104CG | 15.33 |
| 104OD2 | 300C5 | 104OD1 | 30.53 |
| 104OD2 | 300C5 | 175ND1 | 96.64 |
| 104OD2 | 300C5 | 168CD2 | 63.15 |
| 104OD2 | 300C5 | 202OE1 | 62.28 |
| 104OD2 | 300C5 | 233OE2 | 33.68 |
| 168NE2 | 300C5 | 174CD2 | 92.51 |
| 168NE2 | 300C5 | 174CG | 76.98 |
| 168NE2 | 300C5 | 168CE1 | 16.60 |
| 168NE2 | 300C5 | 104CG | 64.09 |
| 168NE2 | 300C5 | 104OD1 | 79.05 |
| 168NE2 | 300C5 | 175NE2 | 55.42 |
| 168NE2 | 300C5 | 175ND1 | 60.08 |
| 168NE2 | 300C5 | 168CD2 | 15.06 |
| 168NE2 | 300C5 | 202OE1 | 44.18 |
| 168NE2 | 300C5 | 233OE2 | 56.34 |
| 174CD2 | 300C5 | 174CG | 22.17 |
| 174CD2 | 300C5 | 168CE1 | 79.92 |
| 174CD2 | 300C5 | 175NE2 | 64.07 |
| 174CD2 | 300C5 | 175ND1 | 39.17 |
| 174CD2 | 300C5 | 168CD2 | 92.61 |
| 174CG | 300C5 | 168CE1 | 62.02 |
| 174CG | 300C5 | 104CG | 85.66 |
| 174CG | 300C5 | 104OD1 | 96.42 |
| 174CG | 300C5 | 175NE2 | 67.72 |
| 174CG | 300C5 | 175ND1 | 39.70 |
| 174CG | 300C5 | 168CD2 | 81.66 |
| 168CE1 | 300C5 | 104CG | 58.70 |
| 168CE1 | 300C5 | 104OD1 | 75.16 |
| 168CE1 | 300C5 | 175NE2 | 58.81 |
| 168CE1 | 300C5 | 175ND1 | 53.96 |
| 168CE1 | 300C5 | 168CD2 | 28.78 |
| 168CE1 | 300C5 | 202OE1 | 59.99 |
| 168CE1 | 300C5 | 233OE2 | 63.52 |
| 104CG | 300C5 | 104OD1 | 16.67 |
| 104CG | 300C5 | 168CD2 | 78.47 |
| 104CG | 300C5 | 202OE1 | 73.75 |
| 104CG | 300C5 | 233OE2 | 38.78 |
| 104OD1 | 300C5 | 168CD2 | 92.63 |
| 104OD1 | 300C5 | 202OE1 | 78.88 |
| 104OD1 | 300C5 | 233OE2 | 40.21 |
| 175NE2 | 300C5 | 175ND1 | 28.03 |
| 175NE2 | 300C5 | 168CD2 | 43.66 |
| 175NE2 | 300C5 | 202OE1 | 74.27 |
| 175ND1 | 300C5 | 168CD2 | 55.46 |
| 175ND1 | 300C5 | 202OE1 | 94.63 |
| 168CD2 | 300C5 | 202OE1 | 40.71 |
| 168CD2 | 300C5 | 233OE2 | 64.23 |
| 202OE1 | 300C5 | 233OE2 | 38.87 |
| 93OD1 | 300N6 | 93OD2 | 39.13 |
| 93OD1 | 300N6 | 93CG | 21.29 |
| 93OD1 | 300N6 | 104OD1 | 54.47 |
| 93OD1 | 300N6 | 104OD2 | 85.29 |
| 93OD1 | 300N6 | 104CG | 70.65 |
| 93OD1 | 300N6 | 95OG | 76.44 |
| 93OD1 | 300N6 | 233OE1 | 44.59 |
| 93OD1 | 300N6 | 93CB | 29.20 |
| 93OD1 | 300N6 | 95CB | 68.18 |
| 93OD1 | 300N6 | 233OE2 | 70.83 |
| 93OD2 | 300N6 | 93CG | 21.29 |
| 93OD2 | 300N6 | 104OD1 | 74.41 |
| 93OD2 | 300N6 | 104OD2 | 92.03 |
| 93OD2 | 300N6 | 104CG | 86.14 |
| 93OD2 | 300N6 | 233OE1 | 37.50 |
| 93OD2 | 300N6 | 93CB | 30.89 |
| 93OD2 | 300N6 | 233OE2 | 60.37 |
| 93CG | 300N6 | 104OD1 | 70.57 |
| 93CG | 300N6 | 104OD2 | 97.02 |
| 93CG | 300N6 | 104CG | 85.64 |
| 93CG | 300N6 | 95OG | 95.35 |
| 93CG | 300N6 | 233OE1 | 45.40 |
| 93CG | 300N6 | 93CB | 14.73 |
| 93CG | 300N6 | 95CB | 89.07 |
| 93CG | 300N6 | 233OE2 | 72.37 |
| 104OD1 | 300N6 | 104OD2 | 32.66 |
| 104OD1 | 300N6 | 104CG | 16.29 |
| 104OD1 | 300N6 | 95OG | 68.37 |
| 104OD1 | 300N6 | 233OE1 | 41.24 |
| 104OD1 | 300N6 | 93CB | 82.86 |
| 104OD1 | 300N6 | 174CD1 | 71.17 |
| 104OD1 | 300N6 | 95CB | 50.82 |
| 104OD1 | 300N6 | 233OE2 | 42.33 |
| 104OD2 | 300N6 | 104CG | 17.34 |
| 104OD2 | 300N6 | 95OG | 83.69 |
| 104OD2 | 300N6 | 233OE1 | 54.67 |
| 104OD2 | 300N6 | 174CD1 | 55.92 |
| 104OD2 | 300N6 | 95CB | 68.14 |
| 104OD2 | 300N6 | 233OE2 | 35.07 |
| 104CG | 300N6 | 95OG | 71.74 |
| 104CG | 300N6 | 233OE1 | 49.60 |
| 104CG | 300N6 | 93CB | 98.62 |
| 104CG | 300N6 | 174CD1 | 59.32 |
| 104CG | 300N6 | 95CB | 54.76 |
| 104CG | 300N6 | 233OE2 | 39.82 |
| 95OG | 300N6 | 93CB | 92.79 |
| 95OG | 300N6 | 174CD1 | 54.13 |
| 95OG | 300N6 | 95CB | 17.58 |
| 233OE1 | 300N6 | 93CB | 60.09 |
| 233OE1 | 300N6 | 95CB | 87.80 |
| 233OE1 | 300N6 | 233OE2 | 27.34 |
| 93CB | 300N6 | 95CB | 90.88 |
| 93CB | 300N6 | 233OE2 | 86.89 |
| 174CD1 | 300N6 | 95CB | 52.74 |
| 174CD1 | 300N6 | 233OE2 | 90.57 |
| 95CB | 300N6 | 233OE2 | 92.67 |
| 56CG | 300C7 | 56CB | 21.05 |
| 56CG | 300C7 | 67SG | 69.49 |
| 56CG | 300C7 | 56CA | 32.59 |
| 56CB | 300C7 | 67SG | 73.79 |
| 56CB | 300C7 | 60CG | 85.83 |
| 56CB | 300C7 | 56CA | 20.77 |
| 67SG | 300C7 | 56CA | 94.55 |
| 67SG | 300C7 | 76NE2 | 81.09 |
| 67SG | 300C7 | 76CD2 | 73.41 |
| 60CG | 300C7 | 56CA | 68.06 |
| 60CG | 300C7 | 174CD2 | 69.46 |
| 56CA | 300C7 | 174CD2 | 92.98 |
| 76NE2 | 300C7 | 174CD2 | 97.90 |
| 76NE2 | 300C7 | 76CD2 | 16.08 |
| 56CA | 300C8 | 56CG | 40.13 |
| 56CA | 300C8 | 56N | 24.43 |
| 56CA | 300C8 | 55C | 38.85 |
| 56CA | 300C8 | 95OG | 89.78 |
| 56CA | 300C8 | 56CD | 38.52 |
| 56CA | 300C8 | 56CB | 23.00 |
| 56CA | 300C8 | 55O | 42.98 |
| 56CA | 300C8 | 60CG | 78.72 |
| 56CA | 300C8 | 55CB | 71.18 |
| 56CA | 300C8 | 56C | 12.37 |
| 56CA | 300C8 | 55CA | 53.45 |
| 56CA | 300C8 | 56O | 26.21 |
| 56CA | 300C8 | 60OE2 | 96.90 |
| 56CG | 300C8 | 56N | 39.05 |
| 56CG | 300C8 | 55C | 59.14 |
| 56CG | 300C8 | 95OG | 59.87 |
| 56CG | 300C8 | 56CD | 23.38 |
| 56CG | 300C8 | 56CB | 22.71 |
| 56CG | 300C8 | 55O | 73.69 |
| 56CG | 300C8 | 55CB | 71.16 |
| 56CG | 300C8 | 56C | 52.31 |
| 56CG | 300C8 | 55CA | 62.94 |
| 56CG | 300C8 | 56O | 64.61 |
| 56N | 300C8 | 55C | 20.74 |
| 56N | 300C8 | 95OG | 69.52 |
| 56N | 300C8 | 56CD | 22.66 |
| 56N | 300C8 | 56CB | 36.48 |
| 56N | 300C8 | 55O | 34.91 |
| 56N | 300C8 | 60CG | 96.57 |
| 56N | 300C8 | 55CB | 47.25 |
| 56N | 300C8 | 56C | 32.19 |
| 56N | 300C8 | 55CA | 30.81 |
| 56N | 300C8 | 56O | 46.18 |
| 174CD2 | 300C8 | 95OG | 99.28 |
| 174CD2 | 300C8 | 60CG | 85.76 |
| 174CD2 | 300C8 | 174CD1 | 37.45 |
| 174CD2 | 300C8 | 55CB | 93.73 |
| 174CD2 | 300C8 | 174CG | 18.45 |
| 174CD2 | 300C8 | 60OE2 | 60.17 |
| 174CD2 | 300C8 | 174CB | 27.49 |
| 55C | 300C8 | 95OG | 72.99 |
| 55C | 300C8 | 56CD | 39.00 |
| 55C | 300C8 | 56CB | 56.33 |
| 55C | 300C8 | 55O | 18.39 |
| 55C | 300C8 | 60CG | 91.45 |
| 55C | 300C8 | 55CB | 35.64 |
| 55C | 300C8 | 56C | 40.40 |
| 55C | 300C8 | 55CA | 16.83 |
| 55C | 300C8 | 56O | 50.68 |
| 55C | 300C8 | 60OE2 | 92.17 |
| 55C | 300C8 | 174CB | 89.79 |
| 95OG | 300C8 | 56CD | 51.29 |
| 95OG | 300C8 | 56CB | 81.74 |
| 95OG | 300C8 | 55O | 89.88 |
| 95OG | 300C8 | 174CD1 | 61.90 |
| 95OG | 300C8 | 55CB | 46.59 |
| 95OG | 300C8 | 174CG | 81.81 |
| 95OG | 300C8 | 55CA | 59.32 |
| 95OG | 300C8 | 174CB | 84.18 |
| 56CD | 300C8 | 56CB | 35.36 |
| 56CD | 300C8 | 55O | 56.17 |
| 56CD | 300C8 | 55CB | 48.33 |
| 56CD | 300C8 | 56C | 49.90 |
| 56CD | 300C8 | 55CA | 39.81 |
| 56CD | 300C8 | 56O | 64.35 |
| 56CB | 300C8 | 55O | 64.88 |
| 56CB | 300C8 | 60CG | 91.22 |
| 56CB | 300C8 | 55CB | 81.07 |
| 56CB | 300C8 | 56C | 33.27 |
| 56CB | 300C8 | 55CA | 66.90 |
| 56CB | 300C8 | 56O | 43.35 |
| 55O | 300C8 | 60CG | 74.46 |
| 55O | 300C8 | 55CB | 47.15 |
| 55O | 300C8 | 56C | 38.47 |
| 55O | 300C8 | 55CA | 30.73 |
| 55O | 300C8 | 56O | 43.20 |
| 55O | 300C8 | 60OE2 | 73.89 |
| 55O | 300C8 | 174CB | 84.19 |
| 60CG | 300C8 | 56C | 66.93 |
| 60CG | 300C8 | 56O | 52.56 |
| 60CG | 300C8 | 60OE2 | 28.38 |
| 60CG | 300C8 | 174CB | 94.18 |
| 174CD1 | 300C8 | 55CB | 67.01 |
| 174CD1 | 300C8 | 174CG | 20.20 |
| 174CD1 | 300C8 | 55CA | 84.90 |
| 174CD1 | 300C8 | 60OE2 | 93.20 |
| 174CD1 | 300C8 | 174CB | 29.70 |
| 55CB | 300C8 | 174CG | 77.41 |
| 55CB | 300C8 | 56C | 75.50 |
| 55CB | 300C8 | 55CA | 18.83 |
| 55CB | 300C8 | 56O | 86.30 |
| 55CB | 300C8 | 174CB | 66.76 |
| 174CG | 300C8 | 55CA | 92.54 |
| 174CG | 300C8 | 60OE2 | 73.50 |
| 174CG | 300C8 | 174CB | 17.14 |
| 56C | 300C8 | 55CA | 56.79 |
| 56C | 300C8 | 56O | 14.49 |
| 56C | 300C8 | 60OE2 | 84.57 |
| 55CA | 300C8 | 56O | 67.51 |
| 55CA | 300C8 | 60OE2 | 99.40 |
| 55CA | 300C8 | 174CB | 79.13 |
| 56O | 300C8 | 60OE2 | 72.09 |
| 60OE2 | 300C8 | 174CB | 65.82 |
| 104OD2 | 300N9 | 168NE2 | 58.52 |
| 104OD2 | 300N9 | 233OE2 | 48.91 |
| 104OD2 | 300N9 | 104OD1 | 37.49 |
| 104OD2 | 300N9 | 104CG | 17.98 |
| 104OD2 | 300N9 | 202OE1 | 84.22 |
| 104OD2 | 300N9 | 168CE1 | 48.93 |
| 104OD2 | 300N9 | 202OE2 | 93.99 |
| 104OD2 | 300N9 | 168CD2 | 73.29 |
| 104OD2 | 300N9 | 93OD2 | 96.73 |
| 104OD2 | 300N9 | 202CD | 85.15 |
| 104OD2 | 300N9 | 175CE1 | 99.37 |
| 104OD2 | 300N9 | 233OE1 | 62.73 |
| 104OD2 | 300N9 | 233CD | 53.68 |
| 104OD2 | 300N9 | 174CD1 | 65.29 |
| 104OD2 | 300N9 | 93OD1 | 80.47 |
| 104OD2 | 300N9 | 93CG | 91.94 |
| 168NE2 | 300N9 | 233OE2 | 75.46 |
| 168NE2 | 300N9 | 104OD1 | 95.99 |
| 168NE2 | 300N9 | 104CG | 76.38 |
| 168NE2 | 300N9 | 202OE1 | 57.24 |
| 168NE2 | 300N9 | 168CE1 | 15.65 |
| 168NE2 | 300N9 | 202OE2 | 87.80 |
| 168NE2 | 300N9 | 168CD2 | 15.93 |
| 168NE2 | 300N9 | 202CD | 71.02 |
| 168NE2 | 300N9 | 175CE1 | 53.26 |
| 168NE2 | 300N9 | 233CD | 88.90 |
| 168NE2 | 300N9 | 174CD1 | 73.59 |
| 168NE2 | 300N9 | 175NE2 | 49.96 |
| 233OE2 | 300N9 | 104OD1 | 53.02 |
| 233OE2 | 300N9 | 104CG | 51.03 |
| 233OE2 | 300N9 | 202OE1 | 52.33 |
| 233OE2 | 300N9 | 168CE1 | 78.24 |
| 233OE2 | 300N9 | 202OE2 | 46.67 |
| 233OE2 | 300N9 | 168CD2 | 80.31 |
| 233OE2 | 300N9 | 93OD2 | 65.76 |
| 233OE2 | 300N9 | 202CD | 43.83 |
| 233OE2 | 300N9 | 233OE1 | 30.45 |
| 233OE2 | 300N9 | 233CD | 13.88 |
| 233OE2 | 300N9 | 93OD1 | 71.25 |
| 233OE2 | 300N9 | 93CG | 71.58 |
| 104OD1 | 300N9 | 104CG | 19.91 |
| 104OD1 | 300N9 | 168CE1 | 85.68 |
| 104OD1 | 300N9 | 202OE2 | 95.12 |
| 104OD1 | 300N9 | 93OD2 | 64.70 |
| 104OD1 | 300N9 | 202CD | 96.62 |
| 104OD1 | 300N9 | 233OE1 | 42.80 |
| 104OD1 | 300N9 | 233CD | 46.29 |
| 104OD1 | 300N9 | 174CD1 | 74.43 |
| 104OD1 | 300N9 | 93OD1 | 43.31 |
| 104OD1 | 300N9 | 93CG | 56.23 |
| 104CG | 300N9 | 202OE1 | 96.40 |
| 104CG | 300N9 | 168CE1 | 65.79 |
| 104CG | 300N9 | 202OE2 | 97.58 |
| 104CG | 300N9 | 168CD2 | 91.26 |
| 104CG | 300N9 | 93OD2 | 82.85 |
| 104CG | 300N9 | 202CD | 93.29 |
| 104CG | 300N9 | 233OE1 | 53.88 |
| 104CG | 300N9 | 233CD | 50.29 |
| 104CG | 300N9 | 174CD1 | 65.79 |
| 104CG | 300N9 | 93OD1 | 63.22 |
| 104CG | 300N9 | 93CG | 75.78 |
| 202OE1 | 300N9 | 168CE1 | 71.36 |
| 202OE1 | 300N9 | 202OE2 | 31.63 |
| 202OE1 | 300N9 | 168CD2 | 48.43 |
| 202OE1 | 300N9 | 93OD2 | 93.39 |
| 202OE1 | 300N9 | 202CD | 15.80 |
| 202OE1 | 300N9 | 175CE1 | 91.49 |
| 202OE1 | 300N9 | 233OE1 | 75.39 |
| 202OE1 | 300N9 | 233CD | 62.68 |
| 202OE1 | 300N9 | 175NE2 | 78.47 |
| 168CE1 | 300N9 | 168CD2 | 30.86 |
| 168CE1 | 300N9 | 202CD | 83.87 |
| 168CE1 | 300N9 | 175CE1 | 53.79 |
| 168CE1 | 300N9 | 233CD | 90.25 |
| 168CE1 | 300N9 | 174CD1 | 59.28 |
| 168CE1 | 300N9 | 175NE2 | 55.30 |
| 202OE2 | 300N9 | 168CD2 | 80.06 |
| 202OE2 | 300N9 | 93OD2 | 62.37 |
| 202OE2 | 300N9 | 202CD | 16.78 |
| 202OE2 | 300N9 | 233OE1 | 54.57 |
| 202OE2 | 300N9 | 233CD | 49.09 |
| 202OE2 | 300N9 | 93OD1 | 87.36 |
| 202OE2 | 300N9 | 93CG | 76.49 |
| 168CD2 | 300N9 | 202CD | 63.81 |
| 168CD2 | 300N9 | 175CE1 | 49.31 |
| 168CD2 | 300N9 | 233CD | 94.18 |
| 168CD2 | 300N9 | 174CD1 | 84.70 |
| 168CD2 | 300N9 | 175NE2 | 41.49 |
| 93OD2 | 300N9 | 202CD | 77.62 |
| 93OD2 | 300N9 | 233OE1 | 37.44 |
| 93OD2 | 300N9 | 233CD | 52.80 |
| 93OD2 | 300N9 | 93OD1 | 28.93 |
| 93OD2 | 300N9 | 93CG | 14.72 |
| 202CD | 300N9 | 233OE1 | 61.82 |
| 202CD | 300N9 | 233CD | 51.34 |
| 202CD | 300N9 | 93OD1 | 99.92 |
| 202CD | 300N9 | 93CG | 91.03 |
| 202CD | 300N9 | 175NE2 | 93.50 |
| 175CE1 | 300N9 | 174CD1 | 59.08 |
| 175CE1 | 300N9 | 175NE2 | 14.43 |
| 233OE1 | 300N9 | 233CD | 16.58 |
| 233OE1 | 300N9 | 93OD1 | 41.84 |
| 233OE1 | 300N9 | 93CG | 41.20 |
| 233CD | 300N9 | 93OD1 | 57.65 |
| 233CD | 300N9 | 93CG | 57.76 |
| 174CD1 | 300N9 | 93OD1 | 97.74 |
| 174CD1 | 300N9 | 175NE2 | 72.98 |
| 93OD1 | 300N9 | 93CG | 15.23 |
| 93OD2 | 300N10 | 104OD1 | 87.73 |
| 93OD2 | 300N10 | 93OD1 | 41.00 |
| 93OD2 | 300N10 | 93CG | 21.28 |
| 93OD2 | 300N10 | 233OE2 | 81.90 |
| 93OD2 | 300N10 | 233OE1 | 48.95 |
| 93OD2 | 300N10 | 233CD | 65.46 |
| 93OD2 | 300N10 | 202OE2 | 72.75 |
| 93OD2 | 300N10 | 202CD | 86.24 |
| 93OD2 | 300N10 | 93CB | 24.15 |
| 104OD1 | 300N10 | 104OD2 | 40.61 |
| 104OD1 | 300N10 | 93OD1 | 58.72 |
| 104OD1 | 300N10 | 93CG | 76.82 |
| 104OD1 | 300N10 | 233OE2 | 57.98 |
| 104OD1 | 300N10 | 233OE1 | 52.89 |
| 104OD1 | 300N10 | 104CG | 20.23 |
| 104OD1 | 300N10 | 233CD | 52.94 |
| 104OD1 | 300N10 | 168NE2 | 83.47 |
| 104OD1 | 300N10 | 202CD | 98.53 |
| 104OD1 | 300N10 | 93CB | 84.28 |
| 104OD1 | 300N10 | 174CD1 | 70.14 |
| 104OD2 | 300N10 | 93OD1 | 99.13 |
| 104OD2 | 300N10 | 233OE2 | 47.97 |
| 104OD2 | 300N10 | 233OE1 | 71.38 |
| 104OD2 | 300N10 | 104CG | 20.56 |
| 104OD2 | 300N10 | 233CD | 58.20 |
| 104OD2 | 300N10 | 202OE2 | 88.90 |
| 104OD2 | 300N10 | 168NE2 | 42.87 |
| 104OD2 | 300N10 | 202OE1 | 70.13 |
| 104OD2 | 300N10 | 202CD | 76.28 |
| 104OD2 | 300N10 | 174CD1 | 55.61 |
| 93OD1 | 300N10 | 93CG | 21.32 |
| 93OD1 | 300N10 | 233OE2 | 90.16 |
| 93OD1 | 300N10 | 233OE1 | 55.45 |
| 93OD1 | 300N10 | 104CG | 78.95 |
| 93OD1 | 300N10 | 233CD | 72.70 |
| 93OD1 | 300N10 | 93CB | 26.33 |
| 93CG | 300N10 | 233OE2 | 91.55 |
| 93CG | 300N10 | 233OE1 | 55.04 |
| 93CG | 300N10 | 104CG | 96.56 |
| 93CG | 300N10 | 233CD | 73.46 |
| 93CG | 300N10 | 202OE2 | 93.21 |
| 93CG | 300N10 | 93CB | 9.22 |
| 233OE2 | 300N10 | 233OE1 | 36.62 |
| 233OE2 | 300N10 | 104CG | 52.20 |
| 233OE2 | 300N10 | 233CD | 18.18 |
| 233OE2 | 300N10 | 202OE2 | 47.00 |
| 233OE2 | 300N10 | 168NE2 | 62.41 |
| 233OE2 | 300N10 | 202OE1 | 46.54 |
| 233OE2 | 300N10 | 202CD | 40.73 |
| 233OE1 | 300N10 | 104CG | 62.21 |
| 233OE1 | 300N10 | 233CD | 18.45 |
| 233OE1 | 300N10 | 202OE2 | 60.34 |
| 233OE1 | 300N10 | 168NE2 | 98.51 |
| 233OE1 | 300N10 | 202OE1 | 76.57 |
| 233OE1 | 300N10 | 202CD | 64.29 |
| 233OE1 | 300N10 | 93CB | 63.89 |
| 104CG | 300N10 | 233CD | 54.91 |
| 104CG | 300N10 | 202OE2 | 98.68 |
| 104CG | 300N10 | 168NE2 | 63.34 |
| 104CG | 300N10 | 202OE1 | 86.64 |
| 104CG | 300N10 | 202CD | 89.02 |
| 104CG | 300N10 | 174CD1 | 59.40 |
| 233CD | 300N10 | 202OE2 | 52.05 |
| 233CD | 300N10 | 168NE2 | 80.24 |
| 233CD | 300N10 | 202OE1 | 61.15 |
| 233CD | 300N10 | 202CD | 51.37 |
| 233CD | 300N10 | 93CB | 82.23 |
| 202OE2 | 300N10 | 168NE2 | 74.00 |
| 202OE2 | 300N10 | 202OE1 | 29.35 |
| 202OE2 | 300N10 | 202CD | 14.98 |
| 202OE2 | 300N10 | 93CB | 96.76 |
| 168NE2 | 300N10 | 202OE1 | 45.02 |
| 168NE2 | 300N10 | 202CD | 59.20 |
| 168NE2 | 300N10 | 174CD1 | 59.19 |
| 202OE1 | 300N10 | 202CD | 15.53 |
| 56CA | 300C11 | 56CB | 26.44 |
| 56CA | 300C11 | 56CG | 41.44 |
| 56CA | 300C11 | 60CG | 91.21 |
| 56CA | 300C11 | 56N | 21.20 |
| 56CA | 300C11 | 56CD | 34.51 |
| 56CA | 300C11 | 56C | 16.30 |
| 56CA | 300C11 | 60CB | 78.90 |
| 56CA | 300C11 | 56O | 32.73 |
| 56CA | 300C11 | 55O | 37.53 |
| 56CA | 300C11 | 55C | 32.06 |
| 56CA | 300C11 | 60CD | 99.64 |
| 56CA | 300C11 | 95OG | 75.60 |
| 56CA | 300C11 | 62CB | 54.27 |
| 56CA | 300C11 | 60OE2 | 99.48 |
| 56CB | 300C11 | 56CG | 25.19 |
| 56CB | 300C11 | 56N | 36.84 |
| 56CB | 300C11 | 56CD | 33.62 |
| 56CB | 300C11 | 56C | 36.50 |
| 56CB | 300C11 | 60CB | 94.13 |
| 56CB | 300C11 | 56O | 49.89 |
| 56CB | 300C11 | 55O | 62.84 |
| 56CB | 300C11 | 55C | 53.20 |
| 56CB | 300C11 | 95OG | 72.76 |
| 56CB | 300C11 | 62CB | 47.79 |
| 56CG | 300C11 | 56N | 36.80 |
| 56CG | 300C11 | 56CD | 19.74 |
| 56CG | 300C11 | 56C | 56.45 |
| 56CG | 300C11 | 56O | 72.08 |
| 56CG | 300C11 | 55O | 67.51 |
| 56CG | 300C11 | 55C | 52.80 |
| 56CG | 300C11 | 95OG | 48.84 |
| 56CG | 300C11 | 62CB | 71.64 |
| 60CG | 300C11 | 56C | 76.14 |
| 60CG | 300C11 | 60CB | 19.01 |
| 60CG | 300C11 | 56O | 61.01 |
| 60CG | 300C11 | 55O | 74.48 |
| 60CG | 300C11 | 55C | 90.43 |
| 60CG | 300C11 | 174CD2 | 80.87 |
| 60CG | 300C11 | 60CD | 12.94 |
| 60CG | 300C11 | 62CB | 76.93 |
| 60CG | 300C11 | 60OE2 | 25.79 |
| 56N | 300C11 | 56CD | 20.41 |
| 56N | 300C11 | 56C | 35.34 |
| 56N | 300C11 | 60CB | 93.75 |
| 56N | 300C11 | 56O | 50.73 |
| 56N | 300C11 | 55O | 30.72 |
| 56N | 300C11 | 55C | 17.05 |
| 56N | 300C11 | 95OG | 56.20 |
| 56N | 300C11 | 62CB | 75.09 |
| 56CD | 300C11 | 56C | 50.78 |
| 56CD | 300C11 | 56O | 67.17 |
| 56CD | 300C11 | 55O | 49.81 |
| 56CD | 300C11 | 55C | 34.20 |
| 56CD | 300C11 | 95OG | 42.17 |
| 56CD | 300C11 | 62CB | 80.55 |
| 56C | 300C11 | 60CB | 62.76 |
| 56C | 300C11 | 56O | 16.43 |
| 56C | 300C11 | 55O | 38.92 |
| 56C | 300C11 | 55C | 40.55 |
| 56C | 300C11 | 60CD | 85.77 |
| 56C | 300C11 | 95OG | 91.25 |
| 56C | 300C11 | 62CB | 45.19 |
| 56C | 300C11 | 60OE2 | 88.19 |
| 60CB | 300C11 | 56O | 46.50 |
| 60CB | 300C11 | 55O | 73.15 |
| 60CB | 300C11 | 55C | 87.59 |
| 60CB | 300C11 | 174CD2 | 99.62 |
| 60CB | 300C11 | 60CD | 31.95 |
| 60CB | 300C11 | 62CB | 57.96 |
| 60CB | 300C11 | 60OE2 | 44.01 |
| 56O | 300C11 | 55O | 45.36 |
| 56O | 300C11 | 55C | 52.24 |
| 56O | 300C11 | 60CD | 71.73 |
| 56O | 300C11 | 62CB | 40.02 |
| 56O | 300C11 | 60OE2 | 76.67 |
| 55O | 300C11 | 55C | 16.06 |
| 55O | 300C11 | 174CD2 | 84.94 |
| 55O | 300C11 | 60CD | 76.44 |
| 55O | 300C11 | 95OG | 72.01 |
| 55O | 300C11 | 62CB | 83.51 |
| 55O | 300C11 | 60OE2 | 69.80 |
| 55C | 300C11 | 174CD2 | 89.26 |
| 55C | 300C11 | 60CD | 92.42 |
| 55C | 300C11 | 95OG | 58.36 |
| 55C | 300C11 | 62CB | 85.09 |
| 55C | 300C11 | 60OE2 | 84.99 |
| 174CD2 | 300C11 | 60CD | 68.13 |
| 174CD2 | 300C11 | 95OG | 74.23 |
| 174CD2 | 300C11 | 60OE2 | 55.70 |
| 60CD | 300C11 | 62CB | 89.84 |
| 60CD | 300C11 | 60OE2 | 14.49 |
| 62CD1 | 300I12 | 67SG | 82.42 |
| 62CD1 | 300I12 | 62CG | 20.77 |
| 62CD1 | 300I12 | 62CE1 | 20.60 |
| 62CD1 | 300I12 | 219CZ3 | 61.51 |
| 62CD1 | 300I12 | 219CE3 | 54.62 |
| 62CD1 | 300I12 | 62CB | 36.28 |
| 62CD1 | 300I12 | 62CD2 | 30.68 |
| 62CD1 | 300I12 | 62CZ | 30.61 |
| 62CD1 | 300I12 | 56CB | 74.90 |
| 62CD1 | 300I12 | 76CB | 87.89 |
| 62CD1 | 300I12 | 60CB | 85.12 |
| 62CD1 | 300I12 | 62CE2 | 33.19 |
| 62CD1 | 300I12 | 219CH2 | 55.86 |
| 67SG | 300I12 | 62CG | 78.26 |
| 67SG | 300I12 | 62CE1 | 68.36 |
| 67SG | 300I12 | 76CD2 | 89.86 |
| 67SG | 300I12 | 76CG | 72.01 |
| 67SG | 300I12 | 62CB | 93.65 |
| 67SG | 300I12 | 76NE2 | 90.83 |
| 67SG | 300I12 | 62CD2 | 62.38 |
| 67SG | 300I12 | 62CZ | 53.10 |
| 67SG | 300I12 | 56CB | 70.74 |
| 67SG | 300I12 | 76CB | 65.38 |
| 67SG | 300I12 | 76ND1 | 65.19 |
| 67SG | 300I12 | 76CE1 | 76.13 |
| 67SG | 300I12 | 62CE2 | 50.70 |
| 62CG | 300I12 | 62CE1 | 36.01 |
| 62CG | 300I12 | 219CZ3 | 74.48 |
| 62CG | 300I12 | 219CE3 | 72.59 |
| 62CG | 300I12 | 62CB | 20.71 |
| 62CG | 300I12 | 62CD2 | 16.29 |
| 62CG | 300I12 | 60CG | 94.06 |
| 62CG | 300I12 | 62CZ | 37.02 |
| 62CG | 300I12 | 56CB | 54.22 |
| 62CG | 300I12 | 60CB | 78.11 |
| 62CG | 300I12 | 62CE2 | 28.90 |
| 62CG | 300I12 | 219CH2 | 65.31 |
| 62CE1 | 300I12 | 76CD2 | 97.74 |
| 62CE1 | 300I12 | 219CZ3 | 70.98 |
| 62CE1 | 300I12 | 219CE3 | 57.53 |
| 62CE1 | 300I12 | 76CG | 86.56 |
| 62CE1 | 300I12 | 62CB | 55.22 |
| 62CE1 | 300I12 | 62CD2 | 36.83 |
| 62CE1 | 300I12 | 62CZ | 16.13 |
| 62CE1 | 300I12 | 56CB | 84.96 |
| 62CE1 | 300I12 | 76CB | 68.61 |
| 62CE1 | 300I12 | 76ND1 | 97.18 |
| 62CE1 | 300I12 | 62CE2 | 28.66 |
| 62CE1 | 300I12 | 219CH2 | 69.39 |
| 76CD2 | 300I12 | 219CZ3 | 86.59 |
| 76CD2 | 300I12 | 219CE3 | 75.63 |
| 76CD2 | 300I12 | 76CG | 18.49 |
| 76CD2 | 300I12 | 76NE2 | 18.34 |
| 76CD2 | 300I12 | 76CB | 32.99 |
| 76CD2 | 300I12 | 76ND1 | 26.87 |
| 76CD2 | 300I12 | 76CE1 | 26.69 |
| 76CD2 | 300I12 | 219CH2 | 99.45 |
| 219CZ3 | 300I12 | 219CE3 | 19.81 |
| 219CZ3 | 300I12 | 76CG | 96.82 |
| 219CZ3 | 300I12 | 62CB | 70.12 |
| 219CZ3 | 300I12 | 76NE2 | 98.01 |
| 219CZ3 | 300I12 | 62CD2 | 89.88 |
| 219CZ3 | 300I12 | 60CG | 70.31 |
| 219CZ3 | 300I12 | 62CZ | 86.96 |
| 219CZ3 | 300I12 | 76CB | 91.47 |
| 219CZ3 | 300I12 | 60CB | 57.90 |
| 219CZ3 | 300I12 | 62CE2 | 94.53 |
| 219CZ3 | 300I12 | 219CH2 | 12.86 |
| 219CE3 | 300I12 | 76CG | 81.58 |
| 219CE3 | 300I12 | 62CB | 75.60 |
| 219CE3 | 300I12 | 76NE2 | 90.85 |
| 219CE3 | 300I12 | 62CD2 | 85.27 |
| 219CE3 | 300I12 | 60CG | 90.11 |
| 219CE3 | 300I12 | 62CZ | 73.51 |
| 219CE3 | 300I12 | 76CB | 73.24 |
| 219CE3 | 300I12 | 76ND1 | 97.67 |
| 219CE3 | 300I12 | 60CB | 77.29 |
| 219CE3 | 300I12 | 62CE2 | 84.76 |
| 219CE3 | 300I12 | 219CH2 | 29.04 |
| 76CG | 300I12 | 76NE2 | 29.59 |
| 76CG | 300I12 | 62CZ | 86.56 |
| 76CG | 300I12 | 76CB | 17.98 |
| 76CG | 300I12 | 76ND1 | 16.26 |
| 76CG | 300I12 | 76CE1 | 27.22 |
| 76CG | 300I12 | 62CE2 | 99.91 |
| 62CB | 300I12 | 62CD2 | 32.73 |
| 62CB | 300I12 | 60CG | 73.49 |
| 62CB | 300I12 | 62CZ | 57.73 |
| 62CB | 300I12 | 56CB | 47.23 |
| 62CB | 300I12 | 60CB | 58.21 |
| 62CB | 300I12 | 62CE2 | 48.19 |
| 62CB | 300I12 | 219CH2 | 58.26 |
| 76NE2 | 300I12 | 76CB | 47.29 |
| 76NE2 | 300I12 | 76ND1 | 26.61 |
| 76NE2 | 300I12 | 76CE1 | 15.46 |
| 62CD2 | 300I12 | 62CZ | 30.15 |
| 62CD2 | 300I12 | 56CB | 48.14 |
| 62CD2 | 300I12 | 76CB | 98.98 |
| 62CD2 | 300I12 | 60CB | 90.70 |
| 62CD2 | 300I12 | 62CE2 | 16.30 |
| 62CD2 | 300I12 | 219CH2 | 81.38 |
| 60CG | 300I12 | 56CB | 75.63 |
| 60CG | 300I12 | 60CB | 18.10 |
| 60CG | 300I12 | 219CH2 | 63.77 |
| 62CZ | 300I12 | 56CB | 75.64 |
| 62CZ | 300I12 | 76CB | 69.07 |
| 62CZ | 300I12 | 76ND1 | 93.38 |
| 62CZ | 300I12 | 62CE2 | 16.25 |
| 62CZ | 300I12 | 219CH2 | 84.48 |
| 56CB | 300I12 | 60CB | 73.12 |
| 56CB | 300I12 | 62CE2 | 59.40 |
| 76CB | 300I12 | 76ND1 | 30.65 |
| 76CB | 300I12 | 76CE1 | 44.50 |
| 76CB | 300I12 | 62CE2 | 83.25 |
| 76ND1 | 300I12 | 76CE1 | 15.82 |
| 60CB | 300I12 | 219CH2 | 48.92 |
| 62CE2 | 300I12 | 219CH2 | 88.84 |
| 104OD2 | 253ZN | 168NE2 | 94.40 |
| 104OD2 | 253ZN | 233OE2 | 74.39 |
| 104OD2 | 253ZN | 168CE1 | 73.00 |
| 104OD2 | 253ZN | 104CG | 14.18 |
| 104OD2 | 253ZN | 104OD1 | 34.22 |
| 104OD2 | 253ZN | 233CD | 71.69 |
| 104OD2 | 253ZN | 233OE1 | 72.33 |
| 104OD2 | 253ZN | 168ND1 | 83.25 |
| 104OD2 | 253ZN | 104CB | 4.87 |
| 104OD2 | 253ZN | 233CG | 70.68 |
| 104OD2 | 253ZN | 174CD1 | 57.15 |
| 104OD2 | 253ZN | 93OD2 | 90.69 |
| 168NE2 | 253ZN | 202OE1 | 88.08 |
| 168NE2 | 253ZN | 168CE1 | 21.44 |
| 168NE2 | 253ZN | 168CD2 | 21.13 |
| 168NE2 | 253ZN | 168ND1 | 11.72 |
| 168NE2 | 253ZN | 168CG | 10.72 |
| 168NE2 | 253ZN | 104CB | 96.50 |
| 168NE2 | 253ZN | 175CE1 | 46.01 |
| 168NE2 | 253ZN | 174CD1 | 72.14 |
| 233OE2 | 253ZN | 202OE1 | 76.50 |
| 233OE2 | 253ZN | 104CG | 66.35 |
| 233OE2 | 253ZN | 202CD | 59.18 |
| 233OE2 | 253ZN | 104OD1 | 61.23 |
| 233OE2 | 253ZN | 233CD | 10.43 |
| 233OE2 | 253ZN | 202OE2 | 56.28 |
| 233OE2 | 253ZN | 233OE1 | 27.78 |
| 233OE2 | 253ZN | 104CB | 69.71 |
| 233OE2 | 253ZN | 202CG | 52.63 |
| 233OE2 | 253ZN | 233CG | 4.26 |
| 233OE2 | 253ZN | 93OD2 | 59.04 |
| 202OE1 | 253ZN | 168CD2 | 67.86 |
| 202OE1 | 253ZN | 202CD | 19.49 |
| 202OE1 | 253ZN | 233CD | 83.25 |
| 202OE1 | 253ZN | 202OE2 | 36.04 |
| 202OE1 | 253ZN | 233OE1 | 91.70 |
| 202OE1 | 253ZN | 168ND1 | 94.81 |
| 202OE1 | 253ZN | 168CG | 77.48 |
| 202OE1 | 253ZN | 202CG | 24.41 |
| 202OE1 | 253ZN | 233CG | 79.13 |
| 202OE1 | 253ZN | 93OD2 | 93.50 |
| 168CE1 | 253ZN | 104CG | 87.09 |
| 168CE1 | 253ZN | 168CD2 | 42.10 |
| 168CE1 | 253ZN | 168ND1 | 11.37 |
| 168CE1 | 253ZN | 168CG | 30.24 |
| 168CE1 | 253ZN | 104CB | 75.27 |
| 168CE1 | 253ZN | 175CE1 | 55.05 |
| 168CE1 | 253ZN | 174CD1 | 59.29 |
| 104CG | 253ZN | 104OD1 | 20.26 |
| 104CG | 253ZN | 233CD | 61.66 |
| 104CG | 253ZN | 233OE1 | 59.57 |
| 104CG | 253ZN | 168ND1 | 97.42 |
| 104CG | 253ZN | 104CB | 13.28 |
| 104CG | 253ZN | 233CG | 63.20 |
| 104CG | 253ZN | 174CD1 | 62.71 |
| 104CG | 253ZN | 93OD2 | 76.54 |
| 168CD2 | 253ZN | 202CD | 86.80 |
| 168CD2 | 253ZN | 168ND1 | 31.14 |
| 168CD2 | 253ZN | 168CG | 12.27 |
| 168CD2 | 253ZN | 202CG | 84.09 |
| 168CD2 | 253ZN | 175CE1 | 49.79 |
| 168CD2 | 253ZN | 174CD1 | 90.11 |
| 202CD | 253ZN | 233CD | 64.75 |
| 202CD | 253ZN | 202OE2 | 19.78 |
| 202CD | 253ZN | 233OE1 | 72.22 |
| 202CD | 253ZN | 168CG | 95.56 |
| 202CD | 253ZN | 202CG | 14.32 |
| 202CD | 253ZN | 233CG | 62.36 |
| 202CD | 253ZN | 93OD2 | 76.87 |
| 104OD1 | 253ZN | 233CD | 53.39 |
| 104OD1 | 253ZN | 233OE1 | 45.35 |
| 104OD1 | 253ZN | 104CB | 33.47 |
| 104OD1 | 253ZN | 233CG | 59.41 |
| 104OD1 | 253ZN | 174CD1 | 69.10 |
| 104OD1 | 253ZN | 93OD2 | 56.56 |
| 233CD | 253ZN | 202OE2 | 58.09 |
| 233CD | 253ZN | 233OE1 | 17.51 |
| 233CD | 253ZN | 104CB | 67.35 |
| 233CD | 253ZN | 202CG | 60.35 |
| 233CD | 253ZN | 233CG | 12.10 |
| 233CD | 253ZN | 93OD2 | 49.89 |
| 202OE2 | 253ZN | 233OE1 | 59.91 |
| 202OE2 | 253ZN | 202CG | 31.41 |
| 202OE2 | 253ZN | 233CG | 60.31 |
| 202OE2 | 253ZN | 93OD2 | 57.74 |
| 233OE1 | 253ZN | 104CB | 68.93 |
| 233OE1 | 253ZN | 202CG | 71.73 |
| 233OE1 | 253ZN | 233CG | 29.55 |
| 233OE1 | 253ZN | 93OD2 | 33.55 |
| 168ND1 | 253ZN | 168CG | 19.04 |
| 168ND1 | 253ZN | 104CB | 85.10 |
| 168ND1 | 253ZN | 175CE1 | 53.83 |
| 168ND1 | 253ZN | 174CD1 | 68.77 |
| 168CG | 253ZN | 202CG | 90.55 |
| 168CG | 253ZN | 175CE1 | 51.36 |
| 168CG | 253ZN | 174CD1 | 82.76 |
| 104CB | 253ZN | 233CG | 65.96 |
| 104CB | 253ZN | 174CD1 | 62.01 |
| 104CB | 253ZN | 93OD2 | 89.31 |
| 202CG | 253ZN | 233CG | 54.97 |
| 202CG | 253ZN | 93OD2 | 84.33 |
| 175CE1 | 253ZN | 174CD1 | 53.82 |
| 233CG | 253ZN | 93OD2 | 61.82 |
| 93OD1 | 254ZN | 93OD2 | 62.92 |
| 93OD1 | 254ZN | 93CG | 31.47 |
| 93OD1 | 254ZN | 93CB | 28.71 |
| 93OD1 | 254ZN | 93CA | 20.63 |
| 93OD1 | 254ZN | 104C | 96.27 |
| 93OD1 | 254ZN | 104CA | 97.12 |
| 93OD1 | 254ZN | 95CB | 69.85 |
| 93OD1 | 254ZN | 93C | 6.97 |
| 233OE1 | 254ZN | 104OD1 | 94.44 |
| 233OE1 | 254ZN | 93OD2 | 80.38 |
| 233OE1 | 254ZN | 93CG | 93.64 |
| 233OE1 | 254ZN | 233CD | 24.22 |
| 233OE1 | 254ZN | 233OE2 | 48.56 |
| 233OE1 | 254ZN | 104CG | 92.12 |
| 233OE1 | 254ZN | 104OD2 | 94.04 |
| 233OE1 | 254ZN | 93CB | 94.96 |
| 233OE1 | 254ZN | 233CG | 19.36 |
| 233OE1 | 254ZN | 104CB | 88.80 |
| 233OE1 | 254ZN | 202OE2 | 61.38 |
| 233OE1 | 254ZN | 93CA | 87.96 |
| 233OE1 | 254ZN | 104C | 66.56 |
| 233OE1 | 254ZN | 104CA | 84.36 |
| 233OE1 | 254ZN | 93C | 99.74 |
| 233OE1 | 254ZN | 233CB | 3.02 |
| 104OD1 | 254ZN | 233CD | 82.02 |
| 104OD1 | 254ZN | 233OE2 | 77.86 |
| 104OD1 | 254ZN | 104CG | 19.27 |
| 104OD1 | 254ZN | 104OD2 | 41.51 |
| 104OD1 | 254ZN | 233CG | 79.14 |
| 104OD1 | 254ZN | 104CB | 11.46 |
| 104OD1 | 254ZN | 104C | 31.12 |
| 104OD1 | 254ZN | 104CA | 13.12 |
| 104OD1 | 254ZN | 95CB | 44.32 |
| 104OD1 | 254ZN | 93C | 99.81 |
| 104OD1 | 254ZN | 233CB | 93.51 |
| 93OD2 | 254ZN | 93CG | 31.45 |
| 93OD2 | 254ZN | 233CD | 98.64 |
| 93OD2 | 254ZN | 93CB | 34.22 |
| 93OD2 | 254ZN | 233CG | 98.59 |
| 93OD2 | 254ZN | 202OE2 | 72.78 |
| 93OD2 | 254ZN | 93CA | 48.01 |
| 93OD2 | 254ZN | 93C | 64.27 |
| 93OD2 | 254ZN | 233CB | 82.14 |
| 93CG | 254ZN | 93CB | 2.77 |
| 93CG | 254ZN | 93CA | 19.90 |
| 93CG | 254ZN | 95CB | 99.73 |
| 93CG | 254ZN | 93C | 33.28 |
| 93CG | 254ZN | 233CB | 96.36 |
| 233CD | 254ZN | 233OE2 | 25.03 |
| 233CD | 254ZN | 104CG | 73.74 |
| 233CD | 254ZN | 104OD2 | 70.98 |
| 233CD | 254ZN | 233CG | 9.27 |
| 233CD | 254ZN | 104CB | 73.33 |
| 233CD | 254ZN | 202OE2 | 53.06 |
| 233CD | 254ZN | 104C | 61.95 |
| 233CD | 254ZN | 104CA | 75.81 |
| 233CD | 254ZN | 233CB | 21.28 |
| 233OE2 | 254ZN | 104CG | 63.04 |
| 233OE2 | 254ZN | 104OD2 | 52.10 |
| 233OE2 | 254ZN | 233CG | 33.32 |
| 233OE2 | 254ZN | 104CB | 66.81 |
| 233OE2 | 254ZN | 202OE2 | 44.20 |
| 233OE2 | 254ZN | 104C | 70.21 |
| 233OE2 | 254ZN | 104CA | 77.23 |
| 233OE2 | 254ZN | 233CB | 45.54 |
| 104CG | 254ZN | 104OD2 | 22.51 |
| 104CG | 254ZN | 233CG | 73.79 |
| 104CG | 254ZN | 104CB | 9.75 |
| 104CG | 254ZN | 104C | 41.30 |
| 104CG | 254ZN | 104CA | 28.60 |
| 104CG | 254ZN | 95CB | 57.30 |
| 104CG | 254ZN | 233CB | 90.30 |
| 104OD2 | 254ZN | 233CG | 74.72 |
| 104OD2 | 254ZN | 104CB | 32.05 |
| 104OD2 | 254ZN | 202OE2 | 83.62 |
| 104OD2 | 254ZN | 104C | 60.90 |
| 104OD2 | 254ZN | 104CA | 50.89 |
| 104OD2 | 254ZN | 95CB | 71.64 |
| 104OD2 | 254ZN | 233CB | 91.44 |
| 93CB | 254ZN | 93CA | 18.04 |
| 93CB | 254ZN | 95CB | 97.05 |
| 93CB | 254ZN | 93C | 30.63 |
| 93CB | 254ZN | 233CB | 97.73 |
| 233CG | 254ZN | 104CB | 71.76 |
| 233CG | 254ZN | 202OE2 | 61.28 |
| 233CG | 254ZN | 104C | 55.74 |
| 233CG | 254ZN | 104CA | 71.25 |
| 233CG | 254ZN | 233CB | 16.95 |
| 104CB | 254ZN | 104C | 32.31 |
| 104CB | 254ZN | 104CA | 18.89 |
| 104CB | 254ZN | 95CB | 54.38 |
| 104CB | 254ZN | 233CB | 87.40 |
| 202OE2 | 254ZN | 233CB | 59.45 |
| 93CA | 254ZN | 104C | 98.76 |
| 93CA | 254ZN | 95CB | 89.31 |
| 93CA | 254ZN | 93C | 18.16 |
| 93CA | 254ZN | 233CB | 90.97 |
| 104C | 254ZN | 104CA | 18.64 |
| 104C | 254ZN | 95CB | 65.97 |
| 104C | 254ZN | 93C | 90.76 |
| 104C | 254ZN | 233CB | 66.41 |
| 104CA | 254ZN | 95CB | 50.13 |
| 104CA | 254ZN | 93C | 93.27 |
| 104CA | 254ZN | 233CB | 83.88 |
| 95CB | 254ZN | 93C | 71.15 |

TABLE VII
Provides bond angles between interresidue atoms that
are within 5 Ångstroms apart in an active site of an
S. aureus methionine aminopeptidase for an inhibitor
complex with 5-benzofuran-2-yl-1-H-[1,2,3]triazole.

| Atom 1 | Atom 2 | Atom 3 | Angle |
|---|---|---|---|
| 76CE1 | 300C1 | 76NE2 | 19.14 |
| 76CE1 | 300C1 | 76ND1 | 15.95 |
| 76CE1 | 300C1 | 76CD2 | 26.06 |
| 76CE1 | 300C1 | 175NE2 | 98.82 |
| 76CE1 | 300C1 | 67SG | 74.42 |
| 76NE2 | 300C1 | 76ND1 | 28.72 |
| 76NE2 | 300C1 | 76CD2 | 14.53 |
| 76NE2 | 300C1 | 175NE2 | 91.86 |
| 76NE2 | 300C1 | 67SG | 81.18 |
| 174CD2 | 300C1 | 175CE1 | 52.87 |
| 174CD2 | 300C1 | 175NE2 | 67.72 |
| 174CD2 | 300C1 | 174CG | 15.79 |
| 175CE1 | 300C1 | 175NE2 | 14.91 |
| 175CE1 | 300C1 | 174CG | 42.43 |
| 76ND1 | 300C1 | 76CD2 | 26.83 |
| 76ND1 | 300C1 | 67SG | 58.47 |
| 76CD2 | 300C1 | 67SG | 69.24 |
| 175NE2 | 300C1 | 174CG | 57.13 |
| 174CD2 | 300C2 | 175CE1 | 52.02 |
| 174CD2 | 300C2 | 174CG | 18.44 |
| 174CD2 | 300C2 | 104OD2 | 80.51 |
| 174CD2 | 300C2 | 174CD1 | 31.45 |
| 174CD2 | 300C2 | 95OG | 55.04 |
| 174CD2 | 300C2 | 104OD1 | 88.85 |
| 76CE1 | 300C2 | 93OD2 | 87.27 |
| 76CE1 | 300C2 | 93CG | 95.03 |
| 76CE1 | 300C2 | 76NE2 | 14.74 |
| 175CE1 | 300C2 | 174CG | 43.86 |
| 175CE1 | 300C2 | 104OD2 | 77.13 |
| 175CE1 | 300C2 | 174CD1 | 57.79 |
| 175CE1 | 300C2 | 76NE2 | 91.07 |
| 93OD2 | 300C2 | 93OD1 | 27.20 |
| 93OD2 | 300C2 | 104OD2 | 70.80 |
| 93OD2 | 300C2 | 93CG | 14.93 |
| 93OD2 | 300C2 | 95OG | 87.95 |
| 93OD2 | 300C2 | 104OD1 | 53.47 |
| 93OD1 | 300C2 | 104OD2 | 63.87 |
| 93OD1 | 300C2 | 174CD1 | 91.49 |
| 93OD1 | 300C2 | 93CG | 14.94 |
| 93OD1 | 300C2 | 95OG | 60.76 |
| 93OD1 | 300C2 | 104OD1 | 38.83 |
| 174CG | 300C2 | 104OD2 | 63.86 |
| 174CG | 300C2 | 174CD1 | 18.41 |
| 174CG | 300C2 | 95OG | 58.94 |
| 174CG | 300C2 | 104OD1 | 77.06 |
| 104OD2 | 300C2 | 174CD1 | 49.42 |
| 104OD2 | 300C2 | 93CG | 72.72 |
| 104OD2 | 300C2 | 95OG | 69.61 |
| 104OD2 | 300C2 | 104OD1 | 26.43 |
| 174CD1 | 300C2 | 95OG | 47.37 |
| 174CD1 | 300C2 | 104OD1 | 59.00 |
| 93CG | 300C2 | 95OG | 74.56 |
| 93CG | 300C2 | 104OD1 | 50.31 |
| 95OG | 300C2 | 104OD1 | 54.84 |
| 76CE1 | 300C3 | 67SG | 88.64 |
| 76CE1 | 300C3 | 76NE2 | 19.56 |
| 76CE1 | 300C3 | 76ND1 | 18.60 |
| 76CE1 | 300C3 | 76CD2 | 30.32 |
| 76CE1 | 300C3 | 76CG | 29.53 |
| 67SG | 300C3 | 76NE2 | 97.91 |
| 67SG | 300C3 | 76ND1 | 70.36 |
| 67SG | 300C3 | 76CD2 | 84.94 |
| 67SG | 300C3 | 76CG | 68.93 |
| 67SG | 300C3 | 56CG | 63.16 |
| 76NE2 | 300C3 | 76ND1 | 30.70 |
| 76NE2 | 300C3 | 76CD2 | 18.26 |
| 76NE2 | 300C3 | 76CG | 29.44 |
| 76ND1 | 300C3 | 76CD2 | 29.74 |
| 76ND1 | 300C3 | 76CG | 17.88 |
| 76CD2 | 300C3 | 76CG | 17.62 |
| 174CD2 | 300C3 | 56CG | 71.10 |
| 175CE1 | 300O4 | 175NE2 | 19.62 |
| 175CE1 | 300O4 | 174CD2 | 58.51 |
| 175CE1 | 300O4 | 175ND1 | 9.44 |
| 175CE1 | 300O4 | 174CG | 44.17 |
| 76NE2 | 300O4 | 76CE1 | 20.02 |
| 76NE2 | 300O4 | 76CD2 | 13.84 |
| 76NE2 | 300O4 | 76ND1 | 25.39 |
| 76CE1 | 300O4 | 76CD2 | 27.89 |
| 76CE1 | 300O4 | 76ND1 | 12.39 |
| 175NE2 | 300O4 | 174CD2 | 78.13 |
| 175NE2 | 300O4 | 175ND1 | 27.17 |
| 175NE2 | 300O4 | 174CG | 63.60 |
| 174CD2 | 300O4 | 175ND1 | 51.81 |
| 174CD2 | 300O4 | 174CG | 16.61 |
| 76CD2 | 300O4 | 76ND1 | 26.70 |
| 175ND1 | 300O4 | 174CG | 39.13 |
| 93OD1 | 300C5 | 93CG | 20.13 |
| 93OD1 | 300C5 | 93OD2 | 36.26 |
| 93OD1 | 300C5 | 104OD1 | 50.64 |
| 93OD1 | 300C5 | 95OG | 79.27 |
| 93OD1 | 300C5 | 104OD2 | 78.56 |
| 93OD1 | 300C5 | 104CG | 65.61 |
| 93OD1 | 300C5 | 93CB | 29.58 |
| 93OD1 | 300C5 | 95CB | 69.09 |
| 93OD1 | 300C5 | 233OE1 | 43.84 |
| 93OD1 | 300C5 | 67SG | 88.44 |
| 93CG | 300C5 | 93OD2 | 19.79 |
| 93CG | 300C5 | 104OD1 | 65.68 |
| 93CG | 300C5 | 95OG | 97.90 |
| 93CG | 300C5 | 104OD2 | 89.37 |
| 93CG | 300C5 | 104CG | 79.56 |
| 93CG | 300C5 | 93CB | 15.86 |
| 93CG | 300C5 | 95CB | 89.03 |
| 93CG | 300C5 | 233OE1 | 44.29 |
| 93CG | 300C5 | 67SG | 79.99 |
| 93OD2 | 300C5 | 104OD1 | 68.37 |
| 93OD2 | 300C5 | 104OD2 | 84.21 |
| 93OD2 | 300C5 | 104CG | 79.26 |
| 93OD2 | 300C5 | 93CB | 30.45 |
| 93OD2 | 300C5 | 233OE1 | 35.41 |
| 93OD2 | 300C5 | 67SG | 88.68 |
| 104OD1 | 300C5 | 95OG | 67.01 |
| 104OD1 | 300C5 | 104OD2 | 30.00 |
| 104OD1 | 300C5 | 104CG | 15.11 |
| 104OD1 | 300C5 | 93CB | 79.44 |
| 104OD1 | 300C5 | 95CB | 50.02 |
| 104OD1 | 300C5 | 174CD2 | 93.57 |
| 104OD1 | 300C5 | 233OE1 | 38.72 |
| 104OD1 | 300C5 | 174CD1 | 63.77 |
| 95OG | 300C5 | 104OD2 | 79.50 |
| 95OG | 300C5 | 104CG | 68.75 |
| 95OG | 300C5 | 93CB | 97.18 |
| 95OG | 300C5 | 95CB | 16.99 |
| 95OG | 300C5 | 174CD2 | 56.35 |
| 95OG | 300C5 | 174CD1 | 49.70 |
| 95OG | 300C5 | 67SG | 92.59 |
| 104OD2 | 300C5 | 104CG | 16.02 |
| 104OD2 | 300C5 | 95CB | 64.77 |
| 104OD2 | 300C5 | 174CD2 | 78.21 |
| 104OD2 | 300C5 | 233OE1 | 48.81 |
| 104OD2 | 300C5 | 174CD1 | 50.15 |
| 104CG | 300C5 | 93CB | 93.97 |
| 104CG | 300C5 | 95CB | 52.56 |
| 104CG | 300C5 | 174CD2 | 82.43 |
| 104CG | 300C5 | 233OE1 | 45.66 |
| 104CG | 300C5 | 174CD1 | 52.45 |
| 93CB | 300C5 | 95CB | 92.58 |
| 93CB | 300C5 | 233OE1 | 59.99 |
| 93CB | 300C5 | 67SG | 64.15 |
| 95CB | 300C5 | 174CD2 | 63.84 |
| 95CB | 300C5 | 233OE1 | 86.55 |
| 95CB | 300C5 | 174CD1 | 47.31 |
| 174CD2 | 300C5 | 174CD1 | 30.04 |
| 233OE1 | 300C5 | 174CD1 | 97.53 |
| 168NE2 | 300N6 | 175CE1 | 60.66 |
| 168NE2 | 300N6 | 104OD2 | 50.92 |
| 168NE2 | 300N6 | 175NE2 | 56.93 |
| 168NE2 | 300N6 | 174CD2 | 94.08 |
| 168NE2 | 300N6 | 168CE1 | 15.28 |
| 168NE2 | 300N6 | 174CG | 74.77 |
| 168NE2 | 300N6 | 174CD1 | 72.46 |
| 168NE2 | 300N6 | 104OD1 | 79.88 |
| 168NE2 | 300N6 | 202OE1 | 48.07 |
| 168NE2 | 300N6 | 104CG | 65.13 |
| 168NE2 | 300N6 | 168CD2 | 14.11 |
| 168NE2 | 300N6 | 233OE2 | 56.29 |
| 168NE2 | 300N6 | 175ND1 | 57.85 |
| 168NE2 | 300N6 | 202OE2 | 70.53 |
| 175CE1 | 300N6 | 104OD2 | 98.09 |
| 175CE1 | 300N6 | 175NE2 | 17.84 |
| 175CE1 | 300N6 | 174CD2 | 54.88 |
| 175CE1 | 300N6 | 168CE1 | 58.33 |
| 175CE1 | 300N6 | 174CG | 48.25 |
| 175CE1 | 300N6 | 174CD1 | 64.93 |
| 175CE1 | 300N6 | 202OE1 | 98.82 |
| 175CE1 | 300N6 | 168CD2 | 56.36 |
| 175CE1 | 300N6 | 175ND1 | 9.41 |
| 104OD2 | 300N6 | 174CD2 | 89.62 |
| 104OD2 | 300N6 | 168CE1 | 43.45 |
| 104OD2 | 300N6 | 174CG | 73.82 |
| 104OD2 | 300N6 | 174CD1 | 56.73 |
| 104OD2 | 300N6 | 104OD1 | 29.29 |
| 104OD2 | 300N6 | 202OE1 | 65.42 |
| 104OD2 | 300N6 | 104CG | 14.26 |
| 104OD2 | 300N6 | 168CD2 | 64.32 |
| 104OD2 | 300N6 | 93OD2 | 77.65 |
| 104OD2 | 300N6 | 233OE2 | 35.43 |
| 104OD2 | 300N6 | 175ND1 | 90.27 |
| 104OD2 | 300N6 | 202OE2 | 66.17 |
| 175NE2 | 300N6 | 174CD2 | 72.37 |
| 175NE2 | 300N6 | 168CE1 | 60.10 |
| 175NE2 | 300N6 | 174CG | 65.80 |
| 175NE2 | 300N6 | 174CD1 | 81.37 |
| 175NE2 | 300N6 | 202OE1 | 85.55 |
| 175NE2 | 300N6 | 168CD2 | 48.21 |
| 175NE2 | 300N6 | 175ND1 | 25.94 |
| 175NE2 | 300N6 | 76CE1 | 93.02 |
| 174CD2 | 300N6 | 168CE1 | 81.04 |
| 174CD2 | 300N6 | 174CG | 19.90 |
| 174CD2 | 300N6 | 174CD1 | 32.93 |
| 174CD2 | 300N6 | 104OD1 | 91.82 |
| 174CD2 | 300N6 | 104CG | 86.97 |
| 174CD2 | 300N6 | 168CD2 | 99.59 |
| 174CD2 | 300N6 | 175ND1 | 49.15 |
| 168CE1 | 300N6 | 174CG | 61.25 |
| 168CE1 | 300N6 | 174CD1 | 57.27 |
| 168CE1 | 300N6 | 104OD1 | 72.53 |
| 168CE1 | 300N6 | 202OE1 | 61.10 |
| 168CE1 | 300N6 | 104CG | 56.75 |
| 168CE1 | 300N6 | 168CD2 | 28.01 |
| 168CE1 | 300N6 | 233OE2 | 59.87 |
| 168CE1 | 300N6 | 175ND1 | 52.93 |
| 168CE1 | 300N6 | 202OE2 | 80.57 |
| 174CG | 300N6 | 174CD1 | 19.60 |
| 174CG | 300N6 | 104OD1 | 83.78 |
| 174CG | 300N6 | 104CG | 74.67 |
| 174CG | 300N6 | 168CD2 | 81.82 |
| 174CG | 300N6 | 175ND1 | 39.92 |
| 174CD1 | 300N6 | 104OD1 | 64.19 |
| 174CD1 | 300N6 | 104CG | 55.61 |
| 174CD1 | 300N6 | 168CD2 | 83.26 |
| 174CD1 | 300N6 | 233OE2 | 92.15 |
| 174CD1 | 300N6 | 175ND1 | 55.79 |
| 104OD1 | 300N6 | 202OE1 | 80.00 |
| 104OD1 | 300N6 | 104CG | 15.94 |
| 104OD1 | 300N6 | 168CD2 | 92.72 |
| 104OD1 | 300N6 | 93OD2 | 55.13 |
| 104OD1 | 300N6 | 233OE2 | 42.79 |
| 104OD1 | 300N6 | 202OE2 | 67.63 |
| 202OE1 | 300N6 | 104CG | 75.06 |
| 202OE1 | 300N6 | 168CD2 | 44.66 |
| 202OE1 | 300N6 | 93OD2 | 74.87 |
| 202OE1 | 300N6 | 233OE2 | 37.23 |
| 202OE1 | 300N6 | 76CE1 | 96.91 |
| 202OE1 | 300N6 | 202OE2 | 26.19 |
| 104CG | 300N6 | 168CD2 | 78.58 |
| 104CG | 300N6 | 93OD2 | 69.09 |
| 104CG | 300N6 | 233OE2 | 40.23 |
| 104CG | 300N6 | 175ND1 | 99.04 |
| 104CG | 300N6 | 202OE2 | 69.75 |
| 168CD2 | 300N6 | 233OE2 | 63.34 |
| 168CD2 | 300N6 | 175ND1 | 56.18 |
| 168CD2 | 300N6 | 202OE2 | 70.04 |
| 93OD2 | 300N6 | 233OE2 | 56.91 |
| 93OD2 | 300N6 | 76CE1 | 79.88 |
| 93OD2 | 300N6 | 202OE2 | 48.71 |
| 233OE2 | 300N6 | 202OE2 | 30.74 |
| 76CE1 | 300N6 | 202OE2 | 89.70 |
| 76NE2 | 300C7 | 76CD2 | 21.35 |
| 76NE2 | 300C7 | 76CE1 | 19.73 |
| 76NE2 | 300C7 | 76CG | 30.99 |
| 76NE2 | 300C7 | 76ND1 | 29.72 |
| 76NE2 | 300C7 | 67SG | 91.58 |
| 76CD2 | 300C7 | 76CE1 | 33.21 |
| 76CD2 | 300C7 | 76CG | 18.17 |
| 76CD2 | 300C7 | 76ND1 | 30.49 |
| 76CD2 | 300C7 | 67SG | 82.30 |
| 76CE1 | 300C7 | 76CG | 30.96 |
| 76CE1 | 300C7 | 76ND1 | 17.67 |
| 76CE1 | 300C7 | 67SG | 77.73 |
| 60CG | 300C7 | 60CD | 17.36 |
| 76CG | 300C7 | 76ND1 | 18.51 |
| 76CG | 300C7 | 67SG | 64.49 |
| 76ND1 | 300C7 | 67SG | 62.25 |
| 76NE2 | 300C8 | 76CE1 | 19.24 |
| 76NE2 | 300C8 | 76CD2 | 19.05 |
| 76NE2 | 300C8 | 76ND1 | 24.47 |
| 76NE2 | 300C8 | 76CG | 24.20 |
| 76CE1 | 300C8 | 76CD2 | 32.14 |
| 76CE1 | 300C8 | 76ND1 | 14.15 |
| 76CE1 | 300C8 | 175NE2 | 99.96 |
| 76CE1 | 300C8 | 76CG | 27.00 |
| 76CD2 | 300C8 | 76ND1 | 27.63 |
| 76CD2 | 300C8 | 76CG | 14.59 |
| 60CG | 300C8 | 175CE1 | 95.50 |
| 60CG | 300C8 | 60CD | 19.94 |
| 60CG | 300C8 | 60OE1 | 31.19 |
| 60CG | 300C8 | 174CD2 | 66.15 |
| 175CE1 | 300C8 | 60CD | 82.18 |
| 175CE1 | 300C8 | 60OE1 | 87.00 |
| 175CE1 | 300C8 | 174CD2 | 48.76 |
| 175CE1 | 300C8 | 175NE2 | 16.06 |
| 60CD | 300C8 | 60OE1 | 15.83 |
| 60CD | 300C8 | 174CD2 | 67.06 |
| 60CD | 300C8 | 175NE2 | 89.46 |
| 60OE1 | 300C8 | 174CD2 | 81.55 |
| 60OE1 | 300C8 | 175NE2 | 90.17 |
| 174CD2 | 300C8 | 175NE2 | 64.81 |
| 76ND1 | 300C8 | 76CG | 16.66 |
| 104OD1 | 300N9 | 93OD1 | 67.33 |
| 104OD1 | 300N9 | 93OD2 | 91.27 |
| 104OD1 | 300N9 | 104OD2 | 42.33 |
| 104OD1 | 300N9 | 93CG | 84.17 |
| 104OD1 | 300N9 | 104CG | 21.21 |
| 104OD1 | 300N9 | 233OE1 | 54.45 |
| 104OD1 | 300N9 | 233OE2 | 56.07 |
| 104OD1 | 300N9 | 233CD | 50.71 |
| 104OD1 | 300N9 | 95OG | 73.08 |
| 104OD1 | 300N9 | 95CB | 55.01 |
| 104OD1 | 300N9 | 202OE2 | 87.17 |
| 104OD1 | 300N9 | 93CB | 92.10 |
| 104OD1 | 300N9 | 174CD1 | 73.50 |
| 104OD1 | 300N9 | 168NE2 | 84.53 |
| 104OD1 | 300N9 | 104CB | 21.22 |
| 104OD1 | 300N9 | 202OE1 | 91.43 |
| 93OD1 | 300N9 | 93OD2 | 41.87 |
| 93OD1 | 300N9 | 93CG | 21.87 |
| 93OD1 | 300N9 | 104CG | 88.49 |
| 93OD1 | 300N9 | 233OE1 | 60.03 |
| 93OD1 | 300N9 | 233OE2 | 91.88 |
| 93OD1 | 300N9 | 233CD | 74.45 |
| 93OD1 | 300N9 | 95OG | 79.50 |
| 93OD1 | 300N9 | 95CB | 74.48 |
| 93OD1 | 300N9 | 202OE2 | 92.50 |
| 93OD1 | 300N9 | 93CB | 25.77 |
| 93OD1 | 300N9 | 104CB | 87.91 |
| 93OD2 | 300N9 | 93CG | 21.96 |
| 93OD2 | 300N9 | 233OE1 | 48.11 |
| 93OD2 | 300N9 | 233OE2 | 77.20 |
| 93OD2 | 300N9 | 233CD | 63.02 |
| 93OD2 | 300N9 | 202OE2 | 58.74 |
| 93OD2 | 300N9 | 93CB | 27.95 |
| 93OD2 | 300N9 | 202OE1 | 84.27 |
| 104OD2 | 300N9 | 104CG | 21.90 |
| 104OD2 | 300N9 | 233OE1 | 68.14 |
| 104OD2 | 300N9 | 233OE2 | 43.26 |
| 104OD2 | 300N9 | 233CD | 53.81 |
| 104OD2 | 300N9 | 95OG | 90.32 |
| 104OD2 | 300N9 | 95CB | 75.87 |
| 104OD2 | 300N9 | 202OE2 | 74.95 |
| 104OD2 | 300N9 | 174CD1 | 56.27 |
| 104OD2 | 300N9 | 168NE2 | 42.37 |
| 104OD2 | 300N9 | 104CB | 25.94 |
| 104OD2 | 300N9 | 202OE1 | 62.73 |
| 93CG | 300N9 | 233OE1 | 57.69 |
| 93CG | 300N9 | 233OE2 | 90.42 |
| 93CG | 300N9 | 233CD | 73.65 |
| 93CG | 300N9 | 95OG | 97.71 |
| 93CG | 300N9 | 95CB | 95.53 |
| 93CG | 300N9 | 202OE2 | 79.21 |
| 93CG | 300N9 | 93CB | 10.65 |
| 104CG | 300N9 | 233OE1 | 63.05 |
| 104CG | 300N9 | 233OE2 | 50.44 |
| 104CG | 300N9 | 233CD | 53.11 |
| 104CG | 300N9 | 95OG | 77.28 |
| 104CG | 300N9 | 95CB | 60.27 |
| 104CG | 300N9 | 202OE2 | 84.53 |
| 104CG | 300N9 | 174CD1 | 59.66 |
| 104CG | 300N9 | 168NE2 | 64.22 |
| 104CG | 300N9 | 104CB | 6.81 |
| 104CG | 300N9 | 202OE1 | 79.83 |
| 233OE1 | 300N9 | 233OE2 | 33.02 |
| 233OE1 | 300N9 | 233CD | 15.97 |
| 233OE1 | 300N9 | 202OE2 | 40.30 |
| 233OE1 | 300N9 | 93CB | 68.23 |
| 233OE1 | 300N9 | 168NE2 | 88.11 |
| 233OE1 | 300N9 | 104CB | 68.47 |
| 233OE1 | 300N9 | 202OE1 | 60.24 |
| 233OE2 | 300N9 | 233CD | 17.51 |
| 233OE2 | 300N9 | 202OE2 | 34.11 |
| 233OE2 | 300N9 | 174CD1 | 98.97 |
| 233OE2 | 300N9 | 168NE2 | 55.17 |
| 233OE2 | 300N9 | 104CB | 57.25 |
| 233OE2 | 300N9 | 202OE1 | 36.75 |
| 233CD | 300N9 | 202OE2 | 36.85 |
| 233CD | 300N9 | 93CB | 84.19 |
| 233CD | 300N9 | 168NE2 | 72.63 |
| 233CD | 300N9 | 104CB | 59.40 |
| 233CD | 300N9 | 202OE1 | 49.92 |
| 95OG | 300N9 | 95CB | 18.09 |
| 95OG | 300N9 | 93CB | 93.26 |
| 95OG | 300N9 | 174CD1 | 50.01 |
| 95OG | 300N9 | 104CB | 70.50 |
| 95CB | 300N9 | 93CB | 94.31 |
| 95CB | 300N9 | 174CD1 | 49.20 |
| 95CB | 300N9 | 104CB | 53.61 |
| 202OE2 | 300N9 | 93CB | 86.68 |
| 202OE2 | 300N9 | 168NE2 | 66.02 |
| 202OE2 | 300N9 | 104CB | 91.34 |
| 202OE2 | 300N9 | 202OE1 | 26.02 |
| 174CD1 | 300N9 | 168NE2 | 61.74 |
| 174CD1 | 300N9 | 104CB | 55.02 |
| 168NE2 | 300N9 | 104CB | 67.39 |
| 168NE2 | 300N9 | 202OE1 | 41.02 |
| 104CB | 300N9 | 202OE1 | 86.09 |
| 104OD2 | 300N10 | 104OD1 | 41.92 |
| 104OD2 | 300N10 | 104CG | 20.72 |
| 104OD2 | 300N10 | 168NE2 | 60.90 |
| 104OD2 | 300N10 | 233OE2 | 49.96 |
| 104OD2 | 300N10 | 233OE1 | 68.40 |
| 104OD2 | 300N10 | 202OE1 | 82.23 |
| 104OD2 | 300N10 | 233CD | 56.26 |
| 104OD2 | 300N10 | 93OD1 | 90.74 |
| 104OD2 | 300N10 | 168CE1 | 48.25 |
| 104OD2 | 300N10 | 202OE2 | 87.71 |
| 104OD2 | 300N10 | 174CD1 | 61.83 |
| 104OD2 | 300N10 | 202CD | 81.05 |
| 104OD2 | 300N10 | 168CD2 | 70.97 |
| 104OD2 | 300N10 | 175CE1 | 97.18 |
| 104OD2 | 300N10 | 104CB | 18.37 |
| 104OD2 | 300N10 | 174CG | 76.46 |
| 104OD2 | 300N10 | 174CD2 | 91.88 |
| 104OD1 | 300N10 | 104CG | 21.75 |
| 104OD1 | 300N10 | 233OE2 | 59.10 |
| 104OD1 | 300N10 | 233OE1 | 48.81 |
| 104OD1 | 300N10 | 93OD2 | 71.62 |
| 104OD1 | 300N10 | 233CD | 50.39 |
| 104OD1 | 300N10 | 93OD1 | 49.29 |
| 104OD1 | 300N10 | 168CE1 | 89.80 |
| 104OD1 | 300N10 | 202OE2 | 89.52 |
| 104OD1 | 300N10 | 93CG | 62.88 |
| 104OD1 | 300N10 | 174CD1 | 74.41 |
| 104OD1 | 300N10 | 202CD | 94.20 |
| 104OD1 | 300N10 | 104CB | 27.15 |
| 104OD1 | 300N10 | 174CG | 92.46 |
| 104OD1 | 300N10 | 174CD2 | 98.46 |
| 104CG | 300N10 | 168NE2 | 81.55 |
| 104CG | 300N10 | 233OE2 | 54.99 |
| 104CG | 300N10 | 233OE1 | 59.95 |
| 104CG | 300N10 | 93OD2 | 91.28 |
| 104CG | 300N10 | 202OE1 | 96.30 |
| 104CG | 300N10 | 233CD | 53.81 |
| 104CG | 300N10 | 93OD1 | 71.03 |
| 104CG | 300N10 | 168CE1 | 68.08 |
| 104CG | 300N10 | 202OE2 | 91.96 |
| 104CG | 300N10 | 93CG | 84.23 |
| 104CG | 300N10 | 174CD1 | 63.17 |
| 104CG | 300N10 | 202CD | 90.60 |
| 104CG | 300N10 | 168CD2 | 91.69 |
| 104CG | 300N10 | 104CB | 6.87 |
| 104CG | 300N10 | 174CG | 80.73 |
| 104CG | 300N10 | 174CD2 | 92.02 |
| 168NE2 | 300N10 | 233OE2 | 70.99 |
| 168NE2 | 300N10 | 202OE1 | 53.66 |
| 168NE2 | 300N10 | 233CD | 87.94 |
| 168NE2 | 300N10 | 168CE1 | 17.13 |
| 168NE2 | 300N10 | 202OE2 | 82.91 |
| 168NE2 | 300N10 | 174CD1 | 74.30 |
| 168NE2 | 300N10 | 202CD | 66.81 |
| 168NE2 | 300N10 | 168CD2 | 11.35 |
| 168NE2 | 300N10 | 175CE1 | 52.09 |
| 168NE2 | 300N10 | 104CB | 77.85 |
| 168NE2 | 300N10 | 174CG | 70.92 |
| 168NE2 | 300N10 | 174CD2 | 86.03 |
| 233OE2 | 300N10 | 233OE1 | 34.46 |
| 233OE2 | 300N10 | 93OD2 | 73.52 |
| 233OE2 | 300N10 | 202OE1 | 45.76 |
| 233OE2 | 300N10 | 233CD | 17.07 |
| 233OE2 | 300N10 | 93OD1 | 81.56 |
| 233OE2 | 300N10 | 168CE1 | 72.64 |
| 233OE2 | 300N10 | 202OE2 | 38.11 |
| 233OE2 | 300N10 | 93CG | 81.06 |
| 233OE2 | 300N10 | 202CD | 36.07 |
| 233OE2 | 300N10 | 168CD2 | 72.78 |
| 233OE2 | 300N10 | 104CB | 59.52 |
| 233OE1 | 300N10 | 93OD2 | 41.19 |
| 233OE1 | 300N10 | 202OE1 | 69.28 |
| 233OE1 | 300N10 | 233CD | 17.60 |
| 233OE1 | 300N10 | 93OD1 | 48.36 |
| 233OE1 | 300N10 | 202OE2 | 43.49 |
| 233OE1 | 300N10 | 93CG | 46.65 |
| 233OE1 | 300N10 | 202CD | 54.15 |
| 233OE1 | 300N10 | 104CB | 66.72 |
| 93OD2 | 300N10 | 202OE1 | 89.54 |
| 93OD2 | 300N10 | 233CD | 58.07 |
| 93OD2 | 300N10 | 93OD1 | 31.19 |
| 93OD2 | 300N10 | 202OE2 | 58.88 |
| 93OD2 | 300N10 | 93CG | 15.95 |
| 93OD2 | 300N10 | 202CD | 74.99 |
| 93OD2 | 300N10 | 104CB | 97.79 |
| 202OE1 | 300N10 | 233CD | 57.83 |
| 202OE1 | 300N10 | 168CE1 | 67.58 |
| 202OE1 | 300N10 | 202OE2 | 31.00 |
| 202OE1 | 300N10 | 202CD | 15.72 |
| 202OE1 | 300N10 | 168CD2 | 46.72 |
| 202OE1 | 300N10 | 175CE1 | 92.31 |
| 202OE1 | 300N10 | 104CB | 98.44 |
| 233CD | 300N10 | 93OD1 | 64.63 |
| 233CD | 300N10 | 168CE1 | 88.20 |
| 233CD | 300N10 | 202OE2 | 39.53 |
| 233CD | 300N10 | 93CG | 64.23 |
| 233CD | 300N10 | 202CD | 44.64 |
| 233CD | 300N10 | 168CD2 | 89.80 |
| 233CD | 300N10 | 104CB | 59.85 |
| 93OD1 | 300N10 | 202OE2 | 84.08 |
| 93OD1 | 300N10 | 93CG | 16.30 |
| 93OD1 | 300N10 | 202CD | 98.85 |
| 93OD1 | 300N10 | 104CB | 76.16 |
| 168CE1 | 300N10 | 202OE2 | 93.99 |
| 168CE1 | 300N10 | 174CD1 | 59.33 |
| 168CE1 | 300N10 | 202CD | 78.57 |
| 168CE1 | 300N10 | 168CD2 | 28.38 |
| 168CE1 | 300N10 | 175CE1 | 53.85 |
| 168CE1 | 300N10 | 104CB | 63.39 |
| 168CE1 | 300N10 | 174CG | 59.66 |
| 168CE1 | 300N10 | 174CD2 | 76.62 |
| 202OE2 | 300N10 | 93CG | 73.54 |
| 202OE2 | 300N10 | 202CD | 16.15 |
| 202OE2 | 300N10 | 168CD2 | 77.36 |
| 202OE2 | 300N10 | 104CB | 97.16 |
| 93CG | 300N10 | 202CD | 89.39 |
| 93CG | 300N10 | 104CB | 90.02 |
| 174CD1 | 300N10 | 168CD2 | 83.41 |
| 174CD1 | 300N10 | 175CE1 | 58.32 |
| 174CD1 | 300N10 | 104CB | 56.74 |
| 174CD1 | 300N10 | 174CG | 18.11 |
| 174CD1 | 300N10 | 174CD2 | 30.15 |
| 202CD | 300N10 | 168CD2 | 61.30 |
| 202CD | 300N10 | 104CB | 94.35 |
| 168CD2 | 300N10 | 175CE1 | 51.16 |
| 168CD2 | 300N10 | 104CB | 88.46 |
| 168CD2 | 300N10 | 174CG | 77.49 |
| 168CD2 | 300N10 | 174CD2 | 90.76 |
| 175CE1 | 300N10 | 174CG | 41.85 |
| 175CE1 | 300N10 | 174CD2 | 45.70 |
| 104CB | 300N10 | 174CG | 74.10 |
| 104CB | 300N10 | 174CD2 | 86.01 |
| 174CG | 300N10 | 174CD2 | 17.78 |
| 76CD2 | 300C11 | 76NE2 | 20.21 |
| 76CD2 | 300C11 | 219CZ3 | 97.90 |
| 76CD2 | 300C11 | 76CG | 17.87 |
| 76CD2 | 300C11 | 219CE3 | 83.02 |
| 76CD2 | 300C11 | 62CE1 | 86.24 |
| 76CD2 | 300C11 | 76CE1 | 28.25 |
| 76CD2 | 300C11 | 76ND1 | 26.53 |
| 76CD2 | 300C11 | 67SG | 80.67 |
| 60CG | 300C11 | 219CZ3 | 81.89 |
| 60CG | 300C11 | 60CB | 20.85 |
| 60CG | 300C11 | 62CG | 92.45 |
| 60CG | 300C11 | 60CD | 16.94 |
| 60CG | 300C11 | 62CB | 74.10 |
| 60CG | 300C11 | 60OE1 | 29.42 |
| 76NE2 | 300C11 | 76CG | 30.56 |
| 76NE2 | 300C11 | 76CE1 | 15.73 |
| 76NE2 | 300C11 | 76ND1 | 26.55 |
| 76NE2 | 300C11 | 67SG | 84.24 |
| 62CD1 | 300C11 | 219CZ3 | 55.30 |
| 62CD1 | 300C11 | 60CB | 85.01 |
| 62CD1 | 300C11 | 76CG | 91.20 |
| 62CD1 | 300C11 | 219CE3 | 48.79 |
| 62CD1 | 300C11 | 62CE1 | 17.31 |
| 62CD1 | 300C11 | 62CG | 16.67 |
| 62CD1 | 300C11 | 67SG | 63.98 |
| 62CD1 | 300C11 | 62CB | 31.83 |
| 219CZ3 | 300C11 | 60CB | 62.77 |
| 219CZ3 | 300C11 | 219CE3 | 18.54 |
| 219CZ3 | 300C11 | 62CE1 | 62.87 |
| 219CZ3 | 300C11 | 62CG | 64.74 |
| 219CZ3 | 300C11 | 60CD | 84.31 |
| 219CZ3 | 300C11 | 62CB | 62.83 |
| 219CZ3 | 300C11 | 60OE1 | 75.66 |
| 60CB | 300C11 | 219CE3 | 80.87 |
| 60CB | 300C11 | 62CG | 76.95 |
| 60CB | 300C11 | 60CD | 32.56 |
| 60CB | 300C11 | 62CB | 59.19 |
| 60CB | 300C11 | 60OE1 | 37.72 |
| 76CG | 300C11 | 219CE3 | 85.39 |
| 76CG | 300C11 | 62CE1 | 73.99 |
| 76CG | 300C11 | 76CE1 | 28.49 |
| 76CG | 300C11 | 76ND1 | 16.49 |
| 76CG | 300C11 | 67SG | 63.03 |
| 219CE3 | 300C11 | 62CE1 | 50.69 |
| 219CE3 | 300C11 | 62CG | 62.59 |
| 219CE3 | 300C11 | 62CB | 67.18 |
| 219CE3 | 300C11 | 60OE1 | 92.58 |
| 62CE1 | 300C11 | 62CG | 30.57 |
| 62CE1 | 300C11 | 76ND1 | 84.08 |
| 62CE1 | 300C11 | 67SG | 54.56 |
| 62CE1 | 300C11 | 62CB | 48.06 |
| 76CE1 | 300C11 | 76ND1 | 16.15 |
| 76CE1 | 300C11 | 67SG | 70.09 |
| 62CG | 300C11 | 67SG | 61.11 |
| 62CG | 300C11 | 62CB | 18.35 |
| 60CD | 300C11 | 62CB | 90.17 |
| 60CD | 300C11 | 60OE1 | 15.00 |
| 76ND1 | 300C11 | 67SG | 58.10 |
| 67SG | 300C11 | 62CB | 74.33 |
| 62CB | 300C11 | 60OE1 | 96.80 |
| 60OE1 | 300C12 | 60CD | 18.90 |
| 60OE1 | 300C12 | 60CG | 35.99 |
| 60OE1 | 300C12 | 60OE2 | 28.67 |
| 60OE1 | 300C12 | 175CE1 | 96.27 |
| 60OE1 | 300C12 | 60CB | 36.55 |
| 76NE2 | 300C12 | 76CD2 | 18.32 |
| 76NE2 | 300C12 | 76CE1 | 13.70 |
| 76NE2 | 300C12 | 175NE2 | 98.52 |
| 60CD | 300C12 | 60CG | 22.11 |
| 60CD | 300C12 | 60OE2 | 14.58 |
| 60CD | 300C12 | 175CE1 | 86.18 |
| 60CD | 300C12 | 175NE2 | 96.96 |
| 60CD | 300C12 | 60CB | 30.42 |
| 60CG | 300C12 | 60OE2 | 31.63 |
| 60CG | 300C12 | 175CE1 | 95.29 |
| 60CG | 300C12 | 60CB | 15.14 |
| 76CD2 | 300C12 | 76CE1 | 28.36 |
| 60OE2 | 300C12 | 175CE1 | 71.64 |
| 60OE2 | 300C12 | 175NE2 | 82.51 |
| 60OE2 | 300C12 | 60CB | 43.37 |
| 76CE1 | 300C12 | 175CE1 | 95.45 |
| 76CE1 | 300C12 | 175NE2 | 90.57 |
| 175CE1 | 300C12 | 175NE2 | 16.13 |
| 219CZ3 | 300C13 | 60CG | 98.24 |
| 219CZ3 | 300C13 | 219CE3 | 21.41 |
| 219CZ3 | 300C13 | 60CB | 74.89 |
| 219CZ3 | 300C13 | 60OE1 | 98.52 |
| 219CZ3 | 300C13 | 62CD1 | 55.11 |
| 219CZ3 | 300C13 | 219CH2 | 10.78 |
| 60CG | 300C13 | 60CB | 23.43 |
| 60CG | 300C13 | 60OE1 | 36.93 |
| 60CG | 300C13 | 60CD | 21.17 |
| 60CG | 300C13 | 62CD1 | 98.17 |
| 60CG | 300C13 | 219CH2 | 87.47 |
| 219CE3 | 300C13 | 60CB | 94.41 |
| 219CE3 | 300C13 | 76CD2 | 87.88 |
| 219CE3 | 300C13 | 62CD1 | 47.95 |
| 219CE3 | 300C13 | 219CH2 | 31.58 |
| 219CE3 | 300C13 | 76CG | 84.51 |
| 60CB | 300C13 | 60OE1 | 45.48 |
| 60CB | 300C13 | 60CD | 37.22 |
| 60CB | 300C13 | 62CD1 | 83.32 |
| 60CB | 300C13 | 219CH2 | 64.11 |
| 60OE1 | 300C13 | 60CD | 17.57 |
| 60OE1 | 300C13 | 219CH2 | 89.96 |
| 76CD2 | 300C13 | 76NE2 | 18.87 |
| 76CD2 | 300C13 | 62CD1 | 90.26 |
| 76CD2 | 300C13 | 76CG | 13.57 |
| 60CD | 300C13 | 219CH2 | 94.69 |
| 76NE2 | 300C13 | 76CG | 26.99 |
| 62CD1 | 300C13 | 219CH2 | 56.52 |
| 62CD1 | 300C13 | 76CG | 78.23 |
| 60OE1 | 300C14 | 60CG | 40.01 |
| 60OE1 | 300C14 | 60CD | 19.48 |
| 60OE1 | 300C14 | 60CB | 44.84 |
| 60OE1 | 300C14 | 219CZ3 | 92.59 |
| 60OE1 | 300C14 | 60OE2 | 24.76 |
| 60CG | 300C14 | 60CD | 23.87 |
| 60CG | 300C14 | 60CB | 20.73 |
| 60CG | 300C14 | 219CZ3 | 81.80 |
| 60CG | 300C14 | 60OE2 | 30.39 |
| 60CG | 300C14 | 219CE3 | 94.66 |
| 60CD | 300C14 | 60CB | 36.97 |
| 60CD | 300C14 | 219CZ3 | 95.82 |
| 60CD | 300C14 | 60OE2 | 10.83 |
| 76NE2 | 300C14 | 76CD2 | 18.94 |
| 76NE2 | 300C14 | 219CE3 | 91.26 |
| 76CD2 | 300C14 | 219CZ3 | 88.62 |
| 76CD2 | 300C14 | 219CE3 | 72.36 |
| 60CB | 300C14 | 219CZ3 | 61.59 |
| 60CB | 300C14 | 60OE2 | 46.67 |
| 60CB | 300C14 | 219CE3 | 75.50 |
| 219CZ3 | 300C14 | 219CE3 | 16.30 |
| 104OD2 | 253ZN | 168NE2 | 98.10 |
| 104OD2 | 253ZN | 233OE2 | 79.80 |
| 104OD2 | 253ZN | 168CE1 | 74.01 |
| 104OD2 | 253ZN | 104CG | 14.80 |
| 104OD2 | 253ZN | 233CD | 76.43 |
| 104OD2 | 253ZN | 104OD1 | 34.58 |
| 104OD2 | 253ZN | 233OE1 | 78.09 |
| 104OD2 | 253ZN | 168ND1 | 82.40 |
| 104OD2 | 253ZN | 104CB | 7.30 |
| 104OD2 | 253ZN | 233CG | 73.89 |
| 104OD2 | 253ZN | 93OD2 | 92.73 |
| 168NE2 | 253ZN | 202OE1 | 88.36 |
| 168NE2 | 253ZN | 168CE1 | 24.30 |
| 168NE2 | 253ZN | 168CD2 | 18.22 |
| 168NE2 | 253ZN | 168ND1 | 18.64 |
| 168NE2 | 253ZN | 168CG | 12.73 |
| 233OE2 | 253ZN | 202OE1 | 73.52 |
| 233OE2 | 253ZN | 104CG | 71.23 |
| 233OE2 | 253ZN | 202CD | 53.63 |
| 233OE2 | 253ZN | 233CD | 15.48 |
| 233OE2 | 253ZN | 202OE2 | 49.42 |
| 233OE2 | 253ZN | 104OD1 | 67.24 |
| 233OE2 | 253ZN | 233OE1 | 35.31 |
| 233OE2 | 253ZN | 104CB | 72.59 |
| 233OE2 | 253ZN | 202CG | 47.04 |
| 233OE2 | 253ZN | 233CG | 7.48 |
| 233OE2 | 253ZN | 93OD2 | 67.16 |
| 202OE1 | 253ZN | 202CD | 21.74 |
| 202OE1 | 253ZN | 168CD2 | 70.17 |
| 202OE1 | 253ZN | 233CD | 81.57 |
| 202OE1 | 253ZN | 202OE2 | 39.94 |
| 202OE1 | 253ZN | 233OE1 | 88.54 |
| 202OE1 | 253ZN | 168ND1 | 96.73 |
| 202OE1 | 253ZN | 168CG | 79.57 |
| 202OE1 | 253ZN | 202CG | 27.09 |
| 202OE1 | 253ZN | 233CG | 80.48 |
| 202OE1 | 253ZN | 93OD2 | 90.92 |
| 168CE1 | 253ZN | 104CG | 88.81 |
| 168CE1 | 253ZN | 168CD2 | 40.57 |
| 168CE1 | 253ZN | 168ND1 | 10.75 |
| 168CE1 | 253ZN | 168CG | 28.46 |
| 168CE1 | 253ZN | 104CB | 77.60 |
| 104CG | 253ZN | 233CD | 64.96 |
| 104CG | 253ZN | 104OD1 | 20.25 |
| 104CG | 253ZN | 233OE1 | 64.01 |
| 104CG | 253ZN | 168ND1 | 97.12 |
| 104CG | 253ZN | 104CB | 13.04 |
| 104CG | 253ZN | 233CG | 64.48 |
| 104CG | 253ZN | 93OD2 | 78.31 |
| 202CD | 253ZN | 168CD2 | 90.35 |
| 202CD | 253ZN | 233CD | 60.09 |
| 202CD | 253ZN | 202OE2 | 21.49 |
| 202CD | 253ZN | 233OE1 | 67.19 |
| 202CD | 253ZN | 168CG | 97.86 |
| 202CD | 253ZN | 202CG | 14.34 |
| 202CD | 253ZN | 233CG | 60.08 |
| 202CD | 253ZN | 93OD2 | 75.41 |
| 168CD2 | 253ZN | 168ND1 | 31.80 |
| 168CD2 | 253ZN | 168CG | 13.72 |
| 168CD2 | 253ZN | 202CG | 86.59 |
| 233CD | 253ZN | 202OE2 | 49.27 |
| 233CD | 253ZN | 104OD1 | 56.27 |
| 233CD | 253ZN | 233OE1 | 20.07 |
| 233CD | 253ZN | 104CB | 69.79 |
| 233CD | 253ZN | 202CG | 57.26 |
| 233CD | 253ZN | 233CG | 10.68 |
| 233CD | 253ZN | 93OD2 | 52.92 |
| 202OE2 | 253ZN | 104OD1 | 98.59 |
| 202OE2 | 253ZN | 233OE1 | 49.82 |
| 202OE2 | 253ZN | 202CG | 32.25 |
| 202OE2 | 253ZN | 233CG | 53.59 |
| 202OE2 | 253ZN | 93OD2 | 53.93 |
| 104OD1 | 253ZN | 233OE1 | 48.85 |
| 104OD1 | 253ZN | 104CB | 33.13 |
| 104OD1 | 253ZN | 233CG | 59.78 |
| 104OD1 | 253ZN | 93OD2 | 58.16 |
| 233OE1 | 253ZN | 104CB | 72.97 |
| 233OE1 | 253ZN | 202CG | 69.26 |
| 233OE1 | 253ZN | 233CG | 30.58 |
| 233OE1 | 253ZN | 93OD2 | 33.44 |
| 168ND1 | 253ZN | 168CG | 18.68 |
| 168ND1 | 253ZN | 104CB | 85.16 |
| 168CG | 253ZN | 202CG | 91.25 |
| 104CB | 253ZN | 233CG | 66.80 |
| 104CB | 253ZN | 93OD2 | 90.83 |
| 202CG | 253ZN | 233CG | 54.29 |
| 202CG | 253ZN | 93OD2 | 84.78 |
| 233CG | 253ZN | 93OD2 | 63.60 |
| 233OE1 | 254ZN | 104OD1 | 93.69 |
| 233OE1 | 254ZN | 93OD2 | 84.63 |
| 233OE1 | 254ZN | 233CD | 20.60 |
| 233OE1 | 254ZN | 104CG | 89.10 |
| 233OE1 | 254ZN | 233OE2 | 41.97 |
| 233OE1 | 254ZN | 104OD2 | 87.96 |
| 233OE1 | 254ZN | 202OE2 | 46.39 |
| 233OE1 | 254ZN | 233CG | 17.35 |
| 233OE1 | 254ZN | 104CB | 87.62 |
| 233OE1 | 254ZN | 93CA | 99.36 |
| 233OE1 | 254ZN | 104C | 70.88 |
| 233OE1 | 254ZN | 233CB | 9.58 |
| 233OE1 | 254ZN | 202CD | 49.53 |
| 233OE1 | 254ZN | 104CA | 86.84 |
| 233OE1 | 254ZN | 202OE1 | 62.68 |
| 93OD1 | 254ZN | 93OD2 | 63.70 |
| 93OD1 | 254ZN | 93CG | 32.03 |
| 93OD1 | 254ZN | 93CB | 30.19 |
| 93OD1 | 254ZN | 93CA | 19.84 |
| 93OD1 | 254ZN | 104C | 94.80 |
| 93OD1 | 254ZN | 104CA | 95.80 |
| 93OD1 | 254ZN | 93C | 3.92 |
| 93OD1 | 254ZN | 95CB | 70.60 |
| 104OD1 | 254ZN | 233CD | 79.55 |
| 104OD1 | 254ZN | 104CG | 18.77 |
| 104OD1 | 254ZN | 233OE2 | 75.53 |
| 104OD1 | 254ZN | 104OD2 | 40.61 |
| 104OD1 | 254ZN | 233CG | 76.43 |
| 104OD1 | 254ZN | 104CB | 10.96 |
| 104OD1 | 254ZN | 104C | 29.02 |
| 104OD1 | 254ZN | 233CB | 90.96 |
| 104OD1 | 254ZN | 104CA | 11.53 |
| 104OD1 | 254ZN | 95CB | 43.85 |
| 93OD2 | 254ZN | 93CG | 31.67 |
| 93OD2 | 254ZN | 93CB | 33.54 |
| 93OD2 | 254ZN | 202OE2 | 75.68 |
| 93OD2 | 254ZN | 93CA | 47.98 |
| 93OD2 | 254ZN | 233CB | 84.34 |
| 93OD2 | 254ZN | 202CD | 87.76 |
| 93OD2 | 254ZN | 93C | 64.01 |
| 93OD2 | 254ZN | 202OE1 | 95.40 |
| 93CG | 254ZN | 93CB | 1.98 |
| 93CG | 254ZN | 93CA | 18.97 |
| 93CG | 254ZN | 233CB | 97.17 |
| 93CG | 254ZN | 93C | 32.54 |
| 233CD | 254ZN | 104CG | 71.07 |
| 233CD | 254ZN | 233OE2 | 22.94 |
| 233CD | 254ZN | 104OD2 | 67.50 |
| 233CD | 254ZN | 202OE2 | 45.24 |
| 233CD | 254ZN | 233CG | 13.34 |
| 233CD | 254ZN | 104CB | 71.45 |
| 233CD | 254ZN | 104C | 63.36 |
| 233CD | 254ZN | 233CB | 26.67 |
| 233CD | 254ZN | 202CD | 42.29 |
| 233CD | 254ZN | 104CA | 75.58 |
| 233CD | 254ZN | 202OE1 | 51.88 |
| 104CG | 254ZN | 233OE2 | 61.18 |
| 104CG | 254ZN | 104OD2 | 22.04 |
| 104CG | 254ZN | 233CG | 72.17 |
| 104CG | 254ZN | 104CB | 9.19 |
| 104CG | 254ZN | 104C | 40.08 |
| 104CG | 254ZN | 233CB | 89.42 |
| 104CG | 254ZN | 202CD | 88.42 |
| 104CG | 254ZN | 104CA | 27.56 |
| 104CG | 254ZN | 95CB | 55.21 |
| 104CG | 254ZN | 202OE1 | 81.96 |
| 233OE2 | 254ZN | 104OD2 | 50.18 |
| 233OE2 | 254ZN | 202OE2 | 40.80 |
| 233OE2 | 254ZN | 233CG | 35.30 |
| 233OE2 | 254ZN | 104CB | 65.17 |
| 233OE2 | 254ZN | 104C | 70.48 |
| 233OE2 | 254ZN | 233CB | 49.35 |
| 233OE2 | 254ZN | 202CD | 31.54 |
| 233OE2 | 254ZN | 104CA | 76.20 |
| 233OE2 | 254ZN | 202OE1 | 34.78 |
| 104OD2 | 254ZN | 202OE2 | 83.53 |
| 104OD2 | 254ZN | 233CG | 73.76 |
| 104OD2 | 254ZN | 104CB | 31.07 |
| 104OD2 | 254ZN | 104C | 59.63 |
| 104OD2 | 254ZN | 233CB | 91.85 |
| 104OD2 | 254ZN | 202CD | 71.08 |
| 104OD2 | 254ZN | 104CA | 49.47 |
| 104OD2 | 254ZN | 95CB | 68.60 |
| 104OD2 | 254ZN | 202OE1 | 62.07 |
| 93CB | 254ZN | 93CA | 17.95 |
| 93CB | 254ZN | 233CB | 98.50 |
| 93CB | 254ZN | 93C | 30.80 |
| 93CB | 254ZN | 95CB | 98.63 |
| 202OE2 | 254ZN | 233CG | 56.03 |
| 202OE2 | 254ZN | 233CB | 55.62 |
| 202OE2 | 254ZN | 202CD | 12.51 |
| 202OE2 | 254ZN | 202OE1 | 25.03 |
| 233CG | 254ZN | 104CB | 70.30 |
| 233CG | 254ZN | 104C | 55.26 |
| 233CG | 254ZN | 233CB | 18.14 |
| 233CG | 254ZN | 202CD | 54.84 |
| 233CG | 254ZN | 104CA | 70.12 |
| 233CG | 254ZN | 202OE1 | 65.14 |
| 104CB | 254ZN | 104C | 31.57 |
| 104CB | 254ZN | 233CB | 86.46 |
| 104CB | 254ZN | 202CD | 94.35 |
| 104CB | 254ZN | 104CA | 18.41 |
| 104CB | 254ZN | 95CB | 52.61 |
| 104CB | 254ZN | 202OE1 | 89.34 |
| 93CA | 254ZN | 233CB | 92.20 |
| 93CA | 254ZN | 93C | 18.17 |
| 93CA | 254ZN | 95CB | 89.97 |
| 104C | 254ZN | 233CB | 65.66 |
| 104C | 254ZN | 104CA | 18.20 |
| 104C | 254ZN | 93C | 92.10 |
| 104C | 254ZN | 95CB | 64.34 |
| 233CB | 254ZN | 202CD | 59.11 |
| 233CB | 254ZN | 104CA | 82.75 |
| 233CB | 254ZN | 202OE1 | 72.20 |
| 202CD | 254ZN | 202OE1 | 14.45 |
| 104CA | 254ZN | 93C | 94.12 |
| 104CA | 254ZN | 95CB | 48.74 |
| 93C | 254ZN | 95CB | 71.81 |

TABLE VIII
Provides a three dimensional protein coordinate set of an S. pneumoniae
methionine aminopeptidase crystalline structure.

| | | | | |
|---|---|---|---|---|
| Residue Atom | X | Y | Z | B |

| | | | | | | |
|---|---|---|---|---|---|---|
| 2 | ILE | CB | 10.87 | 40.59 | 15.61 | 18.51 |
| 2 | ILE | CG2 | 11.88 | 41.49 | 16.31 | 18.53 |
| 2 | ILE | CG1 | 9.48 | 41.23 | 15.62 | 18.55 |
| 2 | ILE | CD1 | 8.91 | 41.42 | 17.02 | 18.56 |
| 2 | ILE | C | 12.69 | 39.66 | 14.19 | 18.35 |
| 2 | ILE | O | 12.84 | 38.57 | 14.74 | 18.43 |
| 2 | ILE | N | 10.34 | 39.47 | 13.43 | 18.53 |
| 2 | ILE | CA | 11.32 | 40.33 | 14.16 | 18.45 |
| 3 | THR | N | 13.68 | 40.32 | 13.60 | 18.20 |
| 3 | THR | CA | 15.03 | 39.77 | 13.60 | 17.99 |
| 3 | THR | CB | 15.75 | 40.02 | 12.25 | 18.05 |
| 3 | THR | OG1 | 15.92 | 41.43 | 12.05 | 18.08 |
| 3 | THR | CG2 | 14.95 | 39.44 | 11.10 | 18.08 |
| 3 | THR | C | 15.86 | 40.40 | 14.70 | 17.77 |
| 3 | THR | O | 15.71 | 41.58 | 15.01 | 17.79 |
| 4 | LEU | N | 16.73 | 39.59 | 15.31 | 17.46 |
| 4 | LEU | CA | 17.62 | 40.07 | 16.35 | 17.13 |
| 4 | LEU | CB | 17.71 | 39.05 | 17.48 | 17.20 |
| 4 | LEU | CG | 16.37 | 38.76 | 18.18 | 17.22 |
| 4 | LEU | CD1 | 16.59 | 37.74 | 19.29 | 17.29 |
| 4 | LEU | CD2 | 15.79 | 40.04 | 18.74 | 17.25 |
| 4 | LEU | C | 18.95 | 40.22 | 15.62 | 16.87 |
| 4 | LEU | O | 19.55 | 39.24 | 15.18 | 16.89 |
| 5 | LYS | N | 19.39 | 41.46 | 15.47 | 16.51 |
| 5 | LYS | CA | 20.60 | 41.79 | 14.74 | 16.14 |
| 5 | LYS | CB | 20.71 | 43.30 | 14.58 | 16.07 |
| 5 | LYS | CG | 19.79 | 43.89 | 13.52 | 15.97 |
| 5 | LYS | CD | 18.32 | 43.63 | 13.82 | 15.88 |
| 5 | LYS | CE | 17.43 | 44.27 | 12.77 | 15.83 |
| 5 | LYS | NZ | 15.98 | 43.99 | 12.99 | 15.72 |
| 5 | LYS | C | 21.94 | 41.25 | 15.23 | 15.94 |
| 5 | LYS | O | 22.20 | 41.12 | 16.43 | 15.92 |
| 6 | SER | N | 22.80 | 40.95 | 14.25 | 15.67 |
| 6 | SER | CA | 24.14 | 40.46 | 14.49 | 15.40 |
| 6 | SER | CB | 24.62 | 39.65 | 13.28 | 15.40 |
| 6 | SER | OG | 24.72 | 40.50 | 12.15 | 15.33 |
| 6 | SER | C | 25.04 | 41.67 | 14.66 | 15.19 |
| 6 | SER | O | 24.62 | 42.80 | 14.38 | 15.07 |
| 7 | ALA | N | 26.27 | 41.45 | 15.09 | 14.97 |
| 7 | ALA | CA | 27.21 | 42.55 | 15.27 | 14.71 |
| 7 | ALA | CB | 28.53 | 42.03 | 15.81 | 14.86 |
| 7 | ALA | C | 27.42 | 43.24 | 13.92 | 14.48 |
| 7 | ALA | O | 27.52 | 44.47 | 13.85 | 14.38 |
| 8 | ARG | N | 27.49 | 42.45 | 12.86 | 14.19 |
| 8 | ARG | CA | 27.69 | 42.98 | 11.51 | 13.88 |
| 8 | ARG | CB | 27.81 | 41.83 | 10.50 | 14.13 |
| 8 | ARG | CG | 27.96 | 42.28 | 9.05 | 14.47 |
| 8 | ARG | CD | 28.14 | 41.09 | 8.12 | 14.85 |
| 8 | ARG | NE | 29.39 | 40.37 | 8.39 | 15.19 |
| 8 | ARG | CZ | 29.74 | 39.23 | 7.79 | 15.38 |
| 8 | ARG | NH1 | 28.94 | 38.68 | 6.89 | 15.52 |
| 8 | ARG | NH2 | 30.89 | 38.65 | 8.11 | 15.58 |
| 8 | ARG | C | 26.56 | 43.92 | 11.10 | 13.54 |
| 8 | ARG | O | 26.79 | 45.01 | 10.58 | 13.34 |
| 9 | GLU | N | 25.32 | 43.49 | 11.33 | 13.23 |
| 9 | GLU | CA | 24.16 | 44.29 | 10.97 | 13.00 |
| 9 | GLU | CB | 22.88 | 43.46 | 11.13 | 13.22 |
| 9 | GLU | CG | 22.86 | 42.25 | 10.20 | 13.63 |
| 9 | GLU | CD | 21.73 | 41.28 | 10.47 | 13.85 |
| 9 | GLU | OE1 | 21.37 | 41.10 | 11.66 | 13.95 |
| 9 | GLU | OE2 | 21.20 | 40.68 | 9.51 | 14.15 |
| 9 | GLU | C | 24.08 | 45.58 | 11.80 | 12.70 |
| 9 | GLU | O | 23.72 | 46.63 | 11.29 | 12.42 |
| 10 | ILE | N | 24.43 | 45.48 | 13.08 | 12.55 |
| 10 | ILE | CA | 24.41 | 46.64 | 13.96 | 12.52 |
| 10 | ILE | CB | 24.64 | 46.23 | 15.44 | 12.69 |
| 10 | ILE | CG2 | 24.78 | 47.47 | 16.31 | 12.79 |
| 10 | ILE | CG1 | 23.47 | 45.36 | 15.92 | 12.88 |
| 10 | ILE | CD1 | 23.68 | 44.76 | 17.30 | 13.16 |
| 10 | ILE | C | 25.47 | 47.66 | 13.53 | 12.30 |
| 10 | ILE | O | 25.22 | 48.86 | 13.55 | 12.22 |
| 11 | GLU | N | 26.65 | 47.17 | 13.15 | 12.14 |
| 11 | GLU | CA | 27.71 | 48.06 | 12.71 | 11.97 |
| 11 | GLU | CB | 28.99 | 47.28 | 12.39 | 12.31 |
| 11 | GLU | CG | 30.10 | 48.16 | 11.81 | 12.85 |
| 11 | GLU | CD | 31.37 | 47.40 | 11.48 | 13.15 |
| 11 | GLU | OE1 | 31.38 | 46.16 | 11.60 | 13.44 |
| 11 | GLU | OE2 | 32.36 | 48.05 | 11.08 | 13.40 |
| 11 | GLU | C | 27.28 | 48.85 | 11.47 | 11.62 |
| 11 | GLU | O | 27.51 | 50.05 | 11.38 | 11.50 |
| 12 | ALA | N | 26.65 | 48.16 | 10.53 | 11.28 |
| 12 | ALA | CA | 26.18 | 48.82 | 9.31 | 10.93 |
| 12 | ALA | CB | 25.57 | 47.79 | 8.36 | 10.97 |
| 12 | ALA | C | 25.16 | 49.90 | 9.66 | 10.68 |
| 12 | ALA | O | 25.21 | 51.00 | 9.11 | 10.51 |
| 13 | MET | N | 24.25 | 49.61 | 10.58 | 10.59 |
| 13 | MET | CA | 23.25 | 50.59 | 10.98 | 10.55 |
| 13 | MET | CB | 22.20 | 49.97 | 11.89 | 10.71 |
| 13 | MET | CG | 21.29 | 48.99 | 11.21 | 11.13 |
| 13 | MET | SD | 20.14 | 48.33 | 12.41 | 11.37 |
| 13 | MET | CE | 18.89 | 47.63 | 11.34 | 11.74 |
| 13 | MET | C | 23.89 | 51.77 | 11.71 | 10.43 |
| 13 | MET | O | 23.49 | 52.92 | 11.52 | 10.31 |
| 14 | ASP | N | 24.87 | 51.48 | 12.55 | 10.50 |
| 14 | ASP | CA | 25.54 | 52.52 | 13.31 | 10.60 |
| 14 | ASP | CB | 26.56 | 51.90 | 14.27 | 11.07 |
| 14 | ASP | CG | 27.13 | 52.90 | 15.25 | 11.47 |
| 14 | ASP | OD1 | 26.38 | 53.78 | 15.71 | 11.60 |
| 14 | ASP | OD2 | 28.33 | 52.79 | 15.57 | 12.16 |
| 14 | ASP | C | 26.21 | 53.52 | 12.36 | 10.37 |
| 14 | ASP | O | 26.18 | 54.73 | 12.59 | 10.27 |
| 15 | LYS | N | 26.80 | 53.00 | 11.28 | 10.20 |
| 15 | LYS | CA | 27.46 | 53.86 | 10.30 | 10.14 |
| 15 | LYS | CB | 28.25 | 53.01 | 9.31 | 10.33 |
| 15 | LYS | CG | 29.49 | 52.35 | 9.89 | 10.62 |
| 15 | LYS | CD | 30.08 | 51.38 | 8.89 | 10.95 |
| 15 | LYS | CE | 31.35 | 50.74 | 9.40 | 11.14 |
| 15 | LYS | NZ | 31.88 | 49.77 | 8.40 | 11.26 |
| 15 | LYS | C | 26.44 | 54.71 | 9.55 | 9.97 |
| 15 | LYS | O | 26.64 | 55.91 | 9.36 | 9.78 |
| 16 | ALA | N | 25.35 | 54.09 | 9.12 | 9.82 |
| 16 | ALA | CA | 24.31 | 54.83 | 8.41 | 9.73 |
| 16 | ALA | CB | 23.20 | 53.88 | 7.95 | 9.77 |
| 16 | ALA | C | 23.75 | 55.90 | 9.33 | 9.62 |
| 16 | ALA | O | 23.46 | 57.02 | 8.90 | 9.49 |
| 17 | GLY | N | 23.61 | 55.56 | 10.61 | 9.52 |
| 17 | GLY | CA | 23.09 | 56.50 | 11.59 | 9.61 |
| 17 | GLY | C | 24.04 | 57.66 | 11.86 | 9.59 |
| 17 | GLY | O | 23.60 | 58.77 | 12.14 | 9.47 |
| 18 | ASP | N | 25.34 | 57.39 | 11.79 | 9.70 |
| 18 | ASP | CA | 26.34 | 58.44 | 11.99 | 9.83 |
| 18 | ASP | CB | 27.75 | 57.86 | 12.03 | 10.29 |
| 18 | ASP | CG | 28.09 | 57.21 | 13.36 | 10.61 |
| 18 | ASP | OD1 | 27.28 | 57.28 | 14.31 | 10.72 |
| 18 | ASP | OD2 | 29.19 | 56.62 | 13.46 | 11.34 |
| 18 | ASP | C | 26.24 | 59.44 | 10.84 | 9.77 |
| 18 | ASP | O | 26.36 | 60.65 | 11.03 | 9.70 |
| 19 | PHE | N | 26.04 | 58.92 | 9.63 | 9.74 |
| 19 | PHE | CA | 25.92 | 59.78 | 8.45 | 9.74 |
| 19 | PHE | CB | 25.88 | 58.94 | 7.17 | 10.16 |
| 19 | PHE | CG | 25.61 | 59.73 | 5.93 | 10.66 |
| 19 | PHE | CD1 | 26.47 | 60.75 | 5.51 | 10.96 |
| 19 | PHE | CD2 | 24.48 | 59.47 | 5.17 | 10.87 |
| 19 | PHE | CE1 | 26.20 | 61.48 | 4.36 | 11.24 |
| 19 | PHE | CE2 | 24.20 | 60.20 | 4.01 | 11.18 |
| 19 | PHE | CZ | 25.07 | 61.21 | 3.61 | 11.28 |
| 19 | PHE | C | 24.65 | 60.63 | 8.57 | 9.51 |
| 19 | PHE | O | 24.68 | 61.84 | 8.35 | 9.38 |
| 20 | LEU | N | 23.54 | 59.99 | 8.93 | 9.33 |
| 20 | LEU | CA | 22.28 | 60.70 | 9.09 | 9.22 |
| 20 | LEU | CB | 21.16 | 59.72 | 9.47 | 9.35 |
| 20 | LEU | CG | 19.72 | 60.15 | 9.21 | 9.46 |
| 20 | LEU | CD1 | 18.80 | 58.95 | 9.31 | 9.54 |
| 20 | LEU | CD2 | 19.30 | 61.24 | 10.19 | 9.65 |
| 20 | LEU | C | 22.45 | 61.79 | 10.16 | 9.13 |
| 20 | LEU | O | 22.04 | 62.93 | 9.97 | 8.91 |
| 21 | ALA | N | 23.09 | 61.43 | 11.27 | 9.20 |
| 21 | ALA | CA | 23.32 | 62.40 | 12.34 | 9.31 |
| 21 | ALA | CB | 24.02 | 61.73 | 13.52 | 9.48 |
| 21 | ALA | C | 24.16 | 63.58 | 11.84 | 9.43 |
| 21 | ALA | O | 23.91 | 64.73 | 12.22 | 9.41 |
| 22 | SER | N | 25.14 | 63.31 | 10.98 | 9.59 |
| 22 | SER | CA | 25.98 | 64.38 | 10.45 | 9.86 |
| 22 | SER | CB | 27.17 | 63.81 | 9.65 | 10.10 |
| 22 | SER | OG | 26.79 | 63.37 | 8.36 | 10.99 |
| 22 | SER | C | 25.16 | 65.32 | 9.59 | 9.78 |
| 22 | SER | O | 25.44 | 66.52 | 9.51 | 9.83 |
| 23 | ILE | N | 24.13 | 64.79 | 8.93 | 9.73 |
| 23 | ILE | CA | 23.26 | 65.63 | 8.11 | 9.74 |
| 23 | ILE | CB | 22.34 | 64.76 | 7.21 | 9.95 |
| 23 | ILE | CG2 | 21.18 | 65.58 | 6.67 | 10.00 |
| 23 | ILE | CG1 | 23.18 | 64.17 | 6.07 | 10.15 |
| 23 | ILE | CD1 | 22.41 | 63.25 | 5.16 | 10.47 |
| 23 | ILE | C | 22.45 | 66.54 | 9.03 | 9.62 |
| 23 | ILE | O | 22.31 | 67.73 | 8.75 | 9.53 |
| 24 | HIS | N | 21.93 | 66.00 | 10.13 | 9.61 |
| 24 | HIS | CA | 21.19 | 66.84 | 11.07 | 9.63 |
| 24 | HIS | CB | 20.61 | 66.01 | 12.21 | 9.44 |
| 24 | HIS | CG | 19.29 | 65.39 | 11.89 | 9.18 |
| 24 | HIS | CD2 | 18.85 | 64.12 | 11.97 | 9.16 |
| 24 | HIS | ND1 | 18.23 | 66.13 | 11.40 | 9.17 |
| 24 | HIS | CE1 | 17.19 | 65.33 | 11.21 | 9.08 |
| 24 | HIS | NE2 | 17.55 | 64.11 | 11.54 | 9.09 |
| 24 | HIS | C | 22.10 | 67.94 | 11.62 | 9.77 |
| 24 | HIS | O | 21.68 | 69.09 | 11.76 | 9.77 |
| 25 | ILE | N | 23.35 | 67.61 | 11.91 | 10.02 |
| 25 | ILE | CA | 24.28 | 68.61 | 12.42 | 10.39 |
| 25 | ILE | CB | 25.65 | 67.97 | 12.78 | 10.65 |
| 25 | ILE | CG2 | 26.67 | 69.06 | 13.07 | 10.81 |
| 25 | ILE | CG1 | 25.47 | 67.06 | 13.99 | 10.87 |
| 25 | ILE | CD1 | 26.66 | 66.16 | 14.27 | 11.21 |
| 25 | ILE | C | 24.45 | 69.71 | 11.37 | 10.48 |
| 25 | ILE | O | 24.47 | 70.90 | 11.70 | 10.46 |
| 26 | GLY | N | 24.55 | 69.32 | 10.10 | 10.67 |
| 26 | GLY | CA | 24.70 | 70.31 | 9.04 | 10.94 |
| 26 | GLY | C | 23.46 | 71.17 | 8.87 | 11.09 |
| 26 | GLY | O | 23.55 | 72.35 | 8.55 | 11.09 |
| 27 | LEU | N | 22.28 | 70.57 | 9.07 | 11.28 |
| 27 | LEU | CA | 21.03 | 71.31 | 8.93 | 11.54 |
| 27 | LEU | CB | 19.83 | 70.36 | 9.04 | 11.49 |
| 27 | LEU | CG | 19.60 | 69.42 | 7.85 | 11.48 |
| 27 | LEU | CD1 | 18.44 | 68.48 | 8.16 | 11.52 |
| 27 | LEU | CD2 | 19.30 | 70.24 | 6.60 | 11.61 |
| 27 | LEU | C | 20.89 | 72.43 | 9.95 | 11.79 |
| 27 | LEU | O | 20.14 | 73.38 | 9.74 | 11.70 |
| 28 | ARG | N | 21.62 | 72.33 | 11.06 | 12.16 |
| 28 | ARG | CA | 21.57 | 73.36 | 12.09 | 12.67 |
| 28 | ARG | CB | 22.50 | 73.02 | 13.24 | 12.54 |
| 28 | ARG | CG | 22.14 | 71.74 | 13.97 | 12.43 |
| 28 | ARG | CD | 23.18 | 71.39 | 15.01 | 12.25 |
| 28 | ARG | NE | 22.93 | 70.08 | 15.60 | 12.21 |
| 28 | ARG | CZ | 23.71 | 69.50 | 16.49 | 12.20 |
| 28 | ARG | NH1 | 24.82 | 70.10 | 16.91 | 12.41 |
| 28 | ARG | NH2 | 23.40 | 68.31 | 16.97 | 12.28 |
| 28 | ARG | C | 21.97 | 74.72 | 11.52 | 13.09 |
| 28 | ARG | O | 21.43 | 75.75 | 11.92 | 13.24 |
| 29 | ASP | N | 22.92 | 74.72 | 10.59 | 13.59 |
| 29 | ASP | CA | 23.39 | 75.96 | 9.99 | 14.06 |
| 29 | ASP | CB | 24.84 | 75.81 | 9.53 | 14.57 |
| 29 | ASP | CG | 25.78 | 75.47 | 10.67 | 14.98 |
| 29 | ASP | OD1 | 25.68 | 76.11 | 11.74 | 15.40 |
| 29 | ASP | OD2 | 26.62 | 74.57 | 10.49 | 15.40 |
| 29 | ASP | C | 22.55 | 76.41 | 8.80 | 14.11 |
| 29 | ASP | O | 22.53 | 77.59 | 8.46 | 14.23 |
| 30 | LEU | N | 21.85 | 75.47 | 8.18 | 14.13 |
| 30 | LEU | CA | 21.04 | 75.77 | 7.00 | 14.14 |
| 30 | LEU | CB | 20.92 | 74.52 | 6.13 | 14.27 |
| 30 | LEU | CG | 20.13 | 74.65 | 4.83 | 14.41 |
| 30 | LEU | CD1 | 20.75 | 75.72 | 3.95 | 14.63 |
| 30 | LEU | CD2 | 20.13 | 73.31 | 4.11 | 14.60 |
| 30 | LEU | C | 19.64 | 76.30 | 7.31 | 14.07 |
| 30 | LEU | O | 19.16 | 77.21 | 6.65 | 14.15 |
| 31 | ILE | N | 19.00 | 75.73 | 8.32 | 13.96 |
| 31 | ILE | CA | 17.66 | 76.12 | 8.70 | 13.86 |
| 31 | ILE | CB | 16.98 | 74.99 | 9.51 | 13.73 |
| 31 | ILE | CG2 | 15.59 | 75.40 | 9.96 | 13.75 |
| 31 | ILE | CG1 | 16.92 | 73.73 | 8.63 | 13.62 |
| 31 | ILE | CD1 | 16.45 | 72.48 | 9.34 | 13.56 |
| 31 | ILE | C | 17.64 | 77.44 | 9.47 | 13.88 |
| 31 | ILE | O | 17.81 | 77.47 | 10.69 | 13.97 |
| 32 | LYS | N | 17.41 | 78.52 | 8.74 | 13.87 |
| 32 | LYS | CA | 17.38 | 79.87 | 9.30 | 13.83 |
| 32 | LYS | CB | 18.73 | 80.56 | 9.06 | 14.41 |
| 32 | LYS | CG | 19.93 | 79.78 | 9.52 | 15.02 |
| 32 | LYS | CD | 21.22 | 80.48 | 9.12 | 15.69 |
| 32 | LYS | CE | 21.33 | 81.85 | 9.77 | 16.03 |
| 32 | LYS | NZ | 22.55 | 82.58 | 9.32 | 16.41 |
| 32 | LYS | C | 16.30 | 80.69 | 8.61 | 13.54 |
| 32 | LYS | O | 15.83 | 80.34 | 7.53 | 13.31 |
| 33 | PRO | N | 15.88 | 81.81 | 9.23 | 13.34 |
| 33 | PRO | CD | 16.25 | 82.31 | 10.57 | 13.34 |
| 33 | PRO | CA | 14.85 | 82.65 | 8.62 | 13.22 |
| 33 | PRO | CB | 14.71 | 83.81 | 9.61 | 13.26 |
| 33 | PRO | CG | 15.05 | 83.17 | 10.93 | 13.31 |
| 33 | PRO | C | 15.30 | 83.13 | 7.24 | 13.11 |
| 33 | PRO | O | 16.47 | 83.48 | 7.06 | 13.07 |
| 34 | GLY | N | 14.39 | 83.12 | 6.28 | 12.96 |
| 34 | GLY | CA | 14.73 | 83.57 | 4.94 | 12.87 |
| 34 | GLY | C | 15.18 | 82.49 | 3.98 | 12.72 |
| 34 | GLY | O | 15.23 | 82.71 | 2.77 | 12.83 |
| 35 | VAL | N | 15.51 | 81.32 | 4.51 | 12.53 |
| 35 | VAL | CA | 15.95 | 80.20 | 3.69 | 12.31 |
| 35 | VAL | CB | 16.86 | 79.25 | 4.49 | 12.38 |
| 35 | VAL | CG1 | 17.22 | 78.02 | 3.66 | 12.44 |
| 35 | VAL | CG2 | 18.12 | 79.98 | 4.93 | 12.47 |
| 35 | VAL | C | 14.75 | 79.43 | 3.15 | 12.10 |
| 35 | VAL | O | 13.76 | 79.25 | 3.86 | 11.96 |
| 36 | ASP | N | 14.85 | 78.98 | 1.91 | 11.90 |
| 36 | ASP | CA | 13.78 | 78.20 | 1.28 | 11.69 |
| 36 | ASP | CB | 14.02 | 78.15 | −0.24 | 11.89 |
| 36 | ASP | CG | 12.90 | 77.44 | −0.99 | 11.93 |
| 36 | ASP | OD1 | 12.04 | 76.80 | −0.35 | 11.90 |
| 36 | ASP | OD2 | 12.90 | 77.53 | −2.24 | 12.34 |
| 36 | ASP | C | 13.83 | 76.79 | 1.86 | 11.50 |
| 36 | ASP | O | 14.87 | 76.14 | 1.82 | 11.39 |
| 37 | MET | N | 12.71 | 76.33 | 2.41 | 11.32 |
| 37 | MET | CA | 12.62 | 75.00 | 3.01 | 11.22 |
| 37 | MET | CB | 11.17 | 74.70 | 3.42 | 11.61 |
| 37 | MET | CG | 10.98 | 73.37 | 4.17 | 12.20 |
| 37 | MET | SD | 9.32 | 72.62 | 3.96 | 13.13 |
| 37 | MET | CE | 8.27 | 73.79 | 4.82 | 13.09 |
| 37 | MET | C | 13.09 | 73.92 | 2.03 | 10.99 |
| 37 | MET | O | 13.63 | 72.89 | 2.44 | 10.74 |
| 38 | TRP | N | 12.89 | 74.17 | 0.74 | 10.87 |
| 38 | TRP | CA | 13.29 | 73.20 | −0.27 | 10.86 |
| 38 | TRP | CB | 12.93 | 73.72 | −1.67 | 11.05 |
| 38 | TRP | CG | 13.34 | 72.77 | −2.75 | 11.24 |
| 38 | TRP | CD2 | 12.78 | 71.48 | −3.03 | 11.40 |
| 38 | TRP | CE2 | 13.54 | 70.91 | −4.07 | 11.49 |
| 38 | TRP | CE3 | 11.71 | 70.75 | −2.50 | 11.46 |
| 38 | TRP | CD1 | 14.38 | 72.93 | −3.62 | 11.35 |
| 38 | TRP | NE1 | 14.52 | 71.81 | −4.41 | 11.42 |
| 38 | TRP | CZ2 | 13.28 | 69.64 | −4.59 | 11.61 |
| 38 | TRP | CZ3 | 11.44 | 69.48 | −3.02 | 11.61 |
| 38 | TRP | CH2 | 12.22 | 68.94 | −4.05 | 11.64 |
| 38 | TRP | C | 14.77 | 72.88 | −0.19 | 10.73 |
| 38 | TRP | O | 15.20 | 71.78 | −0.55 | 10.63 |
| 39 | GLU | N | 15.57 | 73.83 | 0.28 | 10.68 |
| 39 | GLU | CA | 17.00 | 73.60 | 0.39 | 10.72 |
| 39 | GLU | CB | 17.74 | 74.91 | 0.72 | 11.23 |
| 39 | GLU | CG | 17.73 | 75.91 | −0.43 | 12.24 |
| 39 | GLU | CD | 18.18 | 75.30 | −1.75 | 12.79 |
| 39 | GLU | OE1 | 19.20 | 74.58 | −1.76 | 13.14 |
| 39 | GLU | OE2 | 17.51 | 75.53 | −2.78 | 13.50 |
| 39 | GLU | C | 17.35 | 72.50 | 1.39 | 10.34 |
| 39 | GLU | O | 18.45 | 71.96 | 1.35 | 10.22 |
| 40 | VAL | N | 16.42 | 72.17 | 2.28 | 10.07 |
| 40 | VAL | CA | 16.65 | 71.09 | 3.24 | 9.83 |
| 40 | VAL | CB | 15.50 | 70.98 | 4.27 | 9.88 |
| 40 | VAL | CG1 | 15.60 | 69.68 | 5.05 | 9.96 |
| 40 | VAL | CG2 | 15.55 | 72.17 | 5.22 | 9.99 |
| 40 | VAL | C | 16.74 | 69.80 | 2.42 | 9.71 |
| 40 | VAL | O | 17.65 | 68.98 | 2.60 | 9.57 |
| 41 | GLU | N | 15.80 | 69.62 | 1.50 | 9.62 |
| 41 | GLU | CA | 15.77 | 68.45 | 0.63 | 9.69 |
| 41 | GLU | CB | 14.51 | 68.47 | −0.23 | 9.62 |
| 41 | GLU | CG | 14.42 | 67.40 | −1.32 | 9.47 |
| 41 | GLU | CD | 14.21 | 65.98 | −0.79 | 9.33 |
| 41 | GLU | CE1 | 13.94 | 65.81 | 0.42 | 9.36 |
| 41 | GLU | OE2 | 14.31 | 65.04 | −1.60 | 9.25 |
| 41 | GLU | C | 17.02 | 68.44 | −0.25 | 9.78 |
| 41 | GLU | O | 17.65 | 67.39 | −0.43 | 9.69 |
| 42 | GLU | N | 17.39 | 69.59 | −0.80 | 9.95 |
| 42 | GLU | CA | 18.57 | 69.67 | −1.65 | 10.21 |
| 42 | GLU | CB | 18.72 | 71.08 | −2.24 | 10.72 |
| 42 | GLU | CG | 17.59 | 71.45 | −3.19 | 11.61 |
| 42 | GLU | CD | 17.75 | 70.84 | −4.58 | 12.08 |
| 42 | GLU | OE1 | 18.43 | 69.81 | −4.73 | 12.51 |
| 42 | GLU | OE2 | 17.17 | 71.42 | −5.52 | 12.70 |
| 42 | GLU | C | 19.84 | 69.29 | −0.89 | 10.07 |
| 42 | GLU | O | 20.70 | 68.60 | −1.43 | 9.90 |
| 43 | TYR | N | 19.95 | 69.74 | 0.35 | 9.99 |
| 43 | TYR | CA | 21.12 | 69.43 | 1.16 | 9.95 |
| 43 | TYR | CB | 21.04 | 70.15 | 2.51 | 10.35 |
| 43 | TYR | CG | 22.19 | 69.83 | 3.44 | 10.77 |
| 43 | TYR | CD1 | 23.49 | 70.24 | 3.13 | 11.10 |
| 43 | TYR | CE1 | 24.56 | 69.94 | 3.98 | 11.45 |
| 43 | TYR | CD2 | 21.98 | 69.11 | 4.61 | 11.00 |
| 43 | TYR | CE2 | 23.04 | 68.80 | 5.46 | 11.28 |
| 43 | TYR | CZ | 24.32 | 69.21 | 5.14 | 11.41 |
| 43 | TYR | OH | 25.36 | 68.90 | 5.99 | 11.81 |
| 43 | TYR | C | 21.24 | 67.92 | 1.39 | 9.72 |
| 43 | TYR | O | 22.32 | 67.34 | 1.24 | 9.56 |
| 44 | VAL | N | 20.14 | 67.28 | 1.76 | 9.59 |
| 44 | VAL | CA | 20.16 | 65.85 | 1.99 | 9.63 |
| 44 | VAL | CB | 18.82 | 65.36 | 2.58 | 9.59 |
| 44 | VAL | CG1 | 18.82 | 63.84 | 2.71 | 9.75 |
| 44 | VAL | CG2 | 18.59 | 66.00 | 3.95 | 9.71 |
| 44 | VAL | C | 20.49 | 65.11 | 0.70 | 9.61 |
| 44 | VAL | O | 21.28 | 64.16 | 0.71 | 9.46 |
| 45 | ARG | N | 19.91 | 65.53 | −0.42 | 9.76 |
| 45 | ARG | CA | 20.20 | 64.88 | −1.69 | 10.00 |
| 45 | ARG | CB | 19.36 | 65.49 | −2.83 | 10.27 |
| 45 | ARG | CG | 17.88 | 65.19 | −2.73 | 10.61 |
| 45 | ARG | CD | 17.15 | 65.58 | −4.00 | 11.03 |
| 45 | ARG | NE | 15.74 | 65.19 | −3.95 | 11.30 |
| 45 | ARG | CZ | 14.95 | 65.09 | −5.01 | 11.53 |
| 45 | ARG | NH1 | 15.41 | 65.34 | −6.23 | 11.82 |
| 45 | ARG | NH2 | 13.68 | 64.72 | −4.85 | 11.71 |
| 45 | ARG | C | 21.68 | 65.03 | −2.03 | 10.07 |
| 45 | ARG | O | 22.32 | 64.07 | −2.48 | 9.97 |
| 46 | ARG | N | 22.23 | 66.22 | −1.83 | 10.29 |
| 46 | ARG | CA | 23.63 | 66.46 | −2.12 | 10.60 |
| 46 | ARG | CB | 23.99 | 67.93 | −1.90 | 10.96 |
| 46 | ARG | CG | 25.48 | 68.22 | −2.04 | 11.57 |
| 46 | ARG | CD | 25.76 | 69.71 | −2.17 | 12.15 |
| 46 | ARG | NE | 25.54 | 70.46 | −0.93 | 12.71 |
| 46 | ARG | CZ | 26.38 | 70.48 | 0.10 | 12.98 |
| 46 | ARG | NH1 | 27.50 | 69.79 | 0.07 | 13.26 |
| 46 | ARG | NH2 | 26.09 | 71.22 | 1.16 | 13.28 |
| 46 | ARG | C | 24.55 | 65.58 | −1.27 | 10.61 |
| 46 | ARG | O | 25.47 | 64.96 | −1.80 | 10.50 |
| 47 | ARG | N | 24.30 | 65.53 | 0.03 | 10.66 |
| 47 | ARG | CA | 25.13 | 64.72 | 0.91 | 10.86 |
| 47 | ARG | CB | 24.72 | 64.91 | 2.38 | 11.39 |
| 47 | ARG | CG | 25.00 | 66.30 | 2.97 | 12.17 |
| 47 | ARG | CD | 26.50 | 66.61 | 3.08 | 12.90 |
| 47 | ARG | NE | 27.24 | 65.66 | 3.91 | 13.49 |
| 47 | ARG | CZ | 27.12 | 65.54 | 5.23 | 13.94 |
| 47 | ARG | NH1 | 26.29 | 66.32 | 5.92 | 14.29 |
| 47 | ARG | NH2 | 27.85 | 64.64 | 5.87 | 14.19 |
| 47 | ARG | C | 25.04 | 63.24 | 0.53 | 10.71 |
| 47 | ARG | O | 26.05 | 62.53 | 0.54 | 10.57 |
| 48 | CYS | N | 23.84 | 62.77 | 0.17 | 10.69 |
| 48 | CYS | CA | 23.67 | 61.38 | −0.23 | 10.74 |
| 48 | CYS | CB | 22.20 | 61.08 | −0.52 | 10.75 |
| 48 | CYS | SG | 21.20 | 60.81 | 0.96 | 10.77 |
| 48 | CYS | C | 24.50 | 61.08 | −1.47 | 10.86 |
| 48 | CYS | O | 25.15 | 60.04 | −1.56 | 10.67 |
| 49 | LYS | N | 24.49 | 61.99 | −2.43 | 11.07 |
| 49 | LYS | CA | 25.27 | 61.79 | −3.65 | 11.50 |
| 49 | LYS | CB | 24.86 | 62.81 | −4.72 | 11.73 |
| 49 | LYS | CG | 23.48 | 62.54 | −5.30 | 12.10 |
| 49 | LYS | CD | 23.12 | 63.53 | −6.40 | 12.58 |
| 49 | LYS | CE | 21.78 | 63.20 | −7.04 | 13.00 |
| 49 | LYS | NZ | 21.83 | 61.95 | −7.85 | 13.41 |
| 49 | LYS | C | 26.77 | 61.87 | −3.37 | 11.63 |
| 49 | LYS | O | 27.56 | 61.15 | −3.99 | 11.76 |
| 50 | GLU | N | 27.18 | 62.73 | −2.44 | 11.82 |
| 50 | GLU | CA | 28.59 | 62.87 | −2.12 | 12.09 |
| 50 | GLU | CB | 28.83 | 64.11 | −1.25 | 12.47 |
| 50 | GLU | CG | 28.48 | 65.42 | −1.94 | 13.13 |
| 50 | GLU | CD | 28.69 | 66.63 | −1.05 | 13.46 |
| 50 | GLU | OE1 | 28.64 | 66.48 | 0.19 | 13.90 |
| 50 | GLU | OE2 | 28.89 | 67.74 | −1.59 | 13.80 |
| 50 | GLU | C | 29.16 | 61.64 | −1.40 | 12.04 |
| 50 | GLU | O | 30.32 | 61.27 | −1.63 | 12.06 |
| 51 | GLU | N | 28.36 | 61.00 | −0.55 | 11.99 |
| 51 | GLU | CA | 28.83 | 59.85 | 0.21 | 11.97 |
| 51 | GLU | CB | 28.47 | 60.03 | 1.69 | 12.48 |
| 51 | GLU | CG | 28.96 | 61.34 | 2.29 | 13.24 |
| 51 | GLU | CD | 30.45 | 61.57 | 2.07 | 13.67 |
| 51 | GLU | OE1 | 31.21 | 60.59 | 2.13 | 14.03 |
| 51 | GLU | OE2 | 30.85 | 62.73 | 1.84 | 14.15 |
| 51 | GLU | C | 28.32 | 58.49 | −0.27 | 11.64 |
| 51 | GLU | O | 28.65 | 57.45 | 0.30 | 11.54 |
| 52 | ASN | N | 27.51 | 58.51 | −1.32 | 11.27 |
| 52 | ASN | CA | 26.94 | 57.30 | −1.92 | 10.99 |
| 52 | ASN | CB | 28.05 | 56.36 | −2.40 | 11.09 |
| 52 | ASN | CG | 27.73 | 55.74 | −3.76 | 11.19 |
| 52 | ASN | OD1 | 27.39 | 56.46 | −4.70 | 11.28 |
| 52 | ASN | ND2 | 27.84 | 54.42 | −3.86 | 11.27 |
| 52 | ASN | C | 25.94 | 56.54 | −1.04 | 10.74 |
| 52 | ASN | O | 26.06 | 55.33 | −0.82 | 10.77 |
| 53 | PHE | N | 24.94 | 57.28 | −0.56 | 10.56 |
| 53 | PHE | CA | 23.86 | 56.74 | 0.25 | 10.50 |
| 53 | PHE | CB | 23.70 | 57.51 | 1.56 | 10.73 |
| 53 | PHE | CG | 24.59 | 57.05 | 2.66 | 11.04 |
| 53 | PHE | CD1 | 25.95 | 57.34 | 2.65 | 11.25 |
| 53 | PHE | CD2 | 24.06 | 56.34 | 3.73 | 11.30 |
| 53 | PHE | CE1 | 26.77 | 56.93 | 3.69 | 11.50 |
| 53 | PHE | CE2 | 24.88 | 55.93 | 4.78 | 11.58 |
| 53 | PHE | CZ | 26.23 | 56.22 | 4.76 | 11.60 |
| 53 | PHE | C | 22.56 | 56.91 | −0.53 | 10.37 |
| 53 | PHE | O | 22.39 | 57.90 | −1.24 | 10.34 |
| 54 | LEU | N | 21.65 | 55.96 | −0.37 | 10.33 |
| 54 | LEU | CA | 20.36 | 56.06 | −1.03 | 10.31 |
| 54 | LEU | CB | 19.82 | 54.68 | −1.42 | 10.60 |
| 54 | LEU | CG | 20.31 | 54.01 | −2.70 | 10.82 |
| 54 | LEU | CD1 | 19.74 | 52.60 | −2.77 | 11.07 |
| 54 | LEU | CD2 | 19.89 | 54.82 | −3.91 | 11.04 |
| 54 | LEU | C | 19.34 | 56.70 | −0.10 | 10.18 |
| 54 | LEU | O | 19.30 | 56.40 | 1.09 | 9.97 |
| 55 | PRO | N | 18.52 | 57.61 | −0.64 | 10.13 |
| 55 | PRO | CD | 18.69 | 58.27 | −1.95 | 10.28 |
| 55 | PRO | CA | 17.47 | 58.27 | 0.14 | 10.05 |
| 55 | PRO | CB | 17.23 | 59.56 | −0.63 | 10.24 |
| 55 | PRO | CG | 17.47 | 59.14 | −2.05 | 10.41 |
| 55 | PRO | C | 16.32 | 57.26 | 0.03 | 9.92 |
| 55 | PRO | O | 15.52 | 57.32 | −0.91 | 9.80 |
| 56 | LEU | N | 16.27 | 56.33 | 0.97 | 9.87 |
| 56 | LEU | CA | 15.30 | 55.25 | 0.95 | 9.88 |
| 56 | LEU | CB | 15.59 | 54.28 | 2.10 | 9.94 |
| 56 | LEU | CG | 16.97 | 53.62 | 2.07 | 9.97 |
| 56 | LEU | CD1 | 17.14 | 52.71 | 3.27 | 10.21 |
| 56 | LEU | CD2 | 17.16 | 52.82 | 0.78 | 10.09 |
| 56 | LEU | C | 13.82 | 55.60 | 0.92 | 9.85 |
| 56 | LEU | O | 13.00 | 54.76 | 0.55 | 9.79 |
| 57 | GLN | N | 13.46 | 56.83 | 1.29 | 9.87 |
| 57 | GLN | CA | 12.05 | 57.21 | 1.26 | 10.08 |
| 57 | GLN | CB | 11.81 | 58.48 | 2.06 | 10.49 |
| 57 | GLN | CG | 12.02 | 58.31 | 3.55 | 11.18 |
| 57 | GLN | CD | 11.77 | 59.58 | 4.33 | 11.73 |
| 57 | GLN | OE1 | 12.10 | 59.65 | 5.51 | 12.28 |
| 57 | GLN | NE2 | 11.19 | 60.58 | 3.67 | 12.12 |
| 57 | GLN | C | 11.54 | 57.41 | −0.16 | 10.00 |
| 57 | GLN | O | 10.33 | 57.31 | −0.39 | 9.89 |
| 58 | ILE | N | 12.42 | 57.71 | −1.10 | 9.98 |
| 58 | ILE | CA | 11.97 | 57.92 | −2.47 | 10.12 |
| 58 | ILE | CB | 13.07 | 58.56 | −3.36 | 10.15 |
| 58 | ILE | CG2 | 12.52 | 58.81 | −4.76 | 10.27 |
| 58 | ILE | CG1 | 13.54 | 59.87 | −2.73 | 10.20 |
| 58 | ILE | CD1 | 14.57 | 60.64 | −3.55 | 10.31 |
| 58 | ILE | C | 11.52 | 56.60 | −3.08 | 10.17 |
| 58 | ILE | O | 12.32 | 55.70 | −3.33 | 10.24 |
| 59 | GLY | N | 10.21 | 56.49 | −3.29 | 10.21 |
| 59 | GLY | CA | 9.65 | 55.27 | −3.85 | 10.33 |
| 59 | GLY | C | 8.79 | 54.49 | −2.88 | 10.34 |
| 59 | GLY | O | 8.21 | 53.47 | −3.24 | 10.41 |
| 60 | VAL | N | 8.70 | 54.95 | −1.63 | 10.31 |
| 60 | VAL | CA | 7.87 | 54.24 | −0.65 | 10.38 |
| 60 | VAL | CB | 8.01 | 54.88 | 0.76 | 10.53 |
| 60 | VAL | CG1 | 6.93 | 54.35 | 1.70 | 10.74 |
| 60 | VAL | CG2 | 9.40 | 54.58 | 1.34 | 10.63 |
| 60 | VAL | C | 6.40 | 54.24 | −1.08 | 10.30 |
| 60 | VAL | O | 5.88 | 55.28 | −1.50 | 10.21 |
| 61 | ASP | N | 5.75 | 53.08 | −1.00 | 10.34 |
| 61 | ASP | CA | 4.34 | 52.93 | −1.38 | 10.43 |
| 61 | ASP | CB | 3.76 | 51.59 | −0.87 | 10.62 |
| 61 | ASP | CG | 4.19 | 50.39 | −1.69 | 10.82 |
| 61 | ASP | OD1 | 4.86 | 50.52 | −2.74 | 11.09 |
| 61 | ASP | OD2 | 3.83 | 49.27 | −1.25 | 11.02 |
| 61 | ASP | C | 3.43 | 54.01 | −0.80 | 10.45 |
| 61 | ASP | O | 3.47 | 54.30 | 0.40 | 10.44 |
| 62 | GLY | N | 2.57 | 54.56 | −1.66 | 10.46 |
| 62 | GLY | CA | 1.60 | 55.57 | −1.26 | 10.51 |
| 62 | GLY | C | 0.38 | 55.36 | −2.15 | 10.42 |
| 62 | GLY | O | 0.54 | 55.10 | −3.34 | 10.43 |
| 63 | ALA | N | −0.83 | 55.48 | −1.60 | 10.45 |
| 63 | ALA | CA | −2.05 | 55.25 | −2.37 | 10.51 |
| 63 | ALA | CB | −3.25 | 55.23 | −1.43 | 10.64 |
| 63 | ALA | C | −2.31 | 56.22 | −3.52 | 10.54 |
| 63 | ALA | O | −2.81 | 55.81 | −4.57 | 10.52 |
| 64 | MET | N | −2.00 | 57.49 | −3.32 | 10.56 |
| 64 | MET | CA | −2.21 | 58.48 | −4.37 | 10.71 |
| 64 | MET | CB | −2.64 | 59.82 | −3.76 | 11.02 |
| 64 | MET | CG | −3.92 | 59.74 | −2.96 | 11.50 |
| 64 | MET | SD | −4.57 | 61.33 | −2.41 | 11.77 |
| 64 | MET | CE | −5.40 | 61.88 | −3.91 | 12.10 |
| 64 | MET | C | −0.95 | 58.68 | −5.20 | 10.59 |
| 64 | MET | O | −1.02 | 59.03 | −6.38 | 10.57 |
| 65 | MET | N | 0.19 | 58.44 | −4.57 | 10.53 |
| 65 | MET | CA | 1.49 | 58.59 | −5.23 | 10.52 |
| 65 | MET | CB | 1.76 | 60.08 | −5.47 | 10.87 |
| 65 | MET | CG | 1.84 | 60.90 | −4.19 | 11.38 |
| 65 | MET | SD | 1.72 | 62.69 | −4.41 | 12.03 |
| 65 | MET | CE | 3.35 | 63.06 | −5.08 | 12.18 |
| 65 | MET | C | 2.54 | 58.03 | −4.29 | 10.27 |
| 65 | MET | O | 2.36 | 58.03 | −3.07 | 10.10 |
| 66 | ASP | N | 3.64 | 57.52 | −4.83 | 10.07 |
| 66 | ASP | CA | 4.69 | 57.01 | −3.97 | 9.94 |
| 66 | ASP | CB | 5.67 | 56.13 | −4.75 | 10.17 |
| 66 | ASP | CG | 5.09 | 54.76 | −5.06 | 10.26 |
| 66 | ASP | OD1 | 4.12 | 54.35 | −4.37 | 10.27 |
| 66 | ASP | OD2 | 5.61 | 54.09 | −5.97 | 10.50 |
| 66 | ASP | C | 5.40 | 58.23 | −3.38 | 9.79 |
| 66 | ASP | O | 5.31 | 59.33 | −3.94 | 9.84 |
| 67 | TYR | N | 6.09 | 58.06 | −2.26 | 9.61 |
| 67 | TYR | CA | 6.76 | 59.21 | −1.65 | 9.55 |
| 67 | TYR | CB | 7.32 | 58.85 | −0.28 | 9.34 |
| 67 | TYR | CG | 7.58 | 60.10 | 0.52 | 9.11 |
| 67 | TYR | CD1 | 6.53 | 60.85 | 1.05 | 9.14 |
| 67 | TYR | CE1 | 6.75 | 62.08 | 1.66 | 8.99 |
| 67 | TYR | CD2 | 8.87 | 60.61 | 0.64 | 8.95 |
| 67 | TYR | CE2 | 9.10 | 61.84 | 1.25 | 8.82 |
| 67 | TYR | CZ | 8.03 | 62.57 | 1.75 | 8.88 |
| 67 | TYR | OH | 8.23 | 63.82 | 2.29 | 8.74 |
| 67 | TYR | C | 7.88 | 59.68 | −2.58 | 9.66 |
| 67 | TYR | O | 8.75 | 58.90 | −2.96 | 9.69 |
| 68 | PRO | N | 7.90 | 60.97 | −2.94 | 9.80 |
| 68 | PRO | CD | 6.88 | 62.01 | −2.73 | 9.99 |
| 68 | PRO | CA | 8.94 | 61.46 | −3.85 | 9.89 |
| 68 | PRO | CB | 8.22 | 62.54 | −4.67 | 10.14 |
| 68 | PRO | CG | 6.80 | 62.63 | −4.09 | 10.22 |
| 68 | PRO | C | 10.23 | 62.03 | −3.31 | 9.76 |
| 68 | PRO | O | 11.17 | 62.24 | −4.08 | 9.83 |
| 69 | TYR | N | 10.31 | 62.25 | −2.00 | 9.61 |
| 69 | TYR | CA | 11.47 | 62.92 | −1.45 | 9.48 |
| 69 | TYR | CB | 11.00 | 64.23 | −0.82 | 9.82 |
| 69 | TYR | CG | 10.10 | 65.03 | −1.75 | 10.26 |
| 69 | TYR | CD1 | 10.61 | 65.65 | −2.88 | 10.51 |
| 69 | TYR | CE1 | 9.79 | 66.38 | −3.74 | 10.85 |
| 69 | TYR | CD2 | 8.72 | 65.14 | −1.50 | 10.51 |
| 69 | TYR | CE2 | 7.89 | 65.86 | −2.36 | 10.87 |
| 69 | TYR | CZ | 8.43 | 66.47 | −3.48 | 10.98 |
| 69 | TYR | OH | 7.62 | 67.19 | −4.33 | 11.40 |
| 69 | TYR | C | 12.35 | 62.17 | −0.46 | 9.26 |
| 69 | TYR | O | 11.93 | 61.20 | 0.17 | 9.06 |
| 70 | ALA | N | 13.58 | 62.65 | −0.32 | 9.11 |
| 70 | ALA | CA | 14.54 | 62.07 | 0.60 | 9.08 |
| 70 | ALA | CB | 15.92 | 62.65 | 0.35 | 9.09 |
| 70 | ALA | C | 14.10 | 62.38 | 2.04 | 9.03 |
| 70 | ALA | O | 14.30 | 61.57 | 2.95 | 9.03 |
| 71 | THR | N | 13.50 | 63.55 | 2.21 | 8.94 |
| 71 | THR | CA | 13.06 | 64.00 | 3.52 | 9.03 |
| 71 | THR | CB | 13.70 | 65.37 | 3.90 | 9.02 |
| 71 | THR | OG1 | 13.14 | 66.40 | 3.06 | 9.06 |
| 71 | THR | CG2 | 15.20 | 65.34 | 3.74 | 9.16 |
| 71 | THR | C | 11.57 | 64.23 | 3.65 | 8.95 |
| 71 | THR | O | 10.84 | 64.28 | 2.66 | 8.86 |
| 72 | CYS | N | 11.16 | 64.37 | 4.90 | 9.13 |
| 72 | CYS | CA | 9.80 | 64.74 | 5.26 | 9.21 |
| 72 | CYS | CB | 9.12 | 63.72 | 6.16 | 9.42 |
| 72 | CYS | SG | 8.58 | 62.24 | 5.31 | 9.76 |
| 72 | CYS | C | 10.08 | 66.02 | 6.05 | 9.17 |
| 72 | CYS | O | 10.90 | 66.02 | 6.97 | 9.29 |
| 73 | CYS | N | 9.43 | 67.10 | 5.65 | 9.11 |
| 73 | CYS | CA | 9.58 | 68.39 | 6.31 | 9.17 |
| 73 | CYS | CB | 10.12 | 69.44 | 5.33 | 9.40 |
| 73 | CYS | SG | 11.86 | 69.23 | 4.89 | 9.88 |
| 73 | CYS | C | 8.19 | 68.78 | 6.79 | 9.09 |
| 73 | CYS | O | 7.33 | 69.15 | 5.99 | 9.24 |
| 74 | SER | N | 7.98 | 68.68 | 8.10 | 9.04 |
| 74 | SER | CA | 6.69 | 68.98 | 8.70 | 9.04 |
| 74 | SER | CB | 6.25 | 67.79 | 9.54 | 8.93 |
| 74 | SER | OG | 6.17 | 66.62 | 8.72 | 8.79 |
| 74 | SER | C | 6.75 | 70.25 | 9.52 | 9.17 |
| 74 | SER | O | 7.42 | 70.33 | 10.56 | 9.04 |
| 75 | LEU | N | 6.03 | 71.25 | 9.03 | 9.46 |
| 75 | LEU | CA | 5.98 | 72.58 | 9.61 | 9.82 |
| 75 | LEU | CB | 5.91 | 73.58 | 8.46 | 10.54 |
| 75 | LEU | CG | 5.84 | 75.10 | 8.64 | 11.18 |
| 75 | LEU | CD1 | 7.15 | 75.60 | 9.21 | 11.54 |
| 75 | LEU | CD2 | 5.56 | 75.75 | 7.29 | 11.53 |
| 75 | LEU | C | 4.83 | 72.84 | 10.57 | 9.75 |
| 75 | LEU | O | 3.68 | 72.54 | 10.26 | 9.68 |
| 76 | ASN | N | 5.15 | 73.39 | 11.73 | 9.69 |
| 76 | ASN | CA | 4.17 | 73.78 | 12.73 | 9.64 |
| 76 | ASN | CB | 3.58 | 75.13 | 12.30 | 9.71 |
| 76 | ASN | CG | 4.67 | 76.19 | 12.15 | 9.72 |
| 76 | ASN | OD1 | 5.59 | 76.24 | 12.96 | 9.81 |
| 76 | ASN | ND2 | 4.57 | 77.03 | 11.12 | 9.97 |
| 76 | ASN | C | 3.08 | 72.77 | 13.09 | 9.58 |
| 76 | ASN | O | 3.35 | 71.84 | 13.85 | 9.51 |
| 77 | ASP | N | 1.86 | 72.94 | 12.57 | 9.59 |
| 77 | ASP | CA | 0.77 | 72.01 | 12.88 | 9.66 |
| 77 | ASP | CB | −0.60 | 72.68 | 12.70 | 10.11 |
| 77 | ASP | CG | −0.85 | 73.18 | 11.29 | 10.40 |
| 77 | ASP | OD1 | 0.12 | 73.56 | 10.61 | 11.01 |
| 77 | ASP | OD2 | −2.03 | 73.21 | 10.87 | 10.73 |
| 77 | ASP | C | 0.85 | 70.70 | 12.09 | 9.48 |
| 77 | ASP | O | 0.04 | 69.80 | 12.30 | 9.48 |
| 78 | GLU | N | 1.81 | 70.60 | 11.18 | 9.36 |
| 78 | GLU | CA | 2.01 | 69.37 | 10.43 | 9.23 |
| 78 | GLU | CB | 2.86 | 69.62 | 9.18 | 9.34 |
| 78 | GLU | CG | 2.16 | 70.54 | 8.19 | 9.61 |
| 78 | GLU | CD | 2.98 | 70.88 | 6.96 | 9.71 |
| 78 | GLU | OE1 | 4.22 | 70.71 | 6.99 | 9.73 |
| 78 | GLU | OE2 | 2.38 | 71.34 | 5.97 | 10.16 |
| 78 | GLU | C | 2.71 | 68.42 | 11.39 | 9.16 |
| 78 | GLU | O | 3.72 | 68.77 | 12.01 | 9.06 |
| 79 | VAL | N | 2.16 | 67.22 | 11.52 | 9.06 |
| 79 | VAL | CA | 2.67 | 66.22 | 12.44 | 9.09 |
| 79 | VAL | CB | 1.51 | 65.28 | 12.86 | 9.20 |
| 79 | VAL | CG1 | 2.02 | 64.16 | 13.74 | 9.36 |
| 79 | VAL | CG2 | 0.44 | 66.08 | 13.57 | 9.32 |
| 79 | VAL | C | 3.81 | 65.39 | 11.88 | 9.04 |
| 79 | VAL | O | 4.84 | 65.20 | 12.54 | 8.97 |
| 80 | ALA | N | 3.63 | 64.89 | 10.67 | 9.09 |
| 80 | ALA | CA | 4.63 | 64.05 | 10.03 | 9.22 |
| 80 | ALA | CB | 4.72 | 62.71 | 10.77 | 9.33 |
| 80 | ALA | C | 4.26 | 63.80 | 8.58 | 9.33 |
| 80 | ALA | O | 3.14 | 64.09 | 8.15 | 9.16 |
| 81 | HIS | N | 5.23 | 63.28 | 7.83 | 9.58 |
| 81 | HIS | CA | 5.06 | 62.91 | 6.43 | 9.91 |
| 81 | HIS | CB | 3.99 | 61.81 | 6.34 | 10.63 |
| 81 | HIS | CG | 4.28 | 60.61 | 7.18 | 11.50 |
| 81 | HIS | CD2 | 5.40 | 60.21 | 7.83 | 11.88 |
| 81 | HIS | ND1 | 3.33 | 59.65 | 7.44 | 12.00 |
| 81 | HIS | CE1 | 3.85 | 58.70 | 8.20 | 12.05 |
| 81 | HIS | NE2 | 5.11 | 59.02 | 8.45 | 12.09 |
| 81 | HIS | C | 4.74 | 64.01 | 5.42 | 9.68 |
| 81 | HIS | O | 4.28 | 63.72 | 4.32 | 9.75 |
| 82 | ALA | N | 4.98 | 65.26 | 5.79 | 9.53 |
| 82 | ALA | CA | 4.74 | 66.37 | 4.88 | 9.49 |
| 82 | ALA | CB | 4.46 | 67.65 | 5.66 | 9.49 |
| 82 | ALA | C | 5.97 | 66.55 | 3.98 | 9.47 |
| 82 | ALA | O | 7.09 | 66.16 | 4.34 | 9.34 |
| 83 | PHE | N | 5.76 | 67.15 | 2.81 | 9.71 |
| 83 | PHE | CA | 6.83 | 67.33 | 1.83 | 9.93 |
| 83 | PHE | CB | 6.29 | 67.46 | 0.40 | 10.21 |
| 83 | PHE | CG | 5.37 | 66.38 | −0.08 | 10.50 |
| 83 | PHE | CD1 | 5.48 | 65.06 | 0.35 | 10.63 |
| 83 | PHE | CD2 | 4.46 | 66.68 | −1.09 | 10.79 |
| 83 | PHE | CE1 | 4.70 | 64.06 | −0.23 | 10.81 |
| 83 | PHE | CE2 | 3.67 | 65.70 | −1.67 | 10.97 |
| 83 | PHE | CZ | 3.79 | 64.39 | −1.25 | 10.99 |
| 83 | PHE | C | 7.71 | 68.56 | 1.94 | 9.98 |
| 83 | PHE | O | 7.28 | 69.62 | 2.39 | 9.86 |
| 84 | PRO | N | 8.97 | 68.42 | 1.50 | 10.05 |
| 84 | PRO | CD | 9.76 | 67.21 | 1.25 | 10.06 |
| 84 | PRO | CA | 9.82 | 69.61 | 1.53 | 10.16 |
| 84 | PRO | CB | 11.20 | 69.06 | 1.16 | 10.08 |
| 84 | PRO | CG | 10.90 | 67.75 | 0.45 | 10.04 |
| 84 | PRO | C | 9.16 | 70.36 | 0.36 | 10.30 |
| 84 | PRO | O | 8.64 | 69.72 | −0.57 | 10.33 |
| 85 | ARG | N | 9.14 | 71.69 | 0.39 | 10.52 |
| 85 | ARG | CA | 8.48 | 72.45 | −0.66 | 10.92 |
| 85 | ARG | CB | 6.98 | 72.54 | −0.36 | 11.19 |
| 85 | ARG | CG | 6.66 | 73.55 | 0.75 | 11.49 |
| 85 | ARG | CD | 5.45 | 73.17 | 1.59 | 11.58 |
| 85 | ARG | NE | 5.74 | 72.06 | 2.50 | 11.59 |
| 85 | ARG | CZ | 5.09 | 71.82 | 3.63 | 11.44 |
| 85 | ARG | NH1 | 4.09 | 72.61 | 4.00 | 11.59 |
| 85 | ARG | NH2 | 5.43 | 70.79 | 4.39 | 11.31 |
| 85 | ARG | C | 9.07 | 73.85 | −0.69 | 11.03 |
| 85 | ARG | O | 9.80 | 74.24 | 0.21 | 10.93 |
| 86 | HIS | N | 8.75 | 74.61 | −1.74 | 11.28 |
| 86 | HIS | CA | 9.23 | 75.98 | −1.82 | 11.59 |
| 86 | HIS | CB | 9.08 | 76.52 | −3.24 | 12.02 |
| 86 | HIS | CG | 10.07 | 75.94 | −4.20 | 12.51 |
| 86 | HIS | CD2 | 9.90 | 75.19 | −5.31 | 12.80 |
| 86 | HIS | ND1 | 11.43 | 76.08 | −4.03 | 12.66 |
| 86 | HIS | CE1 | 12.06 | 75.43 | −5.00 | 12.95 |
| 86 | HIS | NE2 | 11.15 | 74.88 | −5.79 | 13.10 |
| 86 | HIS | C | 8.41 | 76.80 | −0.84 | 11.56 |
| 86 | HIS | O | 7.21 | 76.98 | −1.00 | 11.75 |
| 87 | TYR | N | 9.10 | 77.27 | 0.19 | 11.47 |
| 87 | TYR | CA | 8.48 | 78.06 | 1.25 | 11.44 |
| 87 | TYR | CB | 7.75 | 77.13 | 2.23 | 11.38 |
| 87 | TYR | CG | 7.40 | 77.79 | 3.54 | 11.30 |
| 87 | TYR | CD1 | 6.40 | 78.76 | 3.62 | 11.36 |
| 87 | TYR | CE1 | 6.11 | 79.40 | 4.82 | 11.32 |
| 87 | TYR | CD2 | 8.10 | 77.48 | 4.71 | 11.20 |
| 87 | TYR | CE2 | 7.82 | 78.11 | 5.91 | 11.20 |
| 87 | TYR | CZ | 6.83 | 79.08 | 5.96 | 11.21 |
| 87 | TYR | OH | 6.55 | 79.72 | 7.14 | 11.19 |
| 87 | TYR | C | 9.61 | 78.77 | 1.99 | 11.41 |
| 87 | TYR | O | 10.57 | 78.13 | 2.41 | 11.37 |
| 88 | ILE | N | 9.51 | 80.08 | 2.12 | 11.50 |
| 88 | ILE | CA | 10.54 | 80.83 | 2.81 | 11.59 |
| 88 | ILE | CB | 10.53 | 82.31 | 2.38 | 11.84 |
| 88 | ILE | CG2 | 11.59 | 83.09 | 3.14 | 11.91 |
| 88 | ILE | CG1 | 10.77 | 82.40 | 0.86 | 12.04 |
| 88 | ILE | CD1 | 12.04 | 81.72 | 0.38 | 12.32 |
| 88 | ILE | C | 10.31 | 80.71 | 4.32 | 11.50 |
| 88 | ILE | O | 9.31 | 81.20 | 4.85 | 11.40 |
| 89 | LEU | N | 11.24 | 80.05 | 4.99 | 11.42 |
| 89 | LEU | CA | 11.14 | 79.84 | 6.43 | 11.43 |
| 89 | LEU | CB | 12.36 | 79.06 | 6.93 | 11.43 |
| 89 | LEU | CG | 12.48 | 77.63 | 6.37 | 11.41 |
| 89 | LEU | CD1 | 13.85 | 77.07 | 6.70 | 11.54 |
| 89 | LEU | CD2 | 11.38 | 76.75 | 6.95 | 11.43 |
| 89 | LEU | C | 11.01 | 81.16 | 7.19 | 11.50 |
| 89 | LEU | O | 11.72 | 82.13 | 6.90 | 11.51 |
| 90 | LYS | N | 10.10 | 81.19 | 8.15 | 11.67 |
| 90 | LYS | CA | 9.85 | 82.38 | 8.95 | 11.94 |
| 90 | LYS | CB | 8.35 | 82.62 | 9.09 | 12.29 |
| 90 | LYS | CG | 7.59 | 82.83 | 7.79 | 12.79 |
| 90 | LYS | CD | 6.10 | 83.01 | 8.08 | 13.24 |
| 90 | LYS | CE | 5.28 | 83.11 | 6.81 | 13.63 |
| 90 | LYS | NZ | 5.69 | 84.26 | 5.97 | 13.91 |
| 90 | LYS | C | 10.43 | 82.25 | 10.35 | 11.89 |
| 90 | LYS | O | 10.47 | 81.16 | 10.93 | 11.78 |
| 91 | ASP | N | 10.87 | 83.38 | 10.90 | 11.98 |
| 91 | ASP | CA | 11.40 | 83.41 | 12.25 | 12.05 |
| 91 | ASP | CB | 11.85 | 84.83 | 12.60 | 12.62 |
| 91 | ASP | CG | 12.77 | 84.87 | 13.80 | 13.09 |
| 91 | ASP | OD1 | 12.41 | 84.28 | 14.84 | 13.56 |
| 91 | ASP | OD2 | 13.84 | 85.49 | 13.70 | 13.74 |
| 91 | ASP | C | 10.21 | 83.00 | 13.13 | 11.83 |
| 91 | ASP | O | 9.16 | 83.64 | 13.10 | 12.01 |
| 92 | GLY | N | 10.38 | 81.93 | 13.90 | 11.49 |
| 92 | GLY | CA | 9.30 | 81.47 | 14.75 | 11.11 |
| 92 | GLY | C | 8.72 | 80.13 | 14.33 | 10.82 |
| 92 | GLY | O | 8.02 | 79.49 | 15.11 | 10.82 |
| 93 | ASP | N | 9.02 | 79.71 | 13.10 | 10.51 |
| 93 | ASP | CA | 8.53 | 78.43 | 12.59 | 10.25 |
| 93 | ASP | CB | 8.86 | 78.25 | 11.10 | 10.19 |
| 93 | ASP | CG | 7.87 | 78.94 | 10.18 | 10.10 |
| 93 | ASP | OD1 | 6.71 | 79.20 | 10.59 | 10.16 |
| 93 | ASP | OD2 | 8.25 | 79.19 | 9.01 | 10.09 |
| 93 | ASP | C | 9.18 | 77.27 | 13.34 | 10.11 |
| 93 | ASP | O | 10.29 | 77.39 | 13.88 | 10.12 |
| 94 | LEU | N | 8.48 | 76.14 | 13.34 | 9.97 |
| 94 | LEU | CA | 8.96 | 74.90 | 13.95 | 9.92 |
| 94 | LEU | CB | 8.00 | 74.43 | 15.04 | 10.10 |
| 94 | LEU | CG | 8.23 | 73.02 | 15.58 | 10.27 |
| 94 | LEU | CD1 | 9.63 | 72.90 | 16.18 | 10.53 |
| 94 | LEU | CD2 | 7.17 | 72.68 | 16.61 | 10.60 |
| 94 | LEU | C | 8.97 | 73.89 | 12.80 | 9.74 |
| 94 | LEU | O | 7.94 | 73.61 | 12.20 | 9.74 |
| 95 | LEU | N | 10.15 | 73.35 | 12.51 | 9.64 |
| 95 | LEU | CA | 10.29 | 72.41 | 11.40 | 9.52 |
| 95 | LEU | CB | 11.20 | 73.01 | 10.33 | 10.05 |
| 95 | LEU | CG | 11.42 | 72.18 | 9.06 | 10.56 |
| 95 | LEU | CD1 | 10.11 | 72.06 | 8.30 | 11.11 |
| 95 | LEU | CD2 | 12.47 | 72.84 | 8.18 | 10.82 |
| 95 | LEU | C | 10.85 | 71.06 | 11.85 | 9.20 |
| 95 | LEU | O | 11.94 | 71.00 | 12.42 | 9.16 |
| 96 | LYS | N | 10.11 | 69.99 | 11.58 | 8.91 |
| 96 | LYS | CA | 10.55 | 68.64 | 11.92 | 8.77 |
| 96 | LYS | CB | 9.40 | 67.81 | 12.51 | 8.82 |
| 96 | LYS | CG | 8.94 | 68.25 | 13.91 | 9.02 |
| 96 | LYS | CD | 7.97 | 69.42 | 13.88 | 9.17 |
| 96 | LYS | CE | 6.61 | 69.01 | 13.35 | 9.28 |
| 96 | LYS | NZ | 5.68 | 70.18 | 13.26 | 9.36 |
| 96 | LYS | C | 11.05 | 67.99 | 10.63 | 8.67 |
| 96 | LYS | O | 10.30 | 67.86 | 9.66 | 8.74 |
| 97 | VAL | N | 12.32 | 67.59 | 10.61 | 8.56 |
| 97 | VAL | CA | 12.91 | 66.97 | 9.43 | 8.53 |
| 97 | VAL | CB | 14.20 | 67.70 | 8.99 | 8.64 |
| 97 | VAL | CG1 | 14.76 | 67.07 | 7.72 | 8.87 |
| 97 | VAL | CG2 | 13.92 | 69.17 | 8.77 | 8.84 |
| 97 | VAL | C | 13.24 | 65.51 | 9.71 | 8.36 |
| 97 | VAL | O | 14.03 | 65.20 | 10.61 | 8.30 |
| 98 | ASP | N | 12.61 | 64.61 | 8.96 | 8.34 |
| 98 | ASP | CA | 12.82 | 63.17 | 9.11 | 8.38 |
| 98 | ASP | CB | 11.49 | 62.50 | 9.48 | 8.42 |
| 98 | ASP | CG | 11.66 | 61.12 | 10.13 | 8.44 |
| 98 | ASP | OD1 | 12.77 | 60.54 | 10.09 | 8.33 |
| 98 | ASP | OD2 | 10.67 | 60.61 | 10.68 | 8.63 |
| 98 | ASP | C | 13.35 | 62.64 | 7.78 | 8.39 |
| 98 | ASP | O | 13.07 | 63.20 | 6.71 | 8.48 |
| 99 | MET | N | 14.12 | 61.56 | 7.85 | 8.34 |
| 99 | MET | CA | 14.70 | 60.94 | 6.67 | 8.48 |
| 99 | MET | CB | 15.90 | 61.76 | 6.16 | 8.66 |
| 99 | MET | CG | 17.11 | 61.71 | 7.08 | 8.98 |
| 99 | MET | SD | 18.35 | 62.96 | 6.71 | 9.33 |
| 99 | MET | CE | 17.62 | 64.39 | 7.51 | 9.50 |
| 99 | MET | C | 15.16 | 59.53 | 7.01 | 8.49 |
| 99 | MET | O | 15.39 | 59.21 | 8.17 | 8.45 |
| 100 | VAL | N | 15.27 | 58.69 | 5.98 | 8.46 |
| 100 | VAL | CA | 15.73 | 57.32 | 6.14 | 8.61 |
| 100 | VAL | CB | 14.60 | 56.29 | 5.93 | 8.70 |
| 100 | VAL | CG1 | 15.13 | 54.89 | 6.17 | 8.87 |
| 100 | VAL | CG2 | 13.44 | 56.59 | 6.88 | 8.82 |
| 100 | VAL | C | 16.80 | 57.15 | 5.06 | 8.73 |
| 100 | VAL | O | 16.54 | 57.35 | 3.87 | 8.71 |
| 101 | LEU | N | 18.01 | 56.79 | 5.49 | 8.92 |
| 101 | LEU | CA | 19.14 | 56.65 | 4.58 | 9.17 |
| 101 | LEU | CB | 20.20 | 57.71 | 4.92 | 9.45 |
| 101 | LEU | CG | 19.70 | 59.14 | 5.14 | 9.76 |
| 101 | LEU | CD1 | 20.85 | 60.01 | 5.59 | 10.04 |
| 101 | LEU | CD2 | 19.08 | 59.69 | 3.86 | 10.01 |
| 101 | LEU | C | 19.78 | 55.27 | 4.63 | 9.24 |
| 101 | LEU | O | 19.88 | 54.67 | 5.70 | 9.19 |
| 102 | GLY | N | 20.21 | 54.79 | 3.47 | 9.31 |
| 102 | GLY | CA | 20.85 | 53.49 | 3.39 | 9.54 |
| 102 | GLY | C | 22.20 | 53.57 | 2.70 | 9.75 |
| 102 | GLY | O | 22.31 | 54.08 | 1.59 | 9.71 |
| 103 | GLY | N | 23.23 | 53.06 | 3.37 | 9.94 |
| 103 | GLY | CA | 24.56 | 53.09 | 2.80 | 10.39 |
| 103 | GLY | C | 25.63 | 52.91 | 3.86 | 10.70 |
| 103 | GLY | O | 25.30 | 52.59 | 5.00 | 10.73 |
| 104 | PRO | N | 26.91 | 53.12 | 3.52 | 10.98 |
| 104 | PRO | CD | 28.04 | 53.01 | 4.45 | 11.14 |
| 104 | PRO | CA | 27.37 | 53.51 | 2.18 | 11.32 |
| 104 | PRO | CB | 28.77 | 54.02 | 2.45 | 11.31 |
| 104 | PRO | CG | 29.24 | 53.09 | 3.52 | 11.26 |
| 104 | PRO | C | 27.35 | 52.35 | 1.20 | 11.55 |
| 104 | PRO | O | 27.67 | 51.22 | 1.56 | 11.68 |
| 105 | ILE | N | 26.97 | 52.64 | −0.04 | 11.82 |
| 105 | ILE | CA | 26.92 | 51.62 | −1.07 | 12.14 |
| 105 | ILE | CB | 25.73 | 51.89 | −2.02 | 12.29 |
| 105 | ILE | CG2 | 25.76 | 50.94 | −3.21 | 12.38 |
| 105 | ILE | CG1 | 24.42 | 51.74 | −1.23 | 12.45 |
| 105 | ILE | CD1 | 23.23 | 52.38 | −1.90 | 12.56 |
| 105 | ILE | C | 28.23 | 51.62 | −1.86 | 12.23 |
| 105 | ILE | O | 28.84 | 52.67 | −2.05 | 12.08 |
| 106 | ALA | N | 28.65 | 50.44 | −2.30 | 12.53 |
| 106 | ALA | CA | 29.89 | 50.32 | −3.08 | 12.97 |
| 106 | ALA | CB | 30.15 | 48.86 | −3.42 | 13.00 |
| 106 | ALA | C | 29.76 | 51.15 | −4.35 | 13.34 |
| 106 | ALA | O | 28.85 | 50.94 | −5.15 | 13.16 |
| 107 | LYS | N | 30.69 | 52.08 | −4.52 | 13.94 |
| 107 | LYS | CA | 30.71 | 52.98 | −5.67 | 14.61 |
| 107 | LYS | CB | 31.97 | 53.85 | −5.62 | 15.02 |
| 107 | LYS | CG | 31.97 | 55.06 | −6.54 | 15.57 |
| 107 | LYS | CD | 31.02 | 56.14 | −6.04 | 16.00 |
| 107 | LYS | CE | 31.16 | 57.41 | −6.87 | 16.19 |
| 107 | LYS | NZ | 30.30 | 58.51 | −6.37 | 16.45 |
| 107 | LYS | C | 30.66 | 52.23 | −7.00 | 14.81 |
| 107 | LYS | O | 29.99 | 52.67 | −7.94 | 14.99 |
| 108 | SER | N | 31.36 | 51.11 | −7.08 | 14.96 |
| 108 | SER | CA | 31.40 | 50.32 | −8.31 | 15.12 |
| 108 | SER | CB | 32.61 | 49.40 | −8.30 | 15.29 |
| 108 | SER | OG | 32.67 | 48.64 | −7.11 | 15.62 |
| 108 | SER | C | 30.13 | 49.51 | −8.58 | 15.11 |
| 108 | SER | O | 29.97 | 48.95 | −9.66 | 15.15 |
| 109 | ASP | N | 29.23 | 49.45 | −7.60 | 15.11 |
| 109 | ASP | CA | 27.98 | 48.72 | −7.77 | 15.20 |
| 109 | ASP | CB | 27.60 | 47.96 | −6.50 | 15.13 |
| 109 | ASP | CG | 28.53 | 46.80 | −6.21 | 15.05 |
| 109 | ASP | OD1 | 29.28 | 46.39 | −7.13 | 14.76 |
| 109 | ASP | OD2 | 28.50 | 46.27 | −5.08 | 15.20 |
| 109 | ASP | C | 26.86 | 49.68 | −8.14 | 15.28 |
| 109 | ASP | O | 26.01 | 49.37 | −8.98 | 15.36 |
| 110 | LEU | N | 26.85 | 50.85 | −7.51 | 15.38 |
| 110 | LEU | CA | 25.83 | 51.85 | −7.77 | 15.44 |
| 110 | LEU | CB | 24.59 | 51.56 | −6.91 | 15.65 |
| 110 | LEU | CG | 23.30 | 52.32 | −7.23 | 15.83 |
| 110 | LEU | CD1 | 22.78 | 51.88 | −8.59 | 15.95 |
| 110 | LEU | CD2 | 22.26 | 52.05 | −6.16 | 15.98 |
| 110 | LEU | C | 26.36 | 53.24 | −7.45 | 15.37 |
| 110 | LEU | O | 26.68 | 53.53 | −6.30 | 15.41 |
| 111 | ASN | N | 26.47 | 54.09 | −8.47 | 15.23 |
| 111 | ASN | CA | 26.95 | 55.45 | −8.28 | 15.11 |
| 111 | ASN | CB | 27.72 | 55.94 | −9.51 | 15.42 |
| 111 | ASN | CG | 28.43 | 57.25 | −9.26 | 15.67 |
| 111 | ASN | OD1 | 27.93 | 58.11 | −8.54 | 15.83 |
| 111 | ASN | ND2 | 29.61 | 57.42 | −9.86 | 15.96 |
| 111 | ASN | C | 25.73 | 56.34 | −8.06 | 14.84 |
| 111 | ASN | O | 25.08 | 56.77 | −9.01 | 14.71 |
| 112 | VAL | N | 25.44 | 56.62 | −6.80 | 14.60 |
| 112 | VAL | CA | 24.29 | 57.44 | −6.44 | 14.38 |
| 112 | VAL | CB | 24.20 | 57.58 | −4.90 | 14.32 |
| 112 | VAL | CG1 | 23.01 | 58.44 | −4.52 | 14.25 |
| 112 | VAL | CG2 | 24.08 | 56.19 | −4.26 | 14.29 |
| 112 | VAL | C | 24.26 | 58.82 | −7.09 | 14.34 |
| 112 | VAL | O | 23.19 | 59.33 | −7.42 | 14.15 |
| 113 | SER | N | 25.43 | 59.42 | −7.31 | 14.39 |
| 113 | SER | CA | 25.47 | 60.74 | −7.93 | 14.57 |
| 113 | SER | CB | 26.90 | 61.30 | −7.91 | 14.67 |
| 113 | SER | OG | 27.75 | 60.59 | −8.78 | 14.92 |
| 113 | SER | C | 24.95 | 60.71 | −9.37 | 14.60 |
| 113 | SER | O | 24.57 | 61.74 | −9.92 | 14.74 |
| 114 | LYS | N | 24.94 | 59.53 | −9.98 | 14.56 |
| 114 | LYS | CA | 24.47 | 59.38 | −11.35 | 14.56 |
| 114 | LYS | CB | 25.45 | 58.55 | −12.17 | 14.85 |
| 114 | LYS | CG | 26.77 | 59.26 | −12.42 | 15.26 |
| 114 | LYS | CD | 27.71 | 58.38 | −13.22 | 15.56 |
| 114 | LYS | CE | 29.04 | 59.07 | −13.44 | 15.75 |
| 114 | LYS | NZ | 29.98 | 58.16 | −14.14 | 15.96 |
| 114 | LYS | C | 23.08 | 58.77 | −11.44 | 14.37 |
| 114 | LYS | O | 22.64 | 58.36 | −12.52 | 14.47 |
| 115 | LEU | N | 22.39 | 58.70 | −10.31 | 14.06 |
| 115 | LEU | CA | 21.03 | 58.17 | −10.28 | 13.80 |
| 115 | LEU | CB | 20.76 | 57.41 | −8.98 | 13.93 |
| 115 | LEU | CG | 21.61 | 56.17 | −8.70 | 14.04 |
| 115 | LEU | CD1 | 21.13 | 55.52 | −7.41 | 14.10 |
| 115 | LEU | CD2 | 21.51 | 55.20 | −9.86 | 14.12 |
| 115 | LEU | C | 20.10 | 59.37 | −10.38 | 13.57 |
| 115 | LEU | O | 20.43 | 60.46 | −9.93 | 13.48 |
| 116 | ASN | N | 18.93 | 59.16 | −10.98 | 13.35 |
| 116 | ASN | CA | 17.97 | 60.24 | −11.12 | 13.20 |
| 116 | ASN | CB | 17.26 | 60.13 | −12.47 | 13.27 |
| 116 | ASN | CG | 16.22 | 61.21 | −12.66 | 13.33 |
| 116 | ASN | OD1 | 16.14 | 62.15 | −11.88 | 13.34 |
| 116 | ASN | ND2 | 15.42 | 61.08 | −13.72 | 13.44 |
| 116 | ASN | C | 16.95 | 60.21 | −9.99 | 13.07 |
| 116 | ASN | O | 15.97 | 59.47 | −10.04 | 12.97 |
| 117 | PHE | N | 17.19 | 61.02 | −8.96 | 12.93 |
| 117 | PHE | CA | 16.31 | 61.08 | −7.80 | 12.85 |
| 117 | PHE | CB | 16.88 | 62.04 | −6.75 | 12.73 |
| 117 | PHE | CG | 18.07 | 61.50 | −5.98 | 12.57 |
| 117 | PHE | CD1 | 18.70 | 60.31 | −6.34 | 12.61 |
| 117 | PHE | CD2 | 18.55 | 62.20 | −4.88 | 12.63 |
| 117 | PHE | CE1 | 19.80 | 59.84 | −5.61 | 12.60 |
| 117 | PHE | CE2 | 19.64 | 61.74 | −4.15 | 12.63 |
| 117 | PHE | CZ | 20.27 | 60.56 | −4.51 | 12.51 |
| 117 | PHE | C | 14.89 | 61.53 | −8.15 | 12.94 |
| 117 | PHE | O | 13.96 | 61.27 | −7.39 | 12.85 |
| 118 | ASN | N | 14.74 | 62.20 | −9.29 | 13.08 |
| 118 | ASN | CA | 13.42 | 62.69 | −9.70 | 13.25 |
| 118 | ASN | CB | 13.56 | 63.90 | −10.63 | 13.52 |
| 118 | ASN | CG | 14.01 | 65.14 | −9.89 | 13.76 |
| 118 | ASN | OD1 | 13.76 | 65.30 | −8.70 | 13.95 |
| 118 | ASN | ND2 | 14.67 | 66.05 | −10.61 | 14.10 |
| 118 | ASN | C | 12.55 | 61.63 | −10.37 | 13.26 |
| 118 | ASN | O | 11.37 | 61.87 | −10.60 | 13.29 |
| 119 | ASN | N | 13.14 | 60.49 | −10.70 | 13.28 |
| 119 | ASN | CA | 12.38 | 59.41 | −11.32 | 13.31 |
| 119 | ASN | CB | 13.23 | 58.65 | −12.33 | 13.60 |
| 119 | ASN | CG | 12.45 | 57.59 | −13.09 | 13.77 |
| 119 | ASN | OD1 | 11.46 | 57.05 | −12.58 | 13.93 |
| 119 | ASN | ND2 | 12.89 | 57.27 | −14.30 | 13.98 |
| 119 | ASN | C | 11.99 | 58.48 | −10.18 | 13.19 |
| 119 | ASN | O | 12.73 | 57.56 | −9.83 | 13.04 |
| 120 | VAL | N | 10.83 | 58.73 | −9.58 | 13.16 |
| 120 | VAL | CA | 10.37 | 57.93 | −8.45 | 13.16 |
| 120 | VAL | CB | 9.05 | 58.49 | −7.88 | 13.13 |
| 120 | VAL | CG1 | 8.61 | 57.68 | −6.68 | 13.13 |
| 120 | VAL | CG2 | 9.25 | 59.95 | −7.49 | 13.18 |
| 120 | VAL | C | 10.21 | 56.45 | −8.80 | 13.24 |
| 120 | VAL | O | 10.56 | 55.58 | −8.01 | 13.11 |
| 121 | GLU | N | 9.70 | 56.17 | −10.00 | 13.43 |
| 121 | GLU | CA | 9.50 | 54.79 | −10.45 | 13.74 |
| 121 | GLU | CB | 8.87 | 54.80 | −11.84 | 14.20 |
| 121 | GLU | CG | 8.75 | 53.44 | −12.50 | 14.95 |
| 121 | GLU | CD | 8.17 | 53.53 | −13.90 | 15.35 |
| 121 | GLU | OE1 | 6.98 | 53.89 | −14.02 | 15.68 |
| 121 | GLU | OE2 | 8.90 | 53.25 | −14.87 | 15.83 |
| 121 | GLU | C | 10.81 | 54.01 | −10.47 | 13.63 |
| 121 | GLU | O | 10.87 | 52.86 | −10.03 | 13.65 |
| 122 | GLN | N | 11.87 | 54.63 | −10.98 | 13.58 |
| 122 | GLN | CA | 13.17 | 53.96 | −11.03 | 13.52 |
| 122 | GLN | CB | 14.10 | 54.68 | −12.01 | 13.95 |
| 122 | GLN | CG | 15.49 | 54.07 | −12.12 | 14.62 |
| 122 | GLN | CD | 15.46 | 52.60 | −12.51 | 14.97 |
| 122 | GLN | OE1 | 14.90 | 52.23 | −13.53 | 15.38 |
| 122 | GLN | NE2 | 16.08 | 51.76 | −11.68 | 15.26 |
| 122 | GLN | C | 13.81 | 53.91 | −9.65 | 13.21 |
| 122 | GLN | O | 14.38 | 52.89 | −9.26 | 13.21 |
| 123 | MET | N | 13.72 | 55.01 | −8.90 | 12.90 |
| 123 | MET | CA | 14.29 | 55.03 | −7.56 | 12.67 |
| 123 | MET | CB | 14.09 | 56.40 | −6.90 | 12.51 |
| 123 | MET | CG | 14.97 | 57.51 | −7.47 | 12.35 |
| 123 | MET | SD | 16.75 | 57.16 | −7.33 | 12.05 |
| 123 | MET | CE | 16.98 | 57.34 | −5.54 | 12.42 |
| 123 | MET | C | 13.68 | 53.94 | −6.69 | 12.60 |
| 123 | MET | O | 14.37 | 53.33 | −5.87 | 12.50 |
| 124 | LYS | N | 12.39 | 53.68 | −6.87 | 12.60 |
| 124 | LYS | CA | 11.68 | 52.65 | −6.11 | 12.69 |
| 124 | LYS | CB | 10.24 | 52.54 | −6.59 | 12.52 |
| 124 | LYS | CG | 9.36 | 51.61 | −5.76 | 12.32 |
| 124 | LYS | CD | 8.01 | 51.42 | −6.43 | 12.17 |
| 124 | LYS | CE | 6.98 | 50.79 | −5.49 | 11.98 |
| 124 | LYS | NZ | 6.48 | 51.77 | −4.49 | 11.91 |
| 124 | LYS | C | 12.38 | 51.30 | −6.27 | 12.87 |
| 124 | LYS | O | 12.47 | 50.53 | −5.32 | 12.79 |
| 125 | LYS | N | 12.87 | 51.03 | −7.47 | 13.18 |
| 125 | LYS | CA | 13.55 | 49.77 | −7.74 | 13.55 |
| 125 | LYS | CB | 13.91 | 49.66 | −9.23 | 13.97 |
| 125 | LYS | CG | 12.71 | 49.63 | −10.16 | 14.55 |
| 125 | LYS | CD | 13.15 | 49.48 | −11.61 | 15.02 |
| 125 | LYS | CE | 11.97 | 49.52 | −12.56 | 15.38 |
| 125 | LYS | NZ | 11.03 | 48.39 | −12.34 | 15.77 |
| 125 | LYS | C | 14.81 | 49.63 | −6.89 | 13.51 |
| 125 | LYS | O | 15.15 | 48.53 | −6.45 | 13.71 |
| 126 | TYR | N | 15.50 | 50.74 | −6.66 | 13.39 |
| 126 | TYR | CA | 16.71 | 50.74 | −5.86 | 13.37 |
| 126 | TYR | CB | 17.57 | 51.97 | −6.16 | 13.49 |
| 126 | TYR | CG | 18.07 | 52.10 | −7.58 | 13.69 |
| 126 | TYR | CD1 | 18.82 | 51.08 | −8.17 | 13.83 |
| 126 | TYR | CE1 | 19.33 | 51.22 | −9.45 | 14.02 |
| 126 | TYR | CD2 | 17.84 | 53.26 | −8.31 | 13.79 |
| 126 | TYR | CE2 | 18.35 | 53.41 | −9.59 | 13.97 |
| 126 | TYR | CZ | 19.10 | 52.39 | −10.16 | 14.00 |
| 126 | TYR | OH | 19.62 | 52.54 | −11.42 | 14.25 |
| 126 | TYR | C | 16.44 | 50.75 | −4.35 | 13.31 |
| 126 | TYR | O | 17.02 | 49.96 | −3.60 | 13.26 |
| 127 | THR | N | 15.57 | 51.66 | −3.92 | 13.31 |
| 127 | THR | CA | 15.28 | 51.82 | −2.51 | 13.40 |
| 127 | THR | CB | 14.58 | 53.17 | −2.25 | 13.17 |
| 127 | THR | OG1 | 13.30 | 53.17 | −2.89 | 12.85 |
| 127 | THR | CG2 | 15.42 | 54.31 | −2.80 | 13.12 |
| 127 | THR | C | 14.45 | 50.73 | −1.84 | 13.70 |
| 127 | THR | O | 14.74 | 50.33 | −0.71 | 13.71 |
| 128 | GLN | N | 13.43 | 50.24 | −2.53 | 14.09 |
| 128 | GLN | CA | 12.57 | 49.23 | −1.95 | 14.59 |
| 128 | GLN | CB | 11.14 | 49.41 | −2.48 | 14.66 |
| 128 | GLN | CG | 10.66 | 50.88 | −2.43 | 14.80 |
| 128 | GLN | CD | 10.73 | 51.49 | −1.04 | 14.81 |
| 128 | GLN | OE1 | 10.02 | 51.07 | −0.12 | 14.89 |
| 128 | GLN | NE2 | 11.59 | 52.50 | −0.88 | 14.86 |
| 128 | GLN | C | 13.08 | 47.80 | −2.18 | 14.81 |
| 128 | GLN | O | 12.32 | 46.84 | −2.06 | 15.04 |
| 129 | SER | N | 14.36 | 47.69 | −2.50 | 14.94 |
| 129 | SER | CA | 15.02 | 46.40 | −2.73 | 14.97 |
| 129 | SER | CB | 15.19 | 46.12 | −4.23 | 15.26 |
| 129 | SER | OG | 16.18 | 46.96 | −4.80 | 15.90 |
| 129 | SER | C | 16.41 | 46.46 | −2.07 | 14.69 |
| 129 | SER | O | 17.19 | 45.52 | −2.16 | 14.85 |
| 130 | TYR | N | 16.68 | 47.59 | −1.41 | 14.25 |
| 130 | TYR | CA | 17.95 | 47.81 | −0.74 | 13.73 |
| 130 | TYR | CB | 18.00 | 49.25 | −0.19 | 13.33 |
| 130 | TYR | CG | 19.21 | 49.53 | 0.66 | 12.81 |
| 130 | TYR | CD1 | 20.46 | 49.72 | 0.07 | 12.65 |
| 130 | TYR | CE1 | 21.59 | 49.95 | 0.85 | 12.38 |
| 130 | TYR | CD2 | 19.12 | 49.58 | 2.05 | 12.58 |
| 130 | TYR | CE2 | 20.24 | 49.80 | 2.84 | 12.30 |
| 130 | TYR | CZ | 21.48 | 49.98 | 2.23 | 12.22 |
| 130 | TYR | OH | 22.60 | 50.19 | 3.00 | 11.91 |
| 130 | TYR | C | 18.24 | 46.84 | 0.40 | 13.56 |
| 130 | TYR | O | 17.35 | 46.50 | 1.18 | 13.58 |
| 131 | SER | N | 19.49 | 46.40 | 0.50 | 13.36 |
| 131 | SER | CA | 19.91 | 45.49 | 1.55 | 13.18 |
| 131 | SER | CB | 20.16 | 44.09 | 1.00 | 13.41 |
| 131 | SER | OG | 20.52 | 43.19 | 2.04 | 13.96 |
| 131 | SER | C | 21.20 | 46.05 | 2.15 | 12.83 |
| 131 | SER | O | 22.20 | 46.22 | 1.45 | 12.97 |
| 132 | GLY | N | 21.18 | 46.33 | 3.45 | 12.41 |
| 132 | GLY | CA | 22.36 | 46.86 | 4.10 | 11.86 |
| 132 | GLY | C | 21.99 | 47.77 | 5.25 | 11.44 |
| 132 | GLY | O | 20.83 | 47.85 | 5.65 | 11.38 |
| 133 | GLY | N | 23.00 | 48.46 | 5.78 | 11.05 |
| 133 | GLY | CA | 22.75 | 49.35 | 6.89 | 10.65 |
| 133 | GLY | C | 21.84 | 50.51 | 6.53 | 10.35 |
| 133 | GLY | O | 21.95 | 51.09 | 5.45 | 10.18 |
| 134 | LEU | N | 20.92 | 50.84 | 7.43 | 10.20 |
| 134 | LEU | CA | 20.02 | 51.95 | 7.22 | 10.11 |
| 134 | LEU | CB | 18.74 | 51.52 | 6.49 | 10.59 |
| 134 | LEU | CG | 17.67 | 50.71 | 7.23 | 11.09 |
| 134 | LEU | CD1 | 16.41 | 50.65 | 6.39 | 11.43 |
| 134 | LEU | CD2 | 18.18 | 49.31 | 7.52 | 11.43 |
| 134 | LEU | C | 19.68 | 52.58 | 8.56 | 9.79 |
| 134 | LEU | O | 19.74 | 51.92 | 9.61 | 9.82 |
| 135 | ALA | N | 19.33 | 53.86 | 8.52 | 9.46 |
| 135 | ALA | CA | 18.98 | 54.61 | 9.72 | 9.24 |
| 135 | ALA | CB | 20.18 | 55.40 | 10.20 | 9.59 |
| 135 | ALA | C | 17.80 | 55.53 | 9.45 | 8.99 |
| 135 | ALA | O | 17.60 | 56.01 | 8.33 | 8.78 |
| 136 | ASP | N | 17.05 | 55.79 | 10.52 | 8.80 |
| 136 | ASP | CA | 15.84 | 56.59 | 10.49 | 8.77 |
| 136 | ASP | CB | 14.66 | 55.61 | 10.59 | 9.03 |
| 136 | ASP | CG | 13.31 | 56.29 | 10.75 | 9.23 |
| 136 | ASP | OD1 | 12.31 | 55.55 | 10.93 | 9.96 |
| 136 | ASP | OD2 | 13.24 | 57.53 | 10.69 | 9.15 |
| 136 | ASP | C | 15.85 | 57.54 | 11.70 | 8.70 |
| 136 | ASP | O | 15.96 | 57.09 | 12.83 | 8.63 |
| 137 | SER | N | 15.77 | 58.85 | 11.45 | 8.61 |
| 137 | SER | CA | 15.73 | 59.81 | 12.56 | 8.68 |
| 137 | SER | CB | 17.12 | 59.99 | 13.19 | 8.66 |
| 137 | SER | OG | 17.10 | 61.03 | 14.17 | 8.77 |
| 137 | SER | C | 15.17 | 61.18 | 12.23 | 8.73 |
| 137 | SER | O | 15.69 | 61.90 | 11.37 | 8.86 |
| 138 | CYS | N | 14.10 | 61.53 | 12.94 | 8.95 |
| 138 | CYS | CA | 13.46 | 62.83 | 12.80 | 8.88 |
| 138 | CYS | CB | 11.96 | 62.71 | 13.04 | 9.06 |
| 138 | CYS | SG | 11.06 | 64.28 | 12.96 | 8.93 |
| 138 | CYS | C | 14.04 | 63.74 | 13.89 | 8.88 |
| 138 | CYS | O | 14.20 | 63.31 | 15.03 | 8.82 |
| 139 | TRP | N | 14.40 | 64.97 | 13.53 | 8.87 |
| 139 | TRP | CA | 14.88 | 65.92 | 14.53 | 8.96 |
| 139 | TRP | CB | 16.40 | 66.13 | 14.46 | 9.16 |
| 139 | TRP | CG | 16.97 | 66.42 | 15.84 | 9.27 |
| 139 | TRP | CD2 | 17.03 | 65.50 | 16.94 | 9.35 |
| 139 | TRP | CE2 | 17.51 | 66.22 | 18.06 | 9.39 |
| 139 | TRP | CE3 | 16.72 | 64.14 | 17.09 | 9.43 |
| 139 | TRP | CD1 | 17.41 | 67.62 | 16.32 | 9.45 |
| 139 | TRP | NE1 | 17.74 | 67.51 | 17.65 | 9.45 |
| 139 | TRP | CZ2 | 17.68 | 65.62 | 19.31 | 9.52 |
| 139 | TRP | CZ3 | 16.89 | 63.55 | 18.33 | 9.48 |
| 139 | TRP | CH2 | 17.37 | 64.29 | 19.42 | 9.52 |
| 139 | TRP | C | 14.13 | 67.23 | 14.28 | 8.94 |
| 139 | TRP | O | 13.82 | 67.57 | 13.13 | 8.90 |
| 140 | ALA | N | 13.82 | 67.95 | 15.35 | 9.04 |
| 140 | ALA | CA | 13.07 | 69.20 | 15.26 | 9.25 |
| 140 | ALA | CB | 12.02 | 69.26 | 16.36 | 9.27 |
| 140 | ALA | C | 13.97 | 70.42 | 15.33 | 9.44 |
| 140 | ALA | O | 14.96 | 70.43 | 16.07 | 9.50 |
| 141 | TYR | N | 13.59 | 71.45 | 14.58 | 9.66 |
| 141 | TYR | CA | 14.34 | 72.70 | 14.51 | 10.07 |
| 141 | TYR | CB | 15.02 | 72.83 | 13.14 | 10.02 |
| 141 | TYR | CG | 16.00 | 71.73 | 12.85 | 9.93 |
| 141 | TYR | CD1 | 15.57 | 70.49 | 12.37 | 9.82 |
| 141 | TYR | CE1 | 16.47 | 69.45 | 12.17 | 9.75 |
| 141 | TYR | CD2 | 17.35 | 71.91 | 13.12 | 9.96 |
| 141 | TYR | CE2 | 18.26 | 70.89 | 12.93 | 9.90 |
| 141 | TYR | CZ | 17.82 | 69.66 | 12.46 | 9.81 |
| 141 | TYR | OH | 18.72 | 68.64 | 12.31 | 9.93 |
| 141 | TYR | C | 13.49 | 73.93 | 14.72 | 10.38 |
| 141 | TYR | O | 12.40 | 74.05 | 14.15 | 10.33 |
| 142 | ALA | N | 13.99 | 74.85 | 15.53 | 10.80 |
| 142 | ALA | CA | 13.31 | 76.11 | 15.76 | 11.26 |
| 142 | ALA | CB | 13.54 | 76.59 | 17.19 | 11.40 |
| 142 | ALA | C | 13.95 | 77.08 | 14.77 | 11.58 |
| 142 | ALA | O | 15.17 | 77.25 | 14.77 | 11.70 |
| 143 | VAL | N | 13.14 | 77.68 | 13.90 | 11.93 |
| 143 | VAL | CA | 13.66 | 78.63 | 12.93 | 12.34 |
| 143 | VAL | CB | 12.73 | 78.75 | 11.71 | 12.36 |
| 143 | VAL | CG1 | 13.32 | 79.72 | 10.69 | 12.51 |
| 143 | VAL | CG2 | 12.54 | 77.37 | 11.08 | 12.44 |
| 143 | VAL | C | 13.77 | 79.99 | 13.61 | 12.59 |
| 143 | VAL | O | 12.77 | 80.52 | 14.10 | 12.55 |
| 144 | GLY | N | 14.97 | 80.54 | 13.65 | 12.98 |
| 144 | GLY | CA | 15.16 | 81.82 | 14.30 | 13.52 |
| 144 | GLY | C | 14.93 | 81.69 | 15.80 | 13.88 |
| 144 | GLY | O | 15.43 | 80.76 | 16.43 | 14.06 |
| 145 | THR | N | 14.15 | 82.61 | 16.36 | 14.21 |
| 145 | THR | CA | 13.85 | 82.58 | 17.78 | 14.53 |
| 145 | THR | CB | 13.96 | 83.99 | 18.39 | 14.71 |
| 145 | THR | OG1 | 15.27 | 84.52 | 18.15 | 15.01 |
| 145 | THR | CG2 | 13.70 | 83.95 | 19.89 | 14.89 |
| 145 | THR | C | 12.44 | 82.04 | 18.01 | 14.55 |
| 145 | THR | O | 11.46 | 82.67 | 17.63 | 14.71 |
| 146 | PRO | N | 12.33 | 80.86 | 18.63 | 14.52 |
| 146 | PRO | CD | 13.39 | 79.95 | 19.09 | 14.57 |
| 146 | PRO | CA | 11.01 | 80.28 | 18.88 | 14.42 |
| 146 | PRO | CB | 11.33 | 78.82 | 19.16 | 14.52 |
| 146 | PRO | CG | 12.62 | 78.92 | 19.91 | 14.55 |
| 146 | PRO | C | 10.33 | 80.95 | 20.07 | 14.25 |
| 146 | PRO | O | 10.99 | 81.51 | 20.93 | 14.25 |
| 147 | SER | N | 9.00 | 80.90 | 20.11 | 14.07 |
| 147 | SER | CA | 8.28 | 81.46 | 21.24 | 13.80 |
| 147 | SER | CB | 6.78 | 81.49 | 20.97 | 14.00 |
| 147 | SER | OG | 6.26 | 80.17 | 20.91 | 14.19 |
| 147 | SER | C | 8.59 | 80.48 | 22.37 | 13.53 |
| 147 | SER | O | 9.00 | 79.35 | 22.11 | 13.29 |
| 148 | GLU | N | 8.41 | 80.90 | 23.62 | 13.32 |
| 148 | GLU | CA | 8.70 | 80.00 | 24.73 | 13.24 |
| 148 | GLU | CB | 8.60 | 80.75 | 26.05 | 13.36 |
| 148 | GLU | CG | 9.68 | 81.81 | 26.20 | 13.61 |
| 148 | GLU | CD | 9.57 | 82.59 | 27.49 | 13.71 |
| 148 | GLU | OE1 | 9.71 | 81.98 | 28.57 | 13.78 |
| 148 | GLU | OE2 | 9.33 | 83.81 | 27.42 | 13.92 |
| 148 | GLU | C | 7.76 | 78.80 | 24.70 | 13.12 |
| 148 | GLU | O | 8.15 | 77.70 | 25.10 | 12.97 |
| 149 | GLU | N | 6.54 | 79.00 | 24.21 | 13.11 |
| 149 | GLU | CA | 5.58 | 77.90 | 24.14 | 13.19 |
| 149 | GLU | CB | 4.21 | 78.42 | 23.72 | 13.76 |
| 149 | GLU | CG | 3.07 | 77.48 | 24.09 | 14.60 |
| 149 | GLU | CD | 1.71 | 77.99 | 23.65 | 15.03 |
| 149 | GLU | OE1 | 1.52 | 79.22 | 23.57 | 15.47 |
| 149 | GLU | OE2 | 0.82 | 77.14 | 23.39 | 15.52 |
| 149 | GLU | C | 6.08 | 76.87 | 23.12 | 12.86 |
| 149 | GLU | O | 5.97 | 75.67 | 23.34 | 12.69 |
| 150 | VAL | N | 6.63 | 77.35 | 22.01 | 12.58 |
| 150 | VAL | CA | 7.16 | 76.45 | 21.00 | 12.30 |
| 150 | VAL | CB | 7.58 | 77.22 | 19.72 | 12.37 |
| 150 | VAL | CG1 | 8.42 | 76.32 | 18.82 | 12.52 |
| 150 | VAL | CG2 | 6.34 | 77.69 | 18.97 | 12.53 |
| 150 | VAL | C | 8.36 | 75.68 | 21.54 | 12.02 |
| 150 | VAL | O | 8.47 | 74.47 | 21.34 | 11.87 |
| 151 | LYS | N | 9.26 | 76.37 | 22.24 | 11.83 |
| 151 | LYS | CA | 10.43 | 75.69 | 22.78 | 11.72 |
| 151 | LYS | CB | 11.46 | 76.69 | 23.32 | 12.26 |
| 151 | LYS | CG | 12.82 | 76.06 | 23.63 | 12.99 |
| 151 | LYS | CD | 13.45 | 75.41 | 22.40 | 13.68 |
| 151 | LYS | CE | 14.77 | 74.73 | 22.75 | 14.14 |
| 151 | LYS | NZ | 15.36 | 74.02 | 21.59 | 14.71 |
| 151 | LYS | C | 10.03 | 74.69 | 23.86 | 11.35 |
| 151 | LYS | O | 10.66 | 73.65 | 24.00 | 11.14 |
| 152 | ASN | N | 8.99 | 75.00 | 24.63 | 11.06 |
| 152 | ASN | CA | 8.52 | 74.08 | 25.66 | 10.82 |
| 152 | ASN | CB | 7.36 | 74.67 | 26.47 | 11.13 |
| 152 | ASN | CG | 7.82 | 75.47 | 27.67 | 11.31 |
| 152 | ASN | OD1 | 8.96 | 75.34 | 28.12 | 11.60 |
| 152 | ASN | ND2 | 6.92 | 76.28 | 28.21 | 11.53 |
| 152 | ASN | C | 8.05 | 72.79 | 24.99 | 10.55 |
| 152 | ASN | O | 8.35 | 71.69 | 25.46 | 10.40 |
| 153 | LEU | N | 7.31 | 72.93 | 23.90 | 10.32 |
| 153 | LEU | CA | 6.81 | 71.77 | 23.16 | 10.12 |
| 153 | LEU | CB | 5.97 | 72.22 | 21.97 | 10.23 |
| 153 | LEU | CG | 5.61 | 71.14 | 20.95 | 10.34 |
| 153 | LEU | CD1 | 4.71 | 70.09 | 21.57 | 10.49 |
| 153 | LEU | CD2 | 4.93 | 71.78 | 19.75 | 10.48 |
| 153 | LEU | C | 7.99 | 70.93 | 22.68 | 9.96 |
| 153 | LEU | O | 7.97 | 69.70 | 22.80 | 9.73 |
| 154 | MET | N | 9.01 | 71.59 | 22.13 | 9.91 |
| 154 | MET | CA | 10.18 | 70.88 | 21.64 | 9.97 |
| 154 | MET | CB | 11.18 | 71.86 | 21.02 | 10.08 |
| 154 | MET | CG | 10.70 | 72.52 | 19.73 | 10.30 |
| 154 | MET | SD | 11.82 | 73.80 | 19.12 | 10.24 |
| 154 | MET | CE | 13.20 | 72.80 | 18.54 | 10.71 |
| 154 | MET | C | 10.87 | 70.11 | 22.76 | 9.93 |
| 154 | MET | O | 11.15 | 68.92 | 22.63 | 9.72 |
| 155 | ASP | N | 11.12 | 70.79 | 23.88 | 10.03 |
| 155 | ASP | CA | 11.79 | 70.14 | 25.00 | 10.21 |
| 155 | ASP | CB | 12.08 | 71.16 | 26.11 | 10.64 |
| 155 | ASP | CG | 13.20 | 72.12 | 25.74 | 10.98 |
| 155 | ASP | OD1 | 13.82 | 71.95 | 24.67 | 11.30 |
| 155 | ASP | OD2 | 13.46 | 73.05 | 26.54 | 11.42 |
| 155 | ASP | C | 10.99 | 68.98 | 25.57 | 10.13 |
| 155 | ASP | O | 11.56 | 67.94 | 25.90 | 10.06 |
| 156 | VAL | N | 9.68 | 69.15 | 25.70 | 10.11 |
| 156 | VAL | CA | 8.84 | 68.08 | 26.23 | 10.17 |
| 156 | VAL | CB | 7.38 | 68.56 | 26.44 | 10.38 |
| 156 | VAL | CG1 | 6.47 | 67.37 | 26.73 | 10.79 |
| 156 | VAL | CG2 | 7.32 | 69.55 | 27.59 | 10.71 |
| 156 | VAL | C | 8.86 | 66.87 | 25.31 | 9.96 |
| 156 | VAL | O | 8.95 | 65.73 | 25.77 | 9.93 |
| 157 | THR | N | 8.79 | 67.11 | 24.00 | 9.79 |
| 157 | THR | CA | 8.81 | 66.02 | 23.04 | 9.63 |
| 157 | THR | CB | 8.59 | 66.55 | 21.61 | 9.54 |
| 157 | THR | OG1 | 7.35 | 67.27 | 21.56 | 9.58 |
| 157 | THR | CG2 | 8.55 | 65.41 | 20.61 | 9.60 |
| 157 | THR | C | 10.14 | 65.27 | 23.09 | 9.60 |
| 157 | THR | O | 10.17 | 64.03 | 23.11 | 9.51 |
| 158 | LYS | N | 11.23 | 66.01 | 23.13 | 9.74 |
| 158 | LYS | CA | 12.55 | 65.40 | 23.17 | 9.93 |
| 158 | LYS | CB | 13.63 | 66.48 | 23.03 | 10.23 |
| 158 | LYS | CG | 15.04 | 65.90 | 22.88 | 10.73 |
| 158 | LYS | CD | 16.06 | 66.98 | 22.58 | 11.25 |
| 158 | LYS | CE | 16.24 | 67.93 | 23.75 | 11.60 |
| 158 | LYS | NZ | 17.11 | 69.09 | 23.37 | 11.97 |
| 158 | LYS | C | 12.77 | 64.60 | 24.46 | 9.94 |
| 158 | LYS | O | 13.29 | 63.49 | 24.42 | 9.78 |
| 159 | GLU | N | 12.38 | 65.17 | 25.59 | 10.09 |
| 159 | GLU | CA | 12.56 | 64.46 | 26.86 | 10.37 |
| 159 | GLU | CB | 12.29 | 65.39 | 28.04 | 10.95 |
| 159 | GLU | CG | 12.54 | 64.76 | 29.41 | 12.07 |
| 159 | GLU | CD | 13.92 | 64.13 | 29.56 | 12.62 |
| 159 | GLU | OE1 | 14.87 | 64.60 | 28.91 | 13.15 |
| 159 | GLU | OE2 | 14.04 | 63.16 | 30.34 | 13.36 |
| 159 | GLU | C | 11.65 | 63.24 | 26.91 | 10.14 |
| 159 | GLU | O | 12.04 | 62.20 | 27.46 | 10.08 |
| 160 | ALA | N | 10.45 | 63.34 | 26.35 | 10.00 |
| 160 | ALA | CA | 9.54 | 62.20 | 26.33 | 9.82 |
| 160 | ALA | CB | 8.22 | 62.59 | 25.68 | 9.95 |
| 160 | ALA | C | 10.21 | 61.07 | 25.56 | 9.68 |
| 160 | ALA | O | 10.13 | 59.90 | 25.95 | 9.59 |
| 161 | MET | N | 10.87 | 61.41 | 24.45 | 9.62 |
| 161 | MET | CA | 11.56 | 60.41 | 23.65 | 9.58 |
| 161 | MET | CB | 12.22 | 61.06 | 22.43 | 9.65 |
| 161 | MET | CG | 13.08 | 60.09 | 21.62 | 9.78 |
| 161 | MET | SD | 14.20 | 60.91 | 20.46 | 9.87 |
| 161 | MET | CE | 15.26 | 61.81 | 21.61 | 10.09 |
| 161 | MET | C | 12.63 | 59.72 | 24.49 | 9.52 |
| 161 | MET | O | 12.71 | 58.49 | 24.52 | 9.37 |
| 162 | TYR | N | 13.46 | 60.51 | 25.18 | 9.65 |
| 162 | TYR | CA | 14.52 | 59.92 | 26.00 | 9.90 |
| 162 | TYR | CB | 15.47 | 61.01 | 26.50 | 10.02 |
| 162 | TYR | CG | 16.46 | 61.47 | 25.44 | 10.22 |
| 162 | TYR | CD1 | 17.38 | 60.58 | 24.88 | 10.36 |
| 162 | TYR | CE1 | 18.29 | 61.00 | 23.91 | 10.49 |
| 162 | TYR | CD2 | 16.48 | 62.79 | 25.00 | 10.35 |
| 162 | TYR | CE2 | 17.39 | 63.22 | 24.03 | 10.60 |
| 162 | TYR | CZ | 18.29 | 62.32 | 23.49 | 10.53 |
| 162 | TYR | OH | 19.19 | 62.74 | 22.54 | 10.72 |
| 162 | TYR | C | 13.96 | 59.10 | 27.16 | 9.97 |
| 162 | TYR | O | 14.57 | 58.11 | 27.56 | 9.95 |
| 163 | LYS | N | 12.80 | 59.50 | 27.69 | 10.15 |
| 163 | LYS | CA | 12.17 | 58.74 | 28.77 | 10.33 |
| 163 | LYS | CB | 10.93 | 59.47 | 29.29 | 10.83 |
| 163 | LYS | CG | 11.22 | 60.67 | 30.17 | 11.56 |
| 163 | LYS | CD | 11.79 | 60.26 | 31.52 | 12.21 |
| 163 | LYS | CE | 11.99 | 61.46 | 32.42 | 12.68 |
| 163 | LYS | NZ | 12.50 | 61.05 | 33.76 | 13.19 |
| 163 | LYS | C | 11.77 | 57.37 | 28.22 | 10.16 |
| 163 | LYS | O | 11.86 | 56.35 | 28.91 | 10.24 |
| 164 | GLY | N | 11.31 | 57.36 | 26.97 | 10.06 |
| 164 | GLY | CA | 10.92 | 56.11 | 26.34 | 10.01 |
| 164 | GLY | C | 12.13 | 55.22 | 26.11 | 9.95 |
| 164 | GLY | O | 12.10 | 54.02 | 26.40 | 9.88 |
| 165 | ILE | N | 13.20 | 55.80 | 25.58 | 10.06 |
| 165 | ILE | CA | 14.41 | 55.04 | 25.30 | 10.27 |
| 165 | ILE | CB | 15.48 | 55.94 | 24.62 | 10.27 |
| 165 | ILE | CG2 | 16.79 | 55.18 | 24.47 | 10.42 |
| 165 | ILE | CG1 | 14.96 | 56.41 | 23.26 | 10.26 |
| 165 | ILE | CD1 | 15.86 | 57.41 | 22.55 | 10.34 |
| 165 | ILE | C | 14.98 | 54.41 | 26.57 | 10.46 |
| 165 | ILE | O | 15.46 | 53.28 | 26.55 | 10.39 |
| 166 | GLU | N | 14.89 | 55.14 | 27.68 | 10.85 |
| 166 | GLU | CA | 15.39 | 54.65 | 28.96 | 11.33 |
| 166 | GLU | CB | 15.18 | 55.72 | 30.03 | 12.06 |
| 166 | GLU | CG | 15.56 | 55.31 | 31.44 | 13.23 |
| 166 | GLU | CD | 15.44 | 56.47 | 32.41 | 13.85 |
| 166 | GLU | OE1 | 14.34 | 57.06 | 32.49 | 14.29 |
| 166 | GLU | OE2 | 16.44 | 56.78 | 33.08 | 14.48 |
| 166 | GLU | C | 14.71 | 53.34 | 29.39 | 11.21 |
| 166 | GLU | O | 15.30 | 52.52 | 30.09 | 11.16 |
| 167 | GLN | N | 13.47 | 53.16 | 28.95 | 11.18 |
| 167 | GLN | CA | 12.70 | 51.96 | 29.28 | 11.30 |
| 167 | GLN | CB | 11.20 | 52.26 | 29.22 | 11.48 |
| 167 | GLN | CG | 10.70 | 53.24 | 30.25 | 11.91 |
| 167 | GLN | CD | 10.87 | 52.72 | 31.66 | 12.19 |
| 167 | GLN | OE1 | 10.61 | 51.54 | 31.93 | 12.44 |
| 167 | GLN | NE2 | 11.30 | 53.58 | 32.57 | 12.53 |
| 167 | GLN | C | 12.99 | 50.78 | 28.36 | 11.29 |
| 167 | GLN | O | 12.55 | 49.66 | 28.63 | 11.23 |
| 168 | ALA | N | 13.72 | 51.03 | 27.28 | 11.32 |
| 168 | ALA | CA | 14.03 | 49.98 | 26.31 | 11.53 |
| 168 | ALA | CB | 14.30 | 50.61 | 24.94 | 11.49 |
| 168 | ALA | C | 15.21 | 49.11 | 26.73 | 11.71 |
| 168 | ALA | O | 16.27 | 49.13 | 26.10 | 11.72 |
| 169 | VAL | N | 15.00 | 48.34 | 27.79 | 11.97 |
| 169 | VAL | CA | 16.03 | 47.45 | 28.31 | 12.38 |
| 169 | VAL | CB | 16.37 | 47.78 | 29.78 | 12.47 |
| 169 | VAL | CG1 | 16.94 | 49.19 | 29.88 | 12.59 |
| 169 | VAL | CG2 | 15.13 | 47.64 | 30.66 | 12.56 |
| 169 | VAL | C | 15.56 | 46.00 | 28.23 | 12.64 |
| 169 | VAL | O | 14.36 | 45.72 | 28.23 | 12.60 |
| 170 | VAL | N | 16.51 | 45.07 | 28.15 | 12.98 |
| 170 | VAL | CA | 16.19 | 43.65 | 28.07 | 13.37 |
| 170 | VAL | CB | 17.47 | 42.78 | 28.14 | 13.53 |
| 170 | VAL | CG1 | 17.09 | 41.30 | 28.18 | 13.74 |
| 170 | VAL | CG2 | 18.36 | 43.07 | 26.95 | 13.67 |
| 170 | VAL | C | 15.28 | 43.28 | 29.22 | 13.50 |
| 170 | VAL | O | 15.53 | 43.65 | 30.37 | 13.53 |
| 171 | GLY | N | 14.22 | 42.55 | 28.93 | 13.57 |
| 171 | GLY | CA | 13.31 | 42.15 | 29.98 | 13.52 |
| 171 | GLY | C | 12.11 | 43.06 | 30.14 | 13.33 |
| 171 | GLY | O | 11.06 | 42.61 | 30.61 | 13.59 |
| 172 | ASN | N | 12.25 | 44.33 | 29.79 | 12.99 |
| 172 | ASN | CA | 11.12 | 45.25 | 29.87 | 12.51 |
| 172 | ASN | CB | 11.57 | 46.71 | 29.79 | 12.65 |
| 172 | ASN | CG | 11.60 | 47.40 | 31.14 | 12.73 |
| 172 | ASN | OD1 | 11.69 | 48.62 | 31.23 | 12.92 |
| 172 | ASN | ND2 | 11.54 | 46.61 | 32.21 | 12.87 |
| 172 | ASN | C | 10.21 | 44.91 | 28.71 | 12.15 |
| 172 | ASN | O | 10.48 | 43.98 | 27.95 | 12.08 |
| 173 | ARG | N | 9.12 | 45.66 | 28.56 | 11.79 |
| 173 | ARG | CA | 8.17 | 45.38 | 27.50 | 11.45 |
| 173 | ARG | CB | 6.94 | 44.72 | 28.13 | 11.89 |
| 173 | ARG | CG | 7.34 | 43.52 | 28.98 | 12.50 |
| 173 | ARG | CD | 6.19 | 42.91 | 29.74 | 12.99 |
| 173 | ARG | NE | 5.21 | 42.31 | 28.85 | 13.46 |
| 173 | ARG | CZ | 4.36 | 41.35 | 29.21 | 13.68 |
| 173 | ARG | NH1 | 4.37 | 40.88 | 30.45 | 13.99 |
| 173 | ARG | NH2 | 3.49 | 40.88 | 28.33 | 13.84 |
| 173 | ARG | C | 7.79 | 46.64 | 26.73 | 10.99 |
| 173 | ARG | O | 8.03 | 47.75 | 27.19 | 10.70 |
| 174 | ILE | N | 7.23 | 46.45 | 25.54 | 10.61 |
| 174 | ILE | CA | 6.84 | 47.58 | 24.69 | 10.33 |
| 174 | ILE | CB | 6.09 | 47.08 | 23.42 | 10.36 |
| 174 | ILE | CG2 | 5.40 | 48.23 | 22.72 | 10.42 |
| 174 | ILE | CG1 | 7.08 | 46.38 | 22.47 | 10.51 |
| 174 | ILE | CD1 | 8.04 | 47.31 | 21.74 | 10.67 |
| 174 | ILE | C | 5.97 | 48.59 | 25.45 | 10.11 |
| 174 | ILE | O | 6.12 | 49.80 | 25.27 | 9.86 |
| 175 | GLY | N | 5.08 | 48.09 | 26.31 | 10.06 |
| 175 | GLY | CA | 4.21 | 48.97 | 27.08 | 10.15 |
| 175 | GLY | C | 4.95 | 49.89 | 28.05 | 10.16 |
| 175 | GLY | O | 4.45 | 50.97 | 28.38 | 10.15 |
| 176 | ASP | N | 6.12 | 49.47 | 28.52 | 10.20 |
| 176 | ASP | CA | 6.88 | 50.30 | 29.44 | 10.27 |
| 176 | ASP | CB | 8.10 | 49.55 | 29.98 | 10.52 |
| 176 | ASP | CG | 7.71 | 48.37 | 30.84 | 10.72 |
| 176 | ASP | OD1 | 6.85 | 48.55 | 31.73 | 11.08 |
| 176 | ASP | OD2 | 8.25 | 47.26 | 30.63 | 10.93 |
| 176 | ASP | C | 7.33 | 51.57 | 28.73 | 10.15 |
| 176 | ASP | O | 7.37 | 52.65 | 29.31 | 10.05 |
| 177 | ILE | N | 7.68 | 51.42 | 27.45 | 10.09 |
| 177 | ILE | CA | 8.12 | 52.55 | 26.64 | 10.04 |
| 177 | ILE | CB | 8.61 | 52.08 | 25.26 | 10.09 |
| 177 | ILE | CG2 | 8.83 | 53.27 | 24.34 | 10.11 |
| 177 | ILE | CG1 | 9.90 | 51.26 | 25.43 | 10.15 |
| 177 | ILE | CD1 | 10.30 | 50.48 | 24.19 | 10.22 |
| 177 | ILE | C | 6.96 | 53.53 | 26.46 | 10.02 |
| 177 | ILE | O | 7.09 | 54.72 | 26.72 | 9.90 |
| 178 | GLY | N | 5.81 | 53.01 | 26.03 | 10.04 |
| 178 | GLY | CA | 4.65 | 53.85 | 25.81 | 10.16 |
| 178 | GLY | C | 4.14 | 54.53 | 27.06 | 10.17 |
| 178 | GLY | O | 3.77 | 55.70 | 27.02 | 10.19 |
| 179 | ALA | N | 4.12 | 53.80 | 28.17 | 10.24 |
| 179 | ALA | CA | 3.64 | 54.37 | 29.43 | 10.29 |
| 179 | ALA | CB | 3.62 | 53.30 | 30.51 | 10.41 |
| 179 | ALA | C | 4.50 | 55.55 | 29.87 | 10.30 |
| 179 | ALA | O | 3.98 | 56.55 | 30.37 | 10.27 |
| 180 | ALA | N | 5.81 | 55.43 | 29.68 | 10.36 |
| 180 | ALA | CA | 6.74 | 56.49 | 30.08 | 10.44 |
| 180 | ALA | CB | 8.18 | 56.00 | 29.97 | 10.60 |
| 180 | ALA | C | 6.55 | 57.75 | 29.23 | 10.50 |
| 180 | ALA | O | 6.51 | 58.86 | 29.76 | 10.43 |
| 181 | ILE | N | 6.45 | 57.57 | 27.92 | 10.61 |
| 181 | ILE | CA | 6.26 | 58.69 | 27.01 | 10.84 |
| 181 | ILE | CB | 6.26 | 58.20 | 25.54 | 10.83 |
| 181 | ILE | CG2 | 5.90 | 59.35 | 24.61 | 10.88 |
| 181 | ILE | CG1 | 7.63 | 57.62 | 25.20 | 10.88 |
| 181 | ILE | CD1 | 7.67 | 56.89 | 23.87 | 11.00 |
| 181 | ILE | C | 4.93 | 59.38 | 27.30 | 10.99 |
| 181 | ILE | O | 4.87 | 60.61 | 27.44 | 10.92 |
| 182 | GLN | N | 3.88 | 58.59 | 27.42 | 11.27 |
| 182 | GLN | CA | 2.54 | 59.09 | 27.69 | 11.66 |
| 182 | GLN | CB | 1.55 | 57.93 | 27.74 | 11.91 |
| 182 | GLN | CG | 0.10 | 58.35 | 27.81 | 12.34 |
| 182 | GLN | CD | −0.82 | 57.18 | 28.06 | 12.56 |
| 182 | GLN | OE1 | −0.53 | 56.05 | 27.66 | 12.64 |
| 182 | GLN | NE2 | −1.95 | 57.44 | 28.70 | 12.76 |
| 182 | GLN | C | 2.44 | 59.89 | 28.98 | 11.77 |
| 182 | GLN | O | 1.90 | 61.00 | 28.99 | 11.74 |
| 183 | GLU | N | 2.96 | 59.34 | 30.07 | 11.97 |
| 183 | GLU | CA | 2.90 | 60.03 | 31.36 | 12.17 |
| 183 | GLU | CB | 3.46 | 59.15 | 32.47 | 12.70 |
| 183 | GLU | CG | 3.54 | 59.80 | 33.85 | 13.57 |
| 183 | GLU | CD | 2.21 | 60.36 | 34.35 | 14.04 |
| 183 | GLU | OE1 | 1.15 | 59.81 | 33.97 | 14.62 |
| 183 | GLU | OE2 | 2.22 | 61.34 | 35.13 | 14.55 |
| 183 | GLU | C | 3.65 | 61.35 | 31.31 | 11.98 |
| 183 | GLU | O | 3.16 | 62.37 | 31.80 | 11.88 |
| 184 | TYR | N | 4.84 | 61.34 | 30.72 | 11.80 |
| 184 | TYR | CA | 5.63 | 62.55 | 30.66 | 11.69 |
| 184 | TYR | CB | 7.00 | 62.27 | 30.01 | 11.94 |
| 184 | TYR | CG | 7.93 | 63.45 | 30.10 | 12.23 |
| 184 | TYR | CD1 | 7.91 | 64.45 | 29.13 | 12.39 |
| 184 | TYR | CE1 | 8.70 | 65.59 | 29.25 | 12.67 |
| 184 | TYR | CD2 | 8.77 | 63.62 | 31.20 | 12.50 |
| 184 | TYR | CE2 | 9.57 | 64.75 | 31.33 | 12.71 |
| 184 | TYR | CZ | 9.52 | 65.73 | 30.35 | 12.77 |
| 184 | TYR | OH | 10.28 | 66.87 | 30.46 | 13.14 |
| 184 | TYR | C | 4.92 | 63.68 | 29.91 | 11.51 |
| 184 | TYR | O | 4.86 | 64.81 | 30.40 | 11.41 |
| 185 | ALA | N | 4.37 | 63.36 | 28.75 | 11.36 |
| 185 | ALA | CA | 3.68 | 64.36 | 27.94 | 11.36 |
| 185 | ALA | CB | 3.58 | 63.88 | 26.50 | 11.35 |
| 185 | ALA | C | 2.29 | 64.75 | 28.45 | 11.43 |
| 185 | ALA | O | 1.98 | 65.94 | 28.57 | 11.38 |
| 186 | GLU | N | 1.46 | 63.76 | 28.75 | 11.61 |
| 186 | GLU | CA | 0.10 | 64.06 | 29.22 | 11.84 |
| 186 | GLU | CB | −0.74 | 62.78 | 29.30 | 12.05 |
| 186 | GLU | CG | −0.91 | 62.08 | 27.95 | 12.39 |
| 186 | GLU | CD | −2.00 | 61.02 | 27.96 | 12.55 |
| 186 | GLU | OE1 | −2.29 | 60.46 | 29.05 | 12.75 |
| 186 | GLU | OE2 | −2.54 | 60.72 | 26.88 | 12.72 |
| 186 | GLU | C | 0.05 | 64.78 | 30.56 | 11.88 |
| 186 | GLU | O | −0.79 | 65.66 | 30.75 | 11.89 |
| 187 | SER | N | 0.95 | 64.44 | 31.48 | 11.93 |
| 187 | SER | CA | 0.94 | 65.09 | 32.79 | 12.05 |
| 187 | SER | CB | 1.95 | 64.43 | 33.73 | 12.11 |
| 187 | SER | OG | 3.28 | 64.66 | 33.30 | 12.15 |
| 187 | SER | C | 1.27 | 66.58 | 32.66 | 12.06 |
| 187 | SER | O | 0.96 | 67.37 | 33.55 | 12.13 |
| 188 | ARG | N | 1.88 | 66.95 | 31.54 | 12.04 |
| 188 | ARG | CA | 2.25 | 68.34 | 31.29 | 12.06 |
| 188 | ARG | CB | 3.65 | 68.41 | 30.69 | 12.05 |
| 188 | ARG | CG | 4.73 | 68.04 | 31.70 | 12.08 |
| 188 | ARG | CD | 6.10 | 67.88 | 31.06 | 12.07 |
| 188 | ARG | NE | 7.13 | 67.68 | 32.08 | 12.15 |
| 188 | ARG | CZ | 7.24 | 66.60 | 32.86 | 12.21 |
| 188 | ARG | NH1 | 6.37 | 65.61 | 32.73 | 12.25 |
| 188 | ARG | NH2 | 8.19 | 66.53 | 33.78 | 12.37 |
| 188 | ARG | C | 1.23 | 69.07 | 30.41 | 12.03 |
| 188 | ARG | O | 1.45 | 70.22 | 30.01 | 12.10 |
| 189 | GLY | N | 0.13 | 68.39 | 30.10 | 11.98 |
| 189 | GLY | CA | −0.93 | 69.00 | 29.30 | 11.94 |
| 189 | GLY | C | −0.90 | 68.81 | 27.80 | 11.84 |
| 189 | GLY | O | −1.67 | 69.45 | 27.08 | 12.10 |
| 190 | TYR | N | −0.02 | 67.94 | 27.31 | 11.60 |
| 190 | TYR | CA | 0.08 | 67.70 | 25.88 | 11.39 |
| 190 | TYR | CB | 1.55 | 67.57 | 25.46 | 11.40 |
| 190 | TYR | CG | 2.37 | 68.81 | 25.71 | 11.48 |
| 190 | TYR | CD1 | 2.89 | 69.09 | 26.98 | 11.57 |
| 190 | TYR | CE1 | 3.63 | 70.24 | 27.20 | 11.61 |
| 190 | TYR | CD2 | 2.62 | 69.71 | 24.68 | 11.52 |
| 190 | TYR | CE2 | 3.36 | 70.87 | 24.90 | 11.60 |
| 190 | TYR | CZ | 3.87 | 71.12 | 26.16 | 11.59 |
| 190 | TYR | OH | 4.60 | 72.27 | 26.38 | 11.85 |
| 190 | TYR | C | −0.68 | 66.46 | 25.42 | 11.25 |
| 190 | TYR | O | −1.01 | 65.58 | 26.22 | 11.23 |
| 191 | GLY | N | −0.94 | 66.40 | 24.12 | 11.10 |
| 191 | GLY | CA | −1.64 | 65.27 | 23.54 | 10.98 |
| 191 | GLY | C | −0.63 | 64.36 | 22.85 | 10.89 |
| 191 | GLY | O | 0.33 | 64.83 | 22.23 | 10.97 |
| 192 | VAL | N | −0.85 | 63.05 | 22.95 | 10.86 |
| 192 | VAL | CA | 0.04 | 62.08 | 22.34 | 10.88 |
| 192 | VAL | CB | 0.41 | 60.97 | 23.35 | 11.03 |
| 192 | VAL | CG1 | 1.33 | 59.95 | 22.71 | 11.18 |
| 192 | VAL | CG2 | 1.07 | 61.59 | 24.58 | 11.17 |
| 192 | VAL | C | −0.62 | 61.44 | 21.12 | 10.85 |
| 192 | VAL | O | −1.64 | 60.75 | 21.24 | 10.85 |
| 193 | VAL | N | −0.05 | 61.69 | 19.94 | 10.83 |
| 193 | VAL | CA | −0.59 | 61.12 | 18.71 | 10.98 |
| 193 | VAL | CB | 0.30 | 61.52 | 17.50 | 10.87 |
| 193 | VAL | CG1 | −0.08 | 60.72 | 16.27 | 10.97 |
| 193 | VAL | CG2 | 0.14 | 63.01 | 17.22 | 10.89 |
| 193 | VAL | C | −0.66 | 59.60 | 18.85 | 11.19 |
| 193 | VAL | O | 0.31 | 58.97 | 19.29 | 11.00 |
| 194 | ARG | N | −1.80 | 59.02 | 18.49 | 11.61 |
| 194 | ARG | CA | −2.00 | 57.58 | 18.61 | 12.18 |
| 194 | ARG | CB | −3.36 | 57.29 | 19.27 | 12.58 |
| 194 | ARG | CG | −3.58 | 57.92 | 20.63 | 13.24 |
| 194 | ARG | CD | −4.97 | 57.58 | 21.15 | 13.86 |
| 194 | ARG | NE | −5.26 | 58.22 | 22.43 | 14.45 |
| 194 | ARG | CZ | −6.41 | 58.08 | 23.08 | 14.72 |
| 194 | ARG | NH1 | −7.37 | 57.32 | 22.58 | 15.00 |
| 194 | ARG | NH2 | −6.60 | 58.70 | 24.24 | 15.03 |
| 194 | ARG | C | −1.96 | 56.82 | 17.29 | 12.31 |
| 194 | ARG | O | −1.62 | 55.63 | 17.27 | 12.39 |
| 195 | ASP | N | −2.31 | 57.49 | 16.20 | 12.48 |
| 195 | ASP | CA | −2.35 | 56.85 | 14.89 | 12.67 |
| 195 | ASP | CB | −3.08 | 57.74 | 13.90 | 13.13 |
| 195 | ASP | CG | −4.57 | 57.84 | 14.18 | 13.52 |
| 195 | ASP | OD1 | −5.04 | 57.21 | 15.15 | 13.95 |
| 195 | ASP | OD2 | −5.26 | 58.55 | 13.43 | 13.90 |
| 195 | ASP | C | −1.01 | 56.44 | 14.30 | 12.53 |
| 195 | ASP | O | −0.93 | 55.47 | 13.54 | 12.74 |
| 196 | LEU | N | 0.04 | 57.18 | 14.63 | 12.18 |
| 196 | LEU | CA | 1.38 | 56.89 | 14.11 | 11.86 |
| 196 | LEU | CB | 1.96 | 58.14 | 13.45 | 12.14 |
| 196 | LEU | CG | 1.06 | 58.70 | 12.34 | 12.45 |
| 196 | LEU | CD1 | 1.64 | 60.01 | 11.84 | 12.51 |
| 196 | LEU | CD2 | 0.95 | 57.69 | 11.20 | 12.67 |
| 196 | LEU | C | 2.25 | 56.40 | 15.26 | 11.50 |
| 196 | LEU | O | 2.16 | 56.90 | 16.38 | 11.39 |
| 197 | VAL | N | 3.11 | 55.42 | 14.97 | 11.17 |
| 197 | VAL | CA | 3.95 | 54.82 | 15.99 | 10.88 |
| 197 | VAL | CB | 3.34 | 53.46 | 16.43 | 10.96 |
| 197 | VAL | CG1 | 1.94 | 53.67 | 17.00 | 11.05 |
| 197 | VAL | CG2 | 3.29 | 52.51 | 15.24 | 11.10 |
| 197 | VAL | C | 5.40 | 54.56 | 15.60 | 10.64 |
| 197 | VAL | O | 5.78 | 54.68 | 14.43 | 10.65 |
| 198 | GLY | N | 6.20 | 54.21 | 16.61 | 10.42 |
| 198 | GLY | CA | 7.60 | 53.86 | 16.40 | 10.15 |
| 198 | GLY | C | 7.58 | 52.47 | 15.79 | 9.97 |
| 198 | GLY | O | 6.52 | 51.85 | 15.71 | 9.86 |
| 199 | HIS | N | 8.75 | 51.95 | 15.43 | 9.89 |
| 199 | HIS | CA | 8.78 | 50.66 | 14.75 | 9.85 |
| 199 | HIS | CB | 8.38 | 50.91 | 13.30 | 9.78 |
| 199 | HIS | CG | 9.20 | 52.00 | 12.66 | 9.67 |
| 199 | HIS | CD2 | 9.02 | 53.34 | 12.62 | 9.67 |
| 199 | HIS | ND1 | 10.43 | 51.77 | 12.09 | 9.61 |
| 199 | HIS | CE1 | 10.98 | 52.92 | 11.73 | 9.65 |
| 199 | HIS | NE2 | 10.14 | 53.89 | 12.04 | 9.74 |
| 199 | HIS | C | 10.15 | 50.02 | 14.75 | 9.88 |
| 199 | HIS | O | 11.15 | 50.65 | 15.10 | 9.80 |
| 200 | GLY | N | 10.19 | 48.75 | 14.34 | 10.04 |
| 200 | GLY | CA | 11.45 | 48.06 | 14.24 | 10.28 |
| 200 | GLY | C | 12.08 | 48.47 | 12.92 | 10.51 |
| 200 | GLY | O | 11.41 | 49.01 | 12.03 | 10.37 |
| 201 | VAL | N | 13.38 | 48.23 | 12.79 | 10.91 |
| 201 | VAL | CA | 14.12 | 48.55 | 11.57 | 11.46 |
| 201 | VAL | CB | 14.95 | 49.85 | 11.75 | 11.50 |
| 201 | VAL | CG1 | 15.76 | 50.14 | 10.49 | 11.57 |
| 201 | VAL | CG2 | 14.02 | 51.02 | 12.05 | 11.75 |
| 201 | VAL | C | 15.06 | 47.40 | 11.23 | 11.82 |
| 201 | VAL | O | 15.64 | 46.79 | 12.13 | 11.71 |
| 202 | GLY | N | 15.19 | 47.09 | 9.95 | 12.33 |
| 202 | GLY | CA | 16.06 | 46.02 | 9.51 | 13.15 |
| 202 | GLY | C | 15.29 | 44.72 | 9.31 | 13.77 |
| 202 | GLY | O | 14.71 | 44.20 | 10.26 | 13.73 |
| 203 | PRO | N | 15.29 | 44.16 | 8.09 | 14.29 |
| 203 | PRO | CD | 14.67 | 42.84 | 7.85 | 14.52 |
| 203 | PRO | CA | 15.96 | 44.64 | 6.87 | 14.74 |
| 203 | PRO | CB | 15.99 | 43.39 | 5.99 | 14.77 |
| 203 | PRO | CG | 14.70 | 42.73 | 6.33 | 14.70 |
| 203 | PRO | C | 15.33 | 45.84 | 6.15 | 15.06 |
| 203 | PRO | O | 15.97 | 46.45 | 5.30 | 15.24 |
| 204 | THR | N | 14.09 | 46.17 | 6.50 | 15.40 |
| 204 | THR | CA | 13.40 | 47.30 | 5.87 | 15.69 |
| 204 | THR | CB | 11.99 | 46.87 | 5.38 | 15.94 |
| 204 | THR | OG1 | 12.13 | 45.82 | 4.41 | 16.34 |
| 204 | THR | CG2 | 11.25 | 48.03 | 4.74 | 16.21 |
| 204 | THR | C | 13.27 | 48.46 | 6.85 | 15.61 |
| 204 | THR | O | 13.53 | 48.30 | 8.04 | 15.50 |
| 205 | MET | N | 12.86 | 49.63 | 6.36 | 15.62 |
| 205 | MET | CA | 12.70 | 50.80 | 7.22 | 15.65 |
| 205 | MET | CB | 12.57 | 52.09 | 6.39 | 15.70 |
| 205 | MET | CG | 11.24 | 52.28 | 5.66 | 15.78 |
| 205 | MET | SD | 11.08 | 53.93 | 4.89 | 15.74 |
| 205 | MET | CE | 12.21 | 53.78 | 3.53 | 15.97 |
| 205 | MET | C | 11.50 | 50.67 | 8.15 | 15.65 |
| 205 | MET | O | 11.34 | 51.45 | 9.08 | 15.67 |
| 206 | HIS | N | 10.65 | 49.67 | 7.89 | 15.66 |
| 206 | HIS | CA | 9.47 | 49.46 | 8.71 | 15.67 |
| 206 | HIS | CB | 8.22 | 49.98 | 7.99 | 16.09 |
| 206 | HIS | CG | 7.01 | 50.07 | 8.87 | 16.54 |
| 206 | HIS | CD2 | 5.96 | 49.23 | 9.03 | 16.71 |
| 206 | HIS | ND1 | 6.81 | 51.12 | 9.74 | 16.77 |
| 206 | HIS | CE1 | 5.68 | 50.92 | 10.40 | 16.83 |
| 206 | HIS | NE2 | 5.15 | 49.78 | 9.99 | 16.88 |
| 206 | HIS | C | 9.30 | 47.97 | 9.02 | 15.42 |
| 206 | HIS | O | 8.95 | 47.18 | 8.14 | 15.45 |
| 207 | GLU | N | 9.55 | 47.60 | 10.27 | 15.11 |
| 207 | GLU | CA | 9.45 | 46.21 | 10.69 | 14.87 |
| 207 | GLU | CB | 10.84 | 45.59 | 10.85 | 14.98 |
| 207 | GLU | CG | 11.71 | 45.64 | 9.61 | 15.25 |
| 207 | GLU | CD | 11.35 | 44.58 | 8.59 | 15.45 |
| 207 | GLU | OE1 | 11.94 | 44.59 | 7.50 | 15.60 |
| 207 | GLU | OE2 | 10.47 | 43.73 | 8.89 | 15.62 |
| 207 | GLU | C | 8.74 | 46.11 | 12.03 | 14.69 |
| 207 | GLU | O | 8.59 | 47.11 | 12.74 | 14.53 |
| 208 | GLU | N | 8.31 | 44.90 | 12.37 | 14.51 |
| 208 | GLU | CA | 7.69 | 44.66 | 13.65 | 14.38 |
| 208 | GLU | CB | 7.06 | 43.26 | 13.68 | 14.79 |
| 208 | GLU | CG | 5.73 | 43.19 | 12.98 | 15.50 |
| 208 | GLU | CD | 4.63 | 43.85 | 13.78 | 15.84 |
| 208 | GLU | OE1 | 3.69 | 44.41 | 13.17 | 16.25 |
| 208 | GLU | OE2 | 4.70 | 43.80 | 15.03 | 16.23 |
| 208 | GLU | C | 8.87 | 44.72 | 14.62 | 14.00 |
| 208 | GLU | O | 10.02 | 44.53 | 14.21 | 13.96 |
| 209 | PRO | N | 8.62 | 45.02 | 15.90 | 13.63 |
| 209 | PRO | CD | 9.69 | 44.96 | 16.92 | 13.61 |
| 209 | PRO | CA | 7.33 | 45.30 | 16.52 | 13.28 |
| 209 | PRO | CB | 7.57 | 44.87 | 17.96 | 13.44 |
| 209 | PRO | CG | 8.96 | 45.38 | 18.19 | 13.51 |
| 209 | PRO | C | 7.00 | 46.78 | 16.44 | 12.90 |
| 209 | PRO | O | 7.89 | 47.61 | 16.22 | 12.79 |
| 210 | MET | N | 5.73 | 47.11 | 16.60 | 12.51 |
| 210 | MET | CA | 5.34 | 48.51 | 16.59 | 12.16 |
| 210 | MET | CB | 3.84 | 48.66 | 16.32 | 12.69 |
| 210 | MET | CG | 3.44 | 48.27 | 14.91 | 13.41 |
| 210 | MET | SD | 4.33 | 49.20 | 13.63 | 14.20 |
| 210 | MET | CE | 5.65 | 48.05 | 13.21 | 14.10 |
| 210 | MET | C | 5.68 | 49.03 | 17.99 | 11.58 |
| 210 | MET | O | 5.68 | 48.27 | 18.96 | 11.41 |
| 211 | VAL | N | 5.99 | 50.31 | 18.07 | 11.04 |
| 211 | VAL | CA | 6.35 | 50.93 | 19.34 | 10.66 |
| 211 | VAL | CB | 7.85 | 51.33 | 19.35 | 10.64 |
| 211 | VAL | CG1 | 8.25 | 51.81 | 20.73 | 10.67 |
| 211 | VAL | CG2 | 8.72 | 50.15 | 18.93 | 10.72 |
| 211 | VAL | C | 5.49 | 52.16 | 19.56 | 10.41 |
| 211 | VAL | O | 5.90 | 53.29 | 19.29 | 10.18 |
| 212 | PRO | N | 4.25 | 51.96 | 20.05 | 10.26 |
| 212 | PRO | CD | 3.59 | 50.67 | 20.32 | 10.27 |
| 212 | PRO | CA | 3.34 | 53.07 | 20.31 | 10.27 |
| 212 | PRO | CB | 2.08 | 52.38 | 20.82 | 10.32 |
| 212 | PRO | CG | 2.14 | 51.03 | 20.18 | 10.34 |
| 212 | PRO | C | 3.90 | 54.05 | 21.34 | 10.23 |
| 212 | PRO | O | 4.66 | 53.66 | 22.22 | 10.11 |
| 213 | ASN | N | 3.49 | 55.31 | 21.23 | 10.26 |
| 213 | ASN | CA | 3.95 | 56.34 | 22.15 | 10.49 |
| 213 | ASN | CB | 4.09 | 57.66 | 21.40 | 10.45 |
| 213 | ASN | CG | 5.20 | 57.61 | 20.39 | 10.49 |
| 213 | ASN | OD1 | 6.36 | 57.44 | 20.74 | 10.48 |
| 213 | ASN | ND2 | 4.85 | 57.74 | 19.11 | 10.65 |
| 213 | ASN | C | 3.05 | 56.48 | 23.36 | 10.74 |
| 213 | ASN | O | 3.17 | 57.42 | 24.14 | 10.71 |
| 214 | TYR | N | 2.14 | 55.52 | 23.51 | 11.14 |
| 214 | TYR | CA | 1.23 | 55.46 | 24.64 | 11.58 |
| 214 | TYR | CB | −0.14 | 56.05 | 24.30 | 12.28 |
| 214 | TYR | CG | −0.90 | 55.29 | 23.24 | 13.02 |
| 214 | TYR | CD1 | −0.58 | 55.43 | 21.89 | 13.29 |
| 214 | TYR | CE1 | −1.26 | 54.72 | 20.91 | 13.72 |
| 214 | TYR | CD2 | −1.92 | 54.41 | 23.59 | 13.44 |
| 214 | TYR | CE2 | −2.60 | 53.68 | 22.62 | 13.82 |
| 214 | TYR | CZ | −2.27 | 53.84 | 21.29 | 13.87 |
| 214 | TYR | OH | −2.95 | 53.12 | 20.33 | 14.36 |
| 214 | TYR | C | 1.11 | 53.97 | 24.95 | 11.57 |
| 214 | TYR | O | 1.24 | 53.13 | 24.05 | 11.41 |
| 215 | GLY | N | 0.87 | 53.64 | 26.21 | 11.67 |
| 215 | GLY | CA | 0.75 | 52.24 | 26.57 | 11.86 |
| 215 | GLY | C | 0.66 | 52.03 | 28.06 | 11.99 |
| 215 | GLY | O | 0.59 | 52.98 | 28.83 | 11.95 |
| 216 | ILE | N | 0.67 | 50.76 | 28.45 | 12.15 |
| 216 | ILE | CA | 0.56 | 50.38 | 29.86 | 12.36 |
| 216 | ILE | CB | −0.67 | 49.48 | 30.08 | 12.57 |
| 216 | ILE | CG2 | −0.76 | 49.06 | 31.54 | 12.64 |
| 216 | ILE | CG1 | −1.93 | 50.23 | 29.85 | 12.79 |
| 216 | ILE | CD1 | −3.20 | 49.42 | 29.78 | 13.03 |
| 216 | ILE | C | 1.81 | 49.63 | 30.31 | 12.35 |
| 216 | ILE | O | 2.25 | 48.69 | 29.65 | 12.22 |
| 217 | ALA | N | 2.37 | 50.06 | 31.43 | 12.42 |
| 217 | ALA | CA | 3.56 | 49.44 | 31.97 | 12.50 |
| 217 | ALA | CB | 3.98 | 50.14 | 33.26 | 12.63 |
| 217 | ALA | C | 3.35 | 47.95 | 32.22 | 12.51 |
| 217 | ALA | O | 2.27 | 47.52 | 32.63 | 12.53 |
| 218 | GLY | N | 4.39 | 47.17 | 31.95 | 12.51 |
| 218 | GLY | CA | 4.33 | 45.74 | 32.17 | 12.54 |
| 218 | GLY | C | 3.56 | 44.94 | 31.13 | 12.48 |
| 218 | GLY | O | 3.37 | 43.74 | 31.31 | 12.62 |
| 219 | ARG | N | 3.14 | 45.58 | 30.05 | 12.42 |
| 219 | ARG | CA | 2.39 | 44.89 | 29.01 | 12.26 |
| 219 | ARG | CB | 0.95 | 45.42 | 28.95 | 12.39 |
| 219 | ARG | CG | 0.21 | 45.30 | 30.27 | 12.41 |
| 219 | ARG | CD | −1.27 | 45.56 | 30.10 | 12.45 |
| 219 | ARG | NE | −1.98 | 45.47 | 31.37 | 12.47 |
| 219 | ARG | CZ | −3.30 | 45.45 | 31.49 | 12.44 |
| 219 | ARG | NH1 | −4.07 | 45.52 | 30.41 | 12.39 |
| 219 | ARG | NH2 | −3.85 | 45.36 | 32.69 | 12.61 |
| 219 | ARG | C | 3.05 | 45.02 | 27.64 | 12.14 |
| 219 | ARG | O | 3.92 | 45.86 | 27.44 | 12.00 |
| 220 | GLY | N | 2.62 | 44.18 | 26.71 | 12.05 |
| 220 | GLY | CA | 3.18 | 44.22 | 25.37 | 12.06 |
| 220 | GLY | C | 4.33 | 43.26 | 25.18 | 12.00 |
| 220 | GLY | O | 4.73 | 42.56 | 26.12 | 11.91 |
| 221 | LEU | N | 4.88 | 43.23 | 23.97 | 12.07 |
| 221 | LEU | CA | 5.98 | 42.33 | 23.67 | 12.15 |
| 221 | LEU | CB | 6.37 | 42.46 | 22.19 | 12.49 |
| 221 | LEU | CG | 5.43 | 41.82 | 21.16 | 12.88 |
| 221 | LEU | CD1 | 5.87 | 42.20 | 19.77 | 13.17 |
| 221 | LEU | CD2 | 5.43 | 40.31 | 21.33 | 13.05 |
| 221 | LEU | C | 7.21 | 42.56 | 24.53 | 12.12 |
| 221 | LEU | O | 7.57 | 43.70 | 24.84 | 11.86 |
| 222 | ARG | N | 7.85 | 41.47 | 24.93 | 12.26 |
| 222 | ARG | CA | 9.05 | 41.54 | 25.75 | 12.53 |
| 222 | ARG | CB | 9.31 | 40.19 | 26.43 | 13.02 |
| 222 | ARG | CG | 8.26 | 39.83 | 27.48 | 13.83 |
| 222 | ARG | CD | 8.41 | 38.40 | 27.98 | 14.49 |
| 222 | ARG | NE | 7.33 | 38.01 | 28.89 | 15.10 |
| 222 | ARG | CZ | 7.22 | 38.41 | 30.15 | 15.38 |
| 222 | ARG | NH1 | 8.13 | 39.22 | 30.67 | 15.69 |
| 222 | ARG | NH2 | 6.20 | 38.01 | 30.90 | 15.64 |
| 222 | ARG | C | 10.24 | 41.95 | 24.88 | 12.43 |
| 222 | ARG | O | 10.43 | 41.42 | 23.77 | 12.33 |
| 223 | LEU | N | 11.03 | 42.89 | 25.37 | 12.39 |
| 223 | LEU | CA | 12.19 | 43.38 | 24.64 | 12.53 |
| 223 | LEU | CB | 12.60 | 44.76 | 25.17 | 12.43 |
| 223 | LEU | CG | 11.46 | 45.79 | 25.14 | 12.45 |
| 223 | LEU | CD1 | 11.95 | 47.10 | 25.74 | 12.44 |
| 223 | LEU | CD2 | 10.99 | 46.00 | 23.71 | 12.52 |
| 223 | LEU | C | 13.35 | 42.41 | 24.75 | 12.71 |
| 223 | LEU | O | 13.63 | 41.88 | 25.83 | 12.78 |
| 224 | ARG | N | 14.02 | 42.17 | 23.63 | 12.93 |
| 224 | ARG | CA | 15.14 | 41.24 | 23.58 | 13.26 |
| 224 | ARG | CB | 14.79 | 40.03 | 22.71 | 13.61 |
| 224 | ARG | CG | 13.56 | 39.26 | 23.15 | 14.08 |
| 224 | ARG | CD | 13.73 | 38.66 | 24.53 | 14.61 |
| 224 | ARG | NE | 12.57 | 37.86 | 24.91 | 15.11 |
| 224 | ARG | CZ | 12.34 | 37.40 | 26.13 | 15.29 |
| 224 | ARG | NH1 | 13.19 | 37.65 | 27.11 | 15.55 |
| 224 | ARG | NH2 | 11.25 | 36.69 | 26.38 | 15.53 |
| 224 | ARG | C | 16.40 | 41.88 | 23.02 | 13.30 |
| 224 | ARG | O | 16.34 | 42.80 | 22.20 | 13.18 |
| 225 | GLU | N | 17.54 | 41.37 | 23.47 | 13.45 |
| 225 | GLU | CA | 18.84 | 41.85 | 23.02 | 13.56 |
| 225 | GLU | CB | 19.95 | 41.04 | 23.70 | 14.11 |
| 225 | GLU | CG | 21.36 | 41.49 | 23.38 | 14.98 |
| 225 | GLU | CD | 22.40 | 40.56 | 23.98 | 15.45 |
| 225 | GLU | OE1 | 22.25 | 40.19 | 25.16 | 15.92 |
| 225 | GLU | OE2 | 23.37 | 40.22 | 23.28 | 15.91 |
| 225 | GLU | C | 18.90 | 41.63 | 21.51 | 13.28 |
| 225 | GLU | O | 18.56 | 40.56 | 21.02 | 13.32 |
| 226 | GLY | N | 19.32 | 42.66 | 20.77 | 13.02 |
| 226 | GLY | CA | 19.41 | 42.52 | 19.33 | 12.69 |
| 226 | GLY | C | 18.29 | 43.22 | 18.59 | 12.41 |
| 226 | GLY | O | 18.36 | 43.40 | 17.38 | 12.37 |
| 227 | MET | N | 17.23 | 43.60 | 19.30 | 12.13 |
| 227 | MET | CA | 16.14 | 44.31 | 18.66 | 11.89 |
| 227 | MET | CB | 14.92 | 44.43 | 19.59 | 12.17 |
| 227 | MET | CG | 14.12 | 43.16 | 19.78 | 12.51 |
| 227 | MET | SD | 12.67 | 43.49 | 20.79 | 12.81 |
| 227 | MET | CE | 11.39 | 43.52 | 19.53 | 13.34 |
| 227 | MET | C | 16.60 | 45.72 | 18.30 | 11.57 |
| 227 | MET | O | 17.30 | 46.36 | 19.09 | 11.51 |
| 228 | VAL | N | 16.22 | 46.18 | 17.12 | 11.26 |
| 228 | VAL | CA | 16.55 | 47.53 | 16.68 | 11.01 |
| 228 | VAL | CB | 17.35 | 47.53 | 15.36 | 11.02 |
| 228 | VAL | CG1 | 17.53 | 48.96 | 14.87 | 11.04 |
| 228 | VAL | CG2 | 18.70 | 46.88 | 15.57 | 11.13 |
| 228 | VAL | C | 15.21 | 48.22 | 16.48 | 10.80 |
| 228 | VAL | O | 14.39 | 47.79 | 15.67 | 10.74 |
| 229 | LEU | N | 15.00 | 49.29 | 17.24 | 10.64 |
| 229 | LEU | CA | 13.74 | 50.01 | 17.20 | 10.52 |
| 229 | LEU | CB | 12.96 | 49.78 | 18.49 | 10.82 |
| 229 | LEU | CG | 12.81 | 48.36 | 19.04 | 11.04 |
| 229 | LEU | CD1 | 12.18 | 48.41 | 20.43 | 11.28 |
| 229 | LEU | CD2 | 11.96 | 47.55 | 18.09 | 11.24 |
| 229 | LEU | C | 13.94 | 51.51 | 17.09 | 10.44 |
| 229 | LEU | O | 15.05 | 52.02 | 17.22 | 10.24 |
| 230 | THR | N | 12.83 | 52.19 | 16.84 | 10.41 |
| 230 | THR | CA | 12.84 | 53.64 | 16.84 | 10.45 |
| 230 | THR | CB | 12.41 | 54.28 | 15.51 | 10.69 |
| 230 | THR | OG1 | 11.03 | 53.98 | 15.26 | 10.96 |
| 230 | THR | CG2 | 13.28 | 53.80 | 14.37 | 10.91 |
| 230 | THR | C | 11.79 | 54.02 | 17.86 | 10.25 |
| 230 | THR | O | 10.81 | 53.30 | 18.10 | 10.48 |
| 231 | ILE | N | 12.03 | 55.14 | 18.52 | 9.90 |
| 231 | ILE | CA | 11.09 | 55.72 | 19.45 | 9.60 |
| 231 | ILE | CB | 11.62 | 55.68 | 20.90 | 9.54 |
| 231 | ILE | CG2 | 10.89 | 56.70 | 21.76 | 9.63 |
| 231 | ILE | CG1 | 11.40 | 54.26 | 21.45 | 9.58 |
| 231 | ILE | CD1 | 11.97 | 54.02 | 22.82 | 9.58 |
| 231 | ILE | C | 11.00 | 57.12 | 18.87 | 9.51 |
| 231 | ILE | O | 11.99 | 57.84 | 18.77 | 9.27 |
| 232 | GLU | N | 9.79 | 57.47 | 18.44 | 9.51 |
| 232 | GLU | CA | 9.56 | 58.74 | 17.77 | 9.66 |
| 232 | GLU | CB | 9.59 | 58.50 | 16.26 | 9.80 |
| 232 | GLU | CG | 8.67 | 57.39 | 15.76 | 10.16 |
| 232 | GLU | CD | 8.88 | 57.11 | 14.29 | 10.20 |
| 232 | GLU | OE1 | 8.56 | 58.00 | 13.47 | 10.68 |
| 232 | GLU | OE2 | 9.39 | 56.02 | 13.96 | 10.38 |
| 232 | GLU | C | 8.26 | 59.42 | 18.15 | 9.62 |
| 232 | GLU | O | 7.36 | 59.57 | 17.33 | 9.71 |
| 233 | PRO | N | 8.16 | 59.87 | 19.40 | 9.67 |
| 233 | PRO | CD | 9.17 | 59.88 | 20.47 | 9.83 |
| 233 | PRO | CA | 6.93 | 60.53 | 19.83 | 9.65 |
| 233 | PRO | CB | 7.20 | 60.83 | 21.31 | 9.86 |
| 233 | PRO | CG | 8.69 | 61.00 | 21.35 | 10.03 |
| 233 | PRO | C | 6.54 | 61.78 | 19.06 | 9.57 |
| 233 | PRO | O | 7.37 | 62.64 | 18.76 | 9.47 |
| 234 | MET | N | 5.25 | 61.85 | 18.73 | 9.64 |
| 234 | MET | CA | 4.64 | 62.99 | 18.06 | 9.70 |
| 234 | MET | CB | 3.87 | 62.55 | 16.82 | 9.97 |
| 234 | MET | CG | 4.79 | 62.21 | 15.66 | 10.20 |
| 234 | MET | SD | 4.12 | 60.97 | 14.56 | 10.39 |
| 234 | MET | CE | 4.43 | 59.49 | 15.54 | 10.56 |
| 234 | MET | C | 3.72 | 63.54 | 19.14 | 9.71 |
| 234 | MET | O | 2.75 | 62.88 | 19.55 | 9.63 |
| 235 | ILE | N | 4.05 | 64.73 | 19.61 | 9.74 |
| 235 | ILE | CA | 3.33 | 65.39 | 20.68 | 9.89 |
| 235 | ILE | CB | 4.33 | 65.72 | 21.82 | 9.97 |
| 235 | ILE | CG2 | 3.62 | 66.40 | 22.98 | 10.11 |
| 235 | ILE | CG1 | 5.06 | 64.45 | 22.27 | 10.04 |
| 235 | ILE | CD1 | 4.15 | 63.34 | 22.80 | 10.07 |
| 235 | ILE | C | 2.66 | 66.66 | 20.19 | 9.98 |
| 235 | ILE | O | 3.28 | 67.47 | 19.49 | 9.80 |
| 236 | ASN | N | 1.38 | 66.82 | 20.54 | 10.22 |
| 236 | ASN | CA | 0.62 | 67.99 | 20.12 | 10.56 |
| 236 | ASN | CB | −0.72 | 67.58 | 19.49 | 10.58 |
| 236 | ASN | CG | −0.56 | 66.66 | 18.30 | 10.55 |
| 236 | ASN | OD1 | 0.52 | 66.57 | 17.70 | 10.58 |
| 236 | ASN | ND2 | −1.64 | 65.97 | 17.93 | 10.68 |
| 236 | ASN | C | 0.32 | 68.89 | 21.32 | 10.83 |
| 236 | ASN | O | 0.22 | 68.42 | 22.45 | 10.84 |
| 237 | THR | N | 0.16 | 70.19 | 21.07 | 11.21 |
| 237 | THR | CA | −0.14 | 71.10 | 22.17 | 11.70 |
| 237 | THR | CB | 0.03 | 72.58 | 21.74 | 11.76 |
| 237 | THR | OG1 | −0.79 | 72.84 | 20.60 | 11.80 |
| 237 | THR | CG2 | 1.48 | 72.87 | 21.41 | 11.88 |
| 237 | THR | C | −1.56 | 70.89 | 22.68 | 12.00 |
| 237 | THR | O | −1.85 | 71.16 | 23.84 | 12.10 |
| 238 | GLY | N | −2.44 | 70.41 | 21.81 | 12.30 |
| 238 | GLY | CA | −3.83 | 70.19 | 22.20 | 12.73 |
| 238 | GLY | C | −4.31 | 68.76 | 22.02 | 13.01 |
| 238 | GLY | O | −3.78 | 67.83 | 22.63 | 13.10 |
| 239 | ASP | N | −5.31 | 68.58 | 21.16 | 13.30 |
| 239 | ASP | CA | −5.88 | 67.27 | 20.90 | 13.66 |
| 239 | ASP | CB | −7.15 | 67.40 | 20.05 | 14.30 |
| 239 | ASP | CG | −8.24 | 68.19 | 20.75 | 14.85 |
| 239 | ASP | OD1 | −8.61 | 67.84 | 21.88 | 15.33 |
| 239 | ASP | OD2 | −8.74 | 69.18 | 20.15 | 15.40 |
| 239 | ASP | C | −4.89 | 66.33 | 20.21 | 13.63 |
| 239 | ASP | O | −4.03 | 66.77 | 19.45 | 13.47 |
| 240 | TRP | N | −5.04 | 65.03 | 20.46 | 13.71 |
| 240 | TRP | CA | −4.15 | 64.03 | 19.88 | 13.91 |
| 240 | TRP | CB | −4.08 | 62.79 | 20.78 | 14.27 |
| 240 | TRP | CG | −5.40 | 62.14 | 21.06 | 14.70 |
| 240 | TRP | CD2 | −6.05 | 61.14 | 20.27 | 14.90 |
| 240 | TRP | CE2 | −7.27 | 60.82 | 20.91 | 15.03 |
| 240 | TRP | CE3 | −5.72 | 60.47 | 19.08 | 15.02 |
| 240 | TRP | CD1 | −6.23 | 62.38 | 22.12 | 14.87 |
| 240 | TRP | NE1 | −7.35 | 61.59 | 22.04 | 15.05 |
| 240 | TRP | CZ2 | −8.15 | 59.86 | 20.40 | 15.13 |
| 240 | TRP | CZ3 | −6.60 | 59.52 | 18.57 | 15.10 |
| 240 | TRP | CH2 | −7.80 | 59.23 | 19.24 | 15.15 |
| 240 | TRP | C | −4.53 | 63.58 | 18.46 | 13.89 |
| 240 | TRP | O | −3.71 | 62.99 | 17.76 | 13.71 |
| 241 | GLU | N | −5.76 | 63.86 | 18.05 | 13.96 |
| 241 | GLU | CA | −6.23 | 63.45 | 16.73 | 14.15 |
| 241 | GLU | CB | −7.75 | 63.61 | 16.63 | 14.47 |
| 241 | GLU | CG | −8.52 | 62.71 | 17.59 | 15.07 |
| 241 | GLU | CD | −8.92 | 63.42 | 18.88 | 15.43 |
| 241 | GLU | OE1 | −8.17 | 64.31 | 19.34 | 15.74 |
| 241 | GLU | OE2 | −9.98 | 63.08 | 19.43 | 15.80 |
| 241 | GLU | C | −5.55 | 64.17 | 15.56 | 14.10 |
| 241 | GLU | O | −5.24 | 65.36 | 15.65 | 13.92 |
| 242 | ILE | N | −5.35 | 63.44 | 14.47 | 14.19 |
| 242 | ILE | CA | −4.71 | 63.98 | 13.27 | 14.42 |
| 242 | ILE | CB | −3.26 | 63.45 | 13.11 | 14.27 |
| 242 | ILE | CG2 | −2.45 | 63.78 | 14.35 | 14.21 |
| 242 | ILE | CG1 | −3.27 | 61.94 | 12.87 | 14.26 |
| 242 | ILE | CD1 | −1.90 | 61.36 | 12.52 | 14.25 |
| 242 | ILE | C | −5.50 | 63.61 | 12.01 | 14.72 |
| 242 | ILE | O | −6.37 | 62.74 | 12.04 | 14.69 |
| 243 | ASP | N | −5.18 | 64.29 | 10.91 | 15.15 |
| 243 | ASP | CA | −5.84 | 64.04 | 9.63 | 15.68 |
| 243 | ASP | CB | −6.64 | 65.27 | 9.18 | 16.05 |
| 243 | ASP | CG | −5.76 | 66.43 | 8.75 | 16.27 |
| 243 | ASP | OD1 | −4.52 | 66.31 | 8.81 | 16.61 |
| 243 | ASP | OD2 | −6.32 | 67.47 | 8.34 | 16.60 |
| 243 | ASP | C | −4.78 | 63.72 | 8.57 | 15.86 |
| 243 | ASP | O | −3.59 | 63.68 | 8.88 | 15.92 |
| 244 | THR | N | −5.21 | 63.50 | 7.34 | 16.13 |
| 244 | THR | CA | −4.29 | 63.20 | 6.25 | 16.47 |
| 244 | THR | CB | −4.34 | 61.72 | 5.84 | 16.53 |
| 244 | THR | OG1 | −4.02 | 60.90 | 6.97 | 16.58 |
| 244 | THR | CG2 | −3.33 | 61.43 | 4.73 | 16.61 |
| 244 | THR | C | −4.62 | 64.05 | 5.03 | 16.68 |
| 244 | THR | O | −5.79 | 64.24 | 4.69 | 16.72 |
| 245 | ASP | N | −3.58 | 64.55 | 4.37 | 16.94 |
| 245 | ASP | CA | −3.73 | 65.38 | 3.18 | 17.25 |
| 245 | ASP | CB | −2.35 | 65.66 | 2.59 | 17.38 |
| 245 | ASP | CG | −2.37 | 66.72 | 1.51 | 17.53 |
| 245 | ASP | OD1 | −3.20 | 66.61 | 0.58 | 17.71 |
| 245 | ASP | OD2 | −1.55 | 67.66 | 1.58 | 17.64 |
| 245 | ASP | C | −4.59 | 64.64 | 2.15 | 17.40 |
| 245 | ASP | O | −4.24 | 63.55 | 1.70 | 17.39 |
| 246 | MET | N | −5.71 | 65.25 | 1.77 | 17.61 |
| 246 | MET | CA | −6.61 | 64.64 | 0.80 | 17.79 |
| 246 | MET | CB | −8.01 | 65.23 | 0.92 | 18.44 |
| 246 | MET | CG | −8.69 | 64.91 | 2.24 | 19.24 |
| 246 | MET | SD | −8.81 | 63.13 | 2.51 | 20.36 |
| 246 | MET | CE | −10.32 | 62.76 | 1.63 | 20.14 |
| 246 | MET | C | −6.12 | 64.79 | −0.64 | 17.51 |
| 246 | MET | O | −6.62 | 64.12 | −1.55 | 17.50 |
| 247 | LYS | N | −5.14 | 65.66 | −0.84 | 17.14 |
| 247 | LYS | CA | −4.60 | 65.91 | −2.17 | 16.71 |
| 247 | LYS | CB | −4.14 | 67.36 | −2.27 | 17.09 |
| 247 | LYS | CG | −3.71 | 67.79 | −3.66 | 17.54 |
| 247 | LYS | CD | −3.26 | 69.25 | −3.68 | 17.91 |
| 247 | LYS | CE | −4.34 | 70.17 | −3.13 | 18.12 |
| 247 | LYS | NZ | −5.62 | 70.06 | −3.88 | 18.39 |
| 247 | LYS | C | −3.44 | 64.98 | −2.52 | 16.15 |
| 247 | LYS | O | −3.30 | 64.56 | −3.67 | 16.21 |
| 248 | THR | N | −2.63 | 64.64 | −1.53 | 15.42 |
| 248 | THR | CA | −1.46 | 63.79 | −1.75 | 14.68 |
| 248 | THR | CB | −0.17 | 64.50 | −1.34 | 14.73 |
| 248 | THR | OG1 | −0.15 | 64.66 | 0.08 | 14.63 |
| 248 | THR | CG2 | −0.09 | 65.88 | −1.99 | 14.74 |
| 248 | THR | C | −1.47 | 62.47 | −0.98 | 14.10 |
| 248 | THR | O | −0.79 | 61.52 | −1.36 | 13.96 |
| 249 | GLY | N | −2.23 | 62.43 | 0.11 | 13.50 |
| 249 | GLY | CA | −2.29 | 61.23 | 0.93 | 12.80 |
| 249 | GLY | C | −1.07 | 61.08 | 1.83 | 12.29 |
| 249 | GLY | O | −0.89 | 60.04 | 2.46 | 12.16 |
| 250 | TRP | N | −0.24 | 62.12 | 1.90 | 11.88 |
| 250 | TRP | CA | 0.96 | 62.04 | 2.72 | 11.58 |
| 250 | TRP | CB | 2.19 | 62.37 | 1.86 | 11.46 |
| 250 | TRP | CG | 2.56 | 61.23 | 0.96 | 11.24 |
| 250 | TRP | CD2 | 3.09 | 59.97 | 1.38 | 11.18 |
| 250 | TRP | CE2 | 3.24 | 59.17 | 0.23 | 11.15 |
| 250 | TRP | CE3 | 3.46 | 59.43 | 2.62 | 11.24 |
| 250 | TRP | CD1 | 2.42 | 61.15 | −0.39 | 11.22 |
| 250 | TRP | NE1 | 2.82 | 59.92 | −0.84 | 11.15 |
| 250 | TRP | CZ2 | 3.73 | 57.86 | 0.28 | 11.17 |
| 250 | TRP | CZ3 | 3.95 | 58.14 | 2.68 | 11.35 |
| 250 | TRP | CH2 | 4.08 | 57.36 | 1.51 | 11.34 |
| 250 | TRP | C | 0.97 | 62.86 | 4.01 | 11.48 |
| 250 | TRP | O | 0.93 | 62.29 | 5.10 | 11.40 |
| 251 | ALA | N | 1.01 | 64.18 | 3.91 | 11.46 |
| 251 | ALA | CA | 1.07 | 65.02 | 5.10 | 11.51 |
| 251 | ALA | CB | 1.13 | 66.49 | 4.70 | 11.53 |
| 251 | ALA | C | −0.06 | 64.83 | 6.12 | 11.55 |
| 251 | ALA | O | −1.23 | 64.72 | 5.75 | 11.58 |
| 252 | HIS | N | 0.32 | 64.79 | 7.39 | 11.67 |
| 252 | HIS | CA | −0.64 | 64.66 | 8.48 | 11.85 |
| 252 | HIS | CB | −0.27 | 63.51 | 9.41 | 12.33 |
| 252 | HIS | CG | −0.25 | 62.18 | 8.73 | 12.69 |
| 252 | HIS | CD2 | 0.76 | 61.32 | 8.48 | 12.88 |
| 252 | HIS | ND1 | −1.39 | 61.62 | 8.18 | 12.96 |
| 252 | HIS | CE1 | −1.07 | 60.47 | 7.62 | 13.03 |
| 252 | HIS | NE2 | 0.23 | 60.26 | 7.78 | 12.92 |
| 252 | HIS | C | −0.58 | 65.96 | 9.27 | 11.75 |
| 252 | HIS | O | 0.50 | 66.50 | 9.51 | 11.53 |
| 253 | LYS | N | −1.75 | 66.45 | 9.68 | 11.73 |
| 253 | LYS | CA | −1.84 | 67.68 | 10.46 | 11.76 |
| 253 | LYS | CB | −2.44 | 68.82 | 9.63 | 12.32 |
| 253 | LYS | CG | −1.59 | 69.30 | 8.47 | 12.96 |
| 253 | LYS | CD | −2.21 | 70.54 | 7.85 | 13.57 |
| 253 | LYS | CE | −1.34 | 71.13 | 6.75 | 13.96 |
| 253 | LYS | NZ | −1.93 | 72.39 | 6.22 | 14.33 |
| 253 | LYS | C | −2.72 | 67.44 | 11.68 | 11.48 |
| 253 | LYS | O | −3.53 | 66.51 | 11.70 | 11.36 |
| 254 | THR | N | −2.57 | 68.29 | 12.69 | 11.21 |
| 254 | THR | CA | −3.40 | 68.18 | 13.88 | 11.08 |
| 254 | THR | CB | −2.93 | 69.14 | 14.98 | 10.94 |
| 254 | THR | OG1 | −2.95 | 70.48 | 14.48 | 10.63 |
| 254 | THR | CG2 | −1.53 | 68.79 | 15.44 | 10.82 |
| 254 | THR | C | −4.82 | 68.56 | 13.48 | 11.20 |
| 254 | THR | O | −5.03 | 69.45 | 12.66 | 11.19 |
| 255 | ILE | N | −5.80 | 67.88 | 14.06 | 11.42 |
| 255 | ILE | CA | −7.19 | 68.19 | 13.77 | 11.75 |
| 255 | ILE | CB | −8.14 | 67.12 | 14.36 | 12.05 |
| 255 | ILE | CG2 | −9.57 | 67.65 | 14.42 | 12.34 |
| 255 | ILE | CG1 | −8.06 | 65.83 | 13.53 | 12.30 |
| 255 | ILE | CD1 | −8.59 | 65.98 | 12.12 | 12.62 |
| 255 | ILE | C | −7.55 | 69.55 | 14.36 | 11.75 |
| 255 | ILE | O | −8.30 | 70.31 | 13.75 | 11.86 |
| 256 | ASP | N | −7.01 | 69.87 | 15.53 | 11.73 |
| 256 | ASP | CA | −7.34 | 71.14 | 16.17 | 11.78 |
| 256 | ASP | CB | −7.27 | 71.00 | 17.71 | 12.21 |
| 256 | ASP | CG | −5.85 | 70.90 | 18.24 | 12.55 |
| 256 | ASP | OD1 | −4.89 | 70.87 | 17.45 | 12.69 |
| 256 | ASP | OD2 | −5.70 | 70.85 | 19.48 | 13.00 |
| 256 | ASP | C | −6.57 | 72.37 | 15.71 | 11.58 |
| 256 | ASP | O | −6.89 | 73.49 | 16.11 | 11.42 |
| 257 | GLY | N | −5.55 | 72.17 | 14.87 | 11.40 |
| 257 | GLY | CA | −4.79 | 73.30 | 14.38 | 11.48 |
| 257 | GLY | C | −3.62 | 73.70 | 15.26 | 11.42 |
| 257 | GLY | O | −2.86 | 74.61 | 14.92 | 11.64 |
| 258 | GLY | N | −3.47 | 73.02 | 16.39 | 11.23 |
| 258 | GLY | CA | −2.37 | 73.32 | 17.29 | 11.01 |
| 258 | GLY | C | −1.06 | 72.77 | 16.75 | 10.72 |
| 258 | GLY | O | −1.04 | 72.01 | 15.79 | 10.58 |
| 259 | LEU | N | 0.04 | 73.16 | 17.39 | 10.52 |
| 259 | LEU | CA | 1.35 | 72.71 | 16.97 | 10.29 |
| 259 | LEU | CB | 2.43 | 73.57 | 17.62 | 10.68 |
| 259 | LEU | CG | 2.43 | 75.06 | 17.28 | 11.00 |
| 259 | LEU | CD1 | 3.45 | 75.77 | 18.13 | 11.39 |
| 259 | LEU | CD2 | 2.72 | 75.25 | 15.80 | 11.28 |
| 259 | LEU | C | 1.61 | 71.25 | 17.33 | 9.92 |
| 259 | LEU | O | 0.99 | 70.70 | 18.24 | 9.95 |
| 260 | SER | N | 2.54 | 70.65 | 16.60 | 9.52 |
| 260 | SER | CA | 2.96 | 69.27 | 16.83 | 9.19 |
| 260 | SER | CB | 2.35 | 68.33 | 15.80 | 9.10 |
| 260 | SER | OG | 2.69 | 66.98 | 16.08 | 8.89 |
| 260 | SER | C | 4.48 | 69.23 | 16.71 | 8.96 |
| 260 | SER | O | 5.07 | 70.01 | 15.97 | 8.89 |
| 261 | CYS | N | 5.11 | 68.32 | 17.45 | 8.81 |
| 261 | CYS | CA | 6.55 | 68.18 | 17.38 | 8.69 |
| 261 | CYS | CB | 7.24 | 68.96 | 18.51 | 8.87 |
| 261 | CYS | SG | 9.02 | 69.20 | 18.27 | 9.13 |
| 261 | CYS | C | 6.92 | 66.70 | 17.48 | 8.59 |
| 261 | CYS | O | 6.18 | 65.91 | 18.08 | 8.57 |
| 262 | GLN | N | 8.05 | 66.34 | 16.90 | 8.55 |
| 262 | GLN | CA | 8.53 | 64.97 | 16.91 | 8.61 |
| 262 | GLN | CB | 8.03 | 64.22 | 15.66 | 8.59 |
| 262 | GLN | CG | 8.54 | 62.79 | 15.56 | 8.64 |
| 262 | GLN | CD | 8.01 | 62.03 | 14.37 | 8.74 |
| 262 | GLN | OE1 | 7.89 | 62.58 | 13.27 | 8.83 |
| 262 | GLN | NE2 | 7.71 | 60.76 | 14.57 | 8.97 |
| 262 | GLN | C | 10.05 | 64.91 | 16.94 | 8.68 |
| 262 | GLN | O | 10.72 | 65.70 | 16.28 | 8.76 |
| 263 | TYR | N | 10.56 | 63.97 | 17.73 | 8.79 |
| 263 | TYR | CA | 11.99 | 63.69 | 17.82 | 8.94 |
| 263 | TYR | CB | 12.62 | 64.16 | 19.13 | 9.14 |
| 263 | TYR | CG | 12.92 | 65.63 | 19.22 | 9.24 |
| 263 | TYR | CD1 | 11.96 | 66.53 | 19.68 | 9.36 |
| 263 | TYR | CE1 | 12.25 | 67.88 | 19.83 | 9.56 |
| 263 | TYR | CD2 | 14.18 | 66.12 | 18.89 | 9.43 |
| 263 | TYR | CE2 | 14.48 | 67.48 | 19.02 | 9.54 |
| 263 | TYR | CZ | 13.51 | 68.35 | 19.50 | 9.60 |
| 263 | TYR | OH | 13.80 | 69.68 | 19.69 | 9.93 |
| 263 | TYR | C | 12.07 | 62.18 | 17.74 | 8.97 |
| 263 | TYR | O | 11.19 | 61.48 | 18.25 | 8.99 |
| 264 | GLU | N | 13.13 | 61.67 | 17.12 | 9.04 |
| 264 | GLU | CA | 13.29 | 60.24 | 16.95 | 9.15 |
| 264 | GLU | CB | 12.64 | 59.82 | 15.63 | 9.25 |
| 264 | GLU | CG | 13.00 | 58.43 | 15.13 | 9.25 |
| 264 | GLU | CD | 12.35 | 58.11 | 13.80 | 9.20 |
| 264 | GLU | OE1 | 12.21 | 59.04 | 12.97 | 9.12 |
| 264 | GLU | OE2 | 11.98 | 56.93 | 13.59 | 9.27 |
| 264 | GLU | C | 14.73 | 59.77 | 16.95 | 9.16 |
| 264 | GLU | O | 15.63 | 60.46 | 16.45 | 9.36 |
| 265 | HIS | N | 14.95 | 58.59 | 17.51 | 9.11 |
| 265 | HIS | CA | 16.25 | 57.95 | 17.50 | 9.08 |
| 265 | HIS | CB | 16.99 | 58.07 | 18.83 | 9.19 |
| 265 | HIS | CG | 17.87 | 59.27 | 18.94 | 9.18 |
| 265 | HIS | CD2 | 18.01 | 60.18 | 19.93 | 9.33 |
| 265 | HIS | ND1 | 18.77 | 59.64 | 17.96 | 9.29 |
| 265 | HIS | CE1 | 19.41 | 60.72 | 18.34 | 9.31 |
| 265 | HIS | NE2 | 18.97 | 61.07 | 19.54 | 9.39 |
| 265 | HIS | C | 16.04 | 56.48 | 17.22 | 9.09 |
| 265 | HIS | O | 15.03 | 55.90 | 17.63 | 8.96 |
| 266 | GLN | N | 16.99 | 55.90 | 16.50 | 9.21 |
| 266 | GLN | CA | 16.99 | 54.48 | 16.20 | 9.46 |
| 266 | GLN | CB | 17.42 | 54.24 | 14.76 | 9.57 |
| 266 | GLN | CG | 17.52 | 52.77 | 14.37 | 9.74 |
| 266 | GLN | CD | 17.82 | 52.59 | 12.89 | 9.79 |
| 266 | GLN | OE1 | 18.63 | 51.74 | 12.50 | 10.19 |
| 266 | GLN | NE2 | 17.16 | 53.39 | 12.06 | 9.62 |
| 266 | GLN | C | 18.05 | 53.94 | 17.17 | 9.57 |
| 266 | GLN | O | 19.09 | 54.57 | 17.35 | 9.60 |
| 267 | PHE | N | 17.77 | 52.81 | 17.79 | 9.85 |
| 267 | PHE | CA | 18.71 | 52.25 | 18.75 | 10.21 |
| 267 | PHE | CB | 18.40 | 52.80 | 20.15 | 10.24 |
| 267 | PHE | CG | 17.00 | 52.49 | 20.63 | 10.27 |
| 267 | PHE | CD1 | 16.71 | 51.30 | 21.28 | 10.40 |
| 267 | PHE | CD2 | 15.96 | 53.38 | 20.37 | 10.31 |
| 267 | PHE | CE1 | 15.41 | 50.99 | 21.67 | 10.49 |
| 267 | PHE | CE2 | 14.66 | 53.09 | 20.76 | 10.40 |
| 267 | PHE | CZ | 14.38 | 51.89 | 21.41 | 10.47 |
| 267 | PHE | C | 18.62 | 50.73 | 18.78 | 10.47 |
| 267 | PHE | O | 17.66 | 50.15 | 18.29 | 10.36 |
| 268 | VAL | N | 19.64 | 50.11 | 19.35 | 10.90 |
| 268 | VAL | CA | 19.65 | 48.66 | 19.47 | 11.46 |
| 268 | VAL | CB | 20.91 | 48.04 | 18.81 | 11.56 |
| 268 | VAL | CG1 | 22.18 | 48.62 | 19.42 | 11.74 |
| 268 | VAL | CG2 | 20.88 | 46.52 | 18.98 | 11.70 |
| 268 | VAL | C | 19.63 | 48.34 | 20.96 | 11.85 |
| 268 | VAL | O | 20.28 | 49.01 | 21.77 | 11.76 |
| 269 | ILE | N | 18.86 | 47.33 | 21.33 | 12.39 |
| 269 | ILE | CA | 18.79 | 46.93 | 22.72 | 13.05 |
| 269 | ILE | CB | 17.43 | 46.27 | 23.03 | 13.14 |
| 269 | ILE | CG2 | 17.43 | 45.72 | 24.45 | 13.25 |
| 269 | ILE | CG1 | 16.32 | 47.30 | 22.83 | 13.27 |
| 269 | ILE | CD1 | 14.93 | 46.78 | 23.10 | 13.40 |
| 269 | ILE | C | 19.94 | 45.96 | 22.99 | 13.45 |
| 269 | ILE | O | 20.05 | 44.92 | 22.35 | 13.46 |
| 270 | THR | N | 20.81 | 46.33 | 23.93 | 14.04 |
| 270 | THR | CA | 21.95 | 45.49 | 24.29 | 14.61 |
| 270 | THR | CB | 23.29 | 46.23 | 24.12 | 14.61 |
| 270 | THR | OG1 | 23.49 | 47.12 | 25.23 | 14.59 |
| 270 | THR | CG2 | 23.32 | 47.02 | 22.82 | 14.69 |
| 270 | THR | C | 21.80 | 45.06 | 25.74 | 15.00 |
| 270 | THR | O | 20.98 | 45.61 | 26.47 | 15.04 |
| 271 | LYS | N | 22.62 | 44.10 | 26.16 | 15.49 |
| 271 | LYS | CA | 22.56 | 43.62 | 27.54 | 15.96 |
| 271 | LYS | CB | 23.54 | 42.46 | 27.74 | 16.30 |
| 271 | LYS | CG | 25.03 | 42.83 | 27.81 | 16.76 |
| 271 | LYS | CD | 25.63 | 43.16 | 26.45 | 17.18 |
| 271 | LYS | CE | 25.45 | 44.62 | 26.09 | 17.40 |
| 271 | LYS | NZ | 26.07 | 44.95 | 24.77 | 17.64 |
| 271 | LYS | C | 22.85 | 44.73 | 28.54 | 16.05 |
| 271 | LYS | O | 22.38 | 44.67 | 29.68 | 16.26 |
| 272 | ASP | N | 23.60 | 45.73 | 28.12 | 16.07 |
| 272 | ASP | CA | 23.95 | 46.84 | 29.00 | 16.03 |
| 272 | ASP | CB | 25.42 | 47.22 | 28.80 | 16.49 |
| 272 | ASP | CG | 26.36 | 46.11 | 29.23 | 16.83 |
| 272 | ASP | OD1 | 26.33 | 45.75 | 30.43 | 17.17 |
| 272 | ASP | OD2 | 27.12 | 45.60 | 28.39 | 17.21 |
| 272 | ASP | C | 23.08 | 48.08 | 28.82 | 15.68 |
| 272 | ASP | O | 33.34 | 49.12 | 29.41 | 15.83 |
| 273 | GLY | N | 22.03 | 47.95 | 28.01 | 15.16 |
| 273 | GLY | CA | 21.14 | 49.08 | 27.80 | 14.48 |
| 273 | GLY | C | 21.00 | 49.48 | 26.34 | 13.97 |
| 273 | GLY | O | 21.74 | 48.99 | 25.49 | 13.95 |
| 274 | PRO | N | 20.06 | 50.37 | 26.03 | 13.54 |
| 274 | PRO | CD | 19.08 | 51.02 | 26.92 | 13.49 |
| 274 | PRO | CA | 19.88 | 50.80 | 24.64 | 13.12 |
| 274 | PRO | CB | 18.56 | 51.58 | 24.70 | 13.24 |
| 274 | PRO | CG | 18.59 | 52.17 | 26.07 | 13.39 |
| 274 | PRO | C | 21.04 | 51.65 | 24.14 | 12.74 |
| 274 | PRO | O | 21.54 | 52.53 | 24.84 | 12.77 |
| 275 | VAL | N | 21.48 | 51.37 | 22.92 | 12.25 |
| 275 | VAL | CA | 22.57 | 52.12 | 22.31 | 11.83 |
| 275 | VAL | CB | 23.74 | 51.20 | 21.89 | 11.99 |
| 275 | VAL | CG1 | 24.82 | 52.01 | 21.19 | 12.20 |
| 275 | VAL | CG2 | 24.32 | 50.51 | 23.12 | 12.24 |
| 275 | VAL | C | 22.03 | 52.85 | 21.09 | 11.34 |
| 275 | VAL | O | 21.59 | 52.22 | 20.12 | 11.15 |
| 276 | ILE | N | 22.04 | 54.17 | 21.15 | 10.91 |
| 276 | ILE | CA | 21.52 | 55.00 | 20.07 | 10.57 |
| 276 | ILE | CB | 21.31 | 56.45 | 20.57 | 10.56 |
| 276 | ILE | CG2 | 20.93 | 57.36 | 19.41 | 10.51 |
| 276 | ILE | CG1 | 20.21 | 56.45 | 21.63 | 10.65 |
| 276 | ILE | CD1 | 20.09 | 57.75 | 22.40 | 10.79 |
| 276 | ILE | C | 22.45 | 54.96 | 18.86 | 10.36 |
| 276 | ILE | O | 23.65 | 55.22 | 18.96 | 10.46 |
| 277 | LEU | N | 21.86 | 54.65 | 17.70 | 10.11 |
| 277 | LEU | CA | 22.58 | 54.52 | 16.44 | 9.96 |
| 277 | LEU | CB | 21.98 | 53.35 | 15.65 | 10.19 |
| 277 | LEU | CG | 22.04 | 52.00 | 16.36 | 10.38 |
| 277 | LEU | CD1 | 21.06 | 51.02 | 15.74 | 10.55 |
| 277 | LEU | CD2 | 23.46 | 51.47 | 16.30 | 10.71 |
| 277 | LEU | C | 22.57 | 55.77 | 15.58 | 9.76 |
| 277 | LEU | O | 23.31 | 55.87 | 14.60 | 9.72 |
| 278 | THR | N | 21.73 | 56.74 | 15.95 | 9.57 |
| 278 | THR | CA | 21.59 | 57.99 | 15.22 | 9.54 |
| 278 | THR | CB | 20.13 | 58.17 | 14.78 | 9.44 |
| 278 | THR | OG1 | 19.26 | 57.89 | 15.88 | 9.20 |
| 278 | THR | CG2 | 19.80 | 57.21 | 13.63 | 9.37 |
| 278 | THR | C | 21.99 | 59.19 | 16.09 | 9.65 |
| 278 | THR | O | 21.44 | 60.28 | 15.95 | 9.56 |
| 279 | SER | N | 22.96 | 58.98 | 16.98 | 9.91 |
| 279 | SER | CA | 23.39 | 60.04 | 17.88 | 10.19 |
| 279 | SER | CB | 24.26 | 59.45 | 19.00 | 10.29 |
| 279 | SER | OG | 24.76 | 60.48 | 19.84 | 10.44 |
| 279 | SER | C | 24.16 | 61.18 | 17.22 | 10.39 |
| 279 | SER | O | 25.05 | 60.95 | 16.41 | 10.38 |
| 280 | GLN | N | 23.80 | 62.40 | 17.60 | 10.64 |
| 280 | GLN | CA | 24.47 | 63.59 | 17.07 | 11.05 |
| 280 | GLN | CB | 23.44 | 64.69 | 16.79 | 10.71 |
| 280 | GLN | CG | 22.45 | 64.37 | 15.69 | 10.38 |
| 280 | GLN | CD | 21.30 | 65.34 | 15.64 | 10.14 |
| 280 | GLN | OE1 | 21.48 | 66.56 | 15.75 | 10.09 |
| 280 | GLN | NE2 | 20.09 | 64.82 | 15.46 | 9.95 |
| 280 | GLN | C | 25.50 | 64.12 | 18.07 | 11.50 |
| 280 | GLN | O | 26.16 | 65.12 | 17.82 | 11.62 |
| 281 | GLY | N | 25.63 | 63.44 | 19.21 | 12.05 |
| 281 | GLY | CA | 26.57 | 63.85 | 20.23 | 12.81 |
| 281 | GLY | C | 26.07 | 63.49 | 21.62 | 13.31 |
| 281 | GLY | O | 25.24 | 62.58 | 21.76 | 13.32 |
| 282 | GLU | N | 26.56 | 64.17 | 22.65 | 13.84 |
| 282 | GLU | CA | 26.14 | 63.90 | 24.02 | 14.38 |
| 282 | GLU | CB | 26.78 | 64.90 | 24.99 | 14.80 |
| 282 | GLU | CG | 26.66 | 64.51 | 26.45 | 15.53 |
| 282 | GLU | CD | 27.20 | 65.58 | 27.38 | 15.90 |
| 282 | GLU | OE1 | 28.22 | 66.22 | 27.03 | 16.30 |
| 282 | GLU | OE2 | 26.62 | 65.77 | 28.47 | 16.31 |
| 282 | GLU | C | 24.62 | 64.03 | 24.07 | 14.45 |
| 282 | GLU | O | 24.08 | 65.09 | 23.77 | 14.46 |
| 283 | GLU | N | 23.94 | 62.96 | 24.46 | 14.56 |
| 283 | GLU | CA | 22.48 | 62.98 | 24.50 | 14.61 |
| 283 | GLU | CB | 21.93 | 61.61 | 24.92 | 14.32 |
| 283 | GLU | CG | 22.40 | 60.44 | 24.07 | 13.88 |
| 283 | GLU | CD | 22.30 | 60.68 | 22.57 | 13.57 |
| 283 | GLU | OE1 | 21.47 | 61.51 | 22.14 | 13.27 |
| 283 | GLU | OE2 | 23.05 | 60.03 | 21.82 | 13.29 |
| 283 | GLU | C | 21.91 | 64.06 | 25.42 | 14.85 |
| 283 | GLU | O | 22.37 | 64.25 | 26.54 | 14.99 |
| 284 | GLY | N | 20.88 | 64.74 | 24.91 | 15.03 |
| 284 | GLY | CA | 20.22 | 65.78 | 25.67 | 15.24 |
| 284 | GLY | C | 20.78 | 67.17 | 25.43 | 15.30 |
| 284 | GLY | O | 20.16 | 68.16 | 25.82 | 15.48 |
| 285 | THR | N | 21.93 | 67.26 | 24.78 | 15.29 |
| 285 | THR | CA | 22.57 | 68.55 | 24.51 | 15.19 |
| 285 | THR | CB | 24.10 | 68.45 | 24.66 | 15.41 |
| 285 | THR | OG1 | 24.63 | 67.63 | 23.62 | 15.63 |
| 285 | THR | CG2 | 24.46 | 67.85 | 26.02 | 15.53 |
| 285 | THR | C | 22.28 | 69.15 | 23.14 | 14.89 |
| 285 | THR | O | 22.80 | 70.22 | 22.81 | 15.02 |
| 286 | TYR | N | 21.46 | 68.47 | 22.34 | 14.43 |
| 286 | TYR | CA | 21.14 | 68.97 | 21.00 | 13.94 |
| 286 | TYR | CB | 22.06 | 68.31 | 19.97 | 13.50 |
| 286 | TYR | CG | 21.93 | 66.80 | 19.91 | 12.93 |
| 286 | TYR | CD1 | 20.99 | 66.20 | 19.08 | 12.69 |
| 286 | TYR | CE1 | 20.86 | 64.81 | 19.02 | 12.33 |
| 286 | TYR | CD2 | 22.75 | 65.97 | 20.68 | 12.75 |
| 286 | TYR | CE2 | 22.63 | 64.59 | 20.63 | 12.40 |
| 286 | TYR | CZ | 21.68 | 64.02 | 19.79 | 12.25 |
| 286 | TYR | OH | 21.56 | 62.65 | 19.71 | 11.82 |
| 286 | TYR | C | 19.68 | 68.73 | 20.66 | 13.86 |
| 286 | TYR | O | 18.99 | 68.07 | 21.46 | 13.85 |
| 286 | TYR | OT1 | 19.23 | 69.21 | 19.59 | 13.77 |
| 314 | HOH | O | 25.62 | 55.76 | 14.77 | 6.45 |
| 315 | HOH | O | 19.71 | 62.07 | 14.66 | 10.04 |
| 316 | HOH | O | 7.77 | 55.56 | 19.39 | 10.43 |
| 317 | HOH | O | 3.09 | 68.32 | 2.12 | 11.83 |
| 318 | HOH | O | 8.58 | 65.39 | 9.46 | 10.26 |
| 319 | HOH | O | 1.83 | 56.58 | 19.07 | 10.84 |
| 321 | HOH | O | 25.04 | 56.74 | 17.00 | 10.83 |
| 322 | HOH | O | 5.29 | 66.33 | 15.09 | 10.00 |
| 323 | HOH | O | −4.26 | 71.72 | 11.39 | 13.32 |
| 324 | HOH | O | 2.94 | 60.11 | 19.41 | 11.38 |
| 325 | HOH | O | 4.26 | 51.36 | 23.80 | 11.70 |
| 326 | HOH | O | 11.05 | 74.47 | −8.06 | 12.27 |
| 327 | HOH | O | 7.63 | 65.04 | 11.99 | 8.99 |
| 328 | HOH | O | 8.02 | 61.07 | 11.02 | 9.06 |
| 329 | HOH | O | 3.55 | 45.00 | 22.11 | 12.90 |
| 330 | HOH | O | 15.14 | 59.07 | 2.21 | 9.87 |
| 332 | HOH | O | 0.50 | 57.96 | 1.35 | 10.07 |
| 333 | HOH | O | 31.03 | 44.64 | 9.37 | 13.64 |
| 334 | HOH | O | 5.86 | 77.62 | 15.40 | 12.45 |
| 335 | HOH | O | 29.15 | 56.87 | 8.75 | 12.35 |
| 336 | HOH | O | −0.47 | 58.78 | −1.28 | 11.81 |
| 337 | HOH | O | 7.03 | 69.60 | −2.88 | 15.16 |
| 338 | HOH | O | −3.31 | 60.11 | −7.59 | 13.69 |
| 339 | HOH | O | 0.20 | 58.17 | −8.65 | 10.69 |
| 340 | HOH | O | 30.78 | 56.80 | 1.88 | 13.04 |
| 341 | HOH | O | 0.23 | 54.29 | −5.91 | 10.84 |
| 342 | HOH | O | 0.00 | 54.02 | 31.24 | 15.18 |
| 343 | HOH | O | 27.34 | 48.05 | −1.33 | 11.07 |
| 344 | HOH | O | 17.05 | 71.62 | 17.19 | 11.59 |
| 345 | HOH | O | 30.92 | 54.46 | −1.29 | 13.42 |
| 346 | HOH | O | 16.45 | 74.31 | 17.01 | 13.33 |
| 347 | HOH | O | −3.50 | 60.43 | 16.50 | 12.93 |
| 348 | HOH | O | 2.60 | 52.75 | −5.83 | 12.71 |
| 349 | HOH | O | 24.41 | 57.86 | 22.69 | 16.07 |
| 350 | HOH | O | 20.93 | 68.90 | −4.11 | 12.68 |
| 351 | HOH | O | 4.43 | 57.87 | −7.65 | 12.98 |
| 352 | HOH | O | −3.55 | 68.44 | 25.36 | 16.96 |
| 353 | HOH | O | −3.51 | 46.12 | 27.66 | 15.41 |
| 354 | HOH | O | 22.20 | 46.46 | 8.94 | 13.79 |
| 355 | HOH | O | 22.93 | 71.60 | −1.05 | 15.48 |
| 356 | HOH | O | −3.01 | 62.19 | 24.73 | 13.30 |
| 357 | HOH | O | 22.88 | 45.00 | 6.72 | 13.28 |
| 358 | HOH | O | 6.45 | 54.38 | −8.47 | 14.25 |
| 359 | HOH | O | 26.36 | 50.78 | 6.62 | 14.16 |
| 360 | HOH | O | 14.66 | 70.38 | 22.56 | 17.51 |
| 361 | HOH | O | 5.96 | 56.76 | −9.70 | 14.63 |
| 362 | HOH | O | 7.80 | 62.74 | 8.86 | 10.67 |
| 363 | HOH | O | 7.09 | 53.05 | 32.05 | 13.64 |
| 364 | HOH | O | 25.66 | 67.75 | 18.62 | 14.72 |
| 365 | HOH | O | 19.61 | 65.67 | 22.69 | 14.17 |
| 366 | HOH | O | 5.61 | 60.10 | −6.56 | 13.66 |
| 367 | HOH | O | 19.50 | 44.33 | 9.43 | 12.20 |
| 368 | HOH | O | 2.04 | 49.75 | 23.74 | 14.45 |
| 369 | HOH | O | 30.02 | 57.15 | 4.45 | 16.93 |
| 370 | HOH | O | 4.07 | 74.27 | 24.72 | 12.60 |
| 371 | HOH | O | 1.60 | 69.84 | 3.83 | 16.22 |
| 372 | HOH | O | 27.75 | 58.99 | −5.61 | 14.15 |
| 373 | HOH | O | 20.41 | 73.61 | −4.06 | 18.67 |
| 374 | HOH | O | 29.68 | 48.03 | 8.00 | 13.57 |
| 375 | HOH | O | 11.84 | 62.90 | −6.57 | 14.25 |
| 376 | HOH | O | −5.98 | 60.61 | 15.01 | 16.51 |
| 377 | HOH | O | 6.53 | 51.96 | −9.71 | 13.76 |
| 378 | HOH | O | 29.59 | 55.43 | 6.52 | 14.50 |
| 379 | HOH | O | −3.29 | 59.92 | 23.22 | 12.68 |
| 380 | HOH | O | 7.44 | 80.80 | 17.52 | 14.85 |
| 381 | HOH | O | −0.32 | 50.95 | 23.16 | 16.14 |
| 382 | HOH | O | 9.02 | 50.85 | −10.01 | 14.59 |
| 383 | HOH | O | 18.27 | 65.67 | −7.56 | 17.17 |
| 384 | HOH | O | 2.49 | 76.71 | 9.26 | 17.89 |
| 385 | HOH | O | −0.27 | 72.64 | 25.56 | 17.47 |
| 386 | HOH | O | 19.13 | 46.16 | 28.38 | 16.29 |
| 387 | HOH | O | 15.73 | 65.96 | 26.72 | 15.71 |
| 388 | HOH | O | 16.49 | 70.41 | 19.60 | 14.12 |
| 389 | HOH | O | 3.09 | 69.12 | 34.79 | 17.17 |
| 390 | HOH | O | 10.94 | 85.73 | 9.20 | 17.07 |
| 391 | HOH | O | 12.67 | 44.52 | 13.98 | 16.08 |
| 392 | HOH | O | 31.71 | 53.61 | 6.43 | 13.68 |
| 393 | HOH | O | 1.47 | 56.29 | 31.56 | 17.83 |
| 394 | HOH | O | 13.03 | 42.61 | 11.83 | 18.02 |
| 395 | HOH | O | 0.91 | 49.01 | 26.18 | 15.79 |
| 396 | HOH | O | 14.28 | 68.25 | 26.50 | 13.86 |
| 397 | HOH | O | 1.07 | 52.18 | 33.02 | 15.45 |
| 398 | HOH | O | 11.78 | 56.39 | 31.65 | 14.05 |
| 399 | HOH | O | 9.24 | 39.28 | 22.61 | 14.96 |
| 400 | HOH | O | 4.52 | 52.40 | 2.46 | 13.42 |
| 401 | HOH | O | 24.78 | 40.94 | 7.52 | 15.67 |
| 402 | HOH | O | 9.80 | 60.21 | 7.67 | 17.48 |
| 403 | HOH | O | 19.53 | 70.60 | 16.80 | 19.96 |
| 404 | HOH | O | 4.51 | 48.49 | −4.55 | 13.61 |
| 405 | HOH | O | 8.88 | 60.40 | −11.11 | 18.67 |
| 406 | HOH | O | 18.12 | 52.54 | 30.33 | 19.37 |
| 407 | HOH | O | −1.43 | 47.64 | 26.58 | 20.92 |
| 408 | HOH | O | 7.35 | 50.68 | 33.44 | 17.13 |
| 409 | HOH | O | 28.20 | 66.59 | 22.26 | 22.52 |
| 410 | HOH | O | 24.98 | 39.30 | 9.73 | 17.60 |
| 411 | HOH | O | 17.19 | 79.62 | 0.27 | 17.92 |
| 412 | HOH | O | 28.11 | 39.55 | 13.03 | 19.37 |
| 413 | HOH | O | 3.97 | 66.56 | 35.23 | 15.09 |
| 414 | HOH | O | 14.78 | 44.15 | 15.58 | 15.13 |
| 415 | HOH | O | 17.33 | 57.88 | 27.34 | 17.14 |
| 416 | HOH | O | 12.90 | 79.65 | 24.70 | 16.70 |
| 417 | HOH | O | −2.63 | 67.22 | 6.11 | 20.55 |
| 418 | HOH | O | 27.79 | 69.95 | 5.47 | 21.96 |
| 419 | HOH | O | 19.14 | 63.10 | −9.29 | 13.90 |
| 420 | HOH | O | 23.68 | 45.09 | −0.56 | 21.61 |
| 421 | HOH | O | 22.14 | 71.39 | −3.61 | 15.32 |
| 422 | HOH | O | −4.23 | 58.88 | 10.66 | 23.46 |
| 423 | HOH | O | 1.90 | 47.27 | 22.61 | 21.15 |
| 424 | HOH | O | 17.49 | 39.11 | 25.29 | 17.82 |
| 425 | HOH | O | −5.48 | 67.75 | 17.10 | 15.68 |
| 426 | HOH | O | 7.39 | 50.64 | −0.53 | 15.87 |
| 427 | HOH | O | 11.13 | 54.18 | 8.65 | 17.67 |
| 428 | HOH | O | 23.17 | 55.55 | 23.46 | 15.47 |
| 429 | HOH | O | −2.21 | 58.01 | 3.82 | 18.25 |
| 430 | HOH | O | 11.33 | 73.53 | 28.12 | 15.62 |
| 431 | HOH | O | 0.62 | 72.27 | 28.21 | 21.53 |
| 432 | HOH | O | 7.70 | 45.27 | 32.27 | 19.69 |
| 433 | HOH | O | 6.87 | 73.81 | −3.87 | 17.81 |
| 434 | HOH | O | 4.17 | 62.70 | 36.21 | 18.21 |
| 435 | HOH | O | 11.27 | 79.11 | 16.02 | 15.79 |
| 436 | HOH | O | −6.39 | 69.33 | 10.15 | 22.08 |
| 437 | HOH | O | 31.26 | 49.21 | −12.14 | 20.40 |
| 438 | HOH | O | 4.27 | 46.02 | 19.65 | 15.49 |
| 439 | HOH | O | 31.55 | 41.96 | 9.49 | 19.34 |
| 440 | HOH | O | 11.22 | 84.88 | 6.53 | 18.07 |
| 441 | HOH | O | 22.02 | 40.78 | 7.03 | 18.67 |
| 442 | HOH | O | 3.08 | 72.05 | 31.31 | 21.08 |
| 443 | HOH | O | 25.49 | 56.12 | 20.89 | 20.37 |
| 444 | HOH | O | 7.18 | 81.53 | 1.11 | 18.85 |
| 445 | HOH | O | 1.47 | 74.61 | 24.45 | 19.91 |
| 446 | HOH | O | 0.70 | 73.42 | 5.81 | 22.03 |
| 447 | HOH | O | 26.78 | 38.90 | 16.38 | 22.73 |
| 448 | HOH | O | 3.61 | 45.02 | 17.14 | 18.57 |
| 449 | HOH | O | 9.55 | 57.27 | 32.97 | 15.50 |
| 450 | HOH | O | 33.45 | 53.77 | 8.57 | 14.93 |
| 451 | HOH | O | 9.70 | 38.74 | 19.96 | 20.16 |
| 452 | HOH | O | 4.42 | 70.35 | −2.38 | 20.63 |
| 453 | HOH | O | 14.95 | 75.11 | 25.96 | 17.69 |
| 454 | HOH | O | 25.38 | 53.64 | −11.16 | 22.80 |
| 455 | HOH | O | 28.54 | 45.80 | 16.18 | 20.64 |
| 456 | HOH | O | 2.03 | 65.80 | 1.47 | 20.62 |
| 457 | HOH | O | 18.75 | 75.70 | 12.69 | 22.34 |
| 458 | HOH | O | 17.48 | 79.35 | 12.91 | 19.93 |
| 459 | HOH | O | 20.64 | 43.53 | 6.59 | 19.01 |
| 460 | HOH | O | −2.49 | 58.34 | 25.35 | 19.38 |
| 461 | HOH | O | −7.22 | 72.24 | 21.40 | 19.81 |
| 462 | HOH | O | 14.62 | 85.03 | 1.42 | 23.03 |
| 463 | HOH | O | −2.15 | 50.13 | 25.20 | 20.07 |
| 464 | HOH | O | 5.24 | 53.40 | −12.01 | 20.22 |
| 465 | HOH | O | 24.19 | 62.36 | −13.11 | 15.83 |
| 466 | HOH | O | 21.09 | 46.56 | −1.93 | 22.13 |
| 467 | HOH | O | −0.31 | 75.21 | 19.44 | 17.33 |
| 468 | HOH | O | 29.36 | 59.62 | 8.81 | 20.27 |
| 469 | HOH | O | 27.90 | 67.78 | 9.73 | 19.54 |
| 470 | HOH | O | 13.31 | 67.69 | −7.50 | 22.51 |
| 471 | HOH | O | −2.52 | 54.24 | 27.56 | 18.21 |
| 472 | HOH | O | 19.59 | 54.60 | 29.40 | 21.25 |
| 473 | HOH | O | −5.70 | 60.97 | 1.77 | 24.93 |
| 474 | HOH | O | 1.52 | 42.24 | 32.47 | 20.62 |
| 475 | HOH | O | 31.78 | 56.83 | −2.20 | 19.80 |
| 476 | HOH | O | 7.47 | 83.72 | 23.89 | 21.43 |
| 477 | HOH | O | 6.28 | 63.24 | 34.52 | 16.16 |
| 478 | HOH | O | 3.04 | 64.99 | 37.33 | 20.01 |
| 479 | HOH | O | 5.99 | 59.55 | 12.06 | 19.34 |
| 480 | HOH | O | 20.93 | 72.99 | 0.15 | 18.98 |
| 481 | HOH | O | 7.72 | 55.28 | 33.48 | 22.07 |
| 482 | HOH | O | 16.95 | 74.17 | −5.66 | 20.90 |
| 483 | HOH | O | 33.43 | 49.96 | −4.87 | 21.06 |
| 484 | HOH | O | 5.67 | 62.29 | −8.07 | 20.76 |
| 485 | HOH | O | −3.50 | 56.43 | 2.19 | 17.54 |
| 486 | HOH | O | 18.75 | 47.78 | 25.97 | 19.25 |
| 487 | HOH | O | 25.48 | 60.77 | 25.80 | 21.32 |
| 488 | HOH | O | 7.80 | 58.03 | −11.36 | 20.49 |
| 489 | HOH | O | 7.54 | 83.13 | 4.21 | 18.81 |
| 490 | HOH | O | 22.16 | 66.88 | −5.27 | 23.37 |
| 491 | HOH | O | 9.97 | 50.45 | 34.16 | 20.41 |
| 492 | HOH | O | 6.26 | 50.35 | 1.93 | 21.49 |
| 493 | HOH | O | −2.25 | 70.37 | 18.92 | 16.53 |
| 494 | HOH | O | 17.82 | 41.67 | 9.84 | 22.92 |
| 495 | HOH | O | 16.95 | 78.58 | 16.18 | 22.58 |
| 496 | HOH | O | −0.45 | 67.19 | 35.90 | 21.51 |
| 497 | HOH | O | 26.13 | 68.33 | 21.37 | 20.98 |
| 498 | HOH | O | −0.73 | 50.81 | 34.63 | 24.81 |
| 499 | HOH | O | 30.75 | 56.45 | 11.00 | 21.52 |
| 500 | HOH | O | 8.59 | 42.77 | 10.47 | 16.92 |
| 501 | HOH | O | 7.62 | 60.95 | 34.08 | 20.29 |
| 502 | HOH | O | −0.72 | 69.16 | −0.51 | 21.80 |
| 503 | HOH | O | −4.11 | 56.13 | 25.96 | 20.12 |
| 504 | HOH | O | 3.83 | 71.47 | 34.02 | 22.03 |
| 505 | HOH | O | 25.36 | 73.24 | 6.50 | 24.79 |
| 506 | HOH | O | 31.78 | 46.62 | −5.92 | 21.25 |
| 507 | HOH | O | 0.03 | 42.48 | 27.49 | 22.25 |
| 508 | HOH | O | 3.44 | 70.15 | −0.02 | 22.38 |
| 509 | HOH | O | 4.60 | 81.12 | 17.60 | 22.00 |
| 510 | HOH | O | 4.26 | 45.69 | −4.29 | 21.06 |
| 511 | HOH | O | 14.73 | 50.32 | 2.07 | 21.60 |
| 512 | HOH | O | 20.15 | 50.62 | 30.68 | 21.15 |
| 513 | HOH | O | 12.05 | 67.07 | 32.27 | 22.10 |
| 514 | HOH | O | −0.64 | 75.97 | 15.09 | 23.07 |
| 515 | HOH | O | 20.00 | 47.71 | 30.91 | 21.88 |
| 516 | HOH | O | 26.56 | 72.54 | 12.35 | 22.54 |
| 517 | HOH | O | 13.52 | 69.16 | 29.05 | 19.15 |
| 518 | HOH | O | 27.45 | 60.31 | 20.30 | 21.45 |
| 519 | HOH | O | 8.00 | 59.40 | 32.00 | 19.52 |
| 520 | HOH | O | 15.39 | 85.53 | 15.74 | 23.07 |
| 521 | HOH | O | 5.83 | 56.55 | 12.59 | 24.73 |
| 522 | HOH | O | 19.04 | 44.91 | 4.74 | 21.60 |
| 523 | HOH | O | 28.06 | 61.53 | 13.01 | 22.18 |
| 524 | HOH | O | 19.98 | 39.23 | 26.66 | 22.96 |
| 525 | HOH | O | 6.73 | 83.88 | 11.98 | 19.98 |
| 526 | HOH | O | −0.09 | 58.45 | 32.08 | 20.31 |
| 527 | HOH | O | 3.61 | 78.95 | 16.26 | 19.95 |
| 528 | HOH | O | −4.79 | 64.05 | 25.65 | 22.66 |
| 529 | HOH | O | 23.72 | 64.14 | −9.62 | 20.87 |
| 530 | HOH | O | −2.56 | 74.11 | 8.29 | 22.03 |
| 531 | HOH | O | 30.90 | 44.14 | 13.46 | 23.73 |
| 532 | HOH | O | 5.51 | 65.89 | −5.22 | 21.87 |
| 533 | HOH | O | 6.81 | 47.79 | −7.93 | 22.41 |
| 534 | HOH | O | 23.37 | 56.01 | −13.72 | 23.15 |
| 535 | HOH | O | −10.60 | 71.39 | 16.90 | 21.76 |
| 536 | HOH | O | 17.32 | 72.28 | 21.42 | 23.04 |
| 537 | HOH | O | 15.02 | 51.36 | 32.82 | 23.81 |
| 538 | HOH | O | 5.40 | 53.27 | 12.14 | 21.09 |
| 540 | HOH | O | 18.96 | 55.80 | 27.01 | 23.49 |
| 541 | HOH | O | 34.56 | 60.04 | 2.17 | 23.60 |
| 542 | HOH | O | 15.22 | 76.50 | −3.73 | 21.84 |
| 543 | HOH | O | 28.57 | 48.62 | 16.32 | 23.52 |
| 545 | HOH | O | 0.12 | 76.37 | 10.30 | 21.07 |
| 547 | HOH | O | 32.52 | 58.58 | 1.80 | 16.49 |
| 549 | HOH | O | 16.82 | 36.91 | 14.38 | 22.51 |
| 550 | HOH | O | 26.17 | 48.07 | 25.22 | 21.54 |
| 551 | HOH | O | 9.79 | 64.32 | −7.51 | 25.56 |
| 552 | HOH | O | 9.13 | 85.46 | 4.86 | 24.49 |
| 554 | HOH | O | 15.79 | 47.89 | 2.91 | 22.12 |
| 556 | HOH | O | 8.92 | 43.02 | 32.25 | 21.25 |
| 557 | HOH | O | 9.33 | 48.39 | −8.70 | 23.77 |
| 559 | HOH | O | 12.66 | 50.32 | 3.58 | 22.02 |
| 560 | HOH | O | 5.50 | 47.13 | −1.19 | 19.70 |
| 561 | HOH | O | 4.21 | 57.58 | 11.07 | 20.67 |
| 562 | HOH | O | −2.10 | 64.46 | −6.00 | 21.42 |
| 565 | HOH | O | −3.58 | 72.97 | 20.53 | 20.33 |
| 566 | HOH | O | 5.15 | 81.64 | 23.84 | 21.73 |
| 567 | HOH | O | 2.86 | 74.92 | 2.91 | 23.52 |
| 568 | HOH | O | −0.26 | 45.98 | 33.72 | 22.01 |
| 569 | HOH | O | 36.81 | 49.98 | −8.54 | 22.28 |
| 570 | HOH | O | −3.48 | 51.99 | 26.44 | 21.30 |
| 571 | HOH | O | 10.85 | 58.69 | 35.02 | 21.96 |
| 574 | HOH | O | 9.58 | 58.70 | 5.62 | 28.90 |
| 575 | HOH | O | 14.88 | 87.03 | 11.84 | 23.94 |
| 576 | HOH | O | 8.51 | 56.74 | 4.55 | 24.46 |
| 577 | HOH | O | 19.14 | 48.40 | −4.28 | 22.63 |
| 578 | HOH | O | 2.29 | 78.38 | 13.61 | 21.50 |
| 579 | HOH | O | 0.95 | 45.84 | 16.79 | 20.93 |
| 580 | HOH | O | −5.15 | 73.68 | 22.71 | 23.34 |
| 581 | HOH | O | 8.86 | 68.48 | −6.29 | 23.28 |
| 582 | HOH | O | 15.97 | 38.02 | 27.28 | 23.18 |
| 585 | HOH | O | 29.59 | 55.27 | 15.87 | 22.11 |
| 587 | HOH | O | 28.98 | 56.33 | 18.23 | 20.66 |
| 588 | HOH | O | 34.65 | 46.54 | −5.61 | 21.69 |
| 590 | HOH | O | 18.54 | 84.32 | 8.80 | 23.61 |
| 593 | HOH | O | 27.01 | 54.68 | 18.19 | 24.38 |
| 601 | HOH | O | 10.69 | 69.18 | 28.99 | 23.32 |
| 602 | HOH | O | 22.51 | 48.74 | −2.80 | 24.21 |
| 603 | HOH | O | −2.73 | 66.02 | 28.47 | 21.85 |
| 604 | HOH | O | −6.55 | 67.81 | 3.05 | 24.57 |
| 605 | HOH | O | 33.48 | 59.82 | 6.28 | 24.71 |
| 606 | HOH | O | 7.93 | 83.52 | 17.49 | 23.44 |
| 610 | HOH | O | 17.19 | 78.89 | −2.32 | 22.39 |
| 611 | HOH | O | 11.32 | 54.82 | −15.30 | 23.51 |
| 614 | HOH | O | −1.82 | 60.34 | 31.64 | 23.08 |
| 615 | HOH | O | 17.79 | 44.50 | 31.66 | 23.97 |
| 616 | HOH | O | 5.95 | 81.58 | 12.22 | 26.87 |
| 617 | HOH | O | 13.30 | 78.15 | −6.87 | 26.85 |
| 618 | HOH | O | 11.07 | 42.45 | 6.02 | 24.18 |
| 619 | HOH | O | 0.40 | 65.16 | 37.45 | 24.44 |
| 620 | HOH | O | 15.44 | 78.74 | 23.41 | 25.05 |
| 621 | HOH | O | 10.15 | 47.78 | 34.39 | 24.19 |
| 622 | HOH | O | 3.65 | 80.72 | 20.28 | 22.24 |
| 623 | HOH | O | 26.64 | 63.48 | −12.01 | 25.94 |
| 625 | HOH | O | 8.66 | 49.94 | −12.65 | 23.77 |
| 626 | HOH | O | 12.07 | 52.23 | −13.98 | 25.56 |
| 627 | HOH | O | 30.91 | 59.44 | 5.61 | 30.73 |
| 628 | HOH | O | 4.72 | 80.56 | 0.69 | 23.63 |
| 629 | HOH | O | 9.84 | 71.51 | −6.15 | 31.12 |
| 633 | HOH | O | 32.67 | 54.55 | 11.08 | 23.40 |
| 634 | HOH | O | 27.25 | 44.92 | 18.37 | 24.08 |
| 635 | HOH | O | 29.55 | 68.33 | 2.13 | 22.95 |
| 636 | HOH | O | 18.86 | 46.30 | 7.18 | 26.04 |
| 638 | HOH | O | 30.69 | 58.96 | −3.48 | 22.96 |
| 640 | HOH | O | 16.51 | 75.33 | 19.53 | 23.27 |
| 642 | HOH | O | 28.96 | 67.43 | 24.75 | 24.99 |
| 643 | HOH | O | 20.47 | 71.46 | 18.92 | 23.03 |
| 644 | HOH | O | 28.36 | 70.87 | 3.05 | 25.69 |
| 645 | HOH | O | 25.19 | 45.77 | 20.02 | 23.64 |
| 649 | HOH | O | −7.68 | 57.11 | 15.91 | 26.16 |
| 650 | HOH | O | 29.29 | 61.88 | 7.51 | 22.66 |
| 651 | HOH | O | 30.34 | 53.30 | −10.77 | 25.90 |
| 652 | HOH | O | 27.63 | 38.11 | 10.37 | 23.80 |
| 653 | HOH | O | 10.15 | 84.70 | 16.36 | 24.69 |
| 654 | HOH | O | 13.06 | 49.74 | 33.82 | 25.88 |
| 655 | HOH | O | −10.35 | 69.12 | 18.18 | 23.77 |
| 656 | HOH | O | −2.08 | 53.04 | 17.47 | 24.18 |
| 657 | HOH | O | 15.41 | 60.75 | 30.17 | 26.79 |
| 658 | HOH | O | 8.21 | 50.70 | 4.12 | 24.45 |
| 659 | HOH | O | 0.64 | 70.35 | 35.28 | 25.78 |
| 660 | HOH | O | 7.70 | 48.53 | −2.38 | 22.91 |
| 661 | HOH | O | 35.00 | 48.28 | −10.01 | 25.41 |
| 662 | HOH | O | 21.69 | 78.93 | 11.71 | 27.63 |
| 663 | HOH | O | 7.60 | 62.42 | −9.91 | 24.53 |
| 664 | HOH | O | 2.98 | 53.47 | 34.59 | 24.15 |
| 665 | HOH | O | 6.18 | 86.10 | 10.47 | 24.56 |
| 666 | HOH | O | 3.06 | 67.19 | −5.27 | 29.34 |
| 667 | HOH | O | −0.03 | 46.12 | 24.07 | 24.93 |
| 668 | HOH | O | 13.02 | 87.37 | 9.86 | 23.55 |
| 669 | HOH | O | 30.13 | 62.84 | 5.13 | 24.61 |
| 670 | HOH | O | 6.73 | 47.03 | −5.41 | 22.73 |
| 671 | HOH | O | 18.24 | 63.72 | 28.40 | 25.21 |
| 672 | HOH | O | 16.13 | 56.69 | −10.76 | 22.10 |
| 673 | HOH | O | 6.04 | 71.82 | 30.96 | 25.26 |
| 674 | HOH | O | −1.28 | 49.98 | 20.87 | 24.08 |
| 675 | HOH | O | 1.59 | 47.59 | 19.12 | 25.26 |
| 676 | HOH | O | 0.62 | 78.05 | 16.15 | 24.35 |
| 677 | HOH | O | 24.64 | 58.08 | 25.78 | 23.95 |
| 678 | HOH | O | 4.19 | 83.63 | 16.78 | 24.64 |
| 679 | HOH | O | −2.76 | 54.35 | 11.79 | 24.22 |
| 680 | HOH | O | 24.94 | 54.22 | 25.04 | 23.55 |
| 681 | HOH | O | −0.92 | 62.44 | 33.08 | 24.41 |
| 683 | HOH | O | 25.95 | 72.80 | 16.14 | 25.24 |
| 684 | HOH | O | 17.82 | 67.65 | 27.64 | 25.87 |
| 685 | HOH | O | 29.21 | 65.94 | 11.73 | 27.58 |
| 686 | HOH | O | −5.11 | 59.90 | 26.63 | 22.58 |
| 687 | HOH | O | 9.53 | 85.35 | 25.14 | 24.70 |
| 688 | HOH | O | 16.93 | 48.52 | −11.69 | 26.84 |
| 689 | HOH | O | −3.73 | 51.75 | 33.39 | 28.01 |
| 690 | HOH | O | 25.99 | 48.39 | 20.23 | 27.48 |
| 691 | HOH | O | 4.93 | 77.99 | 0.20 | 25.82 |
| 692 | HOH | O | −0.98 | 69.53 | 3.23 | 24.46 |
| 693 | HOH | O | 13.06 | 86.70 | 5.50 | 25.80 |
| 694 | HOH | O | −4.12 | 72.37 | 25.16 | 25.50 |
| 695 | HOH | O | 32.03 | 52.55 | 12.83 | 23.94 |
| 696 | HOH | O | 14.62 | 81.94 | −2.30 | 28.15 |
| 697 | HOH | O | 19.13 | 73.67 | 19.87 | 28.03 |
| 698 | HOH | O | 24.41 | 43.07 | 23.62 | 22.94 |
| 699 | HOH | O | −11.38 | 60.83 | 18.91 | 28.63 |
| 700 | HOH | O | 13.81 | 64.17 | 33.23 | 24.40 |
| 701 | HOH | O | −9.26 | 66.77 | 17.67 | 27.19 |
| 702 | HOH | O | 28.65 | 61.97 | −11.14 | 24.05 |
| 703 | HOH | O | −2.19 | 74.92 | 26.01 | 26.63 |
| 704 | HOH | O | −5.43 | 65.69 | 23.69 | 23.47 |
| 705 | HOH | O | 30.72 | 39.51 | 13.13 | 25.31 |
| 706 | HOH | O | −8.68 | 59.80 | 15.17 | 25.88 |
| 707 | HOH | O | 23.82 | 62.58 | 28.39 | 25.61 |
| 708 | HOH | O | 27.55 | 57.95 | 16.86 | 23.71 |
| 709 | HOH | O | 8.98 | 69.48 | 32.94 | 22.81 |
| 710 | HOH | O | 19.95 | 58.71 | 26.08 | 24.64 |
| 711 | HOH | O | 5.40 | 41.87 | 33.09 | 25.37 |
| 712 | HOH | O | 23.80 | 48.54 | −5.14 | 24.53 |
| 713 | HOH | O | 10.60 | 39.95 | 31.40 | 23.19 |
| 714 | HOH | O | 23.01 | 51.94 | 27.26 | 24.37 |
| 715 | HOH | O | 1.74 | 76.11 | 6.68 | 24.61 |
| 716 | HOH | O | 6.08 | 82.67 | 14.55 | 22.35 |
| 717 | HOH | O | 17.99 | 47.36 | 33.21 | 25.38 |
| 718 | HOH | O | 12.29 | 81.85 | 23.22 | 25.39 |
| 719 | HOH | O | 1.58 | 42.75 | 22.30 | 25.49 |
| 720 | HOH | O | 22.80 | 43.93 | 21.05 | 24.55 |
| 721 | HOH | O | 18.57 | 83.82 | 4.79 | 24.51 |
| 722 | HOH | O | 0.85 | 48.37 | 34.99 | 26.02 |
| 723 | HOH | O | 12.21 | 47.49 | −5.30 | 23.82 |
| 724 | HOH | O | 20.23 | 74.06 | 16.07 | 25.19 |
| 725 | HOH | O | −0.67 | 50.96 | 18.33 | 25.39 |
| 726 | HOH | O | 16.94 | 68.40 | −6.42 | 25.90 |
| 727 | HOH | O | 28.39 | 70.52 | 9.63 | 24.39 |
| 728 | HOH | O | 17.39 | 85.88 | 11.12 | 25.86 |
| 729 | HOH | O | 13.81 | 39.41 | 7.60 | 26.16 |
| 730 | HOH | O | 22.58 | 53.60 | −12.80 | 27.37 |
| 731 | HOH | O | 7.50 | 71.39 | −4.97 | 25.83 |
| 732 | HOH | O | 21.27 | 39.86 | 18.56 | 23.57 |
| 733 | HOH | O | 35.67 | 49.44 | −6.15 | 25.91 |
| 734 | HOH | O | 0.08 | 74.63 | 8.21 | 25.05 |
| 735 | HOH | O | 19.20 | 70.45 | 24.90 | 24.94 |
| 736 | HOH | O | 23.39 | 71.70 | 20.20 | 26.92 |
| 738 | HOH | O | 20.65 | 40.15 | 29.15 | 25.47 |
| 739 | HOH | O | −9.06 | 62.29 | 13.08 | 25.76 |
| 740 | HOH | O | 9.75 | 63.89 | −10.10 | 25.02 |
| 741 | HOH | O | 23.32 | 72.22 | 24.80 | 24.88 |
| 742 | HOH | O | 19.61 | 71.77 | 22.62 | 25.68 |
| 743 | HOH | O | −3.41 | 61.18 | 9.75 | 25.55 |
| 744 | HOH | O | 14.06 | 79.57 | −3.48 | 25.35 |
| 745 | HOH | O | 3.63 | 81.35 | 3.06 | 26.94 |
| 746 | HOH | O | −3.66 | 71.16 | 28.13 | 25.25 |
| 747 | HOH | O | 7.29 | 47.30 | 34.13 | 25.37 |
| 748 | HOH | O | 1.82 | 71.84 | 1.20 | 25.66 |
| 749 | HOH | O | 24.45 | 73.71 | −0.14 | 23.90 |
| 750 | HOH | O | 31.78 | 41.85 | 12.32 | 27.58 |
| 751 | HOH | O | −5.89 | 60.50 | 9.29 | 24.52 |
| 752 | HOH | O | 20.34 | 47.90 | 8.55 | 28.66 |
| 753 | HOH | O | 28.19 | 69.18 | 17.09 | 26.56 |
| 754 | HOH | O | 16.62 | 67.85 | −8.93 | 26.55 |
| 755 | HOH | O | 9.22 | 47.99 | −4.54 | 25.91 |
| 756 | HOH | O | 21.68 | 56.70 | 25.60 | 27.54 |
| 757 | HOH | O | 12.55 | 86.43 | 1.99 | 27.42 |
| 758 | HOH | O | 9.95 | 86.42 | 2.30 | 24.86 |
| 759 | HOH | O | 18.95 | 39.77 | 11.68 | 24.89 |
| 760 | HOH | O | 21.34 | 75.46 | −0.39 | 27.65 |
| 761 | HOH | O | −1.90 | 56.40 | 34.01 | 27.94 |
| 762 | HOH | O | −7.87 | 64.96 | 21.91 | 25.75 |
| 763 | HOH | O | 10.29 | 50.48 | 2.35 | 25.43 |
| 764 | HOH | O | −0.94 | 69.76 | 33.06 | 26.40 |
| 765 | HOH | O | −8.20 | 56.03 | 20.28 | 26.25 |
| 766 | HOH | O | 14.68 | 54.76 | 34.57 | 28.83 |
| 767 | HOH | O | −5.50 | 69.51 | 0.99 | 27.56 |
| 768 | HOH | O | −5.77 | 70.12 | 25.30 | 26.53 |
| 769 | HOH | O | 32.18 | 59.01 | −0.64 | 25.31 |
| 770 | HOH | O | 14.91 | 49.39 | −14.49 | 28.33 |
| 771 | HOH | O | 8.47 | 74.57 | −8.39 | 27.05 |
| 772 | HOH | O | 27.30 | 70.51 | 24.96 | 26.24 |
| 773 | HOH | O | 14.18 | 46.00 | −7.72 | 27.27 |
| 774 | HOH | O | 8.16 | 85.03 | 19.99 | 26.88 |
| 775 | HOH | O | 27.31 | 46.33 | 22.79 | 28.48 |
| 776 | HOH | O | 27.10 | 50.17 | 18.22 | 27.70 |
| 777 | HOH | O | 12.86 | 45.30 | 0.54 | 28.03 |
| 778 | HOH | O | 29.05 | 65.07 | 17.18 | 26.29 |
| 779 | HOH | O | 16.18 | 70.09 | 25.83 | 27.45 |
| 780 | HOH | O | 5.77 | 72.80 | 28.61 | 26.22 |
| 781 | HOH | O | 12.57 | 41.90 | 3.75 | 27.47 |
| 782 | HOH | O | 23.72 | 38.29 | 26.52 | 27.15 |
| 783 | HOH | O | 2.62 | 78.60 | 5.08 | 27.48 |
| 784 | HOH | O | 27.24 | 60.99 | 28.04 | 27.30 |
| 785 | HOH | O | 3.09 | 83.56 | 21.96 | 28.25 |
| 786 | HOH | O | 9.54 | 62.42 | 34.93 | 28.70 |
| 787 | HOH | O | −0.21 | 62.57 | 36.04 | 28.47 |
| 788 | HOH | O | 8.47 | 86.82 | 9.12 | 27.38 |
| 789 | HOH | O | 0.57 | 68.11 | −5.92 | 28.59 |
| 790 | HOH | O | 26.45 | 65.68 | −4.66 | 27.55 |
| 791 | HOH | O | 24.48 | 79.51 | 8.63 | 27.10 |
| 792 | HOH | O | 17.35 | 75.25 | 14.78 | 28.54 |
| 793 | HOH | O | 5.48 | 41.85 | 16.75 | 27.99 |
| 794 | HOH | O | 12.58 | 37.82 | 19.90 | 27.74 |
| 795 | HOH | O | 25.21 | 78.73 | 11.98 | 27.98 |
| 796 | HOH | O | 11.04 | 80.21 | −2.38 | 28.78 |
| 299 | HOH | O | 20.33 | 68.86 | 14.90 | 10.41 |
| 290 | HOH | O | 10.01 | 57.54 | 11.33 | 10.75 |
| 291 | HOH | O | 21.46 | 79.00 | 5.63 | 24.93 |
| 292 | HOH | O | −10.74 | 57.08 | 19.29 | 25.97 |
| 293 | HOH | O | 17.81 | 59.62 | 29.37 | 25.06 |
| 294 | HOH | O | −9.38 | 68.28 | 1.29 | 25.84 |
| 295 | HOH | O | −6.46 | 55.28 | 18.30 | 25.89 |
| 296 | HOH | O | 22.37 | 47.06 | 32.44 | 26.10 |
| 297 | HOH | O | 15.31 | 43.97 | 2.12 | 25.16 |
| 298 | HOH | O | 11.20 | 58.31 | 8.87 | 10.62 |
| 287 | ZN2 | ZN | 11.66 | 58.72 | 10.96 | 9.72 |
| 288 | ZN2 | ZN | 10.75 | 55.89 | 12.28 | 13.36 |

TABLE IX
Provides distances between interresidue atoms that are
within 5 Ångstroms apart in an active site of an
S. pneumoniae methionine aminopeptidase.

| Atom 1 | Atom 2 | Distance |
|---|---|---|
| 287ZN | 136OD2 | 1.992 |
| 287ZN | 264OE1 | 2.112 |
| 287ZN | 98OD2 | 2.154 |
| 287ZN | 98OD1 | 2.299 |
| 287ZN | 98CG | 2.539 |
| 287ZN | 136CG | 2.943 |
| 287ZN | 264CD | 2.989 |
| 287ZN | 264OE2 | 3.196 |
| 287ZN | 136OD1 | 3.229 |
| 287ZN | 232OE1 | 4.056 |
| 287ZN | 98CB | 4.059 |
| 287ZN | 137N | 4.137 |
| 287ZN | 138N | 4.218 |
| 287ZN | 136CB | 4.331 |
| 287ZN | 264CG | 4.393 |
| 287ZN | 136C | 4.411 |
| 287ZN | 137C | 4.471 |
| 287ZN | 137CA | 4.505 |
| 287ZN | 138CB | 4.510 |
| 287ZN | 232CD | 4.632 |
| 287ZN | 232OE2 | 4.641 |
| 287ZN | 99O | 4.672 |
| 287ZN | 136CA | 4.710 |
| 287ZN | 138CA | 4.856 |
| 287ZN | 99N | 4.877 |
| 287ZN | 264CB | 4.902 |
| 287ZN | 100CG2 | 4.928 |
| 287ZN | 98CA | 4.957 |
| 287ZN | 136O | 4.962 |
| 136OD2 | 287ZN | 90.61 |
| 136OD2 | 287ZN | 150.95 |
| 136OD2 | 287ZN | 92.52 |
| 136OD2 | 287ZN | 121.53 |
| 136OD2 | 287ZN | 19.04 |
| 136OD2 | 287ZN | 79.83 |
| 136OD2 | 287ZN | 72.33 |
| 136OD2 | 287ZN | 41.87 |
| 136OD2 | 287ZN | 125.43 |
| 136OD2 | 287ZN | 122.88 |
| 136OD2 | 287ZN | 41.43 |
| 136OD2 | 287ZN | 90.24 |
| 136OD2 | 287ZN | 9.33 |
| 136OD2 | 287ZN | 81.14 |
| 136OD2 | 287ZN | 27.39 |
| 136OD2 | 287ZN | 75.32 |
| 136OD2 | 287ZN | 58.50 |
| 136OD2 | 287ZN | 122.59 |
| 136OD2 | 287ZN | 111.26 |
| 136OD2 | 287ZN | 97.20 |
| 136OD2 | 287ZN | 49.76 |
| 136OD2 | 287ZN | 10.22 |
| 136OD2 | 287ZN | 105.29 |
| 136OD2 | 287ZN | 82.28 |
| 136OD2 | 287ZN | 95.97 |
| 136OD2 | 287ZN | 49.30 |
| 136OD2 | 287ZN | 107.66 |
| 136OD2 | 287ZN | 33.80 |
| 264OE1 | 287ZN | 96.24 |
| 264OE1 | 287ZN | 96.44 |
| 264OE1 | 287ZN | 99.57 |
| 264OE1 | 287ZN | 92.65 |
| 264OE1 | 287ZN | 20.50 |
| 264OE1 | 287ZN | 43.43 |
| 264OE1 | 287ZN | 95.93 |
| 264OE1 | 287ZN | 68.59 |
| 264OE1 | 287ZN | 102.46 |
| 264OE1 | 287ZN | 67.80 |
| 264OE1 | 287ZN | 45.65 |
| 264OE1 | 287ZN | 90.60 |
| 264OE1 | 287ZN | 12.68 |
| 264OE1 | 287ZN | 68.51 |
| 264OE1 | 287ZN | 55.97 |
| 264OE1 | 287ZN | 52.32 |
| 264OE1 | 287ZN | 53.79 |
| 264OE1 | 287ZN | 61.47 |
| 264OE1 | 287ZN | 66.31 |
| 264OE1 | 287ZN | 110.16 |
| 264OE1 | 287ZN | 86.04 |
| 264OE1 | 287ZN | 54.03 |
| 264OE1 | 287ZN | 112.81 |
| 264OE1 | 287ZN | 5.53 |
| 264OE1 | 287ZN | 139.48 |
| 264OE1 | 287ZN | 99.05 |
| 264OE1 | 287ZN | 57.60 |
| 98OD2 | 287ZN | 58.71 |
| 98OD2 | 287ZN | 29.46 |
| 98OD2 | 287ZN | 167.11 |
| 98OD2 | 287ZN | 113.88 |
| 98OD2 | 287ZN | 129.80 |
| 98OD2 | 287ZN | 161.97 |
| 98OD2 | 287ZN | 83.12 |
| 98OD2 | 287ZN | 28.08 |
| 98OD2 | 287ZN | 116.60 |
| 98OD2 | 287ZN | 75.05 |
| 98OD2 | 287ZN | 159.88 |
| 98OD2 | 287ZN | 108.57 |
| 98OD2 | 287ZN | 134.07 |
| 98OD2 | 287ZN | 85.25 |
| 98OD2 | 287ZN | 104.61 |
| 98OD2 | 287ZN | 46.58 |
| 98OD2 | 287ZN | 96.72 |
| 98OD2 | 287ZN | 111.42 |
| 98OD2 | 287ZN | 101.67 |
| 98OD2 | 287ZN | 142.66 |
| 98OD2 | 287ZN | 58.47 |
| 98OD2 | 287ZN | 69.02 |
| 98OD2 | 287ZN | 90.87 |
| 98OD2 | 287ZN | 116.16 |
| 98OD2 | 287ZN | 43.39 |
| 98OD2 | 287ZN | 137.53 |
| 98OD1 | 287ZN | 29.42 |
| 98OD1 | 287ZN | 111.06 |
| 98OD1 | 287ZN | 114.07 |
| 98OD1 | 287ZN | 134.75 |
| 98OD1 | 287ZN | 132.67 |
| 98OD1 | 287ZN | 137.83 |
| 98OD1 | 287ZN | 31.47 |
| 98OD1 | 287ZN | 62.80 |
| 98OD1 | 287ZN | 50.86 |
| 98OD1 | 287ZN | 101.79 |
| 98OD1 | 287ZN | 105.15 |
| 98OD1 | 287ZN | 79.51 |
| 98OD1 | 287ZN | 45.02 |
| 98OD1 | 287ZN | 60.54 |
| 98OD1 | 287ZN | 55.97 |
| 98OD1 | 287ZN | 146.52 |
| 98OD1 | 287ZN | 160.16 |
| 98OD1 | 287ZN | 46.43 |
| 98OD1 | 287ZN | 83.96 |
| 98OD1 | 287ZN | 44.98 |
| 98OD1 | 287ZN | 19.36 |
| 98OD1 | 287ZN | 94.81 |
| 98OD1 | 287ZN | 81.89 |
| 98OD1 | 287ZN | 15.47 |
| 98OD1 | 287ZN | 89.20 |
| 98CG | 287ZN | 139.36 |
| 98CG | 287ZN | 120.07 |
| 98CG | 287ZN | 142.73 |
| 98CG | 287ZN | 157.60 |
| 98CG | 287ZN | 111.67 |
| 98CG | 287ZN | 3.38 |
| 98CG | 287ZN | 90.56 |
| 98CG | 287ZN | 61.38 |
| 98CG | 287ZN | 130.65 |
| 98CG | 287ZN | 111.71 |
| 98CG | 287ZN | 107.90 |
| 98CG | 287ZN | 64.66 |
| 98CG | 287ZN | 83.55 |
| 98CG | 287ZN | 46.58 |
| 98CG | 287ZN | 124.18 |
| 98CG | 287ZN | 139.55 |
| 98CG | 287ZN | 73.04 |
| 98CG | 287ZN | 113.33 |
| 98CG | 287ZN | 47.31 |
| 98CG | 287ZN | 40.72 |
| 98CG | 287ZN | 95.54 |
| 98CG | 287ZN | 98.06 |
| 98CG | 287ZN | 13.98 |
| 98CG | 287ZN | 115.73 |
| 136CG | 287ZN | 76.76 |
| 136CG | 287ZN | 62.66 |
| 136CG | 287ZN | 22.84 |
| 136CG | 287ZN | 108.89 |
| 136CG | 287ZN | 140.16 |
| 136CG | 287ZN | 58.63 |
| 136CG | 287ZN | 105.13 |
| 136CG | 287ZN | 9.82 |
| 136CG | 287ZN | 80.95 |
| 136CG | 287ZN | 42.30 |
| 136CG | 287ZN | 92.06 |
| 136CG | 287ZN | 73.70 |
| 136CG | 287ZN | 136.63 |
| 136CG | 287ZN | 95.72 |
| 136CG | 287ZN | 80.72 |
| 136CG | 287ZN | 66.37 |
| 136CG | 287ZN | 28.84 |
| 136CG | 287ZN | 121.28 |
| 136CG | 287ZN | 98.91 |
| 136CG | 287ZN | 98.16 |
| 136CG | 287ZN | 51.97 |
| 136CG | 287ZN | 125.84 |
| 136CG | 287ZN | 43.17 |
| 264CD | 287ZN | 23.07 |
| 264CD | 287ZN | 76.24 |
| 264CD | 287ZN | 63.17 |
| 264CD | 287ZN | 122.95 |
| 264CD | 287ZN | 70.41 |
| 264CD | 287ZN | 63.68 |
| 264CD | 287ZN | 77.16 |
| 264CD | 287ZN | 9.03 |
| 264CD | 287ZN | 64.48 |
| 264CD | 287ZN | 70.17 |
| 264CD | 287ZN | 60.22 |
| 264CD | 287ZN | 74.08 |
| 264CD | 287ZN | 51.78 |
| 264CD | 287ZN | 51.48 |
| 264CD | 287ZN | 113.95 |
| 264CD | 287ZN | 78.38 |
| 264CD | 287ZN | 74.06 |
| 264CD | 287ZN | 127.54 |
| 264CD | 287ZN | 24.80 |
| 264CD | 287ZN | 128.11 |
| 264CD | 287ZN | 118.87 |
| 264CD | 287ZN | 51.35 |
| 264OE2 | 287ZN | 55.85 |
| 264OE2 | 287ZN | 57.71 |
| 264OE2 | 287ZN | 145.37 |
| 264OE2 | 287ZN | 79.65 |
| 264OE2 | 287ZN | 85.96 |
| 264OE2 | 287ZN | 66.49 |
| 264OE2 | 287ZN | 31.91 |
| 264OE2 | 287ZN | 67.60 |
| 264OE2 | 287ZN | 89.73 |
| 264OE2 | 287ZN | 75.65 |
| 264OE2 | 287ZN | 96.22 |
| 264OE2 | 287ZN | 43.32 |
| 264OE2 | 287ZN | 35.86 |
| 264OE2 | 287ZN | 118.03 |
| 264OE2 | 287ZN | 74.96 |
| 264OE2 | 287ZN | 97.11 |
| 264OE2 | 287ZN | 143.16 |
| 264OE2 | 287ZN | 47.22 |
| 264OE2 | 287ZN | 113.83 |
| 264OE2 | 287ZN | 141.71 |
| 264OE2 | 287ZN | 54.51 |
| 136OD1 | 287ZN | 89.01 |
| 136OD1 | 287ZN | 156.67 |
| 136OD1 | 287ZN | 80.41 |
| 136OD1 | 287ZN | 122.82 |
| 136OD1 | 287ZN | 32.66 |
| 136OD1 | 287ZN | 83.25 |
| 136OD1 | 287ZN | 63.25 |
| 136OD1 | 287ZN | 112.63 |
| 136OD1 | 287ZN | 93.36 |
| 136OD1 | 287ZN | 149.02 |
| 136OD1 | 287ZN | 77.59 |
| 136OD1 | 287ZN | 62.09 |
| 136OD1 | 287ZN | 86.50 |
| 136OD1 | 287ZN | 51.64 |
| 136OD1 | 287ZN | 139.37 |
| 136OD1 | 287ZN | 117.77 |
| 136OD1 | 287ZN | 100.83 |
| 136OD1 | 287ZN | 59.94 |
| 136OD1 | 287ZN | 146.21 |
| 136OD1 | 287ZN | 60.50 |
| 232OE1 | 287ZN | 110.94 |
| 232OE1 | 287ZN | 133.58 |
| 232OE1 | 287ZN | 105.65 |
| 232OE1 | 287ZN | 116.96 |
| 232OE1 | 287ZN | 68.41 |
| 232OE1 | 287ZN | 125.02 |
| 232OE1 | 287ZN | 121.39 |
| 232OE1 | 287ZN | 120.80 |
| 232OE1 | 287ZN | 85.49 |
| 232OE1 | 287ZN | 14.69 |
| 232OE1 | 287ZN | 28.30 |
| 232OE1 | 287ZN | 175.19 |
| 232OE1 | 287ZN | 131.14 |
| 232OE1 | 287ZN | 101.45 |
| 232OE1 | 287ZN | 152.14 |
| 232OE1 | 287ZN | 66.44 |
| 232OE1 | 287ZN | 135.31 |
| 232OE1 | 287ZN | 124.67 |
| 232OE1 | 287ZN | 111.63 |
| 98CB | 287ZN | 93.28 |
| 98CB | 287ZN | 64.75 |
| 98CB | 287ZN | 131.84 |
| 98CB | 287ZN | 114.70 |
| 98CB | 287ZN | 110.50 |
| 98CB | 287ZN | 67.95 |
| 98CB | 287ZN | 86.76 |
| 98CB | 287ZN | 49.16 |
| 98CB | 287ZN | 123.94 |
| 98CB | 287ZN | 139.11 |
| 98CB | 287ZN | 73.82 |
| 98CB | 287ZN | 115.05 |
| 98CB | 287ZN | 50.57 |
| 98CB | 287ZN | 41.25 |
| 98CB | 287ZN | 98.31 |
| 98CB | 287ZN | 96.77 |
| 98CB | 287ZN | 16.02 |
| 98CB | 287ZN | 118.72 |
| 137N | 287ZN | 49.38 |
| 137N | 287ZN | 49.25 |
| 137N | 287ZN | 65.52 |
| 137N | 287ZN | 17.56 |
| 137N | 287ZN | 33.89 |
| 137N | 287ZN | 18.89 |
| 137N | 287ZN | 81.50 |
| 137N | 287ZN | 121.35 |
| 137N | 287ZN | 115.30 |
| 137N | 287ZN | 43.79 |
| 137N | 287ZN | 31.37 |
| 137N | 287ZN | 63.88 |
| 137N | 287ZN | 63.76 |
| 137N | 287ZN | 71.43 |
| 137N | 287ZN | 75.86 |
| 137N | 287ZN | 77.57 |
| 137N | 287ZN | 26.64 |
| 138N | 287ZN | 96.89 |
| 138N | 287ZN | 54.65 |
| 138N | 287ZN | 63.33 |
| 138N | 287ZN | 17.28 |
| 138N | 287ZN | 31.79 |
| 138N | 287ZN | 32.35 |
| 138N | 287ZN | 103.95 |
| 138N | 287ZN | 111.63 |
| 138N | 287ZN | 75.48 |
| 138N | 287ZN | 80.51 |
| 138N | 287ZN | 16.59 |
| 138N | 287ZN | 67.54 |
| 138N | 287ZN | 44.56 |
| 138N | 287ZN | 117.91 |
| 138N | 287ZN | 55.90 |
| 138N | 287ZN | 62.74 |
| 136CB | 287ZN | 79.89 |
| 136CB | 287ZN | 33.58 |
| 136CB | 287ZN | 82.96 |
| 136CB | 287ZN | 65.16 |
| 136CB | 287ZN | 129.03 |
| 136CB | 287ZN | 103.16 |
| 136CB | 287ZN | 88.66 |
| 136CB | 287ZN | 58.24 |
| 136CB | 287ZN | 19.02 |
| 136CB | 287ZN | 112.62 |
| 136CB | 287ZN | 90.88 |
| 136CB | 287ZN | 96.11 |
| 136CB | 287ZN | 50.97 |
| 136CB | 287ZN | 116.85 |
| 136CB | 287ZN | 36.70 |
| 264CG | 287ZN | 62.27 |
| 264CG | 287ZN | 61.70 |
| 264CG | 287ZN | 53.30 |
| 264CG | 287ZN | 66.38 |
| 264CG | 287ZN | 58.39 |
| 264CG | 287ZN | 59.72 |
| 264CG | 287ZN | 109.29 |
| 264CG | 287ZN | 78.10 |
| 264CG | 287ZN | 65.22 |
| 264CG | 287ZN | 119.31 |
| 264CG | 287ZN | 17.77 |
| 264CG | 287ZN | 130.41 |
| 264CG | 287ZN | 109.90 |
| 264CG | 287ZN | 49.92 |
| 136C | 287ZN | 49.81 |
| 136C | 287ZN | 31.58 |
| 136C | 287ZN | 95.59 |
| 136C | 287ZN | 110.90 |
| 136C | 287ZN | 101.64 |
| 136C | 287ZN | 51.08 |
| 136C | 287ZN | 18.96 |
| 136C | 287ZN | 79.10 |
| 136C | 287ZN | 77.51 |
| 136C | 287ZN | 73.28 |
| 136C | 287ZN | 71.42 |
| 136C | 287ZN | 94.62 |
| 136C | 287ZN | 13.48 |
| 137C | 287ZN | 19.50 |
| 137C | 287ZN | 47.95 |
| 137C | 287ZN | 117.20 |
| 137C | 287ZN | 121.40 |
| 137C | 287ZN | 59.16 |
| 137C | 287ZN | 65.23 |
| 137C | 287ZN | 30.03 |
| 137C | 287ZN | 57.65 |
| 137C | 287ZN | 56.53 |
| 137C | 287ZN | 101.01 |
| 137C | 287ZN | 55.11 |
| 137C | 287ZN | 52.73 |
| 137CA | 287ZN | 64.12 |
| 137CA | 287ZN | 111.69 |
| 137CA | 287ZN | 110.66 |
| 137CA | 287ZN | 57.85 |
| 137CA | 287ZN | 48.96 |
| 137CA | 287ZN | 47.59 |
| 137CA | 287ZN | 68.34 |
| 137CA | 287ZN | 55.04 |
| 137CA | 287ZN | 94.22 |
| 137CA | 287ZN | 72.83 |
| 137CA | 287ZN | 33.23 |
| 138CB | 287ZN | 90.81 |
| 138CB | 287ZN | 104.39 |
| 138CB | 287ZN | 97.50 |
| 138CB | 287ZN | 112.79 |
| 138CB | 287ZN | 18.33 |
| 138CB | 287ZN | 75.23 |
| 138CB | 287ZN | 49.30 |
| 138CB | 287ZN | 137.74 |
| 138CB | 287ZN | 50.29 |
| 138CB | 287ZN | 93.47 |
| 232CD | 287ZN | 15.49 |
| 232CD | 287ZN | 160.67 |
| 232CD | 287ZN | 116.51 |
| 232CD | 287ZN | 104.07 |
| 232CD | 287ZN | 164.60 |
| 232CD | 287ZN | 60.67 |
| 232CD | 287ZN | 131.45 |
| 232CD | 287ZN | 136.04 |
| 232CD | 287ZN | 97.68 |
| 232OE2 | 287ZN | 146.90 |
| 232OE2 | 287ZN | 103.62 |
| 232OE2 | 287ZN | 115.42 |
| 232OE2 | 287ZN | 179.00 |
| 232OE2 | 287ZN | 67.06 |
| 232OE2 | 287ZN | 117.46 |
| 232OE2 | 287ZN | 151.49 |
| 232OE2 | 287ZN | 89.33 |
| 99O | 287ZN | 44.16 |
| 99O | 287ZN | 80.94 |
| 99O | 287ZN | 32.65 |
| 99O | 287ZN | 112.75 |
| 99O | 287ZN | 42.76 |
| 99O | 287ZN | 59.92 |
| 99O | 287ZN | 64.54 |
| 136CA | 287ZN | 95.25 |
| 136CA | 287ZN | 75.75 |
| 136CA | 287ZN | 91.12 |
| 136CA | 287ZN | 53.46 |
| 136CA | 287ZN | 99.35 |
| 136CA | 287ZN | 28.47 |
| 138CA | 287ZN | 63.97 |
| 138CA | 287ZN | 51.18 |
| 138CA | 287ZN | 123.15 |
| 138CA | 287ZN | 45.03 |
| 138CA | 287ZN | 79.30 |
| 99N | 287ZN | 112.11 |
| 99N | 287ZN | 62.83 |
| 99N | 287ZN | 28.56 |
| 99N | 287ZN | 89.76 |
| 264CB | 287ZN | 144.56 |
| 264CB | 287ZN | 96.08 |
| 264CB | 287ZN | 62.75 |
| 100CG2 | 287ZN | 89.80 |
| 100CG2 | 287ZN | 81.88 |
| 98CA | 287ZN | 103.52 |
| 288ZN | 264OE2 | 2.079 |
| 288ZN | 136OD1 | 2.086 |
| 288ZN | 199NE2 | 2.106 |
| 288ZN | 232OE2 | 2.172 |
| 288ZN | 232CD | 3.007 |
| 288ZN | 136CG | 3.008 |
| 288ZN | 199CE1 | 3.032 |
| 288ZN | 199CD2 | 3.100 |
| 288ZN | 264CD | 3.134 |
| 288ZN | 232OE1 | 3.261 |
| 288ZN | 136OD2 | 3.375 |
| 288ZN | 264OE1 | 3.542 |
| 288ZN | 230OG1 | 3.546 |
| 288ZN | 230CG2 | 3.892 |
| 288ZN | 230CB | 3.972 |
| 288ZN | 199ND1 | 4.140 |
| 288ZN | 199CG | 4.205 |
| 288ZN | 136CB | 4.268 |
| 288ZN | 232CG | 4.328 |
| 288ZN | 264CG | 4.437 |
| 288ZN | 232CB | 4.898 |
| 288ZN | 98OD2 | 4.981 |
| 264OE2 | 288ZN | 92.52 |
| 264OE2 | 288ZN | 135.97 |
| 264OE2 | 288ZN | 81.54 |
| 264OE2 | 288ZN | 75.02 |
| 264OE2 | 288ZN | 75.50 |
| 264OE2 | 288ZN | 124.12 |
| 264OE2 | 288ZN | 132.36 |
| 264OE2 | 288ZN | 15.25 |
| 264OE2 | 288ZN | 80.90 |
| 264OE2 | 288ZN | 67.40 |
| 264OE2 | 288ZN | 35.47 |
| 264OE2 | 288ZN | 72.15 |
| 264OE2 | 288ZN | 63.00 |
| 264OE2 | 288ZN | 56.16 |
| 264OE2 | 288ZN | 124.97 |
| 264OE2 | 288ZN | 128.67 |
| 264OE2 | 288ZN | 74.97 |
| 264OE2 | 288ZN | 66.62 |
| 264OE2 | 288ZN | 6.40 |
| 264OE2 | 288ZN | 50.24 |
| 264OE2 | 288ZN | 74.73 |
| 136OD1 | 288ZN | 89.61 |
| 136OD1 | 288ZN | 168.18 |
| 136OD1 | 288ZN | 164.05 |
| 136OD1 | 288ZN | 19.54 |
| 136OD1 | 288ZN | 70.81 |
| 136OD1 | 288ZN | 110.92 |
| 136OD1 | 288ZN | 92.63 |
| 136OD1 | 288ZN | 147.18 |
| 136OD1 | 288ZN | 39.10 |
| 136OD1 | 288ZN | 87.68 |
| 136OD1 | 288ZN | 113.34 |
| 136OD1 | 288ZN | 76.96 |
| 136OD1 | 288ZN | 98.46 |
| 136OD1 | 288ZN | 82.45 |
| 136OD1 | 288ZN | 100.72 |
| 136OD1 | 288ZN | 18.07 |
| 136OD1 | 288ZN | 159.10 |
| 136OD1 | 288ZN | 97.29 |
| 136OD1 | 288ZN | 141.81 |
| 136OD1 | 288ZN | 87.62 |
| 199NE2 | 288ZN | 87.65 |
| 199NE2 | 288ZN | 106.25 |
| 199NE2 | 288ZN | 108.47 |
| 199NE2 | 288ZN | 21.48 |
| 199NE2 | 288ZN | 21.37 |
| 199NE2 | 288ZN | 151.20 |
| 199NE2 | 288ZN | 117.38 |
| 199NE2 | 288ZN | 128.57 |
| 199NE2 | 288ZN | 170.75 |
| 199NE2 | 288ZN | 66.77 |
| 199NE2 | 288ZN | 74.80 |
| 199NE2 | 288ZN | 80.02 |
| 199NE2 | 288ZN | 13.14 |
| 199NE2 | 288ZN | 12.54 |
| 199NE2 | 288ZN | 99.19 |
| 199NE2 | 288ZN | 106.24 |
| 199NE2 | 288ZN | 139.81 |
| 199NE2 | 288ZN | 121.87 |
| 199NE2 | 288ZN | 149.29 |
| 232OE2 | 288ZN | 20.97 |
| 232OE2 | 288ZN | 157.04 |
| 232OE2 | 288ZN | 104.05 |
| 232OE2 | 288ZN | 67.12 |
| 232OE2 | 288ZN | 84.34 |
| 232OE2 | 288ZN | 42.10 |
| 232OE2 | 288ZN | 143.05 |
| 232OE2 | 288ZN | 93.22 |
| 232OE2 | 288ZN | 55.14 |
| 232OE2 | 288ZN | 91.22 |
| 232OE2 | 288ZN | 69.74 |
| 232OE2 | 288ZN | 92.54 |
| 232OE2 | 288ZN | 75.60 |
| 232OE2 | 288ZN | 152.41 |
| 232OE2 | 288ZN | 18.73 |
| 232OE2 | 288ZN | 77.64 |
| 232OE2 | 288ZN | 35.89 |
| 232OE2 | 288ZN | 100.54 |
| 232CD | 288ZN | 144.63 |
| 232CD | 288ZN | 124.38 |
| 232CD | 288ZN | 84.98 |
| 232CD | 288ZN | 72.72 |
| 232CD | 288ZN | 22.53 |
| 232CD | 288ZN | 125.15 |
| 232CD | 288ZN | 76.40 |
| 232CD | 288ZN | 72.79 |
| 232CD | 288ZN | 105.15 |
| 232CD | 288ZN | 83.02 |
| 232CD | 288ZN | 112.75 |
| 232CD | 288ZN | 94.89 |
| 232CD | 288ZN | 149.46 |
| 232CD | 288ZN | 11.65 |
| 232CD | 288ZN | 69.60 |
| 232CD | 288ZN | 24.79 |
| 232CD | 288ZN | 79.59 |
| 136CG | 288ZN | 88.59 |
| 136CG | 288ZN | 129.84 |
| 136CG | 288ZN | 73.67 |
| 136CG | 288ZN | 132.11 |
| 136CG | 288ZN | 21.49 |
| 136CG | 288ZN | 68.25 |
| 136CG | 288ZN | 115.51 |
| 136CG | 288ZN | 77.94 |
| 136CG | 288ZN | 96.39 |
| 136CG | 288ZN | 100.04 |
| 136CG | 288ZN | 118.86 |
| 136CG | 288ZN | 13.55 |
| 136CG | 288ZN | 140.57 |
| 136CG | 288ZN | 79.55 |
| 136CG | 288ZN | 122.91 |
| 136CG | 288ZN | 74.13 |
| 199CE1 | 288ZN | 41.86 |
| 199CE1 | 288ZN | 138.14 |
| 199CE1 | 288ZN | 138.52 |
| 199CE1 | 288ZN | 109.70 |
| 199CE1 | 288ZN | 151.22 |
| 199CE1 | 288ZN | 67.52 |
| 199CE1 | 288ZN | 61.35 |
| 199CE1 | 288ZN | 73.57 |
| 199CE1 | 288ZN | 11.65 |
| 199CE1 | 288ZN | 30.28 |
| 199CE1 | 288ZN | 78.25 |
| 199CE1 | 288ZN | 121.38 |
| 199CE1 | 288ZN | 129.78 |
| 199CE1 | 288ZN | 133.31 |
| 199CE1 | 288ZN | 150.83 |
| 199CD2 | 288ZN | 144.52 |
| 199CD2 | 288ZN | 96.67 |
| 199CD2 | 288ZN | 149.65 |
| 199CD2 | 288ZN | 160.11 |
| 199CD2 | 288ZN | 60.64 |
| 199CD2 | 288ZN | 82.07 |
| 199CD2 | 288ZN | 79.08 |
| 199CD2 | 288ZN | 31.07 |
| 199CD2 | 288ZN | 12.47 |
| 199CD2 | 288ZN | 120.11 |
| 199CD2 | 288ZN | 85.85 |
| 199CD2 | 288ZN | 133.15 |
| 199CD2 | 288ZN | 102.48 |
| 199CD2 | 288ZN | 143.55 |
| 264CD | 288ZN | 72.89 |
| 264CD | 288ZN | 60.52 |
| 264CD | 288ZN | 20.48 |
| 264CD | 288ZN | 86.11 |
| 264CD | 288ZN | 77.76 |
| 264CD | 288ZN | 71.24 |
| 264CD | 288ZN | 140.08 |
| 264CD | 288ZN | 143.10 |
| 264CD | 288ZN | 76.83 |
| 264CD | 288ZN | 66.90 |
| 264CD | 288ZN | 11.89 |
| 264CD | 288ZN | 49.27 |
| 264CD | 288ZN | 59.51 |
| 232OE1 | 288ZN | 110.63 |
| 232OE1 | 288ZN | 68.35 |
| 232OE1 | 288ZN | 95.30 |
| 232OE1 | 288ZN | 125.85 |
| 232OE1 | 288ZN | 104.10 |
| 232OE1 | 288ZN | 127.48 |
| 232OE1 | 288ZN | 108.49 |
| 232OE1 | 288ZN | 143.21 |
| 232OE1 | 288ZN | 32.73 |
| 232OE1 | 288ZN | 74.59 |
| 232OE1 | 288ZN | 36.87 |
| 232OE1 | 288ZN | 59.59 |
| 136OD2 | 288ZN | 49.85 |
| 136OD2 | 288ZN | 126.65 |
| 136OD2 | 288ZN | 91.95 |
| 136OD2 | 288ZN | 105.70 |
| 136OD2 | 288ZN | 121.27 |
| 136OD2 | 288ZN | 139.80 |
| 136OD2 | 288ZN | 33.93 |
| 136OD2 | 288ZN | 124.35 |
| 136OD2 | 288ZN | 69.46 |
| 136OD2 | 288ZN | 107.29 |
| 136OD2 | 288ZN | 53.31 |
| 264OE1 | 288ZN | 106.31 |
| 264OE1 | 288ZN | 95.97 |
| 264OE1 | 288ZN | 91.63 |
| 264OE1 | 288ZN | 157.82 |
| 264OE1 | 288ZN | 163.17 |
| 264OE1 | 288ZN | 75.94 |
| 264OE1 | 288ZN | 74.50 |
| 264OE1 | 288ZN | 32.27 |
| 264OE1 | 288ZN | 57.66 |
| 264OE1 | 288ZN | 39.41 |
| 230OG1 | 288ZN | 37.69 |
| 230OG1 | 288ZN | 21.01 |
| 230OG1 | 288ZN | 60.54 |
| 230OG1 | 288ZN | 57.00 |
| 230OG1 | 288ZN | 103.06 |
| 230OG1 | 288ZN | 63.16 |
| 230OG1 | 288ZN | 74.22 |
| 230OG1 | 288ZN | 67.83 |
| 230OG1 | 288ZN | 141.17 |
| 230CG2 | 288ZN | 22.15 |
| 230CG2 | 288ZN | 62.50 |
| 230CG2 | 288ZN | 72.14 |
| 230CG2 | 288ZN | 65.36 |
| 230CG2 | 288ZN | 93.81 |
| 230CG2 | 288ZN | 68.45 |
| 230CG2 | 288ZN | 90.19 |
| 230CG2 | 288ZN | 133.81 |
| 230CB | 288ZN | 70.40 |
| 230CB | 288ZN | 72.83 |
| 230CB | 288ZN | 85.06 |
| 230CB | 288ZN | 71.75 |
| 230CB | 288ZN | 59.82 |
| 230CB | 288ZN | 69.74 |
| 230CB | 288ZN | 130.63 |
| 199ND1 | 288ZN | 18.99 |
| 199ND1 | 288ZN | 89.26 |
| 199ND1 | 288ZN | 110.17 |
| 199ND1 | 288ZN | 129.68 |
| 199ND1 | 288ZN | 123.28 |
| 199ND1 | 288ZN | 158.13 |
| 199CG | 288ZN | 108.25 |
| 199CG | 288ZN | 94.03 |
| 199CG | 288ZN | 131.21 |
| 199CG | 288ZN | 109.36 |
| 199CG | 288ZN | 154.06 |
| 136CB | 288ZN | 141.50 |
| 136CB | 288ZN | 80.09 |
| 136CB | 288ZN | 125.01 |
| 136CB | 288ZN | 87.17 |
| 232CG | 288ZN | 61.80 |
| 232CG | 288ZN | 17.67 |
| 232CG | 288ZN | 85.44 |
| 264CG | 288ZN | 44.93 |
| 264CG | 288ZN | 70.81 |
| 232CB | 288ZN | 75.59 |

TABLE X
Provides angles between interresidue atoms that are
within 5 Ångstroms apart in an active site of an
S. pneumoniae methionine aminopeptidase.

| Atom 1 | Atom 2 | Atom 3 | Angle |
|---|---|---|---|
| 136OD2 | 287ZN | 264OE1 | 90.61 |
| 136OD2 | 287ZN | 98OD1 | 92.52 |
| 136OD2 | 287ZN | 136CG | 19.04 |
| 136OD2 | 287ZN | 264CD | 79.83 |
| 136OD2 | 287ZN | 264OE2 | 72.33 |
| 136OD2 | 287ZN | 136OD1 | 41.87 |
| 136OD2 | 287ZN | 137N | 41.43 |
| 136OD2 | 287ZN | 138N | 90.24 |
| 136OD2 | 287ZN | 136CB | 9.33 |
| 136OD2 | 287ZN | 264CG | 81.14 |
| 136OD2 | 287ZN | 136C | 27.39 |
| 136OD2 | 287ZN | 137C | 75.32 |
| 136OD2 | 287ZN | 137CA | 58.50 |
| 136OD2 | 287ZN | 232OE2 | 97.20 |
| 136OD2 | 287ZN | 99O | 49.76 |
| 136OD2 | 287ZN | 136CA | 10.22 |
| 136OD2 | 287ZN | 99N | 82.28 |
| 136OD2 | 287ZN | 264CB | 95.97 |
| 136OD2 | 287ZN | 100CG2 | 49.30 |
| 136OD2 | 287ZN | 136O | 33.80 |
| 264OE1 | 287ZN | 98OD2 | 96.24 |
| 264OE1 | 287ZN | 98OD1 | 96.44 |
| 264OE1 | 287ZN | 98CG | 99.57 |
| 264OE1 | 287ZN | 136CG | 92.65 |
| 264OE1 | 287ZN | 264CD | 20.50 |
| 264OE1 | 287ZN | 264OE2 | 43.43 |
| 264OE1 | 287ZN | 136OD1 | 95.93 |
| 264OE1 | 287ZN | 232OE1 | 68.59 |
| 264OE1 | 287ZN | 137N | 67.80 |
| 264OE1 | 287ZN | 138N | 45.65 |
| 264OE1 | 287ZN | 136CB | 90.60 |
| 264OE1 | 287ZN | 264CG | 12.68 |
| 264OE1 | 287ZN | 136C | 68.51 |
| 264OE1 | 287ZN | 137C | 55.97 |
| 264OE1 | 287ZN | 137CA | 52.32 |
| 264OE1 | 287ZN | 138CB | 53.79 |
| 264OE1 | 287ZN | 232CD | 61.47 |
| 264OE1 | 287ZN | 232OE2 | 66.31 |
| 264OE1 | 287ZN | 136CA | 86.04 |
| 264OE1 | 287ZN | 138CA | 54.03 |
| 264OE1 | 287ZN | 264CB | 5.53 |
| 264OE1 | 287ZN | 98CA | 99.05 |
| 264OE1 | 287ZN | 136O | 57.60 |
| 98OD2 | 287ZN | 98OD1 | 58.71 |
| 98OD2 | 287ZN | 98CG | 29.46 |
| 98OD2 | 287ZN | 232OE1 | 83.12 |
| 98OD2 | 287ZN | 98CB | 28.08 |
| 98OD2 | 287ZN | 138N | 75.05 |
| 98OD2 | 287ZN | 137C | 85.25 |
| 98OD2 | 287ZN | 138CB | 46.58 |
| 98OD2 | 287ZN | 232CD | 96.72 |
| 98OD2 | 287ZN | 138CA | 58.47 |
| 98OD2 | 287ZN | 99N | 69.02 |
| 98OD2 | 287ZN | 264CB | 90.87 |
| 98OD2 | 287ZN | 98CA | 43.39 |
| 98OD1 | 287ZN | 98CG | 29.42 |
| 98OD1 | 287ZN | 98CB | 31.47 |
| 98OD1 | 287ZN | 137N | 62.80 |
| 98OD1 | 287ZN | 138N | 50.86 |
| 98OD1 | 287ZN | 136C | 79.51 |
| 98OD1 | 287ZN | 137C | 45.02 |
| 98OD1 | 287ZN | 137CA | 60.54 |
| 98OD1 | 287ZN | 138CB | 55.97 |
| 98OD1 | 287ZN | 99O | 46.43 |
| 98OD1 | 287ZN | 136CA | 83.96 |
| 98OD1 | 287ZN | 138CA | 44.98 |
| 98OD1 | 287ZN | 99N | 19.36 |
| 98OD1 | 287ZN | 264CB | 94.81 |
| 98OD1 | 287ZN | 100CG2 | 81.89 |
| 98OD1 | 287ZN | 98CA | 15.47 |
| 98OD1 | 287ZN | 136O | 89.20 |
| 98CG | 287ZN | 98CB | 3.38 |
| 98CG | 287ZN | 137N | 90.56 |
| 98CG | 287ZN | 138N | 61.38 |
| 98CG | 287ZN | 137C | 64.66 |
| 98CG | 287ZN | 137CA | 83.55 |
| 98CG | 287ZN | 138CB | 46.58 |
| 98CG | 287ZN | 99O | 73.04 |
| 98CG | 287ZN | 138CA | 47.31 |
| 98CG | 287ZN | 99N | 40.72 |
| 98CG | 287ZN | 264CB | 95.54 |
| 98CG | 287ZN | 100CG2 | 98.06 |
| 98CG | 287ZN | 98CA | 13.98 |
| 136CG | 287ZN | 264CD | 76.76 |
| 136CG | 287ZN | 264OE2 | 62.66 |
| 136CG | 287ZN | 136OD1 | 22.84 |
| 136CG | 287ZN | 137N | 58.63 |
| 136CG | 287ZN | 136CB | 9.82 |
| 136CG | 287ZN | 264CG | 80.95 |
| 136CG | 287ZN | 136C | 42.30 |
| 136CG | 287ZN | 137C | 92.06 |
| 136CG | 287ZN | 137CA | 73.70 |
| 136CG | 287ZN | 232CD | 95.72 |
| 136CG | 287ZN | 232OE2 | 80.72 |
| 136CG | 287ZN | 99O | 66.37 |
| 136CG | 287ZN | 136CA | 28.84 |
| 136CG | 287ZN | 99N | 98.91 |
| 136CG | 287ZN | 264CB | 98.16 |
| 136CG | 287ZN | 100CG2 | 51.97 |
| 136CG | 287ZN | 136O | 43.17 |
| 264CD | 287ZN | 264OE2 | 23.07 |
| 264CD | 287ZN | 136OD1 | 76.24 |
| 264CD | 287ZN | 232OE1 | 63.17 |
| 264CD | 287ZN | 137N | 70.41 |
| 264CD | 287ZN | 138N | 63.68 |
| 264CD | 287ZN | 136CB | 77.16 |
| 264CD | 287ZN | 264CG | 9.03 |
| 264CD | 287ZN | 136C | 64.48 |
| 264CD | 287ZN | 137C | 70.17 |
| 264CD | 287ZN | 137CA | 60.22 |
| 264CD | 287ZN | 138CB | 74.08 |
| 264CD | 287ZN | 232CD | 51.78 |
| 264CD | 287ZN | 232OE2 | 51.48 |
| 264CD | 287ZN | 136CA | 78.38 |
| 264CD | 287ZN | 138CA | 74.06 |
| 264CD | 287ZN | 264CB | 24.80 |
| 264CD | 287ZN | 136O | 51.35 |
| 264OE2 | 287ZN | 136OD1 | 55.85 |
| 264OE2 | 287ZN | 232OE1 | 57.71 |
| 264OE2 | 287ZN | 137N | 79.65 |
| 264OE2 | 287ZN | 138N | 85.96 |
| 264OE2 | 287ZN | 136CB | 66.49 |
| 264OE2 | 287ZN | 264CG | 31.91 |
| 264OE2 | 287ZN | 136C | 67.60 |
| 264OE2 | 287ZN | 137C | 89.73 |
| 264OE2 | 287ZN | 137CA | 75.65 |
| 264OE2 | 287ZN | 138CB | 96.22 |
| 264OE2 | 287ZN | 232CD | 43.32 |
| 264OE2 | 287ZN | 232OE2 | 35.86 |
| 264OE2 | 287ZN | 136CA | 74.96 |
| 264OE2 | 287ZN | 138CA | 97.11 |
| 264OE2 | 287ZN | 264CB | 47.22 |
| 264OE2 | 287ZN | 136O | 54.51 |
| 136OD1 | 287ZN | 232OE1 | 89.01 |
| 136OD1 | 287ZN | 137N | 80.41 |
| 136OD1 | 287ZN | 136CB | 32.66 |
| 136OD1 | 287ZN | 264CG | 83.25 |
| 136OD1 | 287ZN | 136C | 63.25 |
| 136OD1 | 287ZN | 137CA | 93.36 |
| 136OD1 | 287ZN | 232CD | 77.59 |
| 136OD1 | 287ZN | 232OE2 | 62.09 |
| 136OD1 | 287ZN | 99O | 86.50 |
| 136OD1 | 287ZN | 136CA | 51.64 |
| 136OD1 | 287ZN | 100CG2 | 59.94 |
| 136OD1 | 287ZN | 136O | 60.50 |
| 232OE1 | 287ZN | 264CG | 68.41 |
| 232OE1 | 287ZN | 138CB | 85.49 |
| 232OE1 | 287ZN | 232CD | 14.69 |
| 232OE1 | 287ZN | 232OE2 | 28.30 |
| 232OE1 | 287ZN | 264CB | 66.44 |
| 98CB | 287ZN | 137N | 93.28 |
| 98CB | 287ZN | 138N | 64.75 |
| 98CB | 287ZN | 137C | 67.95 |
| 98CB | 287ZN | 137CA | 86.76 |
| 98CB | 287ZN | 138CB | 49.16 |
| 98CB | 287ZN | 99O | 73.82 |
| 98CB | 287ZN | 138CA | 50.57 |
| 98CB | 287ZN | 99N | 41.25 |
| 98CB | 287ZN | 264CB | 98.31 |
| 98CB | 287ZN | 100CG2 | 96.77 |
| 98CB | 287ZN | 98CA | 16.02 |
| 137N | 287ZN | 138N | 49.38 |
| 137N | 287ZN | 136CB | 49.25 |
| 137N | 287ZN | 264CG | 65.52 |
| 137N | 287ZN | 136C | 17.56 |
| 137N | 287ZN | 137C | 33.89 |
| 137N | 287ZN | 137CA | 18.89 |
| 137N | 287ZN | 138CB | 81.50 |
| 137N | 287ZN | 99O | 43.79 |
| 137N | 287ZN | 136CA | 31.37 |
| 137N | 287ZN | 138CA | 63.88 |
| 137N | 287ZN | 99N | 63.76 |
| 137N | 287ZN | 264CB | 71.43 |
| 137N | 287ZN | 100CG2 | 75.86 |
| 137N | 287ZN | 98CA | 77.57 |
| 137N | 287ZN | 136O | 26.64 |
| 138N | 287ZN | 136CB | 96.89 |
| 138N | 287ZN | 264CG | 54.65 |
| 138N | 287ZN | 136C | 63.33 |
| 138N | 287ZN | 137C | 17.28 |
| 138N | 287ZN | 137CA | 31.79 |
| 138N | 287ZN | 138CB | 32.35 |
| 138N | 287ZN | 99O | 75.48 |
| 138N | 287ZN | 136CA | 80.51 |
| 138N | 287ZN | 138CA | 16.59 |
| 138N | 287ZN | 99N | 67.54 |
| 138N | 287ZN | 264CB | 44.56 |
| 138N | 287ZN | 98CA | 55.90 |
| 138N | 287ZN | 136O | 62.74 |
| 136CB | 287ZN | 264CG | 79.89 |
| 136CB | 287ZN | 136C | 33.58 |
| 136CB | 287ZN | 137C | 82.96 |
| 136CB | 287ZN | 137CA | 65.16 |
| 136CB | 287ZN | 232OE2 | 88.66 |
| 136CB | 287ZN | 99O | 58.24 |
| 136CB | 287ZN | 136CA | 19.02 |
| 136CB | 287ZN | 99N | 90.88 |
| 136CB | 287ZN | 264CB | 96.11 |
| 136CB | 287ZN | 100CG2 | 50.97 |
| 136CB | 287ZN | 136O | 36.70 |
| 264CG | 287ZN | 136C | 62.27 |
| 264CG | 287ZN | 137C | 61.70 |
| 264CG | 287ZN | 137CA | 53.30 |
| 264CG | 287ZN | 138CB | 66.38 |
| 264CG | 287ZN | 232CD | 58.39 |
| 264CG | 287ZN | 232OE2 | 59.72 |
| 264CG | 287ZN | 136CA | 78.10 |
| 264CG | 287ZN | 138CA | 65.22 |
| 264CG | 287ZN | 264CB | 17.77 |
| 264CG | 287ZN | 136O | 49.92 |
| 136C | 287ZN | 137C | 49.81 |
| 136C | 287ZN | 137CA | 31.58 |
| 136C | 287ZN | 138CB | 95.59 |
| 136C | 287ZN | 99O | 51.08 |
| 136C | 287ZN | 136CA | 18.96 |
| 136C | 287ZN | 138CA | 79.10 |
| 136C | 287ZN | 99N | 77.51 |
| 136C | 287ZN | 264CB | 73.28 |
| 136C | 287ZN | 100CG2 | 71.42 |
| 136C | 287ZN | 98CA | 94.62 |
| 136C | 287ZN | 136O | 13.48 |
| 137C | 287ZN | 137CA | 19.50 |
| 137C | 287ZN | 138CB | 47.95 |
| 137C | 287ZN | 99O | 59.16 |
| 137C | 287ZN | 136CA | 65.23 |
| 137C | 287ZN | 138CA | 30.03 |
| 137C | 287ZN | 99N | 57.65 |
| 137C | 287ZN | 264CB | 56.53 |
| 137C | 287ZN | 98CA | 55.11 |
| 137C | 287ZN | 136O | 52.73 |
| 137CA | 287ZN | 138CB | 64.12 |
| 137CA | 287ZN | 99O | 57.85 |
| 137CA | 287ZN | 136CA | 48.96 |
| 137CA | 287ZN | 138CA | 47.59 |
| 137CA | 287ZN | 99N | 68.34 |
| 137CA | 287ZN | 264CB | 55.04 |
| 137CA | 287ZN | 100CG2 | 94.22 |
| 137CA | 287ZN | 98CA | 72.83 |
| 137CA | 287ZN | 136O | 33.23 |
| 138CB | 287ZN | 232CD | 90.81 |
| 138CB | 287ZN | 99O | 97.50 |
| 138CB | 287ZN | 138CA | 18.33 |
| 138CB | 287ZN | 99N | 75.23 |
| 138CB | 287ZN | 264CB | 49.30 |
| 138CB | 287ZN | 98CA | 50.29 |
| 138CB | 287ZN | 136O | 93.47 |
| 232CD | 287ZN | 232OE2 | 15.49 |
| 232CD | 287ZN | 264CB | 60.67 |
| 232CD | 287ZN | 136O | 97.68 |
| 232OE2 | 287ZN | 264CB | 67.06 |
| 232OE2 | 287ZN | 136O | 89.33 |
| 99O | 287ZN | 136CA | 44.16 |
| 99O | 287ZN | 138CA | 80.94 |
| 99O | 287ZN | 99N | 32.65 |
| 99O | 287ZN | 100CG2 | 42.76 |
| 99O | 287ZN | 98CA | 59.92 |
| 99O | 287ZN | 136O | 64.54 |
| 136CA | 287ZN | 138CA | 95.25 |
| 136CA | 287ZN | 99N | 75.75 |
| 136CA | 287ZN | 264CB | 91.12 |
| 136CA | 287ZN | 100CG2 | 53.46 |
| 136CA | 287ZN | 98CA | 99.35 |
| 136CA | 287ZN | 136O | 28.47 |
| 138CA | 287ZN | 99N | 63.97 |
| 138CA | 287ZN | 264CB | 51.18 |
| 138CA | 287ZN | 98CA | 45.03 |
| 138CA | 287ZN | 136O | 79.30 |
| 99N | 287ZN | 100CG2 | 62.83 |
| 99N | 287ZN | 98CA | 28.56 |
| 99N | 287ZN | 136O | 89.76 |
| 264CB | 287ZN | 98CA | 96.08 |
| 264CB | 287ZN | 136O | 62.75 |
| 100CG2 | 287ZN | 98CA | 89.80 |
| 100CG2 | 287ZN | 136O | 81.88 |
| 264OE2 | 288ZN | 136OD1 | 92.52 |
| 264OE2 | 288ZN | 232OE2 | 81.54 |
| 264OE2 | 288ZN | 232CD | 75.02 |
| 264OE2 | 288ZN | 136CG | 75.50 |
| 264OE2 | 288ZN | 264CD | 15.25 |
| 264OE2 | 288ZN | 232OE1 | 80.90 |
| 264OE2 | 288ZN | 136OD2 | 67.40 |
| 264OE2 | 288ZN | 264OE1 | 35.47 |
| 264OE2 | 288ZN | 230OG1 | 72.15 |
| 264OE2 | 288ZN | 230CG2 | 63.00 |
| 264OE2 | 288ZN | 230CB | 56.16 |
| 264OE2 | 288ZN | 136CB | 74.97 |
| 264OE2 | 288ZN | 232CG | 66.62 |
| 264OE2 | 288ZN | 264CG | 6.40 |
| 264OE2 | 288ZN | 232CB | 50.24 |
| 264OE2 | 288ZN | 98OD2 | 74.73 |
| 136OD1 | 288ZN | 199NE2 | 89.61 |
| 136OD1 | 288ZN | 136CG | 19.54 |
| 136OD1 | 288ZN | 199CE1 | 70.81 |
| 136OD1 | 288ZN | 264CD | 92.63 |
| 136OD1 | 288ZN | 136OD2 | 39.10 |
| 136OD1 | 288ZN | 264OE1 | 87.68 |
| 136OD1 | 288ZN | 230CG2 | 76.96 |
| 136OD1 | 288ZN | 230CB | 98.46 |
| 136OD1 | 288ZN | 199ND1 | 82.45 |
| 136OD1 | 288ZN | 136CB | 18.07 |
| 136OD1 | 288ZN | 264CG | 97.29 |
| 136OD1 | 288ZN | 98OD2 | 87.62 |
| 199NE2 | 288ZN | 232OE2 | 87.65 |
| 199NE2 | 288ZN | 199CE1 | 21.48 |
| 199NE2 | 288ZN | 199CD2 | 21.37 |
| 199NE2 | 288ZN | 230OG1 | 66.77 |
| 199NE2 | 288ZN | 230CG2 | 74.80 |
| 199NE2 | 288ZN | 230CB | 80.02 |
| 199NE2 | 288ZN | 199ND1 | 13.14 |
| 199NE2 | 288ZN | 199CG | 12.54 |
| 199NE2 | 288ZN | 136CB | 99.19 |
| 232OE2 | 288ZN | 232CD | 20.97 |
| 232OE2 | 288ZN | 199CD2 | 67.12 |
| 232OE2 | 288ZN | 264CD | 84.34 |
| 232OE2 | 288ZN | 232OE1 | 42.10 |
| 232OE2 | 288ZN | 264OE1 | 93.22 |
| 232OE2 | 288ZN | 230OG1 | 55.14 |
| 232OE2 | 288ZN | 230CG2 | 91.22 |
| 232OE2 | 288ZN | 230CB | 69.74 |
| 232OE2 | 288ZN | 199ND1 | 92.54 |
| 232OE2 | 288ZN | 199CG | 75.60 |
| 232OE2 | 288ZN | 232CG | 18.73 |
| 232OE2 | 288ZN | 264CG | 77.64 |
| 232OE2 | 288ZN | 232CB | 35.89 |
| 232CD | 288ZN | 199CD2 | 84.98 |
| 232CD | 288ZN | 264CD | 72.72 |
| 232CD | 288ZN | 232OE1 | 22.53 |
| 232CD | 288ZN | 264OE1 | 76.40 |
| 232CD | 288ZN | 230OG1 | 72.79 |
| 232CD | 288ZN | 230CB | 83.02 |
| 232CD | 288ZN | 199CG | 94.89 |
| 232CD | 288ZN | 232CG | 11.65 |
| 232CD | 288ZN | 264CG | 69.60 |
| 232CD | 288ZN | 232CB | 24.79 |
| 232CD | 288ZN | 98OD2 | 79.59 |
| 136CG | 288ZN | 199CE1 | 88.59 |
| 136CG | 288ZN | 264CD | 73.67 |
| 136CG | 288ZN | 136OD2 | 21.49 |
| 136CG | 288ZN | 264OE1 | 68.25 |
| 136CG | 288ZN | 230CG2 | 77.94 |
| 136CG | 288ZN | 230CB | 96.39 |
| 136CG | 288ZN | 136CB | 13.55 |
| 136CG | 288ZN | 264CG | 79.55 |
| 136CG | 288ZN | 98OD2 | 74.13 |
| 199CE1 | 288ZN | 199CD2 | 41.86 |
| 199CE1 | 288ZN | 230OG1 | 67.52 |
| 199CE1 | 288ZN | 230CG2 | 61.35 |
| 199CE1 | 288ZN | 230CB | 73.57 |
| 199CE1 | 288ZN | 199ND1 | 11.65 |
| 199CE1 | 288ZN | 199CG | 30.28 |
| 199CE1 | 288ZN | 136CB | 78.25 |
| 199CD2 | 288ZN | 232OE1 | 96.67 |
| 199CD2 | 288ZN | 230OG1 | 60.64 |
| 199CD2 | 288ZN | 230CG2 | 82.07 |
| 199CD2 | 288ZN | 230CB | 79.08 |
| 199CD2 | 288ZN | 199ND1 | 31.07 |
| 199CD2 | 288ZN | 199CG | 12.47 |
| 199CD2 | 288ZN | 232CG | 85.85 |
| 264CD | 288ZN | 232OE1 | 72.89 |
| 264CD | 288ZN | 136OD2 | 60.52 |
| 264CD | 288ZN | 264OE1 | 20.48 |
| 264CD | 288ZN | 230OG1 | 86.11 |
| 264CD | 288ZN | 230CG2 | 77.76 |
| 264CD | 288ZN | 230CB | 71.24 |
| 264CD | 288ZN | 136CB | 76.83 |
| 264CD | 288ZN | 232CG | 66.90 |
| 264CD | 288ZN | 264CG | 11.89 |
| 264CD | 288ZN | 232CB | 49.27 |
| 264CD | 288ZN | 98OD2 | 59.51 |
| 232OE1 | 288ZN | 264OE1 | 68.35 |
| 232OE1 | 288ZN | 230OG1 | 95.30 |
| 232OE1 | 288ZN | 232CG | 32.73 |
| 232OE1 | 288ZN | 264CG | 74.59 |
| 232OE1 | 288ZN | 232CB | 36.87 |
| 232OE1 | 288ZN | 98OD2 | 59.59 |
| 136OD2 | 288ZN | 264OE1 | 49.85 |
| 136OD2 | 288ZN | 230CG2 | 91.95 |
| 136OD2 | 288ZN | 136CB | 33.93 |
| 136OD2 | 288ZN | 264CG | 69.46 |
| 136OD2 | 288ZN | 98OD2 | 53.31 |
| 264OE1 | 288ZN | 230CG2 | 95.97 |
| 264OE1 | 288ZN | 230CB | 91.63 |
| 264OE1 | 288ZN | 136CB | 75.94 |
| 264OE1 | 288ZN | 232CG | 74.50 |
| 264OE1 | 288ZN | 264CG | 32.27 |
| 264OE1 | 288ZN | 232CB | 57.66 |
| 264OE1 | 288ZN | 98OD2 | 39.41 |
| 230OG1 | 288ZN | 230CG2 | 37.69 |
| 230OG1 | 288ZN | 230CB | 21.01 |
| 230OG1 | 288ZN | 199ND1 | 60.54 |
| 230OG1 | 288ZN | 199CG | 57.00 |
| 230OG1 | 288ZN | 232CG | 63.16 |
| 230OG1 | 288ZN | 264CG | 74.22 |
| 230OG1 | 288ZN | 232CB | 67.83 |
| 230CG2 | 288ZN | 230CB | 22.15 |
| 230CG2 | 288ZN | 199ND1 | 62.50 |
| 230CG2 | 288ZN | 199CG | 72.14 |
| 230CG2 | 288ZN | 136CB | 65.36 |
| 230CG2 | 288ZN | 232CG | 93.81 |
| 230CG2 | 288ZN | 264CG | 68.45 |
| 230CG2 | 288ZN | 232CB | 90.19 |
| 230CB | 288ZN | 199ND1 | 70.40 |
| 230CB | 288ZN | 199CG | 72.83 |
| 230CB | 288ZN | 136CB | 85.06 |
| 230CB | 288ZN | 232CG | 71.75 |
| 230CB | 288ZN | 264CG | 59.82 |
| 230CB | 288ZN | 232CB | 69.74 |
| 199ND1 | 288ZN | 199CG | 18.99 |
| 199ND1 | 288ZN | 136CB | 89.26 |
| 199CG | 288ZN | 232CG | 94.03 |
| 136CB | 288ZN | 264CG | 80.09 |
| 136CB | 288ZN | 98OD2 | 87.17 |
| 232CG | 288ZN | 264CG | 61.80 |
| 232CG | 288ZN | 232CB | 17.67 |
| 232CG | 288ZN | 98OD2 | 85.44 |
| 264CG | 288ZN | 232CB | 44.93 |
| 264CG | 288ZN | 98OD2 | 70.81 |
| 232CB | 288ZN | 98OD2 | 75.59 |

The above description fully discloses the invention including certain embodiments thereof. Modifications and improvements of the embodiments specifically disclosed herein are within the scope of the following claims. Without further elaboration it is believed that one skilled in the art can, given the preceding description, utilize the present invention to its fullest extent. Therefore any examples are to be construed as merely illustrative and not a limitation on the scope of the present invention in any way. The embodiments of the invention in which an exclusive property or privilege is claimed are defined as follows.

1. A composition comprising a bacterial MetAP in crystalline form.

2. The composition according to claim 1 wherein said MetAP is a Staphylococcus aureus MetAP.

3. The composition according to claim 2, wherein the crystalline form is defined by three dimensional protein coordinates of Table I in an essentially pure form or a homolog thereof.

4. The composition according to claim 2, wherein said crystalline form comprises cubic crystals with a space group I23.

5. The composition according to claim 4, wherein said cubic crystals comprise a lattice constant of a=121.36 Ångstroms (Å).

6. The composition according to claim 2, wherein said crystalline form comprises monoclinic crystals with a space group P2₁.

7. The composition according to claim 6, wherein said monoclinic crystals comprise lattice constants of a=41.19 Å, b=76.78 Å, and c=41.71 Å, β=104.165°.

8. A composition comprising a Staphylococcus aureus MetAP in complex with a MetAP inhibitor.

9. The composition according to claim 8, wherein said MetAP inhibitor is a 1,2,3 triazole.

10. The composition according to claim 8 wherein the MetAP inhibitor is selected from the group consisting of 5-(3-Iodo-phenyl)-1-H-[1,2,3]triazole and 5-benzofuran-2-yl-1-H-[1,2,3]triazole.

11-18. (canceled)

19. A process of identifying a bacterial MetAP inhibitor capable of binding to and inhibiting the enzymatic activity of a bacterial MetAP said process comprising:
(a) introducing into a suitable computer program information defining an active site conformation of a MetAP molecule comprising a conformation defined by MetAP crystal coordinates and listed in Tables I to X wherein said program displays a three-dimensional structure;
(b) creating a three dimensional structure of a test compound in said computer program;
(c) displaying and superimposing a model of said test compound on a model of said active site;
(d) incorporating said test compound in a biological methionine aminopeptidase activity assay for a methionine aminopeptidase characterized by said active site; and
(e) determining whether said test compound inhibits enzymatic activity in said assay.

20. (canceled)

21. A method of modifying a test bacterial MetAP polypeptide comprising:
providing a test bacterial MetAP polypeptide sequence having a characteristic that is targeted for modification;
aligning the test bacterial MetAP polypeptide sequence with a reference bacterial MetAP polypeptide sequence for which an X-ray structure is available, wherein a reference bacterial MetAP polypeptide sequence has a characteristic that is desired for the test bacterial MetAP polypeptide;
building a three-dimensional model for the test bacterial MetAP polypeptide using the three-dimensional coordinates of the X-ray structure(s) of a reference bacterial MetAP polypeptide and its sequence alignment with the test bacterial MetAP polypeptide sequence;
examining the three-dimensional model of the test bacterial MetAP polypeptide for a difference in an amino acid residue as compared to a reference bacterial MetAP polypeptide, wherein the residues are associated with the desired characteristic; and
mutating an amino acid residue in the test bacterial MetAP polypeptide sequence located at a difference identified in step (d) to a residue associated with the desired characteristic, whereby the test bacterial MetAP polypeptide is modified.

22-23. (canceled)
